(19) United States
(12) Patent Application Publication
(10) Pub. No.: US 2026/0273095 A1
(43) Pub. Date: Sep. 17, 2026

(54) DELIVERY OF GENE EDITING SYSTEMS AND METHODS OF USE THEREOF

(71) Applicant: ReNAgade Therapeutics Management Inc., Watertown, MA (US)

(72) Inventors: Muthusamy Jayaraman, Walpole, MA (US); Ganapathy Subramanian Sankaran, Ashland, MA (US)

(21) Appl. No.: 19/163,776

(22) PCT Filed: Mar. 14, 2024
(86) PCT No.: PCT/US2024/020008

§ 371 Date: Sep. 9, 2025

Related U.S. Application Data
(60) Provisional application No. 63/452,316, filed on Mar. 15, 2023.

(51) Int. Cl. A61K 48/00; A61K 9/51; A61K 31/7105; A61K 47/18; A61K 47/24; C12N 9/22; C12N 15/113

(52) U.S. Cl. CPC A61K 48/0025; A61K 9/5123; A61K 31/7105; A61K 47/18; A61K 47/24; A61K 48/005; C12N 9/222; C12N 15/113; C12N 2310/20

(57) ABSTRACT

The present disclosure describes improved LNP-based nucleobase editing systems and therapeutics for use in treating a disease. In particular, the disclosure describes improved LNPs, including novel and improved ionic lipids for making LNPs, that enhance the targeted delivery of LNP-based nucleobase editing systems and therapeutics based on linear and/or circular mRNAs. The improved LNPs protect linear and/or circular mRNA payloads from degradation and clearance while achieving targeted systemic or local delivery for use as enhanced nucleobase editing systems and/or therapeutic agents.

### TECHNICAL FIELD

The present disclosure generally relates to the field of nucleic acid lipid nanoparticle (LNP) compositions and use thereof in the delivery of nucleobase editing systems. The disclosure further relates to compositions comprising LNPs formulated with coding RNAs, including linear and/or circular mRNAs, for the delivery of encoded nucleobase editing systems.

### SEQUENCE LISTING

The instant application contains a Sequence Listing which has been submitted electronically in xml format and is hereby incorporated by reference in its entirety. The xml copy, created on Feb. 15, 2024, is named RNG012-WO1.xml and is 44,129 bytes in size.

### BACKGROUND

There are many challenges associated with the delivery of nucleobase editing systems to affect a desired edit, modification, or alteration of a target polynucleotide sequence in a biological system. Nucleic acid-based therapeutics have enormous potential but there remains a need for more effective delivery of nucleic acids to appropriate sites within a cell or organism in order to realize this potential.

Genome editing tools encompass a diverse set of technologies that can make many types of genomic alterations in various contexts. These technologies have evolved over the last couple of decades to provide a range of user-programmable editing tools that include ZFN (zinc finger nuclease) editing systems, meganuclease editing systems, and TALENS (transcription activator-like effector nucleases). The past decade has seen an explosive growth in a new generation of genome editing systems based on components from bacterial immune pathways, including CRISPR (clustered regularly interspaced short palindromic repeats) and the associated CRISPR-associated proteins (e.g., CRISPR-Cas9) (Jinek et al., “A programmable dual-RNA-guided DNA endonuclease in adaptive bacterial immunity,” Science, Vol. 337 (6096), pp. 816-821), meganuclease editors (Boissel et al., “megaTALs: a rare-cleaving nuclease architecture for therapeutic genome engineering,” Nucleic Acids Research 42: pp. 2591-2601) and bacterial retron systems (Schubert et al., “High-throughput functional variant screens via in vivo production of single-stranded DNA,” PNAS, Apr. 27, 2021, Vol. 118(18), pp. 1-10). In particular, CRISPR-Cas9 has been derivatized in numerous ways to expand upon its guide RNA-based programmable double-strand cutting activity to form systems ranging from finding alternative CRISPR Cas nuclease enzymes having different PAM requirements and cutting properties (e.g., Cas12a, Cas12f, Cas13a, and Cas13b) to base editing (Komor et al., “Programmable editing of a target base in genomic DNA without double-stranded DNA cleavage,” Nature, May 19, 2016, 533 (7603); pp. 420-424 [cytosine base editors or CBEs] and Gaudelli et al., “Programmable base editing of A-T to G-C in genomic DNA without DNA cleavage,” Nature, Vol. 551, pp. 464-471 [adenine base editors or ABEs]) to prime editing (Anzalone et al., “Search-and-replace genome editing without double-strand breaks or donor DNA,” Nature, December 2019, 576 (7789): pp. 149-157) to twin prime editing (Anzalone et al., “Programmable deletion, replacement, integration and inversion of large DNA sequences with twin prime editing,” Nature Biotechnology, Dec. 9, 2021, vol. 40, pp. 731-740) to epigenetic editing (Kungulovski and Jeltsch, “Epigenome Editing: State of the Art, Concepts, and Perspective,” Trends in Genetics, Vol. 32, 206, pp. 101-113) to CRISPR-directed integrase editing (Yarnell et al., “Drag-and-drop genome insertion of large sequences without double-stranded DNA cleavage using CRISPR-directed integrases,” Nature Biotechnology, Nov. 24, 2022, (“PASTE”)).

While the expansion of genome editing tools has exploded, the development of safe and effective gene editing tool delivery systems has lagged behind. There remain numerous challenges associated with the delivery of gene editing tools—including, but not limited to, CRISPR-Cas9 and alternative Cas nuclease editors, retron editors, base editors, prime editors, twin prime editors, epigenetic editors, and integrase editors—to achieve safe and effective therapeutic application of such tools in cells and patients for treating disease and/or otherwise modifying the nucleotide sequence of a target nucleic acid molecule (e.g., a gene or genome). That said, the use of lipid nanoparticles (LNPs) has emerged as a leading delivery option for the safe, effective, and targeted delivery of gene editing tools to target tissues and cells. However, there remains a need for improved LNPs, including better performing ionic lipids, that will enhance the targeted delivery of LNP-based gene editing tools. Preferably, such improved LNPs would protect payloads from degradation and clearance while achieving targeted delivery, be suitable for systemic or local delivery, and provide delivery of a wide variety of gene editing tools, such as those mentioned above. In addition, such improved LNP-based therapeutics should exhibit low toxicity and provide an adequate therapeutic index, such that patient treatment at an effective dose of the LNP minimizes risk to the patient while maximizing therapeutic benefit. The present invention provides these and related advantages.

### SUMMARY

Described herein are compositions, methods, processes, and kits for the selection, design, preparation, manufacture, formulation, and/or use of LNP-based nucleobase editing systems and therapeutics comprising the same. In particular, described herein are compositions, methods, processes, and kits comprising RNA based nucleobase editing systems as part of an LNP formulation.

### DETAILED DESCRIPTION

### I. Introduction

Described herein are LNP compositions comprising gene editing systems for use in treating disease and/or otherwise modifying the sequence and/or expression of target nucleotide sequences. The disclosure provides LNPs capable of delivering a gene editing system to a target organ, tissue, and/or cell. The gene editing systems may be delivered to cells under in vitro or ex vivo conditions and to organs, tissues, or cells under in vivo conditions (e.g., administered to a subject in an effective amount).

The disclosure also provides in various aspects therapeutic or pharmaceutical compositions comprising LNPs comprising gene editing systems or one or more components thereof. The gene editing systems may comprise DNA components, RNA components, protein components, nucleoprotein components, polysaccharide components, or combinations thereof. In other aspects, the disclosure provides nucleic acid molecules that encode various componentry of the deliverable gene editing systems contemplated herein. In addition, other aspects of the disclosure provide nucleic acid molecules as components of the herein contemplated gene editing systems, such as, but not limited to plasmids or vectors encoding one or more components of a gene editing system, RNAs encoding one or more components of a gene editing system (e.g., mRNAs coding for a nuclease domain of a gene editing system), and non-coding RNAs (e.g., guide RNAs capable of complexing with and targeting a nucleic acid-programmable DNA binding domain to a specific target nucleotide sequence or a retron ncRNAs). The disclosure in other aspects provides for the various protein components of the various gene editing systems contemplated herein, including, but not limited to, user-programmable DNA binding proteins and various effector proteins, such as nucleases, polymerases, reverse transcriptases, recombinases, integrases, endonucleases, exonucleases, transposases, and deaminases. The disclosure also describes nucleoprotein components of the gene editing systems contemplated herein, such as, but not limited to a nuclease-guide RNA complexes. The disclosure also provides methods of modifying the sequence and/or expression level of a target nucleic acid molecule through the delivery and/or administration of an LNP described herein that comprises a gene editing system or components thereof. Still further, the disclosure provides methods of treating a disease by administering a therapeutically effective amount of an LNP-based gene editing system that results in the modification in the sequence and/or expression level of a target nucleic acid molecule (e.g., a disease-associated gene).

The gene editing systems deliverable by the herein disclosed LNPs can be any gene editing system. The gene editing systems contemplated herein can include (A) nucleobase gene editing systems which result in one or more the modifications to the sequence of target nucleic acid molecule (e.g., a gene or gene regulatory sequence), (B) an epigenetic editing system which results in one or more modifications to the epigenome to bring about an effect on gene expression without altering the sequence of a nucleic acid molecule, and (C) gene editing systems that combine the features of nucleobase editing systems and epigenetic editing systems.

Nucleobase editing systems include a wide array of configurations with various combinations of protein functionalities and/or nucleic acid molecule components, all of which are contemplated herein. In general, nucleobase editing systems comprise at least a (i) DNA binding domain that is user-programmable to target a specific sequence in a nucleic acid molecule and optionally (ii) one or more effector domains that facilitate the modification of the sequence of the nucleic acid molecule. User-programmability may comprise amino acid sequence-programmable DNA binding domains (e.g., TALENS, zinc finger-binding domains, meganucleases (or homing endonucleases)) or nucleic acid sequence-programmable DNA binding domains (e.g., CRISPR Cas9, CRISPR Cas12a, CRISPR Cas12f, CRISPR Cas13a, CRISPR Cas13b, or TnpB). Similarly, epigenetic editing systems comprise at least a (i) DNA binding domain that targets a specific sequence in a nucleic acid molecule and (ii) one or more effector domains that facilitates the modification of one or more epigenomic features of the nucleic acid molecule.

Gene editing systems may comprise one or more effector domains that provide various functionalities that facilitate changes in nucleotide sequence and/or gene expression, such as, but not limited to, single-strand DNA binding proteins, nucleases, endonucleases, exonucleases, deaminases (e.g., cytidine deaminases or adenosine deaminases), polymerases (e.g., reverse transcriptases), integrases, recombinases, etc., and fusion proteins comprising one or more functional domains linked together.

In addition, gene editing systems that utilize a nucleic acid sequence-programmable DNA binding domain may also comprise one or more non-coding nucleic acids, such as, one or more guide RNAs which complex with the nucleic acid programmable DNA binding protein and target the complex to a specific nucleotide sequence. In the case of prime editing, the guide RNA may be a prime editing guide RNA (“pegRNA”) which comprises a specialized extended region of RNA the provides a template sequence of a reverse transcriptase. Other specialized guide RNAs may be included depending upon the requirements and/or nature of the gene editing system and the cognate nucleic acid programmable proteins. For example, TnpB enzymes require a specialized guide RNA referred to as reRNA. Also, guide RNAs have different characteristics (e.g., PAM preferences, the spacer length, and the scaffold portion that binds to the nuclease protein) depending upon the programmable nuclease requirements.

The gene editing systems contemplated here may introduce a wide variety of changes, including (A) a change in the sequence of the target nucleic acid molecule, such as, but not limited to, (i) a nucleobase substitution (e.g., a purine to a pyrimidine), (ii) a deletion of one or more nucleobases, (iii) an insertion of one or more nucleobases, (iv) a combination of a deletion and insertion of one or more nucleobases, (v) an inversion of a nucleobase sequence, a (vi) translocation of a nucleobase sequence, and (vii) a combination or two or more such modifications, and (B) one or more modifications to the epigenome to bring about an effect on gene expression without altering the sequence of a nucleic acid molecule wherein said epigenetic change results in altered gene expression through altered chromatin structure or accessibility.

The LNP compositions and/or gene editing systems described herein may include a variety of coding RNA molecules that code for the various components of gene editors. In various aspects, the coding RNA may be linear mRNA. In other embodiments, the coding RNA may be circular mRNA. In various aspects, the improved LNPs protect linear and/or circular mRNA cargos from degradation and clearance while achieving targeted systemic or local delivery for use as enhanced gene editing platforms and/or therapeutic agents.

In various other aspects, the LNP compositions and/or gene editing systems described herein may also include a repair template, e.g., a repair.

Accordingly, the instant specification describes compositions, methods, processes, kits and devices for the selection, design, preparation, manufacture, formulation, and/or use of LNP-based gene editing systems as therapeutic compositions. Further described herein are compositions, methods, processes, kits and devices for the selection, design, preparation, manufacture, formulation, and/or use of LNP-based gene editing therapeutics for the prophylactic and/or therapeutic treatment of one or more diseases or a symptom thereof. The components capable of being encapsulated by or otherwise incorporated by the LNPs described herein may be referred to as LNP “payloads” and may include all of the biological materials described above, including DNA molecules, RNA molecules (coding and/or non-coding), proteins, and nucleoproteins (e.g., Cas/guide RNA complexes)

### II. LNP Delivery Systems

The RNA payloads (e.g., linear and circular mRNAs) described herein may be encapsulated and delivered by lipid nanoparticles (LNPs) and compositions and/or formulations comprising RNA-encapsulated LNPs.

Below describes LNPs that may be used as the RNA payload delivery vehicles contemplated herein, as well as the various ionizable lipids, structural lipids, PEGylated lipids, and phospholipids that may be used to make the herein LNPs for delivery RNA payloads to cells. In addition, below describes additional LNP components that are contemplated, such as targeting moieties and other lipid components.

### A. Lipid Nanoparticle Compositions

In one aspect, the present disclosure further provides delivery systems for delivery of a therapeutic payload (e.g., the RNA payloads described herein which may encode a polypeptide of interest, e.g., a nucleobase editing system or a therapeutic protein) disclosed herein. In some embodiments, a delivery system suitable for delivery of the therapeutic payload disclosed herein comprises a lipid nanoparticle (LNP) formulation.

In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid. In some embodiments, an LNP further comprises a 5th lipid, besides any of the aforementioned lipid components. In some embodiments, the LNP encapsulates one or more elements of the active agent of the present disclosure. In some embodiments, an LNP further comprises a targeting moiety covalently or non-covalently bound to the outer surface of the LNP. In some embodiments, the targeting moiety is a targeting moiety that binds to, or otherwise facilitates uptake by, cells of a particular organ system.

In some embodiments, an LNP has a diameter of at least about 20 nm, 30 nm, 40 nm, 50 nm, 60 nm, 70 nm, 80 nm, or 90 nm. In some embodiments, an LNP has a diameter of less than about 100 nm, 110 nm, 120 nm, 130 nm, 140 nm, 150 nm, or 160 nm. In some embodiments, an LNP has a diameter of less than about 100 nm. In some embodiments, an LNP has a diameter of less than about 90 nm. In some embodiments, an LNP has a diameter of less than about 80 nm. In some embodiments, an LNP has a diameter of about 60-100 nm. In some embodiments, an LNP has a diameter of about 75-80 nm.

In some embodiments, the lipid nanoparticle compositions of the present disclosure are described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%.

In some embodiments, the mol-% of the phospholipid may be from about 1 mol-% to about 50 mol-%. In some embodiments, the mol-% of the phospholipid may be from about 2 mol-% to about 45 mol-%. In some embodiments, the mol-% of the phospholipid may be from about 3 mol-% to about 40 mol-%. In some embodiments, the mol-% of the phospholipid may be from about 4 mol-% to about 35 mol-%. In some embodiments, the mol-% of the phospholipid may be from about 5 mol-% to about 30 mol-%. In some embodiments, the mol-% of the phospholipid may be from about 10 mol-% to about 20 mol-%. In some embodiments, the mol-% of the phospholipid may be from about 5 mol-% to about 20 mol-%.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%.

In some embodiments, the mol-% of the PEG lipid may be from about 0.1 mol-% to about 10 mol-%. In some embodiments, the mol-% of the PEG lipid may be from about 0.2 mol-% to about 5 mol-%. In some embodiments, the mol-% of the PEG lipid may be from about 0.5 mol-% to about 3 mol-%. In some embodiments, the mol-% of the PEG lipid may be from about 1 mol-% to about 2 mol-%. In some embodiments, the mol-% of the PEG lipid may be about 1.5 mol-%. In some embodiments, the mol-% of the PEG lipid may be about 2.5 mol-%.

i. Ionizable Lipids

In some embodiments, an LNP disclosed herein comprises an ionizable lipid. In some embodiments, an LNP comprises two or more ionizable lipids.

Described below are a number of exemplary ionizable lipids of the present disclosure.

In some embodiments, an LNP of the present disclosure comprises an ionizable lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, an LNP of the present disclosure comprises an ionizable lipid disclosed in PCT Application PCT/US2022/076430.

### Formula (VII-A)

In some embodiments, Lipids of the Disclosure have a structure of Formula (VII-A):

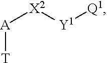

or a pharmaceutically acceptable salt thereof, wherein:
   A is —N(—X¹R¹)—, —C(R′)(-L¹-N(R″)R⁶)—, —C(R′)(—OR^{7a})—, —C(R′)(—N(R″)R^{8a})—, —C(R′)(—C(═O)OR^{9a})—, —C(R′)(—C(═O)N(R″)R^{10a})—, or —C(═N—R^{11a})—;
   T is —X^{2a}—Y^{1a}-Q^{1a} or —X³—C(═O)OR⁴;
   X¹ is optionally substituted C₂-C₆ alkylenyl;
   R¹ is —OH, —R^{1a},

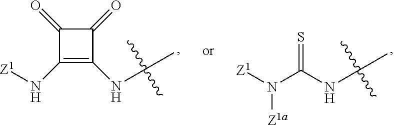

Z¹ is optionally substituted C₁-C₆ alkyl;
Z^{1a} is hydrogen or optionally substituted C₁-C₆ alkyl;
X² and X^{2a} are independently optionally substituted C₂-C₁₄ alkylenyl or optionally substituted C₂-C₁₄ alkenylenyl;
X³ is optionally substituted C₂-C₁₄ alkylenyl or optionally substituted C₂-C₁₄ alkenylenyl;
(i) Y¹ is

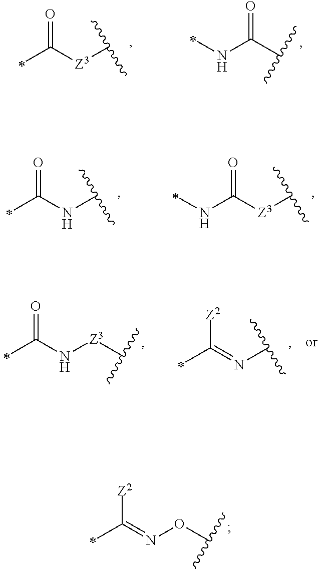

wherein the bond marked with an “*” is attached to X²;
   Y^{1a} is

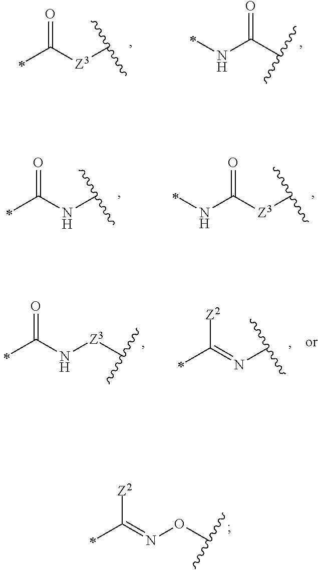

wherein the bond marked with an “*” is attached to X^{2a};
   each Z² is independently H or optionally substituted C₁-C₈ alkyl;
   each Z³ is independently optionally substituted C₁-C₆ alkylenyl;
   Q¹ is —NR²R³, —CH(OR²)(OR³), —CR²═C(R³)(R¹²), or —C(R²)(R³)(R¹²);
   Q^{1a} is —NR^{2′}R^{3′}, —CH(OR^{2′})(OR^{3′}), —CR²═C(R³)(R¹²), or —C(R^{2′})(R^{3′})(R^{12′}); or
   (ii) Y¹ is

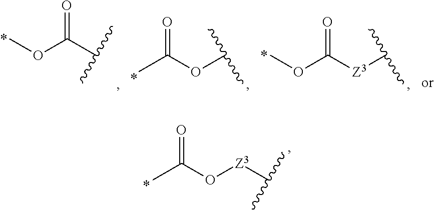

wherein the bond marked with an “*” is attached to X²;
   Y^{1a} is

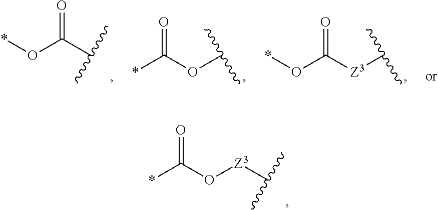

wherein the bond marked with an “*” is attached to X^{2a};
   each Z² is independently H or optionally substituted C₁-C₈ alkyl;
   each Z³ is independently optionally substituted C₁-C₆ alkylenyl;
   Q¹ is —NR²R³;
   Q^{1a} is —NR^{2′}R^{3′};
   R², R³, and R¹² are independently hydrogen, optionally substituted C₁-C₁₄ alkyl, optionally substituted C₂-C₁₄ alkenylenyl, or —(CH₂)ₘ-G-(CH₂)ₙH;
   R^{2′}, R^{3′}, and R^{12′} are independently hydrogen, optionally substituted C₁-C₁₄ alkyl, optionally substituted C₂-C₁₄ alkenylenyl, or —(CH₂)ₘ-G-(CH₂)ₙH;
   G is a C₃-C₈ cycloalkylenyl;
   each m is independently 0, 1, 2, 3, 4, 5, 6, 7, 8, 9, 10, 11, or 12;
   each n is independently 0, 1, 2, 3, 4, 5, 6, 7, 8, 9, 10, 11, or 12;
   X³ is optionally substituted C₂-C₁₄ alkylenyl;
   R⁴ is optionally substituted C₄-C₁₄ alkyl;
   L¹ is C₁-C₈ alkylenyl;
   R⁶ is C₁-C₆ alkyl, (hydroxy)C₁-C₆ alkyl, or (amino)C₁-C₆ alkyl
   R^{7a} is —C(═O)N(R′″)R^{7b}, —C(═S)N(R′″)R^{7b}, —N═C(R^{7b})(R^{7c}), or

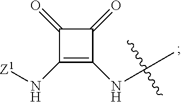

R^{7b} is C₁-C₆ alkyl, (hydroxy)C₁-C₆ alkyl, or (amino)C₁-C₆ alkyl;
R^{7c} is hydrogen or C₁-C₆ alkyl;
R^{8a} is —C(═O)N(R′″)R^{8b}, —C(═S)N(R′″)R^{8b}, —N═C(R^{8b})(R^{8c}), or

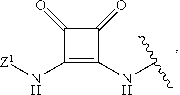

R^{8b} is C₁-C₆ alkyl, (hydroxy)C₁-C₆ alkyl, or (amino)C₁-C₆ alkyl;
R^{8c} is hydrogen or C₁-C₆ alkyl;
R^{9a} is —N═C(R^{9b})(R^{9c});
R^{9b} is C₁-C₆ alkyl, (hydroxy)C₁-C₆ alkyl, or (amino)C₁-C₆ alkyl;
R^{9c} is hydrogen or C₁-C₆ alkyl;
R^{10a} is —N═C(R^{10b})(R^{10c});
R^{10b} is C₁-C₆ alkyl, (hydroxy)C₁-C₆ alkyl, or (amino)C₁-C₆ alkyl;
R^{10c} is hydrogen or C₁-C₆ alkyl;
R^{11a} is —OR^{11b}, —N(R″)R^{11b}, —OC(═O)R^{11b}, or —N(R″)C(═O)R^{11b};
R^{11b} is C₁-C₆ alkyl, (hydroxy)C₁-C₆ alkyl, or (amino)C₁-C₆ alkyl;
R′ is hydrogen or C₁-C₆ alkyl;
R″ is hydrogen or C₁-C₆ alkyl; and
R′″ is hydrogen or C₁-C₆ alkyl.

### Formula (VIII-A)

In some embodiments, Lipids of the Disclosure have a structure of Formula (VII-A), wherein the Lipids of the Disclosure have a structure of Formula (VIII-A):

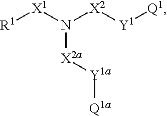

or a pharmaceutically acceptable salt thereof.

### Formula (IX-A)

In some embodiments, Lipids of the Disclosure have a structure of Formula (VII-A), wherein the Lipids of the Disclosure have a structure of Formula (IX-A):

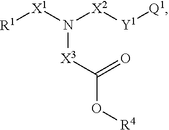

or a pharmaceutically acceptable salt thereof.

In some embodiments, Lipids of the Disclosure have a structure of Formula (VII-A), wherein A is —N(—X¹R¹)—.

In some embodiments, Lipids of the Disclosure have a structure of Formula (VII-A), wherein T is —X^{2a}—Y^{1a}-Q^{1a}.

In some embodiments, Lipids of the Disclosure have a structure of Formula (VII-A), wherein T is —X³—C(═O)OR⁴.

In some embodiments, Lipids of the Disclosure have a structure of Formula (VII-A), (VIII-A), or (IX-A), wherein X² and/or X^{2a} are/is optionally substituted C₂-C₁₄ alkylenyl (e.g., C₄-C₁₀ alkylenyl, C₅-C₇ alkylenyl, C₅, C₆, or C₇ alkylenyl). In some embodiments, Lipids of the Disclosure have a structure of Formula (VII-A), (VIII-A), or (IX-A), wherein X² is C₄-C₁₀ alkylenyl. In some embodiments, Lipids of the Disclosure have a structure of Formula (VII-A), (VIII-A), or (IX-A), wherein X^{2a} is C₄-C₁₀ alkylenyl. In some embodiments, Lipids of the Disclosure have a structure of Formula (VII-A), (VIII-A), or (IX-A), wherein X² is C₅ alkylenyl. In some embodiments, Lipids of the Disclosure have a structure of Formula (VII-A), (VIII-A), or (IX-A), wherein X² is C₆ alkylenyl. In some embodiments, Lipids of the Disclosure have a structure of Formula (VII-A), (VIII-A), or (IX-A), wherein X^{2a} is C₅ alkylenyl. In some embodiments, Lipids of the Disclosure have a structure of Formula Formula (VII-A), (VIII-A), or (IX-A), wherein X^{2a} is C₆ alkylenyl.

In some embodiments, Lipids of the Disclosure have a structure of Formula (VII-A), (VIII-A), or (IX-A), wherein Y¹ and/or Y^{1a} are/is

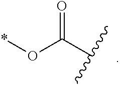 [SMILES: *OC(*)=O]

In some embodiments, Lipids of the Disclosure have a structure of Formula (VII-A), (VIII-A), or (IX-A), wherein Y¹ is

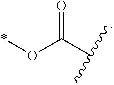 [SMILES: *OC(*)=O]

In some embodiments, Lipids of the Disclosure have a structure of Formula (VII-A), (VIII-A), or (IX-A), wherein Y^{1a} is

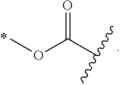 [SMILES: *C(=O)OC]

In some embodiments, Lipids of the Disclosure have a structure of Formula (VII-A), (VIII-A), or (IX-A), wherein Y¹ and/or Y^{1a} are/is

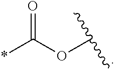 [SMILES: *OC(*)=O]

In some embodiments, Lipids of the Disclosure have a structure of Formula (VII-A), (VIII-A), or (IX-A), wherein Y¹ is

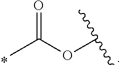 [SMILES: *OC(C)=O]

In some embodiments, Lipids of the Disclosure have a structure of Formula (VII-A), (VIII-A), or (IX-A), wherein Y^{1a} is

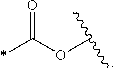 [SMILES: *OC(*)=O]

In some embodiments, Lipids of the Disclosure have a structure of Formula (VII-A), (VIII-A), or (IX-A), wherein Y¹ and/or Y^{1a} are/is

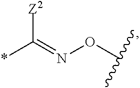

wherein Z² is hydrogen.

In some embodiments, Lipids of the Disclosure have a structure of Formula (VII-A), (VIII-A), or (IX-A), wherein Y¹ is

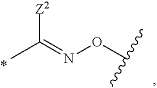

wherein Z² is hydrogen.

In some embodiments, Lipids of the Disclosure have a structure of Formula (VII-A), (VIII-A), or (IX-A), wherein Y^{1a} is

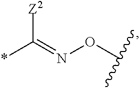

wherein Z² is hydrogen.

In some embodiments, Lipids of the Disclosure have a structure of Formula (VII-A), (VIII-A), or (IX-A), wherein Y¹ and/or Y^{1a} are/is

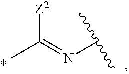

wherein Z² is hydrogen.

In some embodiments, Lipids of the Disclosure have a structure of Formula (VII-A), (VIII-A), or (IX-A), wherein Y¹ is

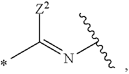

wherein Z² is hydrogen.

In some embodiments, Lipids of the Disclosure have a structure of Formula (VII-A), (VILI-A), or (IX-A), wherein Y^{1a} is

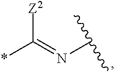

wherein Z² is hydrogen.

In some embodiments, Lipids of the Disclosure have a structure of Formula (VII-A), (VIII-A), or (IX-A), wherein Y¹ and Y^{1a} are independently

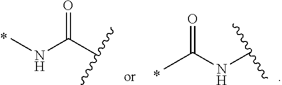

In some embodiments, Lipids of the Disclosure have a structure of Formula (VII-A), (VIII-A), or (IX-A), wherein Y¹ is independently

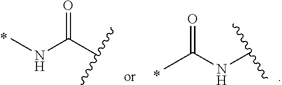

In some embodiments, Lipids of the Disclosure have a structure of Formula (VII-A), VIII-A), or (IX-A), wherein Y^{1a} is independently

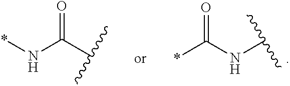 [SMILES: *NC(*)=O ; *NC(*)=O]

In some embodiments, Lipids of the Disclosure have a structure of Formula (VII-A), (VIII-A), or (IX-A), wherein Q¹ and/or Q^{1a} are/is —NR²R³. In some embodiments, Lipids of the Disclosure have a structure of Formula (VII-A), (VIII-A), or (IX-A), wherein Q¹ is —NR²R³. In some embodiments, Lipids of the Disclosure have a structure of Formula (VII-A), (VIII-A), or (IX-A), wherein Q^{1a} is —NR²R³.

In some embodiments, Lipids of the Disclosure have a structure of Formula (VII-A), (VIII-A), or (IX-A), wherein Q¹ and/or Q^{1a} are/is —CH(OR²)(OR³). In some embodiments, Lipids of the Disclosure have a structure of Formula (VII-A), (VIII-A), or (IX-A), wherein Q¹ is —CH(OR²)(OR³). In some embodiments, Lipids of the Disclosure have a structure of Formula (VII-A), (VIII-A), or (IX-A), wherein Q^{3a} is —CH(OR²)(OR³).

In some embodiments, Lipids of the Disclosure have a structure of Formula (VII-A), (VIII-A), or (IX-A), wherein Q¹ and/or Q^{1a} are/is —CR²═C(R³)(R¹²). In some embodiments, Lipids of the Disclosure have a structure of Formula (VII-A), (VIII-A), or (IX-A), wherein Q¹ is —CR²═C(R³)(R¹²). In some embodiments, Lipids of the Disclosure have a structure of Formula (VII-A), (VIII-A), or (IX-A), wherein Q^{1a} is —CR²═C(R³)(R²).

In some embodiments, Lipids of the Disclosure have a structure of Formula (VII-A), (VIII-A), or (IX-A), wherein Q¹ and/or Q^{1a} are/is —C(R^{2′})(R^{3′})(R^{12′}). In some embodiments, Lipids of the Disclosure have a structure of Formula (VII-A), (VIII-A), or (IX-A), wherein Q¹ is —C(R^{2′})(R^{3′})(R¹²). In some embodiments, Lipids of the Disclosure have a structure of Formula (VII-A), (VIII-A), or (IX-A), wherein Q^{1a} is —C(R^{2′})(R^{3′})(R^{12′}).

In some embodiments, Lipids of the Disclosure have a structure of Formula (VII-A), (VIII-A), or (IX-A), wherein X³ is optionally substituted C₂-C₁₄ alkylenyl (e.g., C₄-C₁₀ alkylenyl, C₅-C₇ alkylenyl, C₅, C₆, or C₇ alkylenyl). In some embodiments, Lipids of the Disclosure have a structure of Formula (VII-A), (VIII-A), or (IX-A), wherein X³ is C₅-C₇ alkylenyl. In some embodiments, Lipids of the Disclosure have a structure of Formula (VII-A), (VIII-A), or (IX-A), wherein X³ is C₅ alkylenyl.

In some embodiments, Lipids of the Disclosure have a structure of Formula (VII-A), (VIII-A), or (IX-A), wherein R², R³, R¹², R^{2′}, R^{3′}, and/or R^{12′} are hydrogen. In some embodiments, Lipids of the Disclosure have a structure of Formula (VII-A), (VIII-A), or (IX-A), wherein R² is hydrogen. In some embodiments, Lipids of the Disclosure have a structure of Formula (VII-A), (VIII-A), or (IX-A), wherein R³, is hydrogen. In some embodiments, Lipids of the Disclosure have a structure of Formula (VII-A), (VIII-A), or (IX-A), wherein R¹² is hydrogen. In some embodiments, Lipids of the Disclosure have a structure of Formula (VII-A), (VIII-A), or (IX-A), wherein R^{2′} is hydrogen. In some embodiments, Lipids of the Disclosure have a structure of Formula (VII-A), (VIII-A), or (IX-A), wherein R^{3′} is hydrogen. In some embodiments, Lipids of the Disclosure have a structure of Formula (VII-A), (VIII-A), or (IX-A), wherein R^{12′} is hydrogen.

In some embodiments, Lipids of the Disclosure have a structure of Formula (VII-A), (VIII-A), or (IX-A), wherein R², R³, R¹², R^{2′}, R^{3′}, and/or R^{12′} are optionally substituted C₁-C₁₄ alkyl (e.g., C₅-C₁₄, C₅-C₁₀, C₆-C₉, C₅, C₆, C₇, C₈, C₉, C₁₀ alkyl). In some embodiments, Lipids of the Disclosure have a structure of Formula (VII-A), (VIII-A), or (IX-A), wherein R² is C₅-C₁₀ alkyl. In some embodiments, Lipids of the Disclosure have a structure of Formula (VII-A), (VIII-A), or (IX-A), wherein R³ is C₅-C₁₀ alkyl. In some embodiments, Lipids of the Disclosure have a structure of Formula (VII-A), (VIII-A), or (IX-A), wherein R¹² is C₅-C₁₀ alkyl. In some embodiments, Lipids of the Disclosure have a structure of Formula (VII-A), (VIII-A), or (IX-A), wherein R² is C₅-C₁₀ alkyl. In some embodiments, Lipids of the Disclosure have a structure of Formula (VII-A), (VIII-A), or (IX-A), wherein R^{3′} is C₅-C₁₀ alkyl. In some embodiments, Lipids of the Disclosure have a structure of Formula (VII-A), (VIII-A), or (IX-A), wherein R^{12′} is C₅-C₁₀ alkyl. In some embodiments, Lipids of the Disclosure have a structure of Formula (VII-A), (VIII-A), or (IX-A), wherein R² is C₈ alkyl. In some embodiments, Lipids of the Disclosure have a structure of Formula (VII-A), (VIII-A), or (IX-A), wherein R³ is C₈ alkyl. In some embodiments, Lipids of the Disclosure have a structure of Formula (VII-A), (VIII-A), or (IX-A), wherein R¹² is C₈ alkyl. In some embodiments, Lipids of the Disclosure have a structure of Formula (VII-A), (VIII-A), or (IX-A), wherein R^{2′} is C₈ alkyl. In some embodiments, Lipids of the Disclosure have a structure of Formula (VII-A), (VIII-A), or (IX-A), wherein R^{3′} is C₈ alkyl. In some embodiments, Lipids of the Disclosure have a structure of Formula (VII-A), (VIII-A), or (IX-A), wherein R^{2′} is C₈ alkyl.

In some embodiments, Lipids of the Disclosure have a structure of Formula (VII-A) or (IX-A), wherein R⁴ is optionally substituted C₄-C₁₄ alkyl (e.g., C₆-C₁₂, C₅-C₁₂, C₆, C₇, C₈, C₉, C₁₀, C₁₁, C₁₂ alkyl). In some embodiments, Lipids of the Disclosure have a structure of Formula (VII-A) or (IX-A), wherein R⁴ is C₆-C₁₂ alkyl. In some embodiments, Lipids of the Disclosure have a structure of Formula (VII-A), (VIII-A), or (IX-A), wherein R⁴ is C₁₁ alkyl.

In some embodiments, Lipids of the Disclosure have a structure of Formula (VII-A), (VIII-A), or (IX-A), wherein R¹ is OH.

In some embodiments, Lipids of the Disclosure have a structure of Formula (VII-A), (VIII-A), or (IX-A), wherein X¹ is C₂₋₄ alkylenyl (e.g., C₂, C₃, or C₄ alkylenyl). In some embodiments, Lipids of the Disclosure have a structure of Formula (VII-A), (VIII-A), or (IX-A), wherein X¹ is C₂ alkylenyl. In some embodiments, Lipids of the Disclosure have a structure of Formula (VII-A), (VIII-A), or (IX-A), wherein X¹ is C₄ alkylenyl.

### Formula (VII-B)

In some embodiments, Lipids of the Disclosure have a structure of Formula (VII-B):

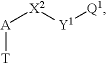

or a pharmaceutically acceptable salt thereof, wherein:
   A is —C(R′)(-L¹-N(R″)R⁶)—, —C(R′)(—OR^{7a})—, —C(R′)(—N(R″)R^{8a})—, —C(R′)(—C(═O)OR^{9a})—, —C(R′)(—C(═O)N(R″)R^{10a})—, or —C(═N—R^{11a})—;
   T is —X^{2a}—Y^{1a}-Q^{1a} or —X³—C(═O)OR⁴;
   X² and X^{2a} are independently optionally substituted C₂-C₁₄ alkylenyl or optionally substituted C₂-C₁₄ alkenylenyl;
   X³ is optionally substituted C₁-C₁₄ alkylenyl or optionally substituted C₂-C₁₄ alkenylenyl;
   Y¹ is

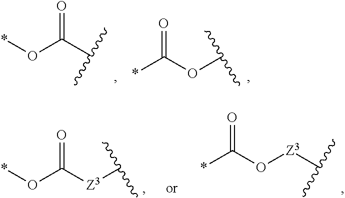

wherein the bond marked with an “*” is attached to X²;
   Y^{1a} is

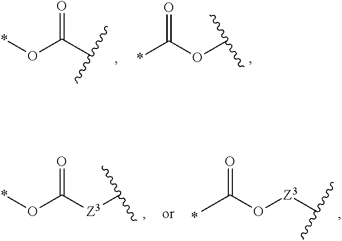

wherein the bond marked with an “*” is attached to X^{2a};
   each Z³ is independently optionally substituted C₁-C₆ alkylenyl or optionally substituted C₂-C₁₄ alkenylenyl;
   Q¹ is —NR²R³, —CH(OR²)(OR³), —CR²═C(R³)(R¹²), or —C(R²)(R³)(R¹²);
   Q^{1a} is —NR^{2′}R^{3′}, —CH(OR^{2′})(OR^{3′}), —CR²═C(R³)(R¹²), or —C(R^{2′})(R^{3′})(R^{12′});
   R², R³, and R¹² are independently hydrogen, optionally substituted C₁-C₁₄ alkyl, optionally substituted C₂-C₁₄ alkenylenyl, or —(CH₂)ₘ-G-(CH₂)ₙH;
   R^{2′}, R^{3′}, and R^{12′} are independently hydrogen, optionally substituted C₁-C₁₄ alkyl, optionally substituted C₂-C₁₄ alkenylenyl, or —(CH₂)ₘ-G-(CH₂)ₙH;
   G is a C₃-C₈ cycloalkylenyl;
   each m is independently 0, 1, 2, 3, 4, 5, 6, 7, 8, 9, 10, 11, or 12;
   each n is independently 0, 1, 2, 3, 4, 5, 6, 7, 8, 9, 10, 11, or 12;
   X³ is optionally substituted C₂-C₁₄ alkylenyl;
   R⁴ is optionally substituted C₄-C₁₄ alkyl;
   L¹ is C₁-C₈ alkylenyl;
   R⁶ is (hydroxy)C₁-C₆ alkyl, or (amino)C₁-C₆ alkyl.
   R^{7a} is —C(═O)N(R′″)R^{7b}, —C(═S)N(R′″)R^{7b}, —N═C(R^{7b})(R^{7c}),

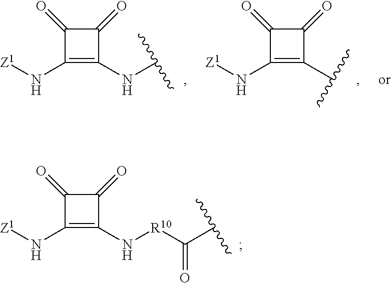

Z¹ is optionally substituted C₁-C₆ alkyl;
R¹⁰ is C₁-C₆ alkylenyl;
R^{7b} is C₁-C₆ alkyl, (hydroxy)C₁-C₆ alkyl, or (amino)C₁-C₆ alkyl;
R^{7c} is hydrogen or C₁-C₆ alkyl;
R^{8a} is —C(═O)N(R′″)R^{8b}, —C(═S)N(R′″)R^{8b}, —N═C(R^{8b})(R^{8c}),

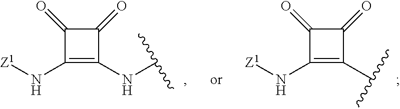

R^{8b} is C₁-C₆ alkyl, (hydroxy)C₁-C₆ alkyl, or (amino)C₁-C₆ alkyl;
R^{8c} is hydrogen or C₁-C₆ alkyl;
R^{9a} is —N═C(R^{9b})(R^{9c});
R^{9b} is C₁-C₆ alkyl, (hydroxy)C₁-C₆ alkyl, or (amino)C₁-C₆ alkyl;
R^{9c} is hydrogen or C₁-C₆ alkyl;
R^{10a} is —N═C(R^{10b})(R^{10c});
R^{10b} is C₁-C₆ alkyl, (hydroxy)C₁-C₆ alkyl, or (amino)C₁-C₆ alkyl;
R^{10c} is hydrogen or C₁-C₆ alkyl;
R^{11a} is —OR^{11b}, —N(R″)R^{11b}, —OC(═O)R^{11b}, or —N(R″)C(═O)R^{11b};
R^{11b} is C₁-C₆ alkyl, (hydroxy)C₁-C₆ alkyl, or (amino)C₁-C₆ alkyl;
R′ is hydrogen or C₁-C₆ alkyl;
R″ is hydrogen or C₁-C₆ alkyl; and
R′″ is hydrogen or C₁-C₆ alkyl.

In some embodiments, Lipids of the Disclosure have a structure of Formula (VII-B), wherein A is —C(R′)(-L¹-N(R″)R⁶)—.

In some embodiments, Lipids of the Disclosure have a structure of Formula (VII-B), wherein A is —C(R′)(—OR^{7a})—.

In some embodiments, Lipids of the Disclosure have a structure of Formula (VII-B), wherein A is —C(R′)(—N(R″)R^{8a}).

In some embodiments, Lipids of the Disclosure have a structure of Formula (VII-B), wherein A is —C(R′)(—C(═O)OR^{9a})

In some embodiments, Lipids of the Disclosure have a structure of Formula (VII-B), wherein A is —C(R′)(—C(═O)N(R″)R^{10a})—.

In some embodiments, Lipids of the Disclosure have a structure of Formula (VII-B), wherein A is —C(═N—R^{11a})—.

In some embodiments, Lipids of the Disclosure have a structure of Formula (VII-B), wherein T is —X^{2a}—Y^{1a}-Q^{1a}.

In some embodiments, Lipids of the Disclosure have a structure of Formula (VII-B), wherein T is —X³—C(═O)OR⁴.

In some embodiments, Lipids of the Disclosure have a structure of Formula (VII-B), wherein X² and/or X^{2a} are/is optionally substituted C₂-C₁₄ alkylenyl (e.g., C₂-C₁₀ alkylenyl, C₂-C₈ alkylenyl, C₂, C₃, C₄, C₅, C₆, C₇, or C₈ alkylenyl). In some embodiments, Lipids of the Disclosure have a structure of Formula (VII-B), wherein X² is C₂-C₁₄ alkylenyl. In some embodiments, Lipids of the Disclosure have a structure of Formula (VII-B), wherein X^{2a} is C₂-C₁₄ alkylenyl

In some embodiments, Lipids of the Disclosure have a structure of Formula (VII-B), wherein Y³ and/or Y^{1a} are/is

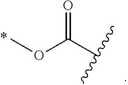 [SMILES: *C(=O)OC]

In some embodiments, Lipids of the Disclosure have a structure of Formula (VII-B), wherein Y¹ is

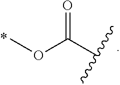 [SMILES: *C(=O)OC]

In some embodiments, Lipids of the Disclosure have a structure of Formula (VII-B), wherein Y^{1a} is

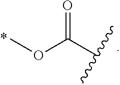 [SMILES: *C(=O)OC]

In some embodiments, Lipids of the Disclosure have a structure of Formula (VII-B), wherein Y¹ and/or Y^{1a} are/is

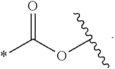 [SMILES: *OC(*)=O]

In some embodiments, Lipids of the Disclosure have a structure of Formula (VII-B), wherein Y¹ is

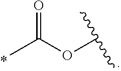

In some embodiments, Lipids of the Disclosure have a structure of Formula (VII-B), wherein Y^{1a} is

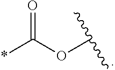 [SMILES: *OC(*)=O]

In some embodiments, Lipids of the Disclosure have a structure of Formula (VII-B), wherein Y¹ and/or Y^{1a} are/is

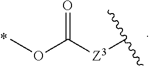

In some embodiments, Lipids of the Disclosure have a structure of Formula (VII-B), wherein Y¹ is

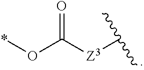

In some embodiments, Lipids of the Disclosure have a structure of Formula (VII-B), wherein Y^{1a} is

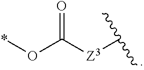

In some embodiments, Lipids of the Disclosure have a structure of Formula (VII-B), wherein Y¹ and/or Y^{1a} are/is

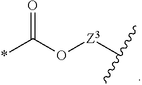

In some embodiments, Lipids of the Disclosure have a structure of Formula (VII-B), wherein Y¹ is

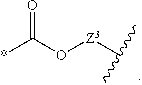

In some embodiments, Lipids of the Disclosure have a structure of Formula (VII-B), wherein Y^{1a} is

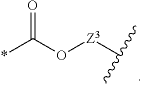

In some embodiments, Lipids of the Disclosure have a structure of Formula (VII-B), wherein Q¹ and/or Q^{1a} are/is —C(R^{2′})(R^{3′})(R^{12′}). In some embodiments, Lipids of the Disclosure have a structure of Formula (VII-B), wherein Q¹ is —C(R^{2′})(R³)(R^{12′}). In some embodiments, Lipids of the Disclosure have a structure of Formula (VII-B), wherein Q^{1a} is —C(R^{2′})(R^{3′})(R^{12′}).

In some embodiments, Lipids of the Disclosure have a structure of Formula (VII-B), wherein X³ is optionally substituted C₁-C₁₄ alkylenyl (e.g., C₁-C₆, C₁-C₄ alkylenyl). In some embodiments, Lipids of the Disclosure have a structure of Formula (VII-B), wherein X³ is C₁-C₁₄ alkylenyl.

In some embodiments, Lipids of the Disclosure have a structure of Formula (VII-B), wherein R², R³, R¹², R^{2′}, R^{3′}, and/or R^{12′} are hydrogen. In some embodiments, Lipids of the Disclosure have a structure of Formula (VII-B), wherein R² is hydrogen. In some embodiments, Lipids of the Disclosure have a structure of Formula (VII-B), wherein R³ is hydrogen. In some embodiments, Lipids of the Disclosure have a structure of Formula (VII-B), wherein R¹² is hydrogen. In some embodiments, Lipids of the Disclosure have a structure of Formula (VII-B), wherein R^{2′} is hydrogen. In some embodiments, Lipids of the Disclosure have a structure of Formula (VII-B), wherein R^{3′} is hydrogen. In some embodiments, Lipids of the Disclosure have a structure of Formula (VII-B), wherein R^{12′} is hydrogen.

In some embodiments, Lipids of the Disclosure have a structure of Formula (VII-B), wherein R², R³, R¹², R^{2′}, R^{3′}, and/or R^{12′} are optionally substituted C₁-C₁₄ alkyl (e.g., C₄-C₁₀ alkyl, C₅, C₆, C₇, C₈, C₉ alkyl). In some embodiments, Lipids of the Disclosure have a structure of Formula (VII-B), wherein R² is C₄-C₁₀ alkyl. In some embodiments, Lipids of the Disclosure have a structure of Formula (VII-B), wherein R³ is C₄-C₁₀ alkyl. In some embodiments, Lipids of the Disclosure have a structure of Formula (VII-B), wherein R¹² is C₄-C₁₀ alkyl. In some embodiments, Lipids of the Disclosure have a structure of Formula (VII-B), wherein R^{2′} is C₄-C₁₀ alkyl. In some embodiments, Lipids of the Disclosure have a structure of Formula (VII-B), wherein R^{3′} is C₄-C₁₀ alkyl. In some embodiments, Lipids of the Disclosure have a structure of Formula (VII-B), wherein R^{12′} is C₄-C₁₀ alkyl.

In some embodiments, Lipids of the Disclosure have a structure of Formula (VII-B), wherein R⁴ is optionally substituted C₄-C₁₄ alkyl (e.g., C₈-C₁₄ alkyl, linear C₅-C₁₄ alkyl, C₈, C₉, C₁₀, C₁₁, C₁₂, C₁₃, or C₁₄ alkyl). In some embodiments, Lipids of the Disclosure have a structure of Formula (VII-B), wherein R⁴ is linear C₅-C₁₄ alkyl. In some embodiments, Lipids of the Disclosure have a structure of Formula (VII-B), wherein R⁴ is linear C₁₁ alkyl.

In some embodiments, Lipids of the Disclosure have a structure of Formula (VII-B), wherein L¹ is C₁-C₃ alkylenyl.

In some embodiments, Lipids of the Disclosure have a structure of Formula (VII-B), wherein R⁶ is (hydroxy)C₁-C₆ alkyl.

In some embodiments, Lipids of the Disclosure have a structure of Formula (VII-B), wherein R^{7a} is

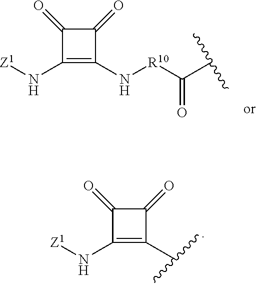

In some embodiments, Lipids of the Disclosure have a structure of Formula (VII-B), wherein R^{7a} is

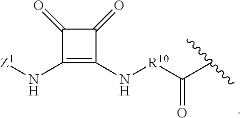

In some embodiments, Lipids of the Disclosure have a structure of Formula (VII-B), wherein R^{7a} is

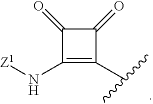

In some embodiments, Lipids of the Disclosure have a structure of Formula (VII-B), wherein R^{7a} is selected from the group consisting of —C(═O)N(R′″)R^{7b}, —C(═S)N(R′″)R^{7b}, and —N═C(R^{7b})(R^{7c}). In some embodiments, Lipids of the Disclosure have a structure of Formula (VII-B), wherein R^{7a} is —C(═O)N(R′″)R^{7b}. In some embodiments, Lipids of the Disclosure have a structure of Formula (VII-B), wherein R^{7a} is —C(═S)N(R′″)R^{7b}. In some embodiments, Lipids of the Disclosure have a structure of Formula (VII-B), wherein R^{7a} is —N═C(R^{7b})(R^{7c}).

In some embodiments, Lipids of the Disclosure have a structure of Formula (VII-B), wherein R^{8a} is selected from the group consisting of —C(═O)N(R′″)R^{8b}, —C(═S)N(R′″)R^{7b}, and —N═C(R^{8b})(R^{8c}). In some embodiments, Lipids of the Disclosure have a structure of Formula (VII-B), wherein R^{8a} is —C(═O)N(R′″)R^{8b}. In some embodiments, Lipids of the Disclosure have a structure of Formula (VII-B), wherein R^{8a} is —C(═S)N(R′″)R^{8b}. In some embodiments, Lipids of the Disclosure have a structure of Formula (VII-B), wherein R^{8a} is —N═C(R^{8b})(R^{8c}).

In some embodiments, Lipids of the Disclosure have a structure of Formula (VII-B), wherein R^{8a} is

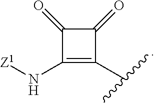

In some embodiments, Lipids of the Disclosure have a structure of Formula (VII-B), wherein R^{9b} is (hydroxy)C₁-C₆ alkyl.

In some embodiments, Lipids of the Disclosure have a structure of Formula (VII-B), wherein R^{10b} is (amino)C₁-C₆ alkyl.

In some embodiments, Lipids of the Disclosure have a structure of Formula (VII-B), wherein R^{11a} is —OR^{11b} or —OC(═O)R^{11b}. In some embodiments, Lipids of the Disclosure have a structure of Formula (VII-B), wherein R^{11a} is —OR^{11b}. In some embodiments, Lipids of the Disclosure have a structure of Formula (VII-B), wherein R^{11a} is —OC(═O)R^{11b}.

In some embodiments, Lipids of the Disclosure have a structure of Formula (VII-B), wherein R^{11a} is —N(R″)R^{11b} or —N(R″)C(═O)R^{11b}. In some embodiments, Lipids of the Disclosure have a structure of Formula (VII-B), wherein R^{11a} is —N(R″)R^{11b}. In some embodiments, Lipids of the Disclosure have a structure of Formula (VII-B), wherein R^{11a} is —N(R″)C(═O)R^{11b}.

In some embodiments, Lipids of the Disclosure have a structure of Formula (VII-B), wherein R^{11b} is (amino)C₁-C₆ alkyl.

### Formula (VII-C)

In some embodiments, Lipids of the Disclosure have a structure of Formula (VII-C):

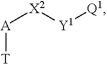

or a pharmaceutically acceptable salt thereof, wherein:
   A is —N(—X¹R¹)—;
   T is —X^{2a}—Y^{1a}-Q^{1a} or —X³—C(═O)OR⁴;
   (i) X¹ is optionally substituted C₂-C₃ alkylenyl;
   R¹ is

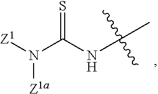

NR″C(O)OR²⁰, or —NR″R²¹; or
(ii) X¹ is C₄-C₆alkylenyl, and
R¹ is

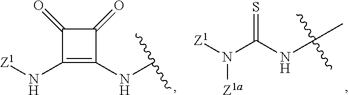

—NR″C(O)OR²⁰, or —NR″R²¹;
Z¹ is optionally substituted C₁-C₆ alkyl;
Z^{1a} is hydrogen or optionally substituted C₁-C₆ alkyl;
R²⁰ is optionally substituted C₁-C₆alkyl;
R²¹ is —(C₂ alkylenyl)-OH;
X² and X^{2a} are independently optionally substituted C₂-C₁₄ alkylenyl or optionally substituted C₂-C₁₄ alkenylenyl;
X³ is optionally substituted C₂-C₁₄ alkylenyl or optionally substituted C₂-C₁₄ alkenylenyl;
Y¹ is a bond,

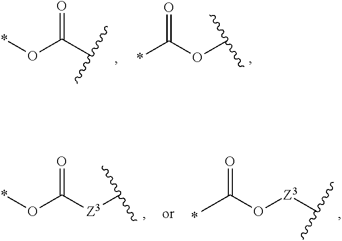

wherein the bond marked with an “*” is attached to X²;
   Y^{1a} is

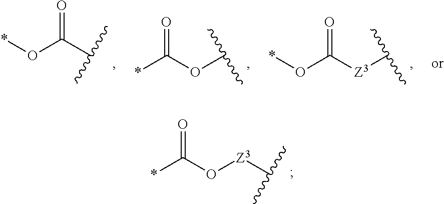

wherein the bond marked with an “*” is attached to X^{2a};
wherein Y¹ and Y^{1a} are

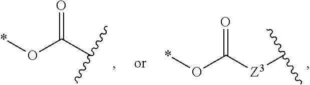

when R¹ is

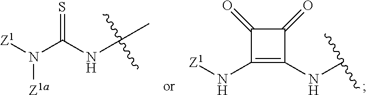

each Z² is independently H or optionally substituted C₁-C₈ alkyl;
each Z³ is independently optionally substituted C₁-C₆ alkylenyl or optionally substituted C₂-C₁₄ alkenylenyl;
Q¹ is —NR²R³, —CH(OR²)(OR³), —CR²═C(R³)(R¹²), or —C(R²)(R³)(R¹²);
Q^{1a} is —NR^{2′}R^{3′}, —CH(OR^{2′})(OR^{3′}), —CR²═C(R³)(R¹²), or —C(R^{2′})(R^{3′})(R^{12′}); wherein Q¹ is —CH(OR²)(OR³) and Q^{1a} is —CH(OR^{2′})(OR^{3′}) when R¹ is —NR″C(O)OR²⁰;
R², R³, and R¹² are independently hydrogen, optionally substituted linear C₁-C₁₄ alkyl, optionally substituted C₂-C₁₄ alkenylenyl, or —(CH₂)ₘ-G-(CH₂)ₙH;
R^{2′}, R^{3′}, and R^{12′} are independently hydrogen, optionally substituted linear C₁-C₁₄ alkyl, or optionally substituted C₂-C₁₄ alkenylenyl;
X³ is optionally substituted C₂-C₁₄ alkylenyl;
R⁴ is optionally substituted C₄-C₁₄ alkyl; and
R″ is hydrogen or C₁-C₆ alkyl.

In some embodiments, Lipids of the Disclosure have a structure of Formula (VII-C), wherein R¹ is

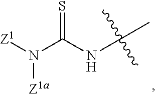

wherein Z¹ is methyl and Z^{1a} is hydrogen or methyl.

In some embodiments, Lipids of the Disclosure have a structure of Formula (VII-C), wherein R¹ is

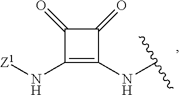

wherein Z¹ is methyl.

In some embodiments, Lipids of the Disclosure have a structure of Formula (VII-C), wherein R¹ is —NR″C(O)OR²⁰.

In some embodiments, Lipids of the Disclosure have a structure of Formula (VII-C), wherein R¹ is —NR″R²¹.

In some embodiments, Lipids of the Disclosure have a structure of Formula (VII-C), wherein R²⁰ is t-butyl or benzyl.

In some embodiments, Lipids of the Disclosure have a structure of Formula (VII-C), wherein X² and/or X^{2a} are/is optionally substituted C₂-C₁₄ alkylenyl (e.g., C₄-C₈ alkylenyl, C₄, C₅, C₆, C₇, C₈ alkylenyl). In some embodiments, Lipids of the Disclosure have a structure of Formula (VII-C), wherein X² is C₄-C₈ alkylenyl. In some embodiments, Lipids of the Disclosure have a structure of Formula (VII-C), wherein X^{2a} is C₄-C₈ alkylenyl.

In some embodiments, Lipids of the Disclosure have a structure of Formula (VII-C), wherein Y¹ and/or Y^{1a} are/is

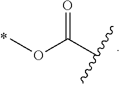 [SMILES: *C(=O)OC]

In some embodiments, Lipids of the Disclosure have a structure of Formula (VII-C), wherein Y¹ is

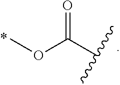 [SMILES: *C(=O)OC]

In some embodiments, Lipids of the Disclosure have a structure of Formula (VII-C), wherein Y^{1a} is

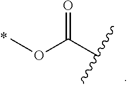 [SMILES: *C(=O)OC]

In some embodiments, Lipids of the Disclosure have a structure of Formula (VII-C), wherein Y¹ and/or Y^{1a} are/is

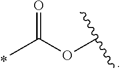

In some embodiments, Lipids of the Disclosure have a structure of Formula (VII-C), wherein Y¹ is

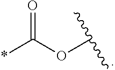 [SMILES: *OC(*)=O]

In some embodiments, Lipids of the Disclosure have a structure of Formula (VII-C), wherein Y^{1a} is

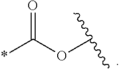 [SMILES: *OC(*)=O]

In some embodiments, Lipids of the Disclosure have a structure of Formula (VII-C), wherein Y¹ and/or Y^{1a} are/is

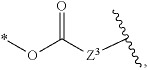

wherein Z³ is C₂ alkylenyl.

In some embodiments, Lipids of the Disclosure have a structure of Formula (VII-C), wherein Y¹ is

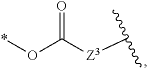

wherein Z³ is C₂ alkylenyl.

In some embodiments, Lipids of the Disclosure have a structure of Formula (VII-C), wherein Y^{1a} is

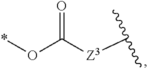

wherein Z³ is C₂ alkylenyl.

In some embodiments, Lipids of the Disclosure have a structure of Formula (VII-C), wherein Y¹ and/or Y^{1a} are/is

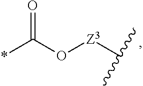

wherein Z³ is C₂ alkylenyl.

In some embodiments, Lipids of the Disclosure have a structure of Formula (VII-C), wherein Y¹ is

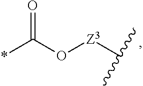

wherein Z³ is C₂ alkylenyl.

In some embodiments, Lipids of the Disclosure have a structure of Formula (VII-C), wherein Y^{1a} is

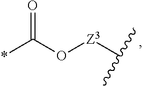

wherein Z³ is C₂ alkylenyl.

In some embodiments, Lipids of the Disclosure have a structure of Formula (VII-C), wherein Q¹ and/or Q^{1a} are/is —CH(OR²)(OR³). In some embodiments, Lipids of the Disclosure have a structure of Formula (VII-C), wherein Q^{1a} is —CH(OR²)(OR³). In some embodiments, Lipids of the Disclosure have a structure of Formula (VII-C), wherein Q¹ is —CH(OR²)(OR³).

In some embodiments, Lipids of the Disclosure have a structure of Formula (VII-C), wherein Q¹ and/or Q^{M} are/is —C(R^{2′})(R^{3′})(R^{12′}). In some embodiments, Lipids of the Disclosure have a structure of Formula (VII-C), wherein Q¹ is —C(R^{2′})(R^{3′})(R^{12′}). In some embodiments, Lipids of the Disclosure have a structure of Formula (VII-C), wherein Q^{1a} is —C(R^{2′})(R^{3′})(R^{12′}).

In some embodiments, Lipids of the Disclosure have a structure of Formula (VII-C), wherein R², R³, R¹², R^{2′}, R^{3′}, and R^{12′} are independently hydrogen, optionally substituted linear C₁-C₁₄ alkyl (e.g., C₄-C₁₀alkyl, C₆-C₈ alkyl, C₅, C₆, C₇, C₈, C₉ alkyl). In some embodiments, Lipids of the Disclosure have a structure of Formula (VII-C), wherein R² is hydrogen. In some embodiments, Lipids of the Disclosure have a structure of Formula (VII-C), wherein R³ is hydrogen. In some embodiments, Lipids of the Disclosure have a structure of Formula (VII-C), wherein R¹² is hydrogen. In some embodiments, Lipids of the Disclosure have a structure of Formula (VII-C), wherein R^{2′} is hydrogen. In some embodiments, Lipids of the Disclosure have a structure of Formula (VII-C), wherein R^{3′} is hydrogen. In some embodiments, Lipids of the Disclosure have a structure of Formula (VII-C), wherein R^{12′} is hydrogen. In some embodiments, Lipids of the Disclosure have a structure of Formula (VII-C), wherein R² is linear C₄-C₁₀alkyl. In some embodiments, Lipids of the Disclosure have a structure of Formula (VII-C), wherein R³ is linear C₄-C₁₀alkyl. In some embodiments, Lipids of the Disclosure have a structure of Formula (VII-C), wherein R¹² is linear C₄-C₁₀alkyl. In some embodiments, Lipids of the Disclosure have a structure of Formula (VII-C), wherein R^{2′} is linear C₄-C₁₀alkyl. In some embodiments, Lipids of the Disclosure have a structure of Formula (VII-C), wherein R^{3′} is linear C₄-C₁₀alkyl. In some embodiments, Lipids of the Disclosure have a structure of Formula (VII-C), wherein R^{12′} is linear C₄-C₁₀alkyl.

### Formula (I-A)

In some embodiments, Lipids of the Disclosure have a structure of Formula (I-A):

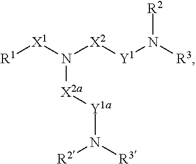

or a pharmaceutically acceptable salt thereof, wherein:
   R¹ is —OH, -Ria

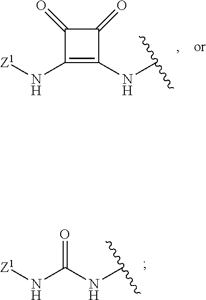

Z¹ is optionally substituted C₁-C₆ alkyl;
X¹ is optionally substituted C₂-C₆ alkylenyl;
X² and X^{2a} are independently optionally substituted C₂-C₁₄ alkylenyl;
Y¹ and Y^{1a} are independently a bond,

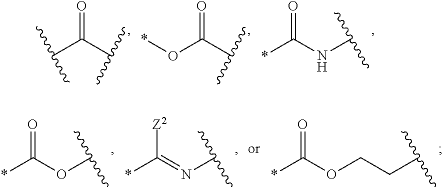

wherein the bond marked with an “*” is attached to X² or X^{2a};
   Z² is H or optionally substituted C₁-C₈ alkyl;
   R² and R³ are independently optionally substituted C₄-C₁₄ alkyl;
   R^{2′} and R^{3′} are independently optionally substituted C₄-C₁₄ alkyl;
   R^{1a} is:

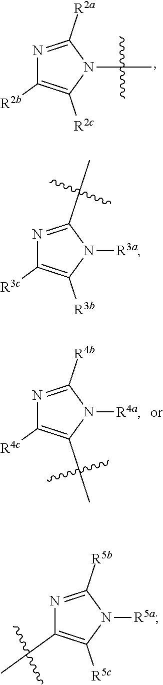

R^{3a}, R^{2b}, and R^{2c} are independently hydrogen or C₁-C₆ alkyl;
R^{3a}, R^{3b}, and R^{3c} are independently hydrogen or C₁-C₆ alkyl;
R^{4a}, R^{4b}, and R^{4c} are independently hydrogen or C₁-C₆ alkyl; and
R^{8a} R^{8b} and R^{1c} are independently hydrogen or C₁-C₆ alkyl.

In some embodiments, Lipids of the Disclosure have a structure of Formula (I-A), wherein R¹ is OH.

In some embodiments, Lipids of the Disclosure have a structure of Formula (I-A), wherein Y¹ and Y^{1a} are independently

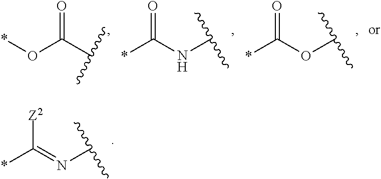

In some embodiments, Lipids of the Disclosure have a structure of Formula (I-A), wherein Y¹ is

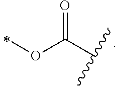

In some embodiments, Lipids of the Disclosure have a structure of Formula (I-A), wherein Y¹ is

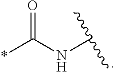 [SMILES: *NC(*)=O]

In some embodiments, Lipids of the Disclosure have a structure of Formula (I-A), wherein Y¹ is

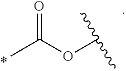 [SMILES: *OC(C)=O]

In some embodiments, Lipids of the Disclosure have a structure of Formula (I-A), wherein Y¹ is

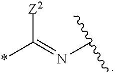

In some embodiments, Lipids of the Disclosure have a structure of Formula (I-A), wherein Y^{1a} is

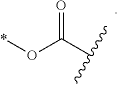 [SMILES: *OC(*)=O]

In some embodiments, Lipids of the Disclosure have a structure of Formula (I-A), wherein Y^{1a} is

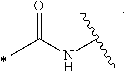 [SMILES: *NC(C)=O]

In some embodiments, Lipids of the Disclosure have a structure of Formula (I-A), wherein Y^{1a} is

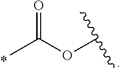

In some embodiments, Lipids of the Disclosure have a structure of Formula (I-A), wherein Y^{1a} is

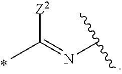

In some embodiments, Lipids of the Disclosure have a structure of Formula (I-A), wherein Z² is H.

In some embodiments, Lipids of the Disclosure have a structure of Formula (I-A), wherein X⁹ is optionally substituted C₂ or C₄ alkylenyl.

In some embodiments, Lipids of the Disclosure have a structure of Formula (I-A), wherein X² and X^{2a} are independently C₄-C₈ alkylenyl (e.g., C₆ alkylenyl). In some embodiments, Lipids of the Disclosure have a structure of Formula (I-A), wherein X² is C₆ alkylenyl. In some embodiments, Lipids of the Disclosure have a structure of Formula (I-A), wherein X^{2a} is C₆ alkylenyl.

In some embodiments, Lipids of the Disclosure have a structure of Formula (I-A), wherein R², R³, R^{2′} and R^{3′} are independently C₄-C₁₄ alkyl (e.g., C₆-C₈ alkyl, C₆, C₇, C₈ alkyl). In some embodiments, Lipids of the Disclosure have a structure of Formula (I-A), wherein R² is C₆-C₈ alkyl. In some embodiments, Lipids of the Disclosure have a structure of Formula (I-A), wherein R³ is C₆-C₈ alkyl. In some embodiments, Lipids of the Disclosure have a structure of Formula (I-A), wherein R² is C₆-C₈ alkyl. In some embodiments, Lipids of the Disclosure have a structure of Formula (I-A), wherein R^{3′} is C₆-C₈ alkyl.

### Formula (II)

In some embodiments, Lipids of the Disclosure have a structure of Formula (II):

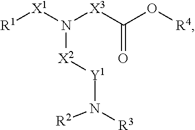

or a pharmaceutically acceptable salt thereof, wherein:
   R¹ is —OH, —R^{1a},

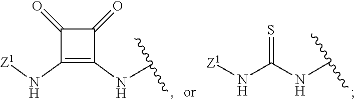

Z¹ is optionally substituted C₁-C₆ alkyl;
X¹ is optionally substituted C₂-C₆ alkylenyl;
X² is optionally substituted C₂-C₁₄ alkylenyl;
Y¹ is a bond,

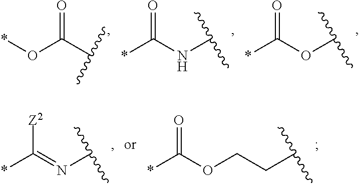

wherein the bond marked with an “*” is attached to X²;
   Z² is H or optionally substituted C₁-C₈ alkyl;
   R² and R³ are independently optionally substituted C₄-C₁₄ alkyl;
   X³ is optionally substituted C₂-C₁₄ alkylenyl;
   R⁴ is optionally substituted C₄-C₁₄ alkyl;
   R^{1a} is:

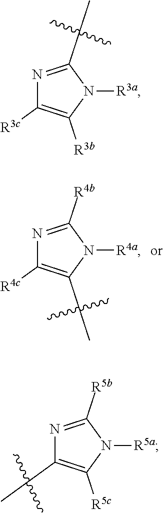

R^{2a}, R^{2b}, and R^{2c} are independently hydrogen or C₁-C₆ alkyl;
R^{3a}, R^{3b}, and R^{3c} are independently hydrogen or C₁-C₆ alkyl;
R^{4a}, R^{4b}, and R^{4c} are independently hydrogen or C₁-C₆ alkyl; and
R^{5a}, R^{5b}, and R^{5c} are independently hydrogen or C₁-C₆ alkyl.

In some embodiments, Lipids of the Disclosure have a structure of Formula (II), wherein R¹ is —OH.

In some embodiments, Lipids of the Disclosure have a structure of Formula (II), wherein X¹ is C₂-C₄ alkylenyl (e.g., C₂ alkylenyl). In some embodiments, Lipids of the Disclosure have a structure of Formula (II), wherein X¹ is C₂ alkylenyl.

In some embodiments, Lipids of the Disclosure have a structure of Formula (II), wherein X² is C₄-C₁₀ alkylenyl (e.g., C₅, C₆, C₇, C₈, C₉ alkyl).

In some embodiments, Lipids of the Disclosure have a structure of Formula (II), wherein Y¹ is

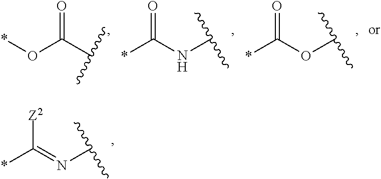

wherein Z² is hydrogen. In some embodiments, Lipids of the Disclosure have a structure of Formula (II), wherein Y¹ is

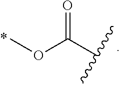 [SMILES: *C(=O)OC]

In some embodiments, Lipids of the Disclosure have a structure of Formula (II), wherein Y¹ is

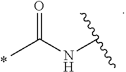 [SMILES: *NC(C)=O]

In some embodiments, Lipids of the Disclosure have a structure of Formula (II), wherein Y¹ is

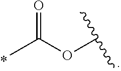

In some embodiments, Lipids of the Disclosure have a structure of Formula (II), wherein Y¹ is

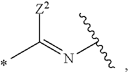

wherein Z² is hydrogen.

In some embodiments, Lipids of the Disclosure have a structure of Formula (II), wherein R² and R³ are independently optionally substituted C₄-C₁₀ alkyl (e.g., C₈ alkyl). In some embodiments, Lipids of the Disclosure have a structure of Formula (II), wherein R² and R³ are independently C₄-C₁₀ alkyl. In some embodiments, Lipids of the Disclosure have a structure of Formula (II), wherein R² and R³ are independently C₈ alkyl.

In some embodiments, Lipids of the Disclosure have a structure of Formula (II), wherein X³ is optionally substituted C₄-C₁₀ alkylenyl (e.g., C₅ alkylenyl). In some embodiments, Lipids of the Disclosure have a structure of Formula (II), wherein X³ is C₄-C₁₀ alkylenyl. In some embodiments, Lipids of the Disclosure have a structure of Formula (II), wherein X³ is C₅ alkylenyl.

In some embodiments, Lipids of the Disclosure have a structure of Formula (II), wherein R⁴ is optionally substituted C₆-C₁₂ alkyl (e.g., C ii alkyl). In some embodiments, Lipids of the Disclosure have a structure of Formula (II), wherein R⁴ is C₆-C₁₂ alkyl. In some embodiments, Lipids of the Disclosure have a structure of Formula (II), wherein R⁴ is C₁₁ alkyl.

### Formula (III-B)

In some embodiments, Lipids of the Disclosure have a structure of Formula (III-B):

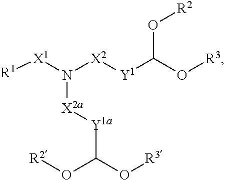

or a pharmaceutically acceptable salt thereof, wherein
   R¹ is

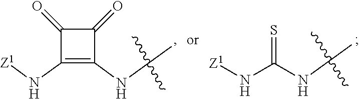

Z¹ is optionally substituted C₁-C₆ alkyl;
X¹ is optionally substituted C₂-C₆ alkylenyl;
X² and X^{2a} are independently optionally substituted C₂-C₁₄ alkylenyl;
Y¹ and Y^{1a} are independently

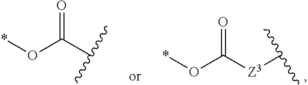

Z³ is independently optionally substituted C₂-C₆ alkylenyl;
R² and R³ are independently optionally substituted C₄-C₁₄ alkyl;
R^{2′} and R^{3′} are independently optionally substituted C₄-C₁₄ alkyl.

In some embodiments, Lipids of the Disclosure have a structure of Formula (III-B), wherein R¹ is

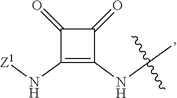

wherein Z¹ is methyl.

In some embodiments, Lipids of the Disclosure have a structure of Formula (II), wherein X¹ is C₂-C₄ alkylenyl (e.g., C₃ alkylenyl). n some embodiments, Lipids of the Disclosure have a structure of Formula (II), wherein X¹ is C₃ alkylenyl.

In some embodiments, Lipids of the Disclosure have a structure of Formula (II), wherein X² is C₄-C₁₀ alkylenyl (e.g., C₆ alkyl). In some embodiments, Lipids of the Disclosure have a structure of Formula (II), wherein X² is C₆ alkyl.

In some embodiments, Lipids of the Disclosure have a structure of Formula (II), wherein R² and R³ are independently optionally substituted C₄-C₁₀ alkyl (e.g., C₈ alkyl). In some embodiments, Lipids of the Disclosure have a structure of Formula (II), wherein R² and R³ are independently C₈ alkyl.

### Formula (III-C)

In some embodiments, Lipids of the Disclosure have a structure of Formula (III-C):

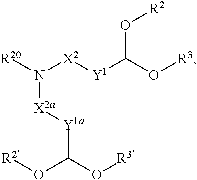

or a pharmaceutically acceptable salt thereof, wherein
   R²⁰ is C₁-C₆ alkylenyl-NR^{20′}C(O)OR^{20″};
   R^{20′}is hydrogen or optionally substituted C₁-C₆ alkyl;
   R^{20″} is optionally substituted C₁-C₆ alkyl, phenyl, or benzyl;
   Z¹ is optionally substituted C₁-C₆ alkyl;
   X² and X^{2a} are independently optionally substituted C₂-C₁₄ alkylenyl;
   Y¹ and Y^{1a} are independently

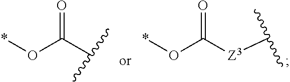

wherein the bond marked with an “*” is attached to X² or X^{2a};
   Z³ is independently optionally substituted C₂-C₆ alkylenyl;
   R² and R³ are independently optionally substituted C₄-C₁₄ alkyl; and
   R^{2′} and R^{3′} are independently optionally substituted C₄-C₁₄ alkyl.

In some embodiments, Lipids of the Disclosure have a structure of Formula (III-C), wherein R²⁰ is —CH₂CH₂CH₂NHC(G)O-t-butyl or —CH₂CH₂CH₂NHC(O)O-benzyl. In some embodiments, Lipids of the Disclosure have a structure of Formula (III-C), wherein R²⁰ is —CH₂CH₂CH₂NHC(O)O-t-butyl. In some embodiments, Lipids of the Disclosure have a structure of Formula (III-C), wherein R²⁰ is —CH₂CH₂CH₂NHC(G)O-benzyl.

In some embodiments, Lipids of the Disclosure have a structure of Formula (III-C), wherein X² and X^{2a} are independently C₄-C₈ alkylenyl (e.g., C₅, C₆, C₇ alkylenyl). In some embodiments, Lipids of the Disclosure have a structure of Formula (III-C), wherein X² is C₆ alkyl. In some embodiments, Lipids of the Disclosure have a structure of Formula (III-C), wherein X^{2a} is C₆ alkyl

In some embodiments, Lipids of the Disclosure have a structure of Formula (III-C), wherein Y¹ and Y^{1a} are

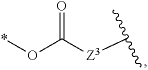

wherein Z³ is C₂-C₄alkylenyl (e.g., C₂ alkylenyl). In some embodiments, Lipids of the Disclosure have a structure of Formula (III-C), wherein Y¹ is

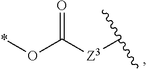

wherein Z³ is C₂-C₄alkylenyl (e.g., C₂ alkylenyl). In some embodiments, Lipids of the Disclosure have a structure of Formula (III-C), wherein Y^{1a} is

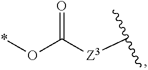

wherein Z³ is C₂-C₄alkylenyl (e.g., C₂ alkylenyl).

In some embodiments, Lipids of the Disclosure have a structure of Formula (III-C), wherein R², R³, R^{2′} and R^{3′} are independently optionally substituted C₄-C₁₀ alkyl (e.g., C₆-C₉alkyl, C₆, C₇, C₈, C₉ alkyl). In some embodiments, Lipids of the Disclosure have a structure of Formula (III-C), wherein R² is C₆-C₉alkyl. In some embodiments, Lipids of the Disclosure have a structure of Formula (III-C), wherein R³ is C₆-C₉alkyl. In some embodiments, Lipids of the Disclosure have a structure of Formula (III-C), wherein R^{2′} is C₆-C₉alkyl. In some embodiments, Lipids of the Disclosure have a structure of Formula (III-C), wherein R^{3′} is C₆-C₉alkyl.

### Formula (III-D)

In some embodiments, Lipids of the Disclosure have a structure of Formula (III-D):

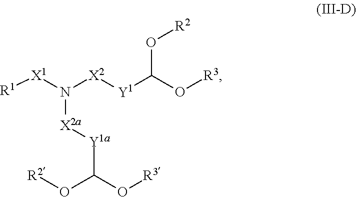

or a pharmaceutically acceptable salt thereof, wherein
R¹ is —OH;
X¹ is optionally substituted C₄ alkylenyl;
X² and X^{2a} are independently optionally substituted C₂-C₁₄ alkylenyl;
Y¹ and Y^{1a} are independently

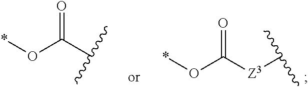

Z³ is independently optionally substituted C₂-C₆ alkylenyl;
   R² and R³ are independently optionally substituted C₄-C₁₄ alkyl or C₁-C₂ alkyl substituted with optionally substituted cyclopropyl; or
R^{2′} and R^{3′} are independently optionally substituted C₄-C₁₄ alkyl or C₁-C₂ alkyl substituted with optionally substituted cyclopropyl.

In some embodiments, Lipids of the Disclosure have a structure of Formula (III-D), wherein X¹ is C₄ alkylenyl.

In some embodiments, Lipids of the Disclosure have a structure of Formula (III-D), wherein X² and X^{2a} are independently optionally substituted C₄-C₁₀ alkylenyl (e.g., C₅, C₆, C₇, C₈, C₉, or C₁₀ alkylenyl). In some embodiments, Lipids of the Disclosure have a structure of Formula (III-D), wherein X² is C₄-C₁₀ alkylenyl. In some embodiments, Lipids of the Disclosure have a structure of Formula (III-D), wherein X^{2a} is C₄-C₁₀ alkylenyl.

In some embodiments, Lipids of the Disclosure have a structure of Formula (III-D), wherein Y¹ and Y^{1a} are independently

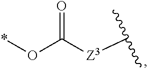

wherein Z³ is independently C₂-C₄ alkylenyl (e.g., C₂, C₄ alkylenyl).

In some embodiments, Lipids of the Disclosure have a structure of Formula (III-D), wherein R², R³, R^{2′} and R^{3′} are independently C₆-C₁₄ alkyl (e.g., C₆, C₇, C₈, C₉, C₁₀, C₁₁, C₁₂, C₁₃, or C₁₄ alkyl) or C₁-C₂ alkyl substituted with optionally substituted cyclopropyl. In some embodiments, Lipids of the Disclosure have a structure of Formula (III-D), wherein R², R³, R^{2′} and R^{3′} are independently C₆-C₁₄ alkyl (e.g., C₆, C₇, C₈, C₉, C₁₀, C₁₁, C₁₂, C₁₃, or C₁₄ alkyl). In some embodiments, Lipids of the Disclosure have a structure of Formula (III-D), wherein R² is C₆-C₁₄ alkyl. In some embodiments, Lipids of the Disclosure have a structure of Formula (III-D), wherein R³ is C₆-C₁₄ alkyl. In some embodiments, Lipids of the Disclosure have a structure of Formula (III-D), wherein R^{2′} is C₆-C₁₄ alkyl. In some embodiments, Lipids of the Disclosure have a structure of Formula (III-D), wherein R³, is C₆-C₁₄ alkyl. In some embodiments, Lipids of the Disclosure have a structure of Formula (III-D), wherein R² is C₁-C₂ alkyl substituted with substituted cyclopropyl. In some embodiments, Lipids of the Disclosure have a structure of Formula (III-D), wherein R³ is C₁-C₂ alkyl substituted with substituted cyclopropyl. In some embodiments, Lipids of the Disclosure have a structure of Formula (III-D), wherein RT is C₁-C₂ alkyl substituted with substituted cyclopropyl. In some embodiments, Lipids of the Disclosure have a structure of Formula (III-D), wherein R^{3′} is C₁-C₂ alkyl substituted with substituted cyclopropyl

In some embodiments, Lipids of the Disclosure have a structure of Formula (III-D), wherein R², R³, R^{2′} and R^{3′} are independently C₁-C₂ alkyl substituted with cyclopropylene-(C₁-C⁶alkylenyl optionally substituted with cyclopropylene substituted with C₁-C₆alkyl). In some embodiments, Lipids of the Disclosure have a structure of Formula (III-D), wherein R² is C₁-C₂ alkyl substituted with cyclopropylene-(C₁-C₆alkylenyl optionally substituted with cyclopropylene substituted with C₁-C₆alkyl). In some embodiments, Lipids of the Disclosure have a structure of Formula (TIT-D), wherein R³ is C₁-C₂ alkyl substituted with cyclopropylene-(C₁-C⁶alkylenyl optionally substituted with cyclopropylene substituted with C₁-C₆alkyl). In some embodiments, Lipids of the Disclosure have a structure of Formula (III-D), wherein R^{2′} is C₁-C₂ alkyl substituted with cyclopropylene-(C₁-C⁶alkylenyl optionally substituted with cyclopropylene substituted with C₁-C₆alkyl). In some embodiments, Lipids of the Disclosure have a structure of Formula (III-D), wherein R^{3′} is C₁-C₂ alkyl substituted with cyclopropylene-(C₁-C⁶alkylenyl optionally substituted with cyclopropylene substituted with C₁-C₆alkyl).

### Formula (III-E)

In some embodiments, Lipids of the Disclosure have a structure of Formula (III-E):

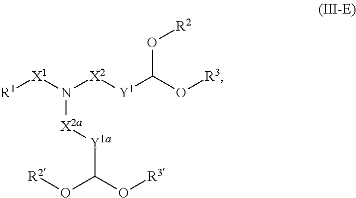

or a pharmaceutically acceptable salt thereof, wherein
   R¹ is —OH;
   X¹ is branched C₂-C₈ alkylenyl
   X² and X^{2a} are independently optionally substituted C₂-C₁₄ alkylenyl;
   Y¹ and Y^{1a} are independently

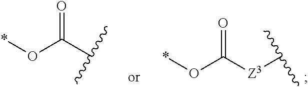

Z is independently optionally substituted C₂-C₆ alkylenyl;
R² and R³ are independently optionally substituted C₄-C₁₄ alkyl;
R^{2′} and R^{3′} are independently optionally substituted C₄-C₁₄ alkyl.

In some embodiments, Lipids of the Disclosure have a structure of Formula (III-E), wherein X¹ is branched C₆ alkylenyl.

In some embodiments, Lipids of the Disclosure have a structure of Formula (III-E), wherein X² and X^{2a} are independently C₄-C₁₀ alkylenyl (e.g., C₆, C₇, C₈ alkylenyl). In some embodiments, Lipids of the Disclosure have a structure of Formula (III-E), wherein X² is C₄-C₁₀ alkylenyl. In some embodiments, Lipids of the Disclosure have a structure of Formula (III-E), wherein X^{2a} is C₄-C₁₀ alkylenyl

In some embodiments, Lipids of the Disclosure have a structure of Formula (III-E), wherein Y¹ and Y^{1a} are

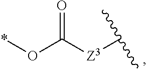

wherein Z³ is independently optionally substituted C₂ alkylenyl. In some embodiments, Lipids of the Disclosure have a structure of Formula (III-E), wherein Y¹ is

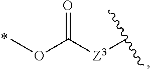

wherein Z³ is independently optionally substituted C₂ alkylenyl. In some embodiments, Lipids of the Disclosure have a structure of Formula (III-E), wherein Y^{1a} is

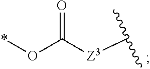

wherein Z³ is independently optionally substituted C₂ alkylenyl.

In some embodiments, Lipids of the Disclosure have a structure of Formula (III-E), wherein R², R³, R^{2′} and R^{3′} are independently C₆-C₁₂ alkyl (e.g., C₉ alkyl) or C₄-C₁₀ alkyl (e.g., C₄, C₆ alkyl) optionally substituted with C₂-C₈alkenylene (e.g., C₄, C₆ alkenylene). In some embodiments, Lipids of the Disclosure have a structure of Formula (III-E), wherein R² is C₆-C₁₂ alkyl. In some embodiments, Lipids of the Disclosure have a structure of Formula (III-E), wherein R³ is C₆-C₁₂ alkyl. In some embodiments, Lipids of the Disclosure have a structure of Formula (III-E), wherein R^{2′} is C₆-C₁₂ alkyl. In some embodiments, Lipids of the Disclosure have a structure of Formula (III-E), wherein R³ is C₆-C₁₂ alkyl. In some embodiments, Lipids of the Disclosure have a structure of Formula (III-E), wherein R² is C₄-C₁₀ alkyl optionally substituted with C₂-C₈alkenylene. In some embodiments, Lipids of the Disclosure have a structure of Formula (III-E), wherein R³ is C₄-C₁₀ alkyl optionally substituted with C₂-C₈alkenylene. In some embodiments, Lipids of the Disclosure have a structure of Formula (III-E), wherein R^{2′} is C₄-C₁₀ alkyl optionally substituted with C₂-C₈alkenylene. In some embodiments, Lipids of the Disclosure have a structure of Formula (III-E), wherein R^{3′} is C₄-C₁₀ alkyl optionally substituted with C₂-C₈alkenylene.

### Formula (III-F)

In some embodiments, Lipids of the Disclosure have a structure of Formula (III-F):

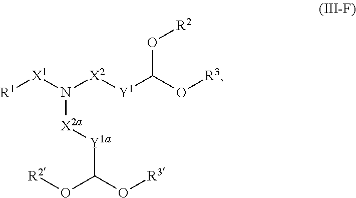

or a pharmaceutically acceptable salt thereof, wherein
R¹ is —OH;
X¹ is optionally substituted C₂-C₆ alkylenyl;
X² and X^{2a} are independently optionally substituted C₂-C₁₄ alkylenyl;
each of Y¹ and Y^{1a} is a bond;
R² and R³ are independently optionally substituted C₄-C₁₄ alkyl; and
R^{2′} and R^{3′} are independently optionally substituted C₄-C₁₄ alkyl.

In some embodiments, Lipids of the Disclosure have a structure of Formula (III-E), wherein X¹ is C₄ alkylenyl.

In some embodiments, Lipids of the Disclosure have a structure of Formula (III-E), wherein X² and X^{2a} are independently C₄-C₁₀ alkylenyl (e.g., C₆-C₈ alkylenyl, C₆, C₇, C₈ alkylenyl). In some embodiments, Lipids of the Disclosure have a structure of Formula (III-E), wherein X² is C₄-C₁₀ alkylenyl. In some embodiments, Lipids of the Disclosure have a structure of Formula (III-E), wherein X^{2a} is C₄-C₁₀ alkylenyl.

In some embodiments, Lipids of the Disclosure have a structure of Formula (III-E), wherein R², R³, R^{2′} and R^{3′} are independently C₆-C₁₀ alkyl (e.g., C₇. C₅ alkyl). In some embodiments, Lipids of the Disclosure have a structure of Formula (III-E), wherein R² is C₆-C₁₀ alkyl. In some embodiments, Lipids of the Disclosure have a structure of Formula (III-E), wherein R³ is C₆-C₁₀ alkyl. In some embodiments, Lipids of the Disclosure have a structure of Formula (III-E), wherein R² is C₆-C₁₀ alkyl. In some embodiments, Lipids of the Disclosure have a structure of Formula (III-E), wherein R^{3′} is C₆-C₁₀ alkyl.

### Formula (VIII-B)

In some embodiments, Lipids of the Disclosure have a structure of Formula (VIII-B):

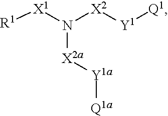

or a pharmaceutically acceptable salt thereof, wherein:
   X¹ is a bond,
   R¹ is C₁-C₆ alkyl,
   X² is C₂-C₆ alkylenyl,
   X^{2a} is C₂-C₁₄ alkylenyl,
   wherein X² or X^{2a} is substituted with OH or C₁₋₄alkylenyl-OH,
   Y¹ is

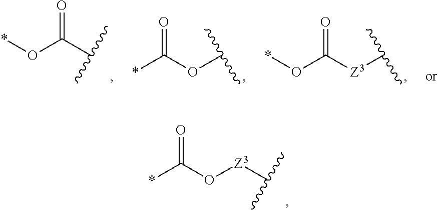

wherein the bond marked with an “*” is attached to X²;
   Y^{1a} is

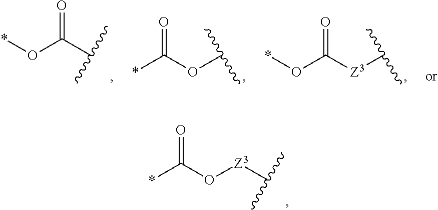

wherein the bond marked with an “*” is attached to X^{2a};
   each Z³ is independently optionally substituted C₁-C₆ alkylenyl or optionally substituted C₂-C₁₄ alkenylenyl;
   Q¹ is —C(R²)(R³)(R¹²);
   Q^{1a} is —C(R^{2′})(R^{3′})(R^{12′});
   R², R³, and R¹² are independently hydrogen, optionally substituted C₁-C₁₄ alkyl, or optionally substituted C₂-C₁₄ alkenylenyl, and
   R^{2′}, R^{3′}, and R^{12′} are independently hydrogen, optionally substituted C₁-C₁₄ alkyl, or optionally substituted C₂-C₁₄ alkenylenyl.

In some embodiments, Lipids of the Disclosure have a structure of Formula (VIII-B), wherein R¹ is methyl.

In some embodiments, Lipids of the Disclosure have a structure of Formula (VIII-B), wherein X² is C₄, C₅, or C₆ alkylenyl.

In some embodiments, Lipids of the Disclosure have a structure of Formula (VIII-B), wherein X^{2a} is C₄-C₅ alkylenyl (e.g., C₅, C₆, or C₇ alkylenyl).

In some embodiments, Lipids of the Disclosure have a structure of Formula (VIII-B), wherein Y¹ is

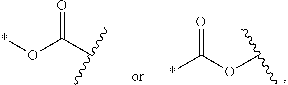 [SMILES: *OC(*)=O ; *OC(*)=O]

### and Y^{1a} is

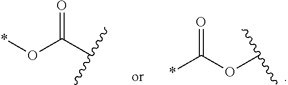 [SMILES: *OC(*)=O ; *OC(*)=O]

In some embodiments, Lipids of the Disclosure have a structure of Formula (VIII-B), wherein Y¹ is

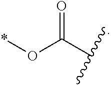 [SMILES: *OC(*)=O]

In some embodiments, Lipids of the Disclosure have a structure of Formula (VIII-B), wherein Y¹ is

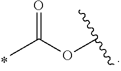 [SMILES: *OC(C)=O]

In some embodiments, Lipids of the Disclosure have a structure of Formula (VIII-B), wherein Y^{1a} is

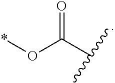 [SMILES: *OC(*)=O]

In some embodiments, Lipids of the Disclosure have a structure of Formula (VIII-B), wherein Y^{1a} is

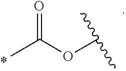 [SMILES: *OC(C)=O]

In some embodiments, Lipids of the Disclosure have a structure of Formula (VIII-B), wherein R², R³, R¹², R^{2′}, R^{3′}, and R^{12′} are independently hydrogen or C₅-C₁₂ alkyl (e.g., C₆, C₇, C₈, C₉, C₁₀, C₁₁ alkyl). In some embodiments, Lipids of the Disclosure have a structure of Formula (VIII-B), wherein R² is hydrogen. In some embodiments, Lipids of the Disclosure have a structure of Formula (VIII-B), wherein R³ is hydrogen. In some embodiments, Lipids of the Disclosure have a structure of Formula (VIII-B), wherein R^{2′} is hydrogen. In some embodiments, Lipids of the Disclosure have a structure of Formula (VIII-B), wherein R^{3′} is hydrogen. In some embodiments, Lipids of the Disclosure have a structure of Formula (VIII-B), wherein R² is C₅-C₁₂ alkyl. In some embodiments, Lipids of the Disclosure have a structure of Formula (VIII-B), wherein R³ is C₅-C₁₂ alkyl. In some embodiments, Lipids of the Disclosure have a structure of Formula (VIII-B), wherein R^{2′} is C₅-C₁₂ alkyl. In some embodiments, Lipids of the Disclosure have a structure of Formula (VIII-B), wherein R^{3′} is C₅-C₁₂ alkyl.

### Formula (IV)

In some embodiments, Lipids of the Disclosure have a structure of Formula (IV):

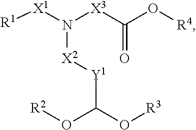

or a pharmaceutically acceptable salt thereof, wherein
R¹ is —OH, —R^{1a},
X¹ is optionally substituted C₂-C₆ alkylenyl;
(i) Y¹ is

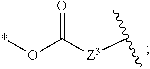

Z³ is optionally substituted C₂-C₆ alkylenyl; and
R² and R³ are independently optionally substituted C₄-C₁₄ alkyl;
X² and X³ are C₅ alkylenyl; or
(ii) Y¹ is a bond
   R² and R³ are independently C₄-C₇alkyl;
   X² is optionally substituted C₂-C₁₄ alkylenyl;
   X³ is optionally substituted C₅ alkylenyl;
R⁴ is optionally substituted C₄-C₁₄ alkyl;
R^{1a} is:

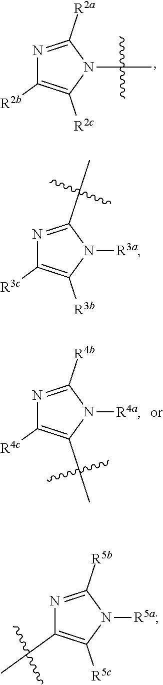

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl.

In some embodiments, Lipids of the Disclosure have a structure of Formula (IV), wherein R¹ is OH.

In some embodiments, Lipids of the Disclosure have a structure of Formula (IV), wherein X¹ is C₂ alkylenyl.

In some embodiments, Lipids of the Disclosure have a structure of Formula (IV), wherein Y¹ is

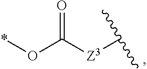

wherein Z³ is C₂ alkylenyl.

In some embodiments, Lipids of the Disclosure have a structure of Formula (IV), wherein R² and R³ are independently C₆-C₁₂ alkyl (C₇, C₈, C₉, C₁₀, C₁₁ alkyl). In some embodiments, Lipids of the Disclosure have a structure of Formula (IV), wherein R² is C₆-C₁₂ alkyl. In some embodiments, Lipids of the Disclosure have a structure of Formula (IV), wherein R³ is C₆-C₁₂ alkyl.

In some embodiments, Lipids of the Disclosure have a structure of Formula (IV), wherein Y¹ is a bond.

In some embodiments, Lipids of the Disclosure have a structure of Formula (IV), wherein R² and R³ are C₄-C₇alkyl (e.g., C₇alkyl). In some embodiments, Lipids of the Disclosure have a structure of Formula (IV), wherein R² is C₄-C₇alkyl. In some embodiments, Lipids of the Disclosure have a structure of Formula (IV), wherein R³ is C₄-C₇alkyl.

In some embodiments, Lipids of the Disclosure have a structure of Formula (IV), wherein X² is C₆-C₁₂ alkylenyl (e.g., C₇, C₈, C₉, C₁₀ alkylenyl).

### Formula (VI)

In some embodiments, Lipids of the Disclosure have a structure of Formula (VI):

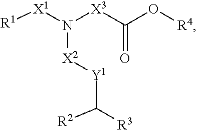

or a pharmaceutically acceptable salt thereof, wherein
   R¹ is —OH,

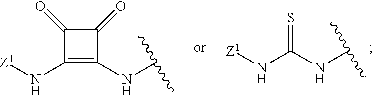

Z¹ is optionally substituted C₁-C₆ alkyl;
X¹ is optionally substituted C₂-C₆ alkylenyl;
X² is optionally substituted C₂-C₁₄ alkylenyl;
X³ is optionally substituted C₂-C₁₄ alkylenyl;
Y¹ is

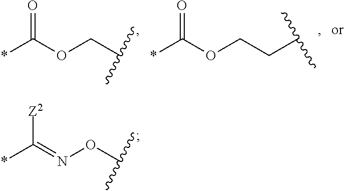

wherein the bond marked with an “*” is attached to X²
   Z² is H or optionally substituted C₁-C₈ alkyl;
   R² and R³ are independently optionally substituted C₃-C₁₄ alkyl; and
   (i) R⁴ is linear C₄-C₁₄ alkyl; or
   (ii) R⁴ is linear C₄-C₁₄ alkyl substituted by 1 or 2 isopropyl groups.

In some embodiments, Lipids of the Disclosure have a structure of Formula (VI), wherein R¹ is —OH.

In some embodiments, Lipids of the Disclosure have a structure of Formula (VI), wherein R¹ is

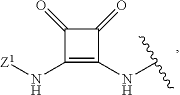

wherein Z¹ is C₁-C₆ alkyl (e.g., methyl).

In some embodiments, Lipids of the Disclosure have a structure of Formula (VI), wherein X¹ is optionally substituted C₂-C₄ alkylenyl (e.g., C₂, C₃, C₄ alkylenyl). In some embodiments, Lipids of the Disclosure have a structure of Formula (VI), wherein X¹ is C₂-C₄ alkylenyl.

In some embodiments, Lipids of the Disclosure have a structure of Formula (VI), wherein X² is C₄-C₈ alkylenyl (e.g., C₅, C₆, C₇, C₈ alkylenyl).

In some embodiments, Lipids of the Disclosure have a structure of Formula (VI), wherein X³ is C₄-C₈ alkylenyl (e.g., C₅, C₆, C₇, C₈ alkylenyl).

In some embodiments, Lipids of the Disclosure have a structure of Formula (VI), wherein Y¹ is

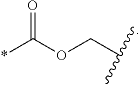 [SMILES: *COC(*)=O]

In some embodiments, Lipids of the Disclosure have a structure of Formula (VI), wherein Y¹ is

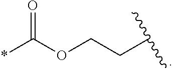 [SMILES: *CCOC(*)=O]

In some embodiments, Lipids of the Disclosure have a structure of Formula (VI), wherein Y¹ is

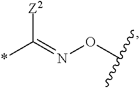

wherein Z² is hydrogen.

In some embodiments, Lipids of the Disclosure have a structure of Formula (VI), wherein R² and R³ are independently C₃-C₈ alkyl (e.g., C₃ alkyl, C₅ alkyl, C₈ alkyl). In some embodiments, Lipids of the Disclosure have a structure of Formula (VI), wherein R² is C₃-C₈ alkyl. In some embodiments, Lipids of the Disclosure have a structure of Formula (VI), wherein R³ is C₃-C₈ alkyl.

In some embodiments, Lipids of the Disclosure have a structure of Formula (VI), wherein R⁴ is linear C₅-C₁₄ alkyl (e.g., C₁o, C₁₁, C₁₂ alkyl).

In some embodiments, Lipids of the Disclosure have a structure of Formula (VI), wherein R⁴ is linear C₄-C₈ alkyl (e.g., C₄alkyl) substituted by 1 or 2 isopropyl groups.

### Formula (X)

In some embodiments, Lipids of the Disclosure have a structure of Formula (X):

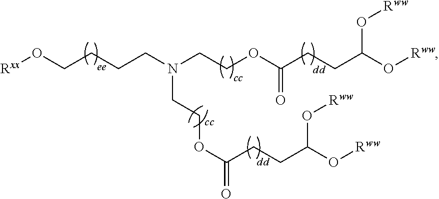

or a pharmaceutically acceptable salt thereof, wherein
each cc is independently selected from 3 to 9;
   R^{xx} is selected from hydrogen and optionally substituted C₁-C₆ alkyl; and
   (i) ee is 1,
      each dd is independently selected from 1 to 4; and
   each R^{ww} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl;
   (ii) ee is 0,
      each dd is 1; and
      each R^{ww} is linear C₄-C₁₂ alkyl.

In some embodiments, Lipids of the Disclosure have a structure of Formula (X), wherein R^{xx} is H. In some embodiments, Lipids of the Disclosure have a structure of Formula (X), wherein R^{xx} is optionally substituted C₁-C₆ alkyl. In some embodiments, Lipids of the Disclosure have a structure of Formula (X), wherein R^{xx} is C₁ alkyl. In some embodiments, Lipids of the Disclosure have a structure of Formula (X), wherein R^{xx} is C₂ alkyl. In some embodiments, Lipids of the Disclosure have a structure of Formula (X), wherein R^{xx} is C₃ alkyl. In some embodiments, Lipids of the Disclosure have a structure of Formula (X), wherein R^{xx} is C₄ alkyl. In some embodiments, Lipids of the Disclosure have a structure of Formula (X), wherein R^{xx} is C₅ alkyl. In some embodiments, Lipids of the Disclosure have a structure of Formula (X), wherein R^{xx} is C₆ alkyl.

In some embodiments, Lipids of the Disclosure have a structure of Formula (X), wherein each R^{ww} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl. In some embodiments, Lipids of the Disclosure have a structure of Formula (X), wherein each R^{ww} is C₄-C₁₄ alkyl, wherein any —(CH₂)₂— of the C₄-C₁₄ alkyl can be optionally replaced with C₂-C₆ cycloalkylenyl. In some embodiments, Lipids of the Disclosure have a structure of Formula (X), wherein each R^{ww} is C₄-C₁₄ alkyl, wherein any —(CH₂)₂— of the C₄-C₁₄ alkyl can be optionally replaced with cyclopropylene. In some embodiments, Lipids of the Disclosure have a structure of Formula (X), wherein each R^{ww} is branched C₄-C₁₂ alkenyl. In some embodiments, Lipids of the Disclosure have a structure of Formula (X), wherein each R^{ww} is C₄-C₁₂ alkenyl comprising at least two double bonds. In some embodiments, Lipids of the Disclosure have a structure of Formula (X), wherein each R^{ww} is C₉-C₁₂ alkenyl. In some embodiments, Lipids of the Disclosure have a structure of Formula (X), wherein each R^{ww} is linear C₄-C₁₂ alkyl. In some embodiments, Lipids of the Disclosure have a structure of Formula (X), wherein each R^{ww} is independently selected from the group consisting of C₆-C₁₄ alkyl, branched C₈-C₁₂ alkenyl, C₈-C₁₂ alkenyl comprising at least two double bonds, and C₉-C₁₂ alkenyl, wherein any —(CH₂)₂— of the C₆-C₁₄ alkyl can be optionally replaced with cyclopropylene. In some embodiments, Lipids of the Disclosure have a structure of Formula (X), wherein each R^{ww} is C₆-C₁₄ alkyl, wherein any —(CH₂)₂— of the C₆-C₁₄ alkyl can be optionally replaced with cyclopropylene. In some embodiments, Lipids of the Disclosure have a structure of Formula (X), wherein each R^{ww} is branched Cs-C₁₂ alkenyl, e.g., (linear or branched C₃-C₈ alkylenyl)-(branched C₅-C₇alkenyl), e.g., (branched C₅ alkylenyl)-(branched C₅alkenyl), e.g.,

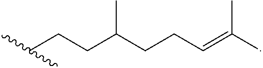

In some embodiments, Lipids of the Disclosure have a structure of Formula (X), wherein each R^{ww} is C₅-C₁₂ alkenyl comprising at least two double bonds. In some embodiments, Lipids of the Disclosure have a structure of Formula (X), wherein each R^{ww} is C₉-C₁₂ alkenyl.

In some embodiments, Lipids of the Disclosure have a structure of Formula (X), wherein each R^{ww} is independently selected from the group consisting of C₆-C₁₄ alkyl (e.g., C₆, C₈, C₉, C₁₀, C₁₁, C₁₃ alkyl), wherein any —(CH₂)₂— of the C₆-C₁₄ alkyl can be optionally replaced with cyclopropylene.

In some embodiments, Lipids of the Disclosure have a structure of Formula (X), wherein each R^{ww} is independently branched C₅-C₁₂ alkenyl (e.g., branched C₁₀ alkenyl).

In some embodiments, Lipids of the Disclosure have a structure of Formula (X), wherein each R^{ww} is independently C₅-C₁₂ alkenyl comprising at least two double bonds (e.g., C₉ or C₁₀ alkenyl comprising two double bonds).

In some embodiments, Lipids of the Disclosure have a structure of Formula (X), wherein each R^{ww} is independently (C₁ alkylenyl)-(cyclopropylene-C₆ alkyl) or (C₂ alkylenyl)-(cyclopropylene-C₂ alkyl). In some embodiments, Lipids of the Disclosure have a structure of Formula (X), wherein each R^{ww} is independently (C₁ alkylenyl)-(cyclopropylene-C₆ alkyl). In some embodiments, Lipids of the Disclosure have a structure of Formula (X), wherein each R^{ww} is independently (C₂ alkylenyl)-(cyclopropylene-C₂ alkyl).

In some embodiments, Lipids of the Disclosure have a structure of Formula (X), wherein each R^{W}W is C₄ alkyl. In some embodiments, Lipids of the Disclosure have a structure of Formula (X), wherein each R^{ww} is C₅ alkyl. In some embodiments, Lipids of the Disclosure have a structure of Formula (X), wherein each R^{ww} is C₆ alkyl. In some embodiments, Lipids of the Disclosure have a structure of Formula (X), wherein each R^{ww} is C₇ alkyl. In some embodiments, Lipids of the Disclosure have a structure of Formula (X), wherein each R^{ww} is C₈ alkyl. In some embodiments, Lipids of the Disclosure have a structure of Formula (X), wherein each R^{ww} is C₉ alkyl. In some embodiments, Lipids of the Disclosure have a structure of Formula (X), wherein each R^{ww} is C₁₀ alkyl. In some embodiments, Lipids of the Disclosure have a structure of Formula (X), wherein each R^{ww} is C₁₁ alkyl. In some embodiments, Lipids of the Disclosure have a structure of Formula (X), wherein each R^{ww} is C₁₂ alkyl. In some embodiments, Lipids of the Disclosure have a structure of Formula (X), wherein each R^{ww} is C₁₃ alkyl. In some embodiments, Lipids of the Disclosure have a structure of Formula (X), wherein each R^{ww} is C₁₄ alkyl.

In some embodiments, Lipids of the Disclosure have a structure of Formula (X), wherein each R^{ww} is C₉ alkenyl. In some embodiments, Lipids of the Disclosure have a structure of Formula (X), wherein each R^{ww} is C₁₀ alkenyl. In some embodiments, Lipids of the Disclosure have a structure of Formula (X), wherein each R^{ww} is C₁₁ alkenyl. In some embodiments, Lipids of the Disclosure have a structure of Formula (X), wherein each R^{ww} is C₁₂ alkenyl.

In some embodiments, Lipids of the Disclosure have a structure of Formula (X), wherein each R^{ww} is C₈ alkenyl comprising at least two double bonds. In some embodiments, Lipids of the Disclosure have a structure of Formula (X), wherein each R^{ww} is C₉ alkenyl comprising at least two double bonds. In some embodiments, Lipids of the Disclosure have a structure of Formula (X), wherein each R^{ww} is C₁₀ alkenyl comprising at least two double bonds. In some embodiments, Lipids of the Disclosure have a structure of Formula (X), wherein each R^{ww} is C₁₁ alkenyl comprising at least two double bonds. In some embodiments, Lipids of the Disclosure have a structure of Formula (X), wherein each R^{ww} is C₁₂ alkenyl comprising at least two double bonds. In some embodiments, Lipids of the Disclosure have a structure of Formula (X), wherein each R^{ww} is C₁₃ alkenyl comprising at least two double bonds. In some embodiments, Lipids of the Disclosure have a structure of Formula (X), wherein each R^{ww} is C₁₄ alkenyl comprising at least two double bonds.

In some embodiments, Lipids of the Disclosure have a structure of Formula (X), wherein each R^{ww} is C₉ alkyl, wherein one —(CH₂)₂— of the C₉ alkyl is replaced with C₂-C₆ cycloalkylenyl. In some embodiments, Lipids of the Disclosure have a structure of Formula (X), wherein each R^{ww} is C₉ alkyl, wherein one —(CH₂)₂— of the C₉ alkyl is replaced with cyclopropylene. In some embodiments, Lipids of the Disclosure have a structure of Formula (X), wherein each R^{ww} is C₉ alkyl, wherein two —(CH₂)₂— of the C₉ alkyl are replaced with C₂-C₆ cycloalkylenyl. In some embodiments, Lipids of the Disclosure have a structure of Formula (X), wherein each R^{ww} is C₉ alkyl, wherein two —(CH₂)₂— of the C₉ alkyl are replaced with cyclopropylene.

In some embodiments, Lipids of the Disclosure have a structure of Formula (X), wherein each R^{ww} is linear C₄ alkyl. In some embodiments, Lipids of the Disclosure have a structure of Formula (X), wherein each R^{ww} is linear C₅ alkyl. In some embodiments, Lipids of the Disclosure have a structure of Formula (X), wherein each R^{ww} is linear C₆ alkyl. In some embodiments, Lipids of the Disclosure have a structure of Formula (X), wherein each R^{ww} is linear C₇ alkyl. In some embodiments, Lipids of the Disclosure have a structure of Formula (X), wherein each R^{ww} is linear C₈ alkyl. In some embodiments, Lipids of the Disclosure have a structure of Formula (X), wherein each R^{ww} is linear C₉ alkyl. In some embodiments, Lipids of the Disclosure have a structure of Formula (X), wherein each R^{ww} is linear C₁₀ alkyl. In some embodiments, Lipids of the Disclosure have a structure of Formula (X), wherein each R^{ww} is linear C₁₁ alkyl. In some embodiments, Lipids of the Disclosure have a structure of Formula (X), wherein each R^{ww} is linear C₁₂ alkyl. In some embodiments, Lipids of the Disclosure have a structure of Formula (X), wherein each R^{ww} is linear C₁₃ alkyl. In some embodiments, Lipids of the Disclosure have a structure of Formula (X), wherein each R^{ww} is linear C₁₄ alkyl.

In some embodiments, Lipids of the Disclosure have a structure of Formula (X), wherein each R^{ww} is branched C₅ alkenyl. In some embodiments, Lipids of the Disclosure have a structure of Formula (X), wherein each R^{ww} is branched C₉ alkenyl. In some embodiments, Lipids of the Disclosure have a structure of Formula (X), wherein each R^{ww} is branched C₁₀ alkenyl. In some embodiments, Lipids of the Disclosure have a structure of Formula (X), wherein each R^{ww} is branched C₁₁ alkenyl. In some embodiments, Lipids of the Disclosure have a structure of Formula (X), wherein each R^{ww} is branched C₂ alkenyl.

In some embodiments, Lipids of the Disclosure have a structure of Formula (X), wherein each cc is independently selected from 3 to 7. In some embodiments, Lipids of the Disclosure have a structure of Formula (X), wherein each cc is 3. In some embodiments, Lipids of the Disclosure have a structure of Formula (X), wherein each cc is 4. In some embodiments, Lipids of the Disclosure have a structure of Formula (X), wherein each cc is 5. In some embodiments, Lipids of the Disclosure have a structure of Formula (X), wherein each cc is 6. In some embodiments, Lipids of the Disclosure have a structure of Formula (X), wherein each cc is 7. In some embodiments, Lipids of the Disclosure have a structure of Formula (X), wherein each cc is 8. In some embodiments, Lipids of the Disclosure have a structure of Formula (X), wherein each cc is 9.

In some embodiments, Lipids of the Disclosure have a structure of Formula (X), wherein each dd is independently selected from 1 to 4. In some embodiments, Lipids of the Disclosure have a structure of Formula (X), wherein each dd is 1. In some embodiments, Lipids of the Disclosure have a structure of Formula (X), wherein each dd is 2. In some embodiments, Lipids of the Disclosure have a structure of Formula (X), wherein each dd is 3.

In some embodiments, Lipids of the Disclosure have a structure of Formula (X), wherein each dd is 4.

In some embodiments, Lipids of the Disclosure have a structure of Formula (X), wherein ee is 1.

In some embodiments, Lipids of the Disclosure have a structure of Formula (X), wherein ee is 0.

### Formula (X-A)

In some embodiments, Lipids of the Disclosure have a structure of Formula (X), wherein the Lipids of the Disclosure have a structure of Formula (X-A):

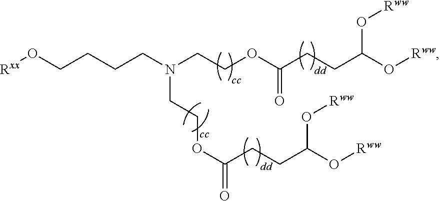

or a pharmaceutically acceptable salt thereof, wherein
each cc is independently selected from 3 to 7;
   each dd is independently selected from 1 to 4;
   R^{xx} is selected from hydrogen and optionally substituted C₁-C₆ alkyl; and
   each R^{ww} is independently selected from the group consisting of C₄-C₁₄ alkyl or (linear or branched C₃-C₈ alkylenyl)-(branched C₅-C₇alkenyl).

In some embodiments, Lipids of the Disclosure have a structure of Formula (X-A), wherein R^{xx} is hydrogen. In some embodiments, Lipids of the Disclosure have a structure of Formula (X-A), wherein R^{xx} is C₁ alkyl. In some embodiments, Lipids of the Disclosure have a structure of Formula (X-A), wherein R^{xx} is C₂ alkyl. In some embodiments, Lipids of the Disclosure have a structure of Formula (X-A), wherein R^{xx} is C₃ alkyl. In some embodiments, Lipids of the Disclosure have a structure of Formula (X-A), wherein R^{xx} is C₄ alkyl. In some embodiments, Lipids of the Disclosure have a structure of Formula (X-A), wherein R^{xx} is C₅ alkyl. In some embodiments, Lipids of the Disclosure have a structure of Formula (X-A), wherein R^{xx} is C₆ alkyl.

In some embodiments, Lipids of the Disclosure have a structure of Formula (X-A), wherein each cc is 4, 5, 6, or 7. In some embodiments, Lipids of the Disclosure have a structure of Formula (X-A), wherein each cc is 3. In some embodiments, Lipids of the Disclosure have a structure of Formula (X-A), wherein each cc is 4. In some embodiments, Lipids of the Disclosure have a structure of Formula (X-A), wherein each cc is 5. In some embodiments, Lipids of the Disclosure have a structure of Formula (X-A), wherein each cc is 6. In some embodiments, Lipids of the Disclosure have a structure of Formula (X-A), wherein each cc is 7.

In some embodiments, Lipids of the Disclosure have a structure of Formula (X-A), wherein each dd is 1 or 3. In some embodiments, Lipids of the Disclosure have a structure of Formula (X-A), wherein each dd is 1. In some embodiments, Lipids of the Disclosure have a structure of Formula (X-A), wherein each dd is 2. In some embodiments, Lipids of the Disclosure have a structure of Formula (X-A), wherein each dd is 3. In some embodiments, Lipids of the Disclosure have a structure of Formula (X-A), wherein each dd is 4.

In some embodiments, Lipids of the Disclosure have a structure of Formula (X-A), wherein each R^{ww} is C₄-C₁₄ alkyl. In some embodiments, Lipids of the Disclosure have a structure of Formula (X-A), wherein each R^{ww} is C₄ alkyl. In some embodiments, Lipids of the Disclosure have a structure of Formula (X-A), wherein each R^{ww} is C₅ alkyl. In some embodiments, Lipids of the Disclosure have a structure of Formula (X-A), wherein each R^{ww} is C₆ alkyl. In some embodiments, Lipids of the Disclosure have a structure of Formula (X-A), wherein each R^{ww} is C₇ alkyl. In some embodiments, Lipids of the Disclosure have a structure of Formula (X-A), wherein each R^{ww} is C₈ alkyl. In some embodiments, Lipids of the Disclosure have a structure of Formula (X-A), wherein each R^{ww} is C₉ alkyl. In some embodiments, Lipids of the Disclosure have a structure of Formula (X-A), wherein each R^{ww} is C₁₀ alkyl. In some embodiments, Lipids of the Disclosure have a structure of Formula (X-A), wherein each R^{ww} is C₁₁ alkyl. In some embodiments, Lipids of the Disclosure have a structure of Formula (X-A), wherein each R^{ww} is C₁₂ alkyl. In some embodiments, Lipids of the Disclosure have a structure of Formula (X-A), wherein each R^{ww} is C₁₃ alkyl. In some embodiments, Lipids of the Disclosure have a structure of Formula (X-A), wherein each R^{1W} is C₁₄ alkyl.

In some embodiments, Lipids of the Disclosure have a structure of Formula (X-A), wherein each R^{ww} is (linear or branched C₃-C₅ alkylenyl)-(branched C₅-C₇alkenyl), e.g., (branched C₅ alkylenyl)-(branched C₅alkenyl), e.g.,

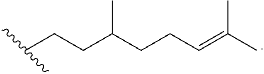 [SMILES: *CCC(C)CCC=C(C)C]

In some embodiments, Lipids of the Disclosure comprise an acyclic core. In some embodiments, Lipids of the Disclosure are selected from any lipid in Table (I) below or a pharmaceutically acceptable salt thereof:

TABLE I
Non-Limiting Examples of Ionizable Lipids with an Acyclic Core

| | Compound |
|---|---|
| Structure | No. |
| 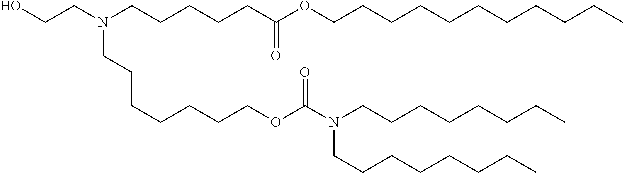 [SMILES: CCCCCCCCCCCOC(=O)CCCCCN(CCO)CCCCCCCOC(=O)N(CCCCCCCC)CCCCCCCC] | 1 |
| 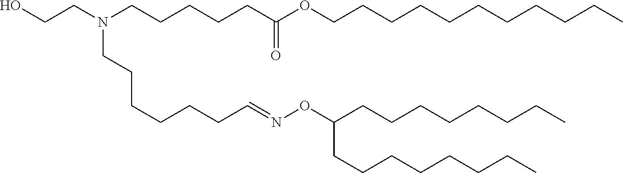 [SMILES: CCCCCCCCCCCOC(=O)CCCCCN(CCO)CCCCCC/C=N/OC(CCCCCCCC)CCCCCCCC] | 2 |
| 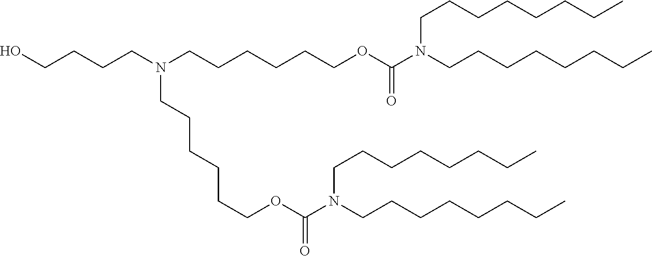 [SMILES: CCCCCCCCN(CCCCCCCC)C(=O)OCCCCCCN(CCCCO)CCCCCCOC(=O)N(CCCCCCCC)CCCCCCCC] | 3 |
| 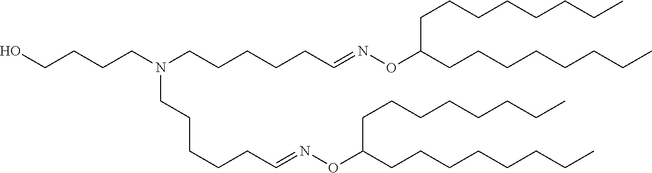 [SMILES: CCCCCCCCC(CCCCCCCC)O/N=C/CCCCCN(CCCCO)CCCCC/C=N/OC(CCCCCCCC)CCCCCCCC] | 4 |
| 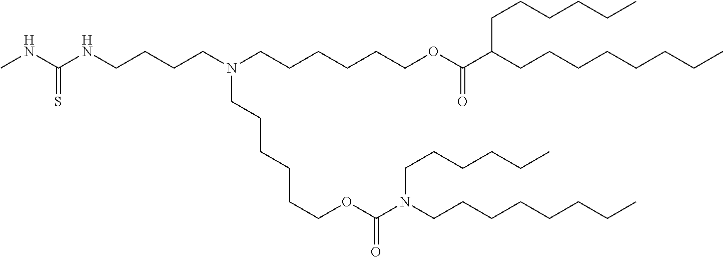 [SMILES: CCCCCCCCC(CCCCCC)C(=O)OCCCCCCN(CCCCCCOC(=O)N(CCCCCC)CCCCCCCC)CCCCNC(=S)NC] | 5 |
| 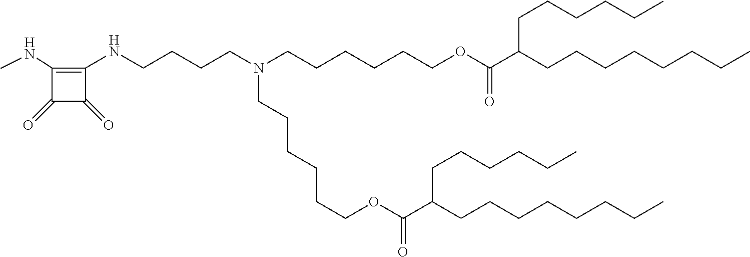 [SMILES: CCCCCCCCC(CCCCCC)C(=O)OCCCCCCN(CCCCCCOC(=O)C(CCCCCC)CCCCCCCC)CCCCNc1c(NC)c(=O)c1=O] | 6 |
| 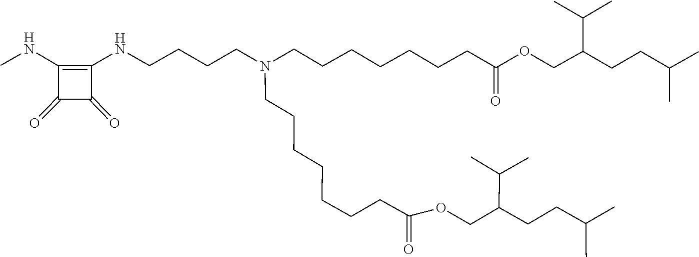 [SMILES: CNc1c(NCCCCN(CCCCCCCC(=O)OCC(CCC(C)C)C(C)C)CCCCCCCC(=O)OCC(CCC(C)C)C(C)C)c(=O)c1=O] | 7 |
| 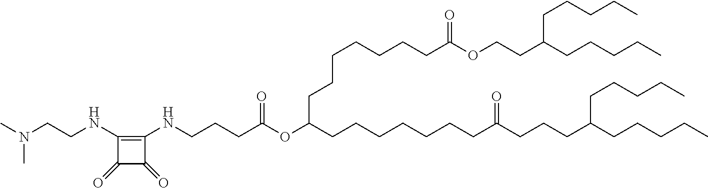 [SMILES: CCCCCC(CCCCC)CCCC(=O)CCCCCCCC(CCCCCCCC(=O)OCCC(CCCCC)CCCCC)OC(=O)CCCNc1c(NCCN(C)C)c(=O)c1=O] | 8 |
| 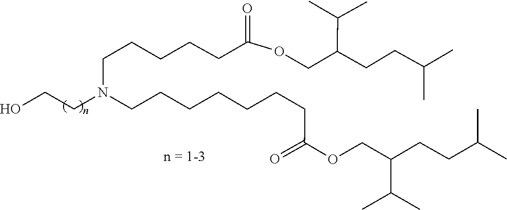 | 9 |
| 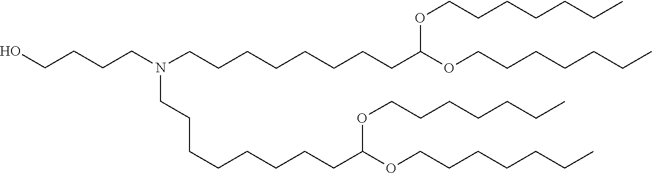 [SMILES: CCCCCCCOC(CCCCCCCCN(CCCCO)CCCCCCCCC(OCCCCCCC)OCCCCCCC)OCCCCCCC] | 10 |
| 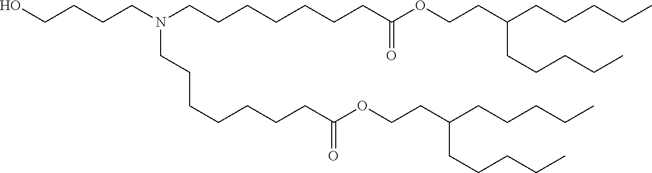 [SMILES: CCCCCC(CCCCC)CCOC(=O)CCCCCCCN(CCCCO)CCCCCCCC(=O)OCCC(CCCCC)CCCCC] | 11 |
| 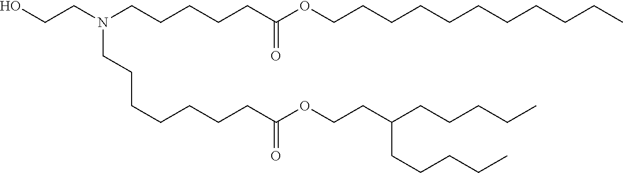 [SMILES: CCCCCCCCCCCOC(=O)CCCCCN(CCO)CCCCCCCC(=O)OCCC(CCCCC)CCCCC] | 13 |
| 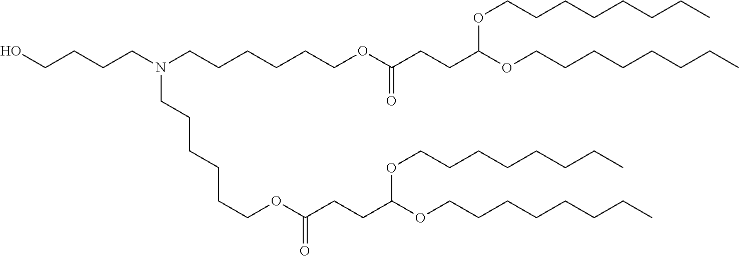 [SMILES: CCCCCCCCOC(CCC(=O)OCCCCCCN(CCCCO)CCCCCCOC(=O)CCC(OCCCCCCCC)OCCCCCCCC)OCCCCCCCC] | 14 |
| 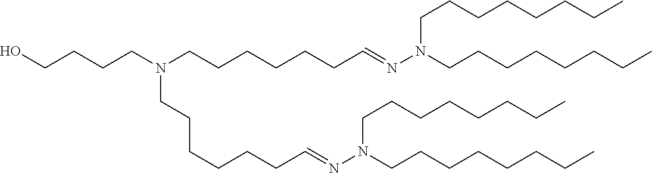 [SMILES: CCCCCCCCN(CCCCCCCC)/N=C/CCCCCCN(CCCCO)CCCCCC/C=N/N(CCCCCCCC)CCCCCCCC] | 15 |
| 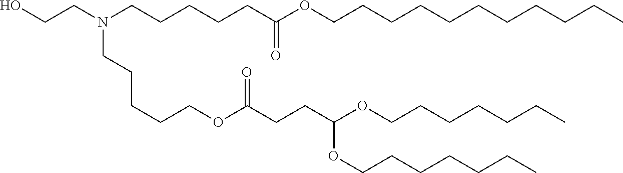 [SMILES: CCCCCCCCCCCOC(=O)CCCCCN(CCO)CCCCCOC(=O)CCC(OCCCCCCC)OCCCCCCC] | 16 |
| 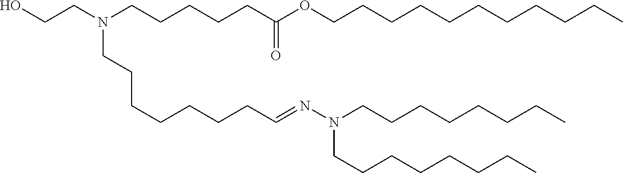 [SMILES: CCCCCCCCCCCOC(=O)CCCCCN(CCO)CCCCCCC/C=N/N(CCCCCCCC)CCCCCCCC] | 17 |
| 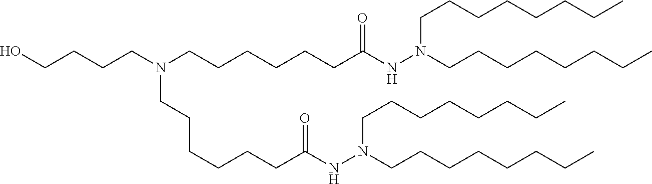 [SMILES: CCCCCCCCN(CCCCCCCC)NC(=O)CCCCCCN(CCCCO)CCCCCCC(=O)NN(CCCCCCCC)CCCCCCCC] | 18 |
| 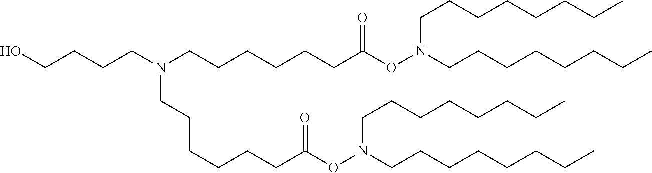 [SMILES: CCCCCCCCN(CCCCCCCC)OC(=O)CCCCCCN(CCCCO)CCCCCCC(=O)ON(CCCCCCCC)CCCCCCCC] | 19 |
| 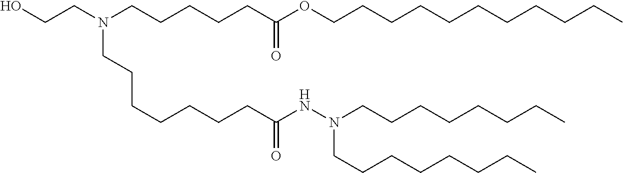 [SMILES: CCCCCCCCCCCOC(=O)CCCCCN(CCO)CCCCCCCC(=O)NN(CCCCCCCC)CCCCCCCC] | 20 |
| 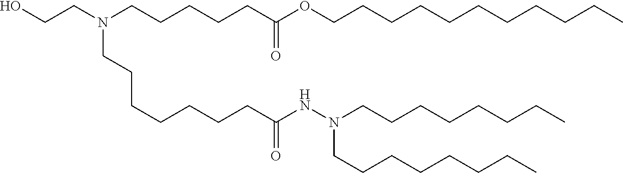 [SMILES: CCCCCCCCCCCOC(=O)CCCCCN(CCO)CCCCCCCC(=O)NN(CCCCCCCC)CCCCCCCC] | 21 |
| 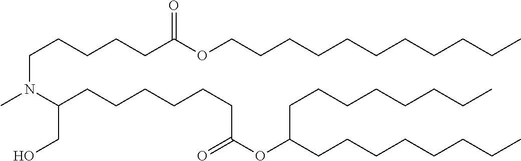 [SMILES: CCCCCCCCCCCOC(=O)CCCCCN(C)C(CO)CCCCCCC(=O)OC(CCCCCCCC)CCCCCCCC] | 22 |
| 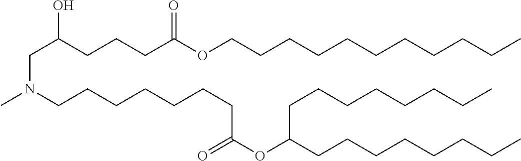 [SMILES: CCCCCCCCCCCOC(=O)CCCC(O)CN(C)CCCCCCCC(=O)OC(CCCCCCCC)CCCCCCCC] | 23 |
| 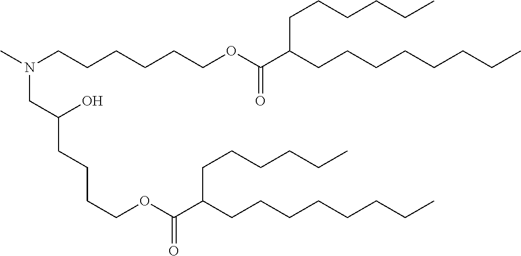 [SMILES: CCCCCCCCC(CCCCCC)C(=O)OCCCCCCN(C)CC(O)CCCCOC(=O)C(CCCCCC)CCCCCCCC] | 24 |
| 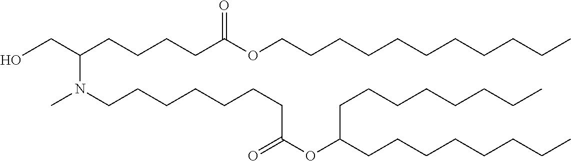 | 25 |
| 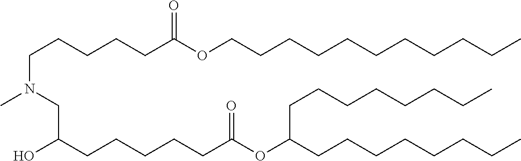 [SMILES: CCCCCCCCCCCOC(=O)CCCCCN(C)CC(O)CCCCCC(=O)OC(CCCCCCCC)CCCCCCCC] | 26 |
| 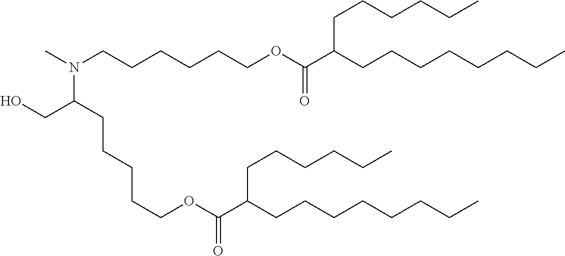 [SMILES: CCCCCCCCC(CCCCCC)C(=O)OCCCCCCN(C)C(CO)CCCCCOC(=O)C(CCCCCC)CCCCCCCC] | 27 |
| 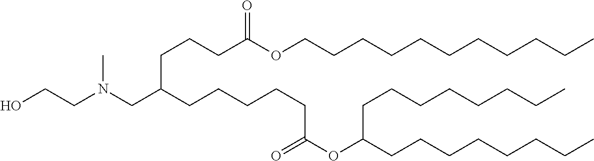 [SMILES: CCCCCCCCCCCOC(=O)CCCC(CCCCCC(=O)OC(CCCCCCCC)CCCCCCCC)CN(C)CCO] | 28 |
| 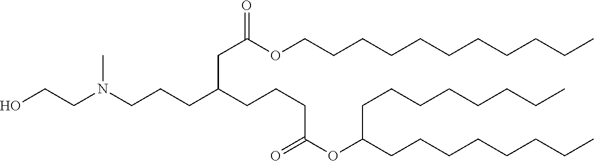 [SMILES: CCCCCCCCCCCOC(=O)CC(CCCC(=O)OC(CCCCCCCC)CCCCCCCC)CCCN(C)CCO] | 29 |
| 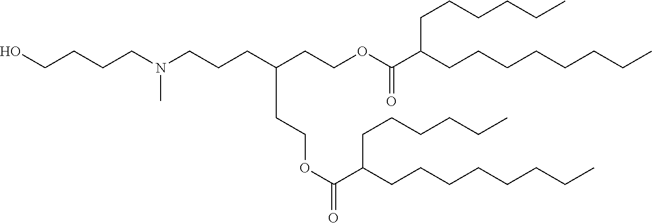 [SMILES: CCCCCCCCC(CCCCCC)C(=O)OCCC(CCCN(C)CCCCO)CCOC(=O)C(CCCCCC)CCCCCCCC] | 30 |
| 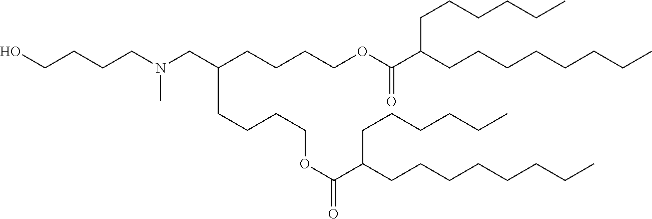 [SMILES: CCCCCCCCC(CCCCCC)C(=O)OCCCCC(CCCCOC(=O)C(CCCCCC)CCCCCCCC)CN(C)CCCCO] | 31 |
| 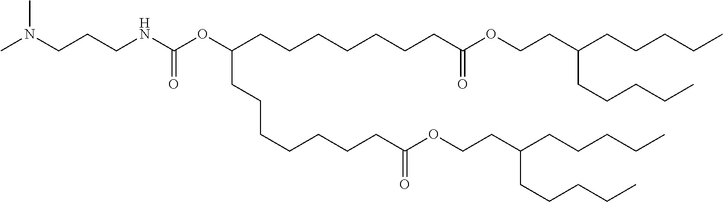 [SMILES: CCCCCC(CCCCC)CCOC(=O)CCCCCCCC(CCCCCCCC(=O)OCCC(CCCCC)CCCCC)OC(=O)NCCCN(C)C] | 32 |
| 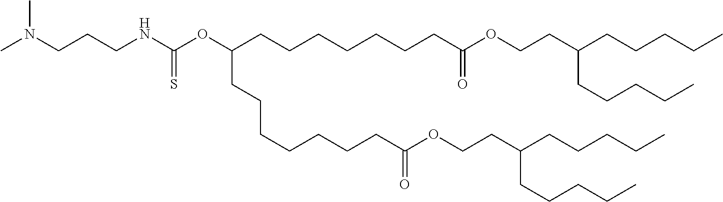 [SMILES: CCCCCC(CCCCC)CCOC(=O)CCCCCCCC(CCCCCCCC(=O)OCCC(CCCCC)CCCCC)OC(=S)NCCCN(C)C] | 33 |
| 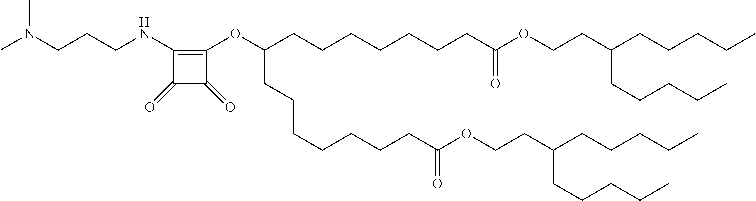 [SMILES: CCCCCC(CCCCC)CCOC(=O)CCCCCCCC(CCCCCCCC(=O)OCCC(CCCCC)CCCCC)Oc1c(NCCCN(C)C)c(=O)c1=O] | 34 |
| 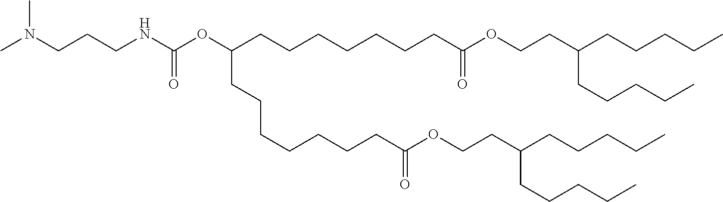 [SMILES: CCCCCC(CCCCC)CCOC(=O)CCCCCCCC(CCCCCCCC(=O)OCCC(CCCCC)CCCCC)OC(=O)NCCCN(C)C] | 35 |
| 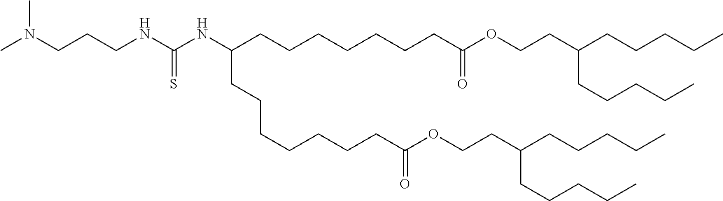 [SMILES: CCCCCC(CCCCC)CCOC(=O)CCCCCCCC(CCCCCCCC(=O)OCCC(CCCCC)CCCCC)NC(=S)NCCCN(C)C] | 36 |
| 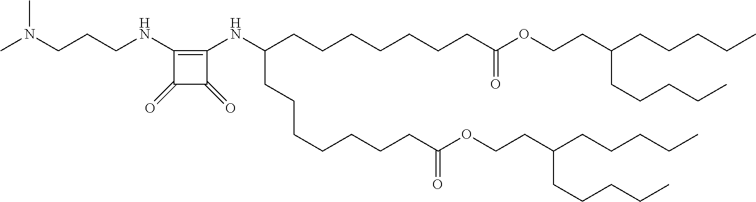 [SMILES: CCCCCC(CCCCC)CCOC(=O)CCCCCCCC(CCCCCCCC(=O)OCCC(CCCCC)CCCCC)Nc1c(NCCCN(C)C)c(=O)c1=O] | 37 |
| 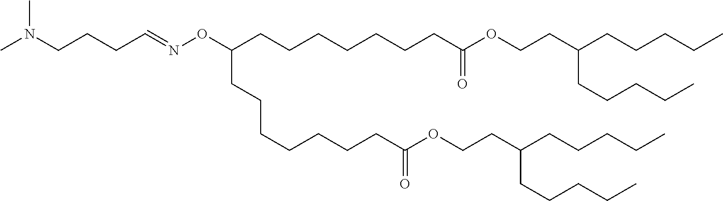 [SMILES: CCCCCC(CCCCC)CCOC(=O)CCCCCCCC(CCCCCCCC(=O)OCCC(CCCCC)CCCCC)O/N=C/CCCN(C)C] | 38 |
| 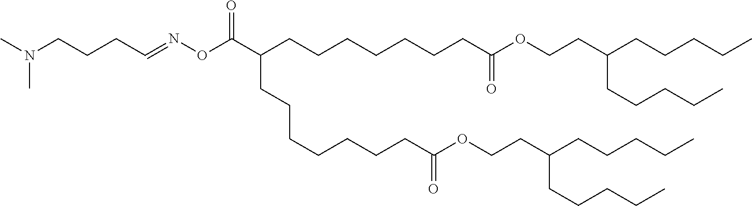 [SMILES: CCCCCC(CCCCC)CCOC(=O)CCCCCCCC(CCCCCCCC(=O)OCCC(CCCCC)CCCCC)C(=O)O/N=C/CCCN(C)C] | 39 |
| 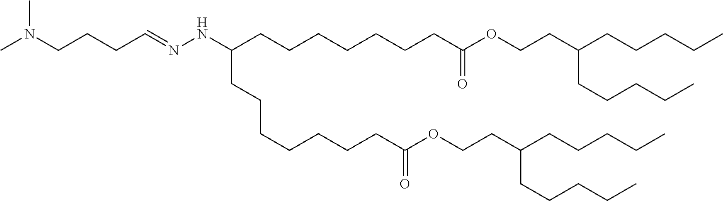 [SMILES: CCCCCC(CCCCC)CCOC(=O)CCCCCCCC(CCCCCCCC(=O)OCCC(CCCCC)CCCCC)N/N=C/CCCN(C)C] | 40 |
| 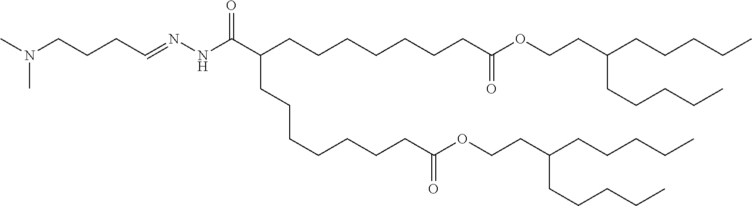 [SMILES: CCCCCC(CCCCC)CCOC(=O)CCCCCCCC(CCCCCCCC(=O)OCCC(CCCCC)CCCCC)C(=O)N/N=C/CCCN(C)C] | 41 |
| 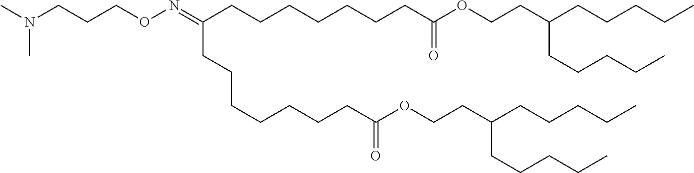 [SMILES: CCCCCC(CCCCC)CCOC(=O)CCCCCCCC(CCCCCCCC(=O)OCCC(CCCCC)CCCCC)=NOCCCN(C)C] | 42 |
| 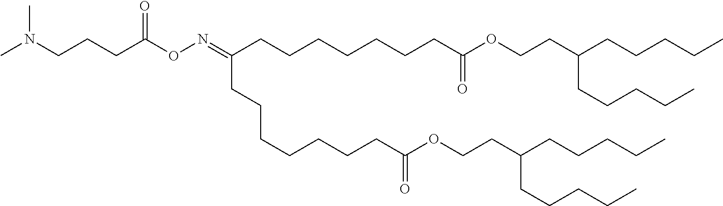 [SMILES: CCCCCC(CCCCC)CCOC(=O)CCCCCCCC(CCCCCCCC(=O)OCCC(CCCCC)CCCCC)=NOC(=O)CCCN(C)C] | 43 |
| 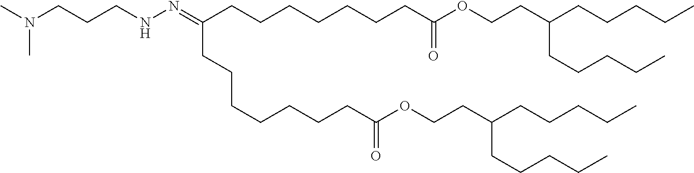 [SMILES: CCCCCC(CCCCC)CCOC(=O)CCCCCCCC(CCCCCCCC(=O)OCCC(CCCCC)CCCCC)=NNCCCN(C)C] | 44 |
| 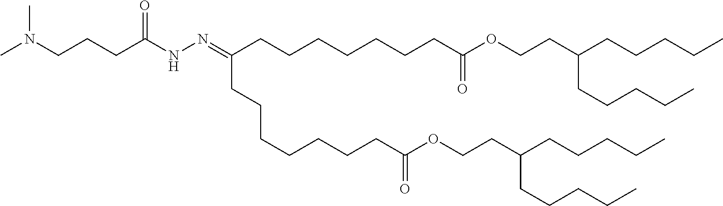 [SMILES: CCCCCC(CCCCC)CCOC(=O)CCCCCCCC(CCCCCCCC(=O)OCCC(CCCCC)CCCCC)=NNC(=O)CCCN(C)C] | 45 |
| 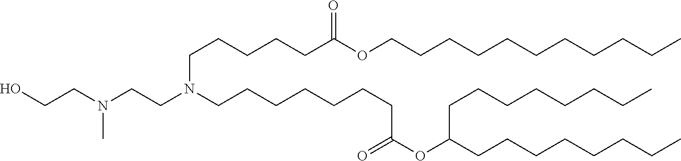 [SMILES: CCCCCCCCCCCOC(=O)CCCCCN(CCCCCCCC(=O)OC(CCCCCCCC)CCCCCCCC)CCN(C)CCO] | 46 |
| 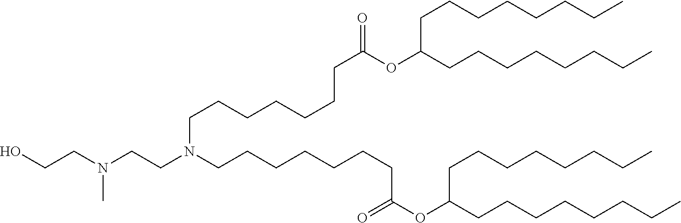 [SMILES: CCCCCCCCC(CCCCCCCC)OC(=O)CCCCCCCN(CCCCCCCC(=O)OC(CCCCCCCC)CCCCCCCC)CCN(C)CCO] | 47 |
| 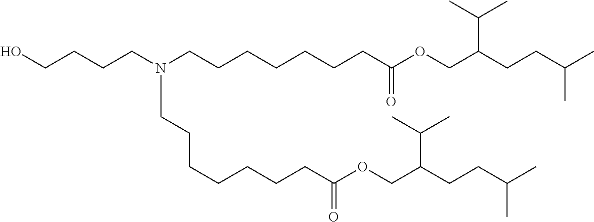 [SMILES: CC(C)CCC(COC(=O)CCCCCCCN(CCCCO)CCCCCCCC(=O)OCC(CCC(C)C)C(C)C)C(C)C] | 48 |
| 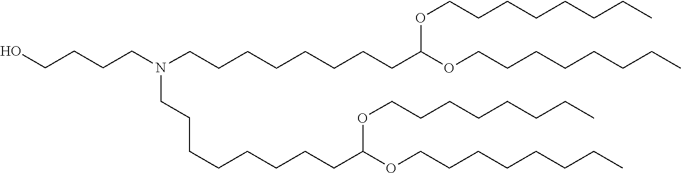 [SMILES: CCCCCCCCOC(CCCCCCCCN(CCCCO)CCCCCCCCC(OCCCCCCCC)OCCCCCCCC)OCCCCCCCC] | 49 |
| 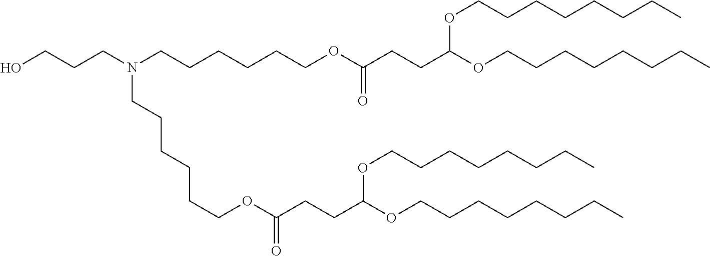 [SMILES: CCCCCCCCOC(CCC(=O)OCCCCCCN(CCCO)CCCCCCOC(=O)CCC(OCCCCCCCC)OCCCCCCCC)OCCCCCCCC] | 50 |
| 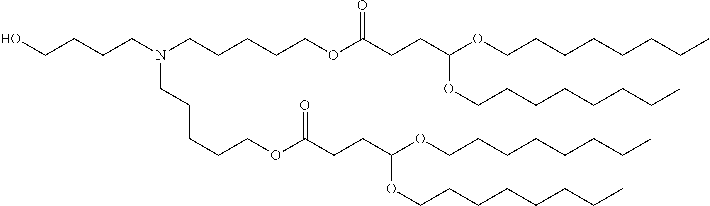 [SMILES: CCCCCCCCOC(CCC(=O)OCCCCCN(CCCCO)CCCCCOC(=O)CCC(OCCCCCCCC)OCCCCCCCC)OCCCCCCCC] | 51 |
| 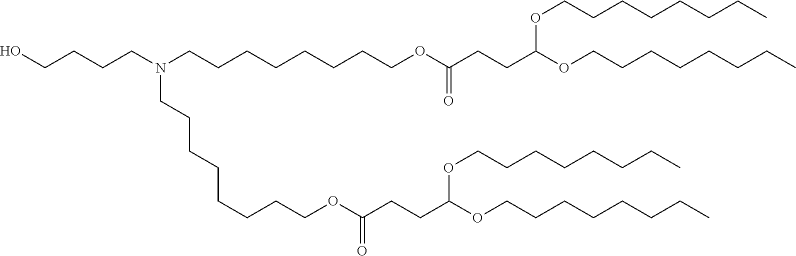 [SMILES: CCCCCCCCOC(CCC(=O)OCCCCCCCCN(CCCCO)CCCCCCCCOC(=O)CCC(OCCCCCCCC)OCCCCCCCC)OCCCCCCCC] | 52 |
| 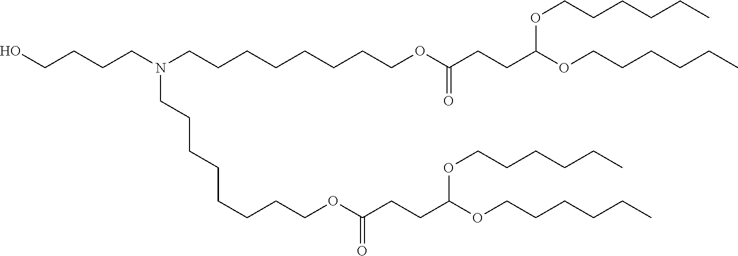 [SMILES: CCCCCCOC(CCC(=O)OCCCCCCCCN(CCCCO)CCCCCCCCOC(=O)CCC(OCCCCCC)OCCCCCC)OCCCCCC] | 53 |
| 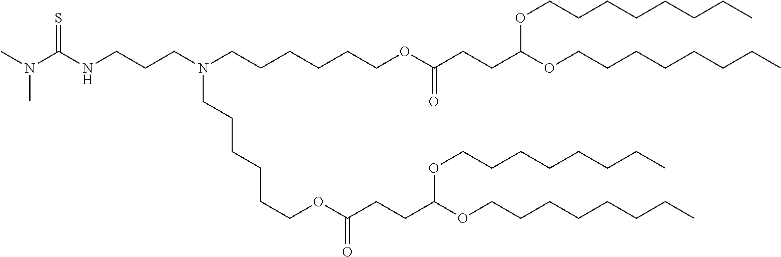 [SMILES: CCCCCCCCOC(CCC(=O)OCCCCCCN(CCCCCCOC(=O)CCC(OCCCCCCCC)OCCCCCCCC)CCCNC(=S)N(C)C)OCCCCCCCC] | 54 |
| 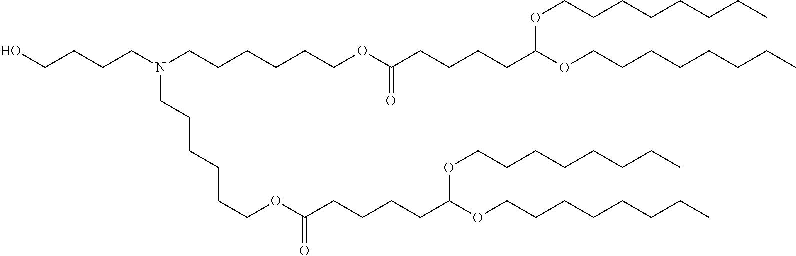 [SMILES: CCCCCCCCOC(CCCCC(=O)OCCCCCCN(CCCCO)CCCCCCOC(=O)CCCCC(OCCCCCCCC)OCCCCCCCC)OCCCCCCCC] | 55 |
| 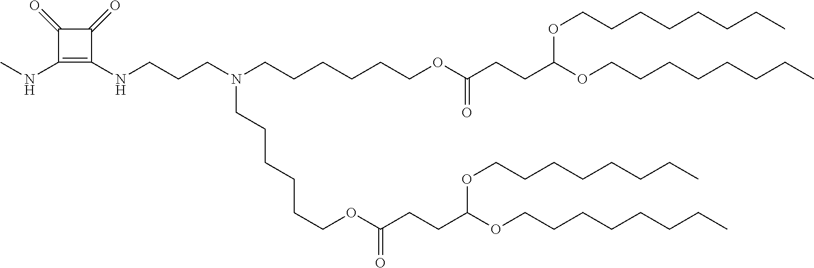 [SMILES: CCCCCCCCOC(CCC(=O)OCCCCCCN(CCCCCCOC(=O)CCC(OCCCCCCCC)OCCCCCCCC)CCCNc1c(NC)c(=O)c1=O)OCCCCCCCC] | 56 |
| 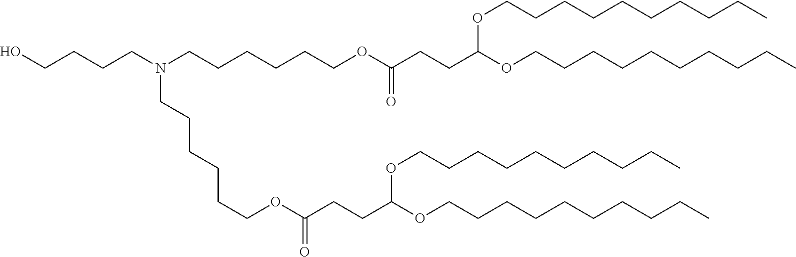 [SMILES: CCCCCCCCCCOC(CCC(=O)OCCCCCCN(CCCCO)CCCCCCOC(=O)CCC(OCCCCCCCCCC)OCCCCCCCCCC)OCCCCCCCCCC] | 57 |
| 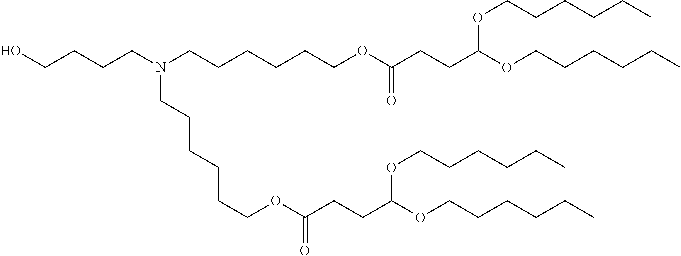 [SMILES: CCCCCCOC(CCC(=O)OCCCCCCN(CCCCO)CCCCCCOC(=O)CCC(OCCCCCC)OCCCCCC)OCCCCCC] | 58 |
| 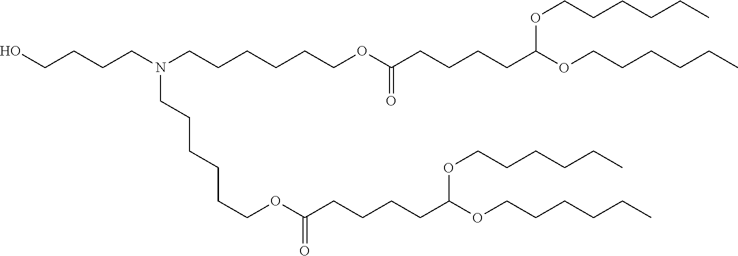 [SMILES: CCCCCCOC(CCCCC(=O)OCCCCCCN(CCCCO)CCCCCCOC(=O)CCCCC(OCCCCCC)OCCCCCC)OCCCCCC] | 59 |
| 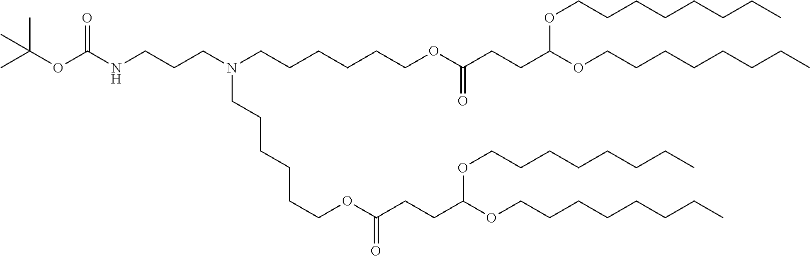 [SMILES: CCCCCCCCOC(CCC(=O)OCCCCCCN(CCCCCCOC(=O)CCC(OCCCCCCCC)OCCCCCCCC)CCCNC(=O)OC(C)(C)C)OCCCCCCCC] | 60 |
| 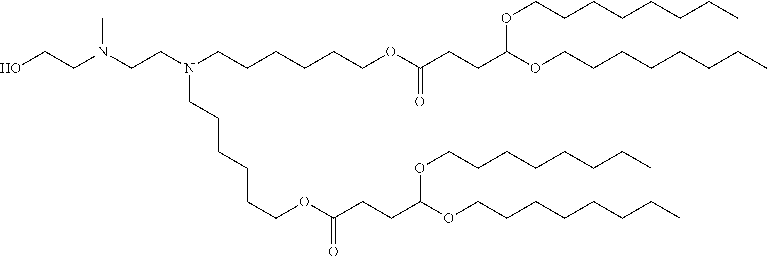 [SMILES: CCCCCCCCOC(CCC(=O)OCCCCCCN(CCCCCCOC(=O)CCC(OCCCCCCCC)OCCCCCCCC)CCN(C)CCO)OCCCCCCCC] | 61 |
| 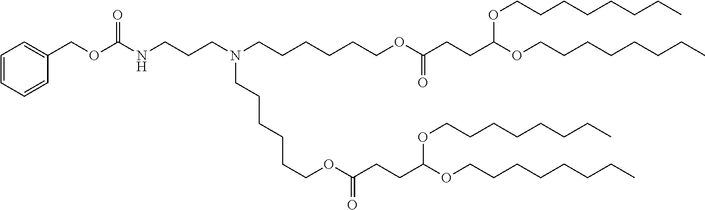 [SMILES: CCCCCCCCOC(CCC(=O)OCCCCCCN(CCCCCCOC(=O)CCC(OCCCCCCCC)OCCCCCCCC)CCCNC(=O)OCc1ccccc1)OCCCCCCCC] | 62 |
| 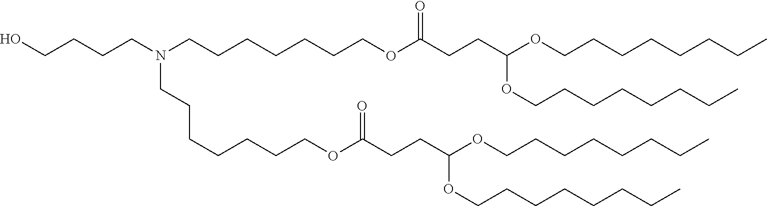 [SMILES: CCCCCCCCOC(CCC(=O)OCCCCCCCN(CCCCO)CCCCCCCOC(=O)CCC(OCCCCCCCC)OCCCCCCCC)OCCCCCCCC] | 64 |
| 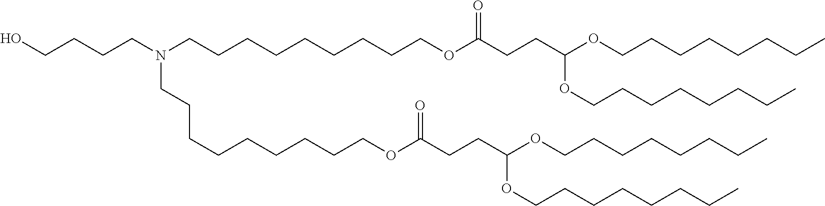 [SMILES: CCCCCCCCOC(CCC(=O)OCCCCCCCCCN(CCCCO)CCCCCCCCCOC(=O)CCC(OCCCCCCCC)OCCCCCCCC)OCCCCCCCC] | 65 |
| 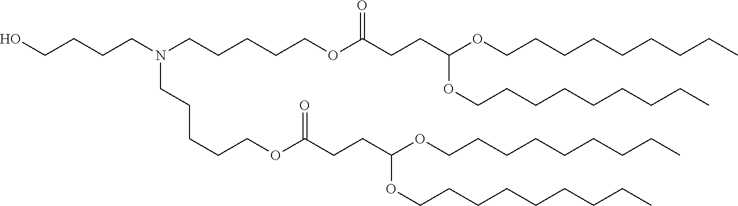 [SMILES: CCCCCCCCCOC(CCC(=O)OCCCCCN(CCCCO)CCCCCOC(=O)CCC(OCCCCCCCCC)OCCCCCCCCC)OCCCCCCCCC] | 66 |
| 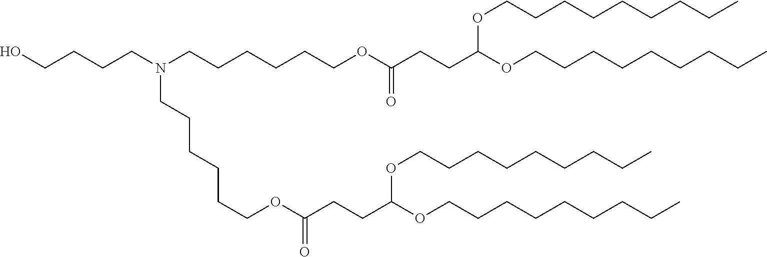 [SMILES: CCCCCCCCCOC(CCC(=O)OCCCCCCN(CCCCO)CCCCCCOC(=O)CCC(OCCCCCCCCC)OCCCCCCCCC)OCCCCCCCCC] | 67 |
| 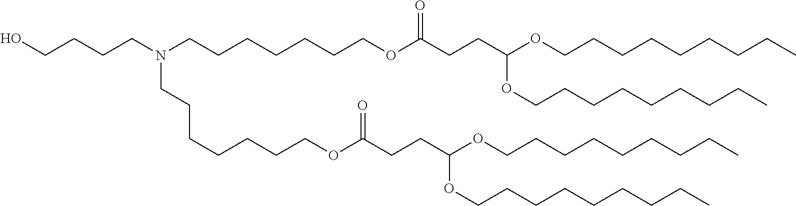 [SMILES: CCCCCCCCCOC(CCC(=O)OCCCCCCCN(CCCCO)CCCCCCCOC(=O)CCC(OCCCCCCCCC)OCCCCCCCCC)OCCCCCCCCC] | 68 |
| 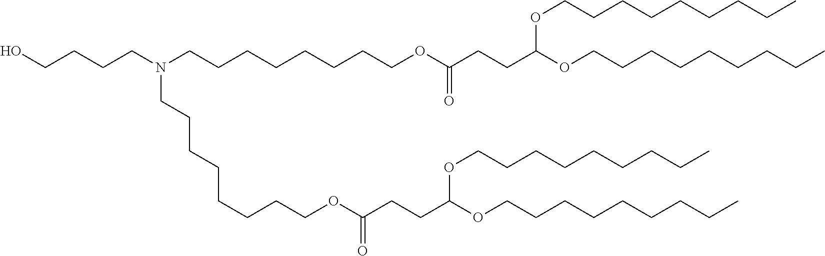 [SMILES: CCCCCCCCCOC(CCC(=O)OCCCCCCCCN(CCCCO)CCCCCCCCOC(=O)CCC(OCCCCCCCCC)OCCCCCCCCC)OCCCCCCCCC] | 69 |
| 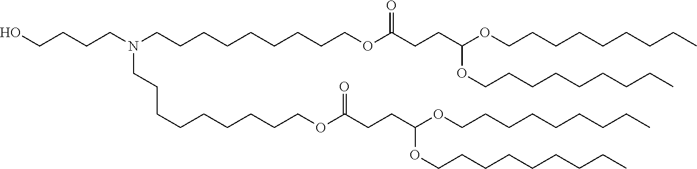 [SMILES: CCCCCCCCCOC(CCC(=O)OCCCCCCCCCN(CCCCO)CCCCCCCCCOC(=O)CCC(OCCCCCCCCC)OCCCCCCCCC)OCCCCCCCCC] | 70 |
| 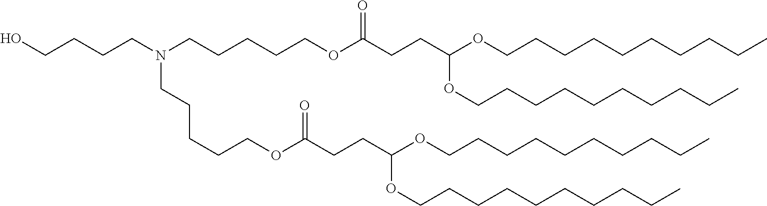 [SMILES: CCCCCCCCCCOC(CCC(=O)OCCCCCN(CCCCO)CCCCCOC(=O)CCC(OCCCCCCCCCC)OCCCCCCCCCC)OCCCCCCCCCC] | 71 |
| 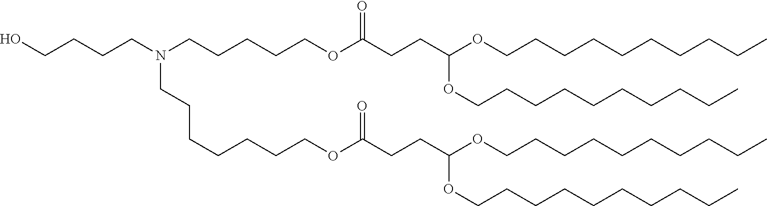 [SMILES: CCCCCCCCCCOC(CCC(=O)OCCCCCCCN(CCCCO)CCCCCOC(=O)CCC(OCCCCCCCCCC)OCCCCCCCCCC)OCCCCCCCCCC] | 72 |
| 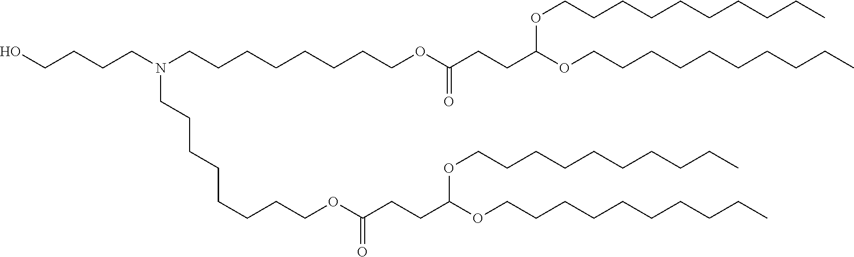 [SMILES: CCCCCCCCCCOC(CCC(=O)OCCCCCCCCN(CCCCO)CCCCCCCCOC(=O)CCC(OCCCCCCCCCC)OCCCCCCCCCC)OCCCCCCCCCC] | 73 |
| 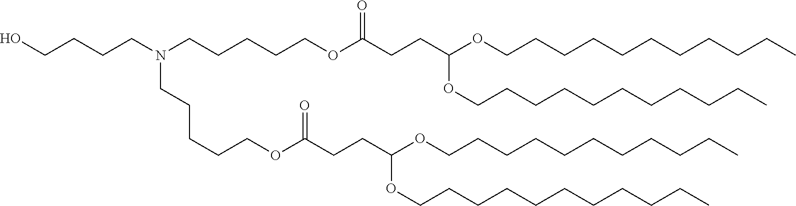 [SMILES: CCCCCCCCCCCOC(CCC(=O)OCCCCCN(CCCCO)CCCCCOC(=O)CCC(OCCCCCCCCCCC)OCCCCCCCCCCC)OCCCCCCCCCCC] | 74 |
| 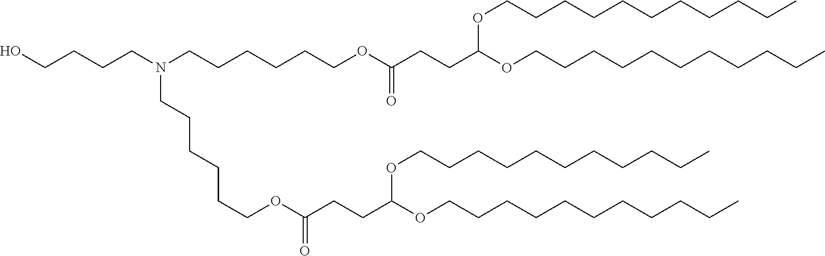 [SMILES: CCCCCCCCCCCOC(CCC(=O)OCCCCCCN(CCCCO)CCCCCCOC(=O)CCC(OCCCCCCCCCCC)OCCCCCCCCCCC)OCCCCCCCCCCC] | 75 |
| 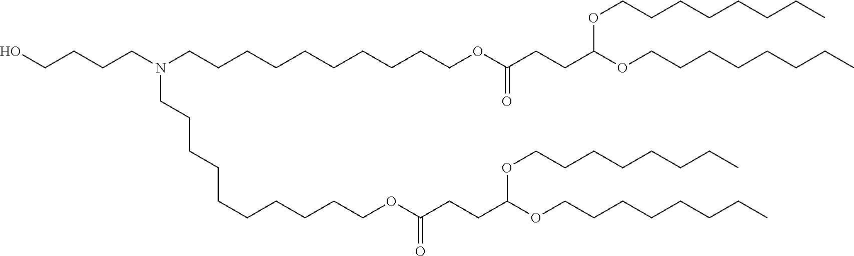 [SMILES: CCCCCCCCOC(CCC(=O)OCCCCCCCCCCN(CCCCO)CCCCCCCCCCOC(=O)CCC(OCCCCCCCC)OCCCCCCCC)OCCCCCCCC] | 76 |
| 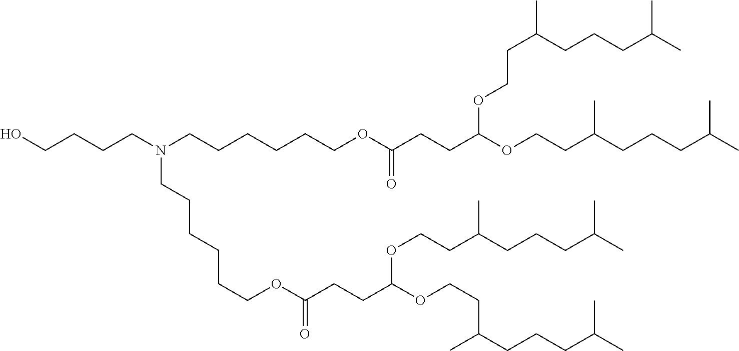 [SMILES: CC(C)CCCC(C)CCOC(CCC(=O)OCCCCCCN(CCCCO)CCCCCCOC(=O)CCC(OCCC(C)CCCC(C)C)OCCC(C)CCCC(C)C)OCCC(C)CCCC(C)C] | 77 |
| 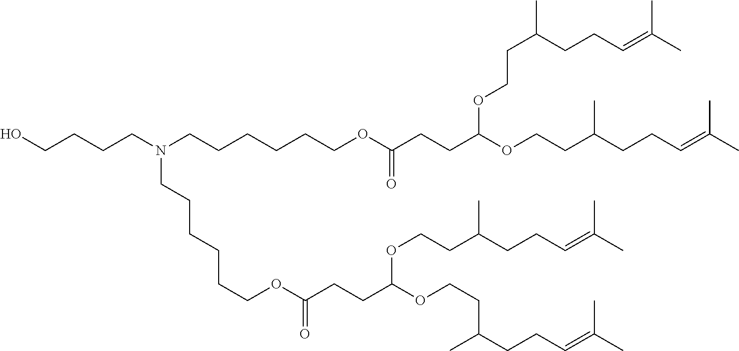 [SMILES: CC(C)=CCCC(C)CCOC(CCC(=O)OCCCCCCN(CCCCO)CCCCCCOC(=O)CCC(OCCC(C)CCC=C(C)C)OCCC(C)CCC=C(C)C)OCCC(C)CCC=C(C)C] | 78 |
| 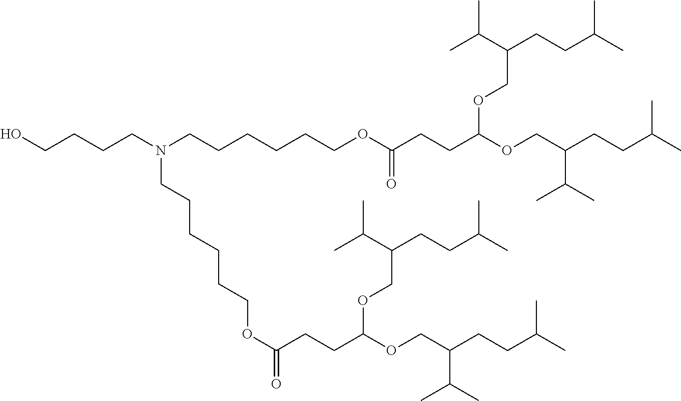 [SMILES: CC(C)CCC(COC(CCC(=O)OCCCCCCN(CCCCO)CCCCCCOC(=O)CCC(OCC(CCC(C)C)C(C)C)OCC(CCC(C)C)C(C)C)OCC(CCC(C)C)C(C)C)C(C)C] | 79 |
| 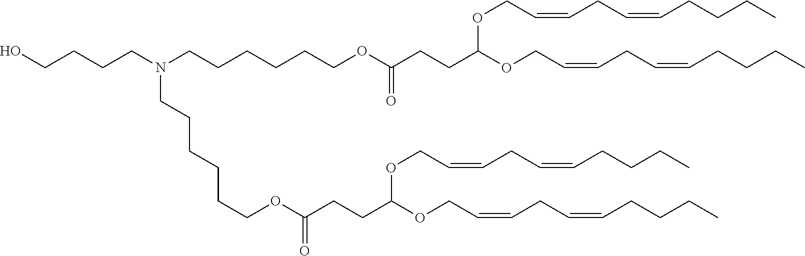 [SMILES: CCCC/C=C\C/C=C\COC(CCC(=O)OCCCCCCN(CCCCO)CCCCCCOC(=O)CCC(OC/C=C\C/C=C\CCCC)OC/C=C\C/C=C\CCCC)OC/C=C\C/C=C\CCCC] | 80 |
| 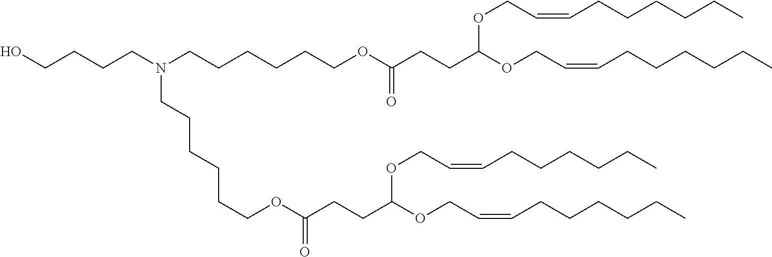 [SMILES: CCCCCC/C=C\COC(CCC(=O)OCCCCCCN(CCCCO)CCCCCCOC(=O)CCC(OC/C=C\CCCCCC)OC/C=C\CCCCCC)OC/C=C\CCCCCC] | 81 |
| 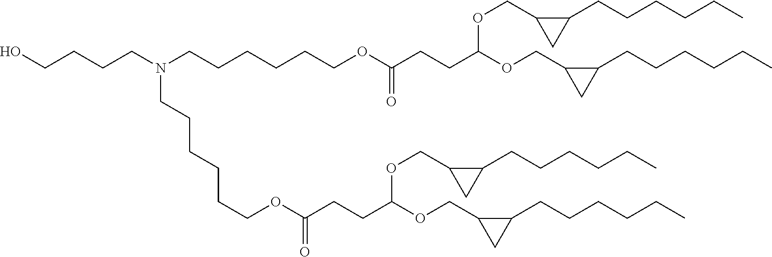 [SMILES: CCCCCCC1CC1COC(CCC(=O)OCCCCCCN(CCCCO)CCCCCCOC(=O)CCC(OCC1CC1CCCCCC)OCC1CC1CCCCCC)OCC1CC1CCCCCC] | 82 |
| 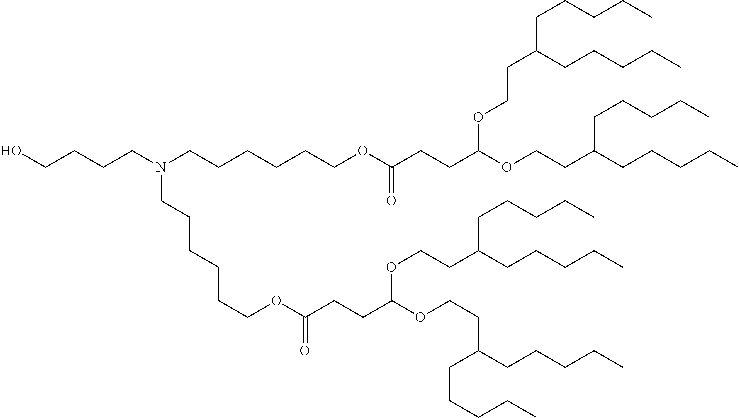 [SMILES: CCCCCC(CCCCC)CCOC(CCC(=O)OCCCCCCN(CCCCO)CCCCCCOC(=O)CCC(OCCC(CCCCC)CCCCC)OCCC(CCCCC)CCCCC)OCCC(CCCCC)CCCCC] | 83 |
| 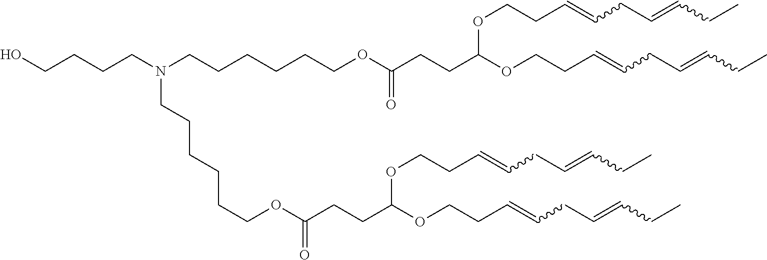 [SMILES: CCC=CCC=CCCOC(CCC(=O)OCCCCCCN(CCCCO)CCCCCCOC(=O)CCC(OCCC=CCC=CCC)OCCC=CCC=CCC)OCCC=CCC=CCC] | 84 |
| 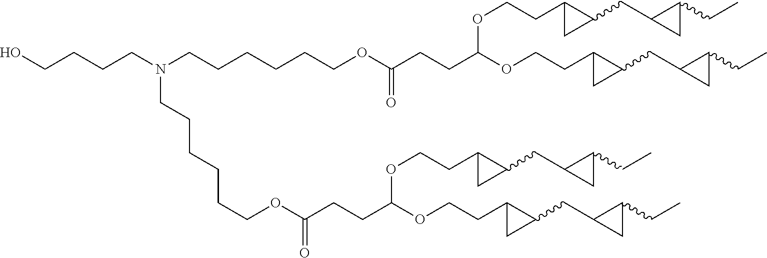 [SMILES: CCC1CC1CC1CC1CCOC(CCC(=O)OCCCCCCN(CCCCO)CCCCCCOC(=O)CCC(OCCC1CC1CC1CC1CC)OCCC1CC1CC1CC1CC)OCCC1CC1CC1CC1CC] | 85 |
| 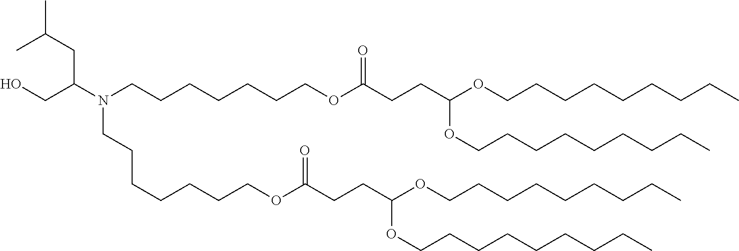 [SMILES: CCCCCCCCCOC(CCC(=O)OCCCCCCCN(CCCCCCCOC(=O)CCC(OCCCCCCCCC)OCCCCCCCCC)C(CO)CC(C)C)OCCCCCCCCC] | 86 |
| 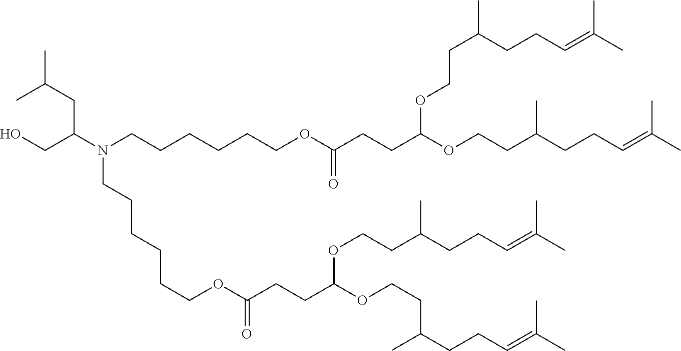 [SMILES: CC(C)=CCCC(C)CCOC(CCC(=O)OCCCCCCN(CCCCCCOC(=O)CCC(OCCC(C)CCC=C(C)C)OCCC(C)CCC=C(C)C)C(CO)CC(C)C)OCCC(C)CCC=C(C)C] | 87 |
| 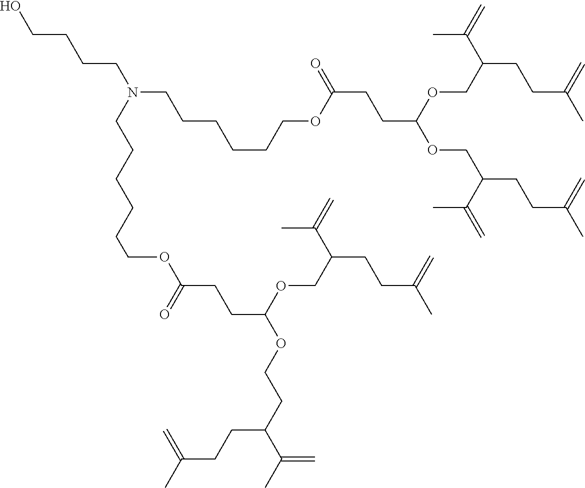 [SMILES: C=C(C)CCC(CCOC(CCC(=O)OCCCCCCN(CCCCO)CCCCCCOC(=O)CCC(OCC(CCC(=C)C)C(=C)C)OCC(CCC(=C)C)C(=C)C)OCC(CCC(=C)C)C(=C)C)C(=C)C] | 88 |
| 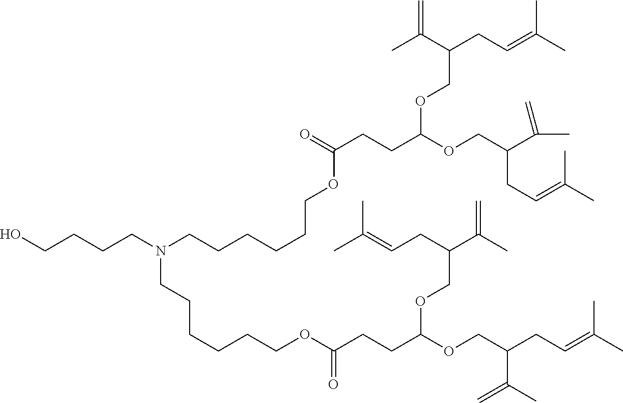 [SMILES: C=C(C)C(CC=C(C)C)COC(CCC(=O)OCCCCCCN(CCCCO)CCCCCCOC(=O)CCC(OCC(CC=C(C)C)C(=C)C)OCC(CC=C(C)C)C(=C)C)OCC(CC=C(C)C)C(=C)C] | 89 |

In some embodiments, an LNP of the present disclosure comprises an ionizable lipid disclosed in PCT Application PCT/US2022/076415.

### Formula (CY)

In some embodiments, an LNP disclosed herein comprises an ionizable lipid of Formula (CY)

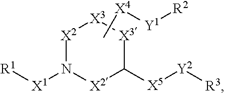

or a pharmaceutically acceptable salt thereof,
wherein:
R¹ is selected from the group consisting of —OH, —OAc, R^{1a},

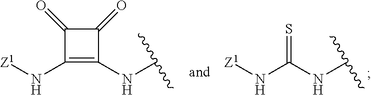

Z¹ is optionally substituted C₁-C₆ alkyl;
X is optionally substituted C₂-C₆ alkylenyl;
X² is selected from the group consisting of a bond, —CH₂— and —CH₂—CH₂;
X^{2′} is selected from the group consisting of a bond, —CH₂— and —CH₂CH₂—;
X³ is selected from the group consisting of a bond, —CH₂— and —CH₂CH₂—;
X^{3′} is selected from the group consisting of a bond, —CH₂— and —CH₂CH₂—;
X⁴ and X⁵ are independently optionally substituted C₂-C₁₄ alkylenyl or optionally substituted C₂-C₁₄ alkenylenyl;
Y¹ and Y² are independently selected from the group consisting of

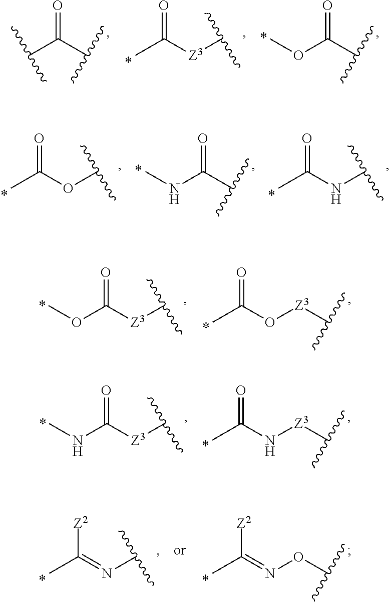

wherein the bond marked with an “*” is attached to X⁴ or X⁵;
each Z² is independently 1-P or optionally substituted C₁-C₈ alkyl;
each Z³ is independently optionally substituted C₁-C₆ alkylenyl;
R² is selected from the group consisting of optionally substituted C₄-C₂₀ alkyl, optionally substituted C₂-C₁₄ alkenyl, and —(CH₂)ₚCH(OR⁶)(OR⁷);
R³ is selected from the group consisting of optionally substituted C₄-C₂₀ alkyl, optionally substituted C₂-C₁₄ alkenyl, or —(CH₂)_{q}CH(OR⁸)(OR⁹);
R^{1a} is:

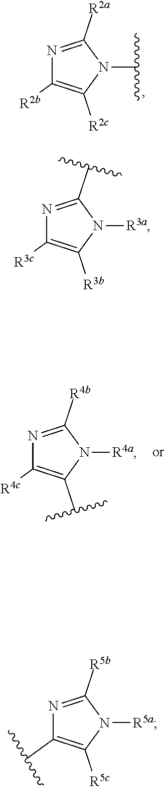

R^{2a}, R^{2b} and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl;
   R^{5a}, R^{5b}, and R^{5c} are independently hydrogen and C₁-C₆ alkyl;
R⁶, R⁷, R⁸, and R⁹ are independently optionally substituted C₁-C₁ alkyl, optionally substituted C₂-C₁₄ alkenyl, or —(CH)ₘ-A-(CH₂)ₙH;
each A is independently a C₈-C₈ cycloalkylenyl;
each n is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
p is selected from the group consisting of 0, 1, 2, 3, 4, 5, 6, and 7; and
q is selected from the group consisting of 0, 1, 2, 3, 4, 5, 6, and 7.

### Formulas (CY-I), (CY-II), (CY-III), (CY-IV), and (CY-V)

In some embodiments, the present disclosure includes a compound of Formula (CY-I), (CY-II), (CY-ILI), (CY-IV), or (CY-V):

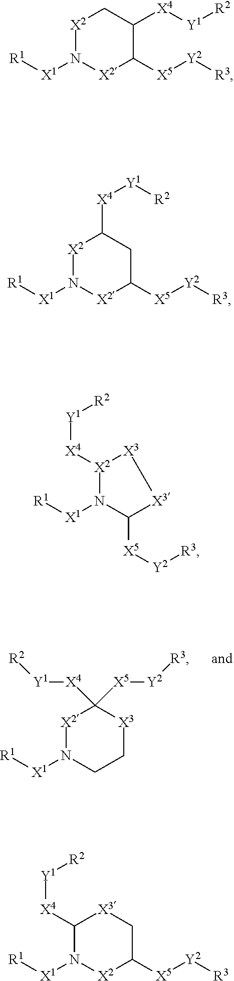

or a pharmaceutically acceptable salt thereof,
wherein X¹, X², X^{2′}, X³, X^{3′}, X⁴, X⁵, Y¹, Y², R¹, R², and R³ are defined herein.

### Formulas (CY-VI) and (CY-VII)

In some embodiments, the present disclosure includes a compound of Formula (CY-VI) or (CY-VII):

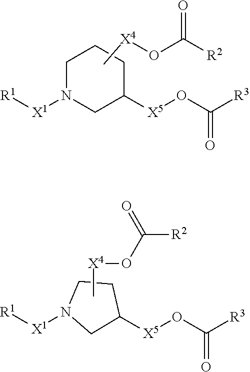

or a pharmaceutically acceptable salt thereof,
wherein X¹, X⁴, X⁵, R¹, R², and R³ are defined herein.

### Formulas (CY-VIII) and (CY-IX)

In some embodiments, the present disclosure includes a compound of Formula (CY-VIII) or (CY-IX):

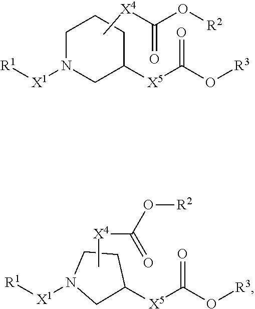

or pharmaceutically acceptable salt thereof.
wherein X¹, X⁴, X⁵, R¹, R², and R³ are defined herein.

### Formulas (CY-IV-a), (CY-IV-b), and (CY-IV-c)

In some embodiments, the present disclosure includes a compound of Formula (CY-IV-a CY-IV-b), or (CY-IV-c)

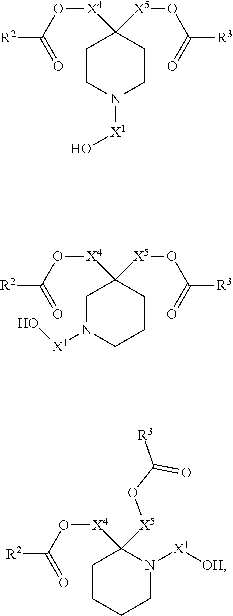

or pharmaceutically acceptable salt thereof.
wherein X¹, X⁴, X⁵, R², and R³ are defined herein.

### Formulas (CY-IV-d), (CY-IV-e), and (CY-IV-f)

In some embodiments, the present disclosure includes a compound of Formula (CY-IV-d), (CY-IV-e), or (CY-IV-f)

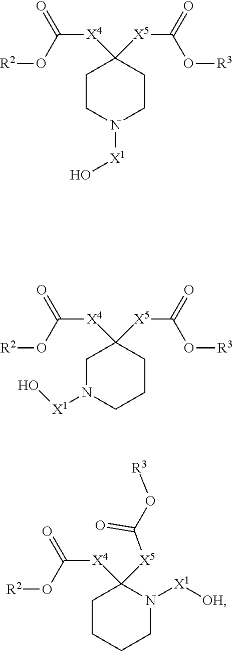

or pharmaceutically acceptable salt thereof.
wherein X¹, X⁴, X⁵, R², and R³ are defined herein.

### R¹

In some embodiments, R¹ is selected from the group consisting of —OH, —OAc, R^{1a},

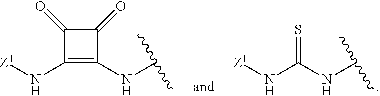

In some embodiments, R¹ is —OH or —OAc. In some embodiments, R¹ is OH. In some embodiments, R¹ is —OAc. In some embodiments, R¹ is R^{1a}. In some embodiments, R¹ is imidazolyl. In some embodiments, R¹ is

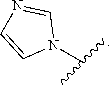 [SMILES: *n1ccnc1]

### R²

In some embodiments, R² is selected from the group consisting of optionally substituted C₄-C₂₀ alkyl, optionally substituted C₂-C₁₄ alkenyl, and —(CH₂)ₚCH(OR⁶)(OR⁷).

In some embodiments, R² is optionally substituted C₄-C₂₀ alkyl. In some embodiments, R² is optionally substituted C₅-C₁₇ alkyl. In some embodiments, R² is optionally substituted C₉-C₁₆ alkyl. In some embodiments, R² is optionally substituted C₈-C₁₀ alkyl. In some embodiments, R² is optionally substituted C₁₁-C₁₃ alkyl. In some embodiments, R² is optionally substituted C₁₄-C₁₆ alkyl. In some embodiments, R² is optionally substituted C₉ alkyl. In some embodiments, R² is optionally substituted C₁₀ alkyl. In some embodiments, R² is optionally substituted C₁₁ alkyl. In some embodiments, R² is optionally substituted C₁₂ alkyl. In some embodiments, R² is optionally substituted C₁₃ alkyl. In some embodiments, R² is optionally substituted C₁₄ alkyl. In some embodiments, R² is optionally substituted C₁₅ alkyl. In some embodiments, R² is optionally substituted C₁₆ alkyl.

In some embodiments, R² is optionally substituted C₂-C₁₄ alkenyl. In some embodiments, R² is optionally substituted C₅-C₁₄ alkenyl. In some embodiments, R² is optionally substituted C₇-C₁₄ alkenyl. In some embodiments, R² is optionally substituted C₉-C₁₄ alkenyl. In some embodiments, R² is optionally substituted C₁O-C₁₄ alkenyl. In some embodiments, R² is optionally substituted C₁₂-C₁₄ alkenyl.

In some embodiments, R² is —(CH₂)ₚCH(OR⁶)(OR⁷). In some embodiments, R² is —CH(OR⁶)(OR⁷). In some embodiments, R² is —CH₂CH(OR⁶)(OR⁷). In some embodiments, R² is —(CH₂)₂CH(OR⁶)(OR⁷). In some embodiments, R² is —(CH₂)₃CH(OR⁶)(OR⁷). In some embodiments, R² is —(CH₂)₄CH(OR⁶)(OR⁷).

In some embodiments, R² is selected from the group consisting of

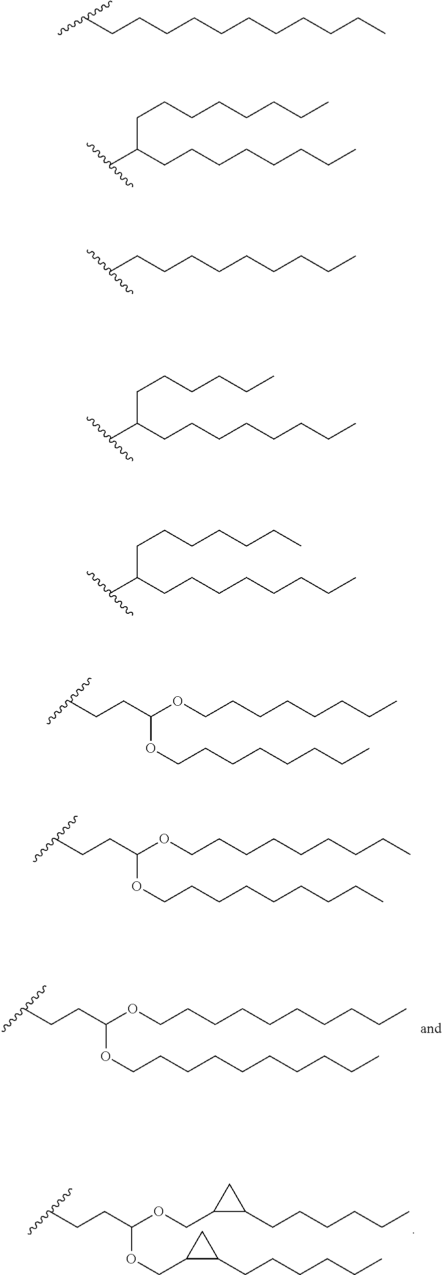

### R³

In some embodiments, R³ is selected from the group consisting of optionally substituted C₄-C₂₀ alkyl, optionally substituted C₂-C₁₄ alkenyl, and —(CH₂)_{q}CH(OR⁶)(OR⁷).

In some embodiments, R³ is optionally substituted C₄-C₂₀ alkyl. In some embodiments, R³ is optionally substituted C₅-C₁₇ alkyl. In some embodiments, R³ is optionally substituted C₉-C₁₆ alkyl. In some embodiments, R³ is optionally substituted C₅-C₁₀ alkyl. In some embodiments, R³ is optionally substituted C₁₁-C₁₃ alkyl. In some embodiments, R³ is optionally substituted C₁₄-C₁₆ alkyl. In some embodiments, R³ is optionally substituted C₉ alkyl. In some embodiments, R³ is optionally substituted C₁₀ alkyl. In some embodiments, R³ is optionally substituted C₁₁ alkyl. In some embodiments, R³ is optionally substituted C₁₂ alkyl. In some embodiments, R³ is optionally substituted C₁₃ alkyl. In some embodiments, R³ is optionally substituted C₁₄ alkyl. In some embodiments, R³ is optionally substituted C₁₅ alkyl. In some embodiments, R³ is optionally substituted C₁₆ alkyl.

In some embodiments, R³ is optionally substituted C₂-C₁₄ alkenyl. In some embodiments, R³ is optionally substituted C₅-C₁₄ alkenyl. In some embodiments, R³ is optionally substituted C₇-C₁₄ alkenyl. In some embodiments, R³ is optionally substituted C₉-C₁₄ alkenyl. In some embodiments, R³ is optionally substituted C₁O-C₁₄ alkenyl. In some embodiments, R³ is optionally substituted C₁₂-C₁₄ alkenyl.

In some embodiments, R³ is —(CH₂)_{q}CH(OR⁸)(OR⁹). In some embodiments, R³ is —CH(OR⁸)(OR⁹). In some embodiments, R³ is —CH₂CH(OR⁸)(OR⁹). In some embodiments, R³ is —(CH₂)₂CH(OR⁸)(OR⁹). In some embodiments, R³ is —(CH₂)₃CH(OR⁸)(OR⁹). In some embodiments, R³ is —(CH₂)₄CH(ORs)(OR⁹).

In some embodiments, R³ is selected from the group consisting of

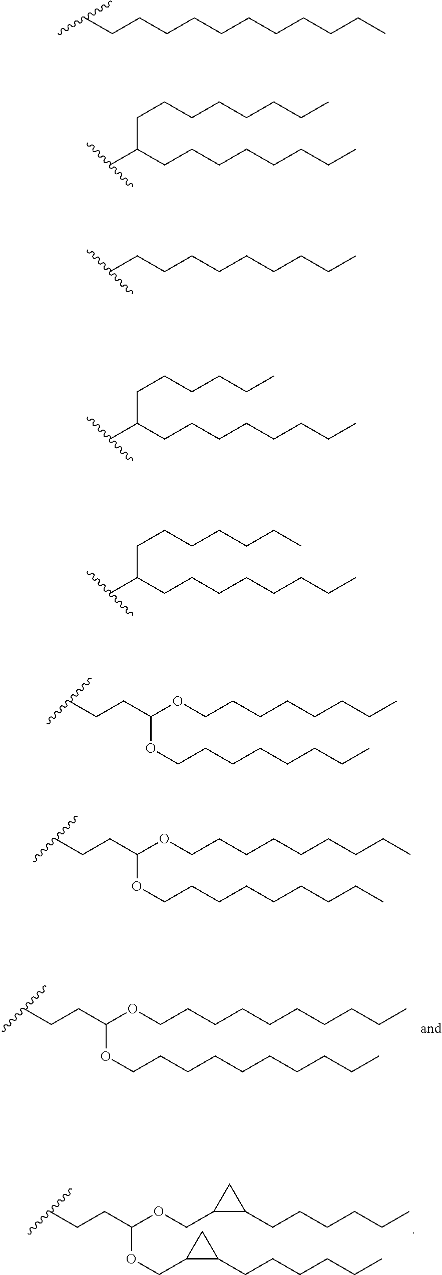

R⁶, R⁷, R⁸, R⁹

In some embodiments, R⁶, R⁷, Rs, and R⁹ are independently optionally substituted C₁-C₁₄ alkyl, optionally substituted C₂-C₁₄ alkenyl, or —(CH₂)ₘ-A-(CH₂)ₙH. In some embodiments, R⁶, R⁷, R⁸, and R⁹ are independently optionally substituted C₁-C₁₄ alkyl.

In some embodiments, R⁶, R⁷, Rs, and R⁹ are independently optionally substituted C₂-C₁₄ alkenyl. In some embodiments, R⁶, R⁷, R⁸, and R⁹ are independently —(CH₂)ₘ-A-(CH₂)ₙH.

In some embodiments, R⁶ is optionally substituted C₁-C₁₄ alkyl, optionally substituted C₂-C₁₄ alkenyl, or —(CH₂)ₘ-A-(CH₂)ₙH. In some embodiments, R⁶ is optionally substituted C₃-C₁₀ alkyl. In some embodiments, R⁶ is optionally substituted C₄-C₁₀ alkyl. In some embodiments, R⁶ is independently optionally substituted C₅-C₁₀ alkyl. In some embodiments, R⁶ is optionally substituted C₉-C₁₀ alkyl. In some embodiments, R⁶ is optionally substituted C₁-C₁₄ alkyl. In some embodiments, R⁶ is optionally substituted C₂-C₁₄ alkenyl. In some embodiments, R⁶ is —(CH₂)ₘ-A-(CH₂)ₙH.

In some embodiments, R⁷ is optionally substituted C₁-C₁₄ alkyl, optionally substituted C₂-C₁₄ alkenyl, or —(CH₂)ₘ-A-(CH₂)ₙH. In some embodiments, R⁷ is optionally substituted C₃-C₁₀ alkyl. In some embodiments, R⁷ is optionally substituted C₄-C₁₀ alkyl. In some embodiments, R⁷ is optionally substituted C₅-C₁₀ alkyl. In some embodiments, R⁷ is optionally substituted C₉-C₁₀ alkyl. In some embodiments, R⁷ is optionally substituted C₁-C₁₄ alkyl. In some embodiments, R⁷ is optionally substituted optionally substituted C₂-C₁₄ alkenyl. In some embodiments, R⁷ is —(CH₂)ₘ-A-(CH₂)ₙH.

In some embodiments, R⁸ is optionally substituted C₁-C₁₄ alkyl, optionally substituted C₂-C₁₄ alkenyl, or —(CH₂)ₘ-A-(CH₂)ₙH. In some embodiments, R⁸ is optionally substituted C₃-C₁₀ alkyl. In some embodiments, R⁸ is optionally substituted C₄-C₁₀ alkyl. In some embodiments, R⁸ is optionally substituted C₅-C₁₀ alkyl. In some embodiments, R⁸ is optionally substituted C₉-C₁₀ alkyl. In some embodiments, R⁸ is optionally substituted C₁-C₁₄ alkyl. In some embodiments, R⁸ is optionally substituted C₂-C₁₄ alkenyl. In some embodiments, R⁸ is —(CH₂)ₘ-A-(CH₂)ₙH.

In some embodiments, R⁹ is optionally substituted C₁-C₁₄ alkyl, optionally substituted C₂-C₁₄ alkenyl, or —(CH₂)ₘ-A-(CH₂)ₙH. In some embodiments, R⁹ is optionally substituted C₃-C₁₀ alkyl. In some embodiments, R⁹ is optionally substituted C₄-C₁₀ alkyl. In some embodiments, R⁹ is optionally substituted C₅-C₁₀ alkyl. In some embodiments, R⁹ is optionally substituted C₉-C₁₀ alkyl. In some embodiments, R⁹ is optionally substituted C₁-C₁₄ alkyl. In some embodiments, R⁹ is optionally substituted C₂-C₁₄ alkenyl. In some embodiments, R⁹ is —(CH₂)ₘ-A-(CH₂)ₙH.

In some embodiments, each m is independently 0, 1, 2, 3, 4, 5, 6, 7, 8, 9, 10, 11, or 12. In some embodiments, each m is 0. In some embodiments, each m is 1. In some embodiments, each m is 2. In some embodiments, each m is 3. In some embodiments, each m is 4. In some embodiments, each m is 5. In some embodiments, each m is 6. In some embodiments, each m is 7. In some embodiments, each m is 8. In some embodiments, each m is 9. In some embodiments, each m is 10. In some embodiments, each m is 11. In some embodiments, each m is 12.

In some embodiments, each n is independently 0, 1, 2, 3, 4, 5, 6, 7, 8, 9, 10, 11, or 12. In some embodiments, each n is 0. In some embodiments, each n is 1. In some embodiments, each n is 2. In some embodiments, each n is 3. In some embodiments, each n is 4. In some embodiments, each n is 5. In some embodiments, each n is 6. In some embodiments, each n is 7. In some embodiments, each n is 8. In some embodiments, each n is 9. In some embodiments, each n is 10. In some embodiments, each n is 11. In some embodiments, each n is 12.

In some embodiments, each A is independently a C₃-C₈ cycloalkylenyl. In some embodiments, each A is cyclopropylenyl.

### X¹

In some embodiments, X¹ is optionally substituted C₂-C₆ alkylenyl. In some embodiments, X¹ is optionally substituted C₂-C₅ alkylenyl. In some embodiments, X¹ is optionally substituted C₂-C₄ alkylenyl. In some embodiments, X¹ is optionally substituted C₂-C₃ alkylenyl. In some embodiments, X¹ is optionally substituted C₂ alkylenyl. In some embodiments, X¹ is optionally substituted C₃ alkylenyl. In some embodiments, X¹ is optionally substituted C₄ alkylenyl. In some embodiments, X¹ is optionally substituted C₅ alkylenyl. In some embodiments, X¹ is optionally substituted C₆ alkylenyl. In some embodiments, X¹ is optionally substituted —(CH₂)₂—. In some embodiments, X¹ is optionally substituted —(CH₂)₃—. In some embodiments, X¹ is optionally substituted —(CH₂)₄—. In some embodiments, X¹ is optionally substituted —(CH₂)₅—. In some embodiments, X¹ is optionally substituted —(CH₂)₆—.

### X²

In some embodiments, X² is selected from the group consisting of a bond, —CH₂— and —CH₂CH₂—. In some embodiments, X² is a bond. In some embodiments, X² is —CH₂—.

In some embodiments, X² is —CH₂CH₂—.

### X²

In some embodiments, X^{2′} is selected from the group consisting of a bond, —CH₂— and —CH₂CH₂—. In some embodiments, X^{2′} is a bond. In some embodiments, X^{2′} is —CH₂—. In some embodiments, X^{2′} is —CH₂CH₂—.

### X³

In some embodiments, X³ is selected from the group consisting of a bond, —CH₂— and —CH₂CH₂—. In some embodiments, X³ is a bond. In some embodiments, X³ is —CH₂—. In some embodiments, X³ is —CH₂CH₂—.

### X^{3′}

In some embodiments, X^{3′} is selected from the group consisting of a bond, —CH₂— and —CH₂CH₂—. In some embodiments, X^{3′} is a bond. In some embodiments, X^{3′} is —CH₂—. In some embodiments, X^{3′} is —CH₂CH₂—.

### X⁴

In some embodiments, X⁴ is selected from the group consisting of optionally substituted C₂-C₁₄ alkylenyl and optionally substituted C₂-C₁₄ alkenylenyl. In some embodiments, X⁴ is optionally substituted C₂-C₁₄ alkylenyl. In some embodiments, X⁴ is optionally substituted C₂-C₁₀ alkylenyl. In some embodiments, X⁴ is optionally substituted C₂-C₈ alkylenyl. In some embodiments, X⁴ is optionally substituted C₂-C₆ alkylenyl. In some embodiments, X⁴ is optionally substituted C₃-C₆ alkylenyl. In some embodiments, X⁴ is optionally substituted C₃ alkylenyl. In some embodiments, X⁴ is optionally substituted C₄ alkylenyl. In some embodiments, X⁴ is optionally substituted C₅ alkylenyl. In some embodiments, X⁴ is optionally substituted C₆ alkylenyl. In some embodiments, X⁴ is optionally substituted —(CH₂)₂—. In some embodiments, X⁴ is optionally substituted —(CH₂)₃—. In some embodiments, X⁴ is optionally substituted —(CH₂)₄—. In some embodiments, X⁴ is optionally substituted —(CH₂)₅—. In some embodiments, X⁴ is optionally substituted —(CH₂)₆—.

### X⁵

In some embodiments, X⁵ is selected from the group consisting of optionally substituted C₂-C₁₄ alkylenyl and optionally substituted C₂-C₁₄ alkenylenyl. In some embodiments, X⁵ is optionally substituted C₂-C₁₄ alkylenyl. In some embodiments, X⁵ is optionally substituted C₂-C₁₀ alkylenyl. In some embodiments, X⁵ is optionally substituted C₂-C₈ alkylenyl. In some embodiments, X⁵ is optionally substituted C₂-C₆ alkylenyl. In some embodiments, X⁵ is optionally substituted C₃-C₆ alkylenyl. In some embodiments, X⁵ is optionally substituted C₃ alkylenyl. In some embodiments, X⁵ is optionally substituted C₄ alkylenyl. In some embodiments, X⁵ is optionally substituted C₅ alkylenyl. In some embodiments, X⁵ is optionally substituted C₆ alkylenyl. In some embodiments, X⁵ is optionally substituted —(CH₂)₂—. In some embodiments, X⁵ is optionally substituted —(CH₂)₃—. In some embodiments, X⁵ is optionally substituted —(CH₂)₄—. In some embodiments, X⁵ is optionally substituted —(CH₂)₅—. In some embodiments, X⁵ is optionally substituted —(CH₂)₆—.

### Y¹

In some embodiments, Y¹ is selected from the group consisting of

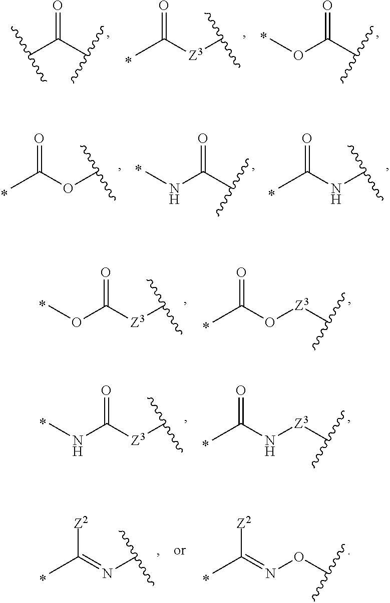

In some embodiments, Y¹ is selected from the group consisting of

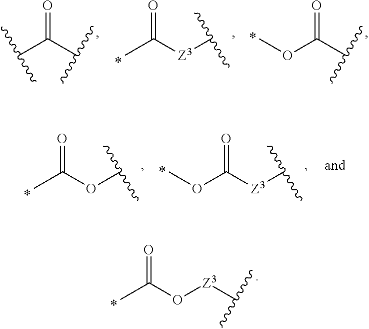

In some embodiments, Y¹ is

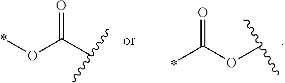

In some embodiments, Y¹ is

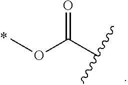 [SMILES: *C(=O)OC]

In some embodiments, Y¹ is

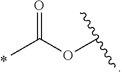 [SMILES: *OC(C)=O]

### Y²

In some embodiments, Y² is selected from the group consisting of

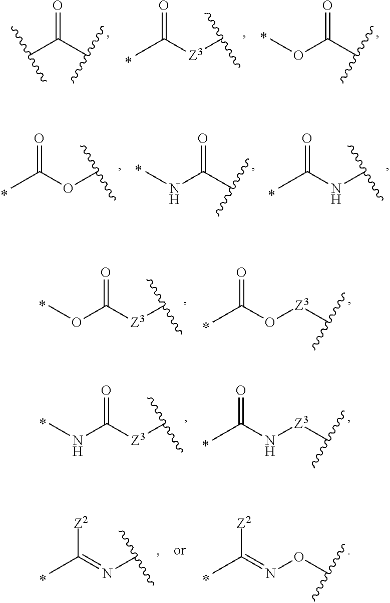

In some embodiments, Y² is selected from the group consisting of

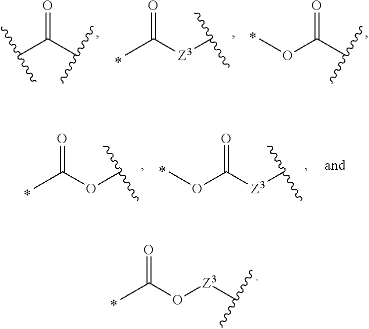

In some embodiments, Y² is

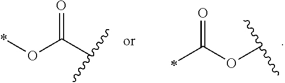

In some embodiments, Y² is

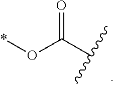 [SMILES: *OC(*)=O]

In some embodiments, Y² is

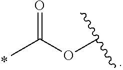 [SMILES: *OC(C)=O]

In some embodiments, Y² is

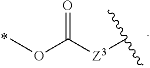

### Formula (CY-I′)

In some embodiments, Lipids of the Disclosure have a structure of Formula (CY-I′):

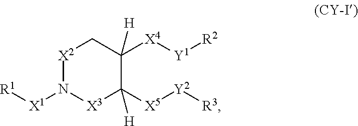

or a pharmaceutically acceptable salt thereof, wherein:
R¹ is —OH, R^{1a},

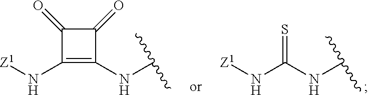

Z¹ is optionally substituted C₁-C₆ alkyl;
X¹ is optionally substituted C₁-C₆ alkylenyl;
X² and X³ are independently a bond, —CH₂—, or —CH₂CH₂—;
X⁴ and X⁵ are independently optionally substituted C₂-C₁₄ alkylenyl or optionally substituted C₂-C₁₄ alkenylenyl;
Y¹ and Y² are independently

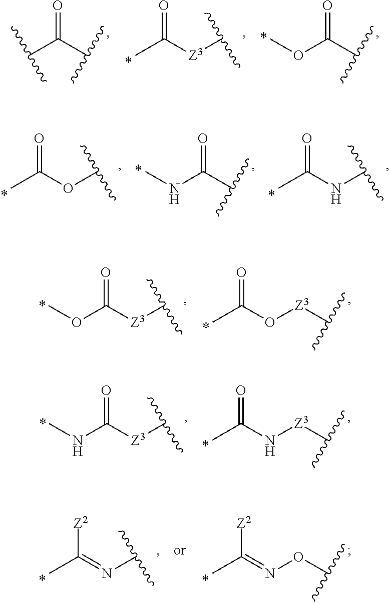

wherein the bond marked with an “*” is attached to X⁴ or X⁵;
each Z² is independently H or optionally substituted C₁-C₈ alkyl;
each Z³ is independently optionally substituted C₁-C₆ alkylenyl;
R² is optionally substituted C₄-C₂₀ alkyl, optionally substituted C₂-C₁₄ alkenyl, or —CH(OR⁶)(OR⁷),
R³ is optionally substituted C₄-C₂₀ alkyl, optionally substituted C₂-C₁₄ alkenyl, or —CH(OR⁸)(OR⁹);
R^{1a} is:

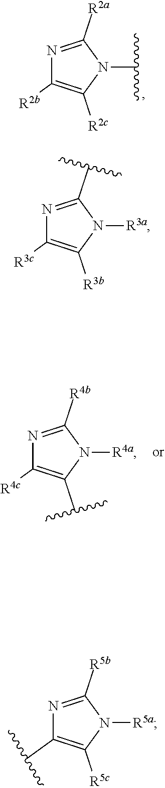

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl;
R^{5a}, R^{5b}, and R^{5c} are independently hydrogen and C₁-C₆ alkyl;
   R⁶, R⁷, R⁸, and R⁹ are independently optionally substituted C₁-C₁₄ alkyl, optionally substituted C₂-C₁₄ alkenyl, or —(CH₂)ₘ-A-(CH₂)ₙH;
   A is a C₃-C₈ cycloalkylenyl;
   each m is independently 0, 1, 2, 3, 4, 5, 6, 7, 8, 9, 10, 11, or 12; and
   each n is independently 0, 1, 2, 3, 4, 5, 6, 7, 8, 9, 10, 11, or 12.

In some embodiments, Lipids of the Disclosure have a structure of Formula (CY-I′), wherein:
R¹ is —OH, R^{1a},

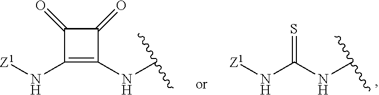

wherein Z is optionally substituted C₁-C₆ alkyl;
X¹ is optionally substituted C₂-C₆ alkylenyl;
X² and X³ are independently a bond, —CH₂—, or —CH₂CH₂—;
X⁴ and X⁵ are independently optionally substituted C₄-C₄ alkyenyl;
Y¹ and Y² are independently

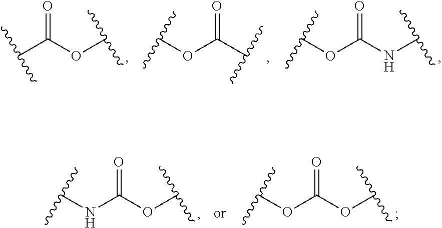 [SMILES: *OC(*)=O ; *OC(*)=O ; *NC(=O)O* ; *NC(=O)O* ; *OC(=O)O*]

R² and R³ are independently optionally substituted C₄-C₂₀ alkyl;
R^{1a} is:

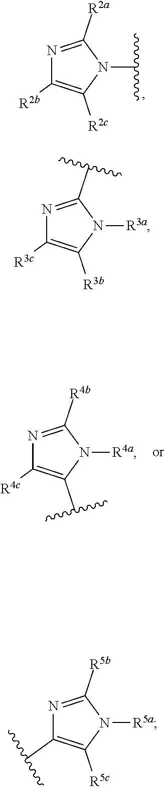

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl.

In some embodiments, Lipids of the Disclosure have a structure of Formula (CY-II′), wherein:
R¹ is —OH, R^{1a},

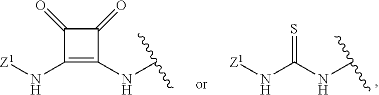

wherein Z¹ is optionally substituted C₁-C₆ alkyl;
X¹ is optionally substituted C₂-C₆ alkylenyl:
X² and X³ are independently a bond, —CH₂—, or —CH₁CH₂—;
X⁴ and X⁵ are independently optionally substituted C₂-C₁₄ alkylenyl:
Y¹ and Y² are independently

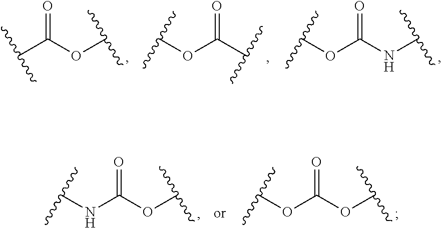 [SMILES: *OC(*)=O ; *OC(*)=O ; *NC(=O)O* ; *NC(=O)O* ; *OC(=O)O*]

R² and R³ are independently optionally substituted C₄-C₂₀ alkyl;
R^{1a} is:

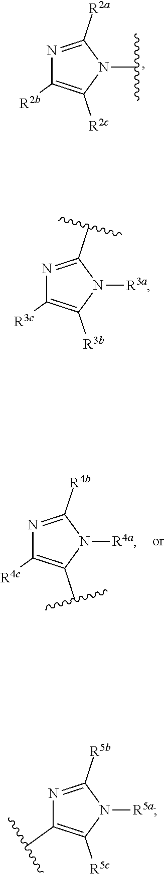

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl.

In some embodiments, Lipids of the Disclosure have a structure of Formula (CY-I′), wherein R¹ is —OH,

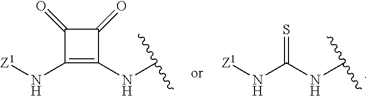

In some embodiments, Lipids of the Disclosure have a structure of Formula (CY-I′), wherein Y¹ and Y² are independently:

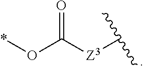

In some embodiments, Lipids of the Disclosure have a structure of Formula (CY-I′), wherein R² is —CH(OR⁶)(OR⁷).

In some embodiments, Lipids of the Disclosure have a structure of Formula (CY-I′), wherein R³ is —CH(OR⁸)(OR⁹).

Non-limiting examples of lipids having a structure of Formula (CY-I′) include compounds CY1, CY2, CY3, CY9, CY10, CY11, CY12, CY22, CY23, CY24, CY30, CY31, CY32, CY33, CY43, CY44, CY45, CY50, CY51, CY52, and CY53.

### Formula (CY-II′)

In some embodiments, Lipids of the Disclosure have a structure of Formula (CY-II′):

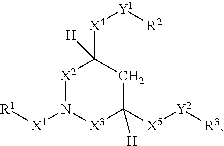

or a pharmaceutically acceptable salt thereof, wherein R¹, R², R³, X¹, X², X³, X⁴, X⁵, Y¹, and Y² are as defined in connection with Formula (CY-I′).

In some embodiments, Lipids of the Disclosure have a structure of Formula (CY-II′), wherein:
R¹ is —OH, R^{1a},

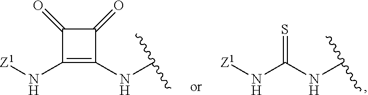

wherein Z¹ is optionally substituted C₁-C₆ alkyl;
X¹ is optionally substituted C₂-C₆ alkylenyl;
X² and X³ are independently a bond, —CH₂—, or —CH₂CH₂—;
X⁴ and X⁵ are independently optionally substituted C₂-C₁₄ alkylenyl;
Y¹ and Y² are independently

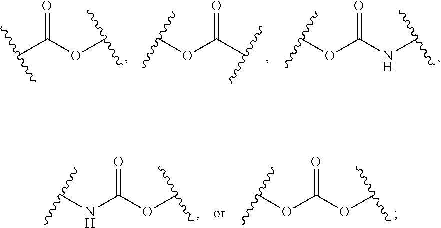 [SMILES: *OC(*)=O ; *OC(*)=O ; *NC(=O)O* ; *NC(=O)O* ; *OC(=O)O*]

R² and R³ are independently optionally substituted C₄-C₂₀ alkyl;
R^{1a} is:

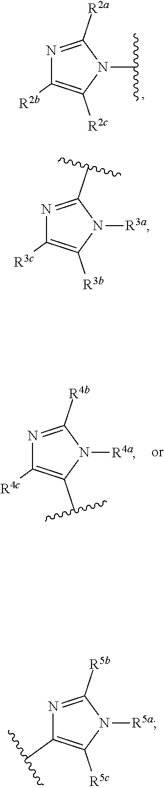

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl.

In some embodiments, Lipids of the Disclosure have a structure of Formula (CY-II′), wherein R^{a} is —OH,

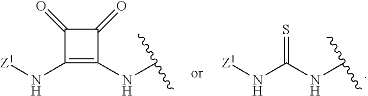

In some embodiments, Lipids of the Disclosure have a structure of Formula (CY-II′), wherein Y^{a} and Y² are independently:

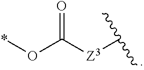

In some embodiments, Lipids of the Disclosure have a structure of Formula (CY-II′), wherein R² is —CH(OR⁶)(OR⁷).

In some embodiments, Lipids of the Disclosure have a structure of Formula (CY-II′), wherein R³ is —CH(OR⁸)(OR⁹).

Non-limiting examples of lipids having a structure of Formula (CY-II′) include compounds CY4, CY5, CY16, CY17, CY18, CY25, CY26, CY37, CY38, CY39, CY46, CY56, and CY57.

### Formula (CY-III′)

In some embodiments, Lipids of the Disclosure have a structure of Formula (CY-III′):

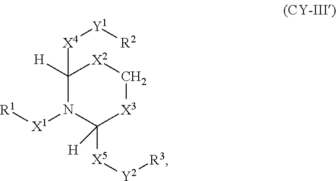

or a pharmaceutically acceptable salt thereof, wherein R¹, R², R³, X¹, X², X³, X⁴, X⁵, Y¹, and Y² are as defined in connection with Formula (CY-I′).

In some embodiments, Lipids of the Disclosure have a structure of Formula (CY-III′), wherein
R¹ is —OH, R^{1a},

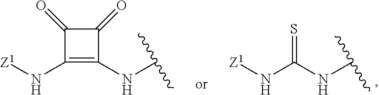

wherein Z¹ is optionally substituted C₁-C₆ alkyl;
X¹ is optionally substituted C₁-C₆ alkylenyl;
X² and X³ are independently a bond, —CH₂—, or —CH₂CH₂—;
X⁴ and X⁵ are independently optionally substituted C₂-C₁₄ alkylenyl;
Y¹ and Y² are independently

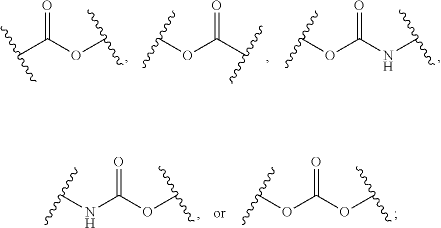 [SMILES: *OC(*)=O ; *OC(*)=O ; *NC(=O)O* ; *NC(=O)O* ; *OC(=O)O*]

R² and R³ are independently optionally substituted C₄-C₂₀ alkyl;
R^{1a} is:

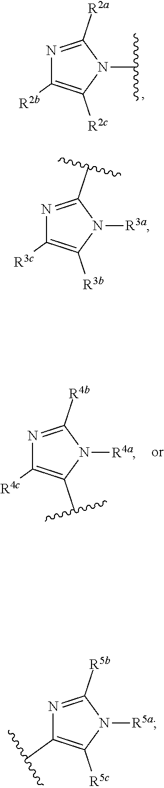

R^{2a}, R^{2b}, and R^{2c} are independently hydrogen and C₁-C₆ alkyl;
R^{3a}, R^{3b}, and R^{3c} are independently hydrogen and C₁-C₆ alkyl;
   R^{4a}, R^{4b}, and R^{4c} are independently hydrogen and C₁-C₆ alkyl; and
R^{5a}, R^{5b}, and R^{5c} are independently hydrogen and C₁-C₆ alkyl.

In some embodiments, Lipids of the Disclosure have a structure of Formula (CY-III′), wherein R¹ is —OH,

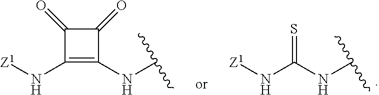

In some embodiments, Lipids of the Disclosure have a structure of Formula (CY-III′), wherein Y¹ and Y² are independently:

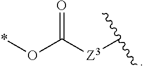

In some embodiments, Lipids of the Disclosure have a structure of Formula (CY-III′), wherein R² is —CH(OR⁶)(OR⁷).

In some embodiments, Lipids of the Disclosure have a structure of Formula (CY-III′), wherein R³ is —CH(OR⁸)(OR⁹).

Non-limiting examples of lipids having a structure of Formula (CY-III′) include CY6, CY14, CY27, CY35, CY47, and CY55.

### Formula (CY-IV′)

In some embodiments, Lipids of the Disclosure have a structure of Formula (CY-IV′):

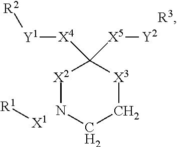

or a pharmaceutically acceptable salt thereof, wherein R¹, R², R³, X¹, X², X³, X⁴, X⁵, Y¹, and Y are as defined in connection with Formula (CY-I′).

In some embodiments, Lipids of the Disclosure have a structure of Formula (CY-IV′), wherein:
R¹ is —OH, R^{1a},

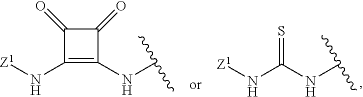

wherein Z is optionally substituted C₁-C₆ alkyl;
X¹ is optionally substituted C₂-C₆ alkylenyl;
X² and X³ are independently a bond, —CH₂—, or —CH₂CH₂—;
X⁴ and X₅ are independently optionally substituted C₂-C₁₄ alkylenyl;
Y¹ and Y² are independently

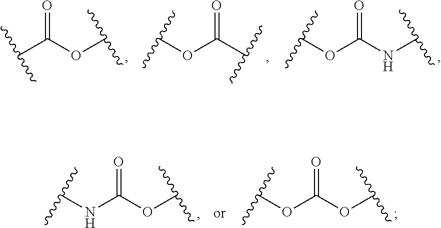 [SMILES: *OC(*)=O ; *OC(*)=O ; *NC(=O)O* ; *NC(=O)O* ; *OC(=O)O*]

R² and R³ are independently optionally substituted C₄-C₂₀ alkyl;
R^{1a} is:

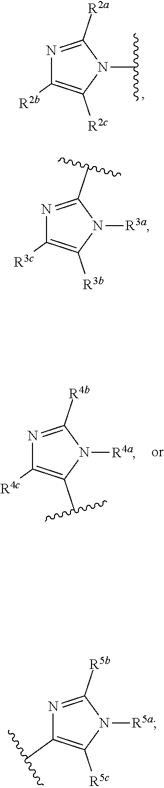

R^{2a}, R^{2b} and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl

In some embodiments, Lipids of the Disclosure have a structure of Formula (CY-IV′), wherein R¹ is —OH,

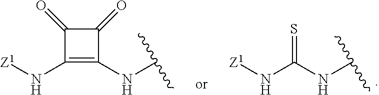

In some embodiments, Lipids of the Disclosure have a structure of Formula (CY-IV′), wherein Y¹ and Y² are independently:

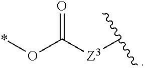

In some embodiments, Lipids of the Disclosure have a structure of Formula (CY-IV′), wherein R² is —CH(OR⁶)(OR⁷).

In some embodiments, Lipids of the Disclosure have a structure of Formula (CY-IV′), wherein R³ is —CH(OR⁸)(OR⁹).

Non-limiting examples of lipids having a structure of Formula (CY-IV′) include compounds CY7, CY8, CY19, CY20, CY21, CY28, CY29, CY40, CY41, CY42, CY48, CY49, CY58, CY59, and CY60.

### Formula (CY-V′)

In some embodiments, Lipids of the Disclosure have a structure of Formula (CY-V′):

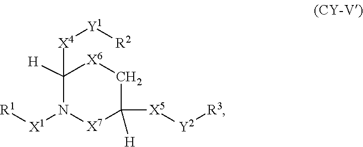

or a pharmaceutically acceptable salt thereof, wherein:
X⁶ and X⁷ are independently —CH₂— or —CH₂CH₂—; and
R¹, R², R³, X¹, X⁴, X⁵, Y¹, and Y² re as defined in connection with Formula (CY-I′).

In some embodiments, Lipids of the Disclosure have a structure of Formula (CY-V′), wherein: R¹ is —OH, R^{1a},

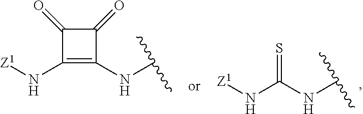

wherein Z¹ is optionally substituted C₁-C₆ alkyl;
X¹ is optionally substituted C₂-C₆ alkylenyl:
X² and X³ are independently a bond, —CH₂—, or —CH₂CH₂—;
X⁴ and X⁵ are independently optionally substituted C₂-C₁₄ alkylenyl;
Y¹ and Y² are independently

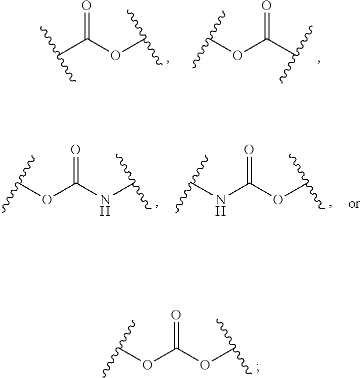 [SMILES: *OC(*)=O ; *OC(*)=O ; *NC(=O)O* ; *NC(=O)O* ; *OC(=O)O*]

R² and R³ are independently optionally substituted C₄-C₂₀ alkyl; R^{1a} is:

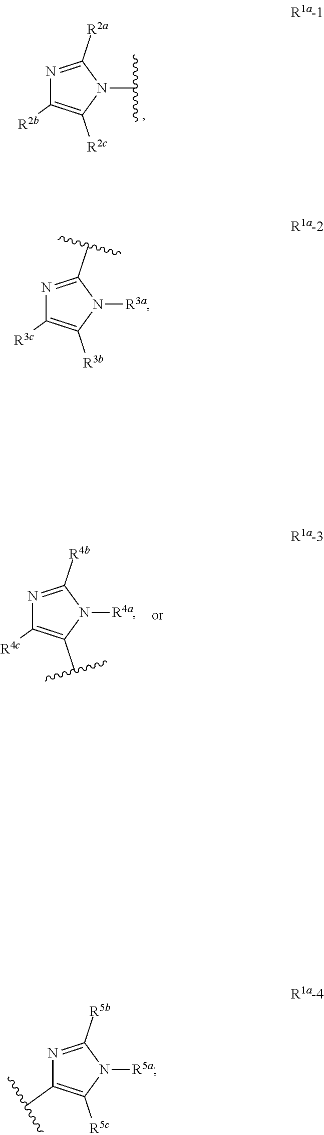

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl

In some embodiments, Lipids of the Disclosure have a structure of Formula (CY-V′), wherein Y¹ and Y² are independently:

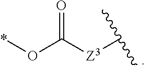

In some embodiments, Lipids of the Disclosure have a structure of Formula (CY-V′), wherein R² is —CH(OR⁶)(OR⁷).

In some embodiments, Lipids of the Disclosure have a structure of Formula (CY-V′), wherein R³ is —CH(OR⁸)(OR %.

Non-limiting examples of lipids having a structure of Formula (CY-V′) include compounds CY3, CY15, CY34, CY36, and CY54.

### Formula (CY-VI′)

In some embodiments, Lipids of the Disclosure have a structure of Formula (CY-VI′):

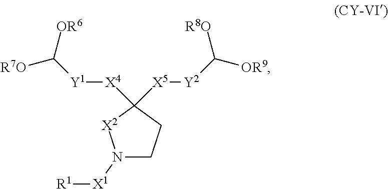

or a pharmaceutically acceptable salt thereof, wherein R¹, R⁶, R⁷, R⁸, R⁹, X¹, X², X³, X⁴, X⁵, Y¹, and Y⁷ are as defined in connection with Formula (CY-4′).

In some embodiments, Lipids of the Disclosure have a structure of Formula (CY-VI″):

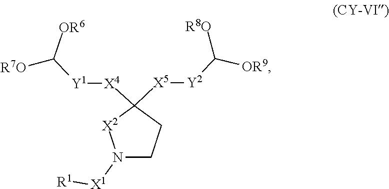

or a pharmaceutically acceptable salt thereof,
   wherein:
R¹ is selected from the group consisting of —OH, —OAc, R^{1a},

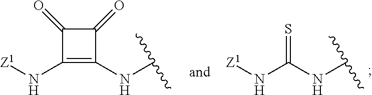

Z¹ is optionally substituted C₁-C₆ alkyl;
X¹ is optionally substituted C₂-C₆ alkylenyl;
X² is —CH₂CH₂—;
X⁴ and X⁵ are independently optionally substituted C₂-C₁₄ alkylenyl or optionally substituted C₂-C₁₄ alkenylenyl;
Y¹ and Y² are independently selected from the group consisting of

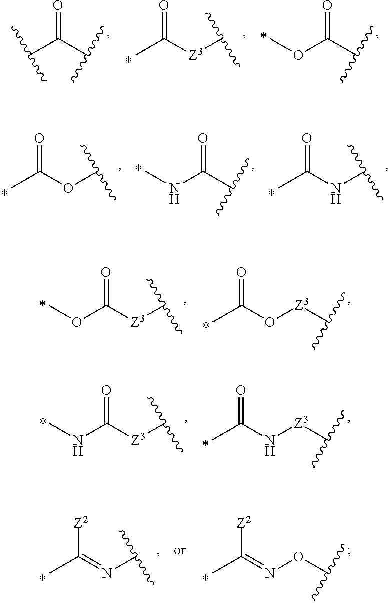

wherein the bond marked with an “*” is attached to X⁴ or X⁵;
each Z² is independently H or optionally substituted C₁-C₈ alkyl;
each Z³ is independently optionally substituted C₁-C₆ alkylenyl;
R^{1a} is:

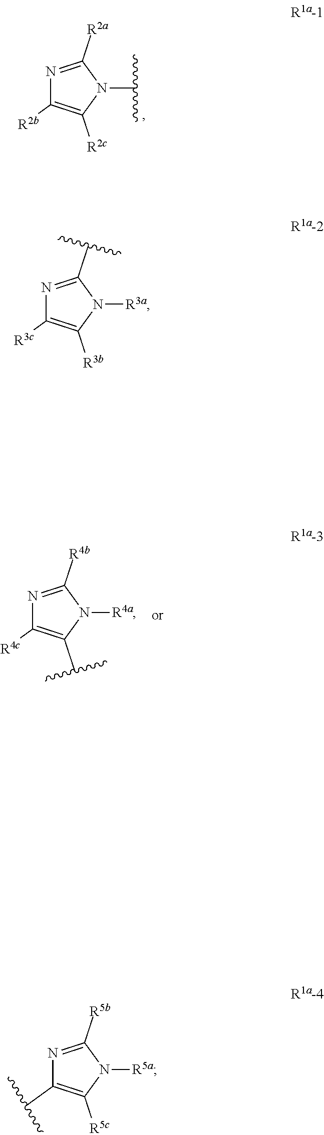

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl;
R^{5a}, R^{5b}, and R^{5c} are independently hydrogen and C₁-C₆ alkyl;
R⁶, R⁷, R⁸, and R⁹ are independently optionally substituted C₁-C₁₄ alkyl, optionally substituted C₂-C₄ alkenyl, or —(CH₂)ₘ-A-(CH₂)ₙH;
each A is independently a C₃-C₈ cycloalkylenyl;
each n is independently 0, 1, 2, 3, 4, 5, 6, 7, 8, 9, 10, 11, or 12; and
each n is independently 0, 1, 2, 3, 4, 5, 6, 7, 8, 9, 10, 11, or 12.

In some embodiments, Lipids of the Disclosure have a structure of Formula (CY-VI′), or a pharmaceutically acceptable salt thereof, wherein R¹ is —OH.

In some embodiments, Lipids of the Disclosure have a structure of Formula (CY-VI′), or a pharmaceutically acceptable salt thereof, wherein X¹ is C₂-C₆ alkylenyl.

In some embodiments, Lipids of the Disclosure have a structure of Formula (CY-VI′), or a pharmaceutically acceptable salt thereof, wherein X² is —CH₂CH₂—.

In some embodiments, Lipids of the Disclosure have a structure of Formula (CY-VI′), or a pharmaceutically acceptable salt thereof, wherein X⁴ is C₂-C₆ alkylenyl.

In some embodiments, Lipids of the Disclosure have a structure of Formula (CY-VI′), or a pharmaceutically acceptable salt thereof, wherein X⁵ is C₂-C₆ alkylenyl.

In some embodiments, Lipids of the Disclosure have a structure of Formula (CY-VI′), or a pharmaceutically acceptable salt thereof, wherein Y¹ is:

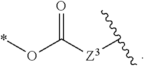

In some embodiments, Lipids of the Disclosure have a structure of Formula (CY-VI′), or a pharmaceutically acceptable salt thereof, wherein Y² is:

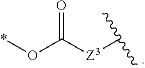

In some embodiments, Lipids of the Disclosure have a structure of Formula (CY-VI′), or a pharmaceutically acceptable salt thereof, wherein each Z³ is independently optionally substituted C₁-C₆ alkylenyl.

In some embodiments, Lipids of the Disclosure have a structure of Formula (CY-VI′), or a pharmaceutically acceptable salt thereof, wherein each Z³ is —CH₂CH₂—.

In some embodiments, Lipids of the Disclosure have a structure of Formula (CY-VI′), or a pharmaceutically acceptable salt thereof, wherein R⁶ is C₅-C₁₄ alkyl.

In some embodiments, Lipids of the Disclosure have a structure of Formula (CY-VI′), or a pharmaceutically acceptable salt thereof, wherein R⁷ is C₅-C₁₄ alkyl.

In some embodiments, Lipids of the Disclosure have a structure of Formula (CY-VI′), or a pharmaceutically acceptable salt thereof, wherein R⁶ is C₆-C₁₄ alkenyl.

In some embodiments, Lipids of the Disclosure have a structure of Formula (CY-VI′), or a pharmaceutically acceptable salt thereof, wherein R⁷ is C₆-C₁₄ alkenyl.

In some embodiments, Lipids of the Disclosure have a structure of Formula (CY-VI′), or a pharmaceutically acceptable salt thereof, wherein R⁸ is C₅-C₁₆ alkyl.

In some embodiments, Lipids of the Disclosure have a structure of Formula (CY-VI′), or a pharmaceutically acceptable salt thereof, wherein R⁹ is C₅-C₁₄ alkyl.

In some embodiments, Lipids of the Disclosure have a structure of Formula (CY-VI′), or a pharmaceutically acceptable salt thereof, wherein R⁸ is C₆-C₁₄ alkenyl.

In some embodiments, Lipids of the Disclosure have a structure of Formula (CY-VI′), or a pharmaceutically acceptable salt thereof, wherein R⁹ is C₆-C₁₄ alkenyl.

In some embodiments, Lipids of the Disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof. In some embodiments, Lipids of the Disclosure are selected from any lipid in Table (II) below or a pharmaceutically acceptable salt thereof:

TABLE (II)
Non-Limiting Examples of Ionizable Lipids with a Cyclic Core

| | Compound |
|---|---|
| Structure | No. |
| 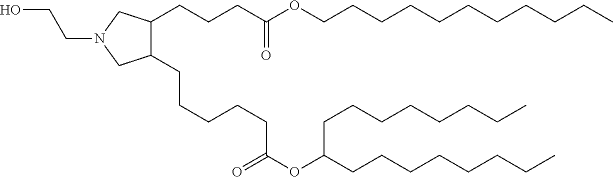 [SMILES: CCCCCCCCCCCOC(=O)CCCC1CN(CCO)CC1CCCCCC(=O)OC(CCCCCCCC)CCCCCCCC] | CY1 |
| 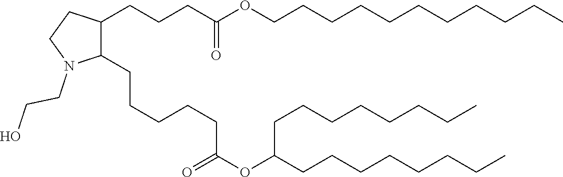 [SMILES: CCCCCCCCCCCOC(=O)CCCC1CCN(CCO)C1CCCCCC(=O)OC(CCCCCCCC)CCCCCCCC] | CY2 |
| 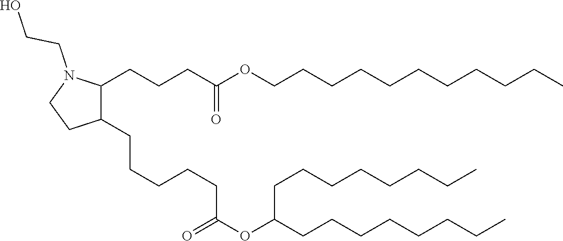 [SMILES: CCCCCCCCCCCOC(=O)CCCC1C(CCCCCC(=O)OC(CCCCCCCC)CCCCCCCC)CCN1CCO] | CY3 |
| 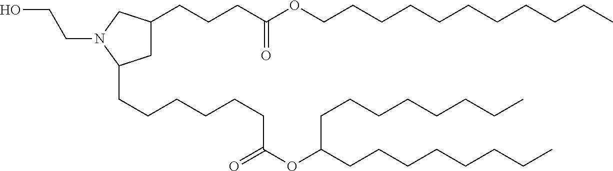 [SMILES: CCCCCCCCCCCOC(=O)CCCC1CC(CCCCCCC(=O)OC(CCCCCCCC)CCCCCCCC)N(CCO)C1] | CY4 |
| 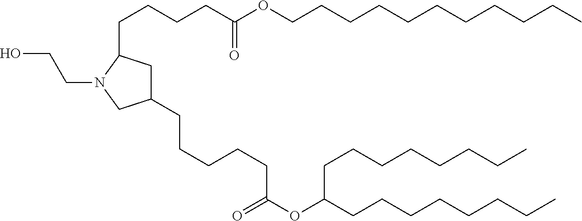 [SMILES: CCCCCCCCCCCOC(=O)CCCCC1CC(CCCCCC(=O)OC(CCCCCCCC)CCCCCCCC)CN1CCO] | CY5 |
| 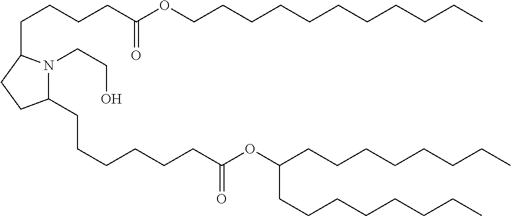 [SMILES: CCCCCCCCCCCOC(=O)CCCCC1CCC(CCCCCCC(=O)OC(CCCCCCCC)CCCCCCCC)N1CCO] | CY6 |
| 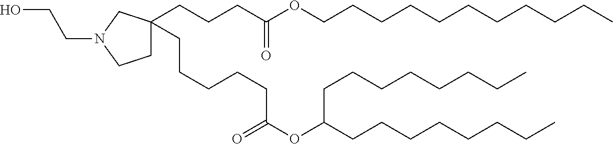 [SMILES: CCCCCCCCCCCOC(=O)CCCC1(CCCCCC(=O)OC(CCCCCCCC)CCCCCCCC)CCN(CCO)C1] | CY7 |
| 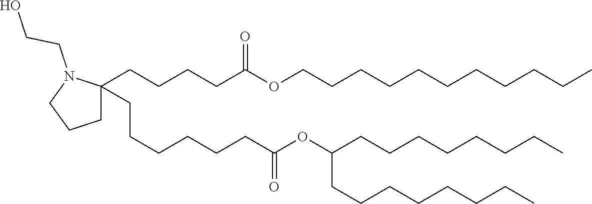 [SMILES: CCCCCCCCCCCOC(=O)CCCCC1(CCCCCCC(=O)OC(CCCCCCCC)CCCCCCCC)CCCN1CCO] | CY8 |
| 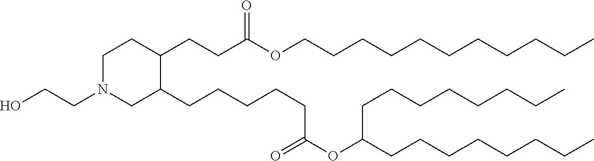 [SMILES: CCCCCCCCCCCOC(=O)CCC1CCN(CCO)CC1CCCCCC(=O)OC(CCCCCCCC)CCCCCCCC] | CY9 |
| 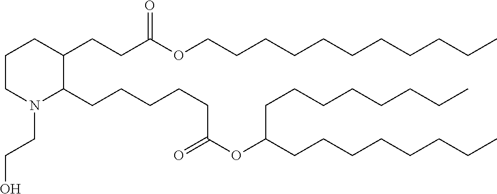 [SMILES: CCCCCCCCCCCOC(=O)CCC1CCCN(CCO)C1CCCCCC(=O)OC(CCCCCCCC)CCCCCCCC] | CY10 |
| 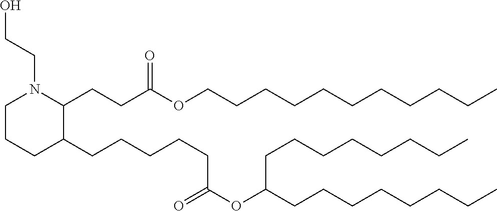 [SMILES: CCCCCCCCCCCOC(=O)CCC1C(CCCCCC(=O)OC(CCCCCCCC)CCCCCCCC)CCCN1CCO] | CY11 |
| 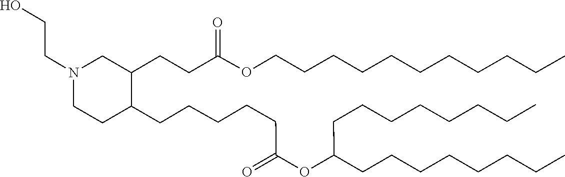 [SMILES: CCCCCCCCCCCOC(=O)CCC1CN(CCO)CCC1CCCCCC(=O)OC(CCCCCCCC)CCCCCCCC] | CY12 |
| 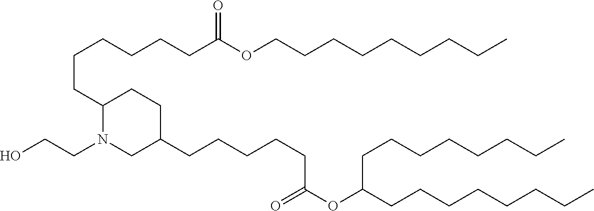 [SMILES: CCCCCCCCCOC(=O)CCCCCCC1CCC(CCCCCC(=O)OC(CCCCCCCC)CCCCCCCC)CN1CCO] | CY13 |
| 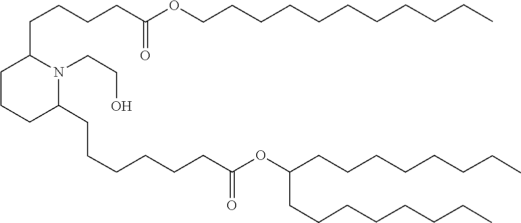 [SMILES: CCCCCCCCCCCOC(=O)CCCCC1CCCC(CCCCCCC(=O)OC(CCCCCCCC)CCCCCCCC)N1CCO] | CY14 |
| 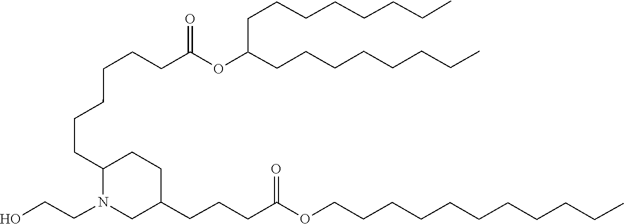 [SMILES: CCCCCCCCCCCOC(=O)CCCC1CCC(CCCCCCC(=O)OC(CCCCCCCC)CCCCCCCC)N(CCO)C1] | CY15 |
| 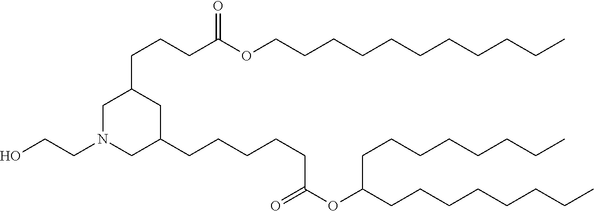 [SMILES: CCCCCCCCCCCOC(=O)CCCC1CC(CCCCCC(=O)OC(CCCCCCCC)CCCCCCCC)CN(CCO)C1] | CY16 |
| 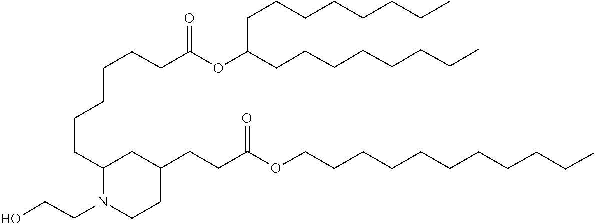 [SMILES: CCCCCCCCCCCOC(=O)CCC1CCN(CCO)C(CCCCCCC(=O)OC(CCCCCCCC)CCCCCCCC)C1] | CY17 |
| 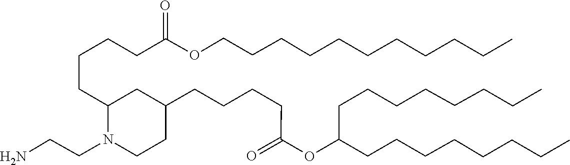 [SMILES: CCCCCCCCCCCOC(=O)CCCCC1CC(CCCCC(=O)OC(CCCCCCCC)CCCCCCCC)CCN1CCN] | CY18 |
| 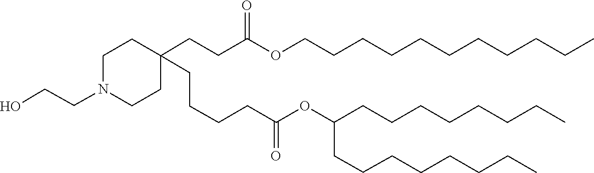 [SMILES: CCCCCCCCCCCOC(=O)CCC1(CCCCC(=O)OC(CCCCCCCC)CCCCCCCC)CCN(CCO)CC1] | CY19 |
| 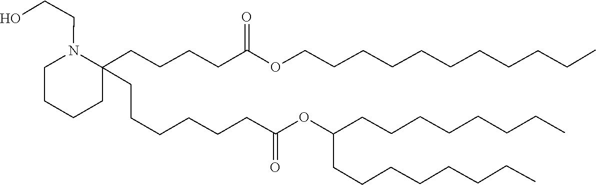 [SMILES: CCCCCCCCCCCOC(=O)CCCCC1(CCCCCCC(=O)OC(CCCCCCCC)CCCCCCCC)CCCCN1CCO] | CY20 |
| 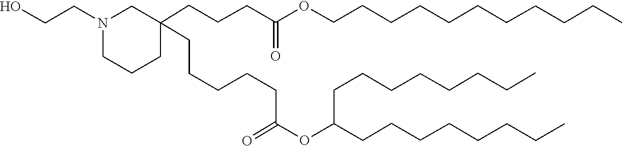 [SMILES: CCCCCCCCCCCOC(=O)CCCC1(CCCCCC(=O)OC(CCCCCCCC)CCCCCCCC)CCCN(CCO)C1] | CY21 |
| 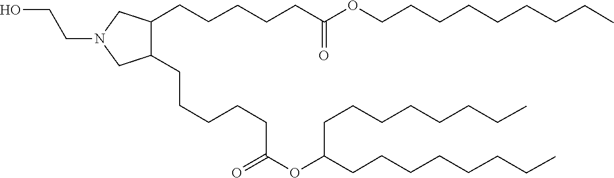 [SMILES: CCCCCCCCCOC(=O)CCCCCC1CN(CCO)CC1CCCCCC(=O)OC(CCCCCCCC)CCCCCCCC] | CY22 |
| 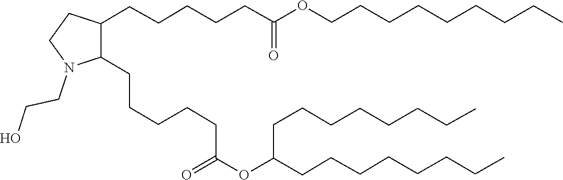 [SMILES: CCCCCCCCCOC(=O)CCCCCC1CCN(CCO)C1CCCCCC(=O)OC(CCCCCCCC)CCCCCCCC] | CY23 |
| 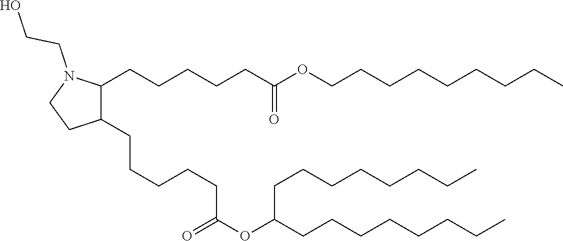 [SMILES: CCCCCCCCCOC(=O)CCCCCC1C(CCCCCC(=O)OC(CCCCCCCC)CCCCCCCC)CCN1CCO] | CY24 |
| 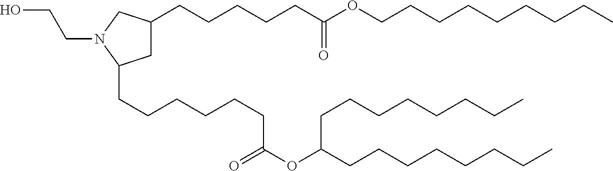 [SMILES: CCCCCCCCCOC(=O)CCCCCC1CC(CCCCCCC(=O)OC(CCCCCCCC)CCCCCCCC)N(CCO)C1] | CY25 |
| 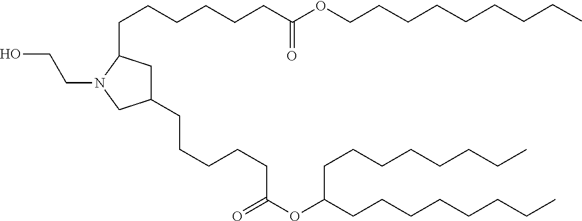 [SMILES: CCCCCCCCCOC(=O)CCCCCCC1CC(CCCCCC(=O)OC(CCCCCCCC)CCCCCCCC)CN1CCO] | CY26 |
| 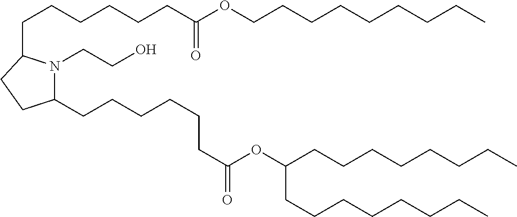 [SMILES: CCCCCCCCCOC(=O)CCCCCCC1CCC(CCCCCCC(=O)OC(CCCCCCCC)CCCCCCCC)N1CCO] | CY27 |
| 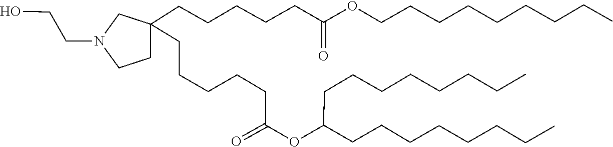 [SMILES: CCCCCCCCCOC(=O)CCCCCC1(CCCCCC(=O)OC(CCCCCCCC)CCCCCCCC)CCN(CCO)C1] | CY28 |
| 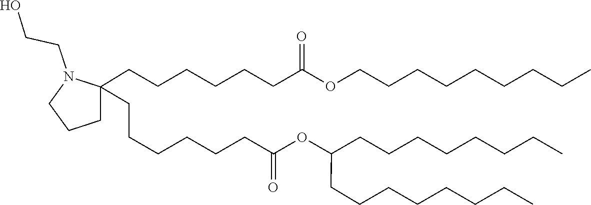 [SMILES: CCCCCCCCCOC(=O)CCCCCCC1(CCCCCCC(=O)OC(CCCCCCCC)CCCCCCCC)CCCN1CCO] | CY29 |
| 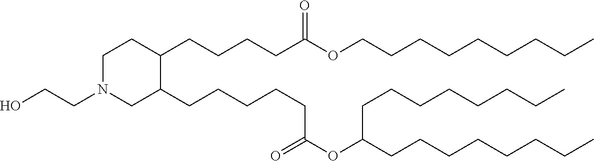 [SMILES: CCCCCCCCCOC(=O)CCCCC1CCN(CCO)CC1CCCCCC(=O)OC(CCCCCCCC)CCCCCCCC] | CY30 |
| 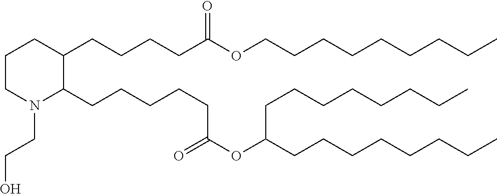 [SMILES: CCCCCCCCCOC(=O)CCCCC1CCCN(CCO)C1CCCCCC(=O)OC(CCCCCCCC)CCCCCCCC] | CY31 |
| 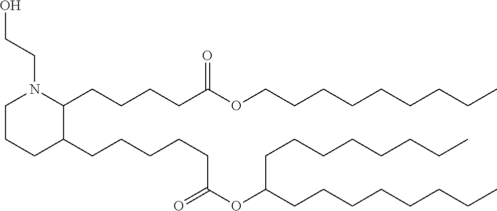 [SMILES: CCCCCCCCCOC(=O)CCCCC1C(CCCCCC(=O)OC(CCCCCCCC)CCCCCCCC)CCCN1CCO] | CY32 |
| 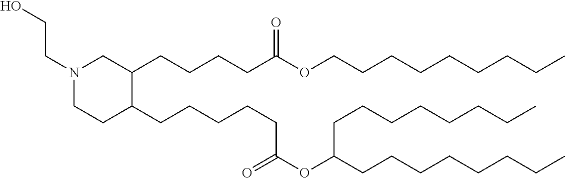 [SMILES: CCCCCCCCCOC(=O)CCCCC1CN(CCO)CCC1CCCCCC(=O)OC(CCCCCCCC)CCCCCCCC] | CY33 |
| 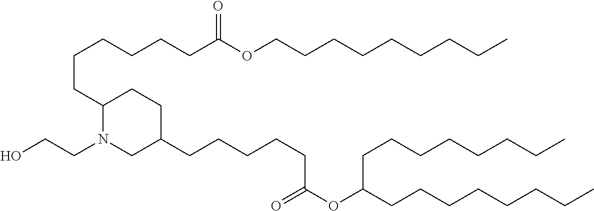 [SMILES: CCCCCCCCCOC(=O)CCCCCCC1CCC(CCCCCC(=O)OC(CCCCCCCC)CCCCCCCC)CN1CCO] | CY34 |
| 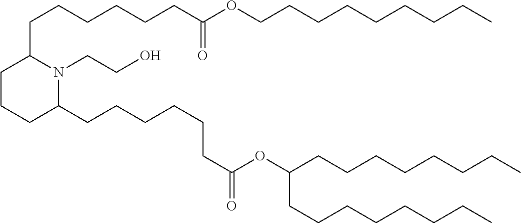 [SMILES: CCCCCCCCCOC(=O)CCCCCCC1CCCC(CCCCCCC(=O)OC(CCCCCCCC)CCCCCCCC)N1CCO] | CY35 |
| 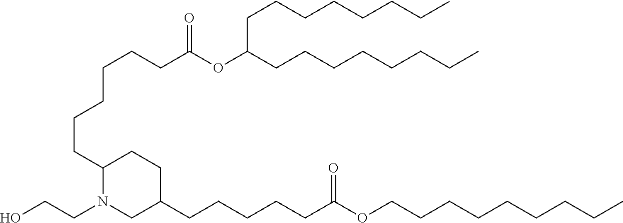 [SMILES: CCCCCCCCCOC(=O)CCCCCC1CCC(CCCCCCC(=O)OC(CCCCCCCC)CCCCCCCC)N(CCO)C1] | CY36 |
| 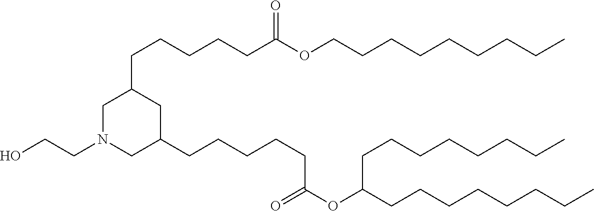 [SMILES: CCCCCCCCCOC(=O)CCCCCC1CC(CCCCCC(=O)OC(CCCCCCCC)CCCCCCCC)CN(CCO)C1] | CY37 |
| 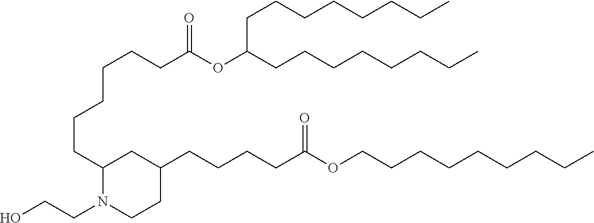 [SMILES: CCCCCCCCCOC(=O)CCCCC1CCN(CCO)C(CCCCCCC(=O)OC(CCCCCCCC)CCCCCCCC)C1] | CY38 |
| 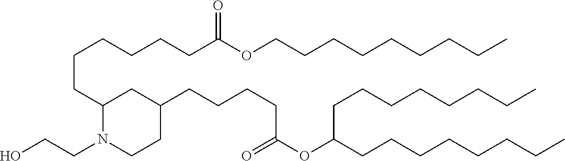 [SMILES: CCCCCCCCCOC(=O)CCCCCCC1CC(CCCCC(=O)OC(CCCCCCCC)CCCCCCCC)CCN1CCO] | CY39 |
| 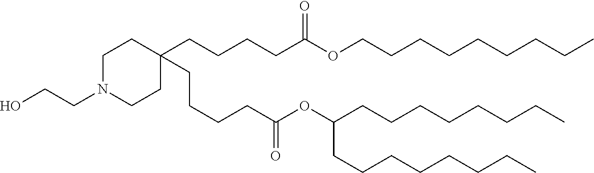 [SMILES: CCCCCCCCCOC(=O)CCCCC1(CCCCC(=O)OC(CCCCCCCC)CCCCCCCC)CCN(CCO)CC1] | CY40 |
| 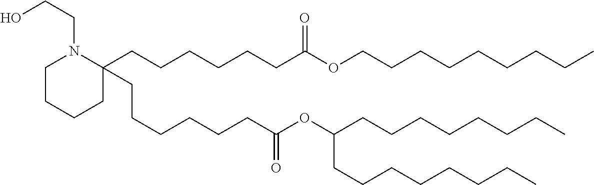 [SMILES: CCCCCCCCCOC(=O)CCCCCCC1(CCCCCCC(=O)OC(CCCCCCCC)CCCCCCCC)CCCCN1CCO] | CY41 |
| 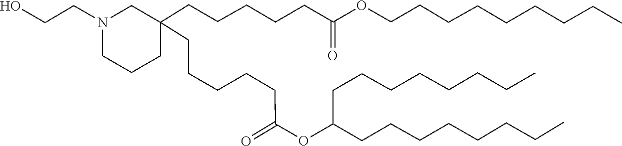 [SMILES: CCCCCCCCCOC(=O)CCCCCC1(CCCCCC(=O)OC(CCCCCCCC)CCCCCCCC)CCCN(CCO)C1] | CY42 |
| 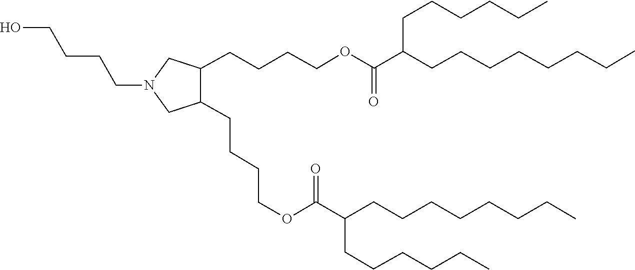 [SMILES: CCCCCCCCC(CCCCCC)C(=O)OCCCCC1CN(CCCCO)CC1CCCCOC(=O)C(CCCCCC)CCCCCCCC] | CY43 |
| 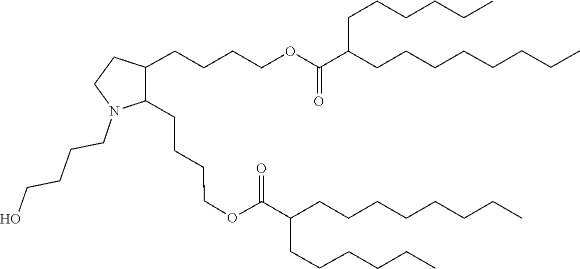 [SMILES: CCCCCCCCC(CCCCCC)C(=O)OCCCCC1CCN(CCCCO)C1CCCCOC(=O)C(CCCCCC)CCCCCCCC] | CY44 |
| 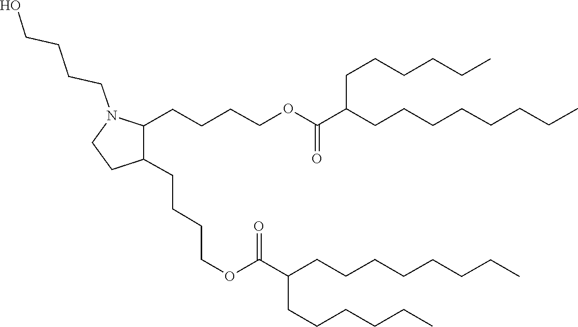 [SMILES: CCCCCCCCC(CCCCCC)C(=O)OCCCCC1CCN(CCCCO)C1CCCCOC(=O)C(CCCCCC)CCCCCCCC] | CY45 |
| 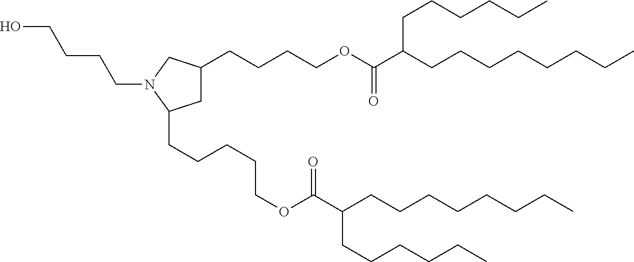 [SMILES: CCCCCCCCC(CCCCCC)C(=O)OCCCCCC1CC(CCCCOC(=O)C(CCCCCC)CCCCCCCC)CN1CCCCO] | CY46 |
| 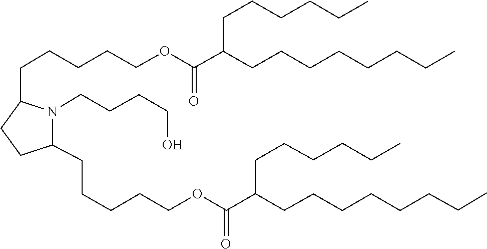 [SMILES: CCCCCCCCC(CCCCCC)C(=O)OCCCCCC1CCC(CCCCCOC(=O)C(CCCCCC)CCCCCCCC)N1CCCCO] | CY47 |
| 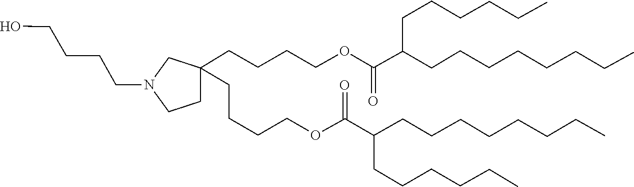 [SMILES: CCCCCCCCC(CCCCCC)C(=O)OCCCCC1(CCCCOC(=O)C(CCCCCC)CCCCCCCC)CCN(CCCCO)C1] | CY48 |
| 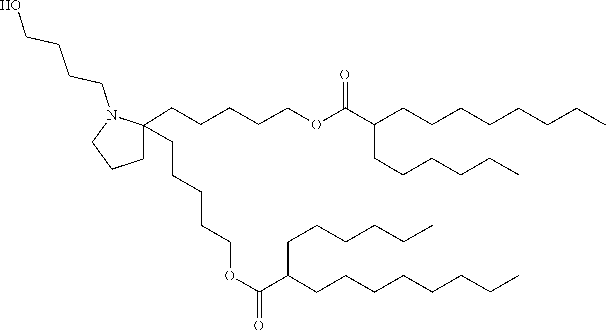 [SMILES: CCCCCCCCC(CCCCCC)C(=O)OCCCCCC1(CCCCCOC(=O)C(CCCCCC)CCCCCCCC)CCCN1CCCCO] | CY49 |
| 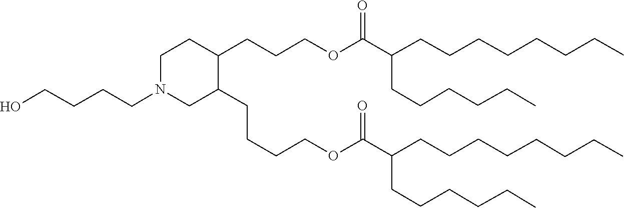 [SMILES: CCCCCCCCC(CCCCCC)C(=O)OCCCCC1CN(CCCCO)CCC1CCCOC(=O)C(CCCCCC)CCCCCCCC] | CY50 |
| 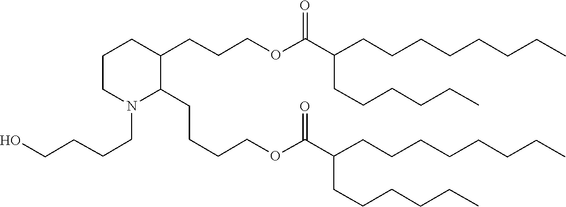 [SMILES: CCCCCCCCC(CCCCCC)C(=O)OCCCCC1C(CCCOC(=O)C(CCCCCC)CCCCCCCC)CCCN1CCCCO] | CY51 |
| 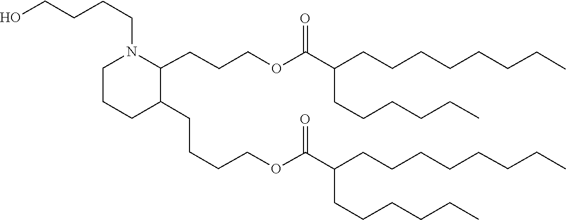 [SMILES: CCCCCCCCC(CCCCCC)C(=O)OCCCCC1CCCN(CCCCO)C1CCCOC(=O)C(CCCCCC)CCCCCCCC] | CY52 |
| 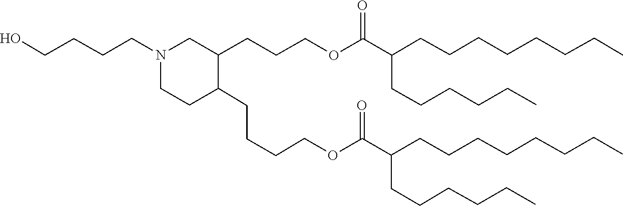 [SMILES: CCCCCCCCC(CCCCCC)C(=O)OCCCCC1CCN(CCCCO)CC1CCCOC(=O)C(CCCCCC)CCCCCCCC] | CY53 |
| 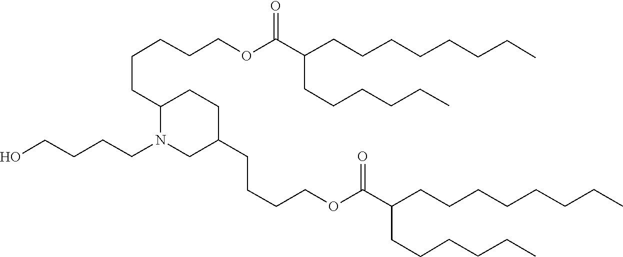 [SMILES: CCCCCCCCC(CCCCCC)C(=O)OCCCCCC1CCC(CCCCOC(=O)C(CCCCCC)CCCCCCCC)CN1CCCCO] | CY54 |
| 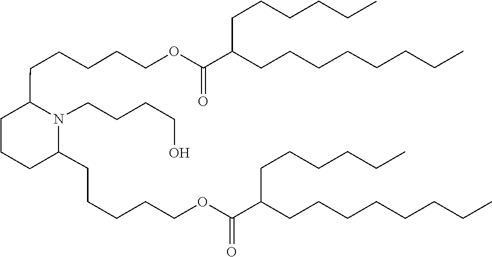 [SMILES: CCCCCCCCC(CCCCCC)C(=O)OCCCCCC1CCCC(CCCCCOC(=O)C(CCCCCC)CCCCCCCC)N1CCCCO] | CY55 |
| 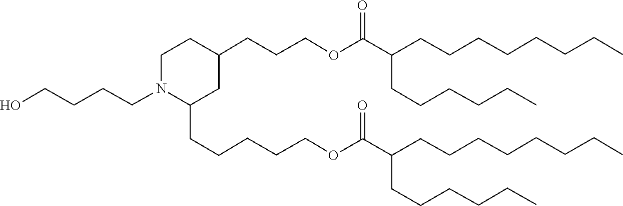 [SMILES: CCCCCCCCC(CCCCCC)C(=O)OCCCCCC1CC(CCCOC(=O)C(CCCCCC)CCCCCCCC)CCN1CCCCO] | CY56 |
| 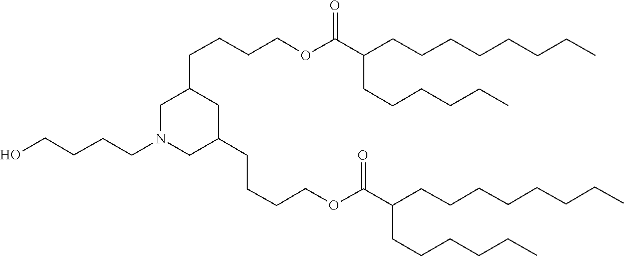 [SMILES: CCCCCCCCC(CCCCCC)C(=O)OCCCCC1CC(CCCCOC(=O)C(CCCCCC)CCCCCCCC)CN(CCCCO)C1] | CY57 |
| 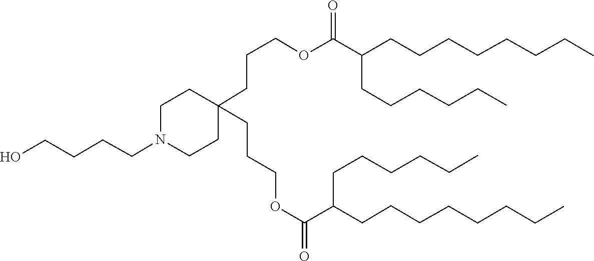 [SMILES: CCCCCCCCC(CCCCCC)C(=O)OCCCC1(CCCOC(=O)C(CCCCCC)CCCCCCCC)CCN(CCCCO)CC1] | CY58 |
| 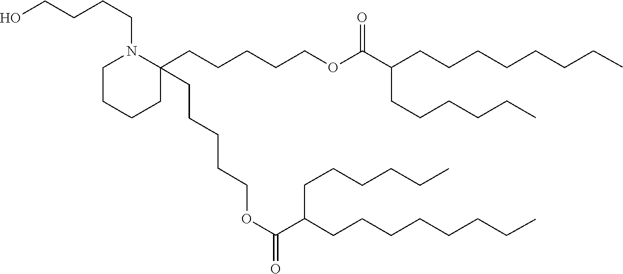 [SMILES: CCCCCCCCC(CCCCCC)C(=O)OCCCCCC1(CCCCCOC(=O)C(CCCCCC)CCCCCCCC)CCCCN1CCCCO] | CY59 |
| 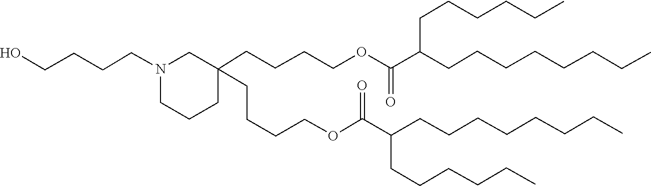 [SMILES: CCCCCCCCC(CCCCCC)C(=O)OCCCCC1(CCCCOC(=O)C(CCCCCC)CCCCCCCC)CCCN(CCCCO)C1] | CY60 |
| 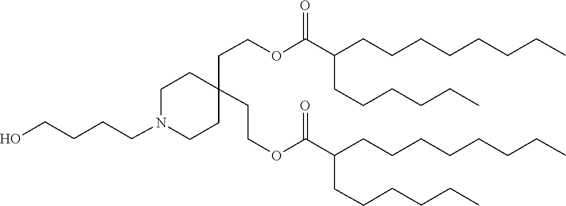 [SMILES: CCCCCCCCC(CCCCCC)C(=O)OCCC1(CCOC(=O)C(CCCCCC)CCCCCCCC)CCN(CCCCO)CC1] | CY61 |
| 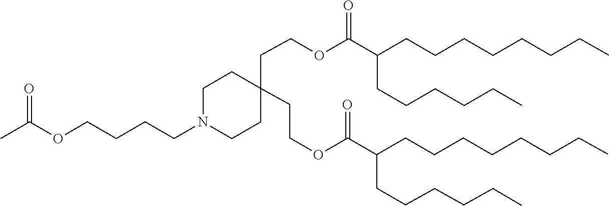 [SMILES: CCCCCCCCC(CCCCCC)C(=O)OCCC1(CCOC(=O)C(CCCCCC)CCCCCCCC)CCN(CCCCOC(C)=O)CC1] | CY62 |
| 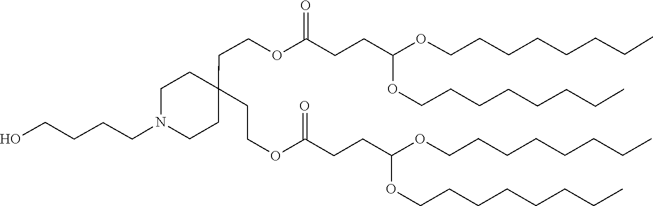 [SMILES: CCCCCCCCOC(CCC(=O)OCCC1(CCOC(=O)CCC(OCCCCCCCC)OCCCCCCCC)CCN(CCCCO)CC1)OCCCCCCCC] | CY63 |
| 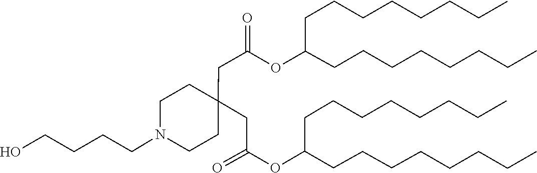 [SMILES: CCCCCCCCC(CCCCCCCC)OC(=O)CC1(CC(=O)OC(CCCCCCCC)CCCCCCCC)CCN(CCCCO)CC1] | CY64 |
| 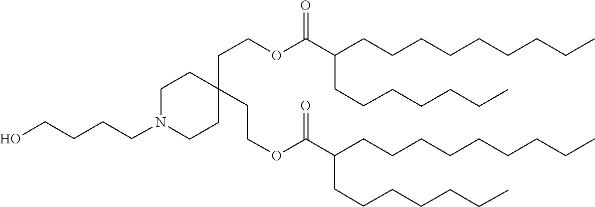 [SMILES: CCCCCCCCCC(CCCCCCC)C(=O)OCCC1(CCOC(=O)C(CCCCCCC)CCCCCCCCC)CCN(CCCCO)CC1] | CY65 |
| 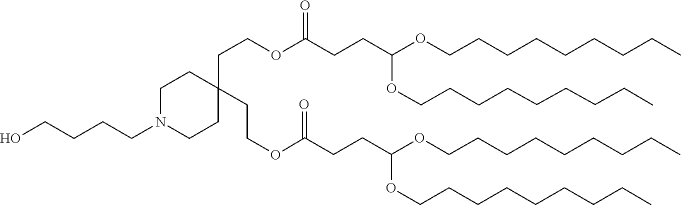 [SMILES: CCCCCCCCCOC(CCC(=O)OCCC1(CCOC(=O)CCC(OCCCCCCCCC)OCCCCCCCCC)CCN(CCCCO)CC1)OCCCCCCCCC] | CY66 |
| 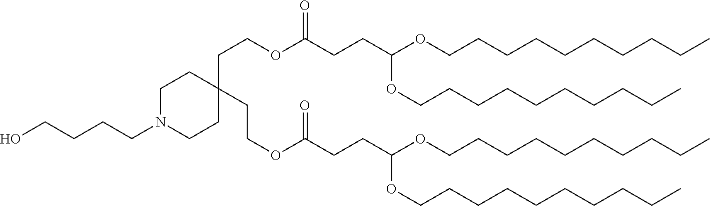 [SMILES: CCCCCCCCCCOC(CCC(=O)OCCC1(CCOC(=O)CCC(OCCCCCCCCCC)OCCCCCCCCCC)CCN(CCCCO)CC1)OCCCCCCCCCC] | CY67 |
| 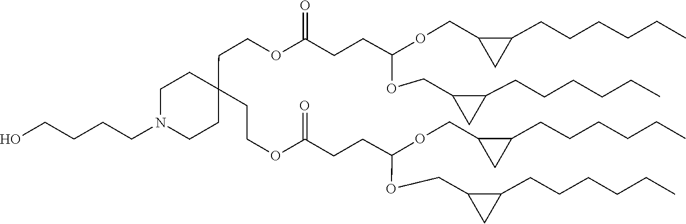 [SMILES: CCCCCCC1CC1COC(CCC(=O)OCCC1(CCOC(=O)CCC(OCC2CC2CCCCCC)OCC2CC2CCCCCC)CCN(CCCCO)CC1)OCC1CC1CCCCCC] | CY68 |
| 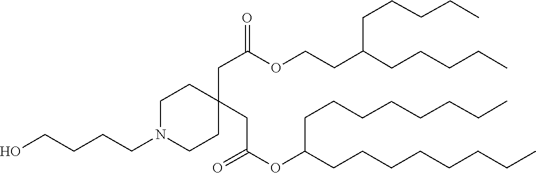 [SMILES: CCCCCCCCC(CCCCCCCC)OC(=O)CC1(CC(=O)OCCC(CCCCC)CCCCC)CCN(CCCCO)CC1] | CY69 |
| 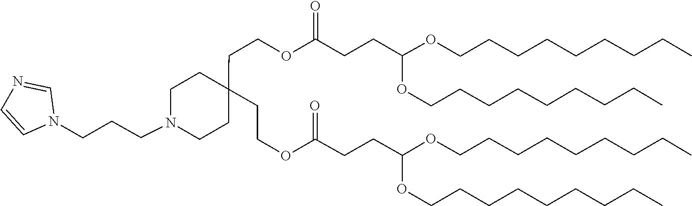 [SMILES: CCCCCCCCCOC(CCC(=O)OCCC1(CCOC(=O)CCC(OCCCCCCCCC)OCCCCCCCCC)CCN(CCCn2ccnc2)CC1)OCCCCCCCCC] | CY70 |
| 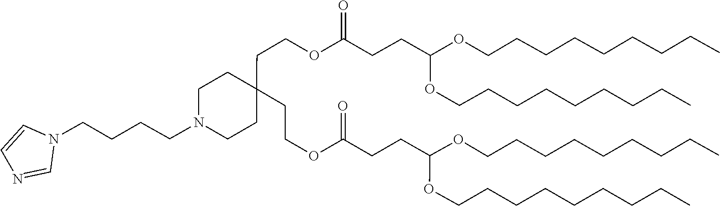 [SMILES: CCCCCCCCCOC(CCC(=O)OCCC1(CCOC(=O)CCC(OCCCCCCCCC)OCCCCCCCCC)CCN(CCCCn2ccnc2)CC1)OCCCCCCCCC] | CY71 |

In some embodiments, an LNP of the present disclosure comprises an ionizable lipid disclosed in PCT Application PCT/US2022/082276.

In one embodiment, the disclosure provides a compound of Formula IA:

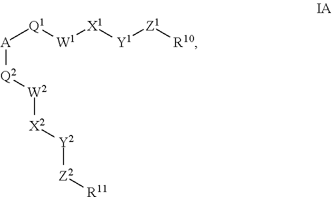

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₁-C₆ alkylenyl or —(CH₂)₂₋₆—OC(═O)—;
R¹ is selected from the group consisting of —OH,

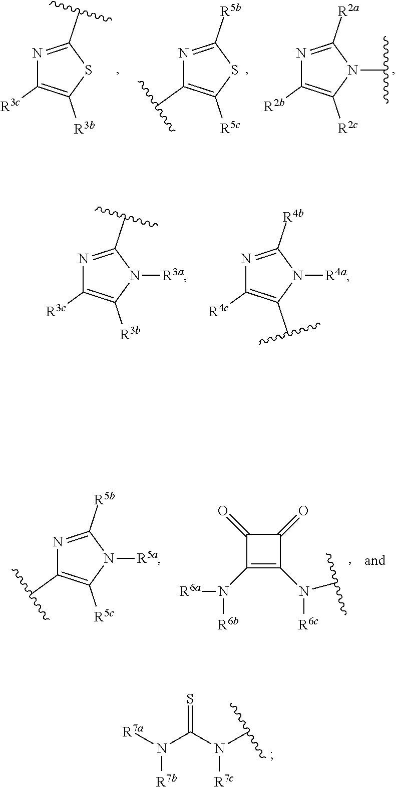

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

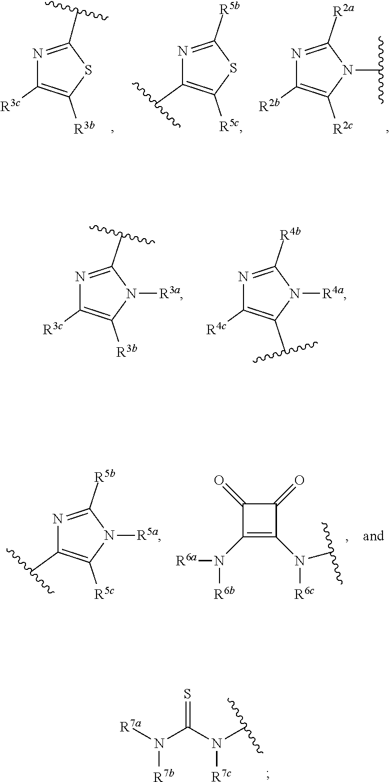

R^{9a} and R^{9b} are independently selected from the group consisting of hydrogen and C₁-C₆ alkyl; or
   R^{9a} and R^{9b} taken together with the nitrogen atom to which they are attached form a 4- to 8-membered heterocyclo;
Q¹ is C₁-C₂₀ alkylenyl:
W¹ is selected from the group consisting of —C(═O)O—, —OC(═O)—, —C(═O)N(R^{12a})—, —N(R^{12a})C(═O)—, —OC(═O)N(R²)—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6:
Z¹ is selected from the group consisting of optionally substituted C₄-C₁₂ cycloalkylenyl,

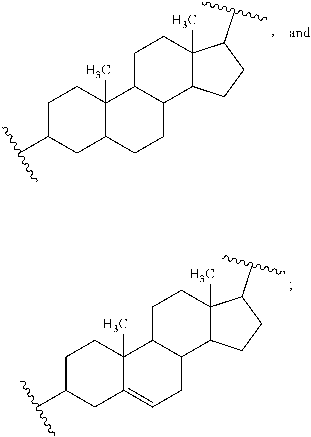 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

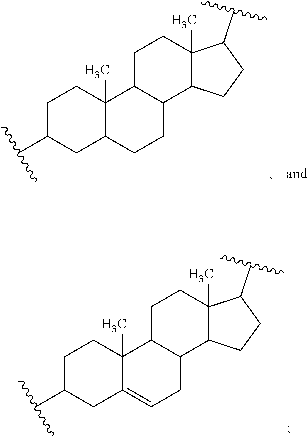 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰ and R¹¹, are optionally and independently replaced with a group selected from —O—, —CH═CH—, —S— and C₃-C₆ cycloalkylenyl.

In one embodiment, the disclosure provides a compound of Formula IB:

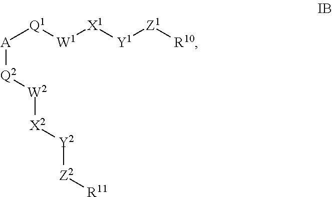

or a pharmaceutically acceptable salt or solvate thereof, wherein:
A is selected from the group consisting of —N(R^{1a})— and —C(R′)OC(═O)(R^{8c})—;
R^{1a} is -L¹-R¹;
L¹ is C₂-C₆ alkylenyl or —(CH₂)₂₋₆—OC(═O)—;
R¹ is selected from the group consisting of —OH,

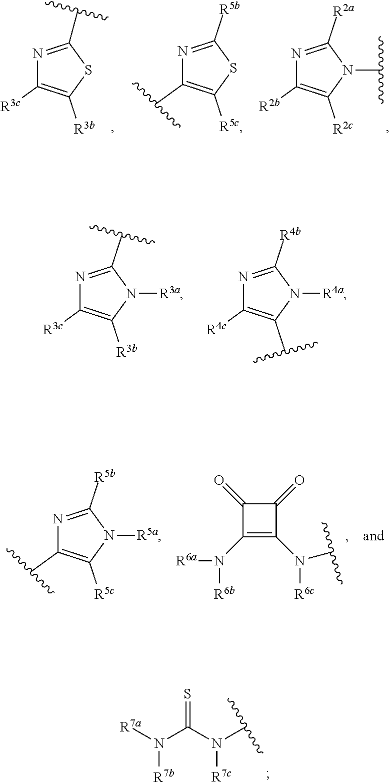

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{6c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and Roc is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and RC is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

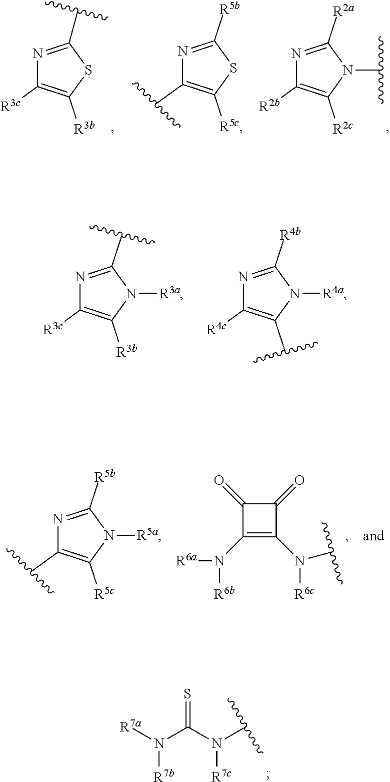

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y is selected from the group consisting of —(CH₂)ₘ—, —O—, —S—, and —S—S—;
m is 0, 1, 2, 3, 4, 5, or 6;
Z¹ is selected from the group consisting of optionally substituted C₅-C₁₂ bridged cycloalkylenyl,

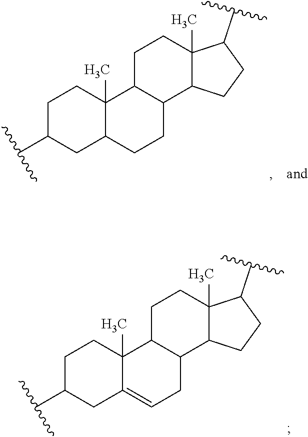 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₁-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

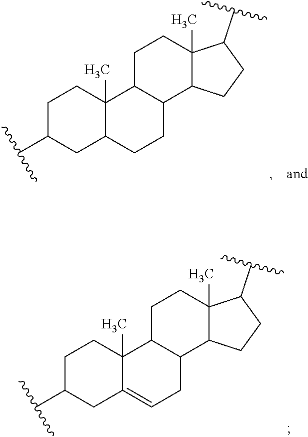 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IC:

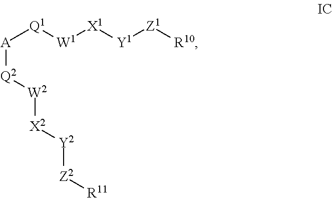

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

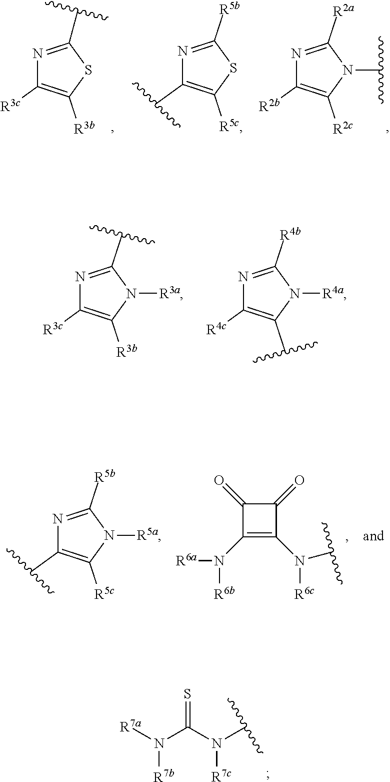

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{8c} is selected from the group consisting of hydrogen and C₁-C₆ alkyl;
R^{7a}, R^{7b} and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

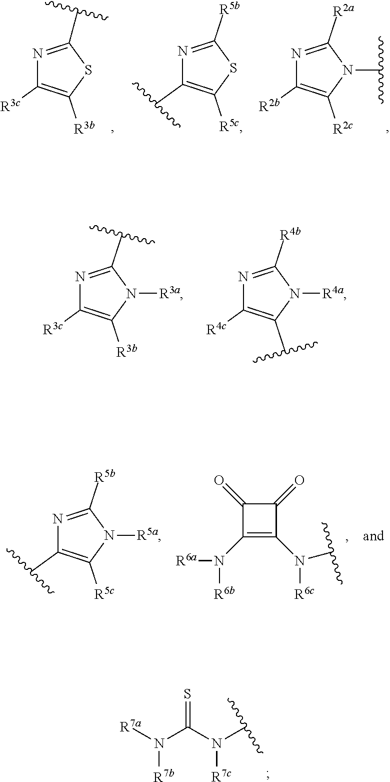

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0,1, 2, 3, 4, 5, or 6;
Z¹ is selected from the group consisting of optionally substituted C₁-C₁₂ cycloalkylenyl,

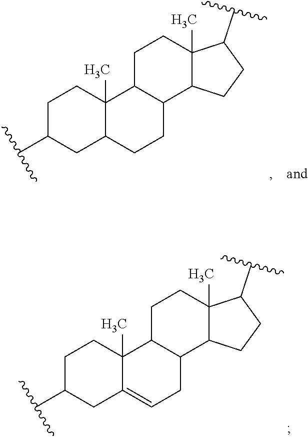 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is selected from the group consisting of —(CH₂)ₙ—, —O—, —S—, and —S—S—;
n is 0, 1, 2, 3, 4, 5, or 6;
Z² is of —(CH₂)ₚ—;
p is 0 or 1; and
R¹¹ is C₁-C₂₀branched alkyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In some embodiments, the disclosure provides a compound of any one of Formulae IA, IB, IC, or a pharmaceutically acceptable salt or solvate thereof, wherein Z¹ is optionally substituted C₅-C₁₂ bridged cycloalkylenyl.

In some embodiments, the disclosure provides a compound of any one of Formulae IA, IB, IC, or a pharmaceutically acceptable salt or solvate thereof, wherein Z¹ is not adamantyl.

In one embodiment, the disclosure provides a compound of Formula ID:

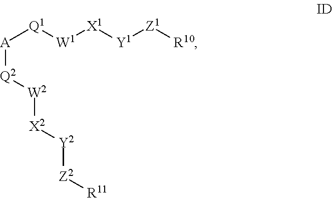

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

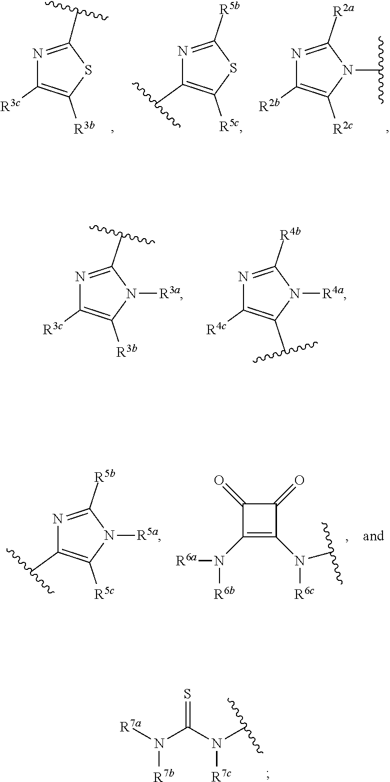

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₀ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

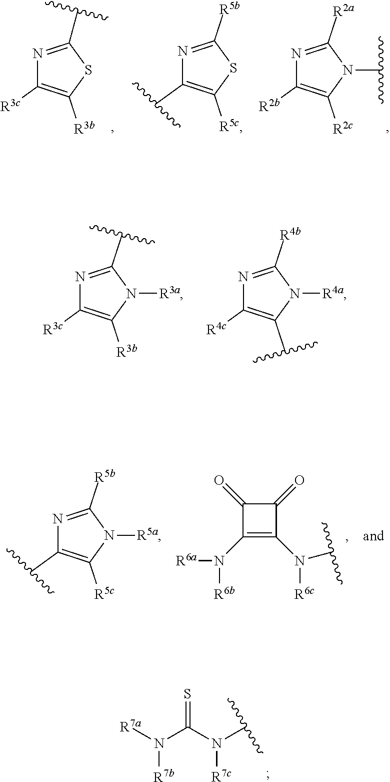

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0,1, 2, 3, 4, 5, or 6;
Z¹ is optionally substituted C₅-C₁₂ bridged cycloalkylenyl;
R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is —(CH₂)ₙ—;
n is 0, 1, 2, 3, 4, 5, or 6;
Z² is of —(CH₂)ₚ—;
p is 0 or 1; and
R¹¹ is C₁-C₂₀ branched alkyl.

In some embodiments, the disclosure provides a compound of Formula ID or a pharmaceutically acceptable salt or solvate thereof, wherein Z¹ is not adamantyl.

In one embodiment, the disclosure provides a compound of Formula I:

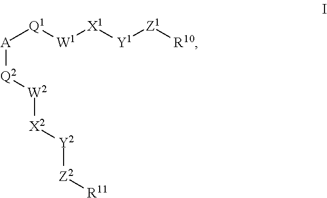

or a pharmaceutically acceptable salt or solvate thereof, wherein:
A is selected from the group consisting of —N(R^{1a})— and —C(R′)—OC(═O)(R^{8a})—;
R^{1a} is L¹-R¹;
L¹ is C₂-C₆ alkylenyl;
R¹ is selected from the group consisting of —OH,

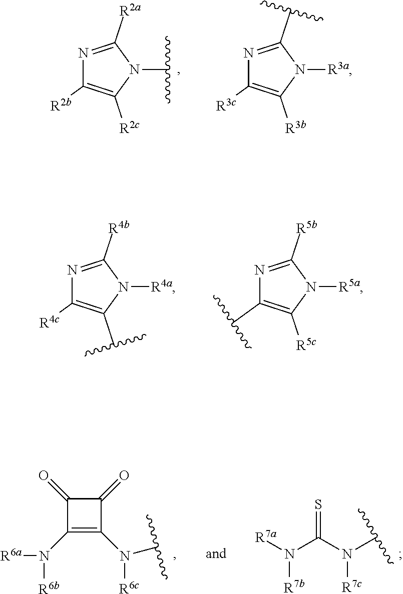

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₀ alkyl;
R′ is selected from the group consisting of hydrogen and C₁-C₆ alkyl;
R^{8a} is -L²-R⁸;
L² is C₂-C₆ alkylenyl;
R⁸ is —NR^{9a}R^{9b};
R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R′″)—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of C₄-C₁₂ cycloalkylenyl,

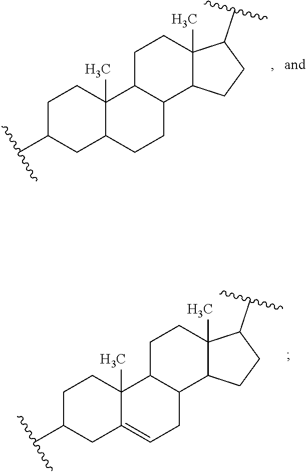 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, C₄-C₁₂ cycloalkylenyl,

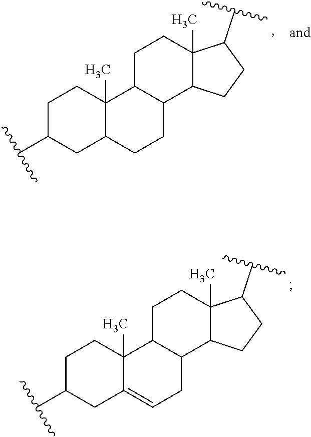 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl.

In another embodiment, the disclosure provides a compound of Formula II:

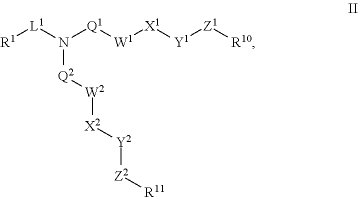

or a pharmaceutically acceptable sat or solvate thereof, wherein R¹, R¹⁰, R¹¹, Q¹, Q², W¹, W², X¹, X², Y¹, Y², Z¹, and Z² are as defined herein in Formula IA, Formula IB, Formula IC, Formula ID, Formula I, or below.

In another embodiment, the disclosure provides a compound of Formula III:

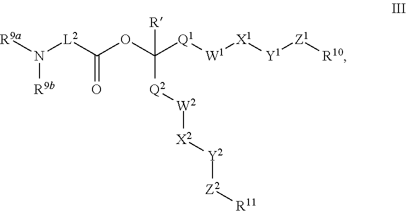

or a pharmaceutically acceptable salt or solvate thereof, wherein R′, R^{9a}, R^{9b}, R¹⁰, R¹¹, L², Q¹, Q², W¹, W, X¹, X², Y¹, Y², Z¹, and Z² are as defined herein in Formula IA, Formula IB, Formula IC, Formula ID, Formula I, or below.

In another embodiment, the disclosure provides a compound of Formula IV:

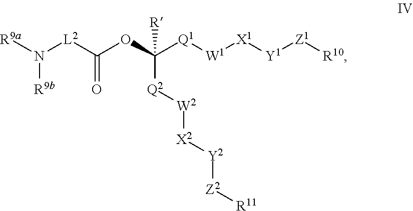

or a pharmaceutically acceptable salt or solvate thereof, wherein R′, R^{9a}, R^{9b}, R¹⁰, R¹¹, L², Q¹, Q², W¹, W², X¹, X², Y¹, Y², Z¹, and Z² are as defined herein in Formula IA, Formula IB, Formula IC, Formula ID, Formula I, or below, with the proviso that -Q¹W¹X—Y¹Z¹—R¹⁰ is not the same as -Q²-W²—X²—Y²—Z²—R¹¹, i.e., the carbon atom bearing R′ is an asymmetrical carbon atom.

In another embodiment, the disclosure provides a compound of Formula V:

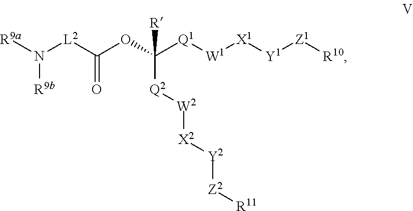

or a pharmaceutically acceptable salt or solvate thereof, wherein R′, R^{9a}, R^{9b}, R¹⁰, R¹¹, L², Q¹, Q², W¹, W², X¹, X², Y¹, Z¹, and Z² are as defined herein in Formula IA, Formula IB, Formula IC, Formula ID, Formula I, or below, with the proviso that -Q¹-W¹—X¹—Y¹—Z¹—R¹⁰ is not the same as -Q²-W²—X²—Y²—Z²—R¹¹, i.e., the carbon atom bearing R′ is an asymmetrical carbon atom.

In another embodiment, the disclosure provides a compound of Formula VI:

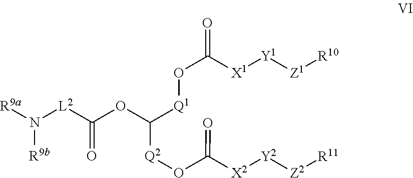

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VI′:

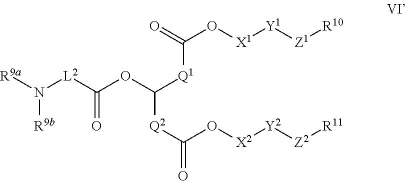

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VI″:

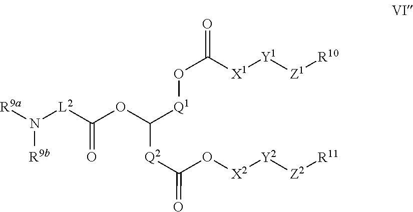

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VI′″:

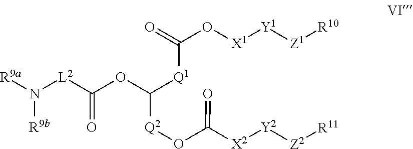

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below.

Formula IA, Formula IB, Formula IC, Formula I, In another embodiment, the disclosure provides a compound of Formula VII:

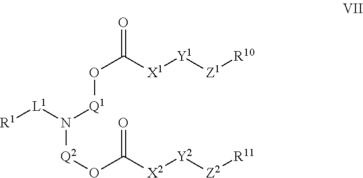

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII′:

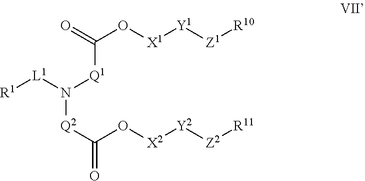

or a pharmaceutically acceptable salt or solvate thereof, wherein R¹, L¹, Q¹, Q², X¹, X², Y¹, Y², Z¹, Z², R^{1O}, an R¹¹ are as defined herein in Formula IA, Formula IB, Formula IC, Formula ID, Formula I, or below,
In another embodiment, the disclosure provides a compound of Formula VII′″:

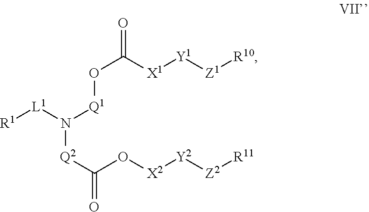

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII′″:

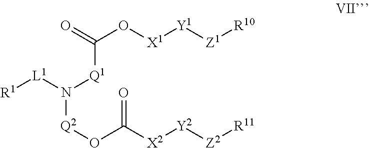

or a pharmaceutically acceptable salt or solvate thereof, wherein R¹, L¹, Q¹, Q², X¹, X², Y¹, Y², Z¹, Z², R^{1O}, an R¹¹ are as defined herein in Formula IA, Formula IB, Formula IC, Formula ID, Formula I, or below.

Formula IA, Formula IB, Formula IC, Formula I, In another embodiment, the disclosure provides a compound of Formula VIII:

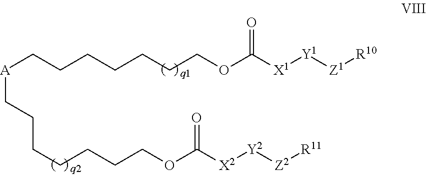

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII, wherein Z¹ is an optionally substituted C₅-C₁₂ bridged cycloalkylenyl.

In another embodiment, the disclosure provides a compound of Formula VIII′:

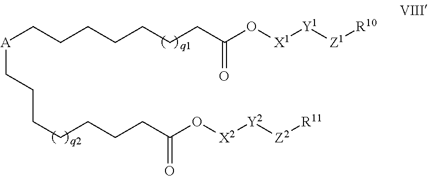

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII′, wherein Z¹ is an optionally substituted C₅-C₁₂ bridged cycloalkylenyl.

In another embodiment, the disclosure provides a compound of Formula VIII″:

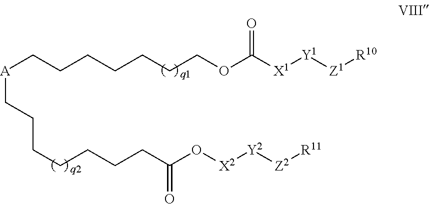

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII″, wherein Z¹ is an optionally substituted C₅-C₁₂ bridged cycloalkylenyl.

In another embodiment, the disclosure provides a compound of Formula VIII′″:

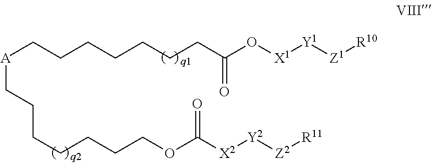

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII′″, wherein Z¹ is an optionally substituted C₅-C₁₂ bridged cycloalkylenyl.

In another embodiment, the disclosure provides a compound of Formula IX:

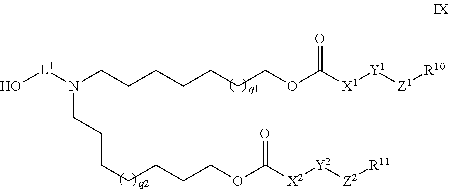

or a pharmaceutically acceptable salt or solvate thereof,
wherein
q¹ is 0, 1, 2, or 3;
q² is 0, 1, 2, or 3;
L¹, X¹, X², Y², Z¹, Z², R¹⁰, an R¹¹ are as defined herein in Formula IA, Formula IB, Formula IC, Formula ID, Formula I, or below.

In certain embodiments, the compound is a compound of Formula IX, wherein Z¹ is an optionally substituted C₅-C₁₂ bridged cycloalkylenyl.

In another embodiment, the disclosure provides a compound of Formula IX′:

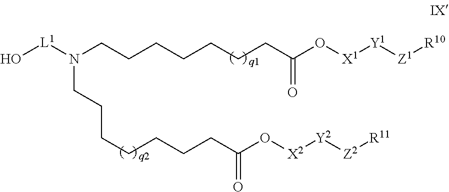

or a pharmaceutically acceptable salt or solvate thereof,
wherein
q¹ is 0, 1, 2, or
q² is 0, 1, 2, or
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX′, wherein Z¹ is an optionally substituted C₅-C₁₂ bridged cycloalkylenyl.

In another embodiment, the disclosure provides a compound of Formula IX″:

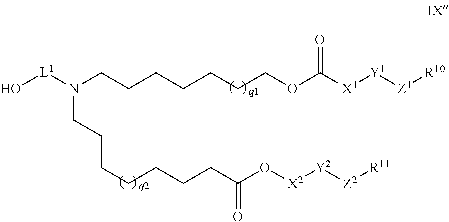

or a pharmaceutically acceptable salt or solvate thereof,
wherein
q¹ is 0, 1, 2, or 3;
q² is 0, 1, 2, or 3
L¹, X¹, X¹, Y², Z¹, Z², R¹⁰, an R¹¹ are as defined herein in Formula IA, Formula IB, Formula IC, Formula ID, Formula I, or below.

In certain embodiments, the compound is a compound of Formula IX″, wherein Z¹ is an optionally substituted C₅-C₁₂ bridged cycloalkylenyl.

In another embodiment, the disclosure provides a compound of Formula IX′″:

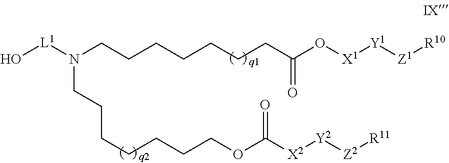

or a pharmaceutically acceptable salt or solvate thereof,
wherein
q is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX′″, wherein Z¹ is an optionally substituted C₅-C₁₂ bridged cycloalkylenyl.

In another embodiment, the disclosure provides a compound of Formula X:

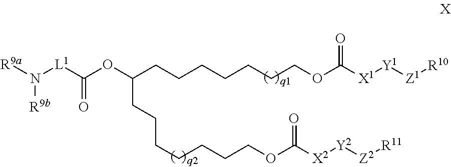

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X, wherein Z¹ is an optionally substituted C₅-C₁₂ bridged cycloalkylenyl.

In another embodiment, the disclosure provides a compound of Formula X′:

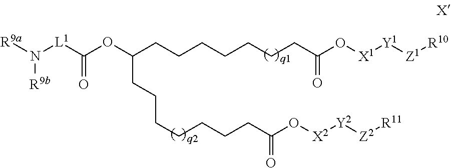

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R^{9b}, an R¹¹ are as defined herein in Formula IA, Formula IB, Formula IC, Formula ID, Formula I or below.

In certain embodiments, the compound is a compound of Formula X′, wherein Z¹ is an optionally substituted C₅-C₁₂ bridged cycloalkylenyl.

In another embodiment, the disclosure provides a compound of Formula X″:

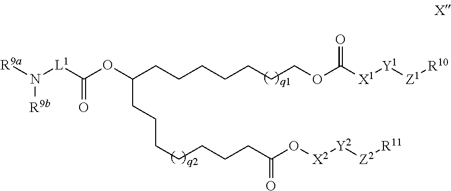

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X″, wherein Z¹ is an optionally substituted C₅-C₁₂ bridged cycloalkylenyl.

In another embodiment, the disclosure provides a compound of Formula X′″:

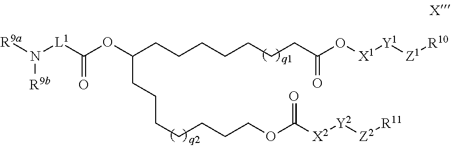

or a pharmaceutically acceptable salt or solvate thereof
wherein
q¹ is 0, 1, 2, or 3;
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X′″, wherein Z¹ is an optionally substituted C₅-C₁₂ bridged cycloalkylenyl.

In another embodiment, the disclosure provides a compound of Formula XI:

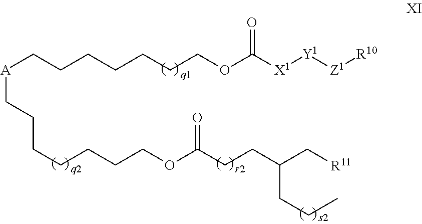

or a pharmaceutically acceptable salt or solvate thereof.
wherein
q¹ is 0, 1, 2, or 3;
q² is 0, 1, 2, or 3;
r² is 0, 1, or 2:
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI, wherein Z¹ is an optionally substituted C₅-C₁₂ bridged cycloalkylenyl.

In another embodiment, the disclosure provides a compound of Formula XI′:

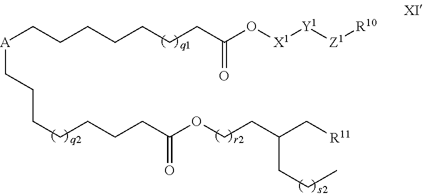

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI′, wherein Z¹ is an optionally substituted C₅-C₁₂ bridged cycloalkylenyl.

In another embodiment, the disclosure provides a compound of Formula XI″:

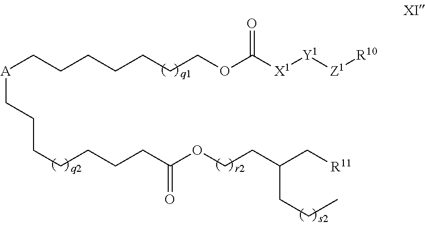

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI″, wherein Z¹ is an optionally substituted C₅-C₁₂ bridged cycloalkylenyl.

In another embodiment, the disclosure provides a compound of Formula XI′″:

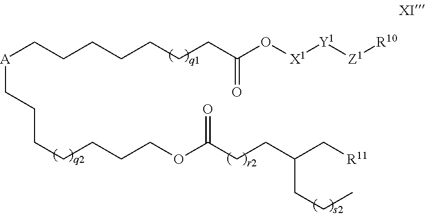

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI′″, wherein Z¹ is an optionally substituted C₅-C₁₂ bridged cycloalkylenyl.

In another embodiment, the disclosure provides a compound of Formula XII:

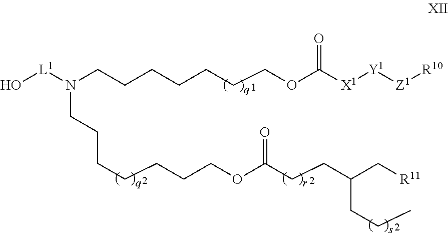

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I, or below.

In certain embodiments, the compound is a compound of Formula XII, wherein Z¹ is an optionally substituted C₅-C₁₂ bridged cycloalkylenyl.

In another embodiment, the disclosure provides a compound of Formula XII′:

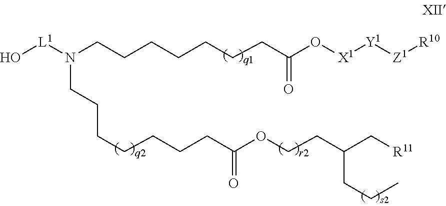

or a pharmaceutically acceptable salt or solvate thereof,
wherein
q is 0, 1, 2, or 3:
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC., Formula ID, Formula I, or below.

In certain embodiments, the compound is a compound of Formula XII′, wherein Z¹ is an optionally substituted C₅-C₁₂ bridged cycloalkylenyl.

In another embodiment, the disclosure provides a compound of Formula XII″:

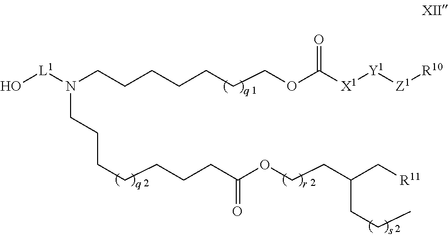

or a pharmaceutically acceptable salt or solvate thereof,
wherein
q¹ is 0, 1, 2, or 3;
q² is 0, 1, 2, or 3;
r² is 0, 1, or 2;
s is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I or below.

In certain embodiments, the compound is a compound of Formula XII″, wherein Z¹ is an optionally substituted C₅-C₁₂ bridged cycloalkylenyl.

In another embodiment, the disclosure provides a compound of Formula XII′″:

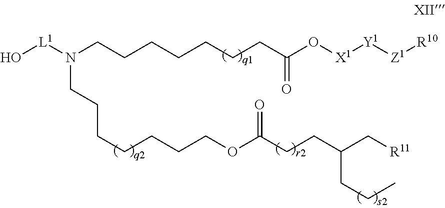

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I or below.

In certain embodiments, the compound is a compound of Formula XII′″, wherein Z¹ is an optionally substituted C₅-C₁₂ bridged cycloalkylenyl.

In another embodiment, the disclosure provides a compound of Formula XIII:

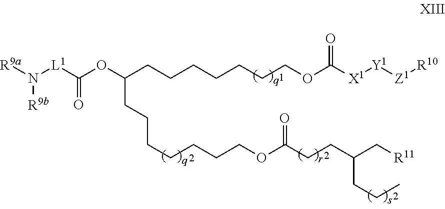

or a pharmaceutically acceptable salt or solvate thereof,
wherein
q¹ is 0, 1, 2, or 3:
q² is 0, 1, 2, or 3:
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula I or below.

In certain embodiments, the compound is a compound of Formula XIII, wherein Z¹ is an optionally substituted C₅-C₁₂ bridged cycloalkylenyl.

In another embodiment, the disclosure provides a compound of Formula XIII′:

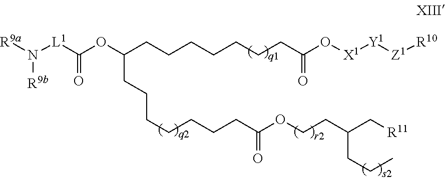

or a pharmaceutically acceptable salt or solvate thereof,
wherein
q¹ is 0, 1, 2, or 3;
q² is 0, 1, 2, or 3;
r² is 0, 1,or 2;
s² is 0, 1,2, 3,4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In certain embodiments, the compound is a compound of Formula XIII′, wherein Z¹ is an optionally substituted C₅-C₁₂ bridged cycloalkylenyl.

In another embodiment, the disclosure provides a compound of Formula XIII″:

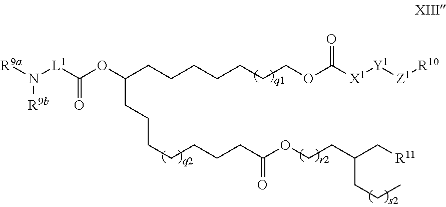

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In certain embodiments, the compound is a compound of Formula XIII″, wherein Z¹ is an optionally substituted C₅-C₁₂ bridged cycloalkylenyl.

In another embodiment, the disclosure provides a compound of Formula XIII′″:

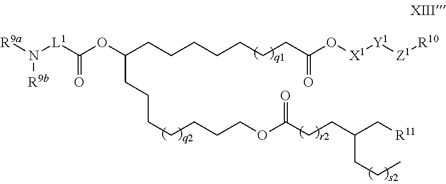

or a pharmaceutically acceptable salt or solvate thereof,
wherein
q¹ is 0, 1, 2, or 3;
q² is 0, 1, 2, or 3;
r² is 0, l, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I or below.

In certain embodiments, the compound is a compound of Formula XIII′″, wherein Z¹ is an optionally substituted C₅-C₁₂ bridged cycloalkylenyl.

In another embodiment, the disclosure provides a compound of Formula XIV:

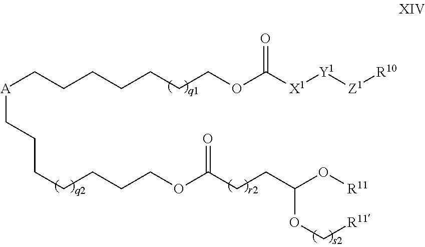

or a pharmaceutically acceptable sat or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r is 0, 1, or 2;
s is 0, 1, 2, 3, 4, 5, 6; and
A, X¹, Y¹, Z¹, R¹⁰ and R¹¹ are as defined herein in Formula IA, Formula IB, Formula IC, Formula ID, Formula I, or below.

In certain embodiments, the compound is a compound of Formula XIV, wherein Z¹ is an optionally substituted C₅-C₁₂ bridged cycloalkylenyl. In certain embodiments, Z¹ is not adamantyl.

In another embodiment, the disclosure provides a compound of Formula XIV′:

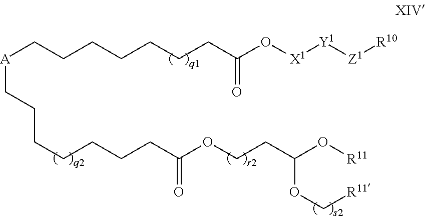

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XIV′, wherein Z¹ is an optionally substituted C₅-C₁₂ bridged cycloalkylenyl. In certain embodiments, Z¹ is not adamantyl.

In another embodiment, the disclosure provides a compound of Formula XIV″:

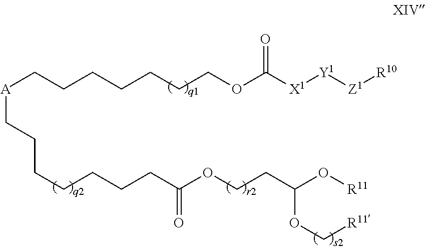

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I or below.

In certain embodiments, the compound is a compound of Formula XIV″, wherein Z¹ is an optionally substituted C₅-C₁₂ bridged cycloalkylenyl. In certain embodiments, Z¹ is not adamantyl.

In another embodiment, the disclosure provides a compound of Formula XIV′″:

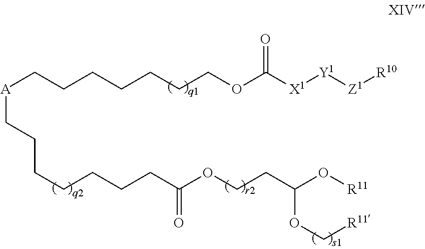

or a pharmaceutically acceptable salt or solvate thereof,
wherein
R^{11′} is selected from the group consisting of hydrogen, C₁-C₁₀ alkyl, and C₁-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I or below.

In certain embodiments, the compound is a compound of Formula XIV′″, wherein Z¹ is an optionally substituted C₅-C₁₂ bridged cycloalkylenyl. In certain embodiments, Z¹ is not adamantyl.

In another embodiment, the disclosure provides a compound of Formula XV:

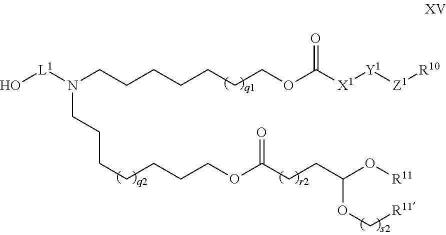

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV′:

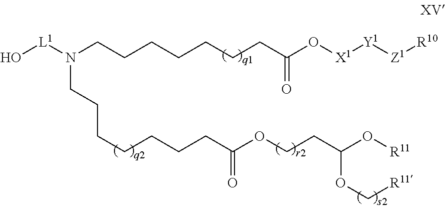

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3:
r² is 0, 1, or 2;
s² is 0, 1, 2, 3, 4, 5, 6; and
L¹, X¹, Y¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV″:

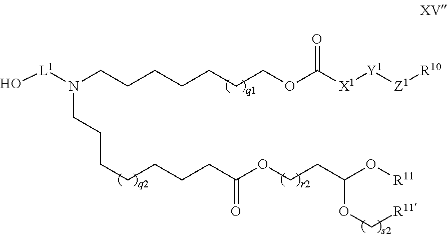

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3:
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV′″:

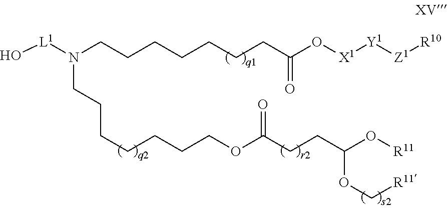

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3:
q² is 0, 1, 2, or 3:
r² is 0, 1,or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XVI:

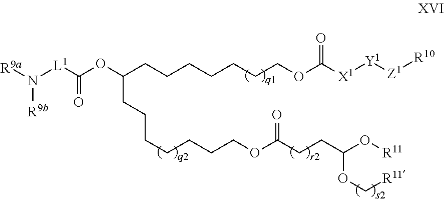

or a pharmaceutically acceptable salt or solvate thereof
wherein
R^{11′} is selected from the group consisting of hydrogen, C₁-C₁₀ alkyl, and C₂-C₁₀ alkenyl;
q is 0, 1, 2, or 3;
q¹ is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI′:

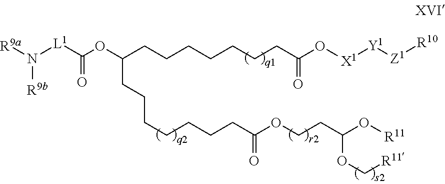

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI″:

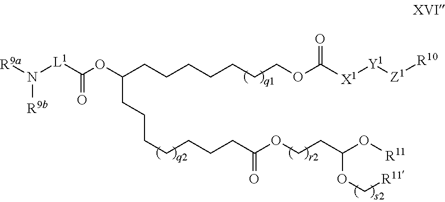

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI′″:

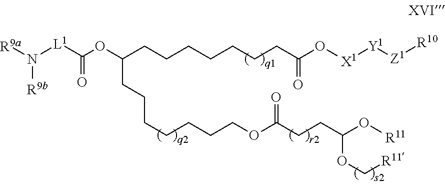

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVII:

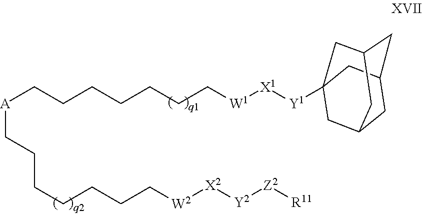

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula XVII, wherein one or more methylene linkages of X², Y², Z², and R¹¹, are not replaced with a group selected from —O—, —CH═CH—, —S— and C₃-C₆ cycloalkylenyl.

In another embodiment, the disclosure provides a compound of Formula XVIII:

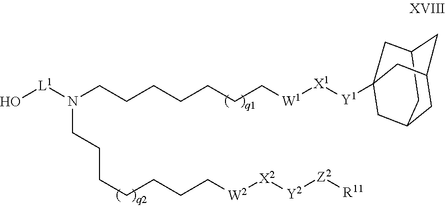

or a pharmaceutically acceptable salt or solvate thereof,
wherein
q¹ is 0, 1, 2, or 3;
q² is 0, 1, 2, or 3;
L¹, X¹, X², Y¹, Y², Z², an R are as defined herein in Formula IA, Formula IB, Formula IC, Formula ID, Formula I, or below.

In certain embodiments, the compound is a compound of Formula XVIII, wherein one or more methylene linkages of X², Y², Z², and R¹¹, are not replaced with a group selected from —O—, —CH═CH—, —S— and C₃-C₆ cycloalkylenyl.

In another embodiment, the disclosure provides a compound of Formula XVIII′:

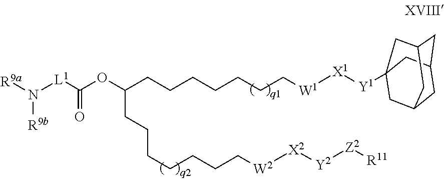

or a pharmaceutically acceptable salt or solvate thereof,
wherein
q¹ is 0, 1, 2, or 3:
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula XIX:

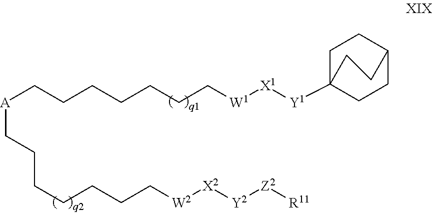

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula XX:

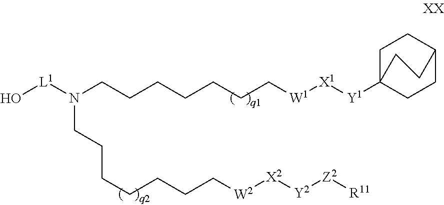

or a pharmaceutically acceptable salt or solvate thereof,
wherein
q¹ is 0, 1, 2, or 3;
q² is 0, 1, 2, or 3;
L¹, X¹, X², Y¹, Y², Z², an R¹¹ are as defined herein in Formula IA, Formula IB, Formula IC, Formula ID, Formula I, or below.

In another embodiment, the disclosure provides a compound of Formula XXI:

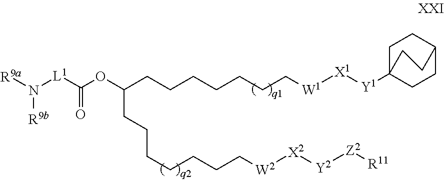

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

### L¹

In another embodiment, L¹ is selected from the group consisting of —CH₂CH₂—, —CH₂CH₂CH₂—, and —CH₂CH₂CH₂CH₂—. In another embodiment, L¹ is —CH₂CH₂—. In another embodiment, L¹ is —CH₂CH₂CH₂—. In another embodiment, L¹ is —CH₂CH₂CH₂CH₂—. In certain embodiments, L¹ is —(CH₂)₂₋₆—OC(═O)—. In some embodiments, L¹ is —(CH₂)₂— OC(═O)—.

### R¹

In another embodiment, R¹ is

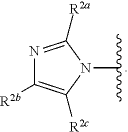

In some embodiments, R¹ is

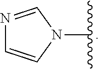 [SMILES: *n1ccnc1]

In another embodiment, R^{2a}, R^{2b}, and R^{2c} are independently selected from the group consisting of hydrogen and methyl. In another embodiment, R^{2a}, R^{2b}, and R^{2c} are independently hydrogen. In another embodiment, R^{2a}, R^{2b}, and R^{2c} are independently methyl.

In another embodiment, R¹ is

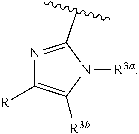

In another embodiment, R^{3a}, R^{3b}, and R^{3c} are independently selected from the group consisting of hydrogen and methyl. In another embodiment, R^{3a}, R^{3b}, and R^{3c} are independently hydrogen. In another embodiment, R³, R^{3b}, and R^{3c} are independently methyl.

In another embodiment, R¹ is

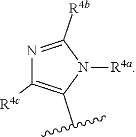

In another embodiment, R^{4a}, R^{4b}, and R^{4c} are independently selected from the group consisting of hydrogen and methyl. In another embodiment, R^{4a}, R^{4b}, and R^{4c} are independently hydrogen. In another embodiment, R^{4a}, R^{4b}, and R^{4c} are independently methyl.

In another embodiment, R¹ is

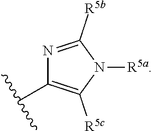

In another embodiment, R^{5a}, R^{5b}, and R^{5c} are independently selected from the group consisting of hydrogen and methyl. In another embodiment, R^{5a}, R^{5b}, and R^{5c} are independently hydrogen. In another embodiment, R^{5a}, R^{5b}, and R^{5c} are independently methyl.

In another embodiment, R¹ is

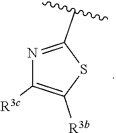

In some embodiments, R¹ is

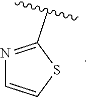 [SMILES: *c1nccs1]

In another embodiment, R^{3b}, and R^{3c} are independently selected from the group consisting of hydrogen and methyl. In another embodiment, R^{3b}, and R^{3c} are independently hydrogen. In another embodiment, R^{3b}, and R^{3c} are independently methyl.

In another embodiment, R¹ is

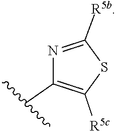

In some embodiments, R¹ is

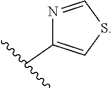 [SMILES: *c1cscn1]

In another embodiment, R^{5b}, and R^{5c} are independently selected from the group consisting of hydrogen and methyl. In another embodiment, R^{5b}, and R^{5c} are independently hydrogen. In another embodiment, R^{5b}, and R^{5c} are independently methyl.

In another embodiment, R¹ is —OH.

In some embodiments, R¹ is —N(R^{9a})(R^{9b}). In some embodiments, R¹ is —NMe₂.

In some embodiments, R¹ is —NEt₂.

In another embodiment, R¹ is

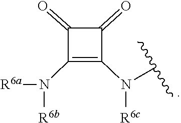

In another embodiment, R¹ is

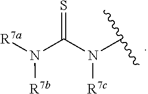

### L²

In another embodiment, L² is selected from the group consisting of —CH₂CH₂—, —CH₂CH₂CH₂—, and —CH₂CH₂CH₂CH₂—. In another embodiment, L² is —CH₂CH₂—. In another embodiment, L² is —CH₂CH₂CH₂—. In another embodiment, L² is —CH₂CH₂CH₂CH₂—.

### R⁸

In another embodiment, R⁸ is

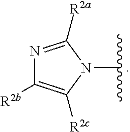

In some embodiments, R⁸ is

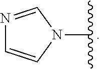 [SMILES: *n1ccnc1]

In another embodiment, R^{2a}, R^{2b}, and R^{2c} are independently selected from the group consisting of hydrogen and methyl. In another embodiment, R^{2a}, R^{2b}, and R^{2c} are independently hydrogen. In another embodiment, R^{2a}, R^{2b}, and R^{2c} are independently methyl.

In another embodiment, R⁸ is

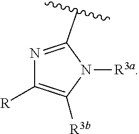

In another embodiment, R^{3a}, R^{3b}, and R^{3c} are independently selected from the group consisting of hydrogen and methyl. In another embodiment, R^{3a}, R^{3b}, and R^{3c} are independently hydrogen. In another embodiment, R^{3a}, R^{3b}, and R^{3c} are independently methyl.

In another embodiment, R⁸ is

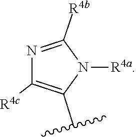

In another embodiment, R^{4a}, R^{4b}, and R^{4c} are independently selected from the group consisting of hydrogen and methyl. In another embodiment, R^{4a}, R^{4b}, and R^{4c} are independently hydrogen. In another embodiment, R^{4a}, R^{4b}, and R^{4c} are independently methyl.

In another embodiment, R⁸ is

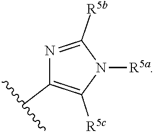

In another embodiment, R^{a}, R^{5b}, and R^{5c} are independently selected from the group consisting of hydrogen and methyl. In another embodiment, R^{5a}, R^{5b}, and R^{5c} are independently hydrogen. In another embodiment, R^{5a}, R^{5b}, and R^{5c} are independently methyl.

In another embodiment, R⁸ is

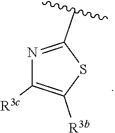

In some embodiments, R⁸ is

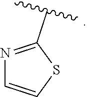 [SMILES: *c1nccs1]

In another embodiment, R^{3b}, and R^{3c} are independently selected from the group consisting of hydrogen and methyl. In another embodiment, R^{3b}, and R^{3c} are independently hydrogen. In another embodiment, R^{3b}, and R^{3c} are independently methyl.

In another embodiment, R⁸ is

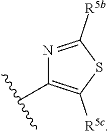

In some embodiments, R⁸ is

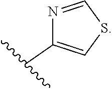 [SMILES: *c1cscn1]

In another embodiment, R^{5b}, and R^{5c} are independently selected from the group consisting of hydrogen and methyl. In another embodiment, R^{5b}, and R^{5c} are independently hydrogen. In another embodiment, R^{5b}, and R^{5c} are independently methyl.

In another embodiment, R⁸ is —NR^{9a}R^{9b}. In some embodiments, R⁸ is —NMe₂. In some embodiments, R⁸ is —NEt₂.

In another embodiment, R⁸ is —OH.

R^{9a}, R^{9b}

In another embodiment, R^{9a} and R^{9b} are independently selected from the group consisting of hydrogen and C₁-C₄ alkyl. In another embodiment, R^{9a} and R^{9b} are each methyl. In another embodiment, R^{9a} and R^{9b} are each ethyl.

### R′

In another embodiment, R′ is hydrogen. In some embodiments, R′ is C₁-C₆ alkyl.

### Q¹

In another embodiment, Q¹ is straight chain C₁-C₂₀ alkylenyl. In another embodiment, Q¹ is straight chain C₁-C₁₀ alkylenyl. In another embodiment, Q¹ is C₁-C₁₀ alkylenyl. In another embodiment, Q¹ is C₂-C₅ alkylenyl. Q¹ is C₆-C₉ alkylenyl. In another embodiment, Q¹ is selected from the group consisting of —CH₂CH₂—, —CH₂CH₂CH₂— —CH₂(CH₂)₂CH₂—, —CH₂(CH₂)₃CH₂—, —CH₂(CH₂)₄CH₂—, —CH₂(CH₂)₅CH₂—, —CH₂(CH₂)₆CH₂— —CH₂(CH₂)₇CH₂—, and —CH₂(CH₂)₈CH₂—. In another embodiment, Q¹ is —CH₂CH₂—. In another embodiment, Q¹ is —CH₂CH₂CH₂—. In another embodiment, Q¹ is —CH₂(CH₂)₂CH₂—. In another embodiment, Q¹ is —CH₂(CH₂)₃CH₂—. In another embodiment, Q¹ is —CH₂CH₂—. In another embodiment, Q¹ is —CH₂(CH₂)₄CH₂—. In another embodiment, Q¹ is —CH₂(CH₂)₅CH₂—. In another embodiment, Q¹ is —CH₂(CH₂)₆CH₂—. In another embodiment, Q¹ is —CH₂(CH₂)₇CH₂—.

In another embodiment, Q¹ is —CH₂(CH₂)₈·CH₂—.

### W¹

In another embodiment, W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—. In another embodiment, W¹ is —C(═O)O—. In another embodiment, W¹ is —OC(═O)—. In another embodiment, W¹ is —C(═O)N(R^{12a})—. In another embodiment, W¹ is —N(R^{12a})C(═O)—. In another embodiment, W¹ is —OC(═O)N(R^{12a})—. In another embodiment, W¹ is —N(R^{12a})C(═O)O—. In another embodiment, W¹ is —OC(═O)O—.

### X¹

In another embodiment, X² is optionally substituted C₁-C₁₅ alkylenyl. In another embodiment, X² is branched C₁-C₁₅ alkylenyl. In another embodiment, X¹ is a bond or C₁-C₁₅ alkylenyl. In another embodiment, X¹ is a bond. In another embodiment, X¹ is C₂-C₅ alkylenyl. In another embodiment, X¹ is C₆-C₉ alkylenyl. In another embodiment, X¹ is —CH₂—. In another embodiment, X² is —CH₂CH₂—. In another embodiment, X² is —CH₂CH₂CH₂—. In another embodiment, X² is —CH₂CH₂CH₂CH₂—. In another embodiment, X² is —CH₂CH₂CH₂CH₂CH₂—.

### Y¹

In another embodiment, Y¹ is selected from the group consisting of —(CH₂)ₘ—, —O—, —S—, and —S—S—. In another embodiment, Y¹ is —(CH₂)ₘ—. In some embodiments, Y¹ is —O—. In some embodiments, Y¹ is —S—. In another embodiment, Y¹ is —CH₂—. In another embodiment, Y² is —CH₂CH₂—.

m

In another embodiment, m is 0. In another embodiment, m is 1. In another embodiment, m is 2. In another embodiment, m is 3. In another embodiment, m is 4. In another embodiment, m is 5. In another embodiment, m is 6.

n

In another embodiment, n is 0. In another embodiment, n is 1. In another embodiment, n is 2. In another embodiment, n is 3. In another embodiment, n is 4. In another embodiment, n is 5. In another embodiment, n is 6.

p

In another embodiment, p is 0. In another embodiment, p is 1.

### Z¹

In another embodiment, Z¹ is selected from the group consisting of C₄-C₁₂ cycloalkylenyl,

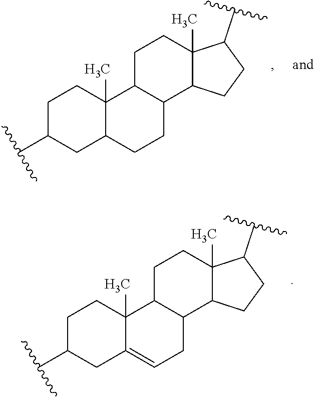 [SMILES: *C1CCC2(C)C(CCC3C4CCC(*)C4(C)CCC32)C1 ; *C1CCC2(C)C(=CCC3C2CCC2(C)C(*)CCC32)C1]

In certain embodiments, Z¹ is optionally substituted.

In another embodiment, Z¹ is

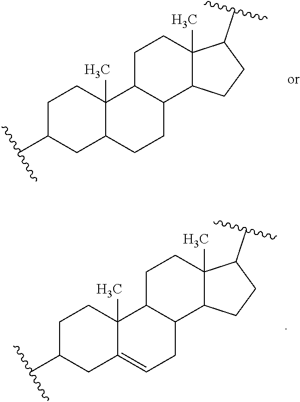 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, Z¹ is C₄-C₁₂ cycloalkylenyl. In another embodiment, Z¹ is a monocyclic C₄-C₈ cycloalkylenyl. In another embodiment, Z¹ is a monocyclic C₄-C₆ cycloalkylenyl. In another embodiment, Z¹ is a monocyclic C₄ cycloalkylenyl. In another embodiment, Z¹ is a monocyclic C₅ cycloalkylenyl. In another embodiment, Z¹ is a monocyclic C₆ cycloalkylenyl.

In another embodiment, Z¹ is an optionally substituted bridged bicyclic or multicyclic cycloalkylenyl. In some embodiments, Z¹ is optionally substituted C₅-C₁₂ bridged cycloalkylenyl. In some embodiments, Z¹ is optionally substituted C₆-C₁₀ bridged cycloalkylenyl. In some embodiments, Z¹ is a optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, Z¹ is selected from the group consisting of:

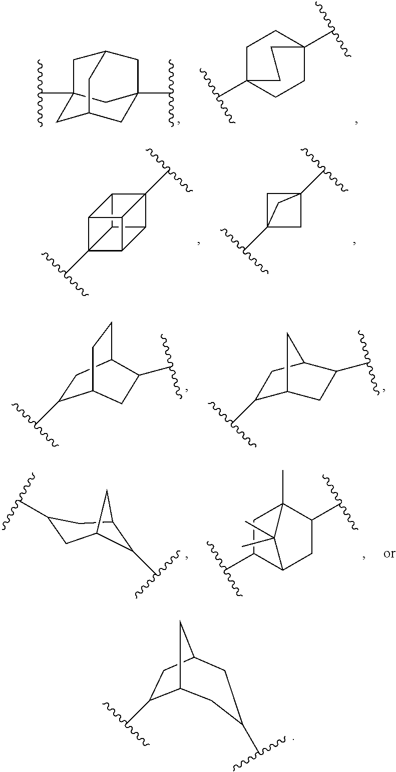

In another embodiment, Z¹ is selected from the group consisting of:

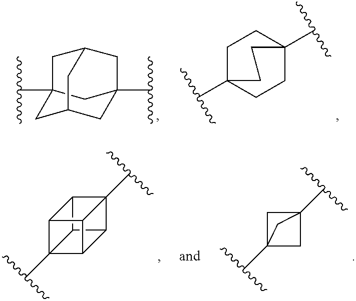 [SMILES: *C12CC3CC(C1)CC(*)(C3)C2 ; *C12CCC(*)(CC1)CC2 ; *C12C3C4C1C1C2C3C41* ; *C12CC(*)(C1)C2]

In some embodiments, Z¹ is adamantyl. In another embodiment, Z¹ is

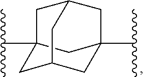 [SMILES: *C12CC3CC(C1)CC(*)(C3)C2]

In some embodiments, Z¹ is bicyclo[2.2.2]octyl. In another embodiment, Z¹ is

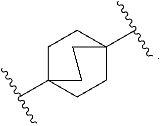 [SMILES: *C12CCC(*)(CC1)CC2]

In some embodiments, Z¹ is cubanyl. In another embodiment, Z¹ is

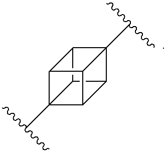 [SMILES: *C12C3C4C1C1C2C3C41*]

In some embodiments, Z¹ is bicyclo[1.1.1]pentyl. In another embodiment, Z¹

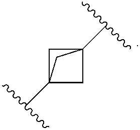 [SMILES: *C12CC(*)(C1)C2]

is. In some embodiments, Z¹ is bicyclo[2.2.1]heptyl. In another embodiment, Z¹ is

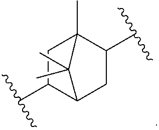 [SMILES: *C1CC2(C)C(*)CC1C2(C)C]

In another embodiment, Z¹ is selected from the group consisting of:

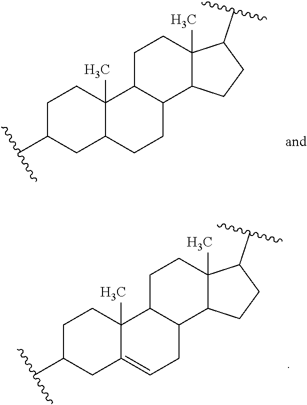 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, Z¹ is

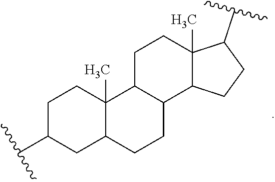 [SMILES: *C1CCC2(C)C(CCC3C4CCC(*)C4(C)CCC32)C1]

In another embodiment, Z¹ is

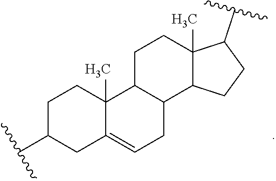 [SMILES: *C1CCC2(C)C(=CCC3C2CCC2(C)C(*)CCC32)C1]

### R¹⁰

In another embodiment, R¹⁰ is hydrogen.

In another embodiment, R¹⁰ is C₁-C₁₀ alkyl. In another embodiment, R¹⁰ is C₃-C₇ alkyl. In another embodiment, R¹⁰ is C₄-C₆ alkyl. In another embodiment, R¹⁰ is C₄. In another embodiment, R¹⁰ is Cs. In another embodiment, R¹⁰ is C₆.

In another embodiment, R¹⁰ is C₂-C₁₂ alkenyl. In another embodiment, R¹⁰ is C₆-C₁₂ alkenyl. In another embodiment, R¹⁰ is C₂-C₈ alkenyl.

### R¹¹

In another embodiment, R¹ is C₁-C₁₀ alkyl. In another embodiment, R¹¹ is optionally substituted C₁-C₂₀ alkyl. In another embodiment, R¹¹ is optionally substituted branched C₁-C₂₀ alkyl. In another embodiment, R¹¹ is optionally substituted C₁-C₁₅ alkyl. In another embodiment, R¹¹ is optionally substituted C₁-C₁₅ branched alkyl. In another embodiment, R¹¹ is optionally substituted C₁₀-C₁₅ alkyl. In another embodiment, R¹¹ is optionally substituted C₁₀-C₁₅ branched alkyl. In another embodiment, R^{1a} is selected from the group consisting of —CH₃, —CH₂CH₃, and —CH₂CH₂CH₃. In another embodiment, R¹¹ is selected from the group consisting of —CH₂(CH₂)₂CH₃, —CH₂(CH₂)₃CH₃, —CH₂(CH₂)₄CH₃, —CH₂(CH₂)₈CH₃, —CH₂(CH₂)₆CH₃, —CH₂(CH₂)₇CH₃, and —CH₂(CH₂)₈CH₃. In another embodiment, R¹¹ is —CH₃. In another embodiment, R¹¹ is —CH₂CH₃. In another embodiment, R¹¹ is —CH₂CH₂CH₃. In another embodiment, R¹¹ is —CH₂(CH₂)₂CH₃. In another embodiment, R¹¹ is —CH₂(CH₂)₃CH₃. In another embodiment, R¹¹ is —CH₂(CH₂)₄CH₃. In another embodiment, R¹¹ is —CH₂(CH₂)₅CH₃. In another embodiment, R¹¹ is CH₂(CH₂)₆CH₃. In another embodiment, R¹¹ is —CH₂(CH₂)₇CH₃. In another embodiment, R¹¹ is —CH₂(CH₂)₈CH₃.

In another embodiment, R¹¹ is C₂-C₁₀ alkenyl. In another embodiment, R¹¹ is C₂-C₁₂ alkenyl. In another embodiment, R¹¹ is C₆-C₁₂ alkenyl. In another embodiment, R¹¹ is C₂-C₈ alkenyl.

In another embodiment, the disclosure provides a compound of any one of Formulae IA, IB, IC, or I-XXI or a pharmaceutically acceptable salt or solvate thereof, wherein R¹¹ is hydrogen.

### Q²

In another embodiment, Q² is straight chain C₁-C₂₀ alkylenyl. In another embodiment, Q² is straight chain C₁-C₁₀ alkylenyl. In another embodiment, Q² is C₂-C₁O alkylenyl. In another embodiment, Q² is selected from the group consisting of —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, —CH₂(CH₂)₆CH₂—, —CH₂(CH₂)₇CH₂—, and —CH₂(CH₂)₈·CH₂—. In another embodiment, Q² is —CH₂CH₂—. In another embodiment, Q² is —CH₂CH₂CH₂—. In another embodiment, Q² is —CH₂(CH₂)₃CH₂—. In another embodiment, Q² is —CH₂(CH₂)₄CH₂—. In another embodiment, Q² is —CH₂(CH₂)₅CH₂—. In another embodiment, Q² is —CH₂(CH₂)₆CH₂—. In another embodiment, Q² is —CH₂(CH₂)₇CH₂—. In another embodiment, Q² is —CH₂(CH₂)₈·CH₂—.

### W²

In another embodiment, W² is selected from the group consisting of —C(═O)O— and —OC(═O)—. In another embodiment, W² is —C(═O)O—. In another embodiment, W² is —OC(═O)—.

### X²

In another embodiment, X² is optionally substituted C₁-C₁₅ alkylenyl. In another embodiment, X² is C₁-C₁₅ branched alkylenyl. In another embodiment, X² is C₁-C₆ alkylenyl or a bond. In another embodiment, X² is C₂-C₄ alkylenyl. In another embodiment, X² is C₃-C₅ alkylenyl. In another embodiment, X² is selected from the group consisting of —CH₂CH₂—, —CH₂CH₂CH₂—, —CH₂(CH₂)₂CH₂—, —CH₂(CH₂)₃CH₂—, and —CH₂(CH₂)₄CH₂—. In another embodiment, X² is —CH₂—. In another embodiment, X² is a bond. In another embodiment, X² is branched C₁-C₁₅ alkylenyl, wherein one or more methylene linkages of X² are optionally and independently replaced with a group selected from —O—, —CH═CH—, —S— and C₃-C₆ cycloalkylenyl.

### Y²

In another embodiment, Y² is selected from the group consisting of —(CH₂)ₘ— and —S—. In another embodiment, Y² is —(CH₂)ₘ—. In another embodiment, Y² is —S—.

### Z²

In another embodiment, Z² is —(CH₂)ₚ—. In another embodiment, Z² is —CH₂—. In another embodiment, Z² is —CH₂CH₂—. In another embodiment, Z² is C₄-C₁₂ cycloalkylenyl. In another embodiment, Z² is a monocyclic C₄-C₈ cycloalkylenyl. In certain embodiments, Z² is optionally substituted.

In another embodiment, Z² is an optionally substituted bridged bicyclic or multicyclic cycloalkylenyl. In some embodiments, Z² is optionally substituted C₅-C₁₂ bridged cycloalkylenyl. In some embodiments, Z² is optionally substituted C₆-C₁₀ bridged cycloalkylenyl. In some embodiments, Z² is a optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, Z² is selected from the group consisting of:

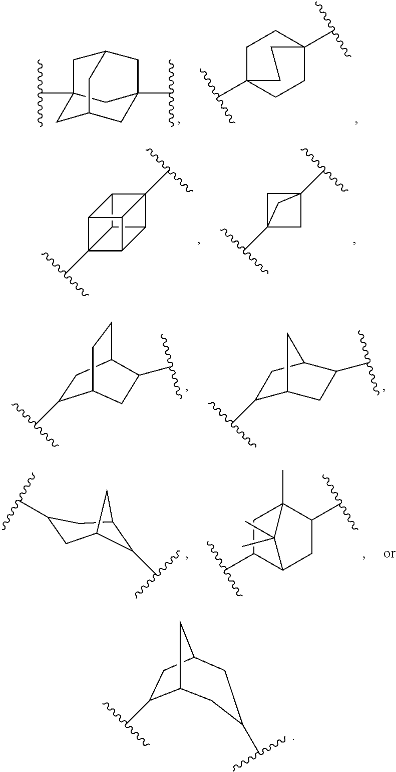

In another embodiment, Z² is

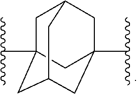 [SMILES: *C12CC3CC(C1)CC(*)(C3)C2]

In another embodiment, Z² is

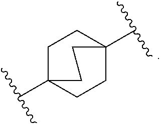 [SMILES: *C12CCC(*)(CC1)CC2]

In another embodiment, Z² is

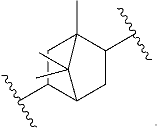 [SMILES: *C1CC2(C)C(*)CC1C2(C)C]

In another embodiment, Z² is selected from the group consisting of:

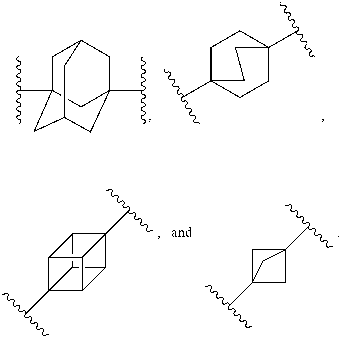

In another embodiment, Z² is selected from the group consisting of:

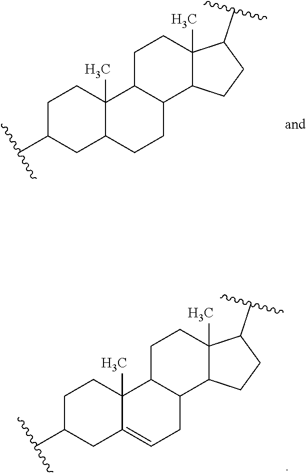 [SMILES: *C1CCC2(C)C(CCC3C4CCC(*)C4(C)CCC32)C1 ; *C1CCC2(C)C(=CCC3C2CCC2(C)C(*)CCC32)C1]

In some embodiments, -Q¹-W¹—X¹—Y¹—Z¹—R¹⁰ is selected from the group consisting of:

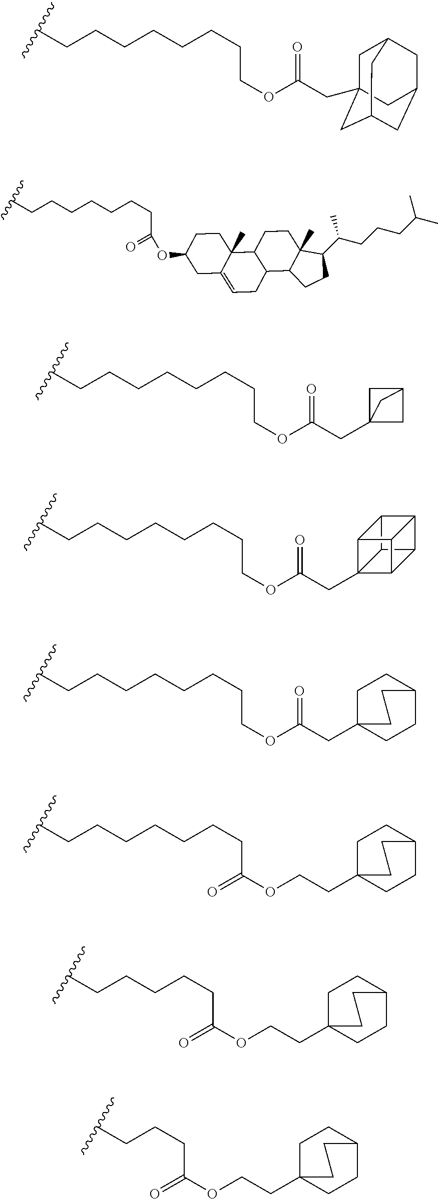

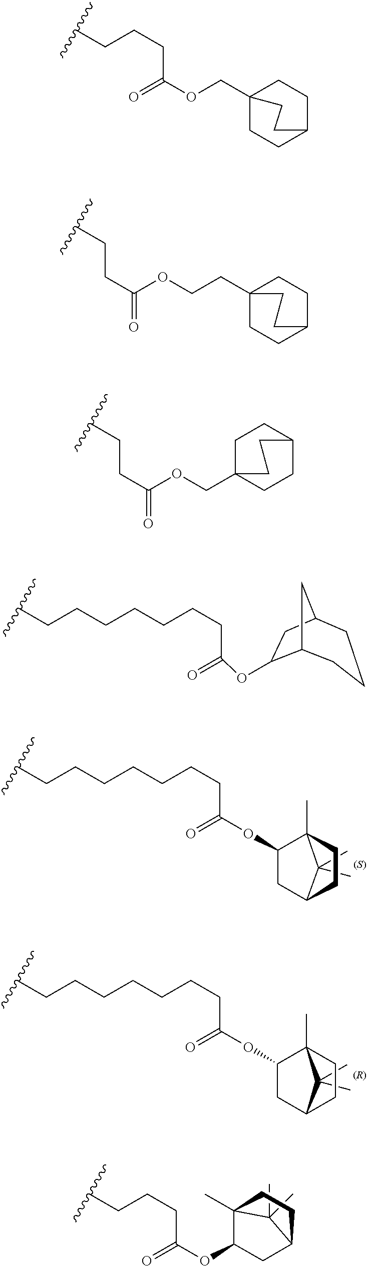

In some embodiments, Q²-W²—X²—Y²—Z²—R¹¹ is selected from the group consisting of:

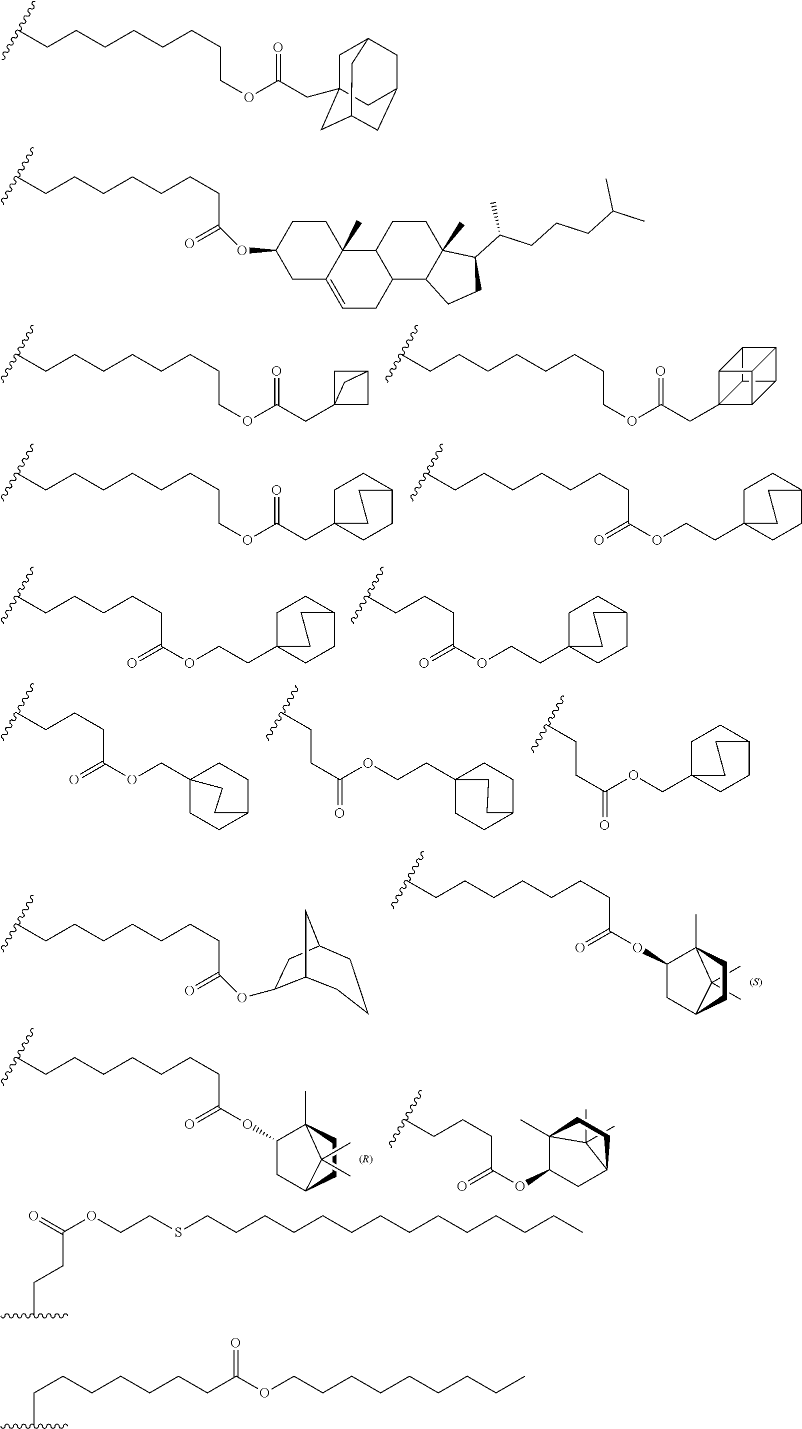

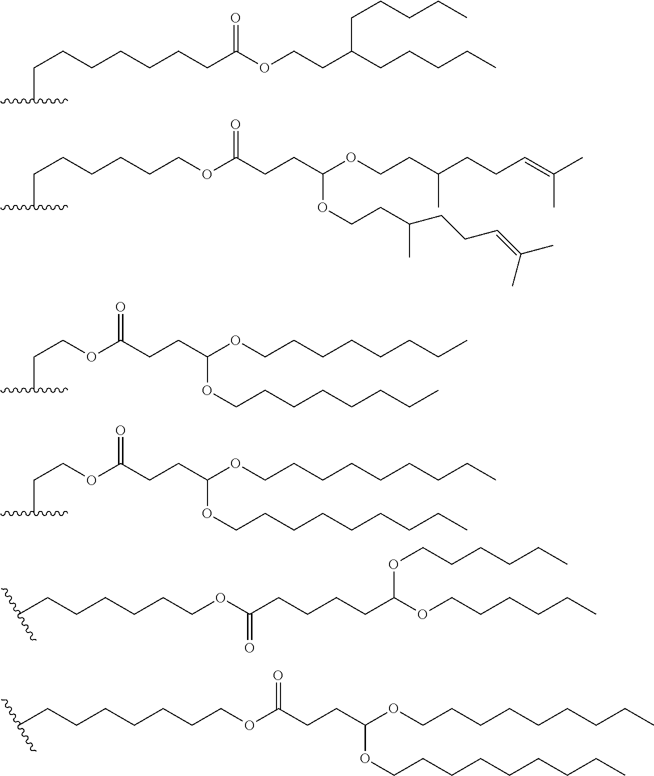

In some embodiments, —W¹—X¹—Y¹—Z¹—R¹⁰ is selected from the group consisting of:

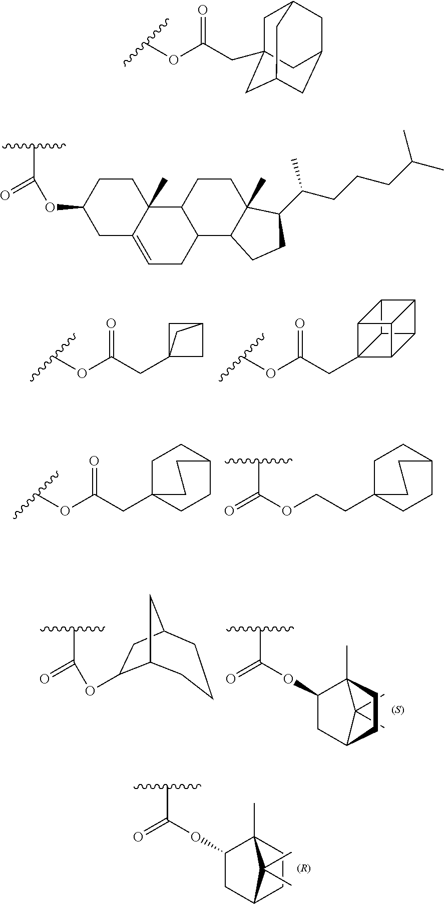

In some embodiments, —W²—X²—Y²—Z²—R¹¹ is selected from the group consisting of:

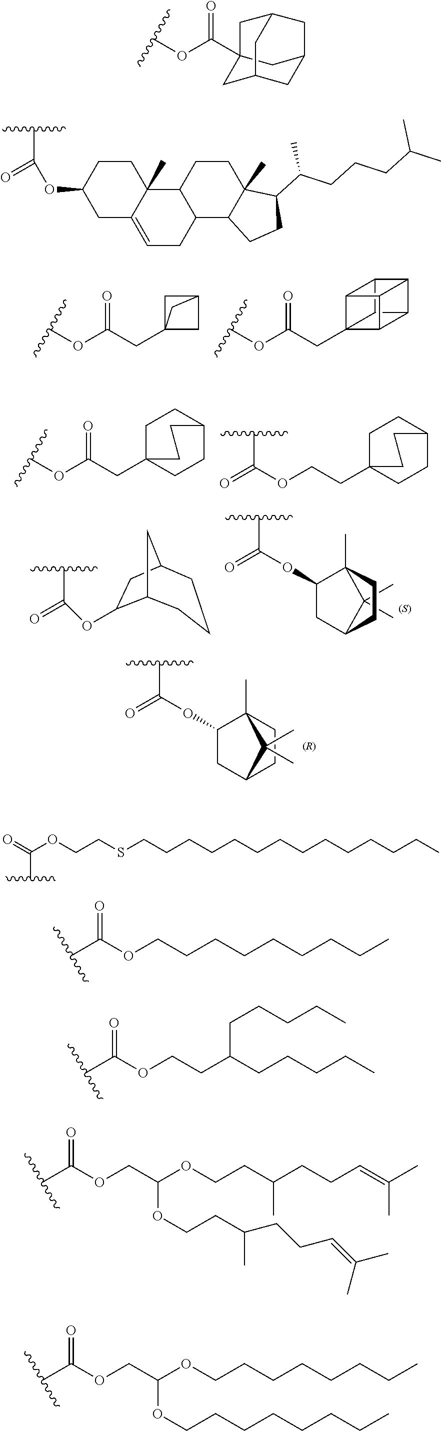

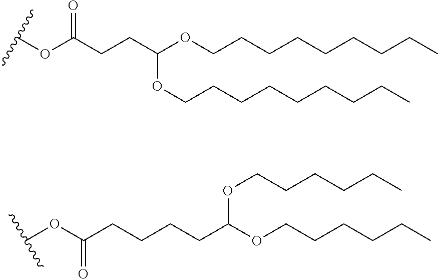 [SMILES: *OC(=O)CCC(OCCCCCCCCC)OCCCCCCCCC ; *OC(=O)CCCCC(OCCCCCC)OCCCCCC]

In another embodiment, the disclosure provides a compound selected from any one of more of the compounds of Table (III), or a pharmaceutically acceptable salt or solvate thereof.

TABLE (III)
Non-Limiting Examples of Ionizable Lipids with a Constrained Arm

| Compound | |
|---|---|
| No. | Structure |
| C1 | 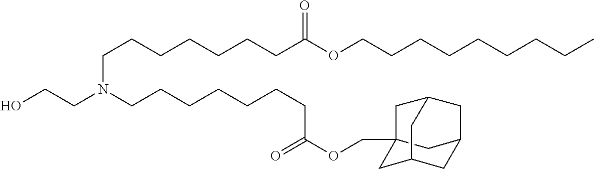 [SMILES: CCCCCCCCCOC(=O)CCCCCCCN(CCO)CCCCCCCC(=O)OCC12CC3CC(CC(C3)C1)C2] |
| C2 | 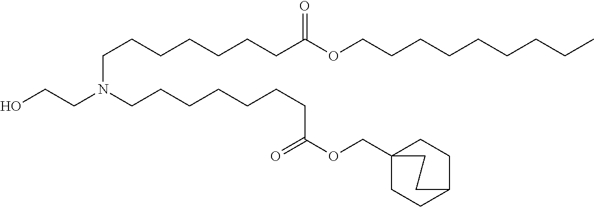 [SMILES: CCCCCCCCCOC(=O)CCCCCCCN(CCO)CCCCCCCC(=O)OCC12CCC(CC1)CC2] |
| C3 | 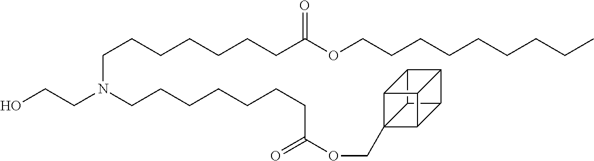 [SMILES: CCCCCCCCCOC(=O)CCCCCCCN(CCO)CCCCCCCC(=O)OCC12C3C4C5C3C1C5C42] |
| C4 | 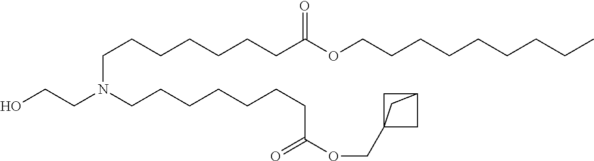 [SMILES: CCCCCCCCCOC(=O)CCCCCCCN(CCO)CCCCCCCC(=O)OCC12CC(C1)C2] |
| C5 | 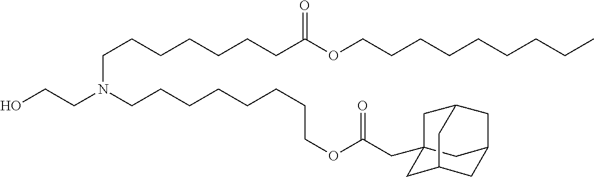 [SMILES: CCCCCCCCCOC(=O)CCCCCCCN(CCO)CCCCCCCCOC(=O)CC12CC3CC(CC(C3)C1)C2] |
| C6 | 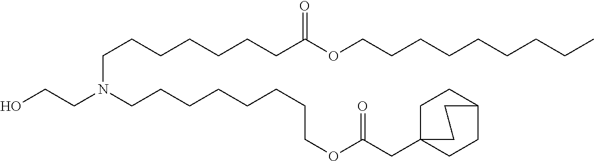 [SMILES: CCCCCCCCCOC(=O)CCCCCCCN(CCO)CCCCCCCCOC(=O)CC12CCC(CC1)CC2] |
| C7 | 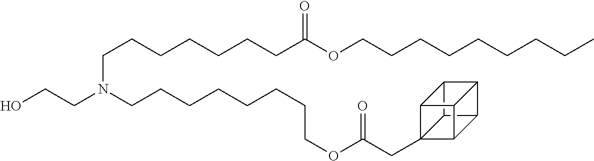 [SMILES: CCCCCCCCCOC(=O)CCCCCCCN(CCO)CCCCCCCCOC(=O)CC12C3C4C5C3C1C5C42] |
| C8 | 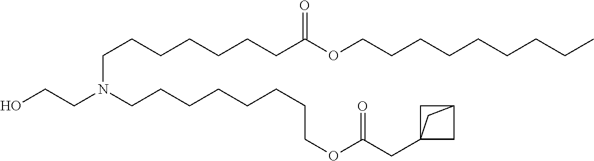 [SMILES: CCCCCCCCCOC(=O)CCCCCCCN(CCO)CCCCCCCCOC(=O)CC12CC(C1)C2] |
| C9 | 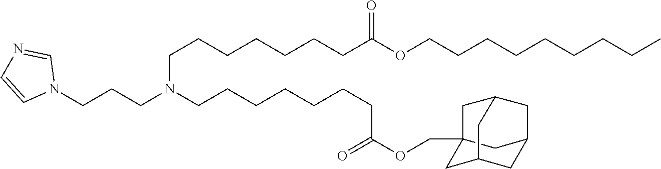 [SMILES: CCCCCCCCCOC(=O)CCCCCCCN(CCCCCCCC(=O)OCC12CC3CC(CC(C3)C1)C2)CCCn1ccnc1] |
| C10 | 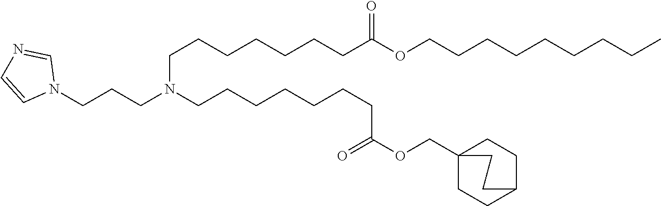 [SMILES: CCCCCCCCCOC(=O)CCCCCCCN(CCCCCCCC(=O)OCC12CCC(CC1)CC2)CCCn1ccnc1] |
| C11 | 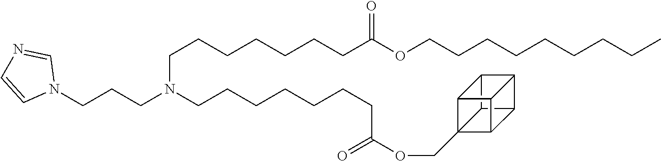 [SMILES: CCCCCCCCCOC(=O)CCCCCCCN(CCCCCCCC(=O)OCC12C3C4C5C3C1C5C42)CCCn1ccnc1] |
| C12 | 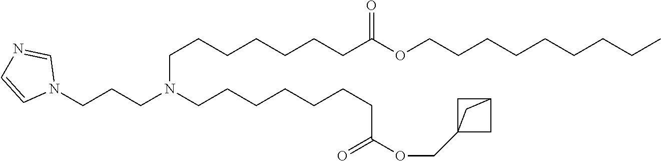 [SMILES: CCCCCCCCCOC(=O)CCCCCCCN(CCCCCCCC(=O)OCC12CC(C1)C2)CCCn1ccnc1] |
| C13 | 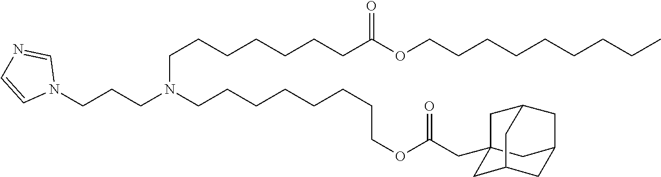 [SMILES: CCCCCCCCCOC(=O)CCCCCCCN(CCCCCCCCOC(=O)CC12CC3CC(CC(C3)C1)C2)CCCn1ccnc1] |
| C14 | 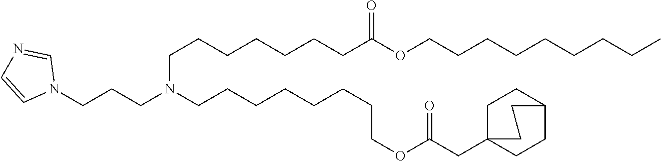 [SMILES: CCCCCCCCCOC(=O)CCCCCCCN(CCCCCCCCOC(=O)CC12CCC(CC1)CC2)CCCn1ccnc1] |
| C15 | 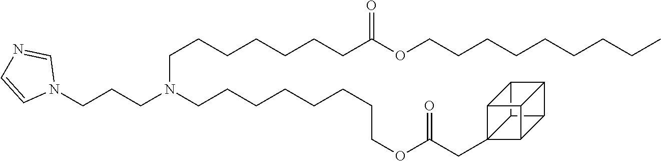 [SMILES: CCCCCCCCCOC(=O)CCCCCCCN(CCCCCCCCOC(=O)CC12C3C4C5C3C1C5C42)CCCn1ccnc1] |
| C16 | 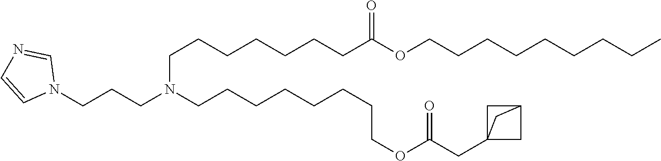 [SMILES: CCCCCCCCCOC(=O)CCCCCCCN(CCCCCCCCOC(=O)CC12CC(C1)C2)CCCn1ccnc1] |
| C17 | 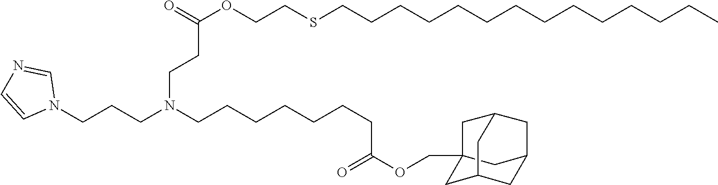 [SMILES: CCCCCCCCCCCCCCSCCOC(=O)CCN(CCCCCCCC(=O)OCC12CC3CC(CC(C3)C1)C2)CCCn1ccnc1] |
| C18 | 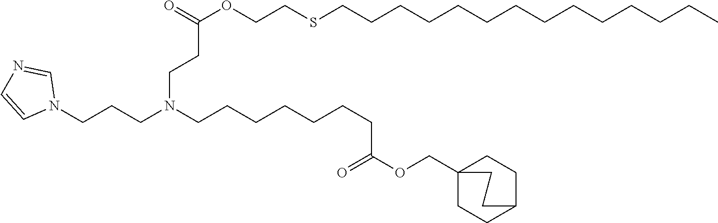 [SMILES: CCCCCCCCCCCCCCSCCOC(=O)CCN(CCCCCCCC(=O)OCC12CCC(CC1)CC2)CCCn1ccnc1] |
| C19 | 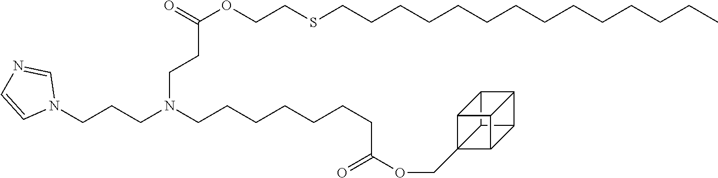 [SMILES: CCCCCCCCCCCCCCSCCOC(=O)CCN(CCCCCCCC(=O)OCC12C3C4C5C3C1C5C42)CCCn1ccnc1] |
| C20 | 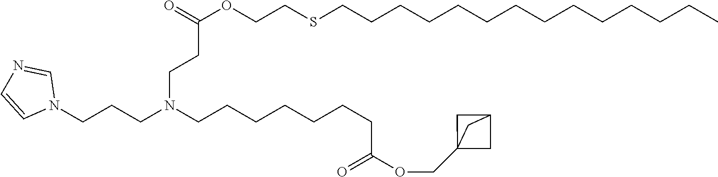 [SMILES: CCCCCCCCCCCCCCSCCOC(=O)CCN(CCCCCCCC(=O)OCC12CC(C1)C2)CCCn1ccnc1] |
| C21 | 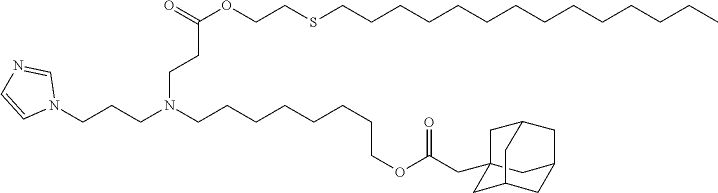 [SMILES: CCCCCCCCCCCCCCSCCOC(=O)CCN(CCCCCCCCOC(=O)CC12CC3CC(CC(C3)C1)C2)CCCn1ccnc1] |
| C22 | 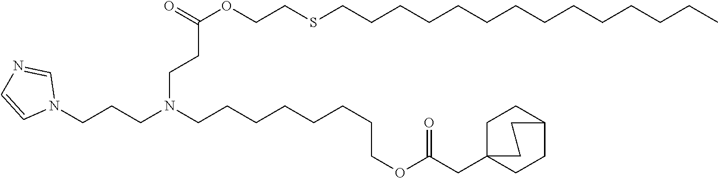 [SMILES: CCCCCCCCCCCCCCSCCOC(=O)CCN(CCCCCCCCOC(=O)CC12CCC(CC1)CC2)CCCn1ccnc1] |
| C23 | 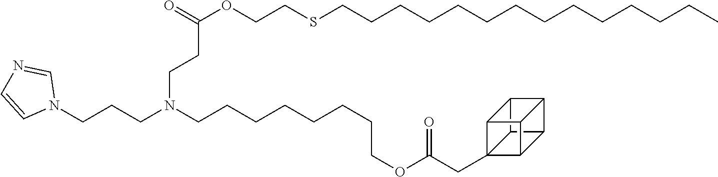 [SMILES: CCCCCCCCCCCCCCSCCOC(=O)CCN(CCCCCCCCOC(=O)CC12C3C4C5C3C1C5C42)CCCn1ccnc1] |
| C24 | 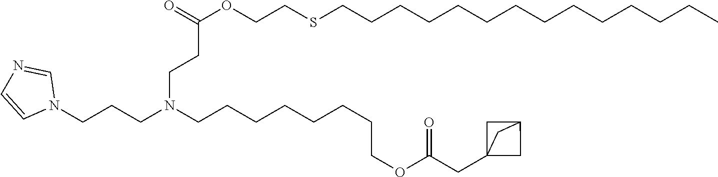 [SMILES: CCCCCCCCCCCCCCSCCOC(=O)CCN(CCCCCCCCOC(=O)CC12CC(C1)C2)CCCn1ccnc1] |
| C25 | 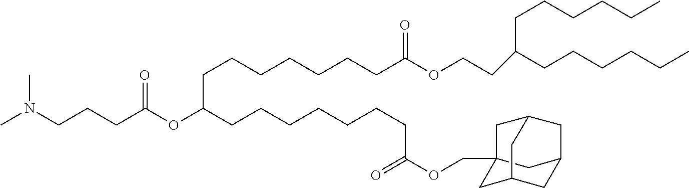 [SMILES: CCCCCCC(CCCCCC)CCOC(=O)CCCCCCCC(CCCCCCCC(=O)OCC12CC3CC(CC(C3)C1)C2)OC(=O)CCCN(C)C] |
| C26 | 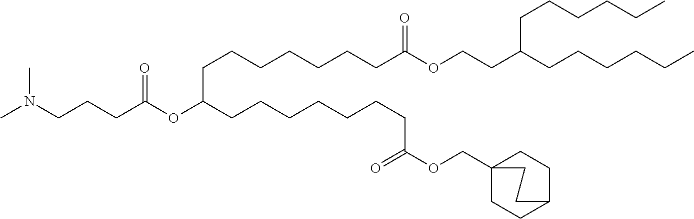 [SMILES: CCCCCCC(CCCCCC)CCOC(=O)CCCCCCCC(CCCCCCCC(=O)OCC12CCC(CC1)CC2)OC(=O)CCCN(C)C] |
| C27 | 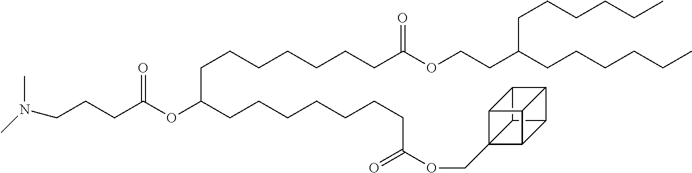 [SMILES: CCCCCCC(CCCCCC)CCOC(=O)CCCCCCCC(CCCCCCCC(=O)OCC12C3C4C5C3C1C5C42)OC(=O)CCCN(C)C] |
| C28 | 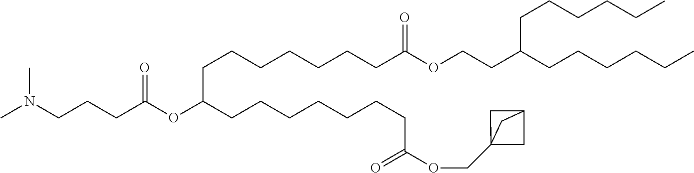 [SMILES: CCCCCCC(CCCCCC)CCOC(=O)CCCCCCCC(CCCCCCCC(=O)OCC12CC(C1)C2)OC(=O)CCCN(C)C] |
| C29 | 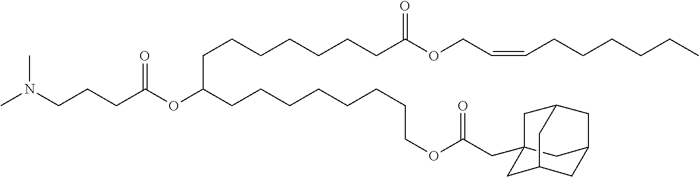 [SMILES: CCCCCC/C=C\COC(=O)CCCCCCCC(CCCCCCCCOC(=O)CC12CC3CC(CC(C3)C1)C2)OC(=O)CCCN(C)C] |
| C30 | 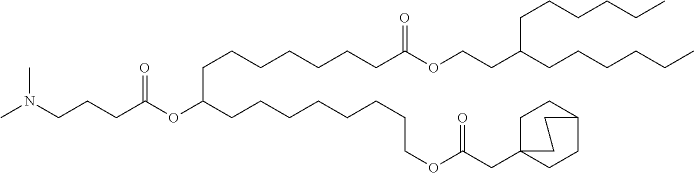 [SMILES: CCCCCCC(CCCCCC)CCOC(=O)CCCCCCCC(CCCCCCCCOC(=O)CC12CCC(CC1)CC2)OC(=O)CCCN(C)C] |
| C31 | 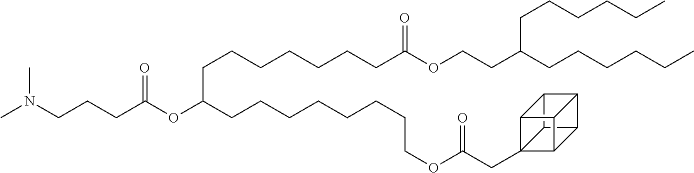 [SMILES: CCCCCCC(CCCCCC)CCOC(=O)CCCCCCCC(CCCCCCCCOC(=O)CC12C3C4C5C3C1C5C42)OC(=O)CCCN(C)C] |
| C32 | 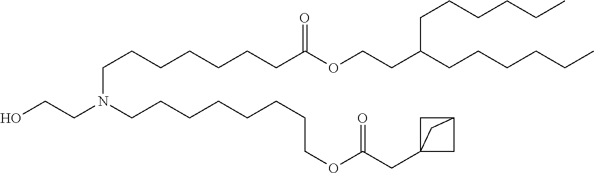 [SMILES: CCCCCCC(CCCCCC)CCOC(=O)CCCCCCCN(CCO)CCCCCCCCOC(=O)CC12CC(C1)C2] |
| C33 | 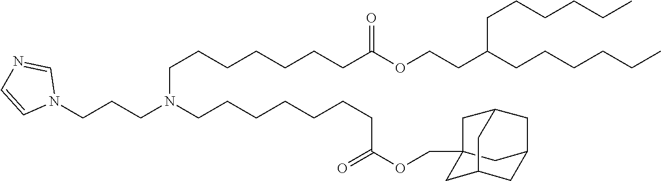 [SMILES: CCCCCCC(CCCCCC)CCOC(=O)CCCCCCCN(CCCCCCCC(=O)OCC12CC3CC(CC(C3)C1)C2)CCCn1ccnc1] |
| C34 | 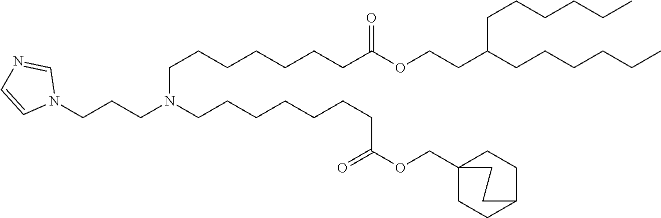 [SMILES: CCCCCCC(CCCCCC)CCOC(=O)CCCCCCCN(CCCCCCCC(=O)OCC12CCC(CC1)CC2)CCCn1ccnc1] |
| C35 | 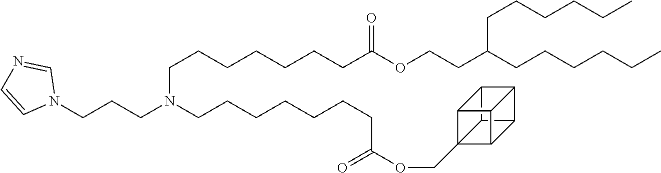 [SMILES: CCCCCCC(CCCCCC)CCOC(=O)CCCCCCCN(CCCCCCCC(=O)OCC12C3C4C5C3C1C5C42)CCCn1ccnc1] |
| C36 | 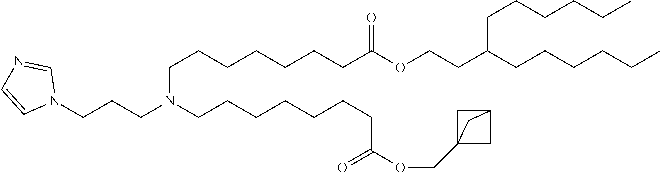 [SMILES: CCCCCCC(CCCCCC)CCOC(=O)CCCCCCCN(CCCCCCCC(=O)OCC12CC(C1)C2)CCCn1ccnc1] |
| C37 | 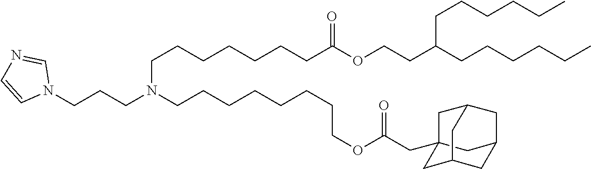 [SMILES: CCCCCCC(CCCCCC)CCOC(=O)CCCCCCCN(CCCCCCCCOC(=O)CC12CC3CC(CC(C3)C1)C2)CCCn1ccnc1] |
| C38 | 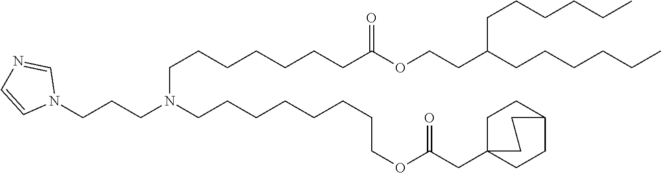 [SMILES: CCCCCCC(CCCCCC)CCOC(=O)CCCCCCCN(CCCCCCCCOC(=O)CC12CCC(CC1)CC2)CCCn1ccnc1] |
| C39 | 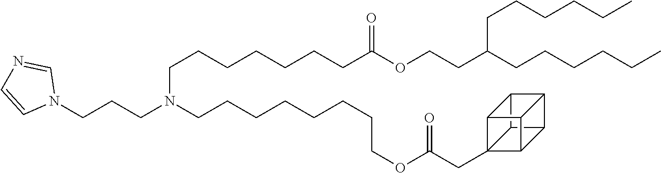 [SMILES: CCCCCCC(CCCCCC)CCOC(=O)CCCCCCCN(CCCCCCCCOC(=O)CC12C3C4C5C3C1C5C42)CCCn1ccnc1] |
| C40 | 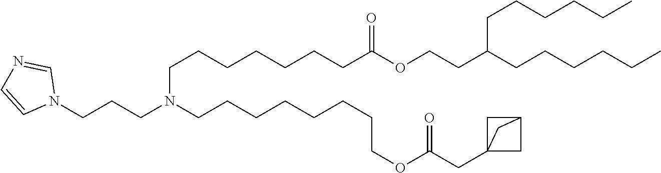 [SMILES: CCCCCCC(CCCCCC)CCOC(=O)CCCCCCCN(CCCCCCCCOC(=O)CC12CC(C1)C2)CCCn1ccnc1] |
| C41 | 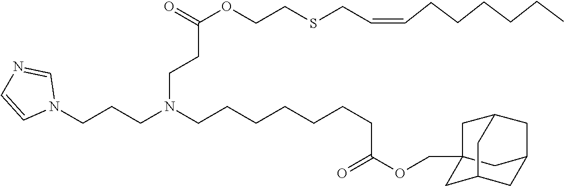 [SMILES: CCCCCC/C=C\CSCCOC(=O)CCN(CCCCCCCC(=O)OCC12CC3CC(CC(C3)C1)C2)CCCn1ccnc1] |
| C42 | 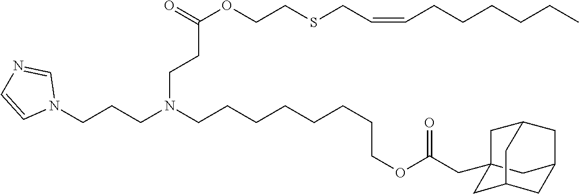 [SMILES: CCCCCC/C=C\CSCCOC(=O)CCN(CCCCCCCCOC(=O)CC12CC3CC(CC(C3)C1)C2)CCCn1ccnc1] |
| C43 | 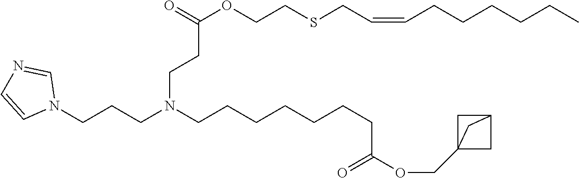 [SMILES: CCCCCC/C=C\CSCCOC(=O)CCN(CCCCCCCC(=O)OCC12CC(C1)C2)CCCn1ccnc1] |
| C44 | 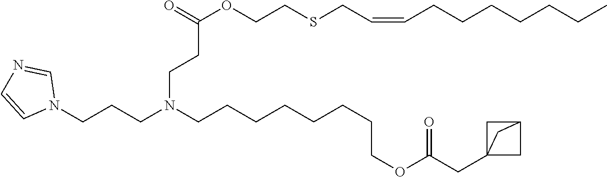 [SMILES: CCCCCCC/C=C\CSCCOC(=O)CCN(CCCCCCCCOC(=O)CC12CC(C1)C2)CCCn1ccnc1] |
| C45 | 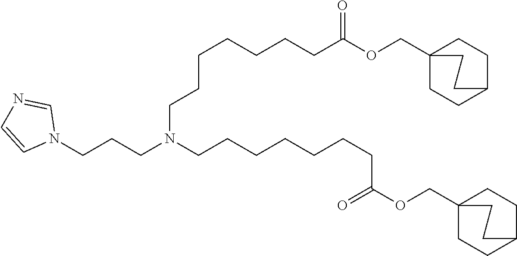 [SMILES: O=C(CCCCCCCN(CCCCCCCC(=O)OCC12CCC(CC1)CC2)CCCn1ccnc1)OCC12CCC(CC1)CC2] |
| C46 | 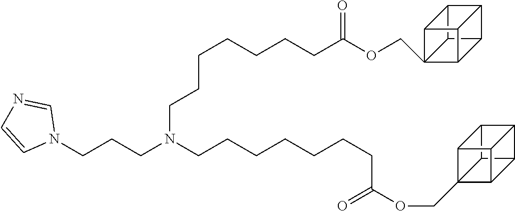 [SMILES: O=C(CCCCCCCN(CCCCCCCC(=O)OCC12C3C4C5C3C1C5C42)CCCn1ccnc1)OCC12C3C4C5C3C1C5C42] |
| C47 | 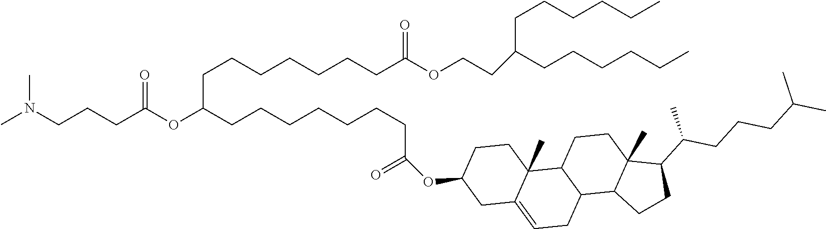 |
| C48 | 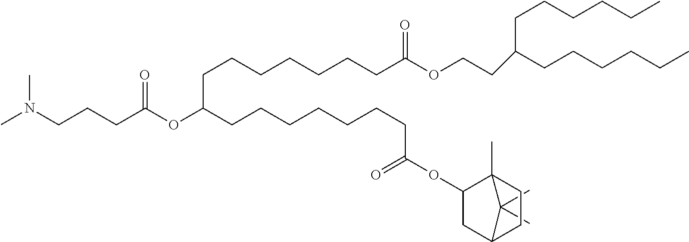 [SMILES: CCCCCCC(CCCCCC)CCOC(=O)CCCCCCCC(CCCCCCCC(=O)OC1CC2CCC1(C)C2(C)C)OC(=O)CCCN(C)C] |
| C49 | 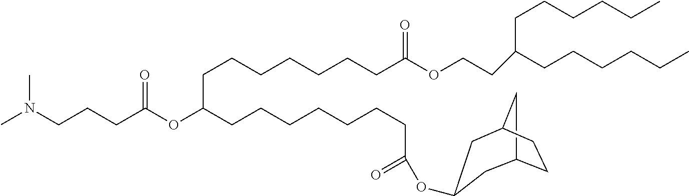 [SMILES: CCCCCCC(CCCCCC)CCOC(=O)CCCCCCCC(CCCCCCCC(=O)OC1CC2CCC(C2)C1)OC(=O)CCCN(C)C] |
| C50 | 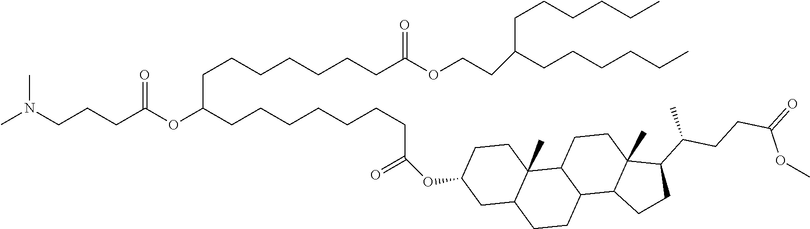 |
| C51 | 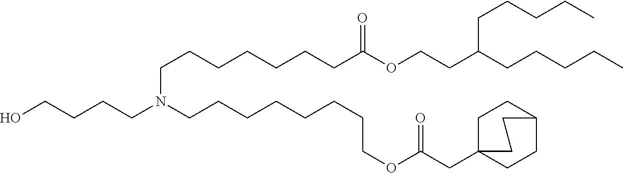 [SMILES: CCCCCC(CCCCC)CCOC(=O)CCCCCCCN(CCCCO)CCCCCCCCOC(=O)CC12CCC(CC1)CC2] |
| C52 | 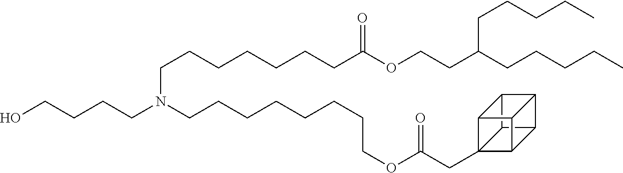 [SMILES: CCCCCC(CCCCC)CCOC(=O)CCCCCCCN(CCCCO)CCCCCCCCOC(=O)CC12C3C4C5C3C1C5C42] |
| C53 | 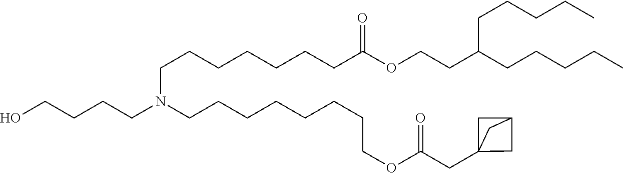 [SMILES: CCCCCC(CCCCC)CCOC(=O)CCCCCCCN(CCCCO)CCCCCCCCOC(=O)CC12CC(C1)C2] |
| C54 | 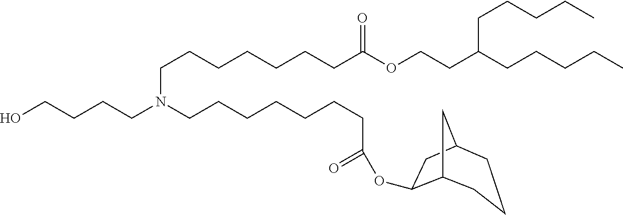 [SMILES: CCCCCC(CCCCC)CCOC(=O)CCCCCCCN(CCCCO)CCCCCCCC(=O)OC1CC2CCCC1C2] |
| C55 | 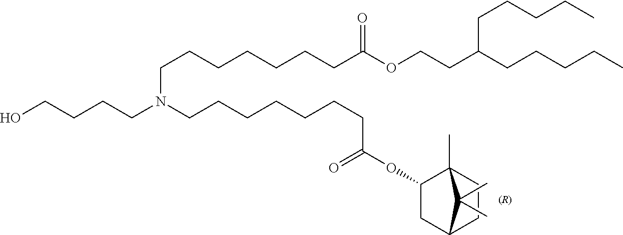 |
| C56 | 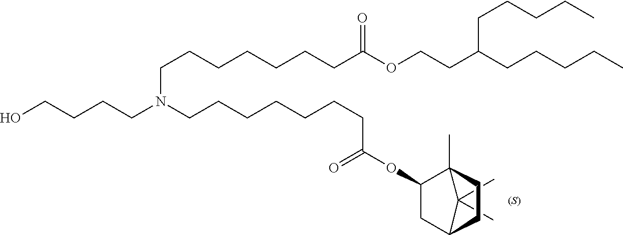 |
| C57 | 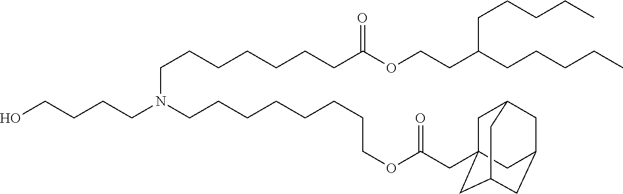 [SMILES: CCCCCC(CCCCC)CCOC(=O)CCCCCCCN(CCCCO)CCCCCCCCOC(=O)CC12CC3CC(CC(C3)C1)C2] |
| C58 | 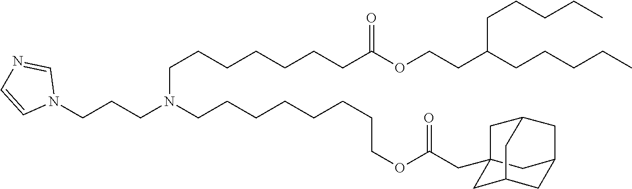 [SMILES: CCCCCC(CCCCC)CCOC(=O)CCCCCCCN(CCCCCCCCOC(=O)CC12CC3CC(CC(C3)C1)C2)CCCn1ccnc1] |
| C59 | 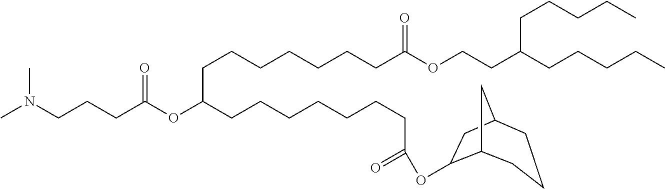 [SMILES: CCCCCC(CCCCC)CCOC(=O)CCCCCCCC(CCCCCCCC(=O)OC1CC2CCCC1C2)OC(=O)CCCN(C)C] |
| C60 | 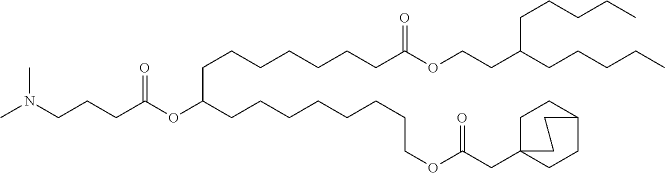 [SMILES: CCCCCC(CCCCC)CCOC(=O)CCCCCCCC(CCCCCCCCOC(=O)CC12CCC(CC1)CC2)OC(=O)CCCN(C)C] |
| C61 | 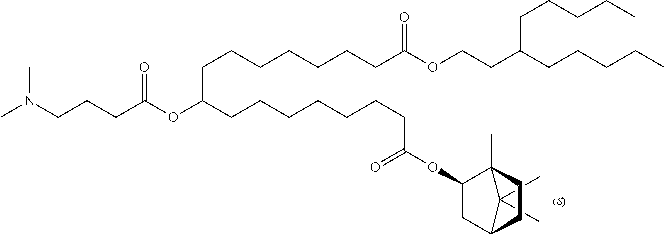 |
| C62 | 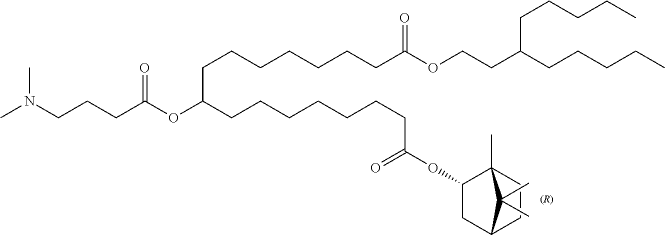 |
| C63 | 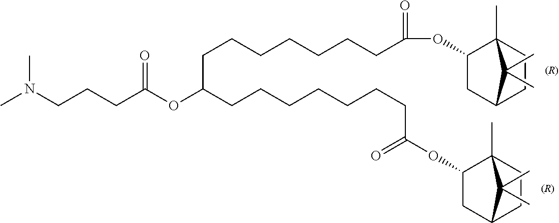 |
| C64 | 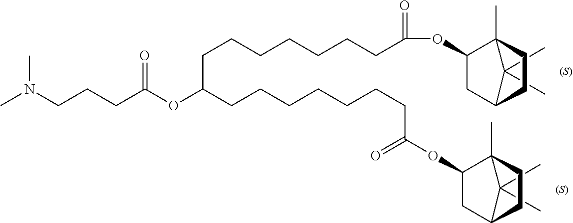 |
| C65 | 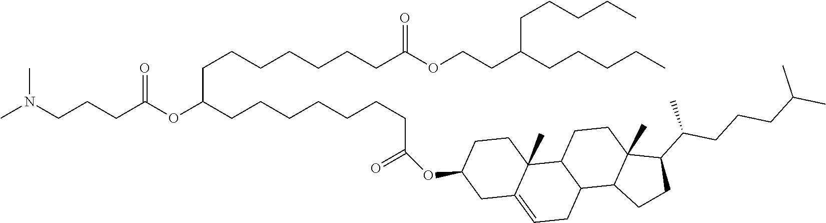 |
| C66 | 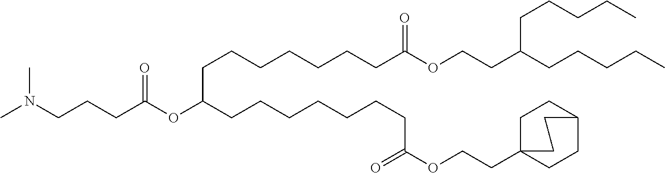 [SMILES: CCCCCC(CCCCC)CCOC(=O)CCCCCCCC(CCCCCCCC(=O)OCCC12CCC(CC1)CC2)OC(=O)CCCN(C)C] |
| C67 | 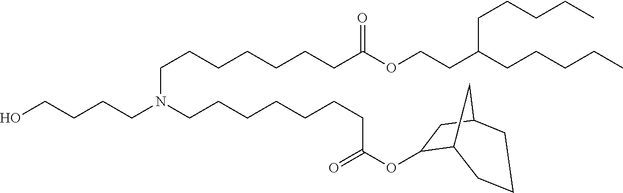 [SMILES: CCCCCC(CCCCC)CCOC(=O)CCCCCCCN(CCCCO)CCCCCCCC(=O)OC1CC2CCCC1C2] |
| C68 | 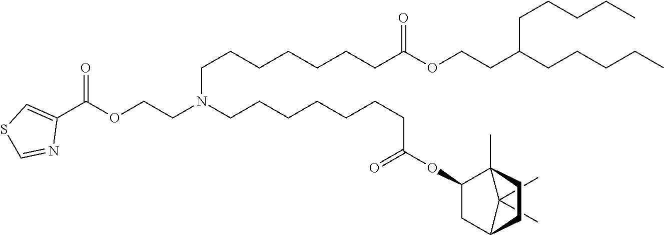 |
| C69 | 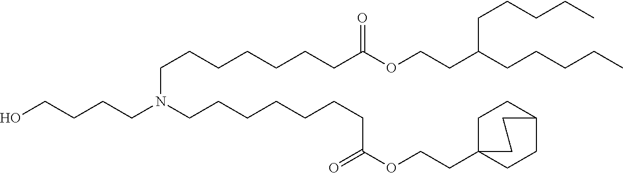 [SMILES: CCCCCC(CCCCC)CCOC(=O)CCCCCCCN(CCCCO)CCCCCCCC(=O)OCCC12CCC(CC1)CC2] |
| C70 | 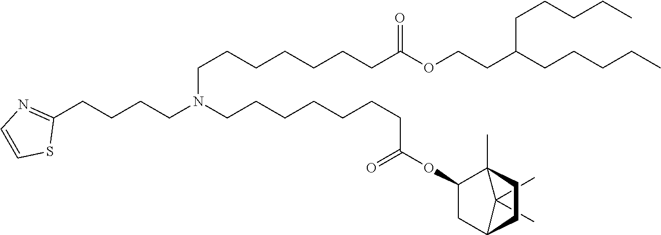 |
| C71 | 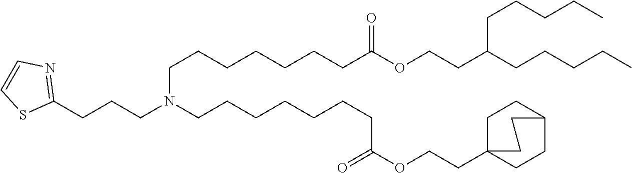 [SMILES: CCCCCC(CCCCC)CCOC(=O)CCCCCCCN(CCCCCCCC(=O)OCCC12CCC(CC1)CC2)CCCc1nccs1] |
| C72 | 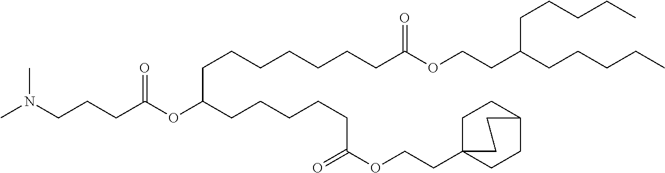 [SMILES: CCCCCC(CCCCC)CCOC(=O)CCCCCCCC(CCCCCC(=O)OCCC12CCC(CC1)CC2)OC(=O)CCCN(C)C] |
| C73 | 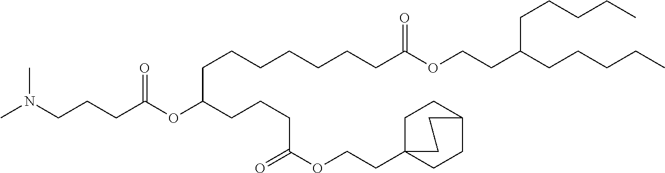 [SMILES: CCCCCC(CCCCC)CCOC(=O)CCCCCCCC(CCCC(=O)OCCC12CCC(CC1)CC2)OC(=O)CCCN(C)C] |
| C74 | 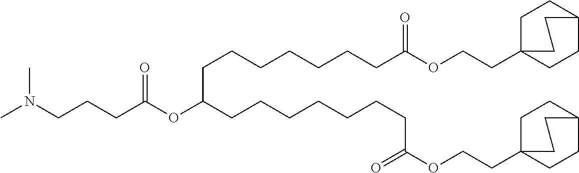 [SMILES: CN(C)CCCC(=O)OC(CCCCCCCC(=O)OCCC12CCC(CC1)CC2)CCCCCCCC(=O)OCCC12CCC(CC1)CC2] |
| C75 | 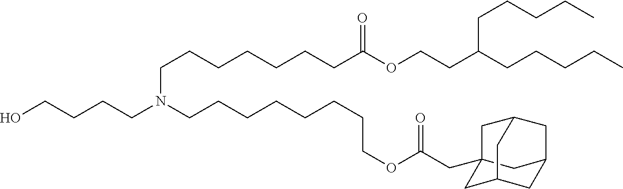 [SMILES: CCCCCC(CCCCC)CCOC(=O)CCCCCCCN(CCCCO)CCCCCCCCOC(=O)CC12CC3CC(CC(C3)C1)C2] |
| C76 | 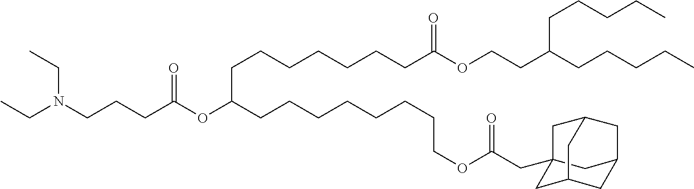 [SMILES: CCCCCC(CCCCC)CCOC(=O)CCCCCCCC(CCCCCCCCOC(=O)CC12CC3CC(CC(C3)C1)C2)OC(=O)CCCN(CC)CC] |
| C77 | 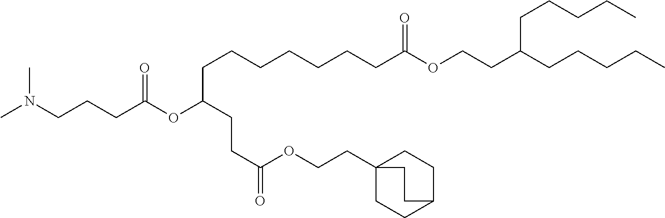 [SMILES: CCCCCC(CCCCC)CCOC(=O)CCCCCCCC(CCC(=O)OCCC12CCC(CC1)CC2)OC(=O)CCCN(C)C] |
| C78 | 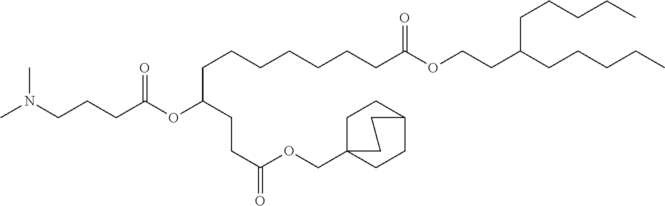 [SMILES: CCCCCC(CCCCC)CCOC(=O)CCCCCCCC(CCC(=O)OCC12CCC(CC1)CC2)OC(=O)CCCN(C)C] |
| C79 | 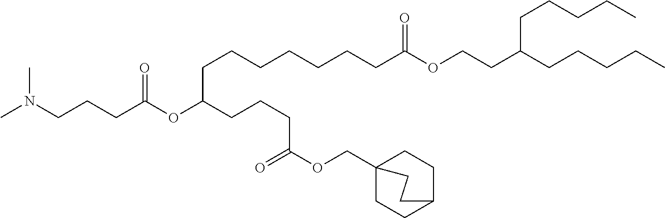 [SMILES: CCCCCC(CCCCC)CCOC(=O)CCCCCCCC(CCCC(=O)OCC12CCC(CC1)CC2)OC(=O)CCCN(C)C] |
| C80 | 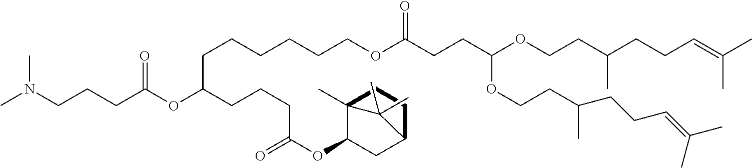 |
| C81 | 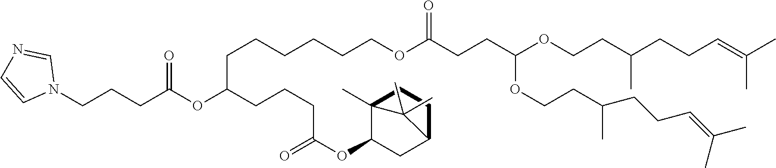 |

In some embodiments, an LNP of the present disclosure comprises an ionizable lipid disclosed in PCT Publication WO2023196931A1, which is incorporated by reference herein, in its entirety.

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CX-I):

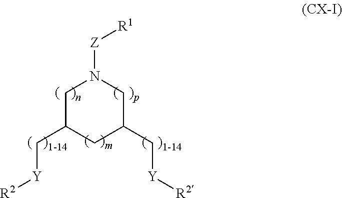

or a pharmaceutically acceptable salt thereof,
wherein
Z is selected from the group consisting of a bond,

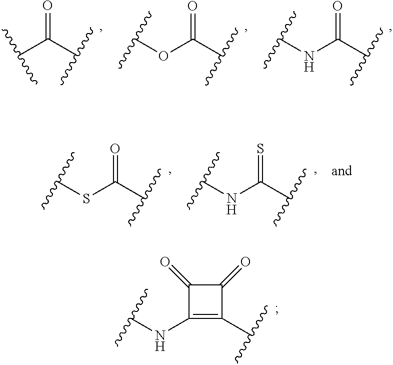

each Y is independently selected from the group consisting of

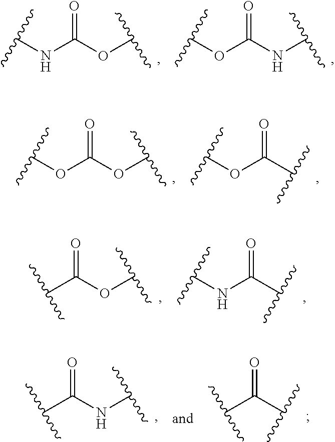

R¹ is —(CH₂)₁₋₆N(R″)₂ or —(CH₂)₁₋₆OH;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—; each R^{a} is independently optionally substituted C₁-C₆ alkyl; or
   two Ra are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-i):

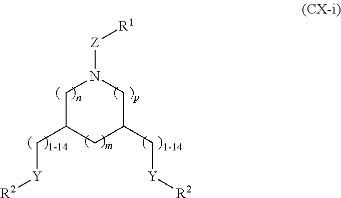

or a pharmaceutically acceptable salt thereof,
wherein
Z is selected from the group consisting of a bond,

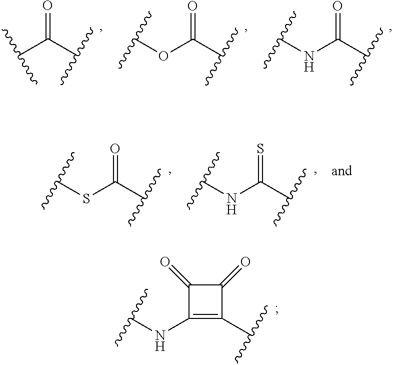

each Y is independently selected from the group consisting of

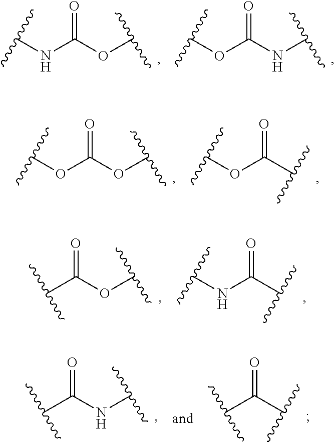

R¹ is —(CH₂)₁₋₆N(R^{a})₂;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R″ is independently optionally substituted C₁-C₆ alkyl; or
   two Ra are taken together, with the nitrogen on which they are attached, to form an optionally substituted 4-7 membered heterocyclyl ring;
m is 0, 1, or 2;
n is 1 or 2; and
p is 1 or 2.

In some embodiments, the present disclosure includes a compound selected from any lipid in Table (IV) below or a pharmaceutically acceptable salt thereof:

TABLE (IV)
Non-Limiting Examples of Ionizable Lipids

| | Compound |
|---|---|
| Structure | No. |
| 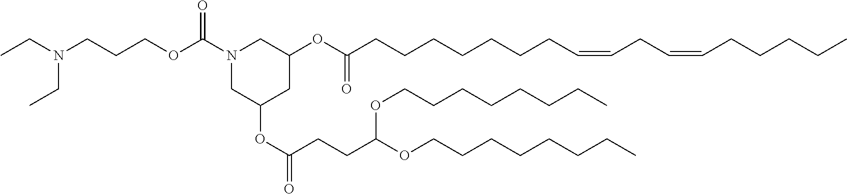 [SMILES: CCCCC/C=C\C/C=C\CCCCCCCC(=O)OC1CC(OC(=O)CCC(OCCCCCCCC)OCCCCCCCC)CN(C(=O)OCCCN(CC)CC)C1] | CX-1 |
| 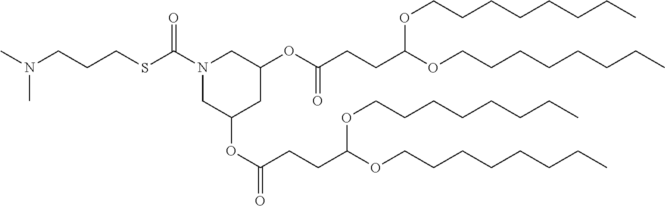 [SMILES: CCCCCCCCOC(CCC(=O)OC1CC(OC(=O)CCC(OCCCCCCCC)OCCCCCCCC)CN(C(=O)SCCCN(C)C)C1)OCCCCCCCC] | CX-2 |
| 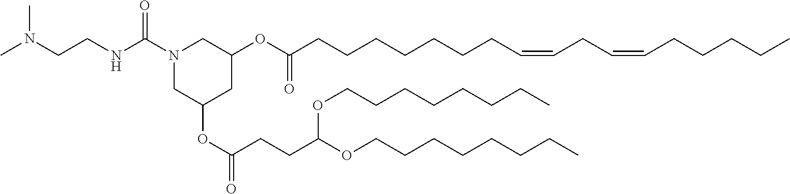 [SMILES: CCCCC/C=C\C/C=C\CCCCCCCC(=O)OC1CC(OC(=O)CCC(OCCCCCCCC)OCCCCCCCC)CN(C(=O)NCCN(C)C)C1] | CX-3 |
| 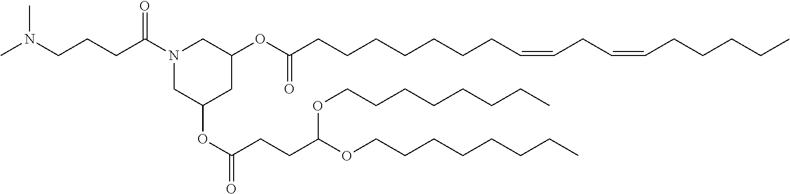 [SMILES: CCCCC/C=C\C/C=C\CCCCCCCC(=O)OC1CC(OC(=O)CCC(OCCCCCCCC)OCCCCCCCC)CN(C(=O)CCCN(C)C)C1] | CX-4 |
| 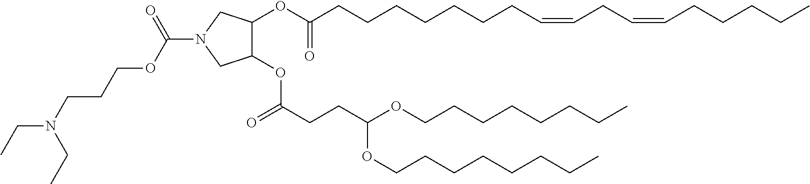 [SMILES: CCCCC/C=C\C/C=C\CCCCCCCC(=O)OC1CN(C(=O)OCCCN(CC)CC)CC1OC(=O)CCC(OCCCCCCCC)OCCCCCCCC] | CX-5 |
| 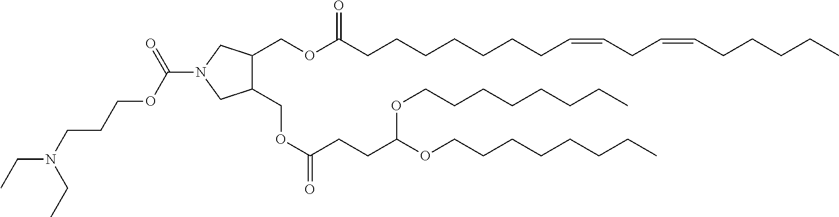 [SMILES: CCCCC/C=C\C/C=C\CCCCCCCC(=O)OCC1CN(C(=O)OCCCN(CC)CC)CC1COC(=O)CCC(OCCCCCCCC)OCCCCCCCC] | CX-6 |
| 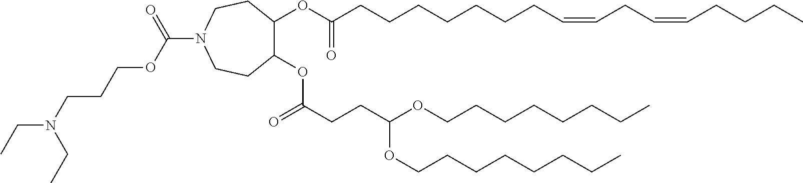 [SMILES: CCCC/C=C\C/C=C\CCCCCCCC(=O)OC1CCN(C(=O)OCCCN(CC)CC)CCC1OC(=O)CCC(OCCCCCCCC)OCCCCCCCC] | CX-7 |
| 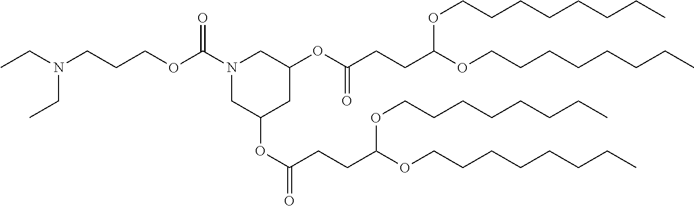 [SMILES: CCCCCCCCOC(CCC(=O)OC1CC(OC(=O)CCC(OCCCCCCCC)OCCCCCCCC)CN(C(=O)OCCCN(CC)CC)C1)OCCCCCCCC] | CX-8 |
| 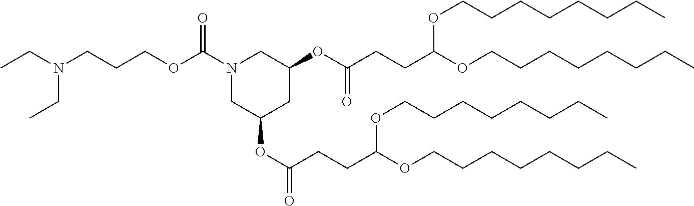 [SMILES: CCCCCCCCOC(CCC(=O)O[C@@H]1C[C@H](OC(=O)CCC(OCCCCCCCC)OCCCCCCCC)CN(C(=O)OCCCN(CC)CC)C1)OCCCCCCCC] | CX-8a |
| 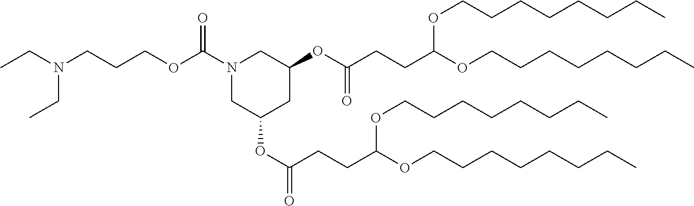 [SMILES: CCCCCCCCOC(CCC(=O)O[C@H]1C[C@H](OC(=O)CCC(OCCCCCCCC)OCCCCCCCC)CN(C(=O)OCCCN(CC)CC)C1)OCCCCCCCC] | CX-8b |
| 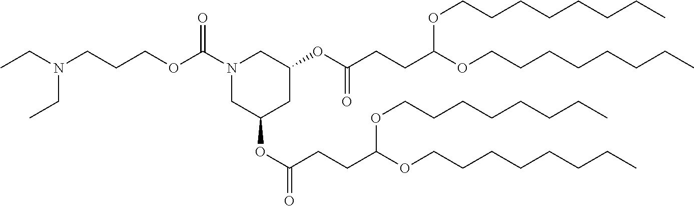 [SMILES: CCCCCCCCOC(CCC(=O)O[C@@H]1C[C@@H](OC(=O)CCC(OCCCCCCCC)OCCCCCCCC)CN(C(=O)OCCCN(CC)CC)C1)OCCCCCCCC] | CX-8c |
| 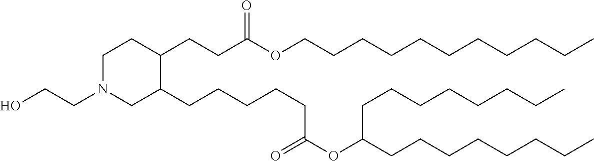 [SMILES: CCCCCCCCCCCOC(=O)CCC1CCN(CCO)CC1CCCCCC(=O)OC(CCCCCCCC)CCCCCCCC] | CX-9 |
| 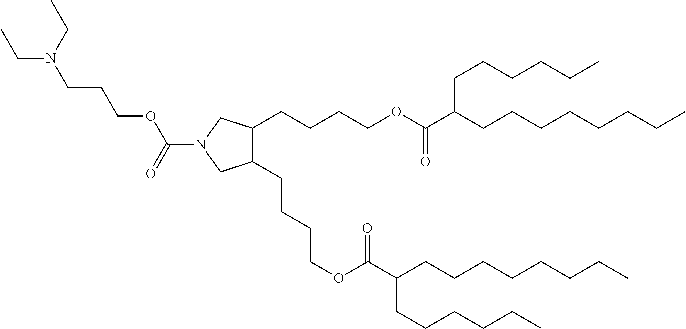 [SMILES: CCCCCCCCC(CCCCCC)C(=O)OCCCCC1CN(C(=O)OCCCN(CC)CC)CC1CCCCOC(=O)C(CCCCCC)CCCCCCCC] | CX-10 |
| 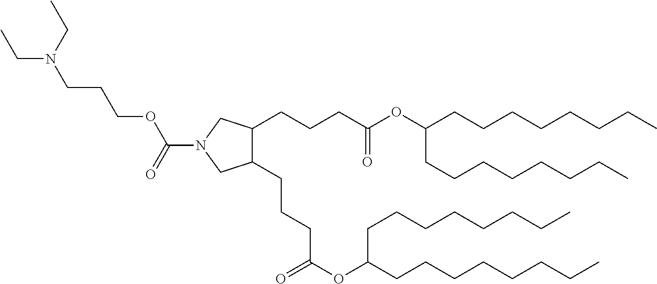 [SMILES: CCCCCCCCC(CCCCCCCC)OC(=O)CCCC1CN(C(=O)OCCCN(CC)CC)CC1CCCC(=O)OC(CCCCCCCC)CCCCCCCC] | CX-11 |
| 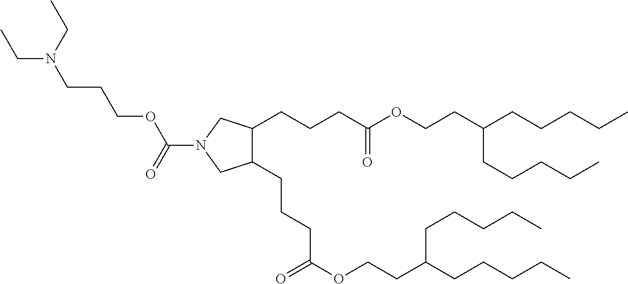 [SMILES: CCCCCC(CCCCC)CCOC(=O)CCCC1CN(C(=O)OCCCN(CC)CC)CC1CCCC(=O)OCCC(CCCCC)CCCCC] | CX-12 |
| 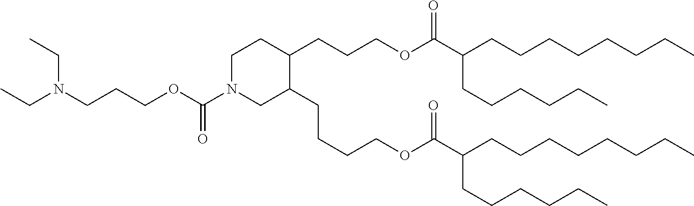 [SMILES: CCCCCCCCC(CCCCCC)C(=O)OCCCCC1CN(C(=O)OCCCN(CC)CC)CCC1CCCOC(=O)C(CCCCCC)CCCCCCCC] | CX-13 |
| 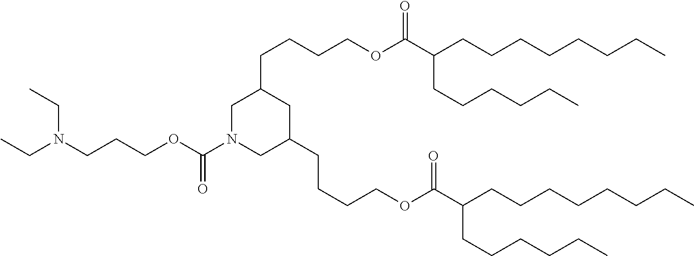 [SMILES: CCCCCCCCC(CCCCCC)C(=O)OCCCCC1CC(CCCCOC(=O)C(CCCCCC)CCCCCCCC)CN(C(=O)OCCCN(CC)CC)C1] | CX-14 |
| 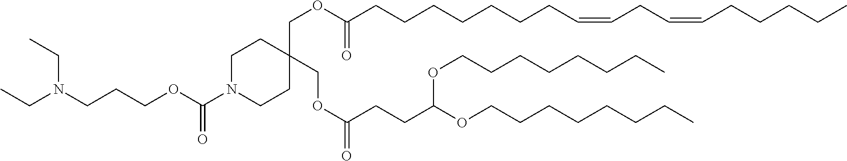 [SMILES: CCCCC/C=C\C/C=C\CCCCCCCC(=O)OCC1(COC(=O)CCC(OCCCCCCCC)OCCCCCCCC)CCN(C(=O)OCCCN(CC)CC)CC1] | CX-15 |
| 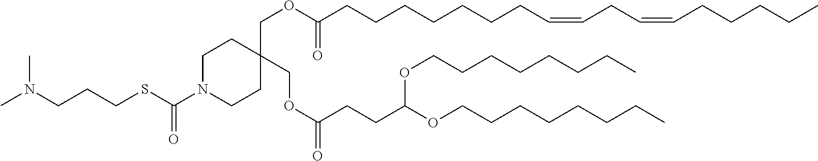 [SMILES: CCCCC/C=C\C/C=C\CCCCCCCC(=O)OCC1(COC(=O)CCC(OCCCCCCCC)OCCCCCCCC)CCN(C(=O)SCCCN(C)C)CC1] | CX-16 |
| 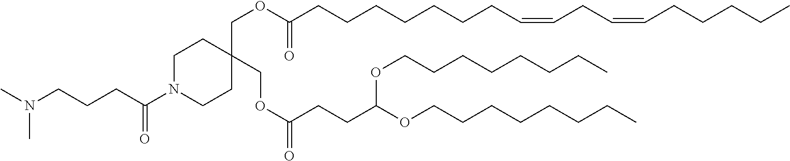 [SMILES: CCCCC/C=C\C/C=C\CCCCCCCC(=O)OCC1(COC(=O)CCC(OCCCCCCCC)OCCCCCCCC)CCN(C(=O)CCCN(C)C)CC1] | CX-17 |
| 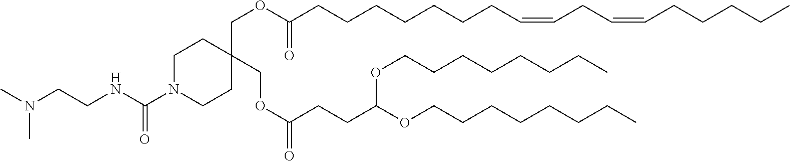 [SMILES: CCCCC/C=C\C/C=C\CCCCCCCC(=O)OCC1(COC(=O)CCC(OCCCCCCCC)OCCCCCCCC)CCN(C(=O)NCCN(C)C)CC1] | CX-18 |
| 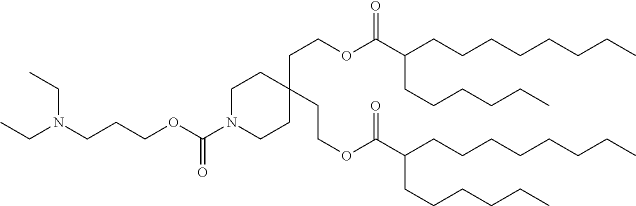 [SMILES: CCCCCCCCC(CCCCCC)C(=O)OCCC1(CCOC(=O)C(CCCCCC)CCCCCCCC)CCN(C(=O)OCCCN(CC)CC)CC1] | CX-19 |
| 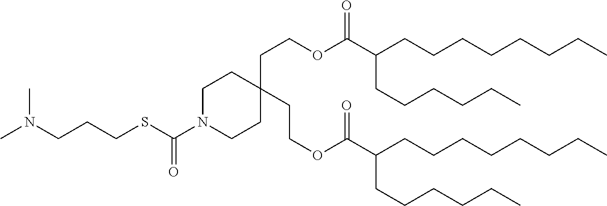 [SMILES: CCCCCCCCC(CCCCCC)C(=O)OCCC1(CCOC(=O)C(CCCCCC)CCCCCCCC)CCN(C(=O)SCCCN(C)C)CC1] | CX-20 |
| 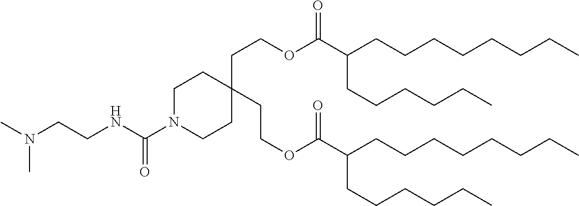 [SMILES: CCCCCCCCC(CCCCCC)C(=O)OCCC1(CCOC(=O)C(CCCCCC)CCCCCCCC)CCN(C(=O)NCCN(C)C)CC1] | CX-21 |
| 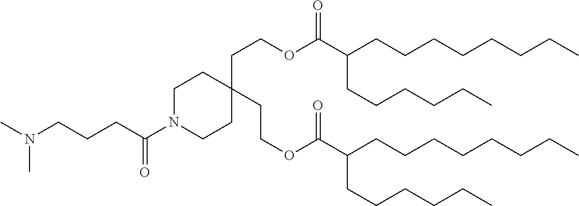 [SMILES: CCCCCCCCC(CCCCCC)C(=O)OCCC1(CCOC(=O)C(CCCCCC)CCCCCCCC)CCN(C(=O)CCCN(C)C)CC1] | CX-22 |
| 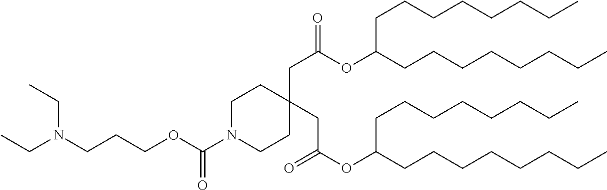 [SMILES: CCCCCCCCC(CCCCCCCC)OC(=O)CC1(CC(=O)OC(CCCCCCCC)CCCCCCCC)CCN(C(=O)OCCCN(CC)CC)CC1] | CX-23 |
| 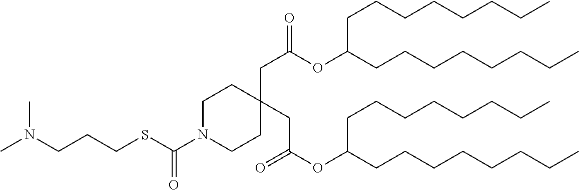 [SMILES: CCCCCCCCC(CCCCCCCC)OC(=O)CC1(CC(=O)OC(CCCCCCCC)CCCCCCCC)CCN(C(=O)SCCCN(C)C)CC1] | CX-24 |
| 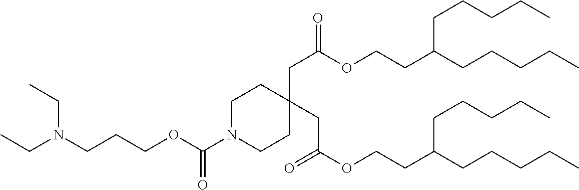 [SMILES: CCCCCC(CCCCC)CCOC(=O)CC1(CC(=O)OCCC(CCCCC)CCCCC)CCN(C(=O)OCCCN(CC)CC)CC1] | CX-25 |
| 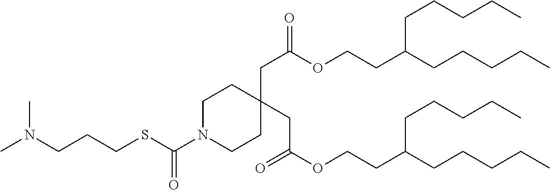 [SMILES: CCCCCC(CCCCC)CCOC(=O)CC1(CC(=O)OCCC(CCCCC)CCCCC)CCN(C(=O)SCCCN(C)C)CC1] | CX-26 |
| 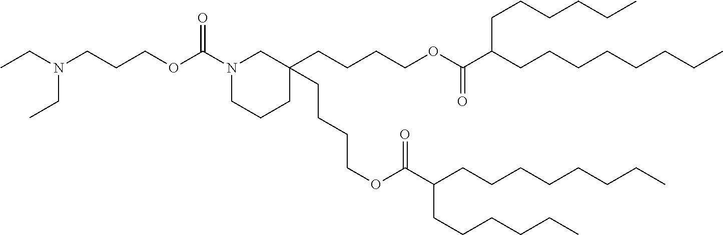 [SMILES: CCCCCCCCC(CCCCCC)C(=O)OCCCCC1(CCCCOC(=O)C(CCCCCC)CCCCCCCC)CCCN(C(=O)OCCCN(CC)CC)C1] | CX-27 |
| 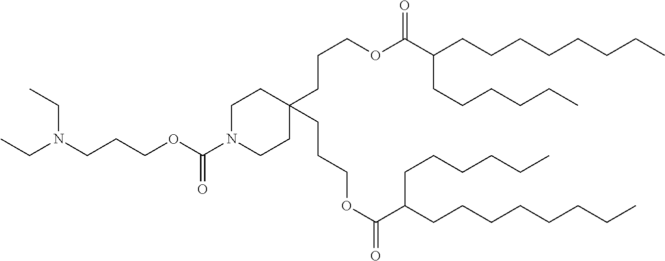 [SMILES: CCCCCCCCC(CCCCCC)C(=O)OCCCC1(CCCOC(=O)C(CCCCCC)CCCCCCCC)CCN(C(=O)OCCCN(CC)CC)CC1] | CX-28 |
| 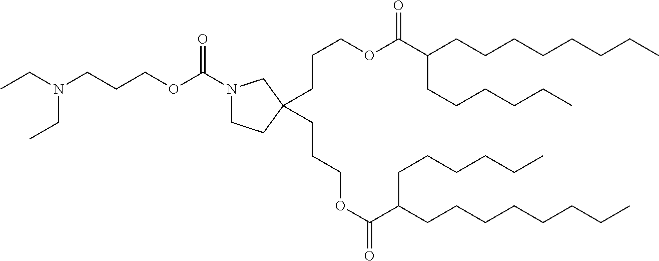 [SMILES: CCCCCCCCC(CCCCCC)C(=O)OCCCC1(CCCOC(=O)C(CCCCCC)CCCCCCCC)CCN(C(=O)OCCCN(CC)CC)C1] | CX-29 |
| 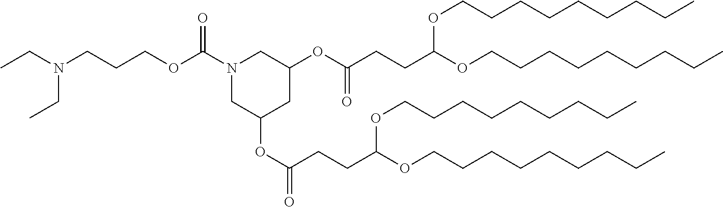 [SMILES: CCCCCCCCCOC(CCC(=O)OC1CC(OC(=O)CCC(OCCCCCCCCC)OCCCCCCCCC)CN(C(=O)OCCCN(CC)CC)C1)OCCCCCCCCC] | CX-30 |
| 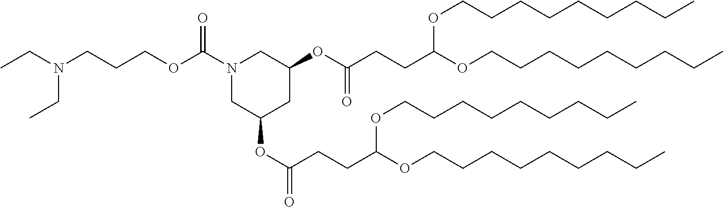 [SMILES: CCCCCCCCCOC(CCC(=O)O[C@@H]1C[C@H](OC(=O)CCC(OCCCCCCCCC)OCCCCCCCCC)CN(C(=O)OCCCN(CC)CC)C1)OCCCCCCCCC] | CX-30a |
| 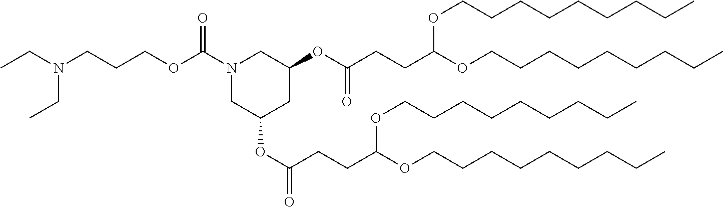 [SMILES: CCCCCCCCCOC(CCC(=O)O[C@H]1C[C@H](OC(=O)CCC(OCCCCCCCCC)OCCCCCCCCC)CN(C(=O)OCCCN(CC)CC)C1)OCCCCCCCCC] | CX-30b |
| 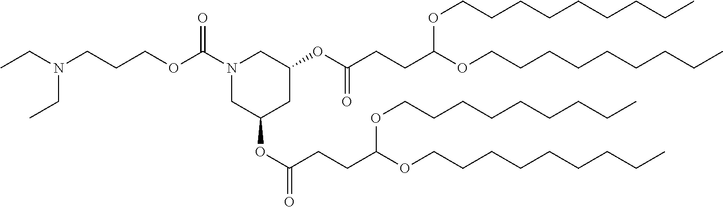 [SMILES: CCCCCCCCCOC(CCC(=O)O[C@@H]1C[C@@H](OC(=O)CCC(OCCCCCCCCC)OCCCCCCCCC)CN(C(=O)OCCCN(CC)CC)C1)OCCCCCCCCC] | CX-30c |

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CZ-J)

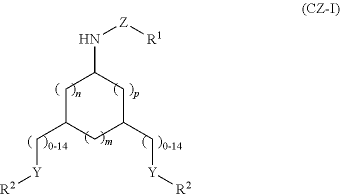

or a pharmaceutically acceptable salt thereof,
wherein
Z is selected from the group consisting of a bond,

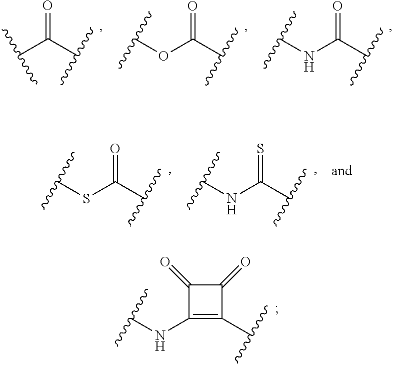

each Y is independently selected from the group consisting of,

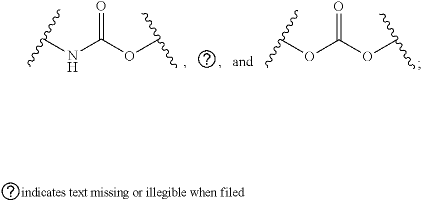 [SMILES: *NC(=O)O* ; *OC(=O)O*]

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, the present disclosure includes a compound selected from any lipid in Table (V) below or a pharmaceutically acceptable salt thereof:

TABLE (V)
Non-Limiting Examples of Ionizable Lipids

| | Compound |
|---|---|
| Structure | No. |
| 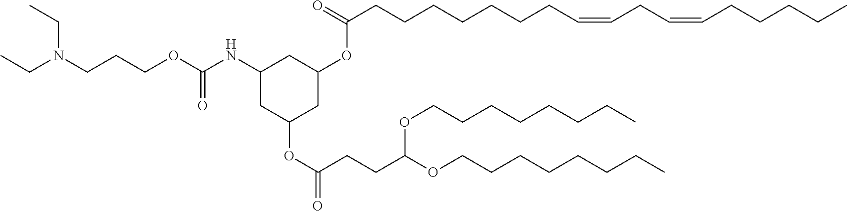 [SMILES: CCCCC/C=C\C/C=C\CCCCCCCC(=O)OC1CC(NC(=O)OCCCN(CC)CC)CC(OC(=O)CCC(OCCCCCCCC)OCCCCCCCC)C1] | CZ-1 |
| 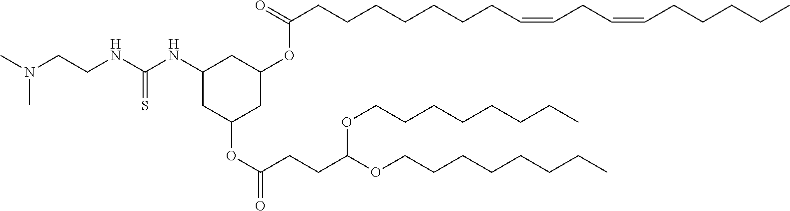 [SMILES: CCCCC/C=C\C/C=C\CCCCCCCC(=O)OC1CC(NC(=S)NCCN(C)C)CC(OC(=O)CCC(OCCCCCCCC)OCCCCCCCC)C1] | CZ-2 |
| 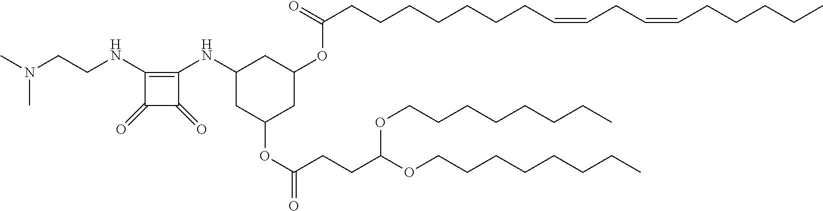 [SMILES: CCCCC/C=C\C/C=C\CCCCCCCC(=O)OC1CC(Nc2c(NCCN(C)C)c(=O)c2=O)CC(OC(=O)CCC(OCCCCCCCC)OCCCCCCCC)C1] | CZ-3 |
| 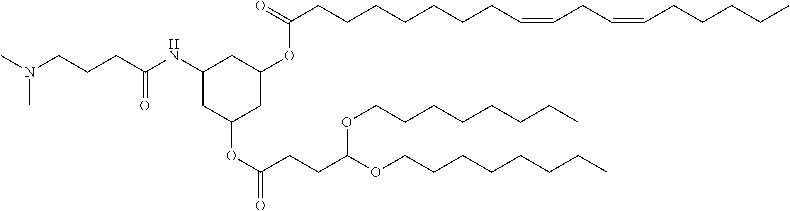 [SMILES: CCCCC/C=C\C/C=C\CCCCCCCC(=O)OC1CC(NC(=O)CCCN(C)C)CC(OC(=O)CCC(OCCCCCCCC)OCCCCCCCC)C1] | CZ-4 |
| 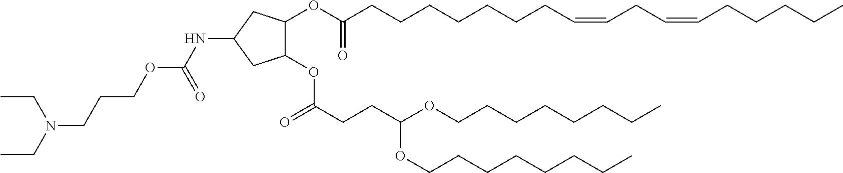 [SMILES: CCCCC/C=C\C/C=C\CCCCCCCC(=O)OC1CC(NC(=O)OCCCN(CC)CC)CC1OC(=O)CCC(OCCCCCCCC)OCCCCCCCC] | CZ-5 |
| 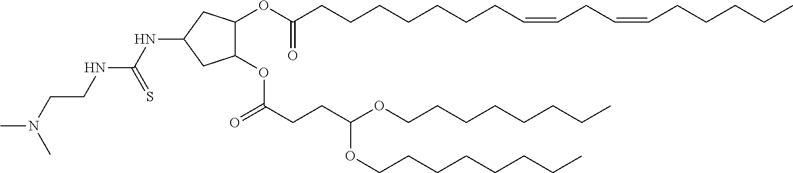 [SMILES: CCCCC/C=C\C/C=C\CCCCCCCC(=O)OC1CC(NC(=S)NCCN(C)C)CC1OC(=O)CCC(OCCCCCCCC)OCCCCCCCC] | CZ-6 |
| 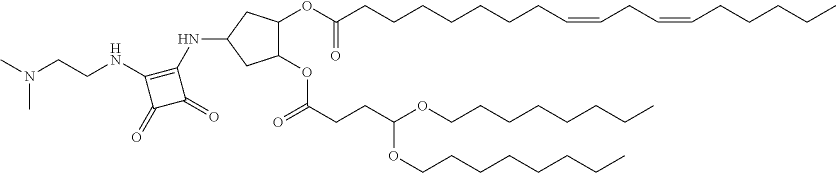 [SMILES: CCCCC/C=C\C/C=C\CCCCCCCC(=O)OC1CC(Nc2c(NCCN(C)C)c(=O)c2=O)CC1OC(=O)CCC(OCCCCCCCC)OCCCCCCCC] | CZ-7 |
| 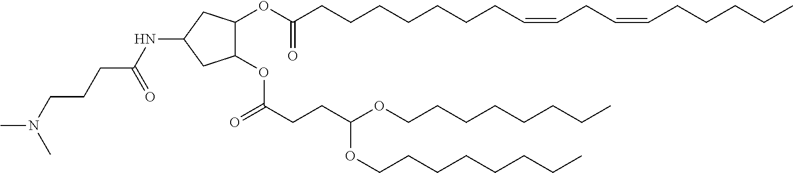 [SMILES: CCCCC/C=C\C/C=C\CCCCCCCC(=O)OC1CC(NC(=O)CCCN(C)C)CC1OC(=O)CCC(OCCCCCCCC)OCCCCCCCC] | CZ-8 |
| 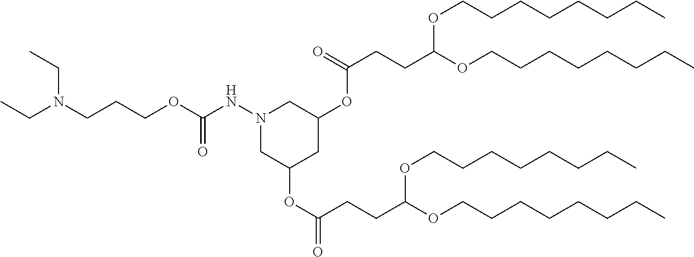 [SMILES: CCCCCCCCOC(CCC(=O)OC1CC(OC(=O)CCC(OCCCCCCCC)OCCCCCCCC)CN(NC(=O)OCCCN(CC)CC)C1)OCCCCCCCC] | CZ-9 |
| 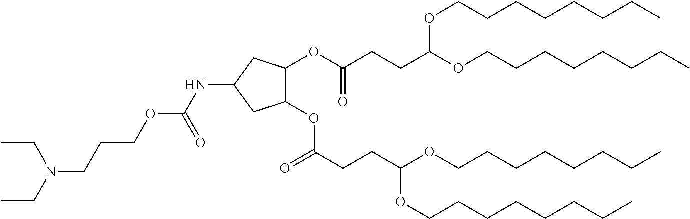 [SMILES: CCCCCCCCOC(CCC(=O)OC1CC(NC(=O)OCCCN(CC)CC)CC1OC(=O)CCC(OCCCCCCCC)OCCCCCCCC)OCCCCCCCC] | CZ-10 |
| 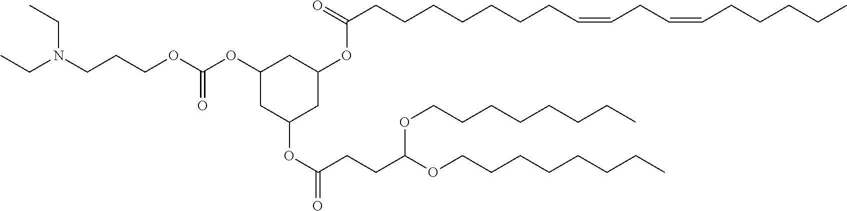 [SMILES: CCCCC/C=C\C/C=C\CCCCCCCC(=O)OC1CC(OC(=O)CCC(OCCCCCCCC)OCCCCCCCC)CC(OC(=O)OCCCN(CC)CC)C1] | CZ-11 |
| 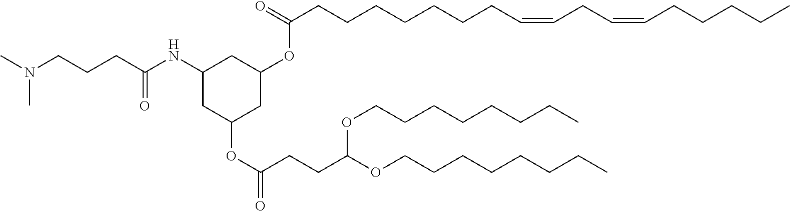 [SMILES: CCCCC/C=C\C/C=C\CCCCCCCC(=O)OC1CC(NC(=O)CCCN(C)C)CC(OC(=O)CCC(OCCCCCCCC)OCCCCCCCC)C1] | CZ-12 |
| 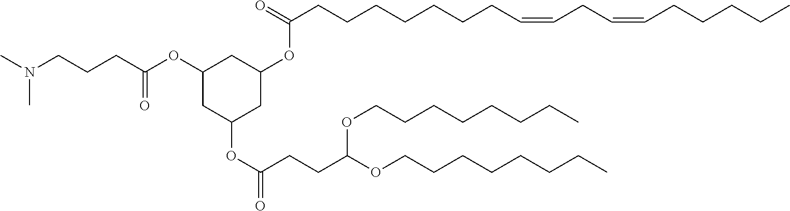 [SMILES: CCCCC/C=C\C/C=C\CCCCCCCC(=O)OC1CC(OC(=O)CCCN(C)C)CC(OC(=O)CCC(OCCCCCCCC)OCCCCCCCC)C1] | CZ-13 |
| 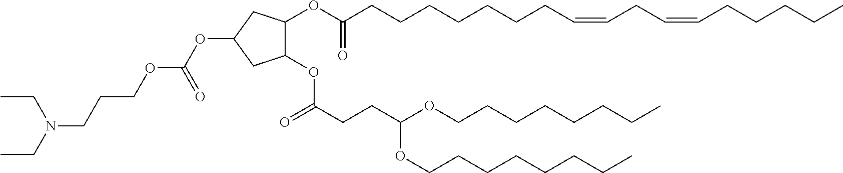 [SMILES: CCCCC/C=C\C/C=C\CCCCCCCC(=O)OC1CC(OC(=O)OCCCN(CC)CC)CC1OC(=O)CCC(OCCCCCCCC)OCCCCCCCC] | CZ-14 |
| 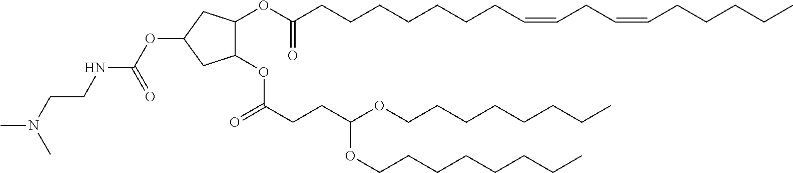 [SMILES: CCCCC/C=C\C/C=C\CCCCCCCC(=O)OC1CC(OC(=O)NCCN(C)C)CC1OC(=O)CCC(OCCCCCCCC)OCCCCCCCC] | CZ-15 |
| 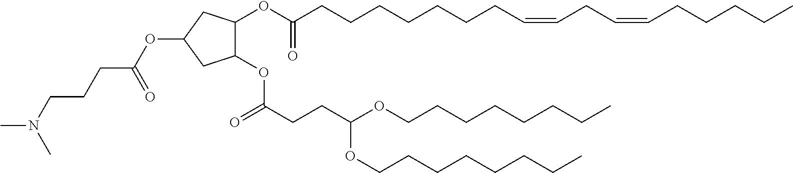 [SMILES: CCCCC/C=C\C/C=C\CCCCCCCC(=O)OC1CC(OC(=O)CCCN(C)C)CC1OC(=O)CCC(OCCCCCCCC)OCCCCCCCC] | CZ-16 |
| 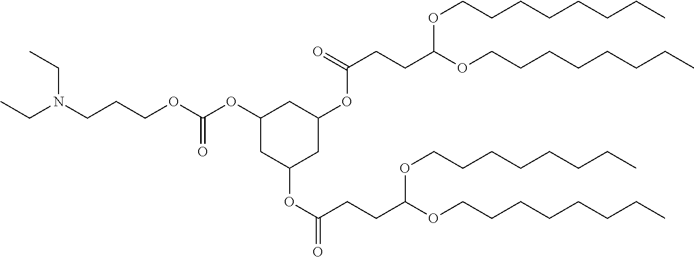 [SMILES: CCCCCCCCOC(CCC(=O)OC1CC(OC(=O)CCC(OCCCCCCCC)OCCCCCCCC)CC(OC(=O)OCCCN(CC)CC)C1)OCCCCCCCC] | CZ-17 |
| 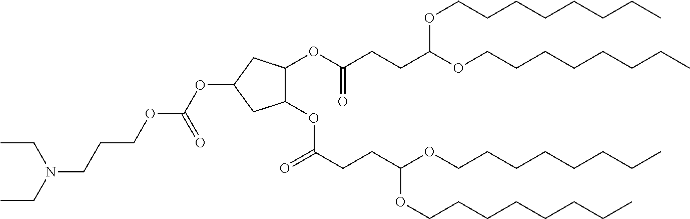 [SMILES: CCCCCCCCOC(CCC(=O)OC1CC(OC(=O)OCCCN(CC)CC)CC1OC(=O)CCC(OCCCCCCCC)OCCCCCCCC)OCCCCCCCC] | CZ-18 |

ii. Structural Lipids

In some embodiments, an LNP comprises a structural lipid. Structural lipids can be selected from the group consisting of, but are not limited to, cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and mixtures thereof. In some embodiments, the structural lipid is cholesteryl hemisuccinate (CHEMS). In some embodiments, the structural lipid is 3-(4-((2-(4-morpholinyl)ethyl)amino)-4-oxobutanoate) (Mochol). In some embodiments, the structural lipid is cholesterol. In some embodiments, the structural lipid is a cholesterol analogue disclosed by Patel, et al., Nat Commun., 11, 983 (2020), which is incorporated herein by reference in its entirety. In some embodiments, the structural lipid includes cholesterol and a corticosteroid (such as prednisolone, dexamethasone, prednisone, and hydrocortisone), or any combinations thereof. In some embodiments, a structural lipid is described in international patent application WO2019152557A1, which is incorporated herein by reference in its entirety.

In some embodiments, a structural lipid is a cholesterol analog. Using a cholesterol analog may enhance endosomal escape as described in Patel et al., Naturally-occurring cholesterol analogues in lipid nanoparticles induce polymorphic shape and enhance intracellular delivery of mRNA, Nature Communications (2020), which is incorporated herein by reference.

In some embodiments, a structural lipid is a phytosterol. Using a phytosterol may enhance endosomal escape as described in Herrera et al., Illuminating endosomal escape of polymorphic lipid nanoparticles that boost mRNA delivery, Biomaterials Science (2020), which is incorporated herein by reference.

In some embodiments, a structural lipid contains plant sterol mimetics for enhanced endosomal release.

iii. PEGylated Lipids

A PEGylated lipid is a lipid modified with polyethylene glycol.

In some embodiments, an LNP comprises one, two or more PEGylated lipid or PEG-modified lipid. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; or WO 2019/089828A1, each of which is incorporated by reference herein in their entirety.

In some embodiments, the LNP comprises a PEGylated lipid disclosed and described in PCT Publication WO2024044728A1, which is incorporated by reference herein, in its entirety. In certain embodiments, the PEGylated lipid is a lipid of any one of formulas PL-I′, PL-I″, PL-I, PL-Ia, PL-Ib, PL-Iaa, PL-Iab, PL-Iac, PL-Iad, PL-Iae, PL-Iaf, PL-Iag, PL-Iah, PL-Iba, PL-Ibb, PL-Ibc, PL-Ibd, PL-Ibe, PL-Ibf, PL-Ibg, PL-Ibh, PL-Ica, PL-Icb, PL-Ice, PL-Icd, PL-Id PL-Ie, PL-If, PL-Ig, PL-Ih, PL-Ii, PL-Iha, PL-Ihb, PL-Ihc, PL-Ihd, PL-Iia, PL-Iib, PL-Iic, PL-Iid, PL-Ij, PL-Ik, L-Il, PL-Im, PL-In, PL-Io, PL-Ip, PL-Iq, PL-Ioa, PL-Iob, PL-Ioc, PL-Iod, PL-Ioe, PL-Iof, PL-Iog, PL-Ioh, PL-Ipa, PL-Ipb, PL-Ipc, PL-Ipd, PL-Ipe, PL-Ipf, PL-Ipg, PL-Iph, PL-Iqa, PL-Iqb, PL-Iqc, PL-Iqd, PL-Ir, PL-Is, PL-It, PL-Iu, PL-Iv, PL-Iw, PL-Iva, PL-Ivb, PL-Ivc, PL-Ivd, PL-Iwa, PL-Iwb, PL-Iwc, PL-Iwd, PL-Ix, PL-Ixx, PL-Iy, PL-Iyy, PL-Iyyy, PL-Iz, PL-Izz, PL-Izzz, PL-II′, PL-II″, PL-II, PL-IIc, PL-IId, PL-IIe, PL-IIf, PL-IIg, PL-IIh, PL-IIa, PL-IIb, PL-IIk, PL-IIm or PL-IIn.

In some embodiments, the PEGylated lipid is a compound of formula PL-I′:

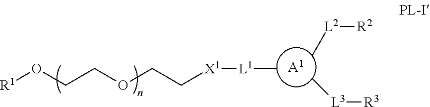

or a pharmaceutically acceptable salt thereof, wherein:
A¹ is a saturated 5-6 membered carbocyclic ring or a saturated 5-6 membered heterocyclic ring containing 1 or 2 heteroatoms independently selected from nitrogen, oxygen, and sulfur, wherein the carbocyclic ring and heterocyclic ring are substituted with t occurrences of R⁴;
X¹ is —N(H)—, —N(C₁₋₆ alkyl)-, —C₁₋₆ aliphatic-N(H)—, —C₁₋₆ aliphatic-N(C₁₋₆ alkyl)—, —O— or —C₁₋₆ aliphatic-O—;
L¹ is —C(O)(C₁₋₆ aliphatic)C(O)—N(R)—, —C(O)(C₁₋₆ aliphatic)—N(R)C(O)—, —C(O)(C₁₋₆ aliphatic)C(O)O—, —C(O)(C₁₋₆ aliphatic)C(O)—, —C(O)(C₁₋₆ aliphatic)C(O)OCH₂—, —C(O)(C₁₋₆ aliphatic)—, —C(O)(C₁₋₆ aliphatic)—N(R)—, or —C(O)—;
L² and L³ are independently a covalent bond or C₁₋₆ alkylene wherein one methylene unit of the C₁₋₆ alkylene is optionally replaced with —O—, —NR—, —S—, —S—S—, —S(O)—, —S(O)₂—, —C(O)—, —C(O)O—, —OC(O)—, —OC(O)O—, —OC(O)N(R)—, —N(R)C(O)O—, —C(O)N(R)—, —N(R)C(O)—, —N(R)C(O)N(R)—, —C(R⁵)═N—, or —C(R⁵)═N—O—;
R¹ is H, C₁₋₆ alkyl, —(C₁₋₆ alkyl)-N₃, —(C₁₋₆ alkyl)-SH, or C₃₋₈ alkynyl;
R² and R³ are independently a straight or branched C₆₋₃₀ alkyl, straight or branched C₆₋₃₀ alkenyl, or straight or branched C₆₋₃₀ alkynyl; wherein 1, 2, or 3 methylene units are independently and optionally replaced by a saturated or partially unsaturated C₃₋₆ carbocyclic ring or phenylene; wherein the alkyl, alkenyl, and alkynyl and any carbocyclic ring or phenylene is substituted with m instances of R^{x};
R⁴ is C₁₋₄ alkyl;
R⁵ is C₁₋₆ alkyl or C₂₋₁₄ alkenyl;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, or sulfur;
each R^{x} is independently halogen, —CN, —OR, —SR, —C(O)R, —C(O)OR, or —OC(O)OR;
n is an integer from 10-75, inclusive;
m is 0, 1, 2, 3, or 4; and
t is 0, 1, or 2.

In some embodiments, the PEGylated lipid is a compound of formula PL-II′:

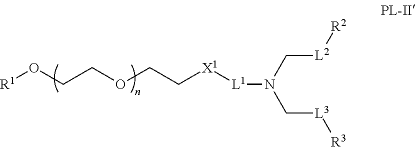

or a pharmaceutically acceptable salt thereof, wherein:
X¹ is —N(H)—, —N(C₁₋₆ alkyl)-, —C₁₋₆ aliphatic-N(H)—, —C₁₋₆ aliphatic-N(C₁₋₆ alkyl)—, —O— or —C₁₋₆ aliphatic-O—;
L¹ is —C(O)(C₁₋₆ aliphatic)C(O)—, —C(O)(C₁₋₆ aliphatic)-, or —C(O)—;
L² and L³ are a covalent bond or C₁₋₆ alkylene wherein one methylene unit of the C₁₋₆ alkylene is optionally replaced with —O—, —NR—, —S—, —S—S—, —S(O)—, —S(O)₂—, —C(O)—, —C(O)O—, —OC(O)—, —OC(O)O—, —OC(O)N(R)—, —N(R)C(O)O—, —C(O)N(R)—, —N(R)C(O)—, —N(R)C(O)N(R)—, —C(R⁶)═N—, or —C(R⁶)═N—O—;
R¹ is H, C₁₋₆ alkyl, —(C₁₋₆ alkyl)-N₃, —(C₁₋₆ alkyl)-SH, or C₃₋₈ alkynyl;
R² and R³ are independently straight or branched C₆₋₃₀ alkyl, straight or branched C₆₋₃₀ alkenyl, or straight or branched C₆₋₃₀ alkynyl; wherein 1, 2, or 3 methylene units are independently and optionally replaced by a saturated or partially unsaturated C₃₋₆ carbocyclic ring or phenylene; wherein the alkyl, alkenyl, and alkynyl and any carbocyclic ring or phenylene is substituted with m instances of R^{x};
R⁶ is C₁₋₆ alkyl or C₂₋₁₄ alkenyl;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, or sulfur;
each R^{x} is independently halogen, —CN, —OR, —SR, —C(O)R, —C(O)OR, or OC(O)OR;
n is an integer from 10-75, inclusive; and
m is 0, 1,2, 3, or 4.

In some embodiments, the PEGylated lipid compound is one of those shown in Table (VI), or a pharmaceutically acceptable salt thereof.

TABLE VI
Exemplary PEGylated Compounds

| Com- | |
|---|---|
| pound | |
| No. | Structure |
| PL- 1 | 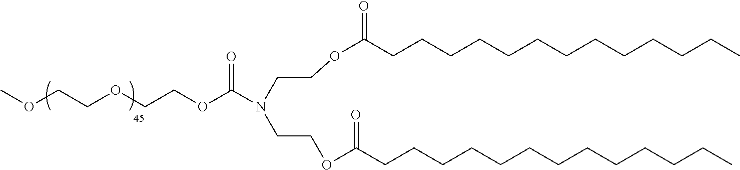 |
| PL- 1A | 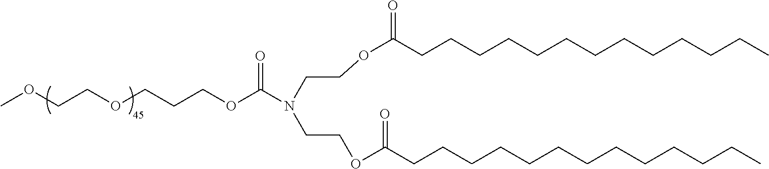 |
| PL- 2 | 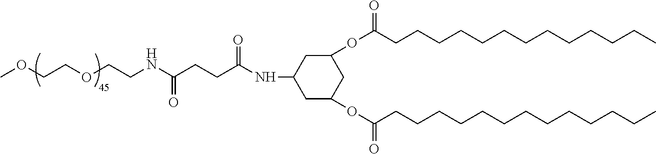 |
| PL- 2A | 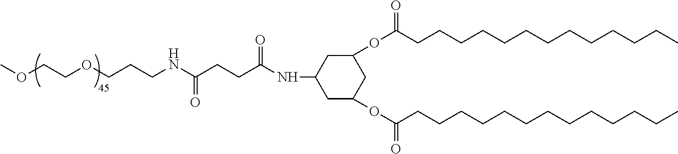 |
| PL- 3 | 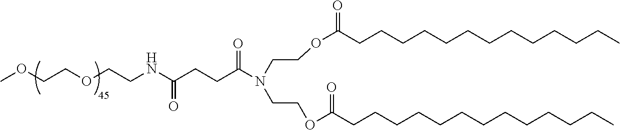 |
| PL- 3A | 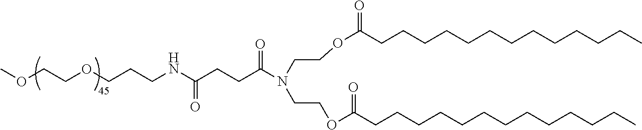 |
| PL- 4 | 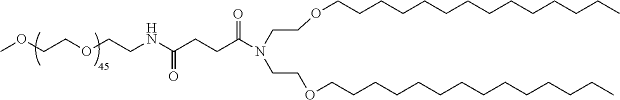 |
| PL- 4A | 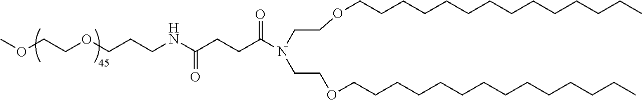 |
| PL- 5 | 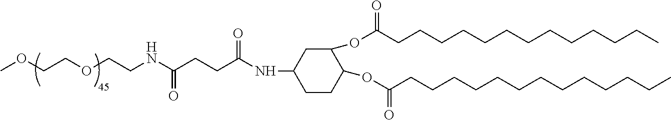 |
| PL- 5A | 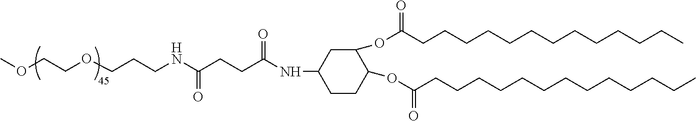 |
| PL- 6 | 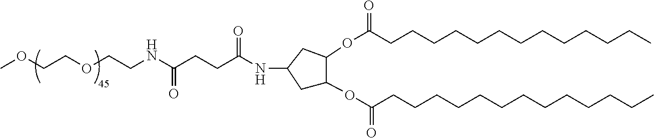 |
| PL- 6A | 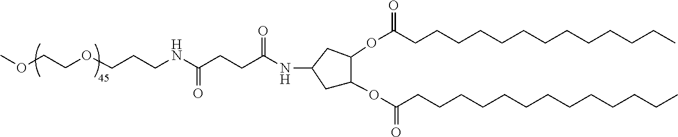 |
| PL- 7 | 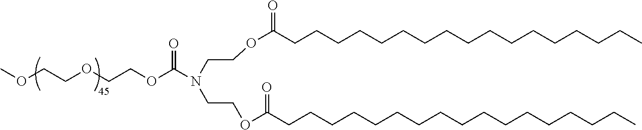 |
| PL- 7A | 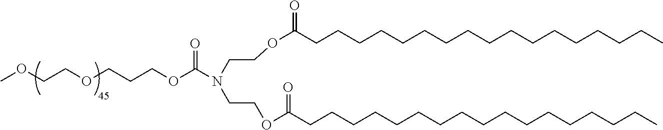 |
| PL- 8 | 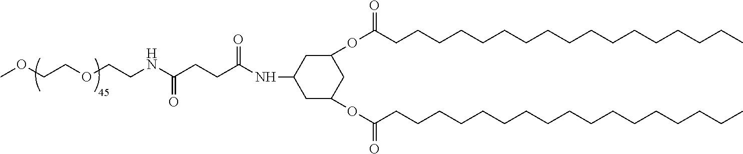 |
| PL- 8A | 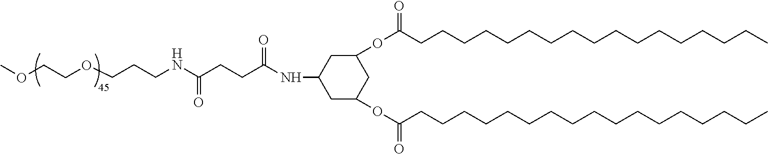 |
| PL- 9 | 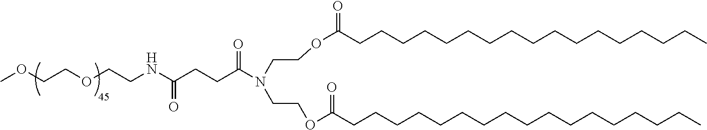 |
| PL- 9A | 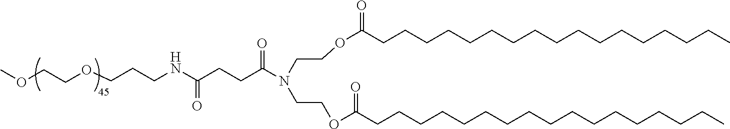 |
| PL- 10 | 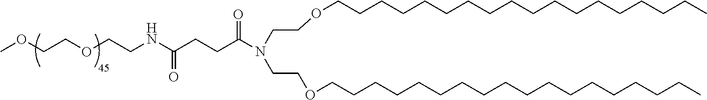 |
| PL- 10A | 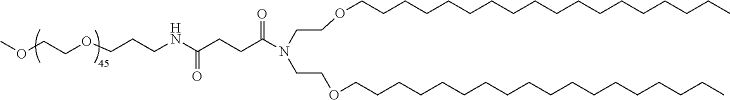 |
| PL- 11 | 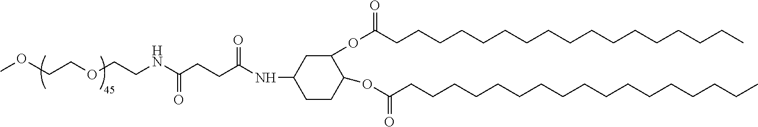 |
| PL- 11A | 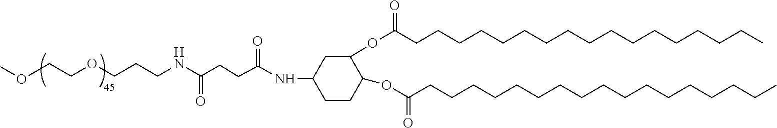 |
| PL- 12 | 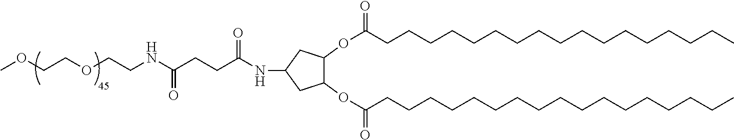 |
| PL- 12A | 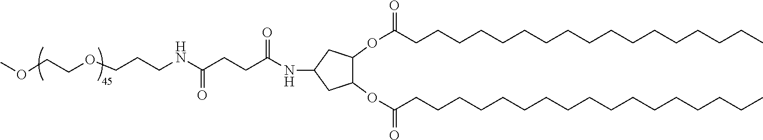 |
| PL- 12B | 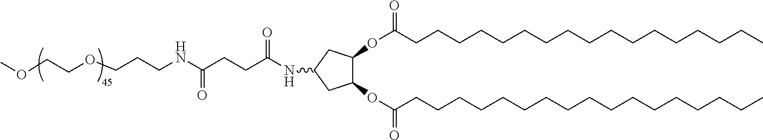 |
| PL- 13 | 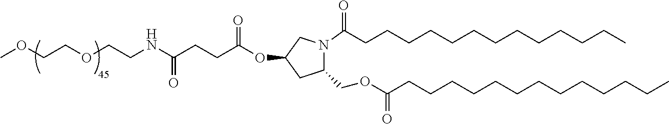 |
| PL- 13A | 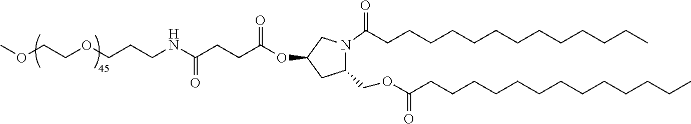 |
| PL- 14 | 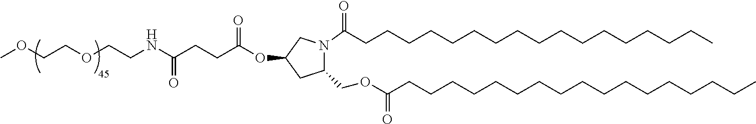 |
| PL- 14A | 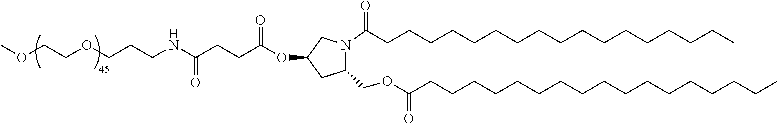 |
| PL- 15 | 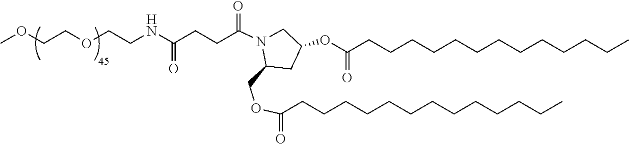 |
| PL- 15A | 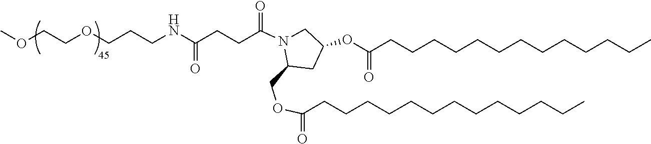 |
| PL- 16 | 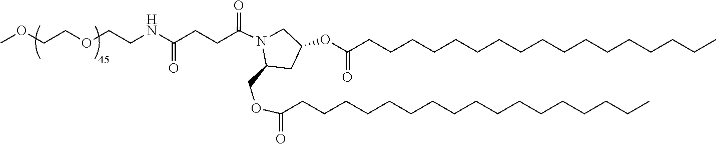 |
| PL- 16A | 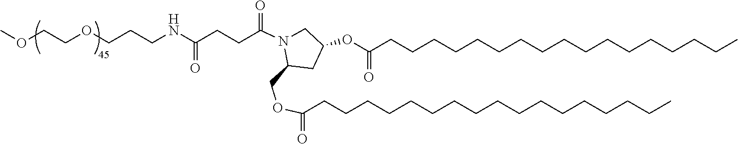 |

In some embodiments, the LNP comprises a PEGylated lipid substitute iii place of the PEGylated lipid. All embodiments disclosed herein that contemplate a PEGylated lipid should be understood to also apply to PEGylated lipid substitutes. In some embodiments, the LNP comprises a polysarcosine-lipid conjugate, such as those disclosed in US 2022/0001025 A1, which is incorporated by reference herein in its entirety. In some embodiments the LNP comprises a polyoxazoline-lipid conjugate, such as those disclosed in US 2022/0249695 A1, which is incorporated by reference herein in its entirety.

### V. Phospholipids

In some embodiments, an LNP of the present disclosure comprises a phospholipid. In some embodiments, an LNP of the present disclosure comprises two or more phospholipids. Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium (S)-2-ammonio-3-((((R)-2-(oleoyloxy)-3-(stearoyloxy)propoxy)oxidophosphoryl)oxy)propanoate (L-a-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin. In some embodiments, an LNP includes DSPC. In certain embodiments, an LNP includes DOPE. In some embodiments, an LNP includes both DSPC and DOPE.

In some embodiments, the LNP comprises a phospholipid selected from 1-pentadecanoyl-2-oleoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-palmitoyl-2-oleoyl-glycero-3-phosphocholine, 1-palmitoyl-2-linoleoyl-sn-glycero-3-phosphocholine, 1-palmitoyl-2-arachidonoyl-sn-glycero-3-phosphocholine, 1-palmitoyl-2-docosahexaenoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-stearoyl-2-oleoyl-sn-glycero-3-phosphocholine, 1-stearoyl-2-linoleoyl-sn-glycero-3-phosphocholine, 1-stearoyl-2-arachidonoyl-sn-glycero-3-phosphocholine, 1-stearoyl-2-docosahexaenoyl-sn-glycero-3-phosphocholine, 1-oleoyl-2-myristoyl-sn-glycero-3-phosphocholine, 1-oleoyl-2-palmitoyl-sn-glycero-3-phosphocholine, 1-oleoyl-2-stearoyl-sn-glycero-3-phosphocholine, 1-palmitoyl-2-acetyl-sn-glycero-3-phosphocholine, 1,2-dioleoyl-sn-glycero-3-phospho-(1′-myo-inositol-3′,4′-bisphosphate), 1,2-dioleoyl-sn-glycero-3-phospho-(1′-myo-inositol-3′,5′-bisphosphate), 1,2-dioleoyl-sn-glycero-3-phospho-(1′-myo-inositol-4′,5′-bisphosphate), 1,2-dioleoyl-sn-glycero-3-phospho-(1′-myo-inositol-3′,4′,5′-trisphosphate), 1,2-dioleoyl-sn-glycero-3-phospho-(1′-myo-inositol-3′-phosphate), 1,2-dioleoyl-sn-glycero-3-phospho-(1′-myo-inositol-4′-phosphate), 1,2-dioleoyl-sn-glycero-3-phospho-(1′-myo-inositol-5′-phosphate), 1,2-dioleoyl-sn-glycero-3-phospho-(1′-myo-inositol), 1,2-dioleoyl-sn-glycero-3-phospho-L-serine, and 1-(8Z-octadecenoyl)-2-palmitoyl-sn-glycero-3-phosphocholine.

In some embodiments, the LNP comprises a phospholipid selected from DSPS (Distearoylphosphatidylserine), DSPG (1,2-distearoyl-sn-glycero-3-phospho-(1′-rac-glycerol)), DSPA (1,2-Distearoyl-sn-glycero-3-phosphate), diPhyPC (1,2-diphytanoyl-sn-glycero-3-phosphocholine), diPhy-diether-PC (1,2-di-O-phytanyl-sn-glycero-3-phosphocholine), diPhyPE (1,2-diphytanoyl-sn-glycero-3-phosphoethanolamine), diPhy-diether-PE (1,2-di-O-phytanyl-sn-glycero-3-phosphoethanolamine), diPhyPS (1,2-diphytanoyl-sn-glycero-3-phospho-L-serine), diPhyPG (1,2-diphytanoyl-sn-glycero-3-phospho-(1′-rac-glycerol)), diPhyPA (1,2-diphytanoyl-sn-glycero-3-phosphate), Egg PA (L-a-phosphatidic acid), and Soy PA (L-a-phosphatidic acid).

In some embodiments, the LNP comprises a phospholipid selected from 18:1 (A9-Cis) PE (DOPE), 18:0-18:1 PE (SOPE), C₁₆-18:1 PE, 16:0-18:1 PE (POPE), 18:1 BMP (S,R), 18:0-18:1 PC (SOPC), 16:0-18:1 PC (POPC), 4ME 16:0 Diether PE (4Me), 18:1 (A9-Trans) PE (DEPE), 16:1 PE (DPPE), and CL. In certain embodiments, the LNP comprises a phospholipid described or disclosed in Alvarez-Benedicto, et al. (Biomater. Sci., 2022, 10, 549) and Li, et al. (Asian Journal of Pharmaceutical Sciences, 2015, 10, 81-98).

In certain embodiments, the phospholipid is a sphingoid lipid or sphingolipid, such as, but not limited to sphingomyelin. As used herein, the terms “sphingoid lipid” and “sphingolipid” are meant to refer to a class of lipids containing a backbone comprising a sphingoid base. An exemplary sphingoid base is sphingosine. In certain embodiments, the LNP comprises a sphingolipid selected from Egg Sphingomyelin (Egg SM/ESM/(2S,3R,E)-3-hydroxy-2-palmitamidooctadec-4-en-1-yl (2-(trimethylammonio)ethyl) phosphate), Brain or Porcine Sphingomyelin (Brain SM/(2S,3R,E)-3-hydroxy-2-stearamidooctadec-4-en-1-yl (2-(trimethylammonio)ethyl) phosphate), Milk or Bovine Sphingomyelin (Milk SM/(2S,3R,E)-3-hydroxy-2-tricosanamidooctadec-4-en-1-yl (2-(trimethylammonio)ethyl) phosphate), 28:0 SM (N-octacosanoyl-D-erythro-sphingosylphosphorylcholine), 14:0 SM (N-myristoyl-D-erythro-sphingosylphosphorylcholine), 16:1 SM (N-palmitoleoyl-D-erythro-sphingosylphosphorylcholine), 12:0 Dihydro SM (N-lauroyl-D-erythro-sphinganylphosphorylcholine), Lyso SM (Sphingosylphosphorylcholine), Lyso SM (Sphingosylphosphorylcholine), Lyso SM (dihydro) (Sphinganine Phosphorylcholine), 24:1 SM (N-nervonoyl-D-erythro-sphingosylphosphorylcholine), 24:0 SM (N-lignoceroyl-D-erythro-sphingosylphosphorylcholine), 18:1 SM (N-oleoyl-D-erythro-sphingosylphosphorylcholine), 18:0 SM (N-stearoyl-D-erythro-sphingosylphosphorylcholine), 17:0 SM (N-heptadecanoyl-D-erythro-sphingosylphosphorylcholine), 16:0 SM (N-palmitoyl-D-erythro-sphingosylphosphorylcholine), 12:0 SM (N-lauroyl-D-erythro-sphingosylphosphorylcholine), 06:0 SM (N-hexanoyl-D-erythro-sphingosylphosphorylcholine), 02:0 SM (N-acetyl-D-erythro-sphingosylphosphorylcholine), 3-O-methyl Lyso SM (3-O-methyl-spingosylphosphorylcholine), 3-O-methyl-N-methyl Lyso SM (3-O-methyl-N-methyl-spingosylphosphorylcholine), and 3-N-methyl Lyso SM (3-N-methyl-spingosylphosphorylcholine).

In some embodiments, the LNP comprises a phospholipid comprising at least one constrained tail, such as those described by Gan, et al. (Bioeng Transl Med. 2020 September; 5(3): e10161.). In certain embodiments, the phospholipid is one selected from:

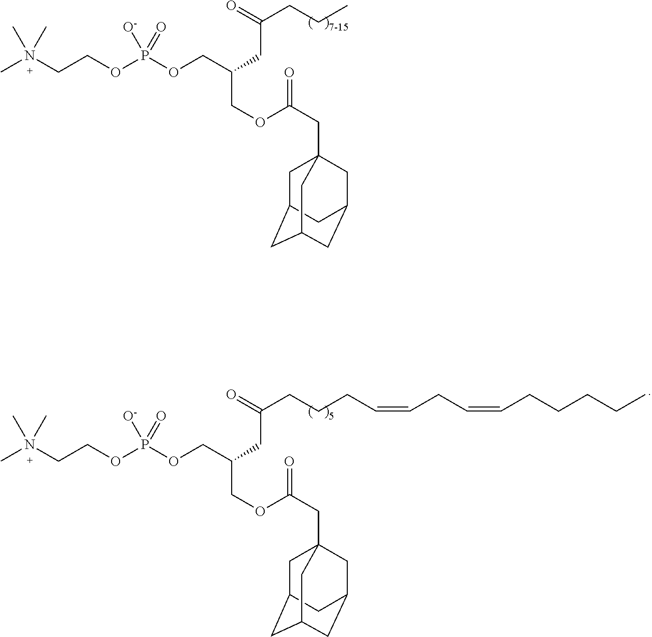

In some embodiments, the LNP comprises a phospholipid comprising a ceramide analogue having a triazole linkage, such as those described by Kim et al., Bioorg.

Med. Chem. Lett., 17(16), 2007, 4584-4587.

In some embodiments, the LNP comprises a phospholipid disclosed in WO 2023/141470, which is incorporated by reference herein, in its entirety. In certain embodiments, the phospholipid is

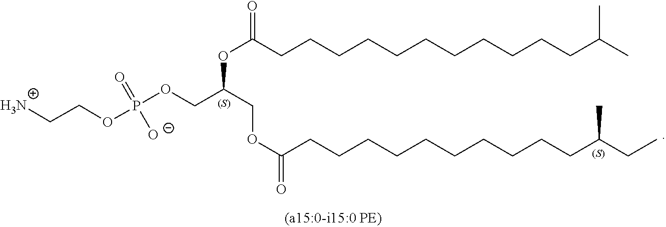

In some embodiments, the LNP comprises a phospholipid disclosed in WO 2022/040641, which is incorporated by reference herein, in its entirety.

In some embodiments, a phospholipid tail may be modified in order to promote endosomal escape as described in U.S. Application Publication 2021/0121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, phospholipids disclosed in US 2020/0121809 have the following structure:

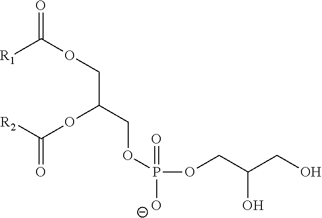

wherein R1 and R2 are each independently a branched or straight, saturated or unsaturated carbon chain (e.g., alkyl, alkenyl, alkynyl).
vi. Targeting moieties

In some embodiments, the lipid nanoparticle further comprises a targeting moiety. The targeting moiety may be an antibody or a fragment thereof. The targeting moiety may be capable of binding to a target antigen. In certain embodiments, the lipid nanoparticle comprises more than one targeting moiety. In certain embodiments, the lipid nanoparticle comprises more than one targeting moiety, wherein the targeting moieties target at least two different receptors, and in some embodiments, the at least two different receptors are prevalent on different types of cells or tissues.

In some embodiments, the pharmaceutical composition comprises a targeting moiety that is operably connected to a lipid nanoparticle. In some embodiments, the targeting moiety is capable of binding to a target antigen. In some embodiments, the target antigen is expressed in a target organ. In some embodiments, the target antigen is expressed more in the target organ than it is in the liver.

In some embodiments, the targeting moiety is an antibody as described in WO2016189532A1, which is incorporated herein by reference. For example, in some embodiments, the targeted particles are conjugated to a specific anti-CD38 monoclonal antibody (mAb), which allows specific delivery of the siRNAs encapsulated within the particles at a greater percentage to B-cell lymphocytes malignancies (such as MCL) than to other subtypes of leukocytes.

In some embodiments, the targeting moiety is a small molecule. In some embodiments, the small molecule binds to an ectoenzyme on an immune cell, wherein the ectoenzyme is selected from the group consisting of CD38, CD73, adenosine 2a receptor, and adenosine 2b receptor. In some embodiments, the small molecule is mannose, a lectin, acivicin, biotin, or digoxigenin.

In some embodiments, the targeting moiety targets a receptor selected from CD20, CCR7, CD3, CD4, CD5, CD8, CD16, CD19, CD20, CD21, CD22, CD25, CD28, CD35, CD40, CD45RA, CD45RO, CD52, CD62L, CD80, CD95, CD127, and CD137. In some embodiments, the targeting moiety targets a receptor selected from CD1, CD2, CD3, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, 0X40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7. In some embodiments, the targeting moiety targets a receptor selected from CD2, CD3, CD5 and CD7. In some embodiments, the targeting moiety targets a receptor selected from CD2, CD3, CD5, CD7, CD8, CD4, beta 7 integrin, beta 2 integrin, and C1q. In some embodiments, the targeting moiety targets CD 117. In some embodiments, the targeting moiety targets CD90. In some embodiments, the targeting moiety targets a receptor selected from a mannose receptor, CD206 and C1q. In some embodiments, the targeting moiety is selected from T-cell receptor motif antibodies, T-cell α chain antibodies, T-cell β chain antibodies, T-cell γ chain antibodies, T-cell δ chain antibodies, CCR7 antibodies, CD3 antibodies, CD4 antibodies, CD5 antibodies, CD7 antibodies, CD8 antibodies, CD11b antibodies, CD11c antibodies, CD16 antibodies, CD19 antibodies, CD20 antibodies, CD21 antibodies, CD22 antibodies, CD25 antibodies, CD28 antibodies, CD34 antibodies, CD35 antibodies, CD40 antibodies, CD45RA antibodies, CD45RO antibodies, CD52 antibodies, CD56 antibodies, CD62L antibodies, CD68 antibodies, CD80 antibodies, CD95 antibodies, CD117 antibodies, CD127 antibodies, CD133 antibodies, CD137 (4-1BB) antibodies, CD163 antibodies, F4/80 antibodies, IL-4Ra antibodies, Sca-1 antibodies, CTLA-4 antibodies, GITR antibodies GARP antibodies, LAP antibodies, granzyme B antibodies, LFA-1 antibodies, transferrin receptor antibodies, and fragments thereof. In certain embodiments, the targeting moiety is any one described or contemplated in US20230312713A1, US20230203538A1, US20230320995A1, US20160145348, and US20110038941, each of which is incorporated by reference herein in its entirety.

In some embodiments, the lipid nanoparticles may be targeted when conjugated/attached/associated with a targeting moiety such as an antibody.

vii. Zwitterionic Amino Lipids

In some embodiments, an LNP comprises a zwitterionic lipid. In some embodiments, an LNP comprising a zwitterionic lipid does not comprise a phospholipid.

Zwitterionic amino lipids have been shown to be able to self-assemble into LNPs without phospholipids to load, stabilize, and release mRNAs intracellularly as described in U.S. Patent Application 20210121411, which is incorporated herein by reference in its entirety. Zwitterionic, ionizable cationic and permanently cationic helper lipids enable tissue-selective mRNA delivery and CRISPR-Cas9 gene editing in spleen, liver and lungs as described in Liu et al., Membrane-destabilizing ionizable phospholipids for organ-selective mRNA delivery and CRISPR-Cas gene editing, Nat Mater. (2021), which is incorporated herein by reference in its entirety.

The zwitterionic lipids may have head groups containing a cationic amine and an anionic carboxylate as described in Walsh et al., Synthesis, Characterization and Evaluation of Ionizable Lysine-Based Lipids for siRNA Delivery, Bioconjug Chem. (2013), which is incorporated herein by reference in its entirety. Ionizable lysine-based lipids containing a lysine head group linked to a long-chain dialkylamine through an amide linkage at the lysine α-amine may reduce immunogenicity as described in Walsh et al., Synthesis, Characterization and Evaluation of Ionizable Lysine-Based Lipids for siRNA Delivery, Bioconjug Chem. (2013).

viii. Additional Lipid Components

In some embodiments, the LNP compositions of the present disclosure further comprise one or more additional lipid components capable of influencing the tropism of the LNP. In some embodiments, the LNP further comprises at least one lipid selected from DDAB, EPC, 14PA, 18BMP, DODAP, DOTAP, and C12-200 (see Cheng, et al. Nat Nanotechnol. 2020 April; 15(4): 313-320.; Dillard, et al. PNAS 2021 Vol. 118 No. 52.).

In some embodiments, the LNP compositions of the present disclosure comprise, or further comprise one or more lipids selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Nα-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-a-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide](MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g.1,2-dioleoyl-sn-glycero-3-phosphoethanolamine-N-[4-(p-maleimidophenyl)but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol(PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.

### III. LNP Payload

In various aspects, the LNPs described herein may be used to deliver a payload of interest to a biological target, e.g., to a cell or a bodily tissue. The term “payload” refers to an active substance, such as a small molecule, polypeptide, peptide, carbohydrate, or nucleic acid molecule, and includes, without limitation, mRNA molecules (including linear and circular mRNA) which are encapsulated within the LNPs described herein. In various embodiments, the payload is an RNA molecule, which may be linear or circular and may comprise one or more functional nucleotide sequences of interest, which may include, but are not limited to coding and non-coding nucleotide sequences. In various embodiments, the non-coding nucleotide sequences may comprise regulatory elements that influence RNA post-transcriptional processing, nuclear translation control sequences, and sequences which encode one or more biological products of interest, e.g., a therapeutic protein or nucleobase editing system, among other sequence elements that may impact the functioning of the RNA or its encoded products. As used herein, the term “coding region of interest” or “product coding region” or the like may be used to refer to the encoded one or more biological products of interest. Equivalently, a product coding region may be referred to as a “product expression sequence.”

### A. Nucleic Acid Payloads

In various embodiments, the LNP compositions described herein can be used to deliver a nucleic acid or polynucleotide payload, e.g., a linear or circular mRNA.

In some embodiments, a LNP is capable of delivering a polynucleotide to a target cell, tissue, or organ. A polynucleotide, in its broadest sense of the term, includes any compound and/or substance that is or can be incorporated into an oligonucleotide chain.

Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. RNAs useful in the compositions and methods described herein can be selected from the group consisting of but are not limited to, shortimers, antagomirs, antisense, ribozymes, short interfering RNA (siRNA), asymmetrical interfering RNA (aiRNA), microRNA (miRNA), Dicer substrate RNA (dsRNA), short hairpin RNA (shRNA), transfer RNA (tRNA), messenger RNA (mRNA), and mixtures thereof. In some embodiments, a polynucleotide is mRNA. In some embodiments, a polynucleotide is circular RNA. In some embodiments, a polynucleotide encodes a protein, e.g., a nucleobase editing enzyme. A polynucleotide may encode any polypeptide of interest, including any naturally or non-naturally occurring or otherwise modified polypeptide. A polypeptide may be of any size and may have any secondary structure or activity. In some embodiments, a polypeptide encoded by an mRNA may have a therapeutic effect when expressed in a cell.

In other embodiments, a polynucleotide is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, a polynucleotide is an shRNA or a vector or plasmid encoding the same. An shRNA may be produced inside a target cell upon delivery of an appropriate construct to the nucleus. Constructs and mechanisms relating to shRNA are well known in the relevant arts.

A polynucleotide may include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3-UTR), at least one 5′-cap region, and a 3-stabilizing region. In some embodiments, a polynucleotide further includes a poly-A region or a Kozak sequence (e.g., in the 5′-UTR). In some cases, polynucleotides may contain one or more intronic nucleotide sequences capable of being excised from the polynucleotide. In some embodiments, a polynucleotide (e.g., an mRNA) may include a 5′cap structure, a chain terminating nucleotide, a stem loop, a polyA sequence, and/or a polyadenylation signal. Any one of the regions of a nucleic acid may include one or more alternative components (e.g., an alternative nucleoside). For example, the 3-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyu ridine), a 1-substituted pseudouridine (e.g., 1-methyl pseudouridine or 1-ethyl-pseudouridine), and/or a 5-substituted cytidine (e.g., 5-methyl-cytidine). In some embodiments, a polynucleotide contains only naturally occurring nucleosides.

In some cases, a polynucleotide is greater than 30 nucleotides in length. In another embodiment, the poly nucleotide molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 50 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In some embodiments, a polynucleotide molecule, formula, composition or method associated therewith comprises one or more polynucleotides comprising features as described in WO2002/098443, WO2003/051401, WO2008/052770, WO2009/127230, WO2006/122828, WO2008/083949, WO2010/088927, WO2010/037539, WO2004/004743, WO2005/016376, WO2006/024518, WO2007/095,976, WO2008/014979, WO2008/077592, WO2009/030481, WO2009/095226, WO2011/069586, WO2011/026641, WO2011/144358, WO2012/019780, WO2012/013326, WO2012/089338, WO2012/113513, WO2012/116811, WO2012/116810, WO2013/113502, WO2013/113501, WO2013/113736, WO2013/143698, WO2013/143699, WO2013/143700, WO2013/120626, WO2013/120627, WO2013/120628, WO2013/120629, WO2013/174409, WO2014/127917, WO2015/024669, WO2015/024668, WO2015/024667, WO2015/024665, WO2015/024666, WO2015/024664, WO2015/101415, WO2015/101414, WO2015/024667, WO2015/062738, WO2015/101416, all of which are incorporated by reference herein.

In some embodiments, a polynucleotide comprises one or more microRNA binding sites. In some embodiments, a microRNA binding site is recognized by a microRNA in a non-target organ. In some embodiments, a microRNA binding site is recognized by a microRNA in the liver. In some embodiments, a microRNA binding site is recognized by a microRNA in hepatic cells.

B. Linear mRNA Payloads

In various embodiments, the LNP-based nucleobase editing systems, RNA therapeutics and pharmaceutical compositions thereof described herein can be used to deliver an RNA payload that is a linear mRNA molecule.

Ribonucleic acid (RNA) is a molecule that is made up of nucleotides, which are ribose sugars attached to nitrogenous bases and phosphate groups. The nitrogenous bases include adenine (A), guanine (G), uracil (U), and cytosine (C). Generally, RNA mostly exists in the single-stranded form but can also exists double-stranded in certain circumstances. The length, form and structure of RNA is diverse depending on the purpose of the RNA. For example, the length of an RNA can vary from a short sequence (e.g., siRNA) to a long sequences (e.g., lncRNA), can be linear (e.g., mRNA) or circular (e.g., oRNA), and can either be a coding (e.g., mRNA) or a non-coding (e.g., lncRNA) sequence.

In various embodiments, the LNP-based nucleobase editing systems, RNA therapeutics and pharmaceutical compositions thereof described herein can be used to deliver a mRNA payload that is a linear mRNA molecule. In embodiments, the mRNA payload may comprise one or more nucleotide sequences that encode a product of interest, such as, but not limited to a component of a gene editing system (e.g. an endonuclease, a prime editor, etc.) and/or a therapeutic protein.

In some embodiments, the RNA payload may be a linear mRNA. As used herein, the term “messenger RNA” (mRNA) refers to any polynucleotide which encodes a protein of interest and which is capable of being translated to produce the encoded protein of interest in vitro, in vivo, in situ or ex vivo.

Generally, a mRNA molecule comprises at least a coding region, a 5′ untranslated region (UTR), a 3′ UTR, a 5′ cap and a poly-A tail. In some aspects, one or more structural and/or chemical modifications or alterations may be included in the RNA which can reduce the innate immune response of a cell in which the mRNA is introduced. As used herein, a “structural” feature or modification is one in which two or more linked nucleotides are inserted, deleted, duplicated, inverted or randomized in a nucleic acid without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”.

Generally, a coding region of interest in an mRNA used herein may encode a dipeptide, a tripeptide, a tetrapeptide, a pentapeptide, a hexapeptide, a heptapeptide, an octapeptide, a nonapeptide, or a decapeptide. In another embodiment, the mRNA may encode a peptide of 2-30 amino acids, e.g. 5-30, 10-30, 2-25, 5-25, 10-25, or 10-20 amino acids. The mRNA may encode a peptide of at least 10, 11, 12, 13, 14, 15, 17, 20, 25, 26, 27, 28, 29, 30, 31, 32, 33, 34, 35, 36, 37, 38, 39, or 40 amino acids, or a peptide that is no longer than 10, 11, 12, 13, 14, 15, 17, 20, 25, 26, 27, 28, 29, 30, 31, 32, 33, 34, 35, 36, 37, 38, 39, or 40 amino acids.

Generally, the length of the region of the mRNA encoding a product of interest is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the mRNA has a total length that spans from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some embodiments, the region or regions flanking the region encoding the product of interest may range independently from 15-1,000 nucleotides in length (e.g., greater than 30, 40, 45, 50, 55, 60, 70, 80, 90, 100, 120, 140, 160,180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, the mRNA comprises a tailing sequence which can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the mRNA comprises a capping sequence which comprises a single cap or a series of nucleotides forming the cap. The capping sequence may be from 1 to 10, e.g. 2-9, 3-8, 4-7, 1-5, 5-10, or at least 2, or 10 or fewer nucleotides in length. In some embodiments, the caping sequence is absent.

In some embodiments, the mRNA comprises a region comprising a start codon. The region comprising the start codon may range from 3 to 40, e.g., 5-30, 10-20, 15, or at least 4, or 30 or fewer nucleotides in length.

In some embodiments, the mRNA comprises a region comprising a stop codon. The region comprising the stop codon may range from 3 to 40, e.g., 5-30, 10-20, 15, or at least 4, or 30 or fewer nucleotides in length.

In some embodiments, the mRNA comprises a region comprising a restriction sequence. The region comprising the restriction sequence may range from 3 to 40, e.g., 5-30, 10-20, 15, or at least 4, or 30 or fewer nucleotides in length.

### Untranslated Regions (UTRs)

In various embodiments, the mRNA payloads of the LNP-based nucleobase editing systems, RNA therapeutics and pharmaceutical compositions thereof described herein, may comprise at least one untranslated region (UTR) which flanks the region encoding the product of interest and/or is incorporated within the mRNA molecule. UTRs are transcribed by not translated. The mRNA payloads can include 5′ UTR sequences and 3′ UTR sequences, as well as internal UTRs.

The RNA payloads of the present disclosure may comprise one or more regions or parts which act or function as an untranslated region. Where nucleic acids are designed to encode at least one polypeptide of interest, the nucleic acid may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the RNA payload molecules (e.g., linear and circular mRNA molecules) of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

In various embodiments, the mRNA payloads of the LNP-based nucleobase editing systems, RNA therapeutics and pharmaceutical compositions thereof described herein, may comprise at least one UTR that may be selected from any UTR sequence listed in Tables 19 or 20 of U.S. Pat. No. 10,709,779, which is incorporated herein by reference.

### 5′ UTR Regions

In various embodiments, the mRNA payloads of the LNP-based nucleobase editing systems, RNA therapeutics and pharmaceutical compositions thereof described herein, may comprise at least one 5′ UTR.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding. 5′ UTR sequences are also known to be important for ribosome recruitment to the mRNA and have been reported to play a role in translation (Hinnebusch A, et al., (2016) Science, 352:6292: 1413-6). In addition, 5′ UTR sequences may confer increased half-life, increased expression and/or increased activity of a polypeptide encoded by the RNA payload described herein.

In various embodiments, the RNA payload constructs contemplated herein may include 5′UTRs that are found in nature and those that are not. For example, the 5′UTRs can be synthetic and/or can be altered in sequence with respect to a naturally occurring 5′UTR. Such altered 5′UTRs can include one or more modifications relative to a naturally occurring 5′UTR, such as, for example, an insertion, deletion, or an altered sequence, or the substitution of one or more nucleotide analogs in place of a naturally occurring nucleotide.

The 5′ UTR starts at the transcription start site and continues to the start codon but does not include the start codon; whereas, the 3′UTR starts immediately following the stop codon and continues until the transcriptional termination signal. While not wishing to be bound by theory, the UTRs may have a regulatory role in terms of translation and stability of the nucleic acid.

Natural 5′ UTRs usually include features which have a role in translation initiation as they tend to include Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

In an embodiment, the 5′ UTR comprises a sequence provided in Table X or a sequence having at least 80%, 85%, 90%, 95%, 96%, 97%, 98%, 99% or 100% sequence identity to a 5′ UTR sequence provided in Table X, or a variant or a fragment thereof (e.g., a fragment that lacks the first one, two, three, four, five, or six nucleotides of the 5′ UTR sequence provided in Table X). In an embodiment, the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

TABLE X
Exemplary nucleotide sequences of 5′ UTRs

| | Sequence |
|---|---|
| 5′ UTR Nucleotide Sequence | Identifier |
| ggaaaucgca aaauuugcuc uucgcguuag auuucuuuua guuuucucgc | SEQ ID NO: 1 |
| aacuagcaag cuuuuuguuc ucgccgccgc c | |
| ggaaaucgca aaauuuucuu uucgcguuag auuucuuuua guuuucuuuc | SEQ ID NO: 2 |
| aacuagcaag cuuuuuguuc ucgccgccgc c | |
| ggaaaucgca aaauuuuugc ucuuuuucgc guuagauuuc uuuuaguuuu | SEQ ID NO: 3 |
| cuykcaacua gcaagcuuuu uguucucgcc rcc | |
| ggaaaucccc acaaccgccu cauauccagg cucaagaaua gagcucagug | SEQ ID NO: 4 |
| uuuuguuguu uaaucauucc gacguguuuu gcgauauucg cgcaaagcag | |
| ccagucgcgc gcuugcuuuu aaguagaguu guuuuuccac ccguuugcca | |
| ggcaucuuua auuuaacaua uuuuauuuuu ucaggcuaac cuacgccgcc | |
| acc | |
| ggaaauaaga gagaaaagaa gaguaagaag aaauauaaga ucucccugag | SEQ ID NO: 5 |
| cuucagggag ccccggcgcc gccacc | |
| ggaaaccccc cacccccgua agagagaaaa gaagaguaag aagaaauaua | SEQ ID NO: 6 |
| agaucucccu gagcuucagg gagccccggc gccgccacc | |
| ggagaacuuc cgcuuccguu ggcgcaagcg cuuucauuuu uucugcuacc | SEQ ID NO: 7 |
| gugacuaag | |
| ggaaauaaga gagaaaagaa gaguaagaag aaauauaaga gccacc | SEQ ID NO: 8 |
| ggaaauaaga gagaaaagaa gaguaagaag aaauauaaga ccccggcgcc gccacc | SEQ ID NO: 9 |
| ggaaacuuua uuuaguguua cuuuauuuuc uguuuauuug uguuucuuca | SEQ ID NO: 10 |
| guggguuugu ucuaauuucc uuggccgcc | |
| ggaaaaucug uauuagguug gcguguucuu uggucgguug uuaguauugu | SEQ ID NO: 11 |
| uguugauucg uuuguggucg guugccgcc | |
| ggaaaauuau uaacaucuug guauucucga uaaccauucg uuggauuuua | SEQ ID NO: 12 |
| uuguauucgu aguuuggguu ccugccgcc | |
| ggaaauuauu auuauuucua gcuacaauuu aucauuguau uuuuuagcu | SEQ ID NO: 13 |
| auucaucauu auuuacuugg ugaucaaca | |
| ggaaauaggu uguuaaccaa guucaagccu aauaagcuug gauucuggug | SEQ ID NO: 14 |
| acuugcuuca ccguuggcgg gcaccgauc | |
| ggaaaucgua gagagucgua cuuaguacau aucgacuauc gguggacacc | SEQ ID NO: 15 |
| aucaagauua uaaaccaggc caga | |
| ggaaacccgc ccaagcgacc ccaacauauc agcaguugcc caaucccaac | SEQ ID NO: 16 |
| ucccaacaca auccccaagc aacgccgcc | |
| ggaaagcgau ugaaggcguc uuuucaacua cucgauuaag guuggguauc | SEQ ID NO: 17 |
| gucgugggac uuggaaauuu guuguuucc | |
| ggaaacuaau cgaaauaaaa gagccccgua cucuuuuauu ucuauuaggu | SEQ ID NO: 18 |
| uaggagccuu agcauuugua ucuuaggua | |
| ggaaauguga uuuccagcaa cuucuuuuga auauauugaa uuccuaauuc | SEQ ID NO: 19 |
| aaagcgaaca aaucuacaag ccauauacc | |
| ggaaaucgua gagagucgua cuuacguggu cgccauugca uagcgcgcga | SEQ ID NO: 20 |
| aagcaacagg aacaagaacg cgcc | |
| ggaaaucgua gagagucgua cuuagaauaa acagagucgg gucgacuugu | SEQ ID NO: 21 |
| cucugauacu acgacgucac aauc | |
| ggaaaauuug ccuucggagu ugcguauccu gaacugccca gccuccugau | SEQ ID NO: 22 |
| auacaacugu uccgcuuauu cgggccgcc | |
| ggaaaucuga gcaggaaucc uuugugcauu gaagacuuua gauuccucuc | SEQ ID NO: 23 |
| ugcgguagac gugcacuuau aaguauuug | |
| ggaaagcgau ugaaggcguc uuuucaacua cucgauuaag guuggguauc | SEQ ID NO: 24 |
| gucgugggac uuggaaauuu guugccacc | |
| ggaaaauuuu agccuggaac guuagauaac uguccuguug ucuuuauaua | SEQ ID NO: 25 |
| cuuggucccc aaguaguuug ucuuccaaa | |
| ggaaauuuuu uuuugauauu auaagaguuu uuuuuugaua uuaagaaaau | SEQ ID NO: 26 |
| uuuuuuuuga uauuagaaga guaagaagaa auauaagacc ccggcgccgc cacc | |
| ggaaauaaga gagaaaagaa gaguaagaag aaauauaaga gccaaaaaaa aaaaacc | SEQ ID NO: 27 |
| ggaaaucucc cugagcuuca gggaguaaga gagaaaagaa gaguaagaag | SEQ ID NO: 28 |
| aaauauaaga ccccggcgcc gccacc | |

In some embodiments of the disclosure, a 5′ UTR is a heterologous UTR, i.e., is a UTR found in nature associated with a different mRNA. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived alpha-globin or beta-globin (e.g., U.S. Pat. Nos. 8,278,063 and 9,012,219), human cytochrome b-245 polypeptide, and hydroxysteroid (17b) dehydrogenase, and Tobacco etch virus. CMV immediate-early 1 (IE1) gene (see US20140206753 and WO2013/185069), the sequence GGGAUCCUACC (SEQ ID NO: 29) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738)), 5′ UTR element derived from the 5′UTR of an hydroxysteroid (17-P) dehydrogenase 4 gene (HSD17B4) (WO2015024667), or a 5′ UTR element derived from the 5′ UTR of ATP5A1 (WO2015024667) can be used. In one embodiment, an internal ribosome entry site (IRES) is used as a substitute for a 5′ UTR.

In some embodiments, a 5′ UTR of the present disclosure comprises SEQ ID NO: 30 (GGGAAAUAAG AGAGAAAAGA AGAGUAAGAA GAAAUAUAAG AGCCACC).

### 3′ UTR Regions

In various embodiments, the mRNA payloads of the LNP-based nucleobase editing systems, RNA therapeutics and pharmaceutical compositions thereof described herein, may comprise at least one 3′ UTR. 3′ UTRs may be heterologous or synthetic.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al., 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the mRNA payloads described herein. For example, one or more copies of an ARE can be introduced to make mRNA less stable and thereby curtail translation and decrease production of the resultant protein. Alternatively, AREs can be identified and removed or mutated to increase the intracellular stability and thus increase translation and production of the resultant protein.

In some embodiments, the introduction of features often expressed in genes of target organs the stability and protein production of the mRNA can be enhanced in a specific organ and/or tissue. As a non-limiting example, the feature can be a UTR. As another example, the feature can be introns or portions of introns sequences.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within an RNA payload construct. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the polypeptide coding region of interest in an mRNA payload may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety

It should be understood that any UTR from any gene may be incorporated into the regions of an RNA payload molecule (e.g., a linear mRNA). Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### 5′ Capping

In various embodiments, the mRNA payloads of the LNP-based nucleobase editing systems, RNA therapeutics and pharmaceutical compositions thereof described herein, may comprise a 5′ cap structure.

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to mRNA may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional modified guanosine nucleotides may be used such as a-methyl-phosphonate and seleno-phosphate nucleotides.

Additional modifications include, but are not limited to, 2′-0-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N₇ methyl group as well as a 3′-0-methyl group (i.e., N7,3′-0-dimethyl-guanosine-5′-triphosphate-5′-guanosine (m⁷G-3′mppp-G; which may equivalently be designated 3′ O-Me-m7G(5′)ppp(5′)G). The 3′-0 atom of the other, unmodified, guanine becomes linked to the 5′-terminal nucleotide of the capped nucleic acid molecule (e.g. an mRNA). The N7- and 3′-0-methlyated guanine provides the terminal moiety of the capped nucleic acid molecule (e.g. mRNA).

Another exemplary cap is mCAP, which is similar to ARCA but has a 2′-0-methyl group on guanosine (i.e., N7,2′-0-dimethyl-guanosine-5′-triphosphate-5′-guanosine, m⁷Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

mRNA may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-0-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-0-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5*)ppp(5*)N,pN2p (cap 0), 7mG(5*)ppp(5*)NlmpNp (cap 1), and 7mG(5*)-ppp(5′)NlmpN2mp (cap 2).

In some embodiments, the 5′ terminal caps may include endogenous caps or cap analogs.

In some embodiments, a 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### IRES Sequences

In various embodiments, the mRNA payloads of the LNP-based nucleobase editing systems, RNA therapeutics and pharmaceutical compositions thereof described herein, may comprise one or more IRES sequences.

In some embodiments, the mRNA may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA.

An mRNA that contains more than one functional ribosome binding site may encode several peptides or polypeptides that are translated independently by the ribosomes. Non-limiting examples of IRES sequences that can be used include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In some embodiments, the IRES is from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATiR, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAPI, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVBS, EVA71, CVA3, CVA12, EV24 or an aptamer to eIF4G.

### Poly-A Tails and 3′ Stabilizing Region

In various embodiments, the mRNA payloads of the LNP-based nucleobase editing systems, RNA therapeutics and pharmaceutical compositions thereof described herein, may comprise a poly-A tail.

During RNA processing, a long chain of adenine nucleotides (poly-A tail) may be added to a polynucleotide such as an mRNA molecules in order to increase stability. Immediately after transcription, the 3′ end of the transcript may be cleaved to free a 3′ hydroxyl. Then poly-A polymerase adds a chain of adenine nucleotides to the free 3′ hydroxyl end. The process, called polyadenylation, adds a poly-A tail of a certain length.

In some embodiments, the length of a poly-A tail is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides) and no more than about 50, 100, 200, 300, 400, 500, 600, 700, 800, 900, 1000, 2000, or 3000 nucleotides in length. In some embodiments, the mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall mRNA. This design may be based on the length of the region coding for a target of interest, the length of a particular feature or region (such as a flanking region), or based on the length of the ultimate product expressed from the mRNA.

In this context the poly-A tail may be 10, 20, 30, 40, 50, 60, 70, 80, 90, or 100% greater in length than the mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for poly-A binding protein may enhance expression.

Additionally, multiple distinct mRNA may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the mRNA are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail.

### Stop Codons

In various embodiments, the mRNA payloads of the LNP-based nucleobase editing systems, RNA therapeutics and pharmaceutical compositions thereof described herein, may comprise one or more translation stop codons. Translational stop codons, UAA, UAG, and UGA, are an important component of the genetic code and signal the termination of translation of an mRNA. During protein synthesis, stop codons interact with protein release factors and this interaction can modulate ribosomal activity thus having an impact translation (Tate W P, et al., (2018) Biochem Soc Trans, 46(6):1615-162).

A stop element as used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons.

In some embodiments, the mRNA may include one stop codon. In some embodiments, the mRNA may include two stop codons. In some embodiments, the mRNA may include three stop codons. In some embodiments, the mRNA may include at least one stop codon. In some embodiments, the mRNA may include at least two stop codons. In some embodiments, the mRNA may include at least three stop codons. As non-limiting examples, the stop codon may be selected from TGA, TAA and TAG.

In other embodiments, the stop codon may be selected from one or more of the following stop elements of Table Y:

TABLE Y
Additional stop elements

| Nucleotide sequence | |
|---|---|
| (5′ to 3′) | Sequence Identifier |
| UGAUAAUAG | |
| UAAUAGUAA | |
| UAAGUCUAA | |
| UAAAGCUAA | |
| UAAGUCUCC | |
| UAAGGCUAA | |
| UAAGCCCCUCCGGGG | SEQ ID NO: 31 |
| UAAAGCUCCCCGGGG | SEQ ID NO: 32 |
| UAAGCCCCU | |
| UAAAGCUCC | |
| UAAAGCUCC | |
| UAGGGUUAA | |
| UAAGCACCC | |
| UGAUAGUAA | |
| UAAAGCGCU | |

In some embodiments, the mRNA includes the stop codon TGA and one additional stop codon. In a further embodiment the addition stop codon may be TAA.

### MicroRNA Binding Sites and Other Regulatory Elements

In various embodiments, the mRNA payloads of the LNP-based nucleobase editing systems, RNA therapeutics and pharmaceutical compositions thereof described herein, may comprise one or more regulatory elements, including, but not limited to microRNA (miRNA) binding sites, structured mRNA sequences and/or motifs, artificial binding sites to bind to endogenous nucleic acid binding molecules, and combinations thereof.

### Chemically Unmodified Nucleotides

In some embodiments, the mRNA payloads of the LNP-based nucleobase editing systems, RNA therapeutics and pharmaceutical compositions thereof described herein are not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemically Modified Nucleotides

In some embodiments, the mRNA payloads of the LNP-based nucleobase editing systems, RNA therapeutics and pharmaceutical compositions thereof described herein comprise, in some embodiments, comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m¹ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (ψ) and 5-methyl-cytidine (m⁵C). In some embodiments, polynucleotides (e.g., RNA polynucleotides, such as mRNA polynucleotides) comprise 1-methyl-pseudouridine (m¹ψ). In some embodiments, polynucleotides (e.g., RNA polynucleotides, such as mRNA polynucleotides) comprise 1-methyl-pseudouridine (m¹ψ) and 5-methyl-cytidine (m⁵C). In some embodiments, polynucleotides (e.g., RNA polynucleotides, such as mRNA polynucleotides) comprise 2-thiouridine (s²U). In some embodiments, polynucleotides (e.g., RNA polynucleotides, such as mRNA polynucleotides) comprise 2-thiouridine and 5-methyl-cytidine (m⁵C). In some embodiments, polynucleotides (e.g., RNA polynucleotides, such as mRNA polynucleotides) comprise methoxy-uridine (mo⁵U). In some embodiments, polynucleotides (e.g., RNA polynucleotides, such as mRNA polynucleotides) comprise 5-methoxy-uridine (mo⁵U) and 5-methyl-cytidine (m⁵C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m⁵C). In some embodiments, polynucleotides (e.g., RNA polynucleotides, such as mRNA polynucleotides) comprise N6-methyl-adenosine (m⁶A). In some embodiments, polynucleotides (e.g., RNA polynucleotides, such as mRNA polynucleotides) comprise N6-methyl-adenosine (m⁶A) and 5-methyl-cytidine (mC).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m⁵C), meaning that all cytosine residues in the mRNA sequence are replaced with 5-methyl-cytidine (m⁵C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+CorA+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

C. Circular mRNA Payloads

In various embodiments, the LNP-based nucleobase editing systems, RNA therapeutics and pharmaceutical compositions thereof described herein can be used to deliver an RNA payload that is a circular mRNA molecule or “oRNA.” The circular mRNA molecule may encode a CROI, such as a nucleobase editing system, or therapeutic protein as described in this specification.

In some embodiments, the RNA payload is a circular RNA (oRNA). As used herein, the terms “oRNA” or “circular RNA” are used interchangeably and can refer to a RNA that forms a circular structure through covalent or non-covalent bonds.

Circular RNA described herein are polyribonucleotides that form a continuous structure through covalent or non-covalent bonds. Due to the circular structure, oRNAs have improved stability, increased half-life, reduced immunogenicity, and/or improved functionality (e.g., of a function described herein) compared to a corresponding linear RNA.

In some embodiments, an oRNA binds a target. In some embodiments, an oRNA binds a substrate. In some embodiments, an oRNA binds a target and binds a substrate of the target. In some embodiments, an oRNA binds a target and mediates modulation of a substrate of the target. In some embodiments, an oRNA brings together a target and its substrate to mediate modification of the substrate, e.g., post-translational modification. In some embodiments, an oRNA brings together a target and its substrate to mediate a cellular process (e.g., alters protein degradation or signal transduction) involving the substrate. In some embodiments, a target is a target protein and a substrate is a substrate protein.

In some embodiments, an oRNA comprises a conjugation moiety for binding to chemical compound. The conjugation moiety can be a modified polyribonucleotide. The chemical compound can be conjugated to the oRNA by the conjugation moiety. In some embodiments, the chemical compound binds to a target and mediates modulation of a substrate of the target. In some embodiments, an oRNA binds a substrate of a target and a chemical compound conjugated to the oRNA by the conjugation moiety binds the target to bring together the target and its substrate to mediate modification of the substrate, e.g., post-translational modification. In some embodiments, an oRNA binds a substrate of a target and a chemical compound conjugated to the oRNA by the conjugation moiety binds the target to bring together the target and its substrate to mediate modification of the substrate to mediate a cellular process (e.g., alters protein degradation or signal transduction) involving the substrate. In some embodiments, a target is a target protein and a substrate is a substrate protein.

In some embodiments, the oRNA may be non-immunogenic in a mammal (e.g., a human, non-human primate, rabbit, rat, and mouse).

In some embodiments, the oRNA may be capable of replicating or replicates in a cell from an aquaculture animal (e.g., fish, crabs, shrimp, oysters etc.), a mammalian cell, a cell from a pet or zoo animal (e.g., cats, dogs, lizards, birds, lions, tigers and bears etc.), a cell from a farm or working animal (e.g., horses, cows, pigs, chickens etc.), a human cell, cultured cells, primary cells or cell lines, stem cells, progenitor cells, differentiated cells, germ cells, cancer cells (e.g., tumorigenic, metastatic), non-tumorigenic cells (e.g., normal cells), fetal cells, embryonic cells, adult cells, mitotic cells, non-mitotic cells, or any combination thereof.

In one aspect, provided herein is a pharmaceutical composition comprising: a circular RNA comprising, in the following order, a 3′ group I intron fragment, an Internal Ribosome Entry Site (IRES), an expression sequence encoding a polypeptide (e.g., a nucleobase editing system, therapeutic protein, such as a chimeric antigen receptor (CAR) or T cell receptor (TCR) complex protein), and a 5′ group I intron fragment, and a transfer vehicle comprising at least one of (i) an ionizable lipid, (ii) a structural lipid, and (iii) a PEG-modified lipid, wherein the transfer vehicle is capable of delivering the circular RNA polynucleotide to a cell (e.g., a human cell, such as an immune cell present in a human subject), such that the polypeptide is translated in the cell.

In some embodiments, the pharmaceutical composition is formulated for intravenous administration to the human subject in need thereof. In some embodiments, the 3′ group I intron fragment and 5′ group I intron fragment are Anabaena group I intron fragments.

In certain embodiments, the 3′ intron fragment and 5′ intron fragment are defined by the L9a-5 permutation site in the intact intron. In certain embodiments, the 3′ intron fragment and 5′ intron fragment are defined by the L8-2 permutation site in the intact intron.

In some embodiments, the IRES is from Taura syndrome virus, Tiiatoma virus, Theiler's encephalomyelitis virus, Simian Virus 40, Solenopsis invicta virus 1, Rhopalosiphum padi virus, Reticuloendotheliosis virus, Human poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picoma-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATiR, Human BAG-1, Human BCL2, Human BiP, Human c-IAPl, Human c-myc, Human eIF4G, Mouse NDST4L, Human LEF1, Mouse HIFI alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFTID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SHI, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA 16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistravirus, Hubei Picoma-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the IRES comprises a CVB3 IRES or a fragment or variant thereof. In some embodiments, the pharmaceutical composition comprises a first internal spacer between the 3′ group I intron fragment and the IRES, and a second internal spacer between the expression sequence and the 5′ group I intron fragment. In certain embodiments, the first and second internal spacers each have a length of about 10 to about 60 nucleotides.

In some embodiments, the circular mRNA comprises a nucleotide sequence encoding a polypeptide of interest, such as a nucleobase editing system or therapeutic protein (e.g., a CAR or TCR complex protein).

In embodiments where the therapeutic protein encoded by the herein RNA payload (e.g., circular or linear mRNA) is a CAR or TCR complex protein, the CAR or TCR complex protein comprises an antigen binding domain specific for an antigen selected from the group: CD 19, CD123, CD22, CD30, CD171, CS-1, C-type lectin-like molecule-1, CD33, epidermal growth factor receptor variant III (EGFRvIII), disialoganglioside GD2, disaloganglioside GD3, TNF receptor family member, B cell maturation antigen (BCMA), Tn antigen ((Tn Ag) or (GalNAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD 117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gplOO), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), claudin 18.2 (CLDN18.2), thyroid stimulating hormone receptor (TSHR), G protein-coupled receptor class C group 5, member D (GPRC5D), chromosome X open reading frame 61 (CXORF61), CD97, and CD179a.

In further embodiments where the therapeutic protein encoded by the herein RNA payload (e.g., circular or linear mRNA) is a CAR or TCR complex protein, the CAR or TCR complex protein comprises a CAR comprising an antigen binding domain specific for CD19. In some embodiments, the CAR or TCR complex protein comprises a CAR comprising a costimulatory domain selected from the group CD28, 4-1BB, 0X40, CD27, CD30, ICOS, GITR, CD40, CD2, SLAM, and combinations thereof. In some embodiments, the CAR or TCR complex protein comprises a CAR comprising a CD3zeta signaling domain. In some embodiments, the CAR or TCR complex protein comprises a CAR comprising a CH2CH3, CD28, and/or CD8 spacer domain. In some embodiments, the CAR or TCR complex protein comprises a CAR comprising a CD28 or CD8 transmembrane domain.

In some embodiments, the CAR or TCR complex protein comprises a CAR comprising: an antigen binding domain, a spacer domain, a transmembrane domain, a costimulatory domain, and an intracellular T cell signaling domain.

In some embodiments, the CAR or TCR complex protein comprises a multispecific CAR comprising antigen binding domains for at least two different antigens. In some embodiments, the CAR or TCR complex protein comprises a TCR complex protein selected from the group TCRalpha, TCRbeta, TCRgamma, and TCRdelta.

In some embodiments, the LNP-based nucleobase editing systems, RNA therapeutics and pharmaceutical compositions described herein further comprise a targeting moiety. In certain embodiments, the targeting moiety mediates receptor-mediated endocytosis or direct fusion of the delivery vehicle (LNPs) into selected cells of a selected cell population or tissue in the absence of cell isolation or purification. In certain embodiments, the targeting moiety is capable of binding to a protein selected from the group CD3, CD4, CD8, CDS, CD7, PD-1, 4-1BB, CD28, Clq, and CD2. In certain embodiments, the targeting moiety comprises an antibody specific for a macrophage, dendritic cell, NK cell, NKT, or T cell antigen. In certain embodiments, the targeting moiety comprises a scFv, nanobody, peptide, minibody, polynucleotide aptamer, heavy chain variable region, light chain variable region or fragment thereof.

In some embodiments, the LNP-based nucleobase editing systems, RNA therapeutics and pharmaceutical compositions described herein are administered in an amount effective to treat a disease in the human subject (e.g., wherein the disease can be cancer, muscle disorder, or CNS disorder, etc.). In some embodiments, the LNP-based nucleobase editing systems, RNA therapeutics and pharmaceutical compositions have an enhanced safety profile when compared to a pharmaceutical composition comprising T cells or vectors comprising exogenous DNA encoding the same polypeptide, e.g., a CAR complex protein.

In some embodiments, the LNP-based nucleobase editing systems and pharmaceutical compositions thereof are administered in an amount effective to mount an immunogenic response in a human subject for the vaccination against an infectious agent and/or cancer. In some embodiments, the LNP-based nucleobase editing systems and pharmaceutical compositions have an enhanced safety profile when compared to state of the art gene editing delivery compositions.

In another aspect, the present disclosure provides a circular RNA comprising, in the following order, a 3′ group I intron fragment, an Internal Ribosome Entry Site (IRES), an expression sequence encoding a polypeptide (e.g., a nucleobase editing system, therapeutic protein, such as a chimeric antigen receptor (CAR) or T cell receptor (TCR) complex protein), and a 5′ group I intron fragment.

In some embodiments, the 3′ group I intron fragment and 5′ group I intron fragment are Anabaena group I intron fragments. In certain embodiments, the 3′ intron fragment and 5′ intron fragment are defined by the L⁹a-5 permutation site in the intact intron.

In certain embodiments, the 3′ intron fragment and 5′ intron fragment are defined by the L⁸-2 permutation site in the intact intron. In certain embodiments, the IRES comprises a CVB3 IRES or a fragment or variant thereof.

In some embodiments, the circular RNA comprises a first internal spacer between the 3′ group I intron fragment and the IRES, and a second internal spacer between the expression sequence and the 5′ group I intron fragment.

In certain embodiments, the first and second internal spacers each have a length of about 10 to about 60 nucleotides.

In some embodiments, the circular RNA payload of the LNP-based nucleobase editing systems, RNA therapeutics and pharmaceutical compositions described herein consists of natural nucleotides. In some embodiments, the circular RNA further comprises a second expression sequence encoding a therapeutic protein. In some embodiments, the therapeutic protein comprises a checkpoint inhibitor. In certain embodiments, the therapeutic protein comprises a cytokine.

In some embodiments, the circular RNA payload of the LNP-based nucleobase editing systems, RNA therapeutics and pharmaceutical compositions described herein consists of natural nucleotides.

In some embodiments, the circular RNA payload LNP-based nucleobase editing systems, RNA therapeutics and pharmaceutical compositions described herein comprises a nucleotide sequence that is codon optimized, either partially or fully. In some embodiments, the circular RNA is optimized to lack at least one microRNA binding site present in an equivalent pre-optimized polynucleotide. In some embodiments, the circular RNA is optimized to lack at least one endonuclease susceptible site present in an equivalent pre-optimized polynucleotide. In some embodiments, the circular RNA is optimized to lack at least one RNA-editing susceptible site present in an equivalent pre-optimized polynucleotide.

In some embodiments, the circular RNA payload of the LNP-based nucleobase editing systems, RNA therapeutics and pharmaceutical compositions described herein has an in vivo functional half-life in humans greater than that of an equivalent linear RNA having the same expression sequence. In some embodiments, the circular RNA has a length of about 100 nucleotides to about 10 kilobases. In some embodiments, the circular RNA has a functional half-life of at least about 20 hours. In some embodiments, the circular RNA has a duration of therapeutic effect in a human cell of at least about 20 hours. In some embodiments, the circular RNA has a duration of therapeutic effect in a human cell greater than or equal to that of an equivalent linear RNA comprising the same expression sequence. In some embodiments, the circular RNA has a functional half-life in a human cell greater than or equal to that of an equivalent linear RNA comprising the same expression sequence.

In some embodiments, the circular RNA payload of the LNP-based nucleobase editing systems, RNA therapeutics and pharmaceutical compositions described herein has a half-life of at least that of a linear counterpart. In some embodiments, the oRNA has a half-life that is increased over that of a linear counterpart. In some embodiments, the half-life is increased by about 5%, 10%, 15%, 20%, 25%, 30%, 35%, 40%, 45%, 50%, or greater. In some embodiments, the oRNA has a half-life or persistence in a cell for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In some embodiments, the oRNA has a half-life or persistence in a cell for no more than about 10 mins to about 7 days, or no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 24 hours (1 day), 36 hours (1.5 days), 48 hours (2 days),60 hours (2.5 days), 72 hours (3 days), 4 days, 5 days, 6 days, or 7 days.

In some embodiments, the circular RNA payload of the LNP-based nucleobase editing systems, RNA therapeutics and pharmaceutical compositions described herein has a half-life or persistence in a cell while the cell is dividing. In some embodiments, the oRNA has a half-life or persistence in a cell post division.

In certain embodiments, the circular RNA payload of the LNP-based nucleobase editing systems, RNA therapeutics and pharmaceutical compositions described herein has a half-life or persistence in a dividing cell for greater than about 10 minutes to about 30 days, or at least about 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the circular RNA payload of the LNP-based nucleobase editing systems, RNA therapeutics and pharmaceutical compositions described herein modulates a cellular function, e.g., transiently or long term. In certain embodiments, the cellular function is stably altered, such as a modulation that persists for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer. In certain embodiments, the cellular function is transiently altered, e.g., such as a modulation that persists for no more than about 30 mins to about 7 days, or no more than about 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours (1 day), 36 hours (1.5 days), 48 hours (2 days), 60 hours (2.5 days), 72 hours(3 days), 4 days, 5 days, 6 days, or 7 days.

In some embodiments, the circular RNA payload of the LNP-based nucleobase editing systems, RNA therapeutics and pharmaceutical compositions described herein is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the oRNA may be of a sufficient size to accommodate a binding site for a ribosome.

In some embodiments, the maximum size of the circular RNA payload of the LNP-based nucleobase editing systems, RNA therapeutics and pharmaceutical compositions described herein may be limited by the ability of packaging and delivering the RNA to a target. In some embodiments, the size of the oRNA is a length sufficient to encode polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the circular RNA payload of the LNP-based nucleobase editing systems, RNA therapeutics and pharmaceutical compositions described herein comprises one or more elements described elsewhere herein. In some embodiments, the elements may be separated from one another by a spacer sequence or linker. In some embodiments, the elements may be separated from one another by 1 nucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides.

In some embodiments, one or more elements are contiguous with one another, e.g., lacking a spacer element.

In some embodiments, one or more elements is conformationally flexible. In some embodiments, the conformational flexibility is due to the sequence being substantially free of a secondary structure.

In some embodiments, the circular RNA payload of the LNP-based nucleobase editing systems, RNA therapeutics and pharmaceutical compositions described herein comprises a secondary or tertiary structure that accommodates a binding site for a ribosome, translation, or rolling circle translation.

In some embodiments, the circular RNA payload of the LNP-based nucleobase editing systems, RNA therapeutics and pharmaceutical compositions described herein comprises particular sequence characteristics. For example, the oRNA may comprise a particular nucleotide composition. In some such embodiments, the oRNA may include one or more purine rich regions (adenine or guanosine). In some such embodiments, the oRNA may include one or more purine rich regions (adenine or guanosine).In some embodiments, the oRNA may include one or more AU rich regions or elements (AREs). In some embodiments, the oRNA may include one or more adenine rich regions.

In some embodiments, the circular RNA payload of the LNP-based nucleobase editing systems, RNA therapeutics and pharmaceutical compositions described herein comprises one or more modifications described elsewhere herein.

In some embodiments, the circular RNA payload of the LNP-based nucleobase editing systems, RNA therapeutics and pharmaceutical compositions described herein comprises one or more expression sequences and is configured for persistent expression in a cell of a subject in vivo. In some embodiments, the oRNA is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Regulatory Elements

In some embodiments, the circular RNA payload of the LNP-based nucleobase editing systems, RNA therapeutics and pharmaceutical compositions described herein comprises one or more regulatory elements. As used herein, a “regulatory element” is a sequence that modifies expression of an expression sequence, e.g., a nucleotide sequence encoding a nucleobase editing system or a therapeutic protein, i.e., a coding region of interest (CROI). The regulatory element may include a sequence that is located adjacent to a coding region of interest encoded on the circular RNA payload. The regulatory element may be operatively linked to a nucleotide sequence of the circular RNA that encodes a coding region of interest (e.g., a nucleobase editing system or therapeutic polypeptide).

In some embodiments, a regulatory element may increase an amount of expression of a coding region of interest encoded on the circular RNA payload as compared to an amount expressed when no regulatory element exists.

In some embodiments, a regulatory element may comprise a sequence to selectively initiates or activates translation of a coding sequence of interest encoded on the circular RNA payload.

In some embodiments, a regulatory element may comprise a sequence to initiate degradation of the oRNA or the payload or cargo. Non-limiting examples of the sequence to initiate degradation includes, but is not limited to, riboswitch aptazyme and miRNA binding sites.

In some embodiments, a regulatory element can modulate translation of a coding region of interest encoded on the oRNA. The modulation can create an increase (enhancer) or decrease (suppressor) in the expression of the coding region of interest. The regulatory element may be located adjacent to the CROI (e.g., on one side or both sides of the CROI).

### Translation Initiation Sequence

In some embodiments, a translation initiation sequence functions as a regulatory element. In some embodiments, the translation initiation sequence comprises an AUG/ATG codon. In some embodiments, a translation initiation sequence comprises any eukaryotic start codon such as, but not limited to, AUG/ATG, CUG/CTG, GUG/GTG, UUG/TTG, ACG, AUC/ATC, AUU, AAG, AUA/ATA, or AGG. In some embodiments, a translation initiation sequence comprises a Kozak sequence. In some embodiments, translation begins at an alternative translation initiation sequence, e.g., translation initiation sequence other than AUG/ATG codon, under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translation initiation sequence, CUG/CTG. As another non-limiting example, the translation may begin at alternative translation initiation sequence, GUG/GTG. As yet another non-limiting example, the translation may begin at a repeat-associated non-AUG (RAN) sequence, such as an alternative translation initiation sequence that includes short stretches of repetitive RNA e.g. CGG, GGGGCC, CAG, CTG.

In some embodiments, the oRNA encodes a polypeptide or peptide and may comprise a translation initiation sequence. The translation initiation sequence may comprise, but is not limited to a start codon, a non-coding start codon, a Kozak sequence or a Shine-Dalgarno sequence. The translation initiation sequence may be located adjacent to the payload or cargo (e.g., on one side or both sides of the coding region of interest).

In some embodiments, the translation initiation sequence provides conformational flexibility to the oRNA. In some embodiments, the translation initiation sequence is within a substantially single stranded region of the oRNA.

The oRNA may include more than 1 start codon such as, but not limited to, at least 2, at least 3, at least 4, at least 5, at least 6, at least 7, at least 8, at least 9, at least 10, at least 11, at least 12, at least 13, at least 14, at least 15 or more than 15 start codons. Translation may initiate on the first start codon or may initiate downstream of the first start codon.

In some embodiments, the oRNA may initiate at a codon which is not the first start codon, e.g., AUG. Translation of the circular polyribonucleotide may initiate at an alternative translation initiation sequence, such as, but not limited to, ACG, AGG, AAG, CUG/CTG, GUG/GTG, AUA/ATA, AUU/ATT, UUG/TTG. In some embodiments, translation begins at an alternative translation initiation sequence under selective conditions, e.g., stress induced conditions. As a non-limiting example, the translation of the oRNA may begin at alternative translation initiation sequence, such as ACG. As another non-limiting example, the oRNA translation may begin at alternative translation initiation sequence, CUG/CTG. As yet another non-limiting example, the oRNA translation may begin at alternative translation initiation sequence, GTG/GUG. As yet another non-limiting example, the oRNA may begin translation at a repeat-associated non-AUG (RAN) sequence, such as an alternative translation initiation sequence that includes short stretches of repetitive RNA e.g. CGG, GGGGCC, CAG, CTG.

### IRES Sequences

In some embodiments, the oRNA described herein comprises an internal ribosome entry site (IRES) element capable of engaging an eukaryotic ribosome. In some embodiments, the IRES element is at least about 5 nucleotides, at least about 8 nucleotides, at least about 9 nucleotides, at least about 10 nucleotides, at least about 15 nucleotides, at least about 20 nucleotides, at least about 25 nucleotides, at least about 30 nucleotides, at least about 40 nucleotides, at least about 50 nucleotides, at least about 100 nucleotides, at least about 200 nucleotides, at least about 250 nucleotides, at least about 350 nucleotides, or at least about 500 nucleotides. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV_IGRpred, BQCV_IGRpred, BVDV1_1-385, BVDV1_29-391, CrPV_5NCR, CrPV_IGR, crTMV_IREScp, crTMV_IRESmp75, crTMV_IRESmp228, crTMV_IREScp, crTMV_IREScp, CSFV, CVB3, DCV_IGR, EMCV-R, EoPV_5NTR, ERAV 245-961, ERBV 162-920, EV711-748, FeLV-Notch2, FMDV_type_C, GBV-A, GBV-B, GBV-C, gypsy_env, gypsyD5, gypsyD2, HAV_HM175, HCV_type_1a, HiPV_IGRpred, HIV-1, HoCV1_IGRpred, HRV-2, IAPV_IGRpred, idefix, KBV_IGRpred, LINE-1_ORF1_-101_to_-1, LINE-1_ORF1-302_to_-202, LINE-I_ORF2-138_to_-86, LINE-I_ORF1_-44to_-1, PSIV_IGR, PV_type1_Mahoney, PV_type3_Leon, REV-A, RhPV_5NCR, RhPV_IGR, SINV1_IGRpred, SV40_661-830, TMEV, TMV_UI_IRESmp228, TRV_5NTR, TrV_IGR, or TSV_IGR. In some embodiments, the IRES element is at least partially derived from a cellular IRES, such as AML1/RUNX1, Antp-D, Antp-DE, Antp-CDE, Apaf-1, Apaf-1, AQP4, AT1R_var1, AT1R_var2, AT1R_var3, AT1R_var4, BAG1_p36delta236 nt, BAG1_p36, BCL2, BiP_-222_-3, c-IAP1_285-1399, c-IAP1_1313-1462, c-jun, c-myc, Cat-1224, CCND1, DAPS, eIF4G, eIF4GI-ext, eIF4GII, eIF4GII-long, ELG1, ELH, FGFIA,FMR1, Gtx-133-141, Gtx-1-166, Gtx-1-120, Gtx-1-196, hairless, HAP4, HIF1a, hSNM1, Hsp101, hsp70, hsp70, Hsp90, IGF2_leader2, Kv1.4_1.2, L-myc, LamB1_-335_-1, LEF1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kip1, 03_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP_5-464, XIAP_305-466, or YAP1.

In another embodiment, the IRES is an IRES sequence from Coxsackievirus B3 (CVB3), the protein coding region encodes Guassia luciferase (Gluc) and the spacer sequences are polyA-C.

In some embodiments, the IRES, if present, is at least about 50 nucleotides in length. In one embodiment, the vector comprises an IRES that comprises a natural sequence. In one embodiment, the vector comprises an IRES that comprises a synthetic sequence.

An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picornaviruses (e.g., FMDV), pest viruses (CFFV), polio viruses (PV), encephalomyocarditis viruses (ECMV), foot- and mouth disease viruses (FMDV), hepatitis C viruses (HCV), classical Swine fever viruses (CSFV), murine leukemia virus (MLV), simian immune deficiency viruses (SIV) or cricket paralysis viruses (CrPV).

### Termination Element

In some embodiments, the oRNA includes one or more coding regions of interest (i.e., also referred to as product expression sequences) which encode polypeptides of interest, including but not limited to nucleobase editing system and therapeutic proteins. In various embodiments, the product expression sequences may or may not have a termination element.

In some embodiments, the oRNA includes one or more product expression sequences that lack a termination element, such that the oRNA is continuously translated.

Exclusion of a termination element may result in rolling circle translation or continuous expression of the encoded peptides or polypeptides as the ribosome will not stall or fall-off. In such an embodiment, rolling circle translation expresses continuously through the product expression sequence.

In some embodiments, one or more product expression sequences in the oRNA comprise a termination element.

In some embodiments, not all of the product expression sequences in the oRNA comprise a termination element. In such instances, the product expression sequence may fall off the ribosome when the ribosome encounters the termination element and terminates translation.

### Rolling Circle Translation

In some embodiments, once translation of the oRNA is initiated, the ribosome bound to the oRNA does not disengage from the oRNA before finishing at least one round of translation of the oRNA. In some embodiments, the oRNA as described herein is competent for rolling circle translation. In some embodiments, during rolling circle translation, once translation of the oRNA is initiated, the ribosome bound to the oRNA does not disengage from the oRNA before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 10.sup.5 rounds, or at least 10.sup.6 rounds of translation of the oRNA.

In some embodiments, the rolling circle translation of the oRNA leads to generation of polypeptide that is translated from more than one round of translation of the oRNA. In some embodiments, the oRNA comprises a stagger element, and rolling circle translation of the oRNA leads to generation of polypeptide product that is generated from a single round of translation or less than a single round of translation of the oRNA.

### Circularization

In one embodiment, a linear RNA may be cyclized, or concatemerized. In some embodiments, the linear RNA may be cyclized in vitro prior to formulation and/or delivery. In some embodiments, the linear RNA may be cyclized within a cell.

In some embodiments, the mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′-NHS-ester moiety forming a new 5′-/3′-amide bond.

In the second route, T4 RNA ligase may be used to enzymatically link a 5′-phosphorylated nucleic acid molecule to the 3′-hydroxyl group of a nucleic acid forming a new phosphorodiester linkage. In an example reaction, \hat{ }g of a nucleic acid molecule is incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme may be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, the oRNA is made via circularization of a linear RNA.

In some embodiments, the following elements are operably connected to each other and, in some embodiments, arranged in the following sequence: a.) a 5′ homology arm, b.) a 3′ group I intron fragment containing a 3′ splice site dinucleotide, c.) a protein coding or noncoding region, d.) a 5′ group I intron fragment containing a 5′ splice site dinucleotide, and e.) a 3′ homology arm. In certain embodiments said vector allows production of a circular RNA that is translatable and/or biologically active inside eukaryotic cells. In some embodiments, the biologically active RNA is, for example, an miRNA sponge, or long noncoding RNA.

In some embodiments, the following elements are operably connected to each other and arranged in the following sequence: a.) a 5′ homology arm, b.) a 3′ group I intron fragment containing a 3′ splice site dinucleotide, c.) optionally, a 5′ spacer sequence, d.) optionally, an internal ribosome entry site (IRES), e.) a protein coding or noncoding region, f.) optionally, a 3′ spacer sequence, g.) a 5′ group I intron fragment containing a 5′ splice site dinucleotide, and h.) a 3′ homology arm. In certain embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) an internal ribosome entry site (IRES), e.) a protein coding or noncoding region, f.) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) a protein coding or noncoding region, e.) a 3′ spacer sequence, f.) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) an internal ribosome entry site (IRES), d.) a protein coding or noncoding region, e.) a 3′ spacer sequence, f) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a protein coding or noncoding region, d.) a 3′ spacer sequence, e.) a 5′ group I intron fragment containing a 5′ splice site dinucleotide, and f.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) a protein coding or noncoding region, e.) a 5′ group I intron fragment containing a 5′ splice site dinucleotide, and f.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) an internal ribosome entry site (IRES), d.) a protein coding or noncoding region, e.) a 5′ group I intron fragment containing a 5′ splice site dinucleotide, and f) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) an internal ribosome entry site (IRES), e.) a protein coding or noncoding region, f) a 3′ spacer sequence, g.) a 5′ group I intron fragment containing a 5′ splice site dinucleotide, and h.) a 3′ homology arm. In some embodiments, said vector allowing production of a circular RNA that is translatable and/or biologically active inside eukaryotic cells.

In one embodiment, the 3′ group I intron fragment and/or the 5′ group I intron fragment is from a Cyanobacterium Anabaena sp. pre-tRNA-Leu gene or T4 phage Td gene.

In one embodiment, the 3′ group I intron fragment and/or the 5′ group I intron fragment is from a Cyanobacterium Anabaena sp. pre-tRNA-Leu gene.

In one embodiment, the protein coding region encodes a protein of eukaryotic or prokaryotic origin. In another embodiment, the protein coding region encodes human protein or non-human protein. In some embodiments, the protein coding region encodes one or more antibodies. For example, in some embodiments, the protein coding region encodes human antibodies. In one embodiment, the protein coding region encodes a protein selected from hFIX, SP-B, VEGF-A, human methylmalonyl-CoA mutase (hMUT), CFTR, cancer self-antigens, and additional gene editing enzymes like Cpf1, zinc finger nucleases (ZFNs) and transcription activator-like effector nucleases (TALENs). In another embodiment, the protein coding region encodes a protein for therapeutic use. In one embodiment, the human antibody encoded by the protein coding region is an anti-HIV antibody. In one embodiment, the antibody encoded by the protein coding region is a bispecific antibody. In one embodiment, the bispecific antibody is specific for CD19 and CD22. In another embodiment, the bispecific antibody is specific for CD3 and CLDN6. In one embodiment, the protein coding region encodes a protein for diagnostic use. In one embodiment, the protein coding region encodes Gaussia luciferase (Gluc), Firefly luciferase (Fluc), enhanced green fluorescent protein (eGFP), human erythropoietin (hEPO), or Cas9 endonuclease.

In one embodiment, the 5′ homology arm is about 5-50 nucleotides in length. In another embodiment, the 5′ homology arm is about 9-19 nucleotides in length. In some embodiments, the 5′ homology arm is at least 5, 6, 7, 8, 9, 10, 11, 12, 13, 14, 15, 16, 17, 18 or 19 nucleotides in length. In some embodiments, the 5′ homology arm is no more than 50, 45, 40, 35, 30, 25 or 20 nucleotides in length. In some embodiments, the 5′ homology arm is 5, 6, 7, 8, 9, 10, 11, 12, 13, 14, 15, 16, 17, 18 or 19 nucleotides in length.

In one embodiment, the 3′ homology arm is about 5-50 nucleotides in length. In another embodiment, the 3′ homology arm is about 9-19 nucleotides in length. In some embodiments, the 3′ homology arm is at least 5, 6, 7, 8, 9, 10, 11, 12, 13, 14, 15, 16, 17, 18 or 19 nucleotides in length. In some embodiments, the 3′ homology arm is no more than 50, 45, 40, 35, 30, 25 or 20 nucleotides in length. In some embodiments, the 3′ homology arm is 5, 6, 7, 8, 9, 10, 11, 12, 13, 14, 15, 16, 17, 18 or 19 nucleotides in length.

In one embodiment, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19,20,21,22,23,24,25,26,27,28,29, 30, 31, 32, 33, 34, 35, 36, 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence.

In one embodiment, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is between 20 and 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 5′ spacer sequence is a polyA-C sequence.

### Extracellular Circularization

In some embodiments, the linear RNA is cyclized, or concatemerized using a chemical method to form an oRNA. In some chemical methods, the 5′-end and the 3′-end of the nucleic acid (e.g., a linear RNA) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear RNA is incubated at 37 C for 1 hour with 1-10 units of T4 RNA ligase according to the manufacturer's protocol. The ligation reaction may occur in the presence of a linear nucleic acid capable of base-pairing with both the 5′- and 3′-region in juxtaposition to assist the enzymatic ligation reaction. In one embodiment, the ligation is splint ligation where a single stranded polynucleotide (splint), like a single stranded RNA, can be designed to hybridize with both termini of a linear RNA, so that the two termini can be juxtaposed upon hybridization with the single-stranded splint. Splint ligase can thus catalyze the ligation of the juxtaposed two termini of the linear RNA, generating an oRNA.

In one embodiment, a DNA or RNA ligase may be used in the synthesis of the oRNA. As a non-limiting example, the ligase may be a circ ligase or circular ligase.

In one embodiment, either the 5′- or 3′-end of the linear RNA can encode a ligase ribozyme sequence such that during in vitro transcription, the resultant linear RNA includes an active ribozyme sequence capable of ligating the 5′-end of the linear RNA to the 3′-end of the linear RNA. The ligase ribozyme may be derived from the Group I Intron, Hepatitis Delta Virus, Hairpin ribozyme or may be selected by SELEX (systematic evolution of ligands by exponential enrichment).

In one embodiment, a linear RNA may be cyclized or concatemerized by using at least one non-nucleic acid moiety. In one aspect, the at least one non-nucleic acid moiety may react with regions or features near the 5′ terminus and/or near the 3′ terminus of the linear RNA in order to cyclize or concatermerize the linear RNA. In another aspect, the at least one non-nucleic acid moiety may be located in or linked to or near the 5′ terminus and/or the 3′ terminus of the linear RNA. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear RNA may be cyclized or concatemerized due to a non-nucleic acid moiety that causes an attraction between atoms, molecular surfaces at, near or linked to the 5′ and 3′ ends of the linear RNA. As a non-limiting example, one or more linear RNA may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In one embodiment, the linear RNA may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear RNA to cyclize or concatemerize. In another aspect, the peptides covalently linked to the ribozyme RNA near the 5′ terminus and the 3′ terminus may cause the linear RNA to cyclize or concatemerize after being subjected to ligated using various methods known in the art such as, but not limited to, protein ligation.

In some embodiments, the linear RNA may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear RNA into a 5′ monophosphate may occur by a two-step reaction comprising: (a) contacting the 5′ nucleotide of the linear RNA with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, RNA may be circularized using the methods described in WO2017222911 and WO2016197121, the contents of each of which are herein incorporated by reference in their entirety.

In some embodiments, RNA may be circularized, for example, by back splicing of a non-mammalian exogenous intron or splint ligation of the 5′ and 3′ends of a linear RNA. In one embodiment, the circular RNA is produced from a recombinant nucleic acid encoding the target RNA to be made circular. As a non-limiting example, the method comprises: a) producing a recombinant nucleic acid encoding the target RNA to be made circular, wherein the recombinant nucleic acid comprises in 5′ to 3′order: i) a 3′portion of an exogenous intron comprising a 3′ splice site, ii) a nucleic acid sequence encoding the target RNA, and iii) a 5′portion of an exogenous intron comprising a 5′splice site; b) performing transcription, whereby RNA is produced from the recombinant nucleic acid; and c) performing splicing of the RNA, whereby the RNA circularizes to produce a oRNA.

While not wishing to be bound by theory, circular RNAs generated with exogenous introns are recognized by the immune system as “non-self” and trigger an innate immune response. On the other hand, circular RNAs generated with endogenous introns are recognized by the immune system as “self” and generally do not provoke an innate immune response, even if carrying an exon comprising foreign RNA.

Accordingly, circular RNAs can be generated with either an endogenous or exogenous intron to control immunological self/non-self discrimination as desired. Numerous intron sequences from a wide variety of organisms and viruses are known and include sequences derived from genes encoding proteins, ribosomal RNA (rRNA), or transfer RNA (tRNA).

Circular RNAs can be produced from linear RNAs in a number of ways. In some embodiments, circular RNAs are produced from a linear RNA by backsplicing of a downstream 5′ splice site (splice donor) to an upstream 3′ splice site (splice acceptor). Circular RNAs can be generated in this manner by any nonmammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

In some embodiments, circular RNAs can be produced in vitro from a linear RNA by chemical or enzymatic ligation of the 5′ and 3′ ends of the RNA. Chemical ligation can be performed, for example, using cyanogen bromide (BrCN) or ethyl-3-(3′-dimethylaminopropyl) carbodiimide (EDC) for activation of a nucleotide phosphomonoester group to allow phosphodiester bond formation. See e.g., Sokolova (1988) FEBS Lett 232: 153-155; Dolinnaya et al. (1991) Nucleic Acids Res., 19:3067-3072; Fedorova (1996) Nucleosides Nucleotides Nucleic Acids 15: 1 137-1 147; herein incorporated by reference. Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl 2).

In some embodiments, splint ligation using an oligonucleotide splint that hybridizes with the two ends of a linear RNA can be used to bring the ends of the linear RNA together for ligation. Hybridization of the splint, which can be either a DNA or a RNA, orientates the 5′-phosphate and 3′-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, in some cases is more efficient than enzymatic ligation if the structure of the hybridized splint-RNA complex interferes with enzymatic activity.

In some embodiments, the oRNA may further comprise an internal ribosome entry site (IRES) operably linked to an RNA sequence encoding a polypeptide. Inclusion of an IRES permits the translation of one or more open reading frames from a circular RNA. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20: 102-110; Kobayashi et al., BioTechniques (1996) 21:399-402; and Mosser et al., BioTechniques 1997 22 150-161).

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

### Splicing Element

In some embodiments, the oRNA includes at least one splicing element. The splicing element can be a complete splicing element that can mediate splicing of the oRNA or the spicing element can be a residual splicing element from a completed splicing event. For instance, in some cases, a splicing element of a linear RNA can mediate a splicing event that results in circularization of the linear RNA, thereby the resultant oRNA comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the oRNA includes a splicing element adjacent each expression sequence. In some embodiments, the splicing element is on one or both sides of each expression sequence, leading to separation of the expression products, e.g., peptide(s) and or polypeptide(s).

In some embodiments, theoRNA includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the oRNA includes at least one repetitive nucleotide sequence described elsewhere herein as an internal splicing element. In such embodiments, the repetitive nucleotide sequence may include repeated sequences from the Alu family of introns. See, e.g., U.S. Pat. No. 11,058,706.

In some embodiments, the oRNA may include canonical splice sites that flank head-to-tail junctions of the oRNA.

In some embodiments, the oRNA may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′-OH group onto the 2′, 3′-cyclic phosphate of the same molecule forming a 3′, 5′-phosphodiester bridge.

In some embodiments, the oRNA may include a sequence that mediates self-ligation. Non-limiting examples of sequences that can mediate self-ligation include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear RNA may include complementary sequences, including either repetitive or nonrepetitive nucleic acid sequences within individual introns or across flanking introns. In some embodiments, the oRNA includes a repetitive nucleic acid sequence. In some embodiments, the repetitive nucleotide sequence includes poly CA or poly UG sequences. In some embodiments, the oRNA includes at least one repetitive nucleic acid sequence that hybridizes to a complementary repetitive nucleic acid sequence in another segment of the oRNA, with the hybridized segment forming an internal double strand. In some embodiments, repetitive nucleic acid sequences and complementary repetitive nucleic acid sequences from two separate oRNA that hybridize to generate a single oRNA, with the hybridized segments forming internal double strands. In some embodiments, the complementary sequences are found at the 5′ and 3′ ends of the linear RNA. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the oRNA. Such methods may include, but are not limited to click chemistry (e.g., alkyne- and azide-based methods, or clickable bases), olefin metathesis, phosphoramidate ligation, hemiaminal-imine crosslinking, base modification, and any combination thereof. In some embodiments, enzymatic methods of circularization may be used to generate the oRNA. In some embodiments, a ligation enzyme, e.g., DNA or RNA ligase, may be used to generate a template of the oRNA or complement, a complementary strand of the oRNA, or the oRNA. Any of the circular polynucleotides as taught in for example U.S. Pat. No. 10,709,779, which is incorporated by reference herein in its entirety, may be used herein. In addition, any of the circular RNAs, methods for making circular RNAs, circular RNA compositions that are described in the following publications are contemplated herein and are incorporated by reference in their entireties are part of the instant specification: U.S. Pat. Nos. 11,352,640, 11,352,641, 11,203,767, 10,683,498, 5,773,244, and 5,766,903; US Application Publications US 2022/0177540, US 2021/0371494, US 2022/0090137, US 2019/0345503, and US 2015/0299702; and PCT Application Publications WO 2021/226597, WO 2019/236673, WO 2017/222911, WO2016/187583, WO2014/082644 and WO 1997/007825.

### D. Gene Editing Systems

As described herein in various embodiments, the LNPs of the present disclosure may comprise a gene editing system. As used herein, the term “gene editing system” generally refers to a composition having one or more gene editing system components which are capable of independently or co-dependently editing, modifying, or altering a target polynucleotide sequence or a target transcript comprising a nucleic acid sequence and/or modifying the epigenome to effect a change in gene regulation. Gene editing systems for the present disclosure include any editing systems known in the art.

For example, the LNP compositions herein may be used to deliver any gene editing system including CRISPR (clustered regularly interspaced short palindromic repeats) and the associated CRISPR-associated proteins (e.g., CRISPR-Cas9) (Jinek et al., “A programmable dual-RNA-guided DNA endonuclease in adaptive bacterial immunity,” Science, Vol. 337 (6096), pp. 816-821), meganuclease editors (Boissel et al., “megaTALs: a rare-cleaving nuclease architecture for therapeutic genome engineering,” Nucleic Acids Research 42: pp. 2591-2601) and bacterial retron systems (Schubert et al., “High-throughput functional variant screens via in vivo production of single-stranded DNA,” PNAS, Apr. 27, 2021, Vol. 118(18), pp. 1-10). In particular, CRISPR-Cas9 has been derivatized in numerous ways to expand upon its guide RNA-based programmable double-strand cutting activity to form systems ranging from finding alternative CRISPR Cas nuclease enzymes having different PAM requirements and cutting properties (e.g., Cas12a, Cas12f, Cas13a, and Cas13b) to base editing (Komor et al, “Programmable editing of a target base in genomic DNA without double-stranded DNA cleavage,” Nature, May 19, 2016, 533 (7603); pp. 420-424 [cytosine base editors or CBEs] and Gaudelli et al., “Programmable base editing of A-T to G-C in genomic DNA without DNA cleavage,” Nature, Vol. 551, pp. 464-471 [adenine base editors or ABEs]) to prime editing (Anzalone et al., “Search-and-replace genome editing without double-strand breaks or donor DNA,” Nature, December 2019, 576 (7789): pp. 149-157) to twin prime editing (Anzalone et al., “Programmable deletion, replacement, integration and inversion of large DNA sequences with twin prime editing,” Nature Biotechnology, Dec. 9, 2021, vol. 40, pp. 731-740) to epigenetic editing (Kungulovski and Jeltsch, “Epigenome Editing: State of the Art, Concepts, and Perspective,” Trends in Genetics, Vol. 32, 206. pp. 101-113) to CRISPR-directed integrase editing (Yarnell et al., “Drag-and-drop genome insertion of large sequences without double-stranded DNA cleavage using CRISPR-directed integrases,” Nature Biotechnology, Nov. 24, 2022, (“PASTE”). Each of these gene editing systems may be packaged up in the LNP compositions described herein and delivered to target organs, tissues, and cells to bring about the modification of a target sequence or the expression of a target gene.

The gene editing systems deliverable by the herein disclosed LNPs can be any gene editing system. The gene editing systems contemplated herein can include (A) nucleobase gene editing systems which result in one or more the modifications to the sequence of target nucleic acid molecule, (B) an epigenetic editing system which results in one or more modifications to the epigenome to bring about an effect on gene expression without altering the sequence of a nucleic acid molecule, and (C) gene editing systems that combine the features of nucleobase editing systems and epigenetic editing systems.

Nucleobase editing systems include a wide array of configurations with various combinations of protein functionalities and/or nucleic acid molecule components, all of which are contemplated herein. In general, nucleobase editing systems comprise at least a (i) DNA binding domain that is user-programmable to target a specific sequence in a nucleic acid molecule and optionally (ii) one or more effector domains that facilitate the modification of the sequence of the nucleic acid molecule. User-programmability may comprise amino acid sequence-programmable DNA binding domains (e.g., TALENS and zinc finger-binding domains) or nucleic acid sequence-programmable DNA binding domains (e.g., CRISPR Cas9, CRISPR Cas12a, CRISPR Cas12f, CRISPR Cas13a, CRISPR Cas13b, IscB, IsrB, or TnpB). Similarly, epigenetic editing systems comprise at least a (i) DNA binding domain that targets a specific sequence in a nucleic acid molecule and (ii) one or more effector domains that facilitates the modification of one or more epigenomic features of the nucleic acid molecule.

Gene editing systems may also comprise one or more effector domains that provide various functionalities that facilitate changes in nucleotide sequence and/or gene expression, such as, but not limited to, single-strand DNA binding proteins, nucleases, endonucleases, exonucleases, deaminases (e.g., cytidine deaminases or adenosine deaminases), polymerases (e.g., reverse transcriptases), integrases, recombinases, etc., and fusion proteins comprising one or more functional domains linked together). In certain embodiments, the nucleobase editing systems include, but are not limited to, systems comprising a clustered regularly interspaced short palindromic repeats (“CRISPR”)-associated (“Cas”) protein, a zinc finger nuclease (“ZFN”), a transcription activator-like effector nuclease (“TALEN”), an adenosine deaminase acting on RNA (“ADAR”) enzyme, an adenosine deaminase acting on transfer RNA (“ADAT”) enzyme, an activation induced cytidine deaminase (“AID”)/apolipoprotein B editing complex (“APOBEC”) enzyme, a meganuclease, IscB, IsrB, TnpB, or a restriction enzyme.

In some embodiments, the nucleobase editing system edits, modifies, or alters the target polynucleotide sequence ex vivo. In some embodiments, the nucleobase editing system edits, modifies, or alters the target polynucleotide sequence in vivo. In some embodiments, the nucleobase editing system edits, modifies, or alters the target polynucleotide sequence in a cell. In some embodiments, the cell is a mammalian cell. In some embodiments, the cell is a human cell.

In some embodiments, the target polynucleotide sequence is a gene or a regulatory sequence that controls transcription of a gene (e.g., a promoter, transcription binding site, enhancer sequence, etc.) or a sequence which controls the translation of a messenger RNA. In some embodiments, the target transcript comprising a nucleic acid sequence is a product of gene transcription. In some embodiments, the target transcript comprising a nucleic acid sequence is an RNA transcript such as a messenger RNA transcript, microRNA transcript or transfer RNA transcript.

The originator constructs and benchmark constructs of the present disclosure may comprise, encode or be conjugated to a cargo which is a nucleobase editing tool. As used herein, the term “nucleobase editing tool” is used interchangeably with “nucleobase editing system component” and generally refers to a compound or substance which is capable of independently or co-dependently editing, modifying, or altering a target polynucleotide sequence or a target transcript comprising a nucleic acid sequence. Nucleobase editing tools for the present disclosure include all nucleobase editing tools known in the art. In certain embodiments, the nucleobase editing tools include, but not limited to, effector proteins which modify DNA or RNA, guide elements which guide effector proteins to specific DNA or RNA sequence, repair elements which encode a nucleic acid sequence template, and supportive elements which activate or modulate the activity of another nucleobase editing tool, or activates or modulates host DNA repair enzymes.

In some embodiments, the cargo may comprise a nucleobase editing tool or a polynucleotide encoding a nucleobase editing tool. In some embodiments, the cargo may comprise one or more polynucleotides encoding a nucleobase editing tool. In some embodiments, the cargo may comprise a polynucleotide encoding one or more nucleobase editing tools. In some embodiments, the cargo may comprise a polynucleotide that is a component of the nucleobase editing tool. In some embodiments, the cargo may comprise a polynucleotide encoding one or more protein or peptide components in the nucleobase editing tool.

In some embodiments, the cargo may comprise an effector protein capable of modifying a target DNA or RNA sequence. In some embodiments, the cargo may comprise a polynucleotide encoding an effector protein. In certain embodiments, the effector proteins include polymerases, nucleases, mutator enzymes, reverse transcriptases, recombinases, integrases, endonucleases, exonucleases, transposases, and deaminases. As used herein, the term “polymerases,” includes enzymes which catalyze the synthesis of DNA or RNA polymers. As used herein, the term “nucleases,” includes enzymes which cleave nucleobases. In certain embodiments, nucleases include enzymes which create single-stranded breaks (“SSB”) or double-stranded breaks (“DSB”) in nucleic acid sequences. As used herein, the term “mutator enzymes,” in its broadest sense, includes enzymes which mutate nucleic acid sequences. In certain embodiments, the cargo may comprise nucleases such as effector proteins include clustered regularly interspaced short palindromic repeats (“CRISPR”)-associated (“Cas”) proteins, zinc finger nucleases (“ZFNs”), transcription activator-like effector nucleases (“TALENs”), adenosine deaninase acting on RNA (“ADAR”) enzymes, adenosine deaminase acting on transfer RNA (“ADAT”) enzymes, activation induced cytidine deaminase (“AID”)/apolipoprotein B editing complex (“APOBEC”) enzymes, meganucleases, IscB, IsrB, TnpB, or restriction enzymes.

In some embodiments, the cargo may comprise a guide element which guide effector proteins to target a DNA or RNA sequence. In some embodiments, the cargo may comprise a polynucleotide encoding a guide element. In certain embodiments, guide elements include guide RNAs (“gRNAs”), CRISPR RNAs (“crRNAs”), prime editing guide RNAs (“pegRNAs”), transcription activator-like effectors (TALEs), or antisense oligomers.

In some embodiments, the cargo may further comprise a repair element which encodes a sequence repair template. In some embodiments, the cargo may further comprise a polynucleotide encoding a repair element or sequence repair template.

In some embodiments, the cargo may further comprise a supportive element which activates or modulates the editing system. In some embodiments, the cargo may further comprise a supportive element which activates or modulates the effector protein. In some embodiments, the cargo may further comprise a polynucleotide encoding a supportive element. Non-limiting categories of supportive elements include trans-activating RNA (“tracrRNA”).

### CRISPR-Cas Editors

In some embodiments, the LNPs may be used to deliver a CRISPR-Cas gene editing system comprising a CRISPR-Cas programmable nuclease, such as a CRISPR-Cas9 or CRISPR-Cas12a nuclease.

In general, nucleobase editing systems comprise at least a (i) DNA binding domain that is user-programmable to target a specific sequence in a nucleic acid molecule and optionally (ii) one or more effector domains that facilitate the modification of the sequence of the nucleic acid molecule. User-programmability may comprise amino acid sequence-programmable DNA binding domains (e.g., TALENS and zinc finger-binding domains) or nucleic acid sequence-programmable DNA binding domains (e.g., CRISPR Cas9, CRISPR Cas12a, CRISPR Cas12f, CRISPR Cas13a, CRISPR Cas13b, or TnpB), and including a guide RNA which targets the programmable DNA binding protein to target sequence.

In some embodiments, the CRISPR-Cas system comprises a Cas or Cas-derived protein.

In other embodiments, the sequence-programmable DNA binding domains (e.g., RNA-guided nuclease) used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system Class 1, Type I, II, or III Cas nucleases; Class 2, Type II nuclease (such as Cas9); a Class 2, Type V nuclease (such as Cpf1), or a Class 2, Type VI nuclease (such as C2c2). Examples of Cas proteins include Casl, CaslB, Cas2, Cas3, Cas4, Cas5, Cas5e (CasD), Cas6, Cas6e, Cas6f, Cas7, Cas8al, Cas8a2, Cas8b, Cas8c, Cas9 (Csnl or Csxl2), CaslO, CaslOd, CasF, CasG, CasH, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csx14, CsxlO, Csx16, CsaX, Csx3, Csxl, Csxl5, Csfl, Csf2, Csf3, Csf4, and Cul966, and homologs or modified versions thereof. In some embodiments, an LNP of the present disclosure comprises gene editing system is or comprises a Type V nuclease editing system described in International Application Publication WO2024020346A1, which is incorporated by reference herein in its entirety.

In some embodiments, a Class 1, type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP 002989955, WP_038434062, WP_011528583); Campylobacter jejuni (WP 022552435, YP 002344900), Campylobacter coli (WP 060786116); Campylobacter fetus (WP 059434633); Corynebacterium ulcerans (NC_015683, NC_017317); Corynebacterium diphtheria (NC_016782, NC_016786); Enterococcus faecalis (WP 033919308); Spiroplasma syrphidicola (NC 021284); Prevotella intermedia (NC 017861); Spiroplasma taiwanense (NC 021846); Streptococcus iniae (NC 021314); Belliella baltica (NC 018010); Psychroflexus torquisl (NC 0 18721); Streptococcus thermophilus (YP 820832), Streptococcus mutans (WP 061046374, WP 024786433); Listeria innocua (NP 472073); Listeria monocytogenes (WP 061665472); Legionella pneumophila (WP 062726656); Staphylococcus aureus (WP_001573634); Francisella tularensis (WP_032729892, WP_014548420), Enterococcus faecalis (WP 033919308); Lactobacillus rhamnosus (WP 048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacterid. 198(5): 797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

In another embodiment, the gene editing system delivered by the LNP-based RNA medicines described herein may comprise a CRISPR-Cas12a (Cpf1) nuclease. Cpf1 was first identified from Prevotella and Francisella 1 (Cpf1, or Cas12a) and published in Zetsche et al., “Cpf1 is a single RNA-guided endonuclease of a class 2 CRISPR-Cas system,” Cell, Oct. 22, 2015, 163, pp. 759-711, which is incorporated herein by reference. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

The gene editing systems described herein can include any known Cas12a nuclease, or any variant thereof, such as any Cas12a ortholog described in U.S. patent application Ser. No. 18/297,346, U.S. patent application Ser. No. 18/481,393, or International Application Publication WO2024020346A1, or an amino acid sequence having at least 75%, 80%, 85%, 90%, 95%, 99%, or up to 100% sequence identity with any of the Cas12a orthologs described in said patent applications.

The gene editing systems described herein can include any known Cas12a nuclease, or any variant thereof, such as any Cas12a ortholog described in (1) Wu J, Gao P, Shi Y, Zhang C, Tong X, Fan H, Zhou X. Zhang Y, Yin H1. Characterization of a thermostable. Cas12a ortholog. Cell Insight 2023 October 11; 2(6):100126. doi: 10.1016/j.cellin.2023.100126. PMID: 38047138; PMCID: PMC10692460; (2) Swarts DC, Jinek M. Cas9 versus Cas12a/Cpf1: Structure-function comparisons and implications for genome editing. Wiley Interdiscip Rev RNA. 2018 September; 9(5):e148L. doi: 10.J002/wrna.1481. Epub 2018 May 22. PMID: 29790280; (3) Kleinstiver B P, Sousa A A, Walton R T, Tak Y E, Hsu J Y, Clement K, Welch M M, Horng J E, Malagon-Lopez J, Scarto I, Maus M V, Pinello L, Aryee M J Joung J K. Engineered CRISPR-Cas12a variants with increased activities and improved targeting ranges for gene, epigenetic and base editing. Nat Biotechnol 2019 March; 37(3):276-282. doi: 10.1038/s41587-018-0011-0. Epub 2019 Feb. 11. Erratum in: Nat Biotechnol 2020 July; 38(7):901. PMID: 30742127; PMCID: PMC6401248; (4) Ma E. Chen K, Shi II, Stahl E C, Adler B, Trinidad M, Liu J, Zhou K, Ye J, Doudna J A. Improved genome editing by an engineered CRISPR-Cas12a. Nucleic Acids Res. 2022 Dec. 9; 50(22):12689-12701. doi: 10.1093/nar/gkac1192. PMID: 36537251; PMC1D: PMC9825149; (5) Li T, Zhu L, Xiao B, Gong Z, Liao Q, Guo J. CRISPR-Cpf1-mediated genome editing and gene regulation in human cells. Biotechnol Adv. 2019 January-February; 37(1):21-27. doi: 10.1016/j.biotechadv.2018.10.013. Epub 2018 Nov. 3. PMID: 30399413 (6); Jacobsen T, Liao C, Beisel C L. The Acidaminococcus sp. Cas12a nuclease recognizes GTTV and GCTV as non-canonical PAMs. FEMS Microbiol Lett. 2019 Apr. 1; 366(8):fnz085. doi: 10.1093/femsle/fnz085. PMID: 31004485; PMCID: PMC6604746; (7) Huang 11, Huang G, Tan Z, Hu Y, Shan L, Zhou J, Zhang X, Ma S, Lv W, Huang T. Liu Y, Wang D. Zhao X, Lin Y, Rong Z. Engineered Cas12a-Plus nuclease enables gene editing with enhanced activity and specificity. BMC Biol. 2022 Apr. 25; 20(1):91. doi: 10.1 186/s12915-022-01296-1. PMID: 35468792; PMCID: PMC9040236; (8) Zhu D, Wang J, Yang D, Xi J, Li J. High-Throughput Profiling of Cas12a Orthologues and Engineered Variants for Enhanced Genome Editing Activity. Int J Mol Sci. 2021 Dec. 10; 22(24):13301. doi: 10.3390/ijms222413301. PMID: 34948095; PMCID: PMC8706968; (9) Gao L, Cox DET, Yan W X, Manteiga J C, Schneider M W, Yamano T, Nishimasu H, Nureki O, Crosetto N, Zhang F. Engineered Cpf variants with altered PAM specificities. Nat Biotechnol. 2017 August; 35(8):789-792. doi: 10.1038/nbt.3900. Epub 2017 Jun. 5. PMID: 28581492; PMCID: PMC5548640; (10) Zhang L, Zuris J A, Viswanathan R, Edelstein J N, Turk R, Thommandru B, Rube H T, Glenn S E, Collingwood M A, Bode N M, Beaudoin S F, Lele S, Scott S N, Wasko K M, Sexton S, Borges C M, Schubert M S, Kurgan G L, McNeill M S, Fernandez C A, Myer V E, Morgan R A, Behlke M A, Vakulskas C A, AsCas12a ultra nuclease Facilitates the rapid generation of therapeutic cell medicines. Nat Commun. 2021 Jun. 23; 12c1):3908. doi: 10.1038/s41467-021-24017-8. Erratum in: Nat Commun. 2021 Jul. 19; 12(1):4500. PMID: 34162850; PMCID: PMC8222333; (11) Ling X, Chang L, Chen H. Gao X, Yin J, Zuo Y, Huang Y. Zhang B, Hu J. Liu T. Improving the efficiency of CRISPR-Cas12a-based genome editing with site-specific covalent Cas12a-crRNA conjugates. Mol Cell. 2021 Nov. 18; 81(22):4747-4756.e7, doi: 10.1016/j.molccl2021.09.021. Epub 2021 Oct. 13. PMID: 34648747; and (12) Zhou J, Chen P, Wang H, Liu H, Li Y, Zhang Y, Wu Y, Pack C, Sun Z, Lei J. Yin L. Cas12a variants designed for lower genome-wide off-target effect through stringent PAM recognition. Mol Ther. 2022 Jan. 5; 30(i):244-255. doi: 10.101 6/j.ymthe.2021.10.010. Epub 2021 Oct. 20. PMID: 34687846; PMCID: PMC8753454; each of which are incorporated by reference in their entireties.

Any publicly known Cas12a/Cpf1 may be used as a component of the gene editing systems described herein, including but not limited to, the following publicly available amino acid sequences: GenBank Accession No. QOE76068.1, GenBank Accession No. WKU83685.1; GenBank Accession No. WBC51234.1; GenBank Accession No. QOL02411.1; GenBank Accession No. UVJ64960.1; GenBank Accession No. UVJ64952.1; GenBank Accession No. UVJ64951.1; GenBank Accession No. UVJ64956.1; GenBank Accession No. UVJ64954.1; GenBank Accession No. UVJ64953.1; GenBank Accession No. UVJ64952.1; GenBank Accession No. UVJ64951.1; GenBank Accession No. UVJ64949.1; GenBank Accession No. UVJ64947.1; GenBank Accession No. UVJ64946.1; GenBank Accession No. UVJ64945.1; GenBank Accession No. WP_320869194.1; GenBank Accession No. WOZ85592.1; GenBank Accession No. WOF96262.1; and any amino acid sequence having at least 75%, 80%, 85%, 90%, 95%, 99%, or up to 100% sequence identity with any of the aforementioned sequence, or any other publicly available Cas12a sequence.

C2c1 (Cas12b) is another class II CRISPR/Cas system RNA-guided nuclease that may be used. C2c1, similarly to Cas9, depends on both a crRNA and tracrRNA for guidance to target sites. See, e.g., Shmakov et al. (2015) Mol Cell. 60(3):385-397, Zhang et al. (2017) Front Plant Sci. 8:177; herein incorporated by reference.

In one aspect, a nucleic acid sequence-programmable DNA binding domain can be associated with or complexed with at least one guide nucleic acid (e.g., guide RNA or a pegRNA), which localizes the DNA binding domain to a DNA sequence that comprises a DNA strand (i.e., a target strand) that is complementary to the guide nucleic acid, or a portion thereof (e.g., the spacer of a guide RNA which anneals to the protospacer of the DNA target). In other words, the guide nucleic-acid “programs” the DNA binding domain (e.g., Cas9 or equivalent) to localize and bind to complementary sequence of the protospacer in the DNA.

Any suitable nucleic acid sequence-programmable DNA binding domain may be used in the prime editors described herein. In various embodiments, the nucleic acid sequence-programmable DNA binding domain may be any Class 2 CRISPR-Cas system, including any type II, type V, or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. CRISPR-Cas nomenclature is extensively discussed in Makarova et al., “Classification and Nomenclature of CRISPR-Cas Systems: Where from Here?,” The CRISPR Journal, Vol. 1. No. 5, 2018, the entire contents of which are incorporated herein by reference.

Without being bound by theory, the mechanism of action of certain CRISPR Cas enzymes contemplated herein includes the step of forming an R-loop whereby the Cas protein induces the unwinding of a double-strand DNA target, thereby separating the strands in the region bound by the Cas protein. The guide RNA spacer then hybridizes to the “target strand” at a region that is complementary to the protospacer sequence of the DNA. In some embodiments, the Cas protein may include one or more nuclease activities, which then cut the DNA leaving various types of lesions. For example, the Cas protein may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand.

Exemplary Cas proteins with different nuclease activities include “Cas9 nickase” (“nCas9”) and a deactivated Cas9 having no nuclease activities (“dead Cas9” or “dCas9”).

The below description of various Cas proteins which can be used in connection with the presently disclosed LNP-delivered gene editing systems is not meant to be limiting in any way. The gene editing systems may comprise the canonical SpCas9 or Cas12a, or any ortholog Cas9 protein or Cas12a protein, or any variant Cas9 or Cas12a protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave one strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure.

The gene editing systems described herein may also comprise Cas9 equivalents, including Cas12a (Cpf1) and Cas12bl proteins. The Cas proteins usable herein (e.g., SpCas9, Cas9 variant, or Cas9 equivalents) may also contain various modifications that alter/enhance their PAM specificities. The present disclosure contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence of Streptococcus pyogenes M1 (Accession No. Q99ZW2).

The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any Class 2 CRISPR system (e.g., type II, V, VI), including Cas12a (Cpf1), Cas12e (CasX), Cas12bl (C2c1), Cas12b2, Cas12c (C2c3), C2c4, C2c8, C2c5, C2c10, C2c9 Cas13a (C2c2), Cas13d, Cas13c (C2c7), Cas13b (C2c6), and Cas13b.

Further Cas-equivalents are described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described in the art and are incorporated herein by reference. As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

In certain embodiments, a polynucleotide programmable nucleotide binding domain of a nucleobase editor itself comprises one or more domains. In one embodiment, a polynucleotide programmable nucleotide binding domain comprises one or more nuclease domains. In some embodiments, the nuclease domain of a polynucleotide programmable nucleotide binding domain comprises an endonuclease or an exonuclease. In some embodiments, the endonuclease cleaves a single strand of a double-stranded nucleobase. In some embodiments, the endonuclease cleaves both strands of a double-stranded nucleobase molecule. In some embodiments, the polynucleotide programmable nucleotide binding domain is a deoxyribonuclease. In some embodiments, the polynucleotide programmable nucleotide binding domain is a ribonuclease.

In some embodiments, the nuclease domain of a polynucleotide programmable nucleotide binding domain can cut zero, one, or two strands of a target polynucleotide. In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleobase molecule (e.g., DNA). In some embodiments, the nickase is derived from a fully catalytically active (e.g., natural) form of a polynucleotide programmable nucleotide binding domain by introducing one or more mutations into the active polynucleotide programmable nucleotide binding domain. In certain embodiments, where a polynucleotide programmable nucleotide binding domain comprises a nickase domain derived from Cas9. Cas12a and Cas9 nickases are known in the art and contemplated for use herein, for example as discussed in (1) Schubert MS, Thommandru B, Woodley J, Turk R, Yan S. Kurgan G-, McNeill MS, Rettig GR. Optimized design parameters for CRISPR Cas9 and Cas12a homology-directed repair. Sci Rep, 2021 Sep. 30; 11(I):19482. doi: 10.1038/s41598-021-98965-y. PMID: 34593942; PMCID: PMC8484621; (2) Fu B X H, Smith J D, Fuchs R T, Mabuchi M, Curcuru J, Robb G B, Fire A Z. Target-dependent nickase activities of the CRISPR-Cas nucleases Cpf1 and Cas9. Nat Microbiol. 2019 May; 4(5):888-897. doi: 10.1038/s41564-019-0382-0. Epub 2019 Mar. 4. PMID: 30833733; PMCID: PMC6512873; (3) Xu T, Tao X, Kempher ML, Zhou J. Cas9 Nickase-Based Genome Editing in Clostridium cellulolyticum. Methods Mol Biol. 20222479:227-243. doi: 10.1007/978-1-0716-2233-9_15. PMID: 35583742; (4) Wu W H, Ma X M, Huang J Q, Lai Q, Jiang F N, Zou CY, Chen L T, Y u L. CRISPR/Cas9 (D10A) nickase-mediated Hb CS gene editing and genetically modified fibroblast identification. Bioengineered. 2022 May; 13(5):13398-13406. doi: 10.1080/21655979.2022.2069940. PMID: 36700476; PMCID: PMC9276056: each of which are incorporated herein by reference in their entireties.

In some embodiments, the Cas9-derived nickase has one or more mutations in the RuvC-1 domain. In one embodiment, the Cas9-derived nickase has a D10A mutation in the RuvC-1 domain. In some embodiments, the Cas9-derived nickase has one or more mutations in the REC Lobe domain. In one embodiment, the Cas9-derived nickase has a N497A, R661A, and/or Q695A mutation in the REC Lobe domain. In some embodiment, the Cas9-derived nickase has one or more mutations in the HNH domain. In one embodiment, the Cas9-derived nickase has H840A, N863A, and/or D839A in the IINII domain.

In certain embodiments, in the SpCas9-derived nickase, the residue H840 retains catalytic activity and can thereby cleave a single strand of the nucleobase duplex. In certain embodiments, a Cas9-derived nickase domain can comprise an H840A mutation, while the amino acid residue at position 10 remains a D. In certain embodiments, a Cas9-derived nickase domain can comprise an N863A mutation, while the amino acid residue at position 10 remains a D. In some embodiments, the nickase is derived from a fully catalytically active (e.g., natural) form of a polynucleotide programmable nucleotide binding domain by removing all or a portion of a nuclease domain that is not required for the nickase activity. In certain embodiments, where a polynucleotide programmable nucleotide binding domain comprises a nickase domain derived from Cas9, the Cas9-derived nickase domain comprises a deletion of all or a portion of the RuvC domain or the HNH domain.

Any of the above CRISPR-Cas editor embodiments or any variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### Base Editors

In other embodiments, the LNPs may be used to deliver a base editing system. Base editors are generally composed of an engineered deaminase and a catalytically impaired CRISPR-Cas9 variant and enzymatically convert one base to another base at a specific target site with the assistance of endogenous DNA repair systems in the cell. However, base editors may also comprise Cas12a enzymes and/or other programmable nucleases. For example, Cas12a-configured base editors are described in (1) Chen F, Lian M, Ma B. Gou S, Luo X, Yang K, Shi H, Xic J, Ge W, Ouyang Z, Lai C, Li N, Zhang Q, Jin Q, Liang Y, Chen T, Wang J, Zhao X, Li L, Yu M, Ye Y, Wang K, Wu II, Lai L. Multiplexed base editing through Cas12a variant-mediated cytosine and adenine base editors. Commun Biol. 2022 Nov. 2; 5(1):1 163. doi: 10.1038/42003-022-04152-8. PMID: 36323848; PMCID: PMC9630288; (2) Wang X, Ding C, Yu W, Wang Y, He S, Yang B, Xiong YC, Wei J, Li J, Liang J, Lu Z, Zhu W, Wu J, Zhou Z, Huang X, Liu Z, Yang L, Chen J, Cas12a Base Editors Induce Efficient and Specific Editing with Low DNA Damage Response. Cell Rep. 2020 Jun. 2; 31(9):107723. doi: 10.1016/j.celrep.2020.107723. PMID: 32492431; and (3) Swartjes T, Staals R H J, van der Oost J. Editor's cut: DNA cleavage by CRISPR RNA-guided nucleases Cas9 and Cas12a. Biochem Soc Trans. 2020 Feb. 28; 48(1):207-219. doi: 10.1042/BST20190563. PMID: 31872209; PMCID: PMC7054755: (4) Gaillochet C, Peña Fernãndez, A, Goossens V. D'Halluin K, Drozdzecki A, Shafie M, Van Duyse J, Van Isterdael G, Gonzalez C, Vermeersch M, De Saeger J, Develtere W, Audenaert D, De Vleesschauwer D, Meulewaeter F, Jacobs T B. Systematic optimization of Cas12a base editors in wheat and maize using the ITER platform. Genome Biol. 2023 Jan. 13; 24(1):6. doi: 10.1186/s13059-022-02836-2. PMID: 36639800; PMCID: PMC9838060; each of which are incorporated herein by reference in their entireties.

Base editing was first described in Komor et al., “Programmable editing of a target base in genomic DNA without double-stranded DNA cleavage,” Nature, May 19, 2016, 533 (7603); pp. 420-424 in the form of cytosine base editors or CBEs followed by the disclosure of Gaudelli et al., “Programmable base editing of A-T to G-C in genomic DNA without DNA cleavage,” Nature, Vol. 551, pp. 464-471 describing adenine base editors or ABEs. Subsequently, base editing has been described in numerous scientific publications, including, but not limited to (i) Kim JS. Precision genome engineering through adenine and cytosine base editing. Nat Plants. 2018 March; 4(3):148-151. doi: 10.1038/s41477-018-0115-z. Epub 2018 Feb. 26. PMID: 29483683.; (ii) Wei Y, Zhang X H, Li DL. The “new favorite” of gene editing technology-single base editors. Yi Chuan. 2017 Dec. 20; 39(12):1115-1121. doi: 10.16288/j.yczz.17-389. PMID: 29258982; (iii) Tang J, Lee T, Sun T. Single-nucleotide editing: From principle, optimization to application. Hum Mutat. 2019 December; 40(12):2171-2183. doi: 10.1002/humu.23819. Epub 2019 Sep. 15. PMID: 31131955; PMCID: PMC6874907; (iv) Grunewald J, Zhou R, Lareau C A, Garcia S P, Iyer S, Miller B R, Langner L M, Hsu J Y, Aryee M J, Joung J K. A dual-deaminase CRISPR base editor enables concurrent adenine and cytosine editing. Nat Biotechnol. 2020 July; 38(7):861-864. doi: 10.1038/s41587-020-0535-y. Epub 2020 Jun. 1. PMID: 32483364; PMCID: PMC7723518; (v) Sakata R C, Ishiguro S, Mori H, Tanaka M, Tatsuno K, Ueda H, Yamamoto S, Seki M, Masuyama N, Nishida K, Nishimasu H, Arakawa K, Kondo A, Nureki O, Tomita M, Aburatani H, Yachie N. Base editors for simultaneous introduction of C-to-T and A-to-G mutations. Nat Biotechnol. 2020 July; 38(7):865-869. doi: 10.1038/s41587-020-0509-0. Epub 2020 Jun. 2. Erratum in: Nat Biotechnol. 2020 Jun. 5; PMID: 32483365; (vi) Fan J, Ding Y, Ren C, Song Z, Yuan J, Chen Q, Du C, Li C, Wang X, Shu W. Cytosine and adenine deaminase base-editors induce broad and nonspecific changes in gene expression and splicing. Commun Biol. 2021 Jul. 16; 4(1):882. doi: 10.1038/s42003-021-02406-5. PMID: 34272468; PMCID: PMC8285404; (vii) Zhang S, Yuan B, Cao J, Song L, Chen J, Qiu J, Qiu Z, Zhao X M, Chen J, Cheng T L. TadA orthologs enable both cytosine and adenine editing of base editors. Nat Commun. 2023 Jan. 26; 14(1):414. doi: 10.1038/s41467-023-36003-3. PMID: 36702837; PMCID: PMC988000; and (viii) Zhang S, Song L, Yuan B, Zhang C, Cao J, Chen J, Qiu J, Tai Y, Chen J, Qiu Z, Zhao X M, Cheng T L. TadA reprogramming to generate potent miniature base editors with high precision. Nat Commun. 2023 Jan. 26; 14(1):413. doi: 10.1038/s41467-023-36004-2. PMID: 36702845; PMCID: PMC987999, each of which are incorporated herein by reference in their entireties.

Amino acid and nucleotide sequences of base editors, including adenosine base editors, cytidine base editors, and others are readily available in the art. For example, exemplary base editors that may be delivered using the LNP compositions described herein can be found in the following published patent applications, each of their contents (including any and all biological sequences) are incorporated herein by reference:

| | |
|---|---|
| US 2023/0021641 A1 | CAS9 VARIANTS HAVING NON-CANONICAL PAM SPECIFICITIES |
| | AND USES THEREOF |
| U.S. Pat. No. 11,542,496 B2 | Cytosine to guanine base editor |
| U.S. Pat. No. 11,542,509 B2 | Incorporation of unnatural amino acids into proteins using base editing |
| US 2022/0315906 A1 | BASE EDITORS WITH DIVERSIFIED TARGETING SCOPE |
| US 2022/0282275 A1 | G-TO-T BASE EDITORS AND USES THEREOF |
| US 2022/0249697 A1 | AAV DELIVERY OF NUCLEOBASE EDITORS |

In some embodiments, the LNP cargo comprises a base editing system or a polynucleotide encoding a CRISPR-Cas base editing system. In some embodiments, the cargo comprises a component of a base editing system or a polynucleotide encoding a component of a base editing system.

Base editing does not require double-stranded DNA breaks or a DNA donor template. In some embodiments, base editing comprises creating an SSB in a target double-stranded DNA sequence and then converting a nucleobase. In some embodiments, the nucleobase conversion is an adenosine to a guanine. In some embodiments, the nucleobase conversion is a thymine to a cytosine. In some embodiments, the nucleobase conversion is a cytosine to a thymine. In some embodiments, the nucleobase conversion is a guanine to an adenosine. In some embodiments, the nucleobase conversion is an adenosine to inosine. In some embodiments, the nucleobase conversion is a cytosine to uracil.

A base editing system comprises a base editor which can convert a nucleobase. The base editor (“BE”) comprises a partially inactive Cas protein which is connected to a deaminase that precisely and permanently edits a target nucleobase in a polynucleotide sequence. A base editor comprises a polynucleotide programmable nucleotide binding domain and a nucleobase editing domain (e.g., adenosine deaminase or cytosine deaminase). In some embodiments, the partially inactive Cas protein is a Cas nickase. In some embodiments, the partially inactive Cas protein is a Cas9 nickase (also referred to as “nCas9”).

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleobase and bases of the target polynucleotide sequence) and thereby localize the nucleobase editor to the target polynucleotide sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In certain embodiments, polynucleotide programmable nucleotide binding domains also include nucleobase programmable proteins that bind RNA. In certain embodiments, the polynucleotide programmable nucleotide binding domain can be associated with a nucleobase that guides the polynucleotide programmable nucleotide binding domain to an RNA.

In some embodiments, the LNP-deliverable base editors may comprise a deaminase domain that is a cytidine deaminase domain. A cytidine deaminase domain may also be referred to interchangeably as a cytosine deaminase domain. In some embodiments, the cytidine deaminase catalyzes the hydrolytic deamination of cytidine (C) or deoxycytidine (dC) to uridine (U) or deoxyuridine (dU), respectively. In some embodiments, the cytidine deaminase domain catalyzes the hydrolytic deamination of cytosine (C) to uracil (U). In some embodiments, the cytidine deaminase catalyzes the hydrolytic deamination of cytidine or cytosine in deoxyribonucleic acid (DNA). Without wishing to be bound by any particular theory, fusion proteins comprising a cytidine deaminase are useful inter alia for targeted editing, referred to herein as “base editing,” of nucleic acid sequences in vitro and in vivo.

One exemplary suitable type of cytidine deaminase is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner (see, e.g., Conticello S G. The AID/APOBEC family of nucleic acid mutators. Genome Biol. 2008; 9(6):229). One family member, activation-induced cytidine deaminase (AID), is responsible for the maturation of antibodies by converting cytosines in ssDNA to uracils in a transcription-dependent, strand-biased fashion (see, e.g., Reynaud C A, et al. What role for AID: mutator, or assembler of the immunoglobulin mutasome, Nat Immunol. 2003; 4(7):631-638). The apolipoprotein B editing complex 3 (APOBEC3) enzyme provides protection to human cells against a certain IIIV-1 strain via the deamination of cytosines in reverse-transcribed viral ssDNA (see, e.g., Bhagwat A S. DNA-cytosine deaminases: from antibody maturation to antiviral defense. DNA Repair (Amst). 2004; 3(1):85-89).

Some aspects of this disclosure relate to the recognition that the activity of cytidine deaminase enzymes such as APOBEC enzymes can be directed to a specific site in genomic DNA.

Without wishing to be bound by any particular theory, advantages of using a nucleic acid programmable binding protein (e.g., a Cas9 domain) as a recognition agent include (1) the sequence specificity of nucleic acid programmable binding protein (e.g., a Cas9 domain) can be easily altered by simply changing the sgRNA sequence; and (2) the nucleic acid programmable binding protein (e.g., a Cas9 domain) may bind to its target sequence by denaturing the dsDNA, resulting in a stretch of DNA that is single-stranded and therefore a viable substrate for the deaminase. It should be understood that other catalytic domains of napDNAbps, or catalytic domains from other nucleic acid editing proteins, can also be used to generate fusion proteins with Cas9, and that the disclosure is not limited in this regard.

In some embodiments, the cytidine deaminase is an apolipoprotein B mRNA-editing complex (APOBEC) family deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a vertebrate cytidine deaminase. In some embodiments, the cytidine deaminase is an invertebrate cytidine deaminase. In some embodiments, the cytidine deaminase is a human, chimpanzee, gorilla, monkey, cow, dog, rat, or mouse deaminase. In some embodiments, the cytidine deaminase is a human cytidine deaminase. In some embodiments, the cytidine deaminase is a rat cytidine deaminase, e.g., rAPOBEC1.

In some embodiments, the nucleic acid editing domain is at least 80%, at least 85%, at least 90%, at least 92%, at least 95%, at least 96%, at least 97%, at least 98%, at least 99%, or at least 99.5% identical to any of the cytidine deaminase domain examples above.

In other embodiments, the LNP-deliverable base editors may comprise a deaminase domain that is an adenosine deaminase domain.

The disclosure provides fusion proteins that comprise one or more adenosine deaminases. In some aspects, such fusion proteins are capable of deaminating adenosine in a nucleic acid sequence (e.g., DNA or RNA). As one example, any of the fusion proteins provided herein may be base editors, (e.g., adenine base editors). Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the fusion proteins may comprise 2, 3, 4 or 5 adenosine deaminases. In some embodiments, any of the fusion proteins provided herein comprise two adenosine deaminases. Exemplary, non-limiting, embodiments of adenosine deaminases are provided herein. It should be appreciated that the mutations provided herein (e.g., mutations in ecTadA) may be applied to adenosine deaminases in other adenosine base editors, for example those provided in U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. W O 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, all of which are incorporated herein by reference in their entireties.

In some embodiments, any of the adenosine deaminases provided herein is capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

Any two or more of the adenosine deaminases described herein may be connected to one another (e.g. by a linker) within an adenosine deaminase domain of the fusion proteins provided herein. For instance, the fusion proteins provided herein may contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. In some embodiments, the fusion protein comprises two adenosine deaminases (e.g., a first adenosine deaminase and a second adenosine deaminase). In some embodiments, the fusion protein comprises a first adenosine deaminase and a second adenosine deaminase. In some embodiments, the first adenosine deaminase is N-terminal to the second adenosine deaminase in the fusion protein. In some embodiments, the first adenosine deaminase is C-terminal to the second adenosine deaminase in the fusion protein. In some embodiments, the first adenosine deaminase and the second deaminase are fused directly or via a linker.

In some embodiments, the base editor comprises a deaminase enzyme. In some embodiments, the base editor comprises a cytidine deaminase. In some embodiments, the base editor comprises a Cas9 protein fused to a cytidine deaminase enzyme. In some embodiments, the base editor comprises an adenosine deaminase. In some embodiments, the base editor comprises a Cas9 protein fused to an adenosine deaminase enzyme.

In some embodiments, the base editing system comprises an uracil glycosylase inhibitor. In some embodiments, the base editing system comprises a Cas9 protein fused to an uracil glycosylase inhibitor. In some embodiments, the cargo comprises an uracil glycosylase inhibitor or a polynucleotide encoding an uracil glycosylase inhibitor. In some embodiments, the cargo comprises a Cas9 protein fused to an uracil glycosylase inhibitor or a polynucleotide encoding a Cas9 protein fused to an uracil glycosylase inhibitor.

A variety of nucleobase modifying enzymes are suitable for use in the nucleobase systems disclosed herein. In some embodiments, the nucleobase modifying enzyme is a RNA base editor. In some embodiments, the RNA base editor can be a cytidine deaminase, which converts cytidine into uridine. Non-limiting examples of cytidine deaminases include cytidine deaminase 1 (CDA1), cytidine deaminase 2 (CDA2), activation-induced cytidine deaminase (AICDA), apolipoprotein B mRNA-editing complex (APOBEC) family cytidine deaminase (e.g., APOBEC1, APOBEC2, APOBEC3A, APOBEC3B, APOBEC3C, APOBEC3D/E, APOBEC3F, APOBEC3G, APOBEC3H, APOBEC4), APOBEC1 complementation factor/APOBEC1 stimulating factor (ACF1/ASF) cytidine deaminase, cytosine deaminase acting on RNA (CDAR), bacterial long isoform cytidine deaminase (CDDL), and cytosine deaminase acting on tRNA (CDAT). In other embodiments, the RNA base editor can be an adenosine deaminase, which converts adenosine into inosine, which is read by polymerase enzymes as guanosine. In certain embodiments, adenosine deaminases include tRNA adenine deaminase, adenosine deaminase, adenosine deaminase acting on RNA (ADAR), and adenosine deaminase acting on tRNA (ADAT).

In some embodiments, in the nucleobase editing systems disclosed herein, the Cas effector may associate with one or more functional domains (e.g., via fusion protein or suitable linkers). In some embodiments, the effector domain comprises one or more cytidine or nucleotide deaminases that mediate editing of via hydrolytic deamination. In certain embodiments, the effector domain comprises the adenosine deaminase acting on RNA (ADAR) family of enzymes. In certain embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

In some embodiments, the cytidine deaminase is a human, rat or lamprey cytidine deaminase. In some embodiments, the cytidine deaminase is an apolipoprotein B mRNA-editing complex (APOBEC) family deaminase, an activation-induced deaminase (AID), or a cytidine deaminase 1 (CDA1).

In certain embodiments, the adenosine deaminase is adenosine deaminase acting on RNA (ADAR). In certain embodiments, the ADAR is ADAR (ADAR1), ADARB1 (ADAR2) or ADARB2 (ADAR3) (see, e.g., Savva et al. Genon. Biol. 2012, 13(12):252).

In some embodiments, the gene editing system comprises AID/APOBEC (apolipoprotein B editing complex) family of enzymes deaminates cytidine to uridine, leading to mutations in RNA and DNA.

In some embodiments, the nucleobase editing system comprises ADAR and an antisense oligonucleotide. In certain embodiments, the antisense oligonucleotide is chemically optimized antisense oligonucleotide. In certain embodiments, the antisense oligonucleotide is administered for the nucleobase editing, wherein the antisense oligonucleotide activates human endogenous ADAR for nucleobase editing. Such ADAR and antisense oligonucleotide editing system provides a safer site-directed RNA editing with low off-target effect. See, e.g., Merkle et al., Nature Biotechnology, 2019, 37, 133-138.

Any of the above base editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### Prime Editors

In various embodiments, the herein disclosed LNPs may contain a prime editing system or components thereof and which may be used to conduct prime editing of target nucleic acid sequences in cells, tissues, and organs in an ex vivo or in vivo manner.

Prime editing technology is a gene editing technology that can make targeted insertions, deletions, and all transversion and transition point mutations in a target genome. Without wishing to be bound by any particular theory, the prime editing process may search and replace endogenous sequences in a target polynucleotide. The spacer sequence of a prime editing guide RNA (“PEgRNA” or “pegRNA”) recognizes and anneals with a search target sequence in a target strand of a double stranded target polynucleotide, e.g., a double stranded target DNA. A prime editing complex may generate a nick in the target DNA on the edit strand which is the complementary strand of the target strand. The prime editing complex may then use a free 3′ end formed at the nick site of the edit strand to initiate DNA synthesis, where a “primer binding site sequence” (PBS) of the PEgRNA complexes with the free 3′ end, and a single stranded DNA is synthesized (by reverse transcriptase) using an editing template of the PEgRNA as a template. As used herein, a “primer binding site” is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site.

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

The editing template may comprise one or more intended nucleotide edits compared to the endogenous double stranded target DNA sequence. Accordingly, the newly synthesized single stranded DNA also comprises the nucleotide edit(s) encoded by the editing template. Through removal of the editing target sequence on the edit strand of the double stranded target DNA and DNA repair mechanism, the newly synthesized single stranded DNA replaces the editing target sequence, and the desired nucleotide edit(s) are incorporated into the double stranded target DNA.

Prime editing was first described in Anzalone et al., “Search-and-replace genome editing without double-strand breaks or donor DNA,” Nature, December 2019, 576 (7789): pp. 149-157, which is incorporated herein in its entirety. Prime editing has subsequently been described and detailed in numerous follow-on publications, including, for example, (i) Liu et al., “Prime editing: a search and replace tool with versatile base changes,” Yi Chuan, Nov. 20, 2022, 44(11): 993-1008; (ii) Lu C et al., “Prime Editing: An All-Rounder for Genome Editing. Int J Mol Sci. 2022 Aug. 30; 23(17):9862; (iii) Velimirovic M, Zanetti L C, Shen M W, Fife J D, Lin L, Cha M, Akinci E, Barnum D. Yu T, Sherwood R I. Peptide fusion improves prime editing efficiency. Nat Commun. 2022 Jun. 18; 13(1):3512. doi: 10.1038/s41467-022-31270-y. PMID: 35717416; PMCID: PMC9206660; (iv) Velimirovic M, Zanetti L C, Shen M W, Fife J D, Lin L, Cha M, Akinci E, Barnum D, Yu T, Sherwood R I. Peptide fusion improves prime editing efficiency. Nat Commun. 2022 Jun. 18; 13(1):3512. doi: 10.1038/s41467-022-31270-y. PMID: 35717416; PMCID: PMC9206660; (v) Habib O, Habib G, Hwang G H, Bae S. Comprehensive analysis of prime editing outcomes in human embryonic stem cells. Nucleic Acids Res. 2022 Jan. 25; 50(2):1187-1197. doi: 10.1093/nar/gkabl295. PMID: 35018468; PMCID: PMC8789035; (vi) Marzec M, Breszewska-Zalewska A, IIensel G. Prime Editing: A New Way for Genome Editing. Trends Cell Biol. 2020 April; 30(4):257-259. doi: 10.1016/j.tcb.2020.01.004. Epub 2020 Jan. 27. PMID: 32001098; (vii) Tao R, Wang Y, Jiao Y, Hu Y, Li L, Jiang L, Zhou L, Qu J, Chen Q, Yao S. Bi-PE: bi-directional priming improves CRISPR/Cas9 prime editing in mammalian cells. Nucleic Acids Res. 2022 Jun. 24; 50(11):6423-6434. doi: 10.1093/nar/gkac506. PMID: 35687127; PMCID: PMC9226529; (viii) Nelson J W, Randolph P B, Shen S P, Everette K A, Chen P J, Anzalone A V, An M, Newby G A, Chen J C, Hsu A, Liu D R. Engineered pegRNAs improve prime editing efficiency. Nat Biotechnol. 2022 March; 40(3):402-410. doi: 10.1038/s41587-021-01039-7. Epub 2021 Oct. 4. Erratum in: Nat Biotechnol. 2021 Dec. 8; PMID: 34608327; PMCID: PMC8930418; (ix) Doman J L, Sousa A A, Randolph P B, Chen P J, Liu D R. Designing and executing prime editing experiments in mammalian cells. Nat Protoc. 2022 November; 17(11):2431-2468. doi: 10.1038/s41596-022-00724-4. Epub 2022 Aug. 8. PMID: 35941224; PMCID: PMC9799714; (x) Jiao Y, Zhou L, Tao R, Wang Y, Hu Y, Jiang L, Li L, Yao S. Randon-P E: an efficient integration of random sequences into mammalian genome by prime editing. Mol Biomed. 2021 Nov. 18; 2(1):36. doi: 10.1186/s43556-021-00057-w. PMID: 35006470; PMCID: PMC8607425; and (xi) Awan M J A, Ali Z, Amin I, Mansoor S. Twin prime editor: seamless repair without damage. Trends Biotechnol. 2022 April; 40(4):374-376. doi: 10.1016/j.tibtech.2022.01.013. Epub 2022 Feb. 10. PMID: 35153078, all of which are incorporated herein by reference.

In addition, prime editing has been described and disclosed in numerous published patent applications, each of which their entire contents, amino acid sequences, nucleotide sequences, and all disclosures therein are incorporated herein by reference in their entireties:

| Publication No. | Publication Date | Title |
|---|---|---|
| WO 2023/015309 A2 | Feb. 9, 2023 | IMPROVED PRIME EDITORS AND METHODS |
| | | OF USE |
| WO 2023/004439 A2 | Jan. 26, 2023 | GENOME EDITING COMPOSITIONS AND |
| | | METHODS FOR TREATMENT OF CHRONIC |
| | | GRANULOMATOUS DISEASE |
| WO 2023/288332 A2 | Jan. 19, 2023 | GENOME EDITING COMPOSITIONS AND |
| | | METHODS FOR TREATMENT OF WILSON'S |
| | | DISEASE |
| WO 2023/283092 A1 | Jan. 12, 2023 | COMPOSITIONS AND METHODS FOR |
| | | EFFICIENT GENOME EDITING |
| WO 2023/283246 A1 | Jan. 12, 2023 | MODULAR PRIME EDITOR SYSTEMS FOR |
| | | GENOME ENGINEERING |
| WO 2022/256714 A3 | Jan. 12, 2023 | GENOME EDITING COMPOSITIONS AND |
| | | METHODS FOR TREATMENT OF WILSON'S |
| | | DISEASE |
| EP 4107273 A1 | Dec. 28, 2022 | PRIME EDITING TECHNOLOGY FOR PLANT |
| | | GENOME ENGINEERING |
| WO 2022/256714 A2 | Dec. 8, 2022 | GENOME EDITING COMPOSITIONS AND |
| | | METHODS FOR TREATMENT OF WILSON'S |
| | | DISEASE |
| WO 2022/234051 A1 | Nov. 10, 2022 | SPLIT PRIME EDITING ENZYME |
| US 2022/0356469 A1 | Nov. 10, 2022 | METHODS AND COMPOSITIONS FOR EDITING |
| | | NUCLEOTIDE SEQUENCES METHODS AND |
| | | COMPOSITIONS FOR EDITING NUCLEOTIDE |
| | | SEQUENCES |
| WO 2022/206352 A1 | Oct. 6, 2022 | PRIME EDITING TOOL, FUSION RNA, AND |
| | | USE THEREOF |
| WO 2022/212926 A1 | Oct. 6, 2022 | METHODS AND COMPOSITIONS FOR EDITING |
| | | NUCLEOTIDE SEQUENCES |
| WO 2022/204476 A1 | Sep. 29, 2022 | NUCLEOTIDE EDITING TO REFRAME DMD |
| | | TRANSCRIPTS BY BASE EDITING AND PRIME |
| | | EDITING |
| WO 2022/203905 A1 | Sep. 29, 2022 | PRIME EDITING-BASED SIMULTANEOUS |
| | | GENOMIC DELETION AND INSERTION |
| U.S. Pat. No. 11,447,770 B1 | Sep. 20, 2022 | Methods and compositions for prime editing |
| | | nucleotide sequences |
| WO 2022/174829 A1 | Aug. 25, 2022 | EDITING OF DOUBLE-STRANDED DNA WITH |
| | | RELAXED PAM REQUIREMENT FIELD OF THE |
| | | DISCLOSURE |
| WO 2022/170058 A1 | Aug. 11, 2022 | PRIME EDITOR SYSTEM FOR IN VIVO |
| | | GENOME EDITING |
| WO 2022/169235 A1 | Aug. 11, 2022 | PRIME EDITING COMPOSITION WITH |
| | | IMPROVED EDITING EFFICIENCY |
| WO 2022/150790 A3 | Aug. 11, 2022 | PRIME EDITOR VARIANTS, CONSTRUCTS, |
| | | AND METHODS FOR ENHANCING PRIME |
| | | EDITING EFFICIENCY AND PRECISION |
| WO 2022/149166 A1 | Jul. 14, 2022 | A COCKTAIL FORMULATION FOR SELECTIVE |
| | | ENRICHMENT OF GENE-MODIFIED CELLS |
| WO 2022/150790 A2 | Jul. 14, 2022 | PRIME EDITOR VARIANTS, CONSTRUCTS, |
| | | AND METHODS FOR ENHANCING PRIME |
| | | EDITING EFFICIENCY AND PRECISION |
| U.S. Pat. No. 11,384,353 B2 | Jul. 12, 2022 | Inhibition of unintended mutations in gene editing |
| WO 2022/067130 A3 | Jun. 23, 2022 | PRIME EDITING GUIDE RNAS, |
| | | COMPOSITIONS THEREOF, AND METHODS OF |
| | | USING THE SAME |
| WO 2022/114815 A1 | Jun. 2, 2022 | COMPOSITION FOR PRIME EDITING |
| | | COMPRISING TRANS-SPLICING ADENO- |
| | | ASSOCIATED VIRUS VECTOR |
| WO 2022/100662 A1 | May 19, 2022 | GENOMIC EDITING OF IMPROVED |
| | | EFFICIENCY AND ACCURACY |
| WO 2022/098765 A1 | May 12, 2022 | SPLIT PRIME EDITING PLATFORMS |
| WO 2022/098885 A1 | May 12, 2022 | PRECISE GENOME DELETION AND |
| | | REPLACEMENT METHOD BASED ON PRIME |
| | | EDITING |
| WO 2022/071745 A1 | Apr. 7, 2022 | PRIME EDITING USING HIV REVERSE |
| | | TRANSCRIPTASE AND CAS9 OR VARIANT |
| | | THEREOF |
| WO 2022/067130 A2 | Mar. 31, 2022 | PRIME EDITING GUIDE RNAS, |
| | | COMPOSITIONS THEREOF, AND METHODS OF |
| | | USING THE SAME |
| WO 2022/065689 A1 | Mar. 31, 2022 | PRIME EDITING-BASED GENE EDITING |
| | | COMPOSITION WITH ENHANCED EDITING |
| | | EFFICIENCY AND USE THEREOF |
| US 2022/0064626 A1 | Mar. 3, 2022 | INHIBITION OF UNINTENDED MUTATIONS IN |
| | | GENE EDITING |
| WO 2022/032085 A1 | Feb. 10, 2022 | TARGETED SEQUENCE INSERTION |
| | | COMPOSITIONS AND METHODS |
| WO 2022/025623 A1 | Feb. 3, 2022 | SYSTEM AND METHOD FOR PRIME EDITING |
| | | EFFICIENCY PREDICTION USING DEEP |
| | | LEARNING |
| WO 2021/226558 A8 | Jan. 13, 2022 | METHODS AND COMPOSITIONS FOR |
| | | SIMULTANEOUS EDITING OF BOTH STRANDS |
| | | OF A TARGET DOUBLE-STRANDED |
| | | NUCLEOTIDE SEQUENCE |
| WO 2021/243289 A1 | Dec. 2, 2021 | SYSTEMS AND METHODS FOR STABLE AND |
| | | HERITABLE ALTERATION BY PRECISION |
| | | EDITING (SHAPE) |
| WO 2021/226558 A1 | Nov. 11, 2021 | METHODS AND COMPOSITIONS FOR |
| | | SIMULTANEOUS EDITING OF BOTH STRANDS |
| | | OF A TARGET DOUBLE-STRANDED |
| | | NUCLEOTIDE SEQUENCE |
| WO 2021/215897 A1 | Oct. 28, 2021 | GENOME EDITION USING CAS9 OR CAS9 |
| | | VARIANT |
| WO 2021/215827 A1 | Oct. 28, 2021 | GENOME EDITING USING CAS9 OR CAS9 |
| | | VARIANT |
| WO 2020/191248 A8 | Oct. 21, 2021 | METHODS AND COMPOSITIONS FOR EDITING |
| | | NUCLEOTIDE SEQUENCES |
| WO 2020/191234 A8 | Oct. 21, 2021 | METHODS AND COMPOSITIONS FOR EDITING |
| | | NUCLEOTIDE SEQUENCES |
| WO 2021/165508 A1 | Aug. 26, 2021 | PRIME EDITING TECHNOLOGY FOR PLANT |
| | | GENOME ENGINEERING |
| WO 2021/138469 A1 | Jul. 8, 2021 | GENOME EDITING USING REVERSE |
| | | TRANSCRIPTASE ENABLED AND FULLY |
| | | ACTIVE CRISPR COMPLEXES |
| WO 2021/092204 A1 | May 14, 2021 | METHODS AND COMPOSITIONS FOR |
| | | NUCLEIC ACID-GUIDED NUCLEASE CELL |
| | | TARGETING SCREEN |
| WO 2021/076876 A1 | Apr. 22, 2021 | GENOTYPING EDITED MICROBIAL STRAINS |
| WO 2021/072328 A1 | Apr. 15, 2021 | METHODS AND COMPOSITIONS FOR PRIME |
| | | EDITING RNA |
| WO 2020/191153 A8 | Dec. 30, 2020 | METHODS AND COMPOSITIONS FOR EDITING |
| | | NUCLEOTIDE SEQUENCES |
| WO 2020/191153 A3 | Dec. 10, 2020 | METHODS AND COMPOSITIONS FOR EDITING |
| | | NUCLEOTIDE SEQUENCES |
| WO 2020/191153 A9 | Nov. 12, 2020 | METHODS AND COMPOSITIONS FOR EDITING |
| | | NUCLEOTIDE SEQUENCES |
| WO 2020/191171 A9 | Oct. 29, 2020 | METHODS AND COMPOSITIONS FOR EDITING |
| | | NUCLEOTIDE SEQUENCES |
| WO 2020/191248 A1 | Sep. 24, 2020 | METHOD AND COMPOSITIONS FOR EDITING |
| | | NUCLEOTIDE SEQUENCES |
| WO 2020/191239 A1 | Sep. 24, 2020 | METIIODS AND COMPOSITIONS FOR EDITING |
| | | NUCLEOTIDE SEQUENCES |
| WO 2020/191153 A2 | Sep. 24, 2020 | METHODS AND COMPOSITIONS FOR EDITING |
| | | NUCLEOTIDE SEQUENCES |
| WO 2020/191246 A1 | Sep. 24, 2020 | METHODS AND COMPOSITIONS FOR EDITING |
| | | NUCLEOTIDE SEQUENCES |
| WO 2020/191249 A1 | Sep. 24, 2020 | METHODS AND COMPOSITIONS FOR EDITING |
| | | NUCLEOTIDE SEQUENCES |
| WO 2020/191233 A1 | Sep. 24, 2020 | METHODS AND COMPOSITIONS FOR EDITING |
| | | NUCLEOTIDE SEQUENCES |
| WO 2020/191243 A1 | Sep. 24, 2020 | METHODS AND COMPOSITIONS FOR EDITING |
| | | NUCLEOTIDE SEQUENCES |
| WO 2020/191234 A1 | Sep. 24, 2020 | METHODS AND COMPOSITIONS FOR EDITING |
| | | NUCLEOTIDE SEQUENCES |
| WO 2020/191245 A1 | Sep. 24, 2020 | METHODS AND COMPOSITIONS FOR EDITING |
| | | NUCLEOTIDE SEQUENCES |
| WO 2020/191242 A1 | Sep. 24, 2020 | METHODS AND COMPOSITIONS FOR EDITING |
| | | NUCLEOTIDE SEQUENCES |
| WO 2020/191171 A1 | Sep. 24, 2020 | METHODS AND COMPOSITIONS FOR EDITING |
| | | NUCLEOTIDE SEQUENCES |
| WO 2020/191241 A1 | Sep. 24, 2020 | METHODS AND COMPOSITIONS FOR EDITING |
| | | NUCLEOTIDE SEQUENCES |
| WO 2020/156575 A1 | Aug. 6, 2020 | INHIBITION OF UNINTENDED MUTATIONS IN |
| | | GENE EDITING |
| U.S. Pat. No. 10,189,831 B2 | Jan. 29, 2019 | Non-nucleoside reverse transcriptase inhibitors |
| WO 2019/014564 A1 | Jan. 17, 2019 | SYSTEMS AND METHODS FOR TARGETED |
| | | INTEGRATION AND GENOME EDITING AND |
| | | DETECTION THEREOF USING INTEGRATED |
| | | PRIMING SITES |
| U.S. Pat. No. 10,150,955 B2 | Dec. 11, 2018 | Stabilized reverse transcriptase fusion proteins |
| WO 2018/049168 A1 | Mar. 15, 2018 | HIGH-THROUGHPUT PRECISION GENOME |
| | | EDITING |
| U.S. Pat. No. 9,783,791 B2 | Oct. 10, 2017 | Mutant reverse transcriptase and methods of use |
| U.S. Pat. No. 9,458,484 B2 | Oct. 4, 2016 | Reverse transcriptase mixtures with improved |
| | | storage stability |
| WO 2023/070110 A2 | Apr. 27, 2023 | GENOME EDITING COMPOSITIONS AND |
| | | METHODS FOR TREATMENT OF RETINITIS |
| | | PIGMENTOSA |
| US 2023/0059368 A1 | Feb. 23, 2023 | POLYNUCLEOTIDE EDITORS AND METHODS |
| | | OF USING THE SAME |
| WO 2023/086558 A1 | May 19, 2023 | GENOME EDITING COMPOSITIONS AND |
| | | METHODS FOR TREATMENT OF FRAGILE X |
| | | SYNDROME |
| WO 2023/288332 A3 | Feb. 23, 2023 | GENOME EDITING COMPOSITIONS AND |
| | | METHODS FOR TREATMENT OF WILSON'S |
| | | DISEASE |
| WO 2023/192655 A2 | Oct. 5, 2023 | METHODS AND COMPOSITIONS FOR EDITING |
| | | NUCLEOTIDE SEQUENCES |
| WO 2022/256714 A2 | Dec. 8, 2022 | GENOME EDITING COMPOSITIONS AND |
| | | METHODS FOR TREATMENT OF WILSON'S |
| | | DISEASE |
| WO 2023/283092 A1 | Jan. 12, 2023 | COMPOSITIONS AND METHODS FOR |
| | | EFFICIENT GENOME EDITING |
| WO 2023/070062 A3 | May 25, 2023 | GENOME EDITING COMPOSITIONS AND |
| | | METHODS FOR TREATMENT OF USHER |
| | | SYNDROME TYPE 3 |
| WO 2023/081787 A3 | Sep. 28, 2023 | GENOME EDITING COMPOSITIONS AND |
| | | METHODS FOR TREATMENT OF FANCONI |
| | | ANEMIA |
| WO 2023/086389 A1 | May 19, 2023 | GENOME EDITING COMPOSITIONS AND |
| | | METHODS FOR TREATMENT OF |
| | | AMYOTROPHIC LATERAL SCLEROSIS |
| WO 2023/096847 A2 | Jun. 1, 2023 | METHODS AND COMPOSITIONS FOR |
| | | INHIBITING MISMATCH REPAIR |
| WO 2023/081426 A1 | May 11, 2023 | GENOME EDITING COMPOSITIONS AND |
| | | METHODS FOR TREATMENT OF |
| | | FRIEDREICH'S ATAXIA |
| WO 2023/015318 A2 | Feb. 9, 2023 | GENOME EDITING COMPOSITIONS AND |
| | | METHODS FOR TREATMENT OF CYSTIC |
| | | FIBROSIS |
| WO 2022/212926 A1 | Oct. 6, 2022 | METHODS AND COMPOSITIONS FOR EDITING |
| | | NUCLEOTIDE SEQUENCES |
| WO 2023/028180 A3 | Aug. 24, 2023 | GENOME EDITING COMPOSITIONS AND |
| | | METHODS FOR TREATMENT OF |
| | | RETINOPATIIY |
| WO 2023/015014 A1 | Feb. 9, 2023 | GENOME EDITING COMPOSITIONS AND |
| | | METHODS FOR TREATMENT OF MYOTONIC |
| | | DYSTROPHY |
| WO 2023/086842 A1 | May 19, 2023 | GENOME EDITING COMPOSITIONS AND |
| | | METHODS FOR TREATMENT OF FUCHS |
| | | ENDOTHELIAL CORNEAL DYSTROPHY |
| WO 2023/096992 A1 | Jun. 1, 2023 | GENOME EDITING COMPOSITIONS AND |
| | | METHODS FOR TREATMENT OF GLYCOGEN |
| | | STORAGE DISEASE TYPE 1B |
| WO 2023/096977 A2 | Jun. 1, 2023 | MODIFIED PRIME EDITING GUIDE RNAS |

In some embodiments, the cargo comprises a prime editing system or a polynucleotide encoding a prime editing system. In some embodiments, the cargo comprises a component of a prime editing system or a polynucleotide encoding a component of a prime editing system.

Prime editing is a versatile and precise genome editing method that directly writes new genetic information into a specified DNA site using a catalytically impaired Cas fused to an engineered reverse transcriptase, also referred to as a prime editor, which is programmable using a prime editing guide RNA (“pegRNA”) that both specifies the target site and encodes the desired edit (see, e.g., Anzalone et al., Nature 2019). Prime editing bypasses the need for DNA donor templates by using a prime editor having nickase or catalytically impaired enzymatic activity.

A prime editing system comprises a prime editor. The prime editor (“PE”) comprises a catalytically impaired Cas protein fused to an engineered reverse transcriptase which can precisely and permanently edit one or more target nucleobases in a target polynucleotide.

In some embodiments, the prime editor comprises an engineered Moloney murine leukemia virus (“M-MLV”) reverse transcriptase (“RT”) fused to a Cas-H840A nickase (called “PE2”). In some embodiments, the prime editor comprises an engineered M-MLV RT fused to a Cas9-H840A nickase.

In some embodiments, the prime editor comprises an engineered M-MLV RT fused to a Streptococcus pyogenes Cas9 (spCas9)-H840A nickase. P E modifications include increased PAM flexibility to increase the utility of PE2 editing, expanding the coverage of targetable pathogenic variants in the ClinVar database that can now be prime edited to 94.4%.

In some embodiments, the prime editing system further comprises a prime editing guide RNA (“pegRNA”). In some embodiments, the cargo comprises a pegRNA or a polynucleotide encoding a pegRNA. [00800]pegRNAs may be designed and synthesized using methods, software, and commercial sources which are well known to those having ordinary skill in the art such that guide RNAs for any given naspDBP or prime editor may be obtained without undue experimentation.

Reference may be made to the following references providing information and tools for the design, synthesis, modification, and structural configuration of pegRNAs, and guide RNAs in general: (1) Mohr S E, Hu Y, Ewen-Campen B, Housden B E, Viswanatha R, Perrimon N. CRISPR guide RNA design for research applications. FEBS J. 2016 September; 283(17:3232-8. doi: 10.1111/febs.13777. Epub 2016 Jun. 22. PMID: 27276584; PMCID: PMC5014588; (2) Hoberecht L, Perampalam P, Lun A, Fortin J P. A comprehensive Bioconductor ecosystem for the design of CRISPR guide RNAs across nucleases and technologies. Nat Commun. 2022 Nov. 2; 13(1):6568. doi: 10.1038/s41467-022-34320-7. PMID: 36323688: PMCID: PMC9630310; (3) Cram D, Kulkarni M1, Buchwaldr M, Rajagopalan N, Bhowmik P, Rozwadowski K, Parkin IAP, Sharpe A G, Kagale S. WheatCRISPR: a web-based guide RNA design tool for CRISPR/Cas9-mediated genome editing in wheat, BMC Plant Biol. 2019 Nov. 6; 19(1):474. doi: 10.1186/s12870-019-2097-z. PMID: 31694550; PMCID: PMC6836449; (4) Pliatsika V, Rigoutsos I. “Off-Spotter”: very fast and exhaustive enumeration of genomic lookalikes for designing CRISPR/Cas guide RNAs. Biol Direct. 2015 Jan. 29; 10:4. doi: 10.1 186/s13062-015-0035-z. PMID: 25630343; PMCID: PMC4326336; (5) Hoof J B. Nødvig C S, Mortensen U H. Genome Editing: CRISPR-Cas9. Methods Mol Biol. 2018; 1775:119-132. doi: 10.1007/978-1-4939-7804-5_11. PMID: 29876814; (6) Labun K, Krause M, Torres Cleuren Y, Valen E. CRISPR Genome Editing Made Easy Through the CHOPCHOP Website. Curr Protoc. 2021 April; 1(4):e46. doi: 10.1002/cpz1.46. PMID: 33905612; (7) Lee C M, Davis T H, Bao G. Examination of CRISPR/Cas9 design tools and the effect of target site accessibility on Cas9 activity. Exp Physiol. 2018 Apr. 1; 103(4):456-460. doi: 10.1113/EP086043. Epub 2017 Apr. 12. PMID: 28303677; PMCID: PMC7266697; (8) Ma S, Lv J, Feng Z, Rong Z, Lin Y. Get ready for the CRISPR/Cas system: A beginner's guide to the engineering and design of guide RNAs. J Gene Med. 2021 November; 23(11):e3377. doi: 10.1002/jgm.3377. Epub 2021 Jul. 28. PMID: 34270141; (9) Hiranniramol K, Chen Y. Wang X. CRISPR/Cas9 Guide RNA Design Rules for Predicting Activity. Methods Mol Bio. 2020:2115:351-364. doi: 10.1007/978-1-0716-0290-4_19. PMID: 32006410; (10) Wiles M V, Qin W V, Cheng A W, Wang 11. CRISPR-Cas9-mediated genome editing and guide RNA design. Mamm Genome. 2015 October; 26(9-10):501-10. doi: 10.1007/s00335-015-9565-z. Epub 2015 May 20. PMID: 25991564: PMCID: PMC4602062; (11) Creutzburg S C A, Wu W Y, Mohanraju P, Swartjes T, Aikan F, Gorodkin J, Staals R H J, van der Oost J. Good guide, bad guide: spacer sequence-dependent cleavage efficiency of Cas12a. Nucleic Acids Res. 2020 Apr. 6; 48(6):3228-3243. doi: 10.1093/nar/gkz1240. PMID: 319891(8; PMCID: PMC7102956; (12) Heigwer P, Boutros M. Cloud-Based Design of Short Guide RNA (sgRNA) Libraries for CRISPR Experiments. Methods Mol Biol. 2021; 2162:3-22. doi: 10.1007/978-1-0716-0687-2_1. PMID: 3292634; (13) Dronina J. Samukaite-Bubniene U. Ramanavicius A. Towards application of CRISPR-Cas12a in th-e design of modern viral DNA detection tools (Review). J Nanobiotechnology. 2022 Jan. 21; 20(1):41. doi: 10.1186/s12951-022-01246-7. PMID: 35062978: PMCID: PMC8777428; (14) Krysler A R, Cromwell C R, Tu T, Jovel J, Hubbard B P. Guide RNAs containing universal bases enable Cas9/Cas12a recognition of polymorphic sequences. Nat Commun. 2022 Mar. 25; 13(1):1617. doi: 10.1038/s41467-022-29202-x. PMID: 35338140; PMCID: PMC8956631; (15) Shin H R, Kweon J, Kim Y. Gene Manipulation Using Fusion Guide RNAs for Cas9 and Cas12a. Methods Mol Biol. 2021; 2162:185-193. doi: 10.1007/978-1-0716-0687-2_10. PMID: 32926383; (16) Schubert M S, Thommandru B, Woodley J, Turk R, Yan S, Kurgan G, McNeill M S, Rettig G R. Optimized design parameters for CRISPR Cas9 and Cas12a homology-directed repair. Sci Rep. 2021 Sep. 30; 11(1):19482. doi: 10.1038/s41598-021-98965-y. PMID: 34593942; PMCID: PMC8484621; (17) Crone M A, MacDonald J T. Freemont P S, Siciliano V. gDesigner: computational design of synthetic gRNAs for Cas12a-based transcriptional repression in mammalian cells. NPJ Syst. Biol Appl 2022 Sep. 16; 8(1):34. doi: 10.1038/s41540-022-00241-w. PMID: 36114193; PMCID: PMC9481559; (18) Konstantakos V, Nentidis A, Krithara A, Paliouras G. CRISPR-Cas9 gRNA efficiency prediction: an overview of predictive tools and the role of deep learning. Nucleic Acids Res. 2022 Apr. 22; 50(7):3616-3637. doi: 10.1093/nar/gkac192. PMID: 35349718; PMC1D: PMC9023298; (19) Wang J, Zhang X, Cheng L, Luo Y. An overview and metanalysis of machine and deep learning-based CRISPR gRNA design tools. RNA Biol. 2020 January; 17(1):13-22. doi: 10.1080/15476286.2019.1669406. Epub 2019 Sep. 27. PMID: 31533522; PMCID: PMC6948960; and (20) Cram D. Kulkarni M, Buchwaldt M, Rajagopalan N. Bhowmik F, Rozwadowski K, Parkin I A P, Sharpe A G, Kagale S. WheatCRISPR: a web-based guide RNA design tool for CRISPR/Cas9-mediated genome editing in wheat. BMC Plant Biol. 2019 Nov. 6; 19(1):474. doi: 10.1186/s12870-019-2097-z. PMID: 31694550; PMCID: PMC6836449; each of which arc incorporated herein by reference in their entireties.

In the specific case of prime editing, in particular, further reference may be made to the following references providing information and tools for the design, synthesis, modification, and structural configuration of pegRNAs: (1) Hsu J Y, Grünewald J, Szalay R, Shih J, Anzalone A V, Lam K C, Shen M W, Petri K, Liu D R, Joung J K, Pinello L. PrimeDesign software for rapid and simplified design of prime editing guide RNAs. Nat Commun. 2021 Feb. 15; 12 1):1034. doi: 10.1038/s41467-021-21337-7. PMID: 33589617; PMCID: PMC7884779; (2) Li Y, Chen J, Tsai S Q, Cheng Y. Easy-Prime: a machine learning-based prime editor design tool, Genome Biol. 2021 Aug. 19; 22(1):235. doi: 10,1186/s 13059-021-02458-0. PMID: 34412673; PMCID: PMC8377858; (3) Zhang W, Petri K, Ma J, Lee H, Tsai C L, Joung J K, Yeh J J. Enhancing CRISPR prime editing by reducing misfolded pegRNA interactions. bioRxiv [Preprint]. 2023 Aug. 15:2023.08.14.553324. doi: 10.1101/2023.08.14.553324. PMID: 37645936; PMCID: PMC10462064; (4) Jin S, Lin Q. Gao Q, Gao C. Optimized prime editing in monocot plants using PlantPegDesigner and engineered plant prime editors (ePPEs). Nat Protoc. 2023 March; 18(3):831-853. doi: 10.1038/s41596-022-00773-9. Epub 2022 Nov. 25. PMID: 36434096; (5) Lin Q. din S, Zong Y, Yu H. Zhu Z. Liu G, Kou L. Wang Y. Qiu J L, Li J, Gao C. High-efficiency prime editing with optimized, paired pegRNAs in plants. Nat Biotechnol. 2021 August; 39(8):923-927. doi: 10.1038/s41587-021-00868-w. Epub 2021 Mar. 25. PMID: 33767395; (6) Standage-Beier K, Tekel S J, Brafman D A, Wang X. Prime Editing Guide RNA Design Automation Using PINE-CONE. ACS Synth Biol. 2021 Feb. 19; 10(2):422-427. doi: 10.1021/acssynbio.0c00445. Epub 2021 Jan. 19. PMID: 33464043; PMCID: PMC7901017; (7) Zhang W, Petri K, Ma J, Lee H, Tsai C L, Joung J K, Yeh J J. Enhancing CRISPR prime editing by reducing misfolded pegRNA interactions. bioRxiv [Preprint]. 2023 Aug. 15:2023.08.14.553324. doi: 10.1101/2023.08.14553324. PMID: 37645936; PMCID: PMC10462064; (8) Chow R D, Chen J S, Shen J, Chen S. A web tool for the design of prime-editing guide RNAs. Nat Biomed Eng. 2021 February; 5(2):190-194. doi: 10.1038/s41551-020-00622-8. Epub 2020 Sep. 28. PMID: 32989284; PMCID: PMC7882013; each of which are incorporated herein by reference in their entireties.

Reference may also be made to the following commercial vendors which synthesize pegRNAs for prime editing applications and provide various tools and instruction for the ordering, design, synthesis, modification, and structural configuration of pegRNAs: GENSCRIPT, SYNTHEGO, TAKARA BIO, INTEGRATED DNA TECHNOLOGIES, LC SCIENCES, HORIZON DISCOVERY; SIGMA-ALDRICH; ORIGENE, and TWIST BIOSCIENCES, among others.

In addition, pegRNAs may be modified with chemical modifications and/or structural modifications for enhancing various properties thereof, including specificity, stability, and limiting off-target activity. One of ordinary skill in the art will be able to modify a guide RNA with any known modification without undue experimentation. Guide modifications are discussed in the following references: (1) Ke Y, Ghalandari B, Huang S, Li S, Huang C, Zhi X, Cui D, Ding X. 2-O-Methyl modified guide RNA promotes the single nucleotide polymorphism (SNP) discrimination ability of CRISPR-Cas12a systems. Chem Sci. 2022 Feb. 1; 13(7):2050-2061. doi: 10.1039/d1sc06832f. PMID: 35308857; PMCID: PMC8848812; (2) Allen D, Rosenberg M, Hendel A. Using Synthetically Engineered Guide RNAs to Enhance CRISPR Genome Editing Systems in Mammalian Cells. Front Genome Ed. 2021 Jan. 28; 2:617910. doi: 10.3389/fgeed.2020,617910. PMID: 34713240; PMCID: PMC8525374; (3) Basila M, Kelley M L, Smith A V B. Minimal 2′-O-methyl phosphorothioate linkage modification pattern of synthetic guide RNAs for increased stability and efficient CRISPR-Cas9 gene editing avoiding cellular toxicity. PLoS One. 2017 Nov. 27; 12(11):e0188593. doi: 10.1371/journial.pone.0188593. PMID: 29176845; PMCID: PMC5703482; (4) Sakovina L, Vokhtantsev I, Vorobyeva M, Vorobyev P, Novopashina D. Improving Stability and Specificity of CRISPR/Cas9 System by Selective Modification of Guide RNAs with 2′-fluoro and Locked Nucleic Acid Nucleotides. Int J Mol Sci. 2022 Nov. 3; 23(2 l): 13460. doi: 10.3390/ijms232113460. PMID: 36362256; PMCID: PMC9655745; (5) Shapiro J, Tovin A, Iancu 0, Allen D, Hendel A. Chemical Modification of Guide RNAs for improved CRISPR Activity in CD34+ Human Hematopoietic Stem and Progenitor Cells. Methods Mol Biol. 2021:2162:37-48. doi: 10.1007/978-1-0716-0687-23. PMID: 32926376; (6) Filippova J, Matveeva A. Zhuravlev E, Stepanov G. Guide RNA modification as a way to improve CRISPR/Cas9-based genome-editing systems. Biochimie. 2019 December; 167:49-60. doi: 10.1016/j.biochi.2019.09.003. Epub 2019 Sep. 4. PMID: 31493470; (7) Hendel A, Bak R O, Clark J T, Kennedy A B, Ryan D E, Roy S, Steinfeld I, Lunstad B D, Kaiser R J, Wilkens A B, Bacchetta R, Tsalenko A, Dellinger D, Bruhn L, Porteus M H. Chemically modified guide RNAs enhance CRISPR-Cas genome editing in human primary cells. Nat Biotechnol. 2015 September; 33(9):985-989. doi: 10.1038/nbt.3290. Epub 2015 Jun. 29. PMID: 26121415; PMCID: PMC4729442; (8) Ryan D E, Taussig D, Steinfeld I, Phadnis S M, Lunstad B D, Singh M, Vuong X, Okochi K D, McCaffrey R, Olesiak M I, Roy S, Yung C W, Curry B, Sampson J R, Bruhn L, Dellinger D J. Improving CRISPR-Cas specificity with chemical modifications in single-guide RNAs. Nucleic Acids Res. 2018 Jan. 25; 46(2):792-803. doi: 10.1093/nar/gkx1199. Erratum in: Nucleic Acids Res. 2022 Mar. 21; 50(5):2986. PMID: 29216382; PMCID: PMC5778453; (9) Palumbo C M, Gutierrez-Bujari J M, O'Geen H, Segal D J, Beal P A. Versatile 3′ Functionalization of CRISPR Single Guide RNA. Chembiochem. 2020 Jun. 2; 21(11):1633-1640. doi: 10.1002/cbic.201900736. Epub 2020 Mar. 5. PMID: 31943634; PMCID: PMC7323579; (10) Mullally G, van Aelst K, Naqvi M M, Diffin F M, Karvelis T, Gasiunas G, Siksnys V, Szczelkun M D. 5′ modifications to CRISPR-Cas9 gRNA can change the dynamics and size of R-loops and inhibit DNA cleavage. Nucleic Acids Res. 2020 Jul. 9:48(12):6811-6823. doi: 10.1093/nar/gkaa477. PMID: 32496535; PMCID: PMC7337959; (12) Lu S, Zhang Y, Yin H. Chimeric DNA-RNA Guide RNA Designs. Methods Mol Biol. 2021; 2162:79-85. doi: 10.1007/978-1-0716-0687-2_6. PMID: 32926379; each of which are incorporated by reference herein in their entireties.

In the specific case of prime editing, pegRNAs may be further modified with chemical modifications and/or structural modifications for enhancing various properties thereof, including specificity, stability, and limiting off-target activity. One of ordinary skill in the art will be able to modify a pegRNA for prime editing with any known modification without undue experimentation. pegRNA modifications are discussed in the following references: (1) Nelson J W, Randolph P B, Shen S P, Everette K A, Chen P J, Anzalone A V, An M, Newby G A, Chen J C, Hsu A, Liu D R. Engineered pegRNAs improve prime editing efficiency. Nat Biotechnol. 2022 March; 40(3):402-410. doi: 10.1038/s41587-021-01039-7. Epub 2021 Oct. 4. Erratum in: Nat Biotechnol. 2021 Dec. 8; PMID: 34608327; PMCID: PMC8930418; (2) Lui B, Dong X, Cheng H, Zheng C, Chen Z, Rodriguez T C, Liang S Q, Xue W, Sontheimer E J. A split prime editor with untethered reverse transcriptase and circular RNA template. Nat Biotechnol 2022 September; 40(9):1388-1393. doi: 10.1038/s41587-022-01255-9. Epub 2022 Apr. 4. PMID: 35379962: each of which are incorporated by reference herein in their entireties.

In some embodiments, the prime editing system further comprises a second guide RNA targeting the complementary strand, allowing the Cas9 nickase to also nick the non-edited strand (called “PE3”), which biases mismatch DNA repair in favor of the edited sequence. In some embodiments, the second guide RNA is designed to recognize the complementary strand of DNA only after the PE3 edit has occurred (called “PE3b”), which reduces indel formation.

In some embodiments, the prime editing system comprises an uracil glycosylase inhibitor. In some embodiments, the prime editing system comprises a Cas9 protein fused to an uracil glycosylase inhibitor. In some embodiments, the cargo comprises an uracil glycosylase inhibitor or a polynucleotide encoding an uracil glycosylase inhibitor. In some embodiments, the cargo comprises a Cas9 protein fused to an uracil glycosylase inhibitor or a polynucleotide encoding a Cas9 protein fused to an uracil glycosylase inhibitor.

Any of the above prime editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

In embodiments, the reverse transcriptase component of the prime editor can be any reverse transcriptase known in the art, or any variant thereof, such as those described in the above published prime editing application or in the scientific literature, such as in: (1) Gao Z, Ravendran S, Mikkelsen N S, Haldrup J, Cai H, Ding X, Paludan S R, Thomsen M K, Mikkelsen J G, Bak R O. A truncated reverse transcriptase enhances prime editing by split AAV vectors. Mol Ther. 2022 Sep. 7; 30(9):2942-2951, doi: 10.1016/j.ymthe.2022.07.001 Epub 2022 Jul. 8. PMID: 35808824: PMCID: PMC9481986; (2) Lan T, Chen H, Tang C, Wei Y, Liu Y, Zhou J, Zhuang Z, Zhang Q, Chen M, Zhou X, Chi Y, Wang J He Y, Lai L., Zou Q. Mini-P E, a prime editor with compact Cas9 and truncated reverse transcriptase. Mol Ther Nucleic Acids. 2023 Aug. 18; 33:890-897. doi: 10.1016/j.omin.2023.08.018. PMID: 37680986; PMCID: PMC10480570; (3) or available biological sequence databases, all of which are incorporated herein by reference.

In addition, the reverse transcriptase may be a retron reverse transcriptase (retron RT), such as any of those described in: (1) U.S. patent application Ser. No. 18/087,673; (2) International PCT Application No. PCT/US2023/061038; (3) International Application No. PCT/US2023/072872; (4) Mestre et al., Nucleic Acids Research, Volume 48, Issue 22, 16 Dec. 2020, Pages 12632-12647; (5) Mestre et al., UG/Abi: “A Highly Diverse Family of Prokaryotic Reverse Transcriptases Associated With Defense Functions,” doi.org/10.1101/2021.12.02.470933; (6) International Application No. PCT/US2023/016262; (7) International Application No. PCT/US2023/016263; (8) International Application No. PCT/US23/72799; (9) International Application NO. PCT/US2022/079220; (10) International Application No. PCT/US2023/016317; and (10) may particularly be any retron selected from Table A of International Application No. PCT/US2023/072872, or any amino acid sequence having at having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A of International Application No. PCT/US2023/072822. The contents of each of the documents in this paragraph are incorporated herein by reference in their entireties.

### Retron Editors

In still other embodiments, the herein disclosed LNPs may be used to encapsulate and deliver a retron editing system. A retron editing system in various embodiments may comprise (a) a retron reverse transcriptase, or a nucleic acid molecule encoding a retron reverse transcriptase, (b) a retron ncRNA (or a nucleic acid molecule encoding same) comprising a modified msd region to include a sequence that is reverse transcribed to form a single strand template DNA sequence (RT-DNA), (c) a nucleic acid programmable nuclease (e.g., a CRISPR Cas9 or Cas12a), and (d) a guide RNA to target the nuclease to a desired target site.

Retrons are defined by their unique ability to produce an unusual satellite DNA known as msDNA (multicopy single-stranded DNA). DNA encoding retrons includes a reverse trancriptase (RT)-coding gene (ret) and a nucleic acid sequence encoding the non-coding RNA (ncRNA), which contains two contiguous and inverted non-coding sequences referred to as the msr and msd. The ret gene and the non-coding RNA (including the msr and msd) are transcribed as a single RNA transcript, which becomes folded into a specific secondary structure following post-transcriptional processing. Once translated, the RT binds the RNA template downstream from the msd locus, initiating reverse transcription of the RNA towards its 5′ end, assisted by the 2′OH group present in a conserved branching guanosine residue that acts as a primer. Reverse transcription halts before reaching the msr locus, and the resulting DNA, the msDNA, remains covalently attached to the RNA template via a 2′-5′ phosphodiester bond and base-pairing between the 3′ ends of the msDNA and the RNA template. The external regions, at the 5′ and 3′ ends of the msd/msr transcript (a1 and a2, respectively) are complementary and can hybridize, leaving the structures located in the msr and msd regions in internal positions. The msr locus, which is not reverse transcribed, forms one to three short stem-loops of variable size, ranging from 3 to 10 base pairs, whereas the msd locus folds into a single/double long hairpin with a highly variable long stem of 10-50 bp in length that is also present in the final msDNA form.

It has recently been reported that retrons may be utilized as a means to provide donor DNA template for HDR-dependent genome editing (e.g., see Lopez et al., “Precise genome editing across kingdoms of life using retron-derived DNA,” Nature Chemical Biology, Dec. 12 2021 18, pages 199-206 (2022)), however, producing sufficient levels of donor DNA template intracellularly to sufficiently support efficient HDR-dependent editing remains a significant challenge.

Retrons have previously been described in the scientific literature, including in the context of retron editing. For example, retrons have been described in the following references, each of which are incorporated herein by reference:

| Title | Pub. | Journal | Author/s | V | Ps |
|---|---|---|---|---|---|
| Recording gene expression | Jul. 27, 2022 | Nature | Bhattarai- | 608 | 217-225 |
| order in DNA by CRISPR | | | Kline; etal. | | |
| addition of retron barcodes. | | | | | |
| Retrons Display Genome | Jun. 1, 2021 | Genetic | | 41 | 15-15 |
| Editing Strengths Even | | Engineering & | | | |
| CRISPR Might Envy | | Biotechnology | | | |
| | | News | | | |
| Retron reverse transcriptase | Mar. 16, 2022 | Nucleic acids | Palka; et al. | 50 | 3490-3504 |
| termination and phage | | research | | | |
| defense are dependent on host | | | | | |
| RNase H1. | | | | | |
| Retrons: Complementing | Aug. 1, 2020 | The CRISPR | Maxwell | 3 | 226-227 |
| CRISPR in Phage Defense. | | journal | | | |
| Systematic prediction of | Dec. 4, 2020 | Nucleic acids | Rodríguez | 48 | 12632-12647 |
| genes functionally associated | | research | Mestre; et al. | | |
| with bacterial retrons and | | | | | |
| classification of the encoded | | | | | |
| tripartite systems. | | | | | |
| Precise genome editing | Aug. 17, 2021 | Protein & cell | Kong; et al. | 12 | 899-902 |
| without exogenous donor | | | | | |
| DNA via retron editing | | | | | |
| system in human cells. | | | | | |
| Precise genome editing | Dec. 23, 2021 | Nature chemical | Lopez; et al. | 18 | 199-206 |
| across kingdoms of life using | | biology | | | |
| retron-derived DNA. | | | | | |
| Prokaryotic reverse | May 13, 2021 | FEMS | González- | 45 | |
| transcriptases: from | | microbiology | Delgado, et al. | | |
| retroelements to specialized | | reviews | | | |
| defense systems | | | | | |
| A function for retrons. | Nov. 17, 2020 | Nature reviews. | Grant Otto | 19 | 3-3 |
| | | Microbiology | | | |
| Retrons and their applications | Oct. 10, 2019 | Nucleic acids | Simon, et al. | 47 | 11007-11019 |
| in genome engineering. | | research | | | |
| High-efficiency retron- | Jan. 1, 2022 | Synthetic | Jiang, et al. | 7 | |
| mediated single-stranded | | biology | | | |
| DNA production in plants. | | (Oxford, | | | |
| | | England) | | | |
| Bacterial retrons enable | Jan. 24, 2022 | The CRISPR | Zhao B, et al. | 5 | 31-39 |
| precise gene editing in human | | journal | | | |
| cells | | | | | |
| Like CRISPR, mystery gene | Nov. 20, 2020 | Science (New | Pennisi | 370 | 898-899 |
| editor began as a virus | | York, N.Y.) | | | |
| fighter. | | | | | |
| Bacterial Retrons Function In | Nov. 5, 2020 | Cell | Millman, et al. | 183 | 1551-1561 |
| Anti-Phage Defense. | | | | | |
| Multiplex generation, | May 4, 2020 | ACS synthetic | Lim, et al. | 9 | 1003-1009 |
| tracking, and functional | | biology | | | |
| screening of substitution | | | | | |
| mutants using a crispr/retron | | | | | |
| system | | | | | |
| Multiple origins of reverse | Jul. 11, 2019 | RNA biology | Toro, et al. | 16 | 1486-1493 |
| transcriptases linked to | | | | | |
| CRISPR-Cas systems. | | | | | |
| Retroelement-Based Genome | Oct. 26, 2018 | ACS synthetic | Simon, et al. | 7 | 2600-2611 |
| Editing and Evolution. | | biology | | | |

In addition, retrons have previously been described in the patent literature, including in the context of retron editing. For example, retrons have been described in the following references, each of which are incorporated herein by reference:

| Publication No. | TITLE |
|---|---|
| US 2020/0115706 A1 | METHOD OF RECORDING MULTIPLEXED BIOLOGICAL |
| | INFORMATION INTO A CRISPR ARRAY USING A RETRON |
| EP 3510151 A4 | HIGH-THROUGHPUT PRECISION GENOME EDITING |
| US 2019/0330619 A1 | HIGH-THROUGHPUT PRECISION GENOME EDITING |
| EP 3510151 A1 | HIGH-THROUGHPUT PRECISION GENOME EDITING |
| WO 2018/191525 A1 | METHOD OF RECORDING MULTIPLEXED BIOLOGICAL |
| | INFORMATION INTO A CRISPR ARRAY USING A RETRON |
| US 2018/0127759 A1 | DYNAMIC GENOME ENGINEERING |
| WO 2018/049168 A1 | HIGH-THROUGHPUT PRECISION GENOME EDITING |
| US 2017/0204399 A1 | GENOMICALLY-ENCODED MEMORY IN LIVE CELLS |
| EP 3180430 A1 | GENOMICALLY-ENCODED MEMORY IN LIVE CELLS |
| CA 2488328 C | RETRONS FOR GENE TARGETING |
| WO 2016/025719 A1 | GENOMICALLY-ENCODED MEMORY IN LIVE CELLS |
| U.S. Pat. No. 8,932,860 B2 | RETRONS FOR GENE TARGETING |
| EP 1517992 B1 | RETRONS FOR GENE TARGETING |
| AU 2003/233734 C1 | RETRONS FOR GENE TARGETING |
| AU 2003/233734 B2 | RETRONS FOR GENE TARGETING |
| US 2009/0123991 A1 | RETRONS FOR GENE TARGETING |
| US 2005/0250207 A1 | RETRONS FOR GENE TARGETING |
| EP 1517992 A2 | RETRONS FOR GENE TARGETING |
| WO 2003/104470 A3 | RETRONS FOR GENE TARGETING |
| AU 2003/233734 A1 | RETRONS FOR GENE TARGETING |
| CA 2488328 A1 | RETRONS FOR GENE TARGETING |
| WO 2003/104470 A2 | RETRONS FOR GENE TARGETING |

In some embodiments, the LNP-based retron editing system can be used for genome editing a desired site. A retron is engineered with a heterologous nucleic acid sequence encoding a donor polynucleotide (“template or donor nucleotide sequence” or “template DNA”)~suitable for use with nuclease genome editing system. The nuclease is designed to specifically target a location proximal to the desired edit (the nuclease should be designed such that it will not cut the target once the edit is properly installed). The nuclease (e.g., CAS or non-CAS) is linked to the retron, either by direct fusion to the RT or by fusion of the msDNA to the gRNA (only applicable for RNA-guided nucleases). A heterologous nucleic acid sequence is inserted into the retron msd.

In some embodiments, the heterologous nucleic acid sequence has 10-100 or more bp of homologous nucleic acid sequence to the genome on both sides of the desired edit. The desired edit (insertion, deletion, or mutation) is in between the homologous sequence.

In some embodiments, donor polynucleotides comprise a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relate to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit can be integrated into the genomic DNA by HDR at the genomic target locus recognized (i.e., having sufficient complementary for hybridization) by the 5′ and 3′ homology arms.

In some embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In some embodiments, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g. 10 nucleotides or more, 15 nucleotides or more, 20 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another. However, in some instances the 5′ and 3′ homology arms are not necessarily equal in length to one another. For example, one homology arm may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g. one may be 40% shorter or more, 50% shorter or more, sometimes 60% shorter or more, 70% shorter or more, 80% shorter or more, 90% shorter or more, or 95% shorter or more than the other homology arm.

The donor polynucleotide may be used in combination with an RNA-guided nuclease, which is targeted to a particular genomic sequence (i.e., genomic target sequence to be modified) by a guide RNA. A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to the sequence of a minor allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted minor allele may be a common genetic variant or a rare genetic variant. In some embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to remove the mutation from a gene. Alternatively, the gRNA can be designed with a sequence complementary to the sequence of a major or wild-type allele to target the nuclease-gRNA complex to the allele for the purpose of genome editing to introduces a mutation into a gene in the genomic DNA of the cell, such as an insertion, deletion, or substitution. Such genetically modified cells can be used, for example, to alter phenotype, confer new properties, or produce disease models for drug screening.

In some embodiments, the RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system Class 1, Type I, II, or III Cas nucleases; Class 2, Type II nuclease (such as Cas9); a Class 2, Type V nuclease (such as Cpf1), or a Class 2, Type VI nuclease (such as C2c2). Examples of Cas proteins include Casl, CaslB, Cas2, Cas3, Cas4, Cas5, Cas5e (CasD), Cas6, Cas6e, Cas6f, Cas7, Cas8al, Cas8a2, Cas8b, Cas8c, Cas9 (Csnl or Csxl2), CaslO, CaslOd, CasF, CasG, CasH, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, and Cul966, and homologs or modified versions thereof.

In some embodiments, a Class 1, type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced.

Cas9 sequences from a number of bacterial species are well known in the art and listed in the National Center for Biotechnology Information (NCBI) database. See, for example, NCBI entries for Cas9 from: Streptococcus pyogenes (WP 002989955, WP_038434062, WP_011528583); Campylobacter jejuni (WP_022552435, YP 002344900), Campylobacter coli (WP 060786116); Campylobacter fetus (WP 059434633); Corynebacterium ulcerans (NC_015683, NC_017317); Corynebacterium diphtheria (NC_016782, NC_016786); Enterococcus faecalis (WP 033919308); Spiroplasma syrphidicola (NC 021284); Prevotella intermedia (NC 017861); Spiroplasma taiwanense (NC 021846); Streptococcus iniae (NC 021314); Belliella baltica (NC 018010); Psychroflexus torquisl (NC O 18721); Streptococcus thermophilus (YP 820832), Streptococcus mutans (WP 061046374, WP 024786433); Listeria innocua (NP 472073); Listeria monocytogenes (WP 061665472); Legionella pneumophila (WP 062726656); Staphylococcus aureus (WP_001573634); Francisella tularensis (WP_032729892, WP_014548420), Enterococcus faecalis (WP 033919308); Lactobacillus rhamnosus (WP 048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacterid. 198(5): 797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The genomic target site will typically comprise a nucleotide sequence that is complementary to the gRNA and may further comprise a protospacer adjacent motif (PAM). In some embodiments, the target site comprises 20-30 base pairs in addition to a 3 or more base pair PAM. Typically, the first nucleotide of a PAM can be any nucleotide, while the two or more other nucleotides will depend on the specific Cas9 protein that is chosen. Exemplary PAM sequences are known to those of skill in the art and include, without limitation, NNG, NGN, NAG, and NGG, wherein N represents any nucleotide. In some embodiments, the allele targeted by a gRNA comprises a mutation that creates a PAM within the allele, wherein the PAM promotes binding of the Cas9-gRNA complex to the allele.

In some embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, or Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

C2c1 (Cas12b) is another class II CRISPR/Cas system RNA-guided nuclease that may be used. C2c1, similarly to Cas9, depends on both a crRNA and tracrRNA for guidance to target sites.

See, e.g., Shmakov et al. (2015) Mol Cell. 60(3):385-397, Zhang et al. (2017) Front Plant Sci. 8:177; herein incorporated by reference.

In yet another embodiment, an engineered RNA-guided Fokl nuclease may be used. RNA-guided Fokl nucleases comprise fusions of inactive Cas9 (dCas9) and the Fokl endonuclease (FokI-dCas9), wherein the dCas9 portion confers guide RNA-dependent targeting on Fokl. For a description of engineered RNA-guided Fold nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

In other embodiments, any other Cas enzymes and variants described in other sections of the application (all incorporated herein) can be used similarly.

In some embodiments, the RNA-guided nuclease is provided in the form of a protein, optionally where the nuclease is complexed with a gRNA to form a ribonucleoprotein (RNP) complex. In some embodiments, the RNA-guided nuclease is provided by a nucleic acid encoding the RNA-guided nuclease, such as an RNA (e.g., messenger RNA) or DNA (expression vector). In some embodiments, the RNA-guided nuclease and the gRNA are both provided by vectors, such as the vectors and the vector system described in other parts of the application (all incorporated herein by reference). Both can be expressed by a single vector or separately on different vectors. The vectors encoding the RNA-guided nuclease and gRNA may be included in the vector system comprising the engineered retron msr gene, msd gene and ret gene sequences. In some embodiments, the RNA-guided nuclease is fused to the RT and/or the msDNA.

The RNP complex may be administered to a subject or delivered into a cell by methods known in the art, such as those described in U.S. Pat. No. 11,390,884, which is incorporated by reference herein in its entirety. In some embodiments, the endonuclease/gRNA ribonucleoprotein (RNP) complexes are delivered to cells by electroporation. Direct delivery of the RNP complex to a subject or cell eliminates the need for expression from nucleic acids (e.g., transfection of plasmids encoding Cas9 and gRNA). It also eliminates unwanted integration of DNA segments derived from nucleic acid delivery (e.g., transfection of plasmids encoding Cas9 and gRNA). An endonuclease/gRNA ribonucleoprotein (RNP) complex usually is formed prior to administration.

Codon usage may be optimized to further improve production of an RNA-guided nuclease and/or reverse transcriptase (RT) in a particular cell or organism. For example, a nucleic acid encoding an RNA-guided nuclease or reverse transcriptase can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease or reverse transcriptase is introduced into cells, the protein can be transiently, conditionally, or constitutively expressed in the cell.

In some embodiments, the engineered retron used for genome editing with nuclease genome editing systems can further include accessory or enhancer proteins for recombination. Examples of recombination enhancers can include nonhomologous end joining (NHEJ) inhibitors (e.g., inhibitor of DNA ligase IV, a KU inhibitor (e.g., KU70 or KU80), a DNA-PKc inhibitor, or an artemis inhibitor) and homologous directed repair (HDR) promoters, or both, that can enhance or improve more precise genome editing and/or the efficiency of homologous recombination. In some embodiments, the recombination accessory or enhancers can comprise C-terminal binding protein interacting protein (CtIP), cyclinB2, Rad family members (e.g. Rad50, Rad51, Rad52, etc).

CtIP is a transcription factor containing C₂H2 zinc fingers that are involved in early steps of homologous recombination. Mammalian CtIP and its orthologs in other eukaryotes promote the resection of DNA double-strand breaks and are essential for meiotic recombination. HDR may be enhanced by using Cas9 nuclease associated (e.g. fused) to an N-terminal domain of CtIP, an approach that forces CtIP to the cleavage site and increases transgene integration by HDR. In some embodiments, an N-terminal fragment of CtIP, called HE for HDR enhancer, may be sufficient for HDR stimulation and requires the CtIP multimerization domain and CDK phosphorylation sites to be active. HDR stimulation by the Cas9-HE fusion depends on the guide RNA used, and therefore the guide RNA will be designed accordingly.

Using the gene editing system described herein, any target gene or sequence in a host cell can be edited or modified for a desired trait, including but not limited to: Myostatin (e.g., GDF8) to increase muscle growth; Pc POLLED to induce hairlessness; KISS1R to induce bore taint; Dead end protein (dnd) to induce sterility; Nano2 and DDX to induce sterility; CD163 to induce PRRSV resistance; RELA to induce ASFV resilience; CD18 to induce Mannheimia (Pasteurella) haemolytica resilience; NRAMPl to induce tuberculosis resilience; Negative regulators of muscle mass (e.g., Myostatin) to increase muscle mass.

Any of the above retron editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### TnpB Editors

In other embodiments, the herein disclosed LNPs may be used to encapsulate and deliver a TnpB editing system and/or components thereof. A TnpB editing system in various embodiments may comprise (a) a TnpB protein, or a nucleic acid molecule encoding a TnpB protein, (b) a TnpB guide RNA known as an “reRNA” or “right end RNA”, and optionally one or more additional components, including (c) an effector domain or otherwise accessory protein, and (d) a DNA template (e.g., a DNA donor for HDR-dependent repair at the TnpB-cut target site.

In various embodiments, the TnpB protein can be naturally occurring or the TnpB can be an engineered variant thereof and can be used in various applications, including precision gene editing in cells, tissues, organs, or organisms. The TnpB-based gene editing systems comprise a TnpB polypeptide and a nucleic acid component capable of forming a complex with the TnpB polypeptide which directs the complex to a target nucleotide sequence (e.g., a genomic target sequence such as a disease-associated gene). The TnpB gene editing systems contemplated herein may also be modified with one or more additional effector or accessory functions, such as a nuclease, recombinase, ligase, reverse transcriptase, polymerase, deaminase, etc. to provide additional genome editing functionality. In addition, the TnpB gene editing systems contemplated herein can utilize a nuclease-limited or nuclease-deficiently TnpB variant. Normal TnpB nuclease activity cuts both strands of a target DNA, however, TnpB nickases (having only the ability to cut one of the two strands but not both strands) and nuclease-inactive or “dead” TnpB (which does not cut either strand) may also be used into the TnpB systems described herein, particularly when combined with at least another genome editing functionality, such as a deaminase (for base editing functionality) or a reverse transcriptase (for prime editing functionality). Thus, disclosed herein are TnpB systems that may function as nuclease, nickases, or catalytically inactive polynucleotide binding proteins that can be coupled with other functional domains, such as deaminases, recombinase, ligases, polymerases (e.g., reverse transcriptase), nucleases, or reverse transcriptases.

In one embodiment, the TnpB systems and related compositions may specifically target single-strand or double-strand DNA. In one embodiment, the TnpB system may bind and cleave double-strand DNA. In one embodiment, the TnpB system may bind to double-stranded DNA without introducing a break to either of the strands. In one embodiment, the TnpB polypeptides or nuclease/nucleic acid component complexes may open, disrupting the continuity of one of the two DNA strands, thereby introducing a nick of the double stranded DNA. In an embodiment, and without being bound by theory, the size and configuration of the TnpB systems allows exposure to the non-targeting strand, which may be in single-stranded form, to allow for for the ability to modify, edit, delete or insert polynucleotides on the non-target strand. In an embodiment, this accessibility further allows for enhanced editing outcomes on the target and/or non-target strand, e.g., increased specificity, enhanced editing efficiency.

In one aspect, embodiments disclosed herein are directed to compositions comprising a TnpB and a reRNA capable of forming a complex with the TnpB and directing site-specific binding of the TnpB to a target sequence on a target polynucleotide.

### TnpB Polypeptides

Any TnpB polypeptide may be utilized with the compositions described herein. The below description of various TnpBs which can be used in connection with the presently disclose TnpB editing systems is not meant to be limiting in any way. The TnpB editing systems disclosed herein may comprise a canonical or naturally-occurring TnpB, or any ortholog TnpB protein, or any variant TnpB protein—including any naturally occurring variant, mutant, or otherwise engineered version of TnpB—that is known or which can be made or evolved through a directed evolutionary or otherwise mutagenic process. In various embodiments, the TnpB or TnpB variants can have a nickase activity, i.e., only cleave of strand of the target DNA sequence. In other embodiments, the TnpB or TnpB variants have inactive nucleases, i.e., are “dead” TnpB proteins. Other variant TnpB proteins that may be used are those having a smaller molecular weight than the canonical TnpB (e.g., for easier delivery) or having modified amino acid sequences or substitutions.

Examples of TnpB proteins are provided as follows; however, these specific examples are not meant to be limiting. The TnpB editing systems of the present disclosure may use any suitable TnpB protein.

In various embodiments, the TnpB editing systems of the present disclosure may include one or more TnpB polypeptides selected from those disclosed in WO 2023/240261A1, or a polypeptide having at least 70%, 75%, 80%, 85%, 90%, 95%, 99%, or 100% sequence identity with one or more of the TnpB polypeptides of WO 2023/240261A1, which is incorporated by reference herein, in its entirety.

In various other embodiments, the TnpB editing systems of the present disclosure may include one or more TnpB polypeptides and reRNAs disclosed in any of the following published applications, or a polypeptide (or reRNA as the case may be) having at least 70%, 75%, 80%, 85%, 90%, 95%, 99%, or 100% sequence identity with one or more of the TnpB polypeptides or reRNAs disclosed therein: US2023/0056577; US2023/0051396 A1; U.S. Ser. No. 11/578,313 B2; US2023/0040216 A1; WO2023/015259 A2; US2023/0032369 A1; US2023/0033866 A1; WO2023/004430 A1; U.S. Ser. No. 11/560,555 B2; WO2023/275601 A1; WO2022/253903 A1; WO2022/248607 A2; US2022/0372525 A1; US2022/0348929 A1; US2022/0348925 A1; U.S. Ser. No. 11/453,866 B2; WO2022/173830 A1; WO2022/174144 A1; WO2022/159892 A1; WO2022/150651 A1; U.S. Ser. No. 11/384,344 B2; WO2022/140572 A1; US2022/0195503 A1; WO2022/098923 A1; WO2022/087494 A1; WO2022/086846 A2; WO2022/076425 A1; WO2022/076890 A1; WO2021/257997 A2; WO2021/247924 A1; US2021/0380956 A1; U.S. Ser. No. 11/180,751 B2; WO2021/188729 A1; WO2021/188286 A2; WO2021/183807 A1; WO2021/159020 A2; US2021/0214697 A1; US2021/0166783 A1; WO2021/050601 A1; EP3009511 B2; US2020/0291395 A1; US2020/0239896 A1; WO2019/178428 A1; US2012/0178668 A1; U.S. Pat. No. 7,608,450 B2; US2004/0091856 A1; US2004/0009477 A1; US2003/0134302 A1; U.S. Pat. No. 6,562,958 B1; and WO1999/051766 A1, each of which are incorporated in their entireties by reference.

In certain example embodiments, the TnpB polypeptides are between 175 and 800 amino acids in size, between 200 and 790 amino acids in size, between 200 and 780 amino acids in size, between 200 and 770 amino acids in size, between 200 and 760 amino acids in size, between 200 and 750 amino acids in size, between 200 and 740 amino acids in size, between 200 and 730 amino acids in size, between 200 and 720 amino acids in size, between 200 and 720 amino acids in size, between 200 and 710 amino acids in size, between 200 and 700 amino acids in size, between 200 and 690 amino acids in size, between 200 and 680 amino acids in size, between 200 and 670 amino acids in size, between 200 and 660 amino acids in size, between 200 and 650 amino acids in size, between 200 and 640 amino acids in size, between 200 and 630 amino acids in size, between 200 and 620 amino acids in size, between 200 and 610 amino acids in size, between 200 and 600 amino acids in size, between 200 and 590 amino acids in size, between 200 and 580 amino acids in size, between 200 and 570 amino acids in size, between 200 and 560 amino acid, between 200 between 550 amino acids, between 200 and 540 amino acids, between 200 and 530 amino acids, between 200 and 520 amino acids, between 200 and 510 amino acids, between 200 and 500 amino acids, between 200 and 490 amino acids, between 200 and 480 amino acids, between 200 and 470 amino acids, between 200 and 460 amino acids, between 200 and 450 amino acids, between 200 and 440 amino acids, between 200 and 430 amino acids, between 200 and 420 amino acids, between 200 and 410 amino acids, between 210 and 500 amino acids, between 220 and 500 amino acids. Between 230 and 500 amino acids, between 240 and 500 amino acids, between 250 and 500 amino acids, between 260 and 500 amino acids, between 270 and 500 amino acids, between 280 and 500 amino acids, between 290 and 500 amino acids, between 300 and 500 amino acids, between 250 and 490 amino acids, between 250 and 480 amino acids, between 250 and 490 amino acids, or between 250 and 600 amino acids. In one embodiment, the TnpB polypeptide is between 300 and 500 amino acids, or between 350 and 450 amino acids.

In one embodiment, the TnpB polypeptides may comprise a modified naturally occurring protein, functional fragment or truncated version thereof, or a non-naturally occurring protein. In one embodiment, the TnpB polypeptide comprises one or more domains originating from other TnpB polypeptides, more particularly originating from different organisms. In one embodiment, the TnpB polypeptides may be designed by in silico approaches. Examples of in silico protein design have been described in the art and are therefore known to a skilled person.

The TnpB polypeptides also encompass homologs or orthologs of TnpB polypeptides whose sequences are specifically described herein (such as the sequences of Table A). The terms “ortholog” and “homolog” are well known in the art. By means of further guidance, a “homolog” of a protein as used herein is a protein of the same species which performs the same or a similar function as the protein it is a homolog of. Homologous proteins may but need not be structurally related, or are only partially structurally related. An “ortholog” of a protein as used herein is a protein of a different species which performs the same or a similar function as the protein it is an orthologue of.

Orthologous proteins may be, but may not always be, structurally related or are only partially structurally related. In particular embodiments, the homolog or ortholog of a TnpB polypeptide such as referred to herein has a sequence homology or identity of at least 80%, at least 81%, at least 82%, at least 83%, at least 84% at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with a TnpB polypeptide, more specifically with a TnpB sequence identified in Table A. In particular embodiments, a homolog or ortholog is identified according to its domain structure and/or function. Sequence alignments conducted as described herein, as well as folding studies and domain predictions can aid in the identification of a homolog or ortholog with the structural and functional characteristics identifying TnpB polypeptides, particularly those with conserved residues, including catalytic residues, and domains of TnpB polypeptides.

In one embodiment, the TnpB polypeptide comprises at least at least one RuvC-like nuclease domain. The RuvC domain may comprise conserved catalytic amino acids indicative of the RuvC catalytic residue. In an example embodiment, the RuvC catalytic residue may be referenced relative to D191, E278, and D361 of the TnpB of D. radiodurans or a corresponding amino acid in an aligned sequence. In an aspect, the RuvC domain may comprise multiple subdomains, e.g., RuvC-I, RuvC-II and RuvC-III. The subdomains may be separated by intervening amino acid sequence of the protein.

In one embodiment, examples of the RuvC domain include any polypeptides a structural similarity and/or sequence similarity to a RuvC domain described in the art. In some examples, the RuvC domain may have an amino acid sequence that share at least 50%, at least 55%, at least 60%, at least 65%, at least 70%, at least 75%, at least 80%, at least 85%, at least 90%, at least 95%, at least 99%, or 100% sequence identity with RuvC domains known in the art. One of ordinary skill in the art can modify, substitute, or otherwise alter the activity of the RuvC domain to alter the nuclease activity, such as whether and/or where the nuclease cuts the DNA.

In embodiments, the TnpB polypeptide has a nuclease activity. In one embodiment, the TnpB and the targeting RNA (e.g., the reRNA) can direct sequence-specific nuclease activity. The cleavage may result in a 5′ overhang. The cleavage may occur distal to a target-adjacent motif (TAM), and may occur at the site of the spacer (i.e., the spacer of the reRNA which is complementary to the target sequences) annealing site or 3′ of the target sequence. In an aspect, the TnpB cleaves at multiple positions within and beyond the nucleic acid component annealing site. In an aspect, DNA cleavage occurs 15, 16, 17, 18, 19, 20, 21, 22, 23, 24, 25 or more base pairs distal to the TAM and results in a 5′ overhang. In various embodiments, the TnpB has a nuclease activity against single-stranded DNA. In other embodiments, the TnpB has a nuclease activity against double-stranded DNA.

### TnpB Modifications

In various aspects, the present disclosure provides one or more modifications of TnpB comprising TnpB fusions, TnpB mutations to increase sufficiency and/or efficiency and modification of TnpB reRNA. In some embodiments, one or more domains of the TnpB are modified, e.g., wedge domain, corresponding to the β-barrel, REC—helical bundle, RuvC—RuvC domain with the inserted helical hairpin (HH) and the zinc-finger domain (ZnF).

Without intending to be limited to any particular theory, TnpB operates as a homodimer with one DNA molecule and for some orthologs, its ability to form this conformation may be efficacy limiting. Takeda, Satoru N et al. “Structure of the miniature type V-F CRISPR-Cas effector enzyme.” Molecular cell vol. 81, 3 (2021): 558-570.e3.

Karvelis et al. demonstrated Deinococcus radiodurans ISDra2 TnpB to be an RNA-directed nuclease guided by RE-derived RNA (reRNA) to cleave DNA next to the 5′ TTGAT transposon associated motif (TAM). Karvelis, T., Druteika, G., Bigelyte, G. et al. Transposon-associated TnpB is a programmable RNA-guided DNA endonuclease. Nature 599, 692-696 (2021).

Without being bound by theory, it is contemplated that TnpB likely operates as a homodimer. Recent studies show that Cas9-Cas9 fusions displayed higher levels of genome modification and a higher proportion of these editing events were precise deletions than are observed for two independent Cas9 nucleases. Bolukbasi, M. F., Liu, P., Luk, K. et al. Orthogonal Cas9-Cas9 chimeras provide a versatile platform for genome editing. Nat Commun 9, 4856 (2018).

Accordingly, in one embodiment, a TnpB is fused to a second TnpB or the like, for example TnpB-TnpB or TnpB-Cas9. Such dual-nuclease formats comprise one TnpB component displaying expanded targeting and/or enhanced specificity and the second TnpB component having nuclease activity. In other preferred embodiments, a TnpB is fused to two or more nuclease proteins.

The TnpB polypeptide may comprise one or more modifications. As used herein, the term “modified” with regard to a TnpB polypeptide generally refers to a TnpB polypeptide having one or more modifications or mutations (including point mutations, truncations, insertions, deletions, chimeras, fusion proteins, etc.) compared to the wild type counterpart from which it is derived (e.g., from a TnpB sequence from Tables B or C). By derived is meant that the derived enzyme is largely based, in the sense of having a high degree of sequence or structural homology with, a wildtype enzyme, but that it has been mutated (modified) in some way as known in the art or as described herein.

The modified proteins, e.g., modified TnpB polypeptide may be catalytically inactive (dead). As used herein, a catalytically inactive or dead nuclease may have reduced, or no nuclease activity compared to a wildtype counterpart nuclease. In some cases, a catalytically inactive or dead nuclease may have nickase activity. In some cases, a catalytically inactive or dead nuclease may not have nickase activity. Such a catalytically inactive or dead nuclease may not make either double-strand or single-strand break on a target polynucleotide but may still bind or otherwise form complex with the target polynucleotide.

In an embodiment, eukaryotic homologues of bacterial TnpB may be utilized in the present disclosure. These TnpB-like proteins, Fanzor 1 and Fanzor 2, while having a shared amino acid motif in their C-terminal half regions, are variable in their N terminal regions.

In one embodiment, the modifications of the TnpB polypeptide may or may not cause an altered functionality. By means of example, modifications which do not result in an altered functionality include for instance codon optimization for expression into a particular host, or providing the nuclease with a particular marker (e.g. for visualization). Modifications with may result in altered functionality may also include mutations, including point mutations, insertions, deletions, truncations (including split nucleases), etc., as well as chimeric nucleases (e.g., comprising domains from different orthologues or homologues) or fusion proteins. Fusion proteins may without limitation include, for instance, fusions with heterologous domains or functional accessory domains (e.g., localization signals, catalytic domains, etc.). In one embodiment, various different modifications may be combined (e.g., a mutated nuclease which is catalytically inactive and which further is fused to a functional domain, such as for instance to induce DNA methylation or another nucleic acid modification, such as including without limitation, a break (e.g. by a different nuclease (domain)), a mutation, a deletion, an insertion, a replacement, a ligation, a digestion, a break or a recombination).

As used herein, “altered functionality” includes without limitation an altered specificity (e.g., altered target recognition, increased (e.g., “enhanced” TnpB polypeptide) or decreased specificity, or altered TAM recognition), altered activity (e.g., increased or decreased catalytic activity, including catalytically inactive nucleases or nickases), and/or altered stability (e.g., fusions with destabilization domains).

Examples of all these modifications are known in the art. It will be understood that a “modified” nuclease as referred to herein, and in particular a “modified” TnpB polypeptide or system or complex preferably still has the capacity to interact with or bind to the polynucleic acid (e.g., in complex with the nucleic acid component molecule). Such modified TnpB polypeptide can be combined with the deaminase protein or active domain thereof as described herein.

In one embodiment, an unmodified TnpB polypeptides may have cleavage activity. In one embodiment, the TnpB polypeptides may direct cleavage of one or both nucleic acid (DNA or RNA) strands at the location of or near a target sequence, such as within the target sequence and/or within the complement of the target sequence or at sequences associated with the target sequence. In one embodiment, the TnpB polypeptides may direct cleavage of one or both DNA or RNA strands within about 1, 2, 3, 4, 5, 6, 7, 8, 9, 10, 15, 20, 25, 50, 100, 200, 500, or more base pairs or nucleotides from the first or last nucleotide of a target sequence. In one embodiment, the cleavage may be staggered, i.e., generating sticky ends. In one embodiment, the cleavage is a staggered cut with a 5′ overhang. In one embodiment, the cleavage is a staggered cut with a 5′ overhang of I to 5 or up to 10 nucleotides. In particular embodiments, the TnpB polypeptides cleave DNA strands.

In one embodiment, a TnpB polypeptide may be mutated with respect to a corresponding wild-type enzyme (e.g., the TnpB polypeptides of Tables B and C) such that the mutated TnpB lacks the ability to cleave one or both strands of a target polynucleotide containing a target sequence. As a further example, two or more catalytic domains of a TnpB polypeptide (e.g., RuvC) may be mutated to produce a mutated TnpB polypeptide substantially lacking all DNA cleavage activity. In one embodiment, a TnpB polypeptide may be considered to substantially lack all polynucleotide cleavage activity when the polynucleotide cleavage activity of the mutated enzyme is no more than 25%, no more than 10%, no more than 5%, no more than 1%, no more than 0.1%, no more than 0.01% of the nucleic acid cleavage activity of the non-mutated form of the enzyme; an example can be when the nucleic acid cleavage activity of the mutated form is nil or negligible as compared with the non-mutated form.

In one embodiment, the TnpB polypeptide may comprise one or more modifications resulting in enhanced activity and/or specificity, such as including mutating residues that stabilize the targeted or non-targeted strand. In one embodiment, the altered or modified activity of the engineered TnpB polypeptide comprises increased targeting efficiency or decreased off-target binding. In one embodiment, the altered activity of the engineered TnpB polypeptide comprises modified cleavage activity. In one embodiment, the altered activity comprises increased cleavage activity as to the target polynucleotide loci. In one embodiment, the altered activity comprises decreased cleavage activity as to the target polynucleotide loci. In one embodiment, the altered activity comprises decreased cleavage activity as to off-target polynucleotide loci. In one embodiment, the modified nuclease comprises a modification that alters association of the protein with the nucleic acid molecule comprising RNA, or a strand of the target polynucleotide loci, or a strand of off-target polynucleotide loci.

In an aspect of the disclosure, the engineered TnpB polypeptide comprises a modification that alters formation of the TnpB polypeptide and related complex. In one embodiment, the altered activity comprises increased cleavage activity as to off-target polynucleotide loci. Accordingly, in one embodiment, there is increased specificity for target polynucleotide loci as compared to off-target polynucleotide loci. In other embodiments, there is reduced specificity for target polynucleotide loci as compared to off-target polynucleotide loci. In one embodiment, the mutations result in decreased off-target effects (e.g. cleavage or binding properties, activity, or kinetics), such as in case for TnpB polypeptide for instance resulting in a lower tolerance for mismatches between target and the reRNA. Other mutations may lead to increased off-target effects (e.g., cleavage or binding properties, activity, or kinetics). Other mutations may lead to increased or decreased on-target effects (e.g., cleavage or binding properties, activity, or kinetics). In one embodiment, the mutations result in altered (e.g., increased or decreased) activity, association or formation of the functional nuclease complex. Examples mutations include mutation of negative or neutral residues to positively charged residues, or positively charged residues to neutral or neutral residues to negative residues and/or (evolutionary) conserved residues, such as conserved positively charged residues, in order to enhance specificity. In one embodiment, such residues may be mutated to uncharged residues, such as alanine. Because the TnpB polypeptide interacts with guide or bound DNA over the length of the TnpB polypeptide, mutation of residues across the TnpB polypeptide may be utilized for altered activity. In an aspect, the TnpB polypeptide residues for mutation are altered based on amino acid sequence positions of Deinococcus radiodurans ISDra2, see, e.g. Karvelis et al., Nature 599, 692-696 (2021).

Preferably, one or more TnpB comprises one or more mutated residues in the Rec domain and optionally these mutated residues are hydrophobic. Alternatively, one or more TnpB comprises mutated residues in the RuvC domain. Preferably, one or more of the mutated residues typically form a hydrogen bond with another TnpB monomer. More preferably, a combination of the two sets of mutations as described above.

In yet other embodiments, the TnpB-nuclease fusions are linked using a polypeptide comprising glycine and serine residues or unstructured XTEN protein polymer.

In other exemplary embodiments, the TnpB-nuclease fusions are linked using an RNA wherein the RNA comprises a guide RNA or a reRNA.

In further embodiments, the TnpB-nuclease fusions comprise one or more nuclear localization signals selected from but not limited to SV40, c-Myc, NLP-1.

Also described herein are methods and compositions for increasing the TnpB-mediated editing efficiency. In some aspects, the editing efficiency is greater than 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99%.

Additionally described herein are methods and compositions for increasing the TnpB-mediated editing specificity. In some aspects, the editing specificity is greater than 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99%.

### TnpB Accessory Domains/Proteins

In other aspect, the TnpB-based genome perturbation systems may comprise one or more additional accessory proteins having genome modifying functions, including recombinases, invertases, nucleases, polymerases, ligases, deaminases, or reverse transcriptases. In various embodiments, the accessory proteins may be provided separately. In other embodiments, the accessory proteins may be fused to TnpB, optionally with a linker.

Liu et al. has recently developed base editing as a technology that edits target nucleotides without creating DSBs or relying on HDR. Direct modification of DNA bases by Cas-fused deaminase enzymes allows for C·G to T·A, or A·T to G·C, base pair conversions in a short target window (-5-7 bases) with very high efficiency. Komor, A. C., Kim, Y. B., Packer, M. S., Zuris, J. A. & Liu, D. R. Programmable editing of a target base in genomic DNA without double-stranded DNA cleavage. Nature 533, 420-424 (2016). Nishida, K. et al. Targeted nucleotide editing using hybrid prokaryotic and vertebrate adaptive immune systems. Science 353, aaf8729 (2016). 6. Gaudelli, N. M. et al. Programmable base editing of A·T to G·C in genomic DNA without DNA cleavage. Nature 551, 464-471 (2017). Kim, Y. B. et al. Increasing the genome-targeting scope and precision of base editing with engineered Cas9-cytidine deaminase fusions. Nat. Biotechnol. 35, 371-376 (2017). 25. Li, X. et al. Base editing with a Cpf1-cytidine deaminase fusion. Nat. Biotechnol. 36, 324-327 (2018). Gehrke, J. M. et al. An APOBEC3A-Cas9 base editor with minimized bystander and off-target activities. Nat. Biotechnol. (2018). doi:10.1038/nbt.4199. Rees, H. A. & Liu, D. R. Base editing: precision chemistry on the genome and transcriptome of living cells. Nat. Rev. Genet. 1 (2018). doi:10.1038/s41576-018-0059-1.

Accordingly, in various aspects of the disclosure, the TnpB is fused to a deaminase suitable for base editing. In some embodiments, the deaminase is selected from an adenosine deaminase, E. coli tRNA adenosine, or TadA deaminase wherein TadA is engineered for higher efficiency in human cells in comparison to pWT TadA base editor. In certain embodiments, TadA is engineered through directed evolution.

In certain other embodiments, the deaminase comprises a cytidine deaminase. Preferably, the cytidine deaminase is engineered for higher efficiency in human cells in comparison to wild type cytidine deaminase base editor. In further embodiments, the TnpB genome editing system contains one or more uracil glycosylase inhibitor.

In yet other embodiments, the TnpB-deaminase fusions are linked using a polypeptide comprising glycine and serine residues or unstructured XTEN protein polymer.

In further embodiments, the TnpB RuvC domain is mutated wherein the mutation slows cleavage of the target strand or slows the cleavage of the non-target strand. In other embodiments, the TnpB is mutated to be catalytically inactive.

In certain preferred embodiments one or more deaminase is fused to a TnpB dimer. In certain embodiments, the deaminase is fused to the N-terminus of TnpB. In other embodiments, the deaminase is fused to the C-terminus of TnpB. In further embodiments, the deaminase is placed in various locations of the TnpB including without limitations: inside the Rec-domain of the TnpB, after the Rec-domain of the TnpB, in the Wedge domain of TnpB, after the Wedge domain of TnpB, in the RuvC domain of TnpB, after the RuvC domain of TnpB, in the Helical hairpin domain of TnpB, after the Helical hairpin domain of TnpB, in the ZnF domain of TnpB, after the Znf domain of TnpB. The present disclosure contemplates placement of the deaminase in and around or near or adjacent to the aforementioned domains.

In certain alternative embodiments, the TnpB fusion protein is co-expressed with one or more TnpB not fused to a deaminase. In other embodiments, the unfused TnpB is mutated to be catalytically inactive. In other examples, the TnpB fusion contains one or more nuclear localization signals selected or derived from SV40, c-Myc or NLP-1.

In other exemplary embodiments, the TnpB-deaminase fusions bind to a guide RNA or a reRNA. In instances where the TnpB system is fused to a polypeptide that modulates host-repair. In some examples, the polypeptide is a uracil glycosylase inhibitor. In other examples, the polypeptide inhibits mismatch repair wherein the MMR inhibiting polypeptide is a dominant negative MLH1.

In various other aspects, one or more TnpB is fused to a reverse transcriptase suitable for prime editing. In some embodiments, the reverse transcriptase comprises M-MLV. In certain embodiments, the M-MLV is an engineered reverse transcriptase variant designed to improve processivity, efficiency, and/or fidelity. In various embodiments, the reverse transcriptase is derived from the human genome or derived from a human endogenous retrovirus.

In one embodiment, the accessory function that is added or otherwise coupled or attached to a TnpB polypeptide (e.g., deaminase or reverse transcriptase) provides for a TnpB-based system that is capable of performing a specialized function or activity (e.g., base editing or prime editing). For example, the TnpB protein may be fused, operably coupled to, or otherwise associated with one or more heterologous functionals domains. In certain example embodiments, the TnpB protein may be a catalytically dead TnpB protein and/or have nickase activity. A nickase is an TnpB protein that cuts only one strand of a double stranded target. In such embodiments, the catalytically inactive TnpB or nickase provide a sequence specific targeting functionality via the coRNA that delivers the functional domain to or proximate a target sequence.

It is also contemplated that the TnpB complex as a whole may be associated with two or more functional domains. For example, there may be two or more functional domains associated with the TnpB polypeptide, or there may be two or more functional domains associated with the reRNA component (via one or more adaptor proteins or aptamers), or there may be one or more functional domains associated with the TnpB polypeptide and one or more functional domains associated with the reRNA component.

In one embodiment, one or more functional domains are associated with a TnpB polypeptide via an adaptor protein, for example as used with the modified guides of Konnerman et al. (Nature 517, 583-588, 29 Jan. 2015). In one embodiment, the one or more functional domains is attached to the adaptor protein so that upon binding of the TnpB polypeptide to reRNA and target, the functional domain is in a spatial orientation allowing for the functional domain to function in its attributed function.

Exemplary functional accessory domains that may be fused to, operably coupled to, or otherwise associated with an TnpB protein can be or include, but are not limited to a nuclear localization signal (NLS) domain, a nuclear export signal (NES) domain, a translational activation domain, a transcriptional activation domain (e.g. VP64, p65, MyoDl, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, a ligase domain, a topoisomerase domain, a deaminase domain, a polymerase domain (e.g., reverse transcriptase), an integrase domain, and combinations thereof. In an embodiment, the functional domain is an HNH domain, and may be used with a naturally catalytically inactive TnpB protein to engineer a nickase. Methods for generating catalytically dead TnpB or a nickase TnpB can be adapted from approaches in Cas9 proteins, see, for example, WO 2014/204725, Ran et al. Cell. 2013 Sep. 12; 154(6): 1380-1389, known in the art and incorporated herein by reference. Briefly, one or more mutations in the catalytic domain of the RuvC domain and/or the HNH domain of the TnpB protein can be introduced that may reduce or abolish NHEJ activity. In an aspect, at least one mutation in the RuvC domain and at least one mutation in the HNH domain is provided. In an embodiment, the TnpB polypeptide comprises a mutation at D191 and/or E278 based on amino acid sequence positions of Deinococcus radiodurans ISDra2. In an aspect, the amino acid mutations comprise D191A and/or E278A based on amino acid sequence positions of Deinococcus radiodurans ISDra2.

In one embodiment, the functional domains can have one or more of the following activities: nucleobase deaminse activity, reverse transcriptase activity, retrotransposase activity, transposase activity, integrase activity, recombinase activity, topoisomerase activity, ligase activity, polymerase activity, helicase activity, methylase activity, demethylase activity, translation activation activity, translation initiation activity, translation repression activity, transcription activation activity, transcription repression activity, transcription release factor activity, histone modification activity, nuclease activity (e.g. VirD2), single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In one embodiment, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) betagalactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the TnpB protein. In embodiments having two or more functional domains, each of the two can be positioned at or near or in proximity to a terminus of the TnpB protein. In one embodiment, such as those where the functional domain is operably coupled to the effector protein, the one or more functional domains can be tethered or linked via a suitable linker (including, but not limited to, GlySer linkers) to the TnpB protein. When there is more than one functional domain, the functional domains can be same or different. In one embodiment, all the functional domains are the same. In one embodiment, all of the functional domains are different from each other. In one embodiment, at least two of the functional domains are different from each other. In one embodiment, at least two of the functional domains are the same as each other.

In additional embodiments, the TnpB-deaminase fusion protein is co-expressed with a TnpB not fused to a reverse transcriptase. Preferably, the unfused TnpB is mutated to be catalytically inactive, however, fused TnpB may also be mutated to be catalytically inactive, either or both. Various TnpB-RT fusion protein binds to a truncated reRNA or to a truncated guide RNA. In some embodiments, this maintains DNA binding activity but slows cleavage kinetics or deactivates DNA cleavage partially or entirely. Additional embodiments, include the reverse transcriptase fused to the N-terminus of TnpB or to the C-terminus of TnpB. In further embodiments, the reverse transcriptase is placed inside the Rec-domain of the TnpB, after the Rec-domain of the TnpB, in the Wedge domain of TnpB, after the Wedge domain of TnpB, in the RuvC domain of TnpB, after the RuvC domain of TnpB, in the Helical hairpin domain of TnpBafter the Helical hairpin domain of TnpB, in the ZnF domain of TnpB, after the Znf domain of TnpB.

Preferably, the TnpB-RT fusion protein is bound to an engineered reRNA wherein the engineered reRNA contains a 5′ extension, the engineered reRNA contains a 3′ extension, the extensions contain a template for a desired edit, the extension contains homology to the target site, the extension contains homology to the human genome, the extension contains sequence encoding a landing-pad for a homing integrase and/or recombinase. In preferred embodiments, the TnpB-RT fusion protein is fused or cleaved. In certain embodiments, the TnpB-RT system is fused to a polypeptide that modulates host-repair, wherein the polypeptide is a uracil glycosylase inhibitor, wherein the polypeptide inhibits mismatch repair, wherein the MMR inhibiting polypeptide is a dominant negative MLH1.

In various aspects of the disclosure, the TnpB fused to a transcriptional modulating polypeptide suitable for transcriptional interference, activation or epigenetic editing.

In some embodiments, the TnpB-transcriptional modulating polypeptide fusions comprise one or more nuclear localization signals selected or derived from SV40, e-Myc or NLP-1.

In other embodiments, the TnpB-transcriptional modulating polypeptide fusion proteins bind to a truncated guide RNA. In further embodiments, the TnpB-transcriptional modulating polypeptide comprises glycine and serine residues. In yet other embodiments, the TnpB-transcriptional modulating polypeptide are linked to one or more unstructured XTEN protein polymers.

In various embodiments, the transcriptional modulating polypeptide of the TnpB-transcriptional modulating polypeptide fusion performs histone acetylation or comprises histone acetyltransferase (HAT) p300 activity.

In other embodiments, the transcriptional modulating polypeptide of the TnpB-transcriptional modulating polypeptide fusion performs histone demethylation or comprises lysine-specific demethylase (LSD1) activity.

In further embodiments, the transcriptional modulating polypeptide of the TnpB-transcriptional modulating polypeptide fusion performs cystine methylation or comprises one or more activities selected from DNA (cytosine-5)-methyltransferase (DNMT3A), DNA-methyltransferase 3-like (DNMT3L) and MQ1.

In other embodiments, the transcriptional modulating polypeptide of the TnpB-transcriptional modulating polypeptide fusion performs cystine demethylation or comprises TET1 activity.

In additional embodiments, the transcriptional modulating peptide of the TnpB-transcriptional modulating polypeptide fusion is a transcriptional repressor or comprises a KRAB domain.

Alternatively, the transcriptional modulating peptide of the TnpB-transcriptional modulating polypeptide fusion is a transcriptional activator or comprises one or more activators including without limitation, for example, HS1, VP64 and p65.

In other embodiments, Where the the transcriptional modulating peptide of the TnpB-transcriptional modulating polypeptide fusion is a repressor or comprises multiple transcriptional modulating peptides. In yet other embodiments, the TnpB of the TnpB-transcriptional modulating polypeptide fusion is mutated to be catalytically inactive.

In further embodiments, the transcriptional modulating peptides of the TnpB-transcriptional modulating polypeptide fusion are physically coupled through an engineered reRNA wherein the reRNA comprises one or more aptamers.

In additional embodiments, the transcriptional modulating peptides of the TnpB-transcriptional modulating polypeptide fusion are physically coupled through an engineered guide RNA, wherein the guide RNA contains one or more aptamers.

reRNA

The TnpB systems herein may further comprise one or more nucleic acid components, which are also referred to herein as reRNA. As reported in Karvelis et al., “Transposon-associated TnpB is a programmable RNA-guided DNA endonuclease,” Nature, Nov. 25, 2021, Vol. 599, pp. 692-700 (incorporated herein by reference), TnpB is an RNA-guided dsDNA nuclease that forms a complex with a non-coding RNA called “reRNA.” The reRNA is a transcript that is generated from the transcription of the IS DNA sequence beginning at a transcription initiation site located within the 3′ end of the TnpB coding region and ending at a transcription termination site located in the flanking genomic DNA region that is immediately downstream of the RE of the Insertion Sequence. Thus, the reRNA comprises three regions: (a) a region corresponding to the 3′ end of the TnpB coding region, (b) a region corresponding to the RE, and (c) a region corresponding to the flanking genomic DNA immediately downstream of the 3′ end of the RE. Regions (a) and (b) generally form a folded scaffold that appears to bind to the TnpB protein. Region (c) functions as a spacer or targeting sequence which allows for the targeting of a TnpB-reRNA complex to a target site to which the region (c) has complementarity to and anneals. Region (c), in various embodiments, can be engineered to be any desired target sequence such that the TnpB-reRNA complex is targeted to a desired target sequence.

Thus, the reRNA sequence may be predicted from the sequence of the region spanning the 3′ end of the TnpB coding region through a flanking region downstream of the RE.

Computational methods can be used to predict the reRNA sequences for identified TnpB and TnpB-like proteins. As reported in Karvelis et al., “Transposon-associated TnpB is a programmable RNA-guided DNA endonuclease,” Nature, Nov. 25, 2021, Vol. 599, pp. 692-700, the TnpB protein co-purified with an RNA molecule of about 150 nucleotides long which had a sequence that was derived from the IS and a sequence downstream of the IS.

In various embodiments, reRNA may be engineered to include RNA, DNA, or combinations of both and include modified and non-canonical nucleotides as described further below. The reRNA can comprise a reprogrammable spacer sequence and a scaffold that interacts with the TnpB polypeptide. reRNA may form a complex with a TnpB polypeptide, and direct sequence-specific binding of the complex to a target sequence of a target polynucleotide. In one example embodiment, the reRNA is a single molecule comprising a scaffold sequence and a spacer sequence. In certain example embodiments, the spacer is 5′ of the scaffold sequence. In one example embodiment, the reRNA may further comprise a conserved nucleic acid sequence between the scaffold and spacer portions.

In embodiments, the reRNA comprises a spacer sequence and a scaffold sequence, e.g. a conserved nucleotide sequence. In embodiments, the reRNA comprises about 45 to about 350 nucleotides, or about 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 17, 138, 19, 140, 141, 142, 143, 144, 145, 146, 147, 148, 149, 150, 151, 152, 153, 154, 155, 156, 157, 158, 159, 160, 11, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199,200, 201, 202,203,204, 205, 206,207,208,209,210, 211, 212,213, 214,215, 216, 217, 218, 219, 220, 221, 222, 223, 224, 225, 226, 227, 228, 229, 230, 231, 232, 233, 234, 235, 236, 237, 238, 239, 2340, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 272, 273, 273, 274, 275, 276, 277, 278, 279, 280, 281, 282,283,284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 333, 334, 335, 336, 337, 338, 339, 340, 341, 342, 343, 344, 345, 346, 347, 348, 349, or 350 nucleotides.

In embodiments, the reRNA comprises a scaffold sequence, e.g. a conserved nucleotide sequence that binds to the TnpB protein. The scaffold sequence therefore typically comprises conserved regions, with the scaffold comprising about 30 to 200 nucleotides, about 50 to 180, about 80 to 175 nucleotides, or about 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or more nucleotides.

The reRNA may further comprise a spacer, which can be re-programmed to direct site specific binding to a target sequence of a target polynucleotide. The spacer may also be referred to herein as part of the reRNA scaffold or reRNA, and may comprise an engineered heterologous sequence.

In one embodiment, the spacer length or targeting sequence length of the reRNA is from 10 to 50 nt. In one embodiment, the spacer length of the oRNA is at least 10, 11, 12, 13, 14, or 15 nucleotides. In one embodiment, the spacer length is from 10 to 40 nucleotides, from 15 to 30 nt, 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In example embodiments, the spacer sequence is 14, 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, or 50 nt.

As used herein, the term “spacer” may also be referred to as a “guide sequence” or “targeting sequence” which has complementarity to a target sequence (e.g., a desired target gene in a genome which is desired to be edited). In one embodiment, the degree of complementarity of the spacer sequence to a given target sequence, when optimally aligned using a suitable alignment algorithm, is about or more than 50%, 60%, 75%, 80%, 85%, 90%, 95%, 97.5%, 99%, or more. In certain example embodiments, the reRNA molecule comprises a spacer sequence that may be designed to have at least one mismatch with the target sequence, such that a RNA duplex formed between the sequence and the target sequence. Accordingly, the degree of complementarity is less than 99%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (Illumina, San Diego, CA), SOAP (available at soap.genomics.org.cn), and Maq (available at maq.sourceforge.net).

The ability of a sequence (within a nucleic acid-targeting reRNA molecule) to direct sequence-specific binding of a nucleic acid-targeting complex to a target nucleic acid sequence may be assessed by any suitable assay. For example, the components of a reRNA system sufficient to form a TnpB-targeting complex, including the reRNA molecule sequence to be tested, may be provided to a host cell having the corresponding target nucleic acid sequence, such as by transfection with vectors encoding the components of the TnpB-targeting complex, followed by an assessment of preferential targeting (e.g., cleavage) within the target nucleic acid sequence. Similarly, cleavage of a target nucleic acid sequence (or a sequence in the vicinity thereof) may be evaluated in a test tube by providing the target nucleic acid sequence, components of a TnpB-targeting complex, including the sequence to be tested and a control sequence different from the test coRNA, and comparing binding or rate of cleavage at or in the vicinity of the target sequence between the test and control reRNA molecule sequence reactions. Other assays are possible, and will occur to those skilled in the art. A spacer sequence, and hence a nucleic acid targeting reRNA may be selected to target any target nucleic acid sequence.

reRNA Modifications

In one embodiment, the reRNA comprises non-naturally occurring nucleic acids and/or non-naturally occurring nucleotides and/or nucleotide analogs, and/or chemical modifications. Preferably, these non-naturally occurring nucleic acids and non-naturally occurring nucleotides are located outside the reRNA sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a reRNA component nucleic acid comprises ribonucleotides and non-ribonucleotides. In one such embodiment, a reRNA component comprises one or more ribonucleotides and one or more deoxyribonucleotides. In an embodiment of the disclosure, the reRNA component comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA).

Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of coRNA chemical modifications include, without limitation, incorporation of 2′-O-methyl (M), 2′-O-methyl 3′ phosphorothioate (MS), S-constrained ethyl(cEt), or 2′-O-methyl 3′thioPACE (MSP) at one or more terminal nucleotides. Such chemically modified oRNA components can comprise increased stability and increased activity as compared to unmodified oRNA components, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112: 11870-11875; Sharma et al., MedChemComm., 2014, 5: 1454-1471; Hendel et al., Nat. Biotechnol. (2015) 33(9): 985-989; Li et al., Nature Biomedical Engineering, 2017, 1, 0066 D01: 10.1038/s41551-017-0066). In one embodiment, the 5′ and/or 3′ end of a reRNA component is modified by a variety of functional moieties including fluorescent dyes, polyethylene glycol, cholesterol, proteins, or detection tags. (See Kelly et al., 2016, J. Biotech. 233:74-83). In one embodiment, a reRNA component comprises ribonucleotides in a region that binds to a target sequence and one or more deoxyribonucletides and/or nucleotide analogs in a region that binds to the TnpB polypeptide.

In an embodiment, deoxyribonucleotides and/or nucleotide analogs are incorporated in engineered reRNA component structures. In one embodiment, 3-5 nucleotides at either the 3′ or the 5′ end of a reRNA component is chemically modified. In one embodiment, only minor modifications are introduced in the seed region, such as 2′-F modifications. In one embodiment, 2′-F modification is introduced at the 3′ end of a reRNA component. In one embodiment, three to five nucleotides at the 5′ and/or the 3′ end of the reRNA component are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In one embodiment, all of the phosphodiester bonds of a reRNA component are substituted with phosphorothioates (PS) for enhancing levels of gene disruption. In one embodiment, more than five nucleotides at the 5′ and/or the 3′ end of the reRNA component are chemically modified with 2′-O-Me, 2′-F or S-constrained ethyl(cEt). Such chemically modified reRNA component can mediate enhanced levels of gene disruption (see Ragdarm et al., 0215, PNAS, E7110-E7111). In an embodiment of the disclosure, a reRNA component is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the reRNA component by a linker, such as an alkyl chain. In one embodiment, the chemical moiety of the modified nucleic acid component can be used to attach the reRNA component to another molecule, such as DNA, RNA, protein, or nanoparticles. Such chemically modified reRNA component can be used to identify or enrich cells generically edited by a TnpB polypeptide and related systems (see Lee et al., eLife, 2017, 6:e25312, DOI: 10.7554).

Other reRNA modifications are described in Kim, D. Y., Lee, J. M., Moon, S. B. et al. Efficient CRISPR editing with a hypercompact Cas12f1 and engineered guide RNAs delivered by adeno-associated virus. Nat Biotechnol 40, 94-102 (2022).

Accordingly, in various aspects of the disclosure, the reRNA are modified in one or more TnpB reRNA. MS1, an internal penta(uridinylate) (UUUUU) sequence in the tracrRNA; MS2, the 3′ terminus of the crRNA; MS3, the ‘stem 1’ region of the tracrRNA; MS4, the traerRNA-crRNA complementary region; and MS5, the ‘stem 2’ region of the tracrRNA.

Various aspects of the disclosure provide methods and compositions for improved reRNA stability via chemical modifications. Braasch, D. A., Jensen, S., Liu, Y., Kaur, K., Arar, K., White, M. A., et al. (2003). RNA interference in mammalian cells by chemically-modified RNA. Biochemistry 42, 7967-7975. Chiu, Y. L., and Rana, T. M. (2003). siRNA function in RNAi: a chemical modification analysis. RNA 9, 1034-1048. Behlke, M. A. (2008). Chemical modification of siRNAs for in vivo use. Oligonucleotides 18, 305-319. Bennett, C. F., and Swayze, E. E. (2010). RNA targeting therapeutics: molecular mechanisms of antisense oligonucleotides as a therapeutic platform. Annu. Rev. Pharmacol. Toxicol. 50, 259-293. Deleavey, G. F., and Damha, M. J. (2012). Designing chemically modified oligonucleotides for targeted gene silencing. Chem. Biol. 19, 937-954. Lennox, K. A., and Behlke, M. A. (2020). Chemical modifications in RNA interference and CRISPR/Cas genome editing reagents. Methods Mol. Biol. 2115, 23-55.

For instance, Hendel et al. improved guideRNA stability by chemically modifying gRNA ends to reduce degradation by exonucleases, RNA nuclease. Hendel, A., Bak, R. O., Clark, J. T., Kennedy, A. B., Ryan, D. E., Roy, S., et al. (2015a). Chemically modified guide RNAs enhance CRISPR-Cas genome editing in human primary cells. Nat. Biotechnol. 33, 985-989. Chemical modifications of gRNAs may enable more efficient and safer gene-editing in primary cells suitable for clinical applications.

A review of types of chemical modifications are provided in the table below. Allen, Daniel et al. “Using Synthetically Engineered Guide RNAs to Enhance CRISPR Genome Editing Systems in Mammalian Cells.” Frontiers in genome editing vol. 2 617910. 28 Jan. 2021.

| | | Effect on |
|---|---|---|
| | | genome editing |
| Modification(s) | Modification location | efficiency |
| M | Terminal residues | ↑^{#} |
| MS | Terminal residues | ↑^{#} |
| | Spacer (PAM-distal region) | ↑* |
| | Spacer (tracrRNA-binding region) | ↑* |
| | Spacer (Seed region) | ↓ |
| MSP | Terminal residues | ↑^{#} |
| cEt | Spacer (PAM-distal region) | ↑ |
| | Spacer (tracrRNA-binding) | ↑ |
| | Spacer (Seed region) | ↓ |
| 2′-F | Spacer (PAM-distal region) | ↑ |
| | Spacer (tracrRNA-binding) | ↑ |
| | Spacer (Seed region) | ↓ |
| 2′-F + PS | Spacer (PAM-distal region) | ↑ |
| | Spacer (tracrRNA-binding) | ↑ |
| | Spacer (Seed region) | ↓ |
| | Spacer (Seed region, Cas9-non- | ↑* |
| | interacting residues) | |
| PS | Whole crRNA | ↑ |

*additionally validated in vivo;
^{#}additionally validated in human primary cells
2′-O-methyl (M or 2′-O-Me); 2′-O-methyl 3′phosphorothioate (MS); 2′-O-methyl-3′-thioPACE (MSP); S-constrained ethyl (cET); 2′-fluoro (2′-F); phosphorothioate (PS)

Accordingly, in various embodiments of the present disclosure, the genome editing system comprising TnpB and further comprises one or more chemical modifications selected from, but not limited to the modifications in the above table.

In exemplary embodiments, chemical modifications to the reRNA include modifications on the ribose rings and phosphate backbone of reRNAs and modifications at the 2′OH include 2′-O-Me, 2′-F, and 2′F-ANA. More extensive ribose modifications include 2′F-4′-Cα-OMe and 2′,4′-di-Cα-OMe combine modification at both the 2′ and 4′ carbons. Phosphodiester modifications include sulfide-based Phosphorothioate (PS) or acetate-based phosphonoacetate alterations. Combinations of the ribose and phosphodiester modifications have given way to formulations such as 2′-O-methyl 3′phosphorothioate (MS), or 2′-O-methyl-3′-thioPACE (MSP), and 2′-O-methyl-3′-phosphonoacetate (MP) RNAs. Locked and unlocked nucleotides such as locked nucleic acid (LNA), bridged nucleic acids (BNA), S-constrained ethyl (cEt), and unlocked nucleic acid (UNA) are examples of sterically hindered nucleotide modifications. Modifications to make a phosphodiester bond between the 2′ and 5′ carbons (2′,5′-RNA) of adjacent RNAs as well as a butane 4-carbon chain link between adjacent RNAs have been described.

Any of the above TnpB editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### Integrase Editors (e.g., PASTE)

In some embodiments, the gene editing system comprises one or more integrase editors. In certain embodiments, the gene editing system comprises a construct enabling programmable addition via site-specific targeting elements (PASTE). In certain embodiments, the gene editing system comprises one or more integrase editors and/or gene editing systems described and disclosed in PCT Publications WO2022087235A1, WO2020191245A1, WO2022060749A1, WO2021188840A1, WO2021138469A1, US Patent Application Publications US20140349400A1, US20210222164A1 or US20150071898A1, each of which is incorporated by reference herein in their entirety. In certain embodiments, the one or more integrase editors comprise CRISPR directed integrases disclosed in Yarnall, M. T. N., Ioannidi, E. I., Schmitt-Ulms, C. et al. Drag-and-drop genome insertion of large sequences without double-strand DNA cleavage using CRISPR-directed integrases. Nat Biotechnol (2022).

### Epigenetic Editors

In still other embodiments, the LNPs may be used to deliver an epigenetic editing system. Epigenetic editors are generally composed of an epigenetic enzyme or their catalytic domain fused with a user-programmable DNA-binding protein, such as a CRISPR-Cas enzyme or TnpB enzyme. The user-programmable DNA-binding protein (plus a guide RNA in the case of a nucleic acid programmable DNA binding protein) guides the epigenetic enzyme (e.g., a DNA methyltransferase or DNMT) to a specific site (e.g., a CpG island in a promoter region of a gene) in order to induce a change in promoter activity.

Epigenetic modifications of DNA and histones are known for their multifaceted contributions to transcriptional regulation. As these modifications are faithfully propagated throughout DNA replication, they are considered central players in cellular memory of transcriptional states. Many efforts in the last decade have generated a vast understanding of individual epigenetic modifications and their contribution to transcriptional regulation. Epigenetic editing offers powerful tools to selectively induce epigenetic changes in a genome without altering the sequence of a nucleotide sequence as a means to regulate gene activity. The foundation of epigenetic editing is formed by the ability to generate fusion proteins of epigenetic enzymes or their catalytic domains with programmable DNA-binding platforms such as the clustered regularly interspaced short palindromic repeat (e.g., CRISPR Cas9 or Cas12a) to target these to an endogenous locus of choice. The enzymatic fusion protein then dictates the initial deposited modification while subsequent cross-talk within the local chromatin environment likely influences epigenetic and transcriptional output.

The following published literature discussing epigenetic editing is incorporated herein by reference each in their entireties.
Gjaltema RAF, Rots M G. Advances of epigenetic editing. Curr Opin Chem Biol. 2020 August; 57:75-81. Epub 2020 Jun. 30. PMID: 32619853.
Kleinstiver B P, Sousa A A, Walton R T, Tak Y E, Hsu J Y, Clement K, Welch M M, Horng J E, Malagon-Lopez J, Scarfó I, Maus M V, Pinello L, Aryee M J, Joung J K. Engineered CRISPR-Cas12a variants with increased activities and improved targeting ranges for gene, epigenetic and base editing. Nat Biotechnol. 2019 March; 37(3):276-282. Epub 2019 Feb. 11. Erratum in: Nat Biotechnol. 2020 July; 38(7):901. PMID: 30742127; PMCID: PMC6401248.
Rots M G, Jeltsch A. Editing the Epigenome: Overview, Open Questions, and Directions of Future Development. Methods Mol Biol. 2018; 1767:3-18. PMID: 29524127.
Liu X S, Jaenisch R. Editing the Epigenome to Tackle Brain Disorders. Trends Neurosci. 2019 December; 42(12):861-870. Epub 2019 Nov. 7. PMID: 31706628.
Waryah C B, Moses C, Arooj M, Blancafort P. Zinc Fingers, TALEs, and CRISPR Systems: A Comparison of Tools for Epigenome Editing. Methods Mol Biol. 2018; 1767:19-63. PMID: 29524128.
Xu X, Hulshoff M S, Tan X, Zeisberg M, Zeisberg E M. CRISPR/Cas Derivatives as Novel Gene Modulating Tools: Possibilities and In Vivo Applications. Int J Mol Sci. 2020 Apr. 25; 21(9):3038. PMID: 32344896; PMCID: PMC7246536.

In addition, the following published patent literature relating to epigenetic editing is incorporated herein by reference each in their entireties.

| Pub Number | Title |
|---|---|
| WO2023283359A2 | COMPOSITIONS AND METHODS FOR MODULATING SECRETED |
| | FRIZZLED RECEPTOR PROTEIN 1 (SFRP1) GENE EXPRESSION |
| WO2022226139A1 | TISSUE-SPECIFIC NUCLEIC ACID DELIVERY BY MIXED CATIONIC |
| | LIPID PARTICLES |
| WO2022132926A1 | TISSUE-SPECIFIC NUCLEIC ACID DELIVERY BY 1,2-DIOLEOYL-3- |
| | TRIMETHYLAMMONIUM-PROPANE (DOTAP) LIPID NANOPARTICLES |
| WO2021183720A1 | COMPOSITIONS AND METHODS FOR MODULATING FORKHEAD BOX |
| | P3 (FOXP3) GENE EXPRESSION |
| WO2021061815A1 | COMPOSITIONS AND METHODS FOR MODULATING HEPATOCYTE |
| | NUCLEAR FACTOR 4-ALPHA (HNF4α) GENE EXPRESSION |
| WO2021061707A1 | COMPOSITIONS AND METHODS FOR MODULATING APOLIPOPROTEIN |
| | B (APOB) GENE EXPRESSION |
| WO2021061698A1 | METHODS AND COMPOSITIONS FOR MODULATING FRATAXIN |
| | EXPRESSION AND TREATING FRIEDRICH'S ATAXIA |

### Gene Writing

In some embodiments, the gene editing system is a gene writing system. In certain embodiments, the gene editing system is one described and disclosed in US Patent Application Publications US2022039681A1 or US20200109398A1, each of which is incorporated by reference herein in their entirety.

In certain embodiments, the gene editing system is a system for modifying DNA comprising a polypeptide or a nucleic acid encoding a polypeptide capable of target primed reverse transcription, wherein the polypeptide comprises (a) a reverse transcriptase domain and (b) an endonuclease domain, wherein at least one of (a) or (b) is heterologous; and a template RNA comprising (i) a sequence that binds the polypeptide and (ii) a heterologous object sequence. In certain embodiments, the gene editing system is a system for modifying DNA comprising a polypeptide or a nucleic acid encoding a polypeptide capable of target primed reverse transcription, wherein the polypeptide comprises (a) a target DNA binding domain, (b) a reverse transcriptase domain and (c) an endonuclease domain, wherein at least one of (a), (b) or (c) is heterologous, and a template RNA comprising (i) a sequence that binds the polypeptide and (ii) a heterologous object sequence. In certain embodiments, the polypeptide comprises a sequence of at least 50 amino acids having at least 80% identity to a reverse transcriptase domain of a sequence of a polypeptide listed in TABLE 1, TABLE 2, or TABLE 3 of US Patent Application Publication US20200109398A1, which is incorporated by reference in its entirety, including the aforementioned sequence tables.

In certain embodiments, the reverse transcriptase domain is from a retrovirus or a retrotransposon, such as a LTR-retrotransposon, or a non-LTR retrotransposon. In certain embodiments, the reverse transcriptase is from a non-LTR retrotransposon, wherein the non-LTR retrotransposon is a RLE-type non-LTR retrotransposon from the R2, NeSL, HERO, R4, or CRE clade, or an APE-type non-LTR retrotransposon from the R1, or Tx1 clade. In certain embodiments, the reverse transcriptase domain is from an avian retrotransposase of column 8 of Table 3 of US20200109398A1, or a sequence having at least 70%, identity thereto. In certain embodiments, the reverse transcriptase domain does not comprise an RNA binding domain and the polypeptide comprises an RNA binding domain heterologous to the reverse transcriptase domain, wherein the RNA binding domain is a B-box protein, a MS2 coat protein, a dCas protein, or a UTR binding protein, or a fragment or variant of any of the foregoing.

In certain embodiments, the endonuclease domain is heterologous to the reverse transcriptase domain, and wherein the endonuclease is a Fok1 nuclease (or a functional fragment thereof), a type-II restriction 1-like endonuclease (RLE-type nuclease), another RLE-type endonuclease, or a Prp8 nuclease. In certain embodiments, the endonuclease domain is heterologous to the reverse transcriptase domain, wherein endonuclease domain contains DNA binding functionality. In certain embodiments, the endonuclease domain is heterologous to the reverse transcriptase domain, and wherein the endonuclease has nickase activity and does not form double stranded breaks.

In certain embodiments, the polypeptide comprises a DNA binding domain heterologous to the reverse transcriptase domain, and wherein the DNA binding domain is: a zinc-finger element, or a functional fragment thereof; or a TAL effector element, or a functional fragment thereof; a Myb domain; or a sequence-guided DNA binding element. In certain embodiments, the polypeptide comprises a DNA binding domain heterologous to the reverse transcriptase domain, and wherein the DNA binding element is a sequence-guided DNA binding element, further wherein the sequence-guided DNA binding element is Cas9, Cpf1, or other CRISPR-related protein. In certain embodiments, the polypeptide comprises a DNA binding domain heterologous to the reverse transcriptase domain, and wherein the DNA binding domain is a transcription factor.

In certain embodiments, the sequence-guided DNA binding element has been altered to have no endonuclease activity. In certain embodiments, the sequence-guided DNA binding element replaces the endonuclease element of the polypeptide. In certain embodiments, the editing system is capable of modifying DNA using reverse transcriptase activity, optionally in the absence of homologous recombination activity.

In certain embodiments, the gene editing system is a system for modifying DNA comprising: a) a recombinase polypeptide selected from Rec27 (WP_021170377.1, SEQ ID NO: 1241 of US20220396813A1), Rec35 (WP_134161939.1, SEQ ID NO: 1249 of US20220396813A1), or comprising an amino acid sequence of Table 1 or 2 of US20220396813A1, or an amino acid sequence having at least 70%, 75%, 80%, 85%, 90%, 95%, 96%, 97%, 98%, or 99% identity thereto, or a nucleic acid encoding the recombinase polypeptide; and
b) a double-stranded insert DNA comprising:
   (i) a DNA recognition sequence that binds to the recombinase polypeptide of (a), said DNA recognition sequence having a first parapalindromic sequence and a second parapalindromic sequence, wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1 of US20220396813A1, or a nucleotide sequence having at least 70%, 75%, 80%, 85%, 90%, 95%, 96%, 97%, 98%, or 99% identity thereto, or having no more than 1, 2, 3, 4, 5, 6, 7, 8 sequence alterations (e.g., substitutions, insertions, or deletions) relative thereto, and said DNA recognition sequence further comprises a core sequence of about 5-10 nucleotides, e.g., about 8 nucleotides, wherein the core sequence is situated between the first and second parapalindromic sequences, and
   (ii) a heterologous object sequence.

### Gene Inactivating Systems

In some embodiments, the gene editing system comprises a polypeptide or an RNA encoding a polypeptide capable of inducing a double-stranded or single-stranded break in a desired gene, thereby inactivating said gene. In certain embodiments, the gene editing system is one described and disclosed in PC Publications WO2020028327A1, WO2020069296A1 or WO2020118041A1, each of which is incorporated by reference herein in their entirety. In certain embodiments, the gene editing system is one described and disclosed in a patent application publication disclosed below, each of which is incorporated by reference herein in their entirety:

| Pub Number | Title |
|---|---|
| WO2022271780A1 | METHODS FOR IN VIVO EDITING OF A LIVER GENE |
| WO2022261292A1 | MODIFIED GUIDE RNAS COMPRISING AN INTERNAL LINKER FOR |
| | GENE EDITING |
| WO2022221697A1 | LIPID NANOPARTICLE COMPOSITIONS |
| WO2022221696A1 | INHIBITORS OF DNA-DEPENDENT PROTEIN KINASE AND |
| | COMPOSITIONS AND USES THEREOF |
| WO2022221695A1 | LIPID NANOPARTICLE COMPOSITIONS |
| WO2022170194A2 | LYMPIIOCYTE ACTIVATION GENE 3 (LAG3) COMPOSITIONS AND |
| | METHODS FOR IMMUNOTHERAPY |
| WO2022170193A2 | T-CELL IMMUNOGLOBULIN AND MUCIN DOMAIN 3 (TIM3) |
| | COMPOSITIONS AND METHODS FOR IMMUNOTHERAPY |
| WO2022170172A1 | NATURAL KILLER CELL RECEPTOR 2B4 COMPOSITIONS AND |
| | METHODS FOR IMMUNOTHERAPY |
| WO2022147133A1 | ENGINEERED T CELLS |
| WO2022140587A1 | COMPOSITIONS AND METHODS FOR GENETICALLY MODIFYING |
| | CIITA IN A CELL |
| WO2022140586A2 | COMPOSITIONS AND METHODS FOR REDUCING HLA-A IN A CELL |
| WO2022125982A1 | COMPOSITIONS AND METHODS FOR REDUCING MHC CLASS II IN |
| | A CELL |
| WO2022125968A1 | POLYNUCLEOTIDES, COMPOSITIONS, AND METHODS FOR |
| | GENOME EDITING INVOLVING DEAMINATION |
| WO2021222287A2 | METHODS OF IN VITRO CELL DELIVERY |
| WO2021220132A1 | METHODS AND COMPOSITIONS FOR OCULAR CELL THERAPY |
| WO2021188840A1 | METHODS AND COMPOSITIONS FOR DIRECTED GENOME EDITING |
| WO2021158858A1 | COMPOSITIONS AND METHODS FOR KALLIKREIN (KLKB1) GENE |
| | EDITING |
| WO2021119275A1 | MODIFIED GUIDE RNAS FOR GENE EDITING |
| WO2020219876A1 | IONIZABLE AMINE LIPIDS AND LIPID NANOPARTICLES |
| WO2020198706A1 | COMPOSITIONS AND METHODS FOR TTR GENE EDITING AND |
| | TREATING ATTR AMYLOIDOSIS COMPRISING A |
| | CORTICOSTEROID OR USE THEREOF |
| WO2020198697A1 | COMPOSITIONS AND METHODS COMPRISING A TTR GUIDE RNA |
| | AND A POLYNUCLEOTIDE ENCODING AN RNA-GUIDED DNA |
| | BINDING AGENT |
| WO2020198641A2 | POLYNUCLEOTIDES, COMPOSITIONS, AND METHODS FOR |
| | POLYPEPTIDE EXPRESSION |
| WO2020118041A1 | MODIFIED AMINE LIPIDS |
| WO2020084580A1 | METHODS AND COMPOSITIONS FOR OCULAR CELL THERAPY |
| WO2020082047A1 | COMPOSITIONS AND METHODS FOR TREATING ALPHA-1 |
| | ANTITRYPSIN DEFICIENCEY |
| WO2020082046A2 | COMPOSITIONS AND METIIODS FOR EXPRESSING FACTOR IX |
| WO2020082042A2 | COMPOSITIONS AND METHODS FOR TRANSGENE EXPRESSION |
| | FROM AN ALBUMIN LOCUS |
| WO2020082041A1 | NUCLEIC ACID CONSTRUCTS AND METHODS OF USE |
| WO2020081613A1 | COMPOSITIONS AND METHODS FOR IMMUNOTHERAPY |
| WO2020072605A1 | IONIZABLE AMINE LIPIDS |
| WO2020069296A1 | COMPOSITIONS AND METHODS FOR LACTATE DEHYDROGENASE |
| | (LDHA) GENE EDITING |
| WO2020028327A1 | COMPOSITIONS AND METHODS FOR HYDROXYACID OXIDASE 1 |
| | (HAO1) GENE EDITING FOR TREATING PRIMARY |
| | HYPEROXALURIA TYPE 1 (PH1) |
| WO2019237069A1 | MODIFIED GUIDE RNAS FOR GENE EDITING |
| WO2019237035A1 | COMPOSITIONS AND METHODS FOR IMMUNOONCOLOGY |
| WO2019067999A1 | IN VITRO METHOD OF MRNA DELIVERY USING LIPID |
| | NANOPARTICLES |
| WO2019067992A1 | FORMULATIONS |
| WO2019067910A1 | POLYNUCLEOTIDES, COMPOSITIONS, AND METHODS FOR |
| | GENOME EDITING |
| WO2019067872A1 | COMPOSITIONS AND METHODS FOR TTR GENE EDITING AND |
| | TREATING ATTR AMYLOIDOSIS |
| WO2019036513A1 | STABILIZED NUCLEIC ACIDS ENCODING MESSENGER |
| | RIBONUCLEIC ACID (MRNA) |
| WO2019003193A1 | METHODS FOR THE TREATMENT OF DISEASE WITH GENE |
| | EDITING SYSTEMS |
| WO2018175636A2 | COMPOSITIONS AND METHODS FOR IMMUNOONCOLOGY |
| WO2018142364A1 | COMPOSITIONS AND METHODS FOR THE TREATMENT OF |
| | HEMOGLOBINOPATHIES |
| WO2018119182A1 | COMPOSITIONS AND METHODS FOR TREATING ALPHA-1 |
| | ANTITRYPSIN DEFICIENCY |
| WO2018107028A1 | MODIFIED GUIDE RNAS |
| WO2018053070A1 | IMPROVED METHODS FOR ANALYZING EDITED DNA |
| WO2017185054A1 | COMPOSITIONS AND METHODS FOR TREATMENT OF DISEASES |
| | ASSOCIATED WITH TRINUCLEOTIDE REPEATS IN |
| | TRANSCRIPTION FACTOR FOUR |
| WO2017173054A1 | LIPID NANOPARTICLE FORMULATIONS FOR CRISPR/CAS |
| | COMPONENTS |
| WO2017160752A1 | METHODS AND COMPOSITIONS FOR GENE EDITING |
| WO2017115268A1 | COMPOSITIONS AND METHODS FOR THE TREATMENT OF |
| | HEMOGLOBINOPATHIES |
| WO2017093969A1 | COMPOSITIONS AND METHODS FOR IMMUNOONCOLOGY |

Compositions that Increase Gene Editing Efficiency

In some embodiments, the gene editing system comprises a polypeptide, or a nucleic acid that encodes a polypeptide, that increases gene editing efficiency. In some embodiments, the gene editing system comprises a composition described and disclosed in US Application Publication US20220090064A1, which is incorporated by reference herein in its entirety. In some embodiments, the composition comprises a guide nucleic acid, a Cas9 nickase, and/or a reverse transcriptase. The reverse transcriptase may be fused to the Cas9 nickase. The reverse transcriptase may heterodimerize with the Cas9 nickase. The reverse transcriptase may bind to a guide nucleic acid. The reverse transcriptase may be engineered to increase processivity. The guide nucleic acid may be engineered to facilitate synthesis or editing of a sequence. The guide nucleic acid may comprise a region that binds to another region on the guide nucleic acid to improve gene editing.

In some embodiments, the composition comprises a Cas 9 nickase and a reverse transcriptase, or one or two polynucleotides encoding the Cas 9 nickase and reverse transcriptase, wherein:
(i) the composition comprises a first polypeptide chain comprising the Cas nickase or a segment of the Cas nickase, and a second polypeptide chain comprising the reverse transcriptase, or the one or two polynucleotides encoding the polypeptide chains, wherein the polypeptide chains comprise leucine zippers that bind one another, or
(ii) the composition comprises a first polypeptide chain comprising a first segment of the Cas nickase, and a second polypeptide chain comprising a second segment of the Cas nickase and the reverse transcriptase, or the one or two polynucleotides encoding the polypeptide chains, wherein the polypeptide chains comprise inteins that bind one another, the Cas nickase comprises an amino acid sequence at least 80% identical to SEQ ID NO: 32 of US20220090064A1, the first and second polypeptide chains respectively comprise amino acids 1-1124 and 1125-1368 of the Cas nickase, 1-1129 and 1130-1368 of the Cas nickase, 1-1139 and 1140-1368 of the Cas nickase, 1-1167 and 1168-1368 of the Cas nickase, 1-1172 and 1173-1368 of the Cas nickase, or 1-1202 and 1203-1368 of the Cas nickase, and the Cas nickase comprises a mutation at amino acid position 1030 or after amino acid position 1030 with regard to SEQ ID NO: 32 of US20220090064A1, the mutation comprising a point mutation to a cysteine, threonine, alanine, or serine, or an insertion of a cysteine, threonine, alanine, or serine at the C-terminal half of the Cas9 nickase or
(iii) the reverse transcriptase comprises a Moloney leukemia virus reverse transcriptase (mlvRT) comprising an amino acid sequence at least 80% identical to SEQ ID NO: 13 of US20220090064A1 or at least 80% identical to a functional fragment thereof comprising at least 400 amino acids, and a point mutation at amino acid position Q84, L139, Q221, V223, T664, or L671 with regard to SEQ ID NO: 13 of US20220090064A1; wherein the respective SEQ ID NOs are those disclosed in US Application Publication US20220090064A1.

In certain embodiments, the composition comprises a guide nucleic acid comprising: optionally, a spacer reverse complementary to a first region of a target nucleic acid, wherein the spacer is included in the guide nucleic acid, or the spacer is included in a second, different guide nucleic acid when not included in the guide nucleic acid; a scaffold configured to bind to a Cas nuclease; a reverse transcriptase template encoding a sequence to be reverse transcribed into a first synthesized strand to be inserted into the target nucleic acid; a first strand primer binding site reverse complementary to a second region of the target nucleic acid; and at least one of:
(i) a guide nucleic acid positioning system (GPS) region and a GPS binding site that hybridizes to the GPS region, wherein the GPS region and the GPS binding site are at least 10 nucleotides in length and are at least 60% reverse complementary to each other, and wherein hybridization of the GPS region and the GPS binding site positions the first strand primer binding site closer to the second region of the target nucleic acid,
(ii) a GPS region that hybridizes to a GPS binding site on the second guide nucleic acid, wherein the GPS region and the GPS binding site are at least 10 nucleotides in length and are at least 60% reverse complementary to each other, wherein the second region of the target nucleic acid does not include any part of the first region of the target nucleic acid, and wherein the second region of the target nucleic acid does not include any part of a reverse complement of the first region of the target nucleic acid, and wherein hybridization of the GPS region and the GPS binding site positions the first strand primer binding site closer to the second region of the target nucleic acid, or
(iii) a modification in the reverse transcriptase template that disrupts a track of at least 4 consecutive nucleotides of the same base in the target nucleic acid.

### Zinc Finger Nucleases, TALENS, and Meganucleases

In some embodiments, the gene editing systems contemplated herein may comprise user-programmable DNA binding proteins that bind DNA through a specific amino acid sequence (i.e., are not reliant upon a guide RNA or nucleic acid programmability). Such enzymes include zinc finger nucleases and TALENS.

In some embodiments, the user-programmable nuclease is or comprises a TALE Nuclease, a TALE nickase, Zinc Finger (ZF) Nuclease, ZF Nickase, meganuclease, or a combination thereof. In some embodiments, the non-CRISPR/Cas sequence-specific nuclease is or includes two, three, four, or more of an independently selected TALE Nuclease, TALE nickase, Zinc Finger (ZF) Nuclease, ZF Nickase, Meganuclease, restriction enzymes or a combination thereof. In some embodiments, the combination is or comprises a TALE Nuclease/a ZF Nuclease; a TALE Nickase/a ZF nickase.

### TALENs

In some embodiments, the non-CRISPR/Cas sequence-specific nuclease is or comprises a TALE Nuclease (Transcription Activator-Like Effector Nucleases (TALEN)). TALENs are restriction enzymes engineered to cut specific target DNA sequences. TALENs comprise a TAL effector (TALE) DNA-binding domain (which binds at or close to the target DNA), fused to a DNA cleavage domain which cuts target DNA. TALEs are engineered to bind to practically any desired DNA sequence. Thus in some embodiments, the TALEN comprises an N-terminal capping region, a DNA binding domain which may comprise at least one or more TALE monomers or half-monomers specifically ordered to target the genomic locus of interest, and a C-terminal capping region, wherein these three parts are arranged in a predetermined N-terminus to C-terminus orientation. Optionally, the TALEN includes at least one or more regulatory or functional protein domains.

In some embodiments, the TALE monomers or half monomers may be variant TALE monomers derived from natural or wild type TALE monomers but with altered amino acids at positions usually highly conserved in nature, and in particular have a combination of amino acids as RVDs that do not occur in nature, and which may recognize a nucleotide with a higher activity, specificity, and/or affinity than a naturally occurring RVD. The variants may include deletions, insertions and substitutions at the amino acid level, and transversions, transitions and inversions at the nucleic acid level at one or more locations. The variants may also include truncations.

In some embodiments, the TALE monomer/half monomer variants include homologous and functional derivatives of the parent molecules. In some embodiments, the variants are encoded by polynucleotides capable of hybridizing under high stringency conditions to the parent molecule-encoding wild-type nucleotide sequences.

In some embodiments, the DNA binding domain of the TALE has at least 5 of more TALE monomers and at least one or more half-monomers specifically ordered or arranged to target a genomic locus of interest. The construction and generation of TALEs or polypeptides of the disclosure may involve any of the methods known in the art.

Naturally occurring TALEs or “wild type TALEs” are nucleic acid binding proteins secreted by numerous species of proteobacteria. TALEs contain a nucleic acid binding domain composed of tandem repeats of highly conserved monomer polypeptides that are predominantly 33, 34 or 35 amino acids in length and that differ from each other mainly in amino acid positions 12 and 13. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X¹² and (*) indicates that X¹³ is absent. The DNA binding domain may comprise several repeats of TALE monomers and this may be represented as (X1-11-(X12X13)-X14-33 or 34 or 35)z, where z is optionally at least 5-40, such as 10-26.

The TALE monomers have a nucleotide binding affinity that is determined by the identity of the amino acids in its RVD. Polypeptide monomers with an RVD of NI preferentially bind to adenine (A), monomers with an RVD of NG preferentially bind to thymine (T), monomers with an RVD of HD preferentially bind to cytosine (C), monomers with an RVD of NN preferentially bind to both adenine (A) and guanine (G), monomers with an RVD of IG preferentially bind to T, monomers with an RVD of NS recognize all four base pairs and may bind to A, T, G or C. Thus, the number and order of the polypeptide monomer repeats in the nucleic acid binding domain of a TALE determines its nucleic acid target specificity. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In some embodiments, the TALE is a dTALE (or designerTALE), see Zhang et al., Nature Biotechnology 29:149-153 (2011), incorporated herein by reference.

In some embodiments, the TALE monomer comprises an RVD of HN or NH that preferentially binds to guanine, and the TALEs have high binding specificity for guanine containing target nucleic acid sequences. In come embodiments, polypeptide monomers having RVDs RN, NN, NK, SN, NH, KN, HN, NQ, HH, RG, KH, RH and SS preferentially bind to guanine. In some embodiments, polypeptide monomers having RVDs RN, NK, NQ, HH, KH, RH, SS and SN preferentially bind to guanine. In some embodiments, polypeptide monomers having RVDs HH, KH, NH, NK, NQ, RH, RN and SS preferentially bind to guanine. In some embodiments, the RVDs that have high binding specificity for guanine are RN, NH RH and KH. In some embodiments, polypeptide monomers having an RVD of NV preferentially bind to adenine and guanine as do monomers having the RVD IIN. Monomers having an RVD of NC preferentially bind to adenine, guanine and cytosine, and monomers having an RVD of S (or S*), bind to adenine, guanine, cytosine and thymine with comparable affinity. In more embodiments, monomers having RVDs of H*, HA, KA, N*, NA, NC, NS, RA, and S* bind to adenine, guanine, cytosine and thymine with comparable affinity. Such polypeptide monomers allow for the generation of degenerative TALEs able to bind to a repertoire of related, but not identical, target nucleic acid sequences.

In certain embodiments, the TALE polypeptide has a nucleic acid binding domain containing polypeptide monomers arranged in a predetermined N-terminus to C-terminus order such that each polypeptide monomer binds to a nucleotide of a predetermined target nucleic acid sequence, and where at least one of the polypeptide monomers has an RVD of HN or NH and preferentially binds to guanine, an RVD of NV and preferentially binds to adenine and guanine, an RVD of NC and preferentially binds to adenine, guanine and cytosine or an RVD of S and binds to adenine, guanine, cytosine and thymine.

In some embodiments, each polypeptide monomer of the nucleic acid binding domain that binds to adenine has an RVD of NI, NN, NV, NC or S.

In certain embodiments, each polypeptide monomer of the nucleic acid binding domain that binds to guanine has an RVD of HN, NH, NN, NV, NC or S.

In certain embodiments, each polypeptide monomer of the nucleic acid binding domain that binds to cytosine has an RVD of HD, NC or S.

In some embodiments, each polypeptide monomer that binds to thymine has an RVD of NG or S.

In some embodiments, each polypeptide monomer of the nucleic acid binding domain that binds to adenine has an RVD of NI.

In certain embodiments, each polypeptide monomer of the nucleic acid binding domain that binds to guanine has an RVD of IIN or NII.

In certain embodiments, each polypeptide monomer of the nucleic acid binding domain that binds to cytosine has an RVD of HD.

In some embodiments, each polypeptide monomer that binds to thymine has an RVD of NG.

In certain embodiments, the RVDs that have a specificity for adenine are NI, RI, KI, HI, and SI.

In certain embodiments, the RVDs that have a specificity for adenine are HN, SI and RI, most preferably the RVD for adenine specificity is SI.

In certain embodiments, the RVDs that have a specificity for thymine are NG, HG, RG and KG.

In certain embodiments, the RVDs that have a specificity for thymine are KG, HG and RG, most preferably the RVD for thymine specificity is KG or RG.

In certain embodiments, the RVDs that have a specificity for cytosine are HD, ND, KD, RD, HH, YG and SD.

In certain embodiments, the RVDs that have a specificity for cytosine are SD and RD.

FIG. 4B of WO 2012/067428 provides representative RVDs and the nucleotides they target, the entire content of which is hereby incorporated herein by reference.

In certain embodiments, the variant TALE monomers may comprise any of the RVDs that exhibit specificity for a nucleotide as depicted in FIG. 4A of WO2012/067428. All such TALE monomers allow for the generation of degenerative TALEs able to bind to a repertoire of related, but not identical, target nucleic acid sequences.

In certain embodiments, the RVD SH may have a specificity for G, the RVD IS may have a specificity for A, and the RVD IG may have a specificity for T.

In certain embodiments, the RVD NT may bind to G and A. In certain embodiments, the RVD NP may bind to A, T and C. In certain embodiments, at least one selected RVD may be NI, HD, NG, NN, KN, RN, NH, NQ, SS, SN, NK, KH, RH, HH, KI, HI, RI, SI, KG, HG, RG, SD, ND, KD, RD, YG, HN, NV, NS, HA, S*, N*, KA, H*, RA, NA or NC.

The predetermined N-terminal to C-terminal order of the one or more polypeptide monomers of the nucleic acid or DNA binding domain determines the corresponding predetermined target nucleic acid sequence to which the TALE or polypeptides of the disclosure may bind.

As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and polypeptides of the disclosure may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8 of WO 2012/067428). Therefore, it follows that the length of the nucleic acid or DNA being targeted is equal to the number of full monomers plus two (see FIG. 44 of WO 2012/067428).

In certain embodiments, nucleic acid binding domains are engineered to contain 5, 6, 7, 8, 9, 10, 11, 12, 13, 14, 15, 16, 17, 18, 19, 20, 21, 22, 23, 24, 25, or more polypeptide monomers arranged in a N-terminal to C-terminal direction to bind to a predetermined 5, 6, 7, 8, 9, 10, 11, 12, 13, 14, 15, 16, 17, 18, 19, 20, 21, 22, 23, 24, 25 nucleotide length nucleic acid sequence.

In certain embodiments, nucleic acid binding domains are engineered to contain 5, 6, 7, 8, 9, 10, 11, 12, 13, 14, 15, 16, 17, 18, 19, 20, 21, 22, 23, 24, 25, 26 or more full length polypeptide monomers that are specifically ordered or arranged to target nucleic acid sequences of length 7, 8, 9, 10, 11, 12, 13, 14, 15, 16, 17, 18, 19, 20, 21, 22, 23, 24, 25, 26, 27 and 28 nucleotides, respectively. In certain embodiments, the polypeptide monomers are contiguous. In some embodiments, half-monomers may be used in the place of one or more monomers, particularly if they are present at the C-terminus of the TALE.

Polypeptide monomers are generally 33, 34 or 35 amino acids in length. With the exception of the RVD, the amino acid sequences of polypeptide monomers are highly conserved or as described herein, the amino acids in a polypeptide monomer, with the exception of the RVD, exhibit patterns that effect TALE activity, the identification of which may be used in preferred embodiments of the disclosure.

In certain embodiments, when the DNA binding domain may comprise (X1-11-X12X13-X14-33 or 34 or 35)z, wherein X1-11 is a chain of 11 contiguous amino acids, wherein X12X13 is a repeat variable di-residue (RVD), wherein X14-33 or 34 or 35 is a chain of 21, 22 or 23 contiguous amino acids, wherein z is at least 5 to 26, then the preferred combinations of amino acids are LTLD or LTLA or LTQV at X1-4, or EQHG or RDHG at positions X30-33 or X31-34 or X32-35. Furthermore, other amino acid combinations of interest in the monomers are LTPD at X1-4 and NQALE at XI 6-20 and DHG at X32-34 when the monomer is 34 amino acids in length. When the monomer is 33 or 35 amino acids long, then the corresponding shift occurs in the positions of the contiguous amino acids NQALE and DHG. In certain embodiments, NQALE is at X15-19 or X17-21 and DHG is at X31-33 or X33-35.

In certain embodiments, amino acid combinations of interest in the monomers, are LTPD at X1-4 and KRALE at X16-20 and AHG at X32-34 or LTPE at X1-4 and KRALE at XI 6-20 and DHG at X32-34 when the monomer is 34 amino acids in length. When the monomer is 33 or 35 amino acids long, the corresponding shift occurs in the positions of the contiguous amino acids KRALE, AHG and DHG. In certain embodiments, the positions of the contiguous amino acids may be (LTPD at X1-4 and KRALE at X15-19 and AHG at X31-33) or (LTPE at X1-4 and KRALE at X15-19 and DHG at X31-33) or (LTPD at X1-4 and KRALE at X17-21 and AHG at X33-35) or (LTPE at X1-4 and KRALE at X17-21 and DIIG at X33-35).

In certain embodiments, contiguous amino acids [NGKQALE] are present at positions X14-20 or X13-19 or X15-21. These representative positions put forward various embodiments of the disclosure and provide guidance to identify additional amino acids of interest or combinations of amino acids of interest in all the TALE monomers (see FIGS. 24A-24F, and 25 of WO 2012/067428).

A further listing of TALE monomers excluding the RVDs which may be denoted in a sequence (X1-11-X14-34 or XI-11-X14-35), wherein X is any amino acid and the subscript is the amino acid position is provided in FIG. 24A-F of WO 2012/067428, which is incorporated herein by reference.

In certain embodiments, TALE polypeptide binding efficiency is increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the disclosure.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE (including TALEs) polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. In certain embodiments, C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result. % homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Additional sequences for the conserved portions of polypeptide monomers and for N-terminal and C-terminal capping regions are included in the sequences with the following gene accession numbers: AAW59491.1, AAQ797732, YP_450163.1, YP_001912778.1, ZP_02242672.1, AAW59493.1, AAY54170.1, ZP_02245314.1, ZP_02243372.1, AAT46123.1, AAW59492.1, YP_451030.1, YP_001915105.1, ZP_02242534.1, AAW77510.1, ACD11364.1, ZP_02245056.1, ZP_02245055.1, ZP_02242539.1, ZP_02241531.1, ZP_02243779.1, AAN01357.1, ZP_02245177.1, ZP_02243366.1, ZP_02241530.1, AAS58130.3, ZP_02242537.1, YP_200918.1, YP_200770.1, YP_451187.1, YP_451156.1, AAS58127.2, YP_451027.1, UR_451025.1, AAA92974.1, UR_001913755.1, ABB70183.1, UR_451893.1, UR_450167.1, ABY60855.1, UR_200767.1, ZR_02245186.1, ZR_02242931.1, ZR_02242535.1, AAU54169.1, UR_450165.1, UR_001913452.1, AAS58129.3, ACM44927.1, ZR_02244836.1, AAT46125.1, UR_450161.1, ZR_02242546.1, AAT46122.1, UR_451897.1, AAF98343.1, UR_001913484.1, AAY54166.1, UR_001915093.1, UR_001913457.1, ZR_02242538.1, UR_200766.1, UR_453043.1, UR_001915089.1, UR_001912981.1, ZR_02242929.1, UR_001911730.1, UR_201654.1, UR_199877.1, ABB70129.1, UR_451696.1, UR_199876.1, AAS75145.1, AAT46124.1, UR_200914.1, UR 001915101.1, ZR_02242540.1, AAG02079.2, UR_451895.1, YP 451189.1, UR_200915.1, AAS46027.1, UR_001913759.1, UR_001912987.1, AAS58128.2, AAS46026.1, UR_201653.1, UR_202894.1, UR_001913480.1, ZR_02242666.1, R_001912775.1, ZR_02242662.1, AAS46025.1, AAC43587.1, BAA37119.1, NPJ544725.1, AB077779.1, BAA37120.1, ACZ62652.1, BAF46271.1, ACZ62653.1, NPJ544793.1, AB077780.1, ZR_02243740.1, ZR_02242930.1, AAB69865.1, AAY54168.1, ZR_02245191.1, UR_001915097.1, ZR_02241539.1, UR_451158.1, BAA37121.1, UR_001913182.1, UR_200903.1, ZR_02242528.1, ZR_06705357.1, ZR_06706392.1, AD148328.1, ZR_06731493.1, ADI48327.1, AB077782.1, ZR 06731656.1, NR_942641.1, AAY43360.1, ZR_06730254.1, ACN39605.1, UR_451894.1, UR_201652.1, UR_001965982.1, BAF46269.1, NPJ544708.1, ACN82432.1, AB077781.1, P14727.2, BAF46272.1, AAY43359.1, BAF46270.1, NR_644743.1, ABG37631.1, AAB00675.1, YP 199878.1, ZR_02242536.1, CAA48680.1, ADM80412.1, AAA27592.1, ABG37632.1. ABP97430.1, ZR_06733167.1, AAY43358.1, 2KQ5_A, BAD42396.1, AB027075.1, UR_002253357.1, UR_002252977.1, AB027074.1, AB027067.1, AB027072.1, AB027068.1, UR_003750492.1, AB027073.1, NR_519936.1, AB027071.1, AB027070.1, and AB027069.1, each of which is hereby incorporated by reference.

In some embodiments, the TALEs described herein also include a nuclear localization signal and/or cellular uptake signal. Such signals are known in the art and may target a TALE to the nucleus and/or intracellular compartment of a cell. Such cellular uptake signals include, but are not limited to, the minimal Tat protein transduction domain which spans residues 47-57 of the human immunodeficiency virus Tat protein.

In some embodiments, the TALEs described herein include a nucleic acid or DNA binding domain that is a non-TALE nucleic acid or a non-TALE DNA binding domain.

As used herein the term “non-TALE DNA binding domain” refers to a DNA binding domain that has a nucleic acid sequence corresponding to a nucleic acid sequence which is not substantially homologous to a nucleic acid that encodes for a TALE protein or fragment thereof, e.g., a nucleic acid sequence which is different from a nucleic acid that encodes for a TALE protein and which is derived from the same or a different organism.

In certain embodiments, the TALEs described herein include a nucleic acid or DNA binding domain that is linked to a non-TALE polypeptide.

A “non-TALE polypeptide” refers to a polypeptide having an amino acid sequence corresponding to a protein which is not substantially homologous to a TALE protein or fragment thereof, e.g., a protein which is different from a TALE protein and which is derived from the same or a different organism. In this context, the term “linked” is intended include any manner by which the nucleic acid binding domain and the non-TALE polypeptide could be connected to each other, including, for example, through peptide bonds by being part of the same polypeptide chain or through other covalent interactions, such as a chemical linker. The non-TALE polypeptide may be linked, for example to the N-terminus and/or C-terminus of the nucleic acid binding domain, may be linked to a C-terminal or N-terminal cap region, or may be connected to the nucleic acid binding domain indirectly.

In certain embodiments, the TALEs or polypeptides of the disclosure comprise chimeric DNA binding domains. Chimeric DNA binding domains may be generated by fusing a full TALE (including the N- and C-terminal capping regions) with another TALE or non-TALE DNA binding domain such as zinc finger (ZF), helix-loop-helix, or catalytically-inactivated DNA endonucleases (e.g., EcoRI, meganucleases, etc.), or parts of TALE may be fused to other DNA binding domains. The chimeric domain may have novel DNA binding specificity that combines the specificity of both domains.

In certain embodiments, the TALE polypeptides of the disclosure include a nucleic acid binding domain linked to the one or more effector domains. In certain embodiments, the effector domain is a nickase or nuclease.

### ZFNs

In certain embodiments, the sequence-specific nuclease is a zine finger nuclease (ZFN), such as an artificial zinc-finger nuclease having arrays of zine-finger (ZF) modules to target new DNA-binding sites in a target sequence (e.g., target sequence or target site in the genome). Each zine finger module in a ZF array targets three DNA bases. A customized array of individual zinc finger domains is assembled into a ZF protein (ZFP). The resulting ZFP can be linked to a functional domain such as a nuclease.

ZF nucleases (ZFN) may be used as alternative programmable nucleases for use in retron-based editing in place of RNA-guide nucleases. ZFN proteins have been extensively described in the art, for example, in Carroll et al., “Genome Engineering with Zinc-Finger Nucleases,” Genetics, August 2011, Vol. 188: 773-782; Durai et al., “Zinc finger nucleases: custom-designed molecular scissors for genome engineering of plant and mammalian cells,” Nucleic Acids Res, 2005, Vol. 33: 5978-90; and Gaj et al., “ZFN, TALEN, and CRISPR/Cas-based methods for genome engineering,” Trends Biotechnol. 2013, Vol. 31: 397-405, each of which are incorporated herein by reference in their entireties.

In certain embodiments, the ZF-linked nuclease is a catalytic domain of the Type IIS restriction enzyme FokI (see Kim et al., PNAS U.S.A. 91:883-887, 1994; Kim et al., PNAS U.S.A. 93:1156-1160, 1996, both incorporated herein by reference).

In certain embodiments, the ZFN comprises paired ZFN heterodimers, resulting in increased cleavage specificity and/or decreased off-target activity. In this embodiment, each ZFN in the heterodimer targets different nucleotide sequences separated by a short spacer (see Doyon et al., Nat. Methods 8:74-79, 2011, incorporated herein by reference).

In certain embodiments, the ZFN comprises a polynucleotide-binding domain (comprising multiple sequence-specific ZF modules) and a polynucleotide cleavage nickase domain.

In certain embodiments, the ZFs are engineered using libraries of two finger modules.

In certain embodiments, strings of two-finger units are used in ZFNs to improve DNA binding specificity from polyzine finger peptides (see PNAS USA 98: 1437-1441, incorporated herein by reference).

In certain embodiments, the ZFN has more than 3 fingers. In certain embodiments, the ZFN has 4, 5, or 6 fingers. In certain embodiments, the ZF modules in the ZFN are separated by one or more linkers to improve specificity.

In certain embodiments, the ZF of the ZFN includes substitutions in the dimer interface of the cleavage domain that prevent homodimerization between ZFs, but allow heterodimers to form.

In certain embodiments, the ZF of the ZFN has a design that retains activity while suppressing homodimerization.

In certain embodiments, the ZFN is any one of the ZF nucleases in Table 1 of Carroll et al., Genetics 188(4):773-782, 2011, incorporated herein by reference.

General principles and guidance for generating ZF, ZF arrays, and ZFN can be found in the art, such as the modular design (where the different modules can be rearranged and assembled into new combinations for new targets) of the ZF or ZF arrays in the ZFN as taught in Carroll et al., Nat.

Protoc. 1: 1329-1341, 2006 (incorporated herein by reference); the new three-finger sets for engineered ZFs generated by using partially randomized libraries; profiling the DNA-binding specificities of engineered Cys2His2 zinc finger domains using a rapid cell-based method (see Nucleic Acids Res. 35: e81, incorporated by reference). ZFs for certain DNA triplets that work well in neighbor combination are described in Sander et al., 2011. Selection-free zine-finger-nuclease engineering by context-dependent assembly (CoDA) is taught in Nat. Methods 8: 67-69). ToolGen describes the individual fingers in their collection that are best behaved in modular assembly (Kim et al., 2011). Preassembled zinc-finger arrays for rapid construction of ZFNs are taught in Nat. Methods 8:7.

Additional, non-limiting ZFs and AFNz that can be adapted for use in the instant disclosure include those described in WO2010/065123, WO2000/041566, WO2003/080809, WO2015/143046, WO2016/183298, WO2013/044008, WO2015/031619, WO2017/136049, WO2016/014794, WO2017/091512, WO1995/009233, WO2000/023464, WO2000/042219, WO2002/026960, WO2001/083793; U.S. Pat. Nos. 9,428,756, 9,145,565, 8,846,578, 8,524,874, 6,777,185, 6,599,692, 7,235,354, 6,503,717, 7,491,531, 7,943,553, 7,262,054, 8,680,021, 7,705,139, 7,273,923, 6,780,590, 6,785,613, 7,788,044, 7,177,766, 6,453,242, 6,794,136, 7,358,085, 8,383,766, 7,030,215, 7,013,219, 7,361,635, 7,939,327, 8,772,453, 9,163,245, 7,045,304, 8,313,925, 9,260,726, 6,689,558, 8,466,267, 7,253,273, 7,947,873, 9,388,426, 8,153,399, 8,569,253, 8,524,221, 7,951,925, 9,115,409, 8,772,008, 9,121,072, 9,624,498, 6,979,539, 9,491,934, 6,933,113, 9,567,609, 7,070,934, 9,624,509, 8,735,153, 9,567,573, 6,919,204, US2002-0081614, US2004-0203064, US2006-0166263, US2006-0292621, US2003-0134318, US2006-0294617, US2007-0287189, US2007-0065931, US2003-0105593, US2003-0108880, US2009-0305402, US2008-0209587, US2013-0123484, US2004-0091991, US2009-0305977, US2008-0233641, US2014-0287500, US2011-0287512, US2009-0258363, US2013-0244332, US2007-0134796, US2010-0256221, US2005-0267061, US2012-0204282, US2012-0252122, US2010-0311124, US2016-0215298, US2008-0031109, US2014-0017214, US2015-0267205, US2004-0235002, US2004-0204345, US2015-0064789, US2006-0063231, US2011-0265198, US2017-0218349, all incorporated herein by reference.

Polynucleotides and vectors capable of expressing one or more of the ZFNs are also provided herein, which can be part of the vector system of the disclosure. The polynucleotides and vectors can be expressed in a cell, such as a eukaryotic cell, a mammalian cell, or a human cell. Suitable vectors, cells and expression systems are described in greater detail elsewhere herein, and can be suitable for use with the TALEs, the meganucleases, and the CRISPR-Cas nucleases.

### Meganucleases

In some embodiments, the gene editing system comprises meganucleases. Meganucleases are homing endonucleases discovered in yeast that recognize fairly long DNA sequences, and create double-strand breaks that are mended via stimulation of homologous recombination. Meganucleases are sequence-specific endonucleases that use large (recognition sites to generate accurate double-strand breaks (DSBs), promoting efficient gene targeting through homologous recombination (HR). Meganuclease enzymes and editing systems comprising meganucleases have been described in the literature, including the following references, each of which are incorporated herein in their entireties by reference. Khalil AM. The genome editing revolution: review. J Genet Eng Biotechnol. 2020 Oct. 29; 18(1):68. doi: 10.1186/s43141-020-00078-y. PMID: 33123803; PMCID: PMC7596157. Lanigan T M, Kopera H C, Saunders T L. Principles of Genetic Engineering. Genes (Basel). 2020 Mar. 10; 11(3):291. doi: 10.3390/genes11030291. PMID: 32164255; PMCID: PMC7140808. Arnould S, Delenda C, Grizot S, Desseaux C, Piques F, Silva G H, Smith J. The I-CreI meganuclease and its engineered derivatives: applications from cell modification to gene therapy. Protein Eng Des Sel. 2011 January; 24(1-2):27-31. doi: 10.1093/protein/gzq083. Epub 2010 Nov. 3. PMID: 21047873. Piques F, Duchateau P. Meganucleases and DNA double-strand break-induced recombination: perspectives for gene therapy. Curr Gene Ther. 2007 February; 7(1):49-66. doi: 10.2174/156652307779940216. PMID: 17305528. Zekonyte U, Bacman S R, Smith J, Shoop W, Pereira C V, Tomberlin G, Stewart J, Jantz D, Moraes C T. Mitochondrial targeted meganuclease as a platform to eliminate mutant mtDNA in vivo. Nat Commun. 2021 May 28; 12(1):3210. doi: 10.1038/s41467-021-23561-7. PMID: 34050192; PMCID: PMC8163834.

Meganuclease enzymes and editing systems comprising meganucleases have also been described in the patent literature, including the following references, each of which are incorporated herein in their entireties by reference.

| Pub Number | Title |
|---|---|
| WO2022226316A1 | COMPOSITIONS AND METHODS FOR GENERATING MALE STERILE |
| | PLANTS |
| WO2022226305A1 | ENGINEERED MEGANUCLEASES THAT TARGET HUMAN |
| | MITOCHONDRIAL GENOMES |
| WO2022226303A1 | ENGINEERED MEGANUCLEASES THAT TARGET HUMAN |
| | MITOCHONDRIAL GENOMES |
| WO2022165111A1 | MODULATION OF TGF BETA SIGNALING IN GENETICALLY-MODIFIED |
| | EUKARYOTIC CELLS |
| WO2022104062A1 | ENGINEERED MEGANUCLEASES HAVING SPECIFICITY FOR |
| | RECOGNITION SEQUENCES IN THE DYSTROPHIN GENE |
| WO2022076547A1 | LIPID NANOPARTICLE COMPOSITIONS |
| WO2022040582A1 | ENGINEERED MEGANUCLEASES HAVING SPECIFICITY FOR A |
| | RECOGNITION SEQUENCE IN THE TRANSTHYRETIN GENE |
| WO2022035793A1 | ANTIBODIES AND FRAGMENTS SPECIFIC FOR B-CELL MATURATION |
| | ANTIGEN AND USES THEREOF |
| WO2021231959A1 | METHODS FOR IMMUNOTHERAPY |
| WO2021231954A1 | METHODS FOR IMMUNOTHERAPY |
| WO2021231495A1 | TREATMENT OF RETINITIS PIGMENTOSA USING IMPROVED |
| | ENGINEERED MEGANUCLEASES |
| WO2021231259A1 | SELF-LIMITING VIRAL VECTORS ENCODING NUCLEASES |
| WO2021158915A1 | RECOMBINANT ADENO-ASSOCIATED VIRUS COMPOSITIONS AND |
| | METHODS FOR PRODUCING AND USING THE SAME |
| WO2021113765A1 | OPTIMIZED ENGINEERED MEGANUCLEASES HAVING SPECIFICITY |
| | FOR A RECOGNITION SEQUENCE IN THE HEPATITIS B VIRUS |
| | GENOME |
| WO2021113543A1 | METHODS FOR CANCER IMMUNOTHERAPY, USING |
| | LYMPHODEPLETION REGIMENS AND CD19, CD20 OR BCMA |
| | ALLOGENEIC CAR T CELLS |
| WO2021087305A1 | CD20 CHIMERIC ANTIGEN RECEPTORS AND METHODS OF USE FOR |
| | IMMUNOTHERAPY |
| WO2021035170A1 | COMPOSITIONS AND METHODS FOR TCR REPROGRAMMING USING |
| | FUSION PROTEINS |
| WO2021035054A1 | LYMPHODEPLETION DOSING REGIMENS FOR CELLULAR |
| | IMMUNOTHERAPIES |
| WO2021016608A1 | COMPOSITIONS AND METHODS FOR SEQUENTIAL STACKING OF |
| | NUCLEIC ACID SEQUENCES INTO A GENOMIC LOCUS |
| WO2020227534A1 | OPTIMIZATION OF ENGINEERED MEGANUCLEASES FOR |
| | RECOGNITION SEQUENCES |
| WO2020206248A1 | GENETICALLY-MODIFIED IMMUNE CELLS COMPRISING A |
| | MICRORNA-ADAPTED SHRNA (SHRNAMIR) |
| WO2020206231A1 | METHODS OF PREPARING POPULATIONS OF GENETICALLY- |
| | MODIFIED IMMUNE CELLS |
| WO2020146807A1 | GENETICALLY-MODIFIED CELLS COMPRISING A MODIFIED |
| | TRANSFERRIN GENE |
| WO2020132659A1 | GENETIC MODIFICATION OF THE HYDROXYACID OXIDASE 1 GENE |
| | FOR TREATMENT OF PRIMARY HYPEROXALURIA |
| WO2019200247A1 | OPTIMIZED ENGINEERED MEGANUCLEASES HAVING SPECIFICITY |
| | FOR A RECOGNITION SEQUENCE IN THE HEPATITIS B VIRUS |
| | GENOME |
| WO2019200122A1 | OPTIMIZED ENGINEERED NUCLEASES HAVING SPECIFICITY FOR |
| | THE HUMAN T CELL RECEPTOR ALPHA CONSTANT REGION GENE |
| WO2019089913A1 | ENGINEERED NUCLEASES THAT TARGET HUMAN AND CANINE |
| | FACTOR VIII GENES AS A TREATMENT FOR HEMOPHILIA A |
| WO2019070856A1 | MODIFIED EPIDERMAL GROWTH FACTOR RECEPTOR PEPTIDES FOR |
| | USE IN GENETICALLY-MODIFIED CELLS |
| WO2019005957A1 | GENETICALLY-MODIFIED T CELLS COMPRISING A MODIFIED |
| | INTRON IN THE T CELL RECEPTOR ALPHA GENE |
| WO2018208837A1 | NUCLEIC ACID MOLECULES ENCODING AN ENGINEERED ANTIGEN |
| | RECEPTOR AND AN INHIBITORY NUCLEIC ACID MOLECULE AND |
| | METHODS OF USE THEREOF |
| WO2018201144A1 | METHODS FOR REDUCING DNA-INDUCED CYTOTOXICITY AND |
| | ENHANCING GENE EDITING IN PRIMARY CELLS |
| WO2018195449A1 | ENGINEERED MEGANUCLEASES SPECIFIC FOR RECOGNITION |
| | SEQUENCES IN THE PCSK9 GENE |
| WO2018071849A2 | ENGINEERED MEGANUCLEASES SPECIFIC FOR RECOGNITION |
| | SEQUENCES IN THE HEPATITIS B VIRUS GENOME |
| WO2018067697A1 | CO-STIMULATORY DOMAINS FOR USE IN GENETICALLY-MODIFIED |
| | CELLS |
| WO2017192741A1 | ENGINEERED NUCLEASES USEFUL FOR TREATMENT OF |
| | HEMOPHILIA A |
| WO2017112859A1 | ENGINEERED MEGANUCLEASES WITH RECOGNITION SEQUENCES |
| | FOUND IN THE HUMAN BETA-2 MICROGLOBULIN GENE |
| WO2017062451A1 | GENETICALLY-MODIFIED CELLS COMPRISING A MODIFIED HUMAN |
| | T CELL RECEPTOR ALPHA CONSTANT REGION GENE |
| WO2017062439A1 | ENGINEERED MEGANUCLEASES WITH RECOGNITION SEQUENCES |
| | FOUND IN THE HUMAN T CELL RECEPTOR ALPHA CONSTANT |
| | REGION GENE |
| WO2017044649A1 | TREATMENT OF RETINITIS PIGMENTOSA USING ENGINEERED |
| | MEGANUCLEASES |
| WO2016205825A1 | SELF-LIMITING VIRAL VECTORS ENCODING NUCLEASES |
| WO2016179112A1 | PRECISE DELETION OF CHROMOSCOMAL SEQUENCES IN VIVO AND |
| | TREATMENT OF NUCLEOTIDE REPEAT EXPANSION DISORDERS |
| | USING ENGINEERED NUCLEASES |
| WO2015138739A2 | DYSTROPHIN GENE OXON DELETION USING ENGINEERED |
| | NUCLEASES |
| WO2012167192A2 | METHODS AND PRODUCTS FOR PRODUCING ENGINEERED |
| | MAMMALIAN CELL LINES WITH AMPLIFIED TRANSGENES |
| WO2010009147A1 | RECOGNITION SEQUENCES FOR I-CREI-DERIVED MEGANUCLEASES |
| | AND USES THEREOF |
| WO2009134714A2 | FUSION MOLECULES OF RATIONALLY-DESIGNED DNA-BINDING |
| | PROTEINS AND EFFECTOR DOMAINS |
| WO2009114321A2 | RATIONALLY-DESIGNED MEGANUCLEASES FOR MAIZE GENOME |
| | ENGINEERING |
| WO2009076292A2 | RATIONALLY-DESIGNED MEGANUCLEASES WITH RECOGNITION |
| | SEQUENCES FOUND IN DNASE HYPERSENSITIVE REGIONS OF THE |
| | HUMAN GENOME |
| WO2009059195A2 | RATIONALLY-DESIGNED SINGLE-CHAIN MEGANUCLEASES WITH |
| | NON-PALINDROMIC RECOGNITION SEQUENCES |
| WO2007047859A2 | RATIONALLY-DESIGNED MEGANUCLEASES WITH ALTERED |
| | SEQUENCE SPECIFICITY AND DNA-BINDING AFFINITY |
| US20130340121A1 | Temperature-Dependent Meganuclease Activity |

In other aspects, the gene editing systems described herein may comprise one or more additional accessory proteins having genome modifying functions, including recombinases, invertases, nucleases, polymerases (e.g., reverse transcriptases), ligases, deaminases, transposases, or DNA binding domains. In various embodiments, the accessory proteins may be provided separately. In other embodiments, the accessory proteins may be fused to another component of a given gene editing system, such as a CRISPR-Cas9, through a linker.

### Guide RNA Components

### Guide RNAs

The present disclosure further provides guide RNAs for use in accordance with the disclosed nucleic acid programmable DNA binding proteins (e.g., Cas9) for use in methods of editing. The disclosure provides guide RNAs that are designed to recognize target sequences. Such gRNAs may be designed to have guide sequences (or “spacers”) having complementarity to a target sequence. Such gRNAs may be designed to have not only a guide sequences having complementarity to a target sequence to be edited, but also to have a backbone sequence that interacts specifically with the nucleic acid programmable DNA binding protein.

In some embodiments, the guide RNA may be 15-100 nucleotides in length and comprise a sequence of at least 10, at least 15, or at least 20 contiguous nucleotides that is complementary to a target nucleotide sequence. The guide RNA may comprise a spacer sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target nucleotide sequence. In some cases, the guide sequence has a length in a range of from 17-30 nucleotides (nt) (e.g., from 17-25, 17-22, 17-20, 19-30, 19-25, 19-22, 19-20, 20-30, 20-25, or 20-22 nt). In some cases, the guide sequence has a length in a range of from 17-25 nucleotides (nt) (e.g., from 17-22, 17-20, 19-25, 19-22, 19-20, 20-25, or 20-22 nt). In some cases, the guide sequence has a length of 17 or more nt (e.g., 18 or more, 19 or more, 20 or more, 21 or more, or 22 or more nt; 19 nt, 20 nt, 21 nt, 22 nt, 23 nt, 24 nt, 25 nt, etc.). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

In some cases, the spacer sequence has a length of from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt).

A subject guide RNA can interact with a target nucleic acid (e.g., double stranded DNA (dsDNA), single stranded DNA (ssDNA), single stranded RNA (ssRNA), or double stranded RNA (dsRNA)) in a sequence-specific manner via hybridization (i.e., base pairing).

The guide RNA can be modified to hybridize to any desired target sequence (e.g., while taking the PAM into account, e.g., when targeting a dsDNA target) within a target nucleic acid (e.g., a eukaryotic target nucleic acid such as genomic DNA). In some cases, the percent complementarity between the spacer sequence of the guide and the target site of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the spacer and the target site of the target nucleic acid is 80% or more (e.g., 85% or more, 90% or more, 95% or more, 97% or more, 98% or more, 99% or more, or 100%). In some cases, the percent complementarity between the spacer and the target site of the target nucleic acid is 90% or more (e.g., 95% or more, 97% or more, 98% or more, 99% or more, or 100%). In some cases, the percent complementarity between the spacer and the target site of the target nucleic acid is 100%.

In some cases, the percent complementarity between the spacer sequence and the target site of the target nucleic acid is 100% over an at least 5-nucleotide contiguous region of the spacer. In some cases, the percent complementarity between the guide sequence and the target site of the target nucleic acid over an at least 6-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 7-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 8-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 9-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 10-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 11-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 12-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 13-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 14-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 15-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 16-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 17-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 18-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 19-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 20-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 21-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 22-nucleotide contiguous region of the spacer is 60% or more (e.g., 70% or more, 75% or more, 80% or more, 85% or more, 90% or more, 95% or more, 97% or more, 98% or more, 99% or more, or 100%).

In some cases, the percent complementarity between the spacer sequence and the target site of the target nucleic acid is 100% over an at least 5-10 nucleotide contiguous region of the spacer. In some cases, the percent complementarity between the guide sequence and the target site of the target nucleic acid over an at least 6-11 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 7-12 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 8-13 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 9-14 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 10-15 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 11-16 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 12-17 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 13-18 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 14-19 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 15-20 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 16-21 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 17-22 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 18-23 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 19-24 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 20-25 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 21-26 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 22-27 nucleotide contiguous region of the spacer is 60% or more (e.g., 70% or more, 75% or more, 80% or more, 85% or more, 90% or more, 95% or more, 97% or more, 98% or more, 99% or more, or 100%).

In various embodiments, the guide RNAs may have a scaffold or core region that complexes with a cognate nucleic acid programmable DNA binding protein (e.g., CRISPR Cas9 or Cas12a). In some cases, a guide scaffold can have two stretches of nucleotides that are complementary to one another and hybridize to form a double stranded RNA duplex (dsRNA duplex). Thus, in some cases, the protein binding segment of a guide RNA includes a dsRNA duplex. In some embodiments, the dsRNA duplex region includes a range of from 5-25 base pairs (bp) (e.g., from 5-22, 5-20, 5-18, 5-15, 5-12, 5-10, 5-8, 8-25, 8-22, 8-18, 8-15, 8-12, 12-25, 12-22, 12-18, 12-15, 13-25, 13-22, 13-18, 13-15, 14-25, 14-22, 14-18, 14-15, 15-25, 15-22, 15-18, 17-25, 17-22, or 17-18 bp, e.g., 5 bp, 6 bp, 7 bp, 8 bp, 9 bp, 10 bp, etc.). In some cases, the dsRNA duplex region includes a range of from 6-15 base pairs (bp) (e.g., from 6-12, 6-10, or 6-8 bp, e.g., 6 bp, 7 bp, 8 bp, 9 bp, 10 bp, etc.). In some cases, the duplex region includes 5 or more bp (e.g., 6 or more, 7 or more, or 8 or more bp). In some cases, the duplex region includes 6 or more bp (e.g., 7 or more, or 8 or more bp). In some cases, not all nucleotides of the duplex region are paired, and therefore the duplex forming region can include a bulge. The term “bulge” herein is used to mean a stretch of nucleotides (which can be one nucleotide) that do not contribute to a double stranded duplex, but which are surround 5′ and 3′ by nucleotides that do contribute, and as such a bulge is considered part of the duplex region. In some cases, the dsRNA includes 1 or more bulges (e.g., 2 or more, 3 or more, 4 or more bulges). In some cases, the dsRNA duplex includes 2 or more bulges (e.g., 3 or more, 4 or more bulges). In some cases, the dsRNA duplex includes 1-5 bulges (e.g., 1-4, 1-3, 2-5, 2-4, or 2-3 bulges).

Thus, in some cases, the stretches of nucleotides that hybridize to one another to form the dsRNA duplex in a guide scaffold region have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 85%-100% complementarity (e.g., 90%-100%, 95%-100% complementarity) with one another. In some cases, the stretches of nucleotides that hybridize to one another to form the dsRNA duplex have 70%-95% complementarity (e.g., 75%-95%, 80%-95%, 85%-95%, 90%-95% complementarity) with one another. In other words, in some cases, the dsRNA duplex includes two stretches of nucleotides that have 70%-100% complementarity (e.g., 75%-100%, 80%-10%, 85%-100%, 90%-100%, 95%-100% complementarity) with one another. In some cases, the dsRNA duplex includes two stretches of nucleotides that have 85%-100% complementarity (e.g., 90%-100%, 95%-100% complementarity) with one another. In some cases, the dsRNA duplex includes two stretches of nucleotides that have 70%-95% complementarity (e.g., 75%-95%, 80%-95%, 85%-95%, 90%-95% complementarity) with one another.

In various embodiments, the scaffold region of a guide RNA can also include one or more (1, 2, 3, 4, 5, etc.) mutations relative to a naturally occurring scaffold region. For example, in some cases a base pair can be maintained while the nucleotides contributing to the base pair from each segment can be different. In some cases, the duplex region of a subject guide RNA includes more paired bases, less paired bases, a smaller bulge, a larger bulge, fewer bulges, more bulges, or any convenient combination thereof, as compared to a naturally occurring duplex region (of a naturally occurring guide RNA).

Examples of various guide RNAs can be found in the art, and in some cases variations similar to those introduced into Cas9 guide RNAs can also be introduced into guide RNAs of the present disclosure (e.g., mutations to the dsRNA duplex region, extension of the 5′ or 3′ end for added stability for to provide for interaction with another protein, and the like). For example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5): 1173-83; Wang et al., Cell. 2013 May 9; 153(4):910-8; Auer et al., Genome Res. 2013 Oct. 31; Chen et al., Nucleic Acids Res. 2013 Nov. 1 ; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et. al, Nucleic Acids Res. 2013 Nov. 1; 41(20):e187; Hu et al., Cell Res. 2013 November; 23(ll):1322-5; Jiang et al., Nucleic Acids Res. 2013 Nov. 1; 41(20):e188; Larson et al., Nat Protoc. 2013 November; 8(11):2180-96; Mali et. at., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

### Guide RNA Modifications

In one embodiment, the guide RNAs (including pegRNAs) contemplated herein comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a guide RNA (including pegRNA) component nucleic acid comprises ribonucleotides and non-ribonucleotides. In one such embodiment, a guide RNA (including pegRNA) component comprises one or more ribonucleotides and one or more deoxyribonucleotides. In an embodiment of the disclosure, the guide RNA (including pegRNA) component comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA).

Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of coRNA chemical modifications include, without limitation, incorporation of 2′-O-methyl (M), 2′-O-methyl 3′ phosphorothioate (MS), S-constrained ethyl(cEt), or 2′-O-methyl 3′thioPACE (MSP) at one or more terminal nucleotides. Such chemically modified oRNA components can comprise increased stability and increased activity as compared to unmodified oRNA components, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112: 11870-11875; Sharma et al., MedChemComm., 2014, 5: 1454-1471; Hendel et al., Nat. Biotechnol. (2015) 33(9): 985-989; Li et al., Nature Biomedical Engineering, 2017, 1, 0066 D01: 10.1038/s41551-017-0066). In one embodiment, the 5′ and/or 3′ end of a guide RNA (including pegRNA) component is modified by a variety of functional moieties including fluorescent dyes, polyethylene glycol, cholesterol, proteins, or detection tags. (See Kelly et al., 2016, J. Biotech. 233:74-83). In one embodiment, a guide RNA (including pegRNA) component comprises ribonucleotides in a region that binds to a target sequence and one or more deoxyribonucletides and/or nucleotide analogs in a region that binds to a nucleic acid programmable DNA binding protein (e.g., Cas9 nickase).

In an embodiment, deoxyribonucleotides and/or nucleotide analogs are incorporated in engineered guide RNA (including pegRNA) component structures. In one embodiment, 3-5 nucleotides at either the 3′ or the 5′ end of a guide RNA (including pegRNA) component is chemically modified. In one embodiment, only minor modifications are introduced in the seed region, such as 2′-F modifications. In one embodiment, 2′-F modification is introduced at the 3′ end of a guide RNA (including pegRNA) component. In one embodiment, three to five nucleotides at the 5′ and/or the 3′ end of the reRNA component are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In one embodiment, all of the phosphodiester bonds of a guide RNA (including pegRNA) component are substituted with phosphorothioates (PS) for enhancing levels of gene disruption. In one embodiment, more than five nucleotides at the 5′ and/or the 3′ end of the guide RNA (including pegRNA) component are chemically modified with 2′-O-Me, 2′-F or S-constrained ethyl(cEt). Such chemically modified guide RNA (including pegRNA) component can mediate enhanced levels of gene disruption (see Ragdarm et al., 0215, PNAS, E7110-E7111). In an embodiment of the disclosure, a guide RNA (including pegRNA) component is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide RNA (including pegRNA) component by a linker, such as an alkyl chain. In one embodiment, the chemical moiety of the modified nucleic acid component can be used to attach the guide RNA (including pegRNA) component to another molecule, such as DNA, RNA, protein, or nanoparticles. Such chemically modified guide RNA (including pegRNA) component can be used to identify or enrich cells generically edited by a gene editing system described herein.

Other guide RNA (including pegRNA) modifications are described in Kim, D. Y., Lee, J. M., Moon, S. B. et al. Efficient CRISPR editing with a hypercompact Cas12f1 and engineered guide RNAs delivered by adeno-associated virus. Nat Biotechnol 40, 94-102 (2022).

Accordingly, in various aspects of the disclosure, the guide RNA (including pegRNA) are modified in one or more locations within the molecule. MS1, an internal penta(uridinylate) (UUUUU) sequence in the tracrRNA; MS2, the 3′ terminus of the crRNA; MS3, the ‘stem 1’ region of the tracrRNA; MS4, the tracrRNA-crRNA complementary region; and MS5, the ‘stem 2’ region of the traerRNA.

Various aspects of the disclosure provide methods and compositions for improved guide RNA (including pegRNA) stability via chemical modifications. Braasch, D. A., Jensen, S., Liu, Y., Kaur, K., Arar, K., White, M. A., et al. (2003). RNA interference in mammalian cells by chemically-modified RNA. Biochemistry 42, 7967-7975. doi: 10.1021/bi0343774. Chiu, Y. L., and Rana, T. M. (2003). siRNA function in RNAi: a chemical modification analysis. RNA 9, 1034-1048. doi: 10.1261/rna.5103703. Behlke, M. A. (2008). Chemical modification of siRNAs for in vivo use. Oligonucleotides 18, 305-319. doi: 10.1089/oli.2008.0164. Bennett, C. F., and Swayze, E. E. (2010). RNA targeting therapeutics: molecular mechanisms of antisense oligonucleotides as a therapeutic platform. Annu. Rev. Pharmacol. Toxicol. 50, 259-293. doi: 10.1146/annurev.pharmtox.010909.105654. Deleavey, G. F., and Damha, M. J. (2012). Designing chemically modified oligonucleotides for targeted gene silencing. Chem. Biol. 19, 937-954. doi: 10.1016/j.chembiol.2012.07.011. Lennox, K. A., and Behlke, M. A. (2020). Chemical modifications in RNA interference and CRISPR/Cas genome editing reagents. Methods Mol. Biol. 2115, 23-55. doi: 10.1007/978-1-0716-0290-4_2.

For instance, Hendel et al. improved guide RNA stability by chemically modifying gRNA ends to reduce degradation by exonucleases, RNA nuclease. Hendel, A., Bak, R. O., Clark, J. T., Kennedy, A. B., Ryan, D. E., Roy, S., et al. (2015a). Chemically modified guide RNAs enhance CRISPR-Cas genome editing in human primary cells. Nat. Biotechnol. 33, 985-989. doi: 10.1038/nbt.3290. Chemical modifications of gRNAs may enable more efficient and safer gene-editing in primary cells suitable for clinical applications.

A review of types of chemical modifications are provided in Allen, Daniel et al. “Using Synthetically Engineered Guide RNAs to Enhance CRISPR Genome Editing Systems in Mammalian Cells.” Frontiers in genome editing vol. 2 617910. 28 Jan. 2021, doi:10.3389/fgeed.2020.617910.

Accordingly, in various embodiments of the present disclosure, the genome editing system comprising a guide RNA (including pegRNA) and further comprises one or more chemical modifications selected from, but not limited to the modifications in the above table.

In exemplary embodiments, chemical modifications to the guide RNA (including pegRNA) include modifications on the ribose rings and phosphate backbone of guide RNA (including pegRNA) and modifications at the 2′OH include 2′-O-Me, 2′-F, and 2′F-ANA. More extensive ribose modifications include 2′F-4′-Cα-OMe and 2′,4′-di-Cα-OMe combine modification at both the 2′ and 4′ carbons. Phosphodiester modifications include sulfide-based Phosphorothioate (PS) or acetate-based phosphonoacetate alterations. Combinations of the ribose and phosphodiester modifications have given way to formulations such as 2′-O-methyl 3′phosphorothioate (MS), or 2′-O-methyl-3′-thioPACE (MSP), and 2′-O-methyl-3′-phosphonoacetate (MP) RNAs. Locked and unlocked nucleotides such as locked nucleic acid (LNA), bridged nucleic acids (BNA), S-constrained ethyl (cEt), and unlocked nucleic acid (UNA) are examples of sterically hindered nucleotide modifications. Modifications to make a phosphodiester bond between the 2′ and 5′ carbons (2′,5′-RNA) of adjacent RNAs as well as a butane 4-carbon chain link between adjacent RNAs have been described.

### E. Additional Components and Aspects

In addition to the above LNPs and cargoes, including (A) nucleic acid payloads, (B) linear mRNA payloads, circular mRNA payloads, and (D) gene editing systems, the present disclosure provides additional optional LNP cargo components and tools that may be included as appropriate in the LNP gene editing systems described herein. The following optional components and tools may be combined in any combination as appropriate depending upon the particular gene editing system being delivered by the herein disclosed LNP-based gene editing systems.

### IV. Encoded Products of Payload mRNA (e.g., Nucleobase Editing Systems and Therapeutic Proteins)

### A. Polypeptides, Peptides, and Proteins

The LNP-based nucleobase editing systems and therapeutics described herein comprise one or more RNA payloads (e.g., linear or circular mRNA) which may comprise one or more coding regions that encode one or more products of interest. The one or more coding regions may encode a polypeptide, peptide and/or protein. As used herein, the term “polypeptide” generally refers to polymers of amino acids linked by peptide bonds and embraces “protein” and “peptides.” Polypeptides for the present disclosure include all polypeptides, proteins and/or peptides known in the art. Non-limiting categories of polypeptides include antigens, antibodies, antibody fragments, cytokines, peptides, hormones, enzymes, oxidants, antioxidants, synthetic polypeptides, and chimeric polypeptides, receptor, enzymes, hormones, transcription factors, ligands, membrane transporters, structural proteins, nucleases, or a component, variant or fragment (e.g., a biologically active fragment) thereof.

As used herein, the term “peptide” generally refers to shorter polypeptides of about 50 amino acids or less. Peptides with only two amino acids may be referred to as “dipeptides.” Peptides with only three amino acids may be referred to as “tripeptides.” Polypeptides generally refer to polypeptides with from about 4 to about 50 amino acids. Peptides may be obtained via any method known to those skilled in the art. In some embodiments, peptides may be expressed in culture. In some embodiments, peptides may be obtained via chemical synthesis (e.g., solid phase peptide synthesis).

In some embodiments, the RNA payloads (e.g., linear and/or circular mRNA payloads encoding one or more encoded products of interest), e.g., the originator constructs and benchmark constructs described herein, may encode a simple protein which upon hydrolysis yields the amino acids and occasionally small carbohydrate compounds. Non-limiting examples of simple proteins include albumins, albuminoids, globulins, glutelins, histones and protamines.

In some embodiments, the RNA payloads (e.g., linear and/or circular mRNA payloads encoding one or more encoded products of interest), e.g., the originator constructs and benchmark constructs described herein, may encode a simple protein associated with a non-protein. Non-limiting examples of conjugated proteins include, glycoproteins, hemoglobins, lecithoproteins, nucleoproteins, and phosphoproteins.

In some embodiments, the RNA payloads (e.g., linear and/or circular mRNA payloads encoding one or more encoded products of interest), e.g., the originator constructs and benchmark constructs described herein, may encode a protein that is derived from a simple or conjugated protein by chemical or physical means. Non-limiting examples of derived proteins include denatured proteins and peptides.

In some embodiments, the polypeptide, protein or peptide may be unmodified.

In some embodiments, the polypeptide, protein or peptide may be modified. Types of modifications include, but are not limited to, phosphorylation, glycosylation, acetylation, ubiquitylation/sumoylation, methylation, palmitoylation, quinone, amidation, myristoylation, pyrrolidone carboxylic acid, hydroxylation, phosphopantetheine, prenylation, GPI anchoring, oxidation, ADP-ribosylation, sulfation, S-nitrosylation, citrullination, nitration, gamma-carboxyglutamic acid, formylation, hypusine, topaquinone (TPQ), bromination, lysine topaquinone (LTQ), tryptophan tryptophylquinone (TTQ), iodination, and cysteine tryptophylquinone (CTQ). In some aspects, the polypeptide, protein or peptide may be modified by a post-transcriptional modification which can affect its structure, subcellular localization, and/or function.

In some embodiments, the polypeptide, protein or peptide may be modified using phosphorylation. Phosphorylation, or the addition of a phosphate group to serine, threonine, or tyrosine residues, is one of most common forms of protein modification. Protein phosphorylation plays an important role in fine tuning the signal in the intracellular signaling cascades.

In some embodiments, the polypeptide, protein or peptide may be modified using ubiquitination which is the covalent attachment of ubiquitin to target proteins. Ubiquitination-mediated protein turnover has been shown to play a role in driving the cell cycle as well as in protein-degradation-independent intracellular signaling pathways.

In some embodiments, the polypeptide, protein or peptide may be modified using acetylation and methylation which can play a role in regulating gene expression. As a non-limiting example, the acetylation and methylation could mediate the formation of chromatin domains (e.g., euchromatin and heterochromatin) which could have an impact on mediating gene silencing.

In some embodiments, the polypeptide, protein or peptide may be modified using glycosylation. Glycosylation is the attachment of one of a large number of glycan groups and is a modification that occurs in about half of all proteins and plays a role in biological processes including, but not limited to, embryonic development, cell division, and regulation of protein structure. The two main types of protein glycosylation are N-glycosylation and O-glycosylation. For N-glycosylation the glycan is attached to an asparagine and for O-glycosylation the glycan is attached to a serine or threonine.

In some embodiments, the polypeptide, protein or peptide may be modified using sumoylation. Sumoylation is the addition of SUMOs (small ubiquitin-like modifiers) to proteins and is a post-translational modification similar to ubiquitination.

In other embodiments, the RNA payloads (e.g., linear and/or circular mRNA payloads encoding one or more encoded products of interest), e.g., the originator constructs and benchmark constructs described herein, may encode a therapeutic protein, such as those exemplified below.

In other embodiments, the RNA payloads (e.g., linear and/or circular mRNA payloads encoding one or more products of interest), e.g., the originator constructs and benchmark constructs described herein, may encode a gene editing system, such as those exemplified herein. As used herein, a “nucleobase editing system” is a protein, DNA, or RNA composition capable of making edits, modifications or alterations to one or more targeted genes of interest. According to the present invention, one or more nucleobase editing system currently being marketed or in development may be encoded by the RNA payloads (e.g., linear and/or circular mRNA payloads encoding one or more encoded products of interest) described herein of the present invention.

### B. Fusion Proteins

In some embodiments, the polypeptide products (e.g., nucleobase editing systems and/or therapeutic proteins) of the RNA payload disclosed herein may be in the form of a fusion protein. Thus, the encoded polypeptides may include two or more proteins (e.g., protein and/or protein fragment) joined together, e.g., by a linker. In some embodiments, the fusion partner can provide an additional function to the encode polypeptide product, such as, but not limited to intracellular targeting, signaling, enzymatic function, stability, scaffolds, enhanced immunogenicity (in the case where the polypeptide encoded by the RNA payload is a nucleobase editing system). The disclosure contemplates that the polypeptide products (e.g., nucleobase editing systems and/or therapeutic proteins) of the RNA payload disclosed herein may be fused to any useful fusion partner known in the art.

### C. Linkers and Cleavable Peptides

In some embodiments, the mRNA payloads of the disclosure encode more than one polypeptide, referred to herein as fusion proteins. In some embodiments, the mRNA further encodes a linker located between at least one or each domain of the fusion protein.

The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one nucleobase editing system component/polypeptide separately within the same molecule) may be suitable for use as provided herein.

### D. Functional Domains

In some embodiments, the polypeptides encoded by the RNA payloads described herein may further comprise additional sequences or functional domains. For example, the nucleobase editing system polypeptides of the present disclosure may comprise one or more linker sequences. In some embodiments, the nucleobase editing system polypeptide may comprise a polypeptide tag, such as an affinity tag (chitin binding protein (CBP), maltose binding protein (MBP), glutathione-S-transferase (GST), SBP-tag, Strep-tag, AviTag, Calmodulin-tag); solubilization tag; chromatography tag (polyanionic amino acid tag, such as FLAG-tag); epitope tag (short peptide sequences that bind to high-affinity antibodies, such as V5-tag, Myc-tag, VSV-tag, Xpress tag, E-tag, S-tag, and HA-tag); fluorescence tag (e.g., GFP). In some embodiments, the nucleobase editing system peptide may comprise an amino acid tag, such as one or more lysines, histidines, or glutamates, which can be added to the polypeptide sequences (e.g., at the N-terminal or C-terminal ends). Lysines can be used to increase peptide solubility or to allow for biotinylation. Protein and amino acid tags are peptide sequences genetically grafted onto a recombinant protein. Sequence tags are attached to proteins for various purposes, such as peptide purification, identification, or localization, for use in various applications including, for example, affinity purification, protein array, western blotting, immunofluorescence, and immunoprecipitation. Such tags are subsequently removable by chemical agents or by enzymatic means, such as by specific proteolysis or intein splicing.

Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

### E. Codon Optimization

The LNP-based nucleobase editing systems and therapeutics described herein may comprise one or more RNA payloads (e.g., linear or circular mRNA) having nucleotide sequences which may be codon optimized.

For example, a nucleotide sequence (e.g., as part of an RNA payload) encoding a nucleobase editing system of the disclosure is codon optimized. Codon optimization methods are known in the art. For example, a protein encoding sequence of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the protein encoding sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a nucleobase editing enzyme). In some embodiments, a codon optimized sequence shares less than 90% sequence identity to a naturally-occurring or wild-type sequence (e.g., a naturally-occurring or wild-type mRNA sequence encoding a nucleobase editing enzyme). In some embodiments, a codon optimized sequence shares less than 85% sequence identity to a naturally-occurring or wild-type sequence (e.g., a naturally-occurring or wild-type mRNA sequence encoding a nucleobase editing enzyme). In some embodiments, a codon optimized sequence shares less than 80% sequence identity to a naturally-occurring or wild-type sequence (e.g., a naturally-occurring or wild-type mRNA sequence encoding a nucleobase editing enzyme). In some embodiments, a codon optimized sequence shares less than 75% sequence identity to a naturally-occurring or wild-type sequence (e.g., a naturally-occurring or wild-type mRNA sequence encoding a nucleobase editing enzyme).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a nucleobase editing enzyme). In some embodiments, a codon optimized sequence shares between 65% and 75% or about 80% sequence identity to a naturally-occurring or wild-type sequence (e.g., a naturally-occurring or wild-type mRNA sequence encoding a nucleobase editing enzyme).

When transfected into mammalian cells, the modified mRNA payloads have a stability of between 12-18 hours, or greater than 18 hours, e.g., 24, 36, 48, 60, 72, or greater than 72 hours.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### V. LNP Pharmaceutical Compositions

The LNP-based nucleobase editing systems, RNA therapeutics and pharmaceutical compositions thereof described herein can be formulate using one or more excipients to: (1) increase stability; (2) increase cell transfection or transduction; (3) permit the sustained or delayed expression of the payload; (4) alter the biodistribution (e.g., target the nucleobase editing systems to specific tissues or cell types); (5) increase the translation of encoded protein; (6) alter the release profile of encoded protein; and/or (7) allow for regulatable expression of an RNA payload expression product.

Formulations can include, without limitation, saline, liposomes, lipid nanoparticles, polymers, peptides, proteins, cells transfected with viral vectors (e.g., for transfer or transplantation into a subject) and combinations thereof.

Formulations of the LNP-based nucleobase editing systems, RNA therapeutics and pharmaceutical compositions thereof described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient (e.g., encapsulated LNP with an mRNA payload expressing a protein of interest) with an excipient and/or one or more other accessory ingredients. As used herein, the phrase “active ingredient” can refer to an LNP encapsulated with a payload mRNA, as well as to the mRNA payload construct itself, including originator constructs and benchmark construct as described herein.

Formulations of the encapsulated LNPs, the payload mRNA constructs, and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, formulations described herein may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

In one embodiment, the formulations described herein comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide—Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, Dl-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate—Butyl Methacrylate—Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O F1-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa.S); Hypromellose 2910 (15000 Mpa.S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate—Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol—Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa.S); Methylcelluloses; Methylchloroisothiazolinone; Methylene Blue; Methylisothiazolinone; Methylparaben; Microcrystalline Wax; Mineral Oil; Mono and Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride):Sebacic Acid; Poly(Dimethylsiloxane/Methylvinylsiloxane/Methylhydrogensiloxane) Dimethylvinyl Or Dimethylhydroxy Or Trimethyl Endblocked; Poly(Dl-Lactic-Co-Glycolic Acid), (50:50; Poly(Dl-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquatemium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, Dl-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

In some embodiments, formulations disclosed herein may include cations or anions. The formulations include metal cations such as, but not limited to, Zn²⁺, Ca²⁺, Cu²⁺, Mn²⁺, Mg²⁺, and combinations thereof. As a non-limiting example, formulations may include polymers and complexes with a metal cation.

Formulations of the disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

In some embodiments, the payloads are encapsulated in nanoparticles (e.g., LNPs) for delivery. In embodiments, a nanoparticle can include an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In certain embodiments, the lipid component of the nanoparticle composition includes about 20 mol % to about 45 mol % ionizable lipid, about 30 mol % to about 60 mol % phospholipid, about 10 mol % to about 30 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid, about 5 mol % to about 25 mol % phospholipid, about 30 mol % to about 40 mol % structural lipid, and about 0 mol % to about 10 mol % of PEG lipid, provided that the total mol % does not exceed 100%. In some embodiments, the lipid component of the nanoparticle composition includes about 30 mol % to about 40 mol % ionizable lipid, about 35 mol % to about 45 mol % phospholipid, about 20 mol % to about 30 mol % structural lipid, and about 0.5 mol % to about 5 mol % of PEG lipid, provided that the total mol % does not exceed 100%. In a particular embodiment, the lipid component includes about 50 mol % ionizable lipid, about 10 mol % phospholipid, about 38.5 mol % structural lipid, and about 1.5 mol % of PEG lipid. In another particular embodiment, the lipid component includes about 40 mol % ionizable lipid, about 20 mol % phospholipid, about 38.5 mol % structural lipid, and about 1.5 mol % of PEG lipid. In another particular embodiment, the lipid component includes about 48.5 mol % ionizable lipid, about 10 mol % phospholipid, about 40 mol % structural lipid, and about 1.5 mol % of PEG lipid. In another particular embodiment, the lipid component includes about 48.5 mol % ionizable lipid, about 10 mol % phospholipid, about 39 mol % structural lipid, and about 2.5 mol % of PEG lipid. In another particular embodiment, the lipid component includes about 33 mol % ionizable lipid, about 40 mol % phospholipid, about 25 mol % structural lipid, and about 2 mol % of PEG lipid. In some embodiments, the phospholipid is DOPE or DSPC. In some embodiments, the phospholipid is DSPC. In some embodiments, the phospholipid is a sphingolipid. In some embodiments, the phospholipid is a sphingomyelin. In other embodiments, the PEG lipid is PEG-DMG (eg. PEG2K-DMG). In other embodiments, the PEG lipid is PEG-DSPE (eg. PEG2K-DSPE). In other embodiments, the PEG lipid is PEG-DMPE (eg. PEG2K-DMPE). In other embodiments, the structural lipid is cholesterol. In other embodiments, the PEG lipid is PEG-DMG and/or the structural lipid is cholesterol. In some embodiments, the PEG lipids is PEG2K-DMG, the structural lipid is cholesterol, and the phospholipid is DSPC. In some embodiments, the PEG lipids is PEG2K-DMG, the structural lipid is cholesterol, and the phospholipid is sphingomyelin. In some embodiments, the PEG lipids is PEG-DMG, the structural lipid is cholesterol, and the phospholipid is a mixture of DSPC and sphingomyelin. In certain embodiments, the LNP comprises about 33 mol % ionizable lipid (eg. at least one ionizable lipid of Formula AX described herein), about 40 mol % of a sphingolipid, about 25 mol % cholesterol and about 2 mol % PEG2K-DMG. In some embodiments, the PEG lipids is PEG2K-DSPE, the structural lipid is cholesterol, and the phospholipid is DSPC. In some embodiments, the PEG lipids is PEG2K-DSPE, the structural lipid is cholesterol, and the phospholipid is sphingomyelin. In certain embodiments, the LNP comprises about 33 mol % ionizable lipid, about 20 mol % of a sphingolipid, about 20 mol % of a non-sphingolipid phospholipid, about 25 mol % cholesterol and about 2 mol % of a PEGylated lipid. In certain embodiments, the LNP comprises about 43 mol % ionizable lipid, about 15 mol % of a sphingolipid, about 15 mol % of a non-sphingolipid phospholipid, about 25 mol % cholesterol and about 2 mol % of a PEGylated lipid. In certain embodiments, the LNP comprises about 33 mol % ionizable lipid, about 25 mol % of a sphingolipid, about 15 mol % of a non-sphingolipid phospholipid, about 25 mol % cholesterol and about 2 mol % of a PEGylated lipid. In certain embodiments, the LNP comprises about 33 mol % ionizable lipid, about 15 mol % of a sphingolipid, about 25 mol % of a non-sphingolipid phospholipid, about 25 mol % cholesterol and about 2 mol % of a PEGylated lipid. In some embodiments, the PEG lipid is PEG2K-DSPE, the structural lipid is cholesterol, and the phospholipid is a mixture of DSPC and sphingomyelin. In some embodiments, the PEG lipid is PEG2K-DMG, the structural lipid is cholesterol, and the phospholipid is a mixture of DSPC and sphingomyelin. In certain embodiments, the LNP comprises about 48.5 mol % ionizable lipid, about 10 mol % of a phospholipid (such as DSPC), about 40 mol % cholesterol and about 1.5 mol % PEG2K-DSPE. In certain embodiments, the LNP comprises about 48.5 mol % ionizable lipid, about 10 mol % of a phospholipid (such as DSPC), about 40 mol % cholesterol and about 1.5 mol % PEG2K-DMG. In certain embodiments, the LNP comprises about 48.5 mol % ionizable lipid, about 10 mol % of a phospholipid (such as DSPC), about 39 mol % cholesterol and about 2.5 mol % PEG2K-DSPE. The amount of active agent in a nanoparticle composition may depend on the size, composition, desired target and/or application, or other properties of the nanoparticle composition as well as on the properties of the active agent. For example, the amount of active agent useful in a nanoparticle composition may depend on the size, sequence, and other characteristics of the active agent. The relative amounts of active agent and other elements (e.g., lipids) in a nanoparticle composition may also vary. In some embodiments, the wt/wt ratio of the lipid component to a polynucleotide or an enzyme in a nanoparticle composition is from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a polynucleotide or an enzyme in a nanoparticle composition may, for example, be measured using absorption spectroscopy (e.g., ultraviolet-visible spectroscopy).

In some embodiments, a nanoparticle composition of the present disclosure is formulated to provide a specific N:P ratio. The N:P ratio of the composition refers to the molar ratio of nitrogen atoms in one or more lipids to the number of phosphate groups in an RNA active agent (e.g., a linear or circular mRNA payload). In general, a lower N:P ratio is preferred. The one or more enzymes, lipids, and amounts thereof is selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio is from about 2:1 to about 8:1. In other embodiments, the N:P ratio is from about 5:1 to about 8:1. For example, the N:P ratio is about 5.0:1, about 5.5:1, about 5.67:1, about 6.0:1, about 6.5:1, or about 7.0:1.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

In some embodiments, the mean size of a nanoparticle composition is between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition is from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition is from about 70 nm to about 100 nm. In a particular embodiment, the mean size is about 80 nm. In other embodiments, the mean size is about 100 nm.

In some embodiments, the LNPs of the present disclosure can be characterized by their shape. In some embodiments, the LNPs are essentially spherical. In some embodiments, the LNPs are essentially rod-shaped (i.e., cylindrical). In some embodiments, the LNPs are essentially disk shaped.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a nanoparticle composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition is from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of a payload describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency is at least 80%. In certain embodiments, the encapsulation efficiency is at least 90%.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

A nanoparticle composition may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro: Lippincott, Williams & Wilkins, Baltimore, Md., 2006).

### VI. Routes of Administration

The LNP-based nucleobase editing systems, RNA therapeutics and pharmaceutical compositions thereof described herein may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions may be administered in a way which allows them to cross the blood-brain barrier, vascular barrier, or other epithelial barrier. The originator constructs, benchmark constructs, and targeting systems may be administered in any suitable form, either as a liquid solution or suspension, as a solid form suitable for liquid solution or suspension in a liquid solution. The originator constructs, benchmark constructs, and targeting systems may be formulated with any appropriate and pharmaceutically acceptable excipient.

In some embodiments, the originator constructs, benchmark constructs, and targeting systems may be delivered to a subject via a single route administration.

In some embodiments, the originator constructs, benchmark constructs, and targeting systems may be delivered to a subject via a multi-site route of administration. A subject may be administered at 2, 3, 4, 5, or more than 5 sites.

In some embodiments, a subject may be administered the originator constructs, benchmark constructs, and targeting systems using a bolus infusion.

In some embodiments, a subject may be administered originator constructs, benchmark constructs, and targeting systems using sustained delivery over a period of minutes, hours, or days. The infusion rate may be changed depending on the subject, distribution, formulation or another delivery parameter.

In some embodiments, the originator constructs, benchmark constructs, and targeting systems may be delivered by intramuscular delivery route. Non-limiting examples of intramuscular administration include an intravenous injection or a subcutaneous injection.

In some embodiments, the originator constructs, benchmark constructs, and targeting systems may be delivered by oral administration. Non-limiting examples of oral delivery include a digestive tract administration and a buccal administration.

In some embodiments, the originator constructs, benchmark constructs, and targeting systems may be delivered by intraocular delivery route. A non-limiting example of intraocular delivery include an intravitreal injection.

In some embodiments, the originator constructs, benchmark constructs, and targeting systems may be delivered by intranasal delivery route. Non-limiting examples of intranasal delivery include nasal drops or nasal sprays.

In some embodiments, the originator constructs, benchmark constructs, and targeting systems may be administered to a subject by peripheral injections. Non-limiting examples of peripheral injections include intraperitoneal, intramuscular, intravenous, conjunctival, or joint injection.

In some embodiments, the originator constructs, benchmark constructs, and targeting systems may be delivered by injection into the cerebrospinal fluid. Non-limiting examples of delivery to the cerebrospinal fluid include intrathecal and intracerebroventricular administration.

In some embodiments, the originator constructs, benchmark constructs, and targeting systems may be delivered by systemic delivery. As a non-limiting example, the systemic delivery may be by intravascular administration.

In some embodiments, the originator constructs, benchmark constructs, and targeting systems may be administered to a subject by intracranial delivery.

In some embodiments, the originator constructs, benchmark constructs, and targeting systems may be administered to a subject by intraparenchymal administration.

In some embodiments, the originator constructs, benchmark constructs, and targeting systems may be administered to a subject by intramuscular administration.

In some embodiments, the originator constructs, benchmark constructs, and targeting systems are administered to a subject and transduce muscle of a subject. As a non-limiting example, the originator constructs, benchmark constructs, and targeting systems are administered by intramuscular administration.

In some embodiments, the originator constructs, benchmark constructs, and targeting systems may be administered to a subject by intravenous administration.

In some embodiments, the originator constructs, benchmark constructs, and targeting systems may be administered to a subject by subcutaneous administration.

In some embodiments, the originator constructs, benchmark constructs, and targeting systems may be administered to a subject by topical administration.

In some embodiments, the originator constructs, benchmark constructs, and targeting systems may be delivered by more than one route of administration.

The originator constructs, benchmark constructs, and targeting systems described herein may be co-administered in conjunction with one or more originator constructs, benchmark constructs, targeting systems, or therapeutic agents or moieties.

In some embodiments, pharmaceutical compositions and/or formulations described herein may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

In some embodiments, pharmaceutical compositions and/or formulations described herein may be formulated for administration topically. The skin may be an ideal target site for delivery as it is readily accessible. Three routes are commonly considered to deliver pharmaceutical compositions and/or formulations described herein to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions).

In some embodiments, pharmaceutical compositions and/or formulations described herein may be delivered using a variety of dressings (e.g., wound dressings) or bandages (e.g., adhesive bandages) for conveniently and/or effectively carrying out methods described herein. Typically dressing or bandages may comprise sufficient amounts of pharmaceutical compositions and/or formulations described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, contemplated herein is the use of transdermal patches, which often have the added advantage of providing controlled delivery of pharmaceutical compositions and/or formulations described herein to the body. Such dosage forms may be prepared, for example, by dissolving and/or dispensing pharmaceutical compositions and/or formulations described herein in the proper medium. Alternatively, or additionally, rates may be controlled by either providing rate controlling membranes and/or by dispersing pharmaceutical compositions and/or formulations described herein in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

In some embodiments, pharmaceutical compositions and/or formulations described herein may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

In some embodiments, pharmaceutical compositions and/or formulations described herein may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

In some embodiments, pharmaceutical compositions and/or formulations described herein are formulated in depots for extended release.

In some embodiments, pharmaceutical compositions and/or formulations described herein are spatially retained within or proximal to target tissues. Provided are methods of providing pharmaceutical compositions and/or formulations described herein to target tissues of mammalian subjects by contacting target tissues (which comprise one or more target cells) with pharmaceutical compositions and/or formulations described herein under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions and/or formulations described herein that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of pharmaceutical compositions and/or formulations described herein administered to subjects are present intracellularly at a period of time following administration. For example, intramuscular injection to mammalian subjects may be performed using aqueous compositions comprising an active ingredient and one or more transfection reagents, and retention is determined by measuring the amount of active ingredient present in muscle cells.

In some embodiments, provided are methods for delivering pharmaceutical compositions and/or formulations described herein to target tissues of mammalian subjects, by contacting target tissues (comprising one or more target cells) with pharmaceutical compositions and/or formulations described herein under conditions such that they are substantially retained in such target tissues. Pharmaceutical compositions and/or formulations described herein comprise enough active ingredient such that the effect of interest is produced in at least one target cell. In some embodiments, pharmaceutical compositions and/or formulations described herein generally comprise one or more cell penetration agents, although “naked” formulations (such as without cell penetration agents or other agents) are also contemplated, with or without pharmaceutically acceptable carriers.

In some embodiments, pharmaceutical compositions and/or formulations described herein may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

In some embodiments, pharmaceutical compositions and/or formulations described herein may be administered nasally and/or intranasal. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

In some embodiments, pharmaceutical compositions and/or formulations described herein may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### VII. Methods of Use

The LNP-based compositions described herein may be used to deliver a nucleobase editing system to a cell or tissue of interest. In certain embodiments, the LNP-based compositions described herein are useful for executing one or more edits, modifications or alterations to one or more targeted genes of interest. In certain embodiments, the one or more edits, modifications or alterations to the one or more targeted genes of interest are capable of treating a disease or disorder in a patient in need thereof.

### A. Methods of Producing Polypeptides in Cells

The present disclosure provides methods of producing a polypeptide of interest in a mammalian cell. Methods of producing polypeptides involve contacting a cell with an LNP comprising a nucleobase editing system comprising an mRNA encoding a component of said nucleobase editing system and/or a formulation or composition thereof as described herein. Upon contacting the cell with the lipid nanoparticle, the mRNA may be taken up and translated in the cell to produce a polypeptide of interest, e.g., an enzyme component of the nucleobase editing system.

In general, the step of contacting a mammalian cell with a LNP including an mRNA encoding a polypeptide of interest may be performed in vivo, ex vivo, in culture, or in vitro. The amount of lipid nanoparticle contacted with a cell, and/or the amount of mRNA therein, may depend on the type of cell or tissue being contacted, the means of administration, the physiochemical characteristics of the lipid nanoparticle and the mRNA (e.g., size, charge, and chemical composition) therein, and other factors. In general, an effective amount of the lipid nanoparticle will allow for efficient polypeptide production in the cell. Metrics for efficiency may include polypeptide translation (indicated by polypeptide expression), level of mRNA degradation, and immune response indicators.

The step of contacting an LNP including an mRNA with a cell may involve or cause transfection. A phospholipid including in the lipid component of a LNP may facilitate transfection and/or increase transfection efficiency, for example, by interacting and/or fusing with a cellular or intracellular membrane. Transfection may allow for the translation of the mRNA within the cell.

In some embodiments, the lipid nanoparticles described herein is used therapeutically. For example, an mRNA included in an LNP may encode a polypeptide (e.g., in a translatable region) that enables editing, modifying, or altering a target polynucleotide sequence and therefore produces a therapeutic effect upon entry (e.g., transfection) into a cell.

### B. Therapeutic Methods Using LNPs Described Herein

Provided herein are therapeutic methods for treating a disease or disorder by using the LNP-based RNA compositions described herein to deliver one or more therapeutic agents encoded on a payload RNA (e.g., linear or circular mRNA payload) to a cell, organ, or tissue. Delivery may be in vitro or ex vivo to cells, or to a cell, tissue, or organ in vivo.

Delivery of a therapeutic and/or prophylactic to a cell involves administering a composition of the disclosure that comprises a LNP encapsulated with a payload RNA (e.g., a linear or circular RNA) that encodes a therapeutic and/or prophylactic, where administration of the composition involves contacting the cell with the composition. In some embodiments, a protein, cytotoxic agent, radioactive ion, chemotherapeutic agent, or nucleic acid (such as an RNA, e.g., mRNA) is delivered to a cell or organ. In some embodiments, the mRNA payload itself may be regarded as the therapeutic and/or prophylactic as it encodes a therapeutic and/or prophylactic. Upon contacting a cell with the lipid nanoparticle, a translatable mRNA may be translated in the cell to produce a polypeptide of interest. However, mRNAs that are substantially not translatable may also be delivered to cells. Substantially non-translatable mRNAs may be useful as nucleobase editing systems and/or may sequester translational components of a cell to reduce expression of other species in the cell.

In some embodiments, an LNP may target a particular type or class of cells (e.g., cells of a particular organ or system thereof). In some embodiments, a LNP including an RNA payload coding for a therapeutic and/or prophylactic of interest is specifically delivered to a mammalian liver, kidney, spleen, femur, or lung. “Specific delivery” to a particular class of cells, an organ, or a system or group thereof implies that a higher proportion of lipid nanoparticles including a therapeutic and/or prophylactic are delivered to the destination (e.g., tissue) of interest relative to other destinations, e.g., upon administration of an LNP to a mammal. In some embodiments, specific delivery may result in a greater than 2-fold, 5-fold, 10-fold, 15-fold, or 20-fold increase in the amount of therapeutic and/or prophylactic per 1 g of tissue of the targeted destination (e.g., tissue of interest, such as a liver) as compared to another destination (e.g., the spleen). In some embodiments, the tissue of interest is selected from the group consisting of a liver, kidney, a lung, a spleen, a femur, vascular endothelium in vessels (e.g., intra-coronary or intra-femoral) or kidney, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in an LNP. An mRNA may additionally or instead be used to direct the synthesis and extracellular localization of lipids, carbohydrates, or other biological moieties. Alternatively, other therapeutics and/or prophylactics or elements (e.g., lipids or ligands) of an LNP may be selected based on their affinity for particular receptors (e.g., low density lipoprotein receptors) such that a LNP may more readily interact with a target cell population including the receptors. In some embodiments, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand is a surface-bound antibody, which can permit tuning of cell targeting specificity. This is especially useful since highly specific antibodies can be raised against an epitope of interest for the desired targeting site. In some embodiments, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell. In some embodiments an estrogen receptor ligand, such as tamoxifen, can target cells to estrogen-dependent breast cancer cells that have an increased number of estrogen receptors on the cell surface. Other non-limiting examples of ligand/receptor interactions include CCR1 (e.g., for treatment of inflamed joint tissues or brain in rheumatoid arthritis, and/or multiple sclerosis), CCR7, CCR8 (e.g., targeting to lymph node tissue), CCR6, CCR9, CCR10 (e.g., to target to intestinal tissue), CCR4, CCR10 (e.g., for targeting to skin), CXCR4 (e.g., for general enhanced transmigration), HCELL (e.g., for treatment of inflammation and inflammatory disorders, bone marrow), Alpha4beta7 (e.g., for intestinal mucosa targeting), and VLA-4NCAM-1 (e.g., targeting to endothelium). In general, any receptor involved in targeting (e.g., cancer metastasis) can be harnessed for use in the methods and compositions described herein.

Targeted cells may include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

In some embodiments, an LNP may target hepatocytes. Apolipoproteins such as apolipoprotein E (apoE) have been shown to associate with neutral or near neutral lipid-containing lipid nanoparticles in the body, and are known to associate with receptors such as low-density lipoprotein receptors (LDLRs) found on the surface of hepatocytes. Thus, an LNP including a lipid component with a neutral or near neutral charge that is administered to a subject may acquire apoE in a subject's body and may subsequently deliver a therapeutic and/or prophylactic (e.g., an RNA) to hepatocytes including LDLRs in a targeted manner.

Lipid nanoparticles described herein are useful for treating a disease, disorder, or condition. In particular, such compositions are useful in treating a disease, disorder, or condition characterized by missing or aberrant protein or polypeptide activity. In some embodiments, a formulation of the disclosure that comprises an LNP including an mRNA encoding a missing or aberrant polypeptide is administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment of acute diseases, disorders, or conditions such as sepsis, stroke, and myocardial infarction. A therapeutic and/or prophylactic included in an LNP may also be capable of altering the rate of transcription of a given species, thereby affecting gene expression.

Diseases, disorders, and/or conditions characterized by dysfunctional or aberrant protein or polypeptide activity for which a composition may be administered include, but are not limited to, rare diseases, infectious diseases, cancer and proliferative diseases, genetic diseases (e.g., cystic fibrosis), muscle-related conditions, autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering a LNP including an RNA and a lipid component including a PEGylated lipid compound disclosed herein, a phospholipid (optionally unsaturated), optionally a second PEGylated lipid, and a structural lipid, wherein the RNA may be an mRNA encoding a polypeptide that antagonizes or otherwise overcomes an aberrant protein activity present in the cell of the subject.

### C. Cancer

In various embodiments, the LNP-based RNA therapeutics and pharmaceutical compositions thereof described herein may be used to treat cancer.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, neoplasm of the central nervous system (CNS), primary CNS lymphoma, tumor angiogenesis, spinal axis tumor, brain stem glioma, pituitary adenoma, epidermoid cancer, squamous cell cancer, T cell lymphoma, environmentally induced cancers including those induced by asbestos, other B cell malignancies, and combinations of said cancers. In some embodiments, the methods disclosed herein may be used to reduce the tumor size of a tumor derived from, for example, sarcomas and carcinomas, fibrosarcoma, myxosarcoma, liposarcoma, chondrosarcoma, osteogenic sarcoma, Kaposi's sarcoma, sarcoma of soft tissue, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinoma, lung cancer, colorectal cancer, squamous cell carcinoma, basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, carcinoma of the fallopian tubes, carcinoma of the endometrium, carcinoma of the cervix, carcinoma of the vagina, carcinoma of the vulva, carcinoma of the renal pelvis, CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). The particular cancer may be responsive to chemo- or radiation therapy or the cancer may be refractory. A refractor cancer refers to a cancer that is not amendable to surgical intervention and the cancer is either initially unresponsive to chemo- or radiation therapy or the cancer becomes unresponsive over time.

D. Co-Therapy with Another Therapeutic Agent

In other embodiments, LNP-based RNA therapeutics and pharmaceutical compositions thereof described herein may be co-administered in conjunction with another agent for treating a disease (e.g., muscle atrophy and/or muscle-wasting disease, disease of the CNS, or cancer). Such additional agents are as follows: agents that treat conditions related to acute or chronic muscle atrophy and/or a muscle-wasting disease, such as sarcopenia, cachexia (e.g., megestrol, Megace®, Megace ES®, somatropin, Serostim®, and Norditropin FlexPro Pen®), cancer (e.g., carboplatin, Adriamycin®, Adrucil®, etoposide, fluorouracil, doxorubicin, Paraplatin®, Cosmegen®, cyclophosphamide, Ethyol®, Leukeran®, Vincasar PFS®, vincristine, Etopophos®, Oncovin®, Toposar®, Hycamtin®, Ifex®, ifosfamide, Mustargen®, Velban®, VePesid®, vinblastine, amifostine, chlorambucil, dactinomycin, mechlorethamine, Tepadina®, thiotepa, topotecan, and fludarabine), congestive heart failure (e.g., Furosemide®, Lasix®, carvedilol, Coreg®, spironolactone, Lisinopril®, digoxin, Metoprolol Succinate ER®, Aldactone®, Accupril®, metoprolol, isosorbide mononitrate, Coumadin®, Cardizem®, warfarin, Altace®, amlodipine, Lanoxin®, Norvasc®, Toprol-XL®, Nitrostat®, Diovan®, Prinivil®, Zestril®, diltiazem, hydralazine, nitroglycerin, Ramipril®, Apresoline®, bisoprolol, enalapril, torsemide, Vasotec®, isosorbide dinitrate, Lotensin®, allopurinol, Atacand®, Coreg®, valsartan, benazepril, Cardizem CD®, Cartia XT®, Digox®, Entresto®, Qbrelis®, quinapril, Cardizem LA®, CaroSpir®, Demadex®, Digitek®, Dilacor®, Dilt-XR®, Diltia XT®, Diltzac®, eplerenone, Jantoven®, Matzim LA®, nifedipine, Nitrolingual Pumpspray®, NitroQuick®, Taztia XT®, Tiadylt ER®, Tiazac®, candesartan, captopril, Clinacort®, dobutamine, hydrochlorothiazide/lisinopril, Isordil, Kenalog-40®, Lanoxicaps®, milrinone, Minitran®, Monoket®, Nitrek®, Nitro-Bid®, Nitro-Dur®, Nitro-Time®, Nitrocot®, Nitrol Appli-Kit®, NitroMist®, Nitro TD Patch-A®, Prexxartan®, Transderm-Nitro®, triamcinolone, amiloride, BiDil®, Capoten®, Corlanor®, Dobutrex®, hydrochlorothiazide/spironolactone, Inspra®, ivabradine, Midamor®, Minipress®, perindopril, prazosin, Prinzide®, sacubitril/valsartan, trandolapril, Zestoretic®, Aceon®, Aldactazide®, amiloride/hydrochlorothiazide, Capozide®, Capozide®, Capozide 25/25®, Capozide 50/15®, Capozide 50/25®, captopril/hydrochlorothiazide, Cardene®, Cardene IV®, Cardene SR®, Dilatrate-SR®, enalapril/hydrochlorothiazide, fosinopril, hydralazine/isosorbide dinitrate, Isochron®, IsoDitrate®, Isordil Titradose®, Mavik®, Moduretic 5-50®, moexipril, Monopril®, Natrecor®, nesiritide, nicardipine, Nipride RTU®, Nitropress®, nitroprusside, Primacor®, Univasc®, and Vaseretic®), renal failure (e.g., furosemide, Lasix®, Demadex®, Edecrin®, torsemide, Sodium Edecrin®, and ethacrynic acid), chronic obstructive pulmonary disease (e.g., Symbicort®, prednisone, montelukast, Breo Ellipta®, Daliresp®, Anoro Ellipta®, budesonide/formoterol, Tudorza Pressair®, Rayos®, aclidinium, fluticasone/vilanterol, Incruse Ellipta®, umeclidinium/vilanterol, roflumilast, Stiolto Respimat®, guaifenesin/theophylline, levalbuterol, olodaterol/tiotropium, dyphylline, olodaterol, Striverdi Respimat®, umeclidinium, Xopenex HFA®, Xopenex®, fluticasone/umeclidinium/vilanterol, Trelegy Ellipta®, and Xopenex Concentrate®), severe burns (e.g., silver sulfadiazine, Silvadene®, lidocaine, Xylocaine Jelly®, Bactine®, Dermoplast®, AneCream®, Solarcaine Burn Relief®, Albuminar-25®, Aloe Vera Burn Relief Spray with Lidocaine®, Lidocream®, Nupercainal®, SSD®, Xylocaine Topical®, Garamycin®, Thermazene®, Albutein®, AneCream with Tegaderm®, benzocaine, CidalEaze®, DermacinRx Lido V Pak®, Eha Lotion®, LidaMantle®, Lidopac®, Lidopin®, LidoRx®, LidoRxKit®, Lidotrans®, Lidovex®, Lidozion®, Lidozol®, Medi-Quik Spray®, RadiaGuard®, Regenecare HA Spray®, Senatec®, Sulfamylon®, Topicaine®, Vancocin®, Bionect®, gentamicin, vancomycin, mafenide, albumin human, dibucaine, Flexbumin®, Human Albumin Grifols®, Nebcin®, sodium hyaluronate, Tobi®, Tobramycin®, Vancocin HCl®, Vancocin HCl Pulvules®, Albuminar-5®, Albuminar-20®, SSD AF®, Albuked®, Albuked 5®, Albuked 25®, Albumin-ZLB®, Alburx®, Buminate®, Hylira®, IPM Wound®, Kedbumin®, Plasbumin®, Plasbumin-5®, Plasbumin-25®, RadiaPlex®, Solarcaine First Aid Medicated Spray®, and Xclair®), an inflammatory muscle disease, myasthenia gravis (e.g., Mestinon®, pyridostigmine, Mestinon Timespan®, azathioprine, mycophenolate mofetil, Prostigmin®, neostigmine, Prostigmin Bromide®, Regonol®, immune globulin intravenous, Soliris®, ephedrine, and eculizumab), neuropathy, polio (e.g., amantadine), multiple sclerosis (e.g., Copaxone®, Gilenya®, Ampyra®, Tysabri®, Tecfidera®, Aubagio®, Rebif®, Avonex®, Betaseron®, Decadron®, Prednisone®, Avonex Pen®, glatiramer, interferon beta-1a, fingolimod, dalfampridine, Novantrone®, dimethyl fumarate, teriflunomide, Acthar®, dexamethasone, Extavia®, natalizumab, Imuran®, Dexamethasone Intensol®, interferon beta-1b, prednisolone, Plegridy®, Prelone®, Rebif Rebidose®, valacyclovir, azathioprine, Lemtrada®, ocrelizumab, alemtuzumab, corticotropin, cyclophosphamide, Glatopa®, H.P. Acthar Gel®, mitoxantrone, peginterferon beta-la, Azasan®, cladribine, daclizumab, De-Sone LA®, Dexpak Taperpak®, Millipred®, Millipred DP®, mycophenolate mofetil, Ocrevus®, Orapred®, PediaPred®, Veripred 20®, and Zinbryta®), anorexia nervosa (e.g., olanzapine and cyproheptadine), human immunodeficiency virus/acquired immune deficiency syndrome (e.g., non-nucleoside reverse transcriptase inhibitors, nucleoside analog reverse transcriptase inhibitors, and protease inhibitors), osteomalacia (e.g., vitamin D2, Drisdol®, ergocalciferol, Calciferol®, Calcidol®, Posture®, Ridactate®, calcium lactate, and calcium phosphate, tribasic), herniated disk, hypercalcemia, kwashiorkor, Creutzfeldt-Jakob disease or bovine spongiform encephalopathy (e.g., methylene blue, cefotaxime, and Claforan®), diabetes (e.g., Tresiba®, insulin degludec, insulin aspart/insulin degludec, and Ryzodeg 70/30®), amyotrophic lateral sclerosis (e.g., Rilutek®, riluzole, edaravone, and Radicava®), necrotizing vasculitis, abetalipoproteinemia, malabsorption syndrome, Legg-Calvé-Perthes disease, polymyositis (e.g., prednisone), Guillain-Barre syndrome, osteoarthritis (e.g., paracetamol, nonsteroidal anti-inflammatory drugs, antacid, COX-2 selective inhibitors, and glucocorticoids), and/or muscular dystrophy (e.g., deflazacort, eteplirsen, Emflaza®, Exondys 51®, mexiletine, phenytoin, procainamide, and nusinersen), such as Duchenne, Becker congenital, distal, myotonic, oculopharyngeal, Limb-Girdle, facioscapulohumeral, and/or Emery-Dreifuss muscular dystrophy.

In other embodiments, the LNP-based RNA therapeutics and pharmaceutical compositions thereof described herein may be co-administered with a co-stimulatory molecule in order to achieve an immune response. An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-IBB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), 0X40 ligand, PD-L², or programmed death (PD) LL A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CDI00 (SEMA4D), CD103, CD134, CD137, CD154, CD16, CD160 (BY55), CD 18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, IT GAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD1 la/CD18), MHC class I molecule, NKG2C, NKG2D, NKp30, NKp44, NKp46, NKp80 (KLRFl), 0X40, PAG/Cbp, PD-1, PSGL1, SELPLG (CD162), signaling lymphocytic activation molecule, SLAM (SLAMFI; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

As used herein, the following terms and phrases are intended to have the following meanings:

### A/an

The articles “a” and “an” are used herein to refer to one or to more than one (i.e., to at least one) of the grammatical object of the article. By way of example, “an element” means one element or more than one element.

### About

As used herein, the term “about” means acceptable variations within 20%, within 10% and within 5% of the stated value. In certain embodiments, “about” can mean a variation of +/−1%, 2%, 3%, 4%, 5%, 10% or 20%. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

### Adjuvant

As used herein, “adjuvant” means an agent that does not constitute a specific antigen, but modifies (Th1/Th2), boosts the strength and longevity of an immune response, and/or broadens the immune response to a concomitantly administered antigen.

### Administration

The term “administration” or “administering” as used herein includes all means of introducing the compounds or the pharmaceutical compositions to the subject in need thereof, including but not limited to, oral, intravenous, intramuscular, intraperitoneal, subcutaneous, transdermal, inhalation, buccal, ocular, sublingual, vaginal, rectal and the like.

Administration of the compound or the composition is suitably parenteral. For example, the compounds or the composition can be preferentially administered intravenously, but can also be administered intraperitoneally or via inhalation like is currently used in the clinic for liposomal amikacin in the treatment of Mycobacterium avium (see Shirley et al., Amikacin Liposome Inhalation Suspension: A Review in Mycobacterium avium Complex Lung Disease. Drugs. 2019 April; 79(5):555-562).

### Aliphatic

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule, or two points of attachment to the rest of the molecule, as would be apparent to a person of ordinary skill in the art based on the context of the relevant molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

### Alkenyl

As used herein, “alkenyl” means a straight chain, cyclic or branched aliphatic hydrocarbon having the specified number of carbon atoms and one or more double bonds including but not limited to diene, triene and tetraene unsaturated aliphatic hydrocarbons. The terms “alkenyl” and “alkynyl”, refer to unsaturated aliphatic groups analogous in length and possible substitution to the alkyls described above, but that contain at least one double or triple bond respectively. In one embodiment, the alkenyl contains one double bond. In another embodiment, the alkenyl contains two double bonds. In another embodiment, the alkenyl contains three double bonds.

### Alkenylenyl

The term “alkenylenyl” as used herein refers to a divalent radical of an alkenyl group. In one embodiment, the alkenylenyl is a divalent form of a C₂₋₁₂ alkenyl, i.e., a C₂-C₁₂ alkenylenyl. In one embodiment, the alkenylenyl is a divalent form of a C₂₋₆ alkenyl, i.e., a C₂-C₁₀ alkenylenyl. In one embodiment, the alkenylenyl is a divalent form of a C₂₋₁₄ alkenyl, i.e., a C₂-C₈ alkenylenyl. In one embodiment, the alkenylenyl is a divalent form of an unsubstituted C₂₋₆ alkenyl, i.e., a C₂-C₆ alkenylenyl. In another embodiment, the alkylenyl is a divalent form of an unsubstituted C₂₋₄ alkenyl, i.e., a C₂-C₄ alkenylenyl. Nonlimiting exemplary alkenylenyl groups include CH═CH—, CH₂CH═CH—, CH₂CH₂CH═CHCH₂—, and CH₂CH═CHCH₂CH═CHCH₂CH₂—.

### Alkoxyl

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O— alkenyl, and —O-alkynyl. Aroxy can be represented by —O-aryl or O-heteroaryl, wherein aryl and heteroaryl are as defined below. The alkoxy and aroxy groups can be substituted as described above for alkyl.

### Alkyl

As used herein, “alkyl” means a straight chain, cyclic or branched saturated aliphatic hydrocarbon having the specified number of carbon atoms.

### Alkylenyl

The term “alkylenyl” as used herein refers to a divalent radical of a straight-chain or branched-chain alkyl group. In one embodiment, the alkylenyl is a divalent form of a C₁₋₁₂ alkyl, i.e., a C₁-C₁₂ alkylenyl. In one embodiment, the alkylenyl is a divalent form of a C₂₋₆ alkyl, i.e., a C₁-C₁₀ alkylenyl. In one embodiment, the alkylenyl is a divalent form of a C₂₋₁₄ alkyl, i.e., a C₁-C₈ alkylenyl. In one embodiment, the alkylenyl is a divalent form of an unsubstituted C₁₋₆ alkyl, i.e., a C₁-C₆ alkylenyl. In another embodiment, the alkylenyl is a divalent form of an unsubstituted C₁₋₄ alkyl, i.e., a C₁-C₄ alkylenyl. Nonlimiting exemplary alkylenyl groups include CH₂—, CH₂CH₂—, CH₂CH₂CH₂—, CH₂CH(CH₃)CH₂—, —CH₂(CH₂)₂CH₂—, CH(CH₂)₃CH₂—, and CH₂(CH₂)₄CH₂—.

### Alkylthio

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups.

Alkylthio groups can be substituted as defined above for alkyl groups.

### Aralkyl

The term “aralkyl,” as used herein, refers to an alkyl group substituted with an aryl group (e.g., an aromatic or heteroaromatic group).

### Aryl

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

### Analogs

As used herein, “analogs” is meant to include polypeptide variants which differ by one or more amino acid alterations, for example, substitutions, additions or deletions of amino acid residues that still maintain one or more of the properties of the parent or starting polypeptide.

### Antibodies

As used herein, the term “antibody” is referred to in the broadest sense and specifically covers various embodiments including, but not limited to monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include VH and VL domains, scFvs, Fab, Fab′, F(ab′)2, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

### Antigen

As defined herein, the term “antigen” or “antibody generator” (“Ag”) refers to a composition, for example, a substance or agent which causes an immune response in an organism, e.g., causes the immune response of the organism to produce antibodies against the substance or agent, in particular, which provokes an adaptive immune response in an organism. Antigens can be any immunogenic substance including, in particular, proteins, polypeptides, polysaccharides, nucleic acids, lipids and the like. Exemplary antigens are derived from infectious agents. Such agents can include parts or subunits of infectious agents, for example, coats, coat components, e.g., coat protein or polypeptides, surface components, e.g., surface proteins or polypeptides, capsule components, cell wall components, flagella, fimbrae, and/or toxins or toxoids) of infectious agents, for example, bacteria, viruses, and other microorganisms. Certain antigens, for example, lipids and/or nucleic acids are antigenic, preferably, when combined with proteins and/or polysaccharides.

### Approximately

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

### Associated

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

### Bicyclic Ring

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

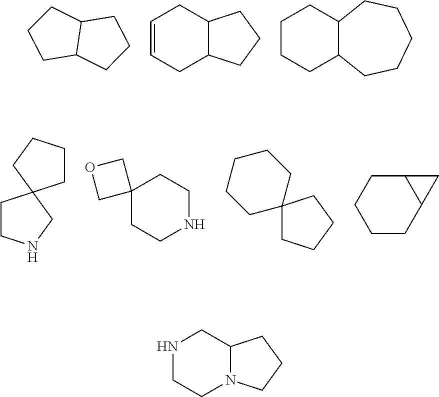

Exemplary bridged bicyclics include:

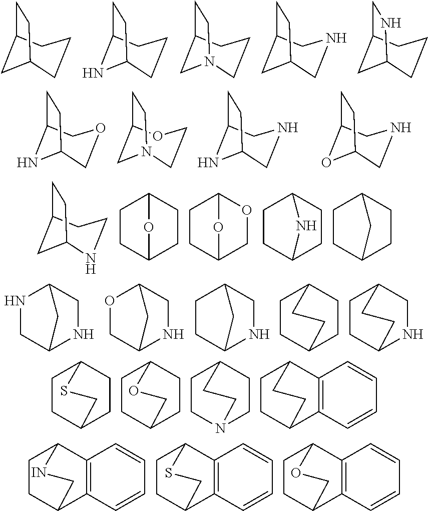

### Biologically Active

As used herein, the phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present invention may be considered biologically active if even a portion of the polynucleotides is biologically active or mimics an activity considered biologically relevant.

### Binding Domain

By “binding domain” it is meant a protein domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

### Bulge

As used herein, the term “bulge” refers to a small region of unpaired base(s) that interrupts a “stem” of base-paired nucleotides. The bulge may comprise one or two single-stranded or unbase-paired nucleotides joined at both ends by base-paired nucleotides of the stem. The bulge can be symmetrical (viz., the two unbase-paired single-stranded regions have the same number of nucleotides), or asymmetrical (viz., the unbase-paired single stranded region(s) have different or unequal numbers of nucleotides), or there is only one unbase-paired nucleotide on one strand. A bulge can be described as A/B (such as a “2/2 bulge,” or a “1/0 bulge”) wherein A represents the number of unpaired nucleotides on the upstream strand of the stem, and B represents the number of unpaired nucleotides on the downstream strand of the stem. An upstream strand of a bulge is more 5′ to a downstream strand of the bulge in the primary nucleotide sequence.

### CARs

As used herein, the term “chimeric antigen receptor” or “CAR” refers to an artificial chimeric protein comprising at least one antigen specific targeting region (ASTR), a transmembrane domain and an intracellular signaling domain, wherein the antigen specific targeting region comprises a full-length antibody or a fragment thereof. Any molecule that is capable of binding a target antigen with high affinity can be used in the ASTR of a CAR. The CAR may optionally have an extracellular spacer domain and/or a co-stimulatory domain. A CAR may also be used to generate a cytotoxic cell carrying the CAR.

### Carbocycle

The term “carbocycle,” as used herein, refers to an aromatic or non-aromatic ring in which each atom of the ring is carbon.

### Carbonyl

The term “carbonyl” is art-recognized and includes such moieties as can be represented by the general formula:

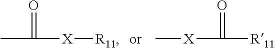

wherein X is a bond or represents an oxygen or a sulfur, and R¹¹ represents a hydrogen, an alkyl, a cycloalkyl, an alkenyl, a cycloalkenyl, or an alkynyl, R′₁₁ represents a hydrogen, an alkyl, a cycloalkyl, an alkenyl, a cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R¹ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

### Cargo or Payload

As used herein, the term “cargo” or “payload” can refer to one or more molecules or structures encompassed in a delivery vehicle for delivery to or into a cell or tissue. Non-limiting examples of cargo can include a nucleic acid (e.g., mRNA, such as a linear or a circular mRNA), a polypeptide, a peptide, a protein, a liposome, a label, a tag, a small chemical molecule, a large biological molecule, and any combinations thereof.

### Cationic Lipid

As used herein, “cationic lipid” refers to any of a number of lipid species which carry a net positive charge at a selective pH, such as physiological pH.

cDNA

As used hereing, the term “cDNA” refers to a strand of DNA copied from an RNA template, e.g., by a reverse transcriptase.

### Circular RNA

As used herein, the terms “circular RNA” or “circRNA” or “oRNA” equivalently refer to a RNA that forms a circular structure through covalent or non-covalent bonds.

### Co-Administration

As used herein the term “co-administration” or “co-administering” refers to administration of the LNP adjuvant and an agonist or antigen concurrently, i.e., simultaneously in time, or sequentially, i.e., administration of an LNP adjuvant, followed by administration of the agonist or antigen. That is, after administration of the LNP adjuvant, the agonist or antigen can be administered substantially immediately after the LNP adjuvant or the agonist or antigen can be administered after an effective time period after the LNP adjuvant; the effective time period is the amount of time given for realization of maximum benefit from the administration of the LNP adjuvant. An effective time period can be determined experimentally and can be generally within 1, 2, 3, 5, 10, 15, 20, 25, 30, 45 or 60 minutes.

### Complementary

As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

### Compound or Structure

The term “compound” or “structure,” as used herein, is meant to include all stereoisomers, geometric isomers, tautomers, and isotopes of the structures depicted. The compounds or structures described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds or structures of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H- isoindole, and IH- and 2H-pyrazole. Tautomeric forms can be in equilibrium or sterically locked into one form by appropriate substitution.

Compounds or structures of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g., have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. In some embodiments, alkyl groups are lower alkyls. In some embodiments, a substituent designated herein as alkyl is a lower alkyl.

### Comprising/Comprises

As used herein the term “comprising” or “comprises” is used in reference to compositions, methods, and respective component(s) thereof, that are present in a given embodiment, yet open to the inclusion of unspecified elements.

### Conservative Amino Acid Substitution

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

### Consisting Essentially of

As used herein the term “consisting essentially of” refers to those elements required for a given embodiment. The term permits the presence of additional elements that do not materially affect the basic and novel or functional characteristic(s) of that embodiment of the disclosure.

### Consisting of

The term “consisting of” refers to compositions, methods, and respective components thereof as described herein, which are exclusive of any element not recited in that description of the embodiment.

### Cycloalkylenyl

The term “cycloalkylenyl” as used herein refers to a divalent radical of a cycloalkyl group. In one embodiment, the cycloalkylenyl is a divalent form of a C₃₋₈ cycloalkyl, i.e., a C₃-C₈ cycloalkylenyl. Nonlimiting exemplary cycloalkylenyl groups include:

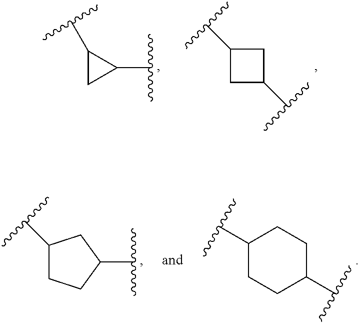

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

### Delivery

As used herein, “delivery” refers to the act or manner of delivering a compound, substance, entity, moiety, cargo or payload.

### Derivative

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

### DNA/RNA

As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

### DNA-Guided Nuclease or Nucleic Acid Programmable Nuclease

As used herein, an “DNA-guided nuclease” is a type of “programmable nuclease,” and a specific type of “nucleic acid-guided nuclease.” Equivalent terms for purposes of this disclosure include “nucleic acid programmable nuclease.” An example of a DNA-guided nuclease is reported in Varshney et al., DNA-guided genome editing using structure-guided endonucleases, Genome Biology, 2016, 17(1), 187, which may be used in the context of the present disclosure and is incorporated herein by reference. As used herein, the term “DNA-guided nuclease” or “DNA-guided endonuclease” refers to a nuclease that associates covalently or non-covalently with a guide RNA thereby forming a complex between the guide RNA and the DNA-guided nuclease. The guide RNA comprises a spacer sequence which comprises a nucleotide sequence having complementarity with a strand of a target DNA sequence. Thus, the DNA-guided nuclease is indirectly guided or programmed to localize to a specific site in a DNA molecule through its association with the guide RNA, which directly binds or anneals to a strand of the target DNA through its complementarity region via Watson-Crick base-pairing. A “nucleic acid programmable

### DNA-Guided DNA Binding Protein or Nucleic Acid Programmable DNA Binding Protein

As used herein, an “DNA-guided DNA binding protein” or “nucleic acid programmable DNA binding protein” has a similar meaning as a “DNA-guided nuclease” except that a “DNA binding protein” may include a nuclease but is not required to have a nuclease activity. By contrast to a nuclease, a nuclease domain (e.g., FokI) could be fused to a DNA binding protein top give rise to a DNA-guided nuclease as a fusion protein.

### DNA Regulatory Sequences

As used herein, the terms “DNA regulatory sequences,” “control elements,” and “regulatory elements,” can be used interchangeably herein to refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence and/or regulate translation of a mRNA into an encoded polypeptide.

### Domain

As used herein when referring to polypeptides the term “domain” refers to a motif of a polypeptide having one or more identifiable structural or functional characteristics or properties (e.g., binding capacity, serving as a site for protein-protein interactions).

### Donor Template DNA or Template DNA

By a “donor template DNA” or “donor DNA” or “template DNA” it is meant a single-stranded or double-stranded DNA to be incorporated at a site cleaved by a programmable nuclease (e.g., a CRISPR/Cas effector protein; a TALEN; a ZFN; a meganuclease) (e.g., after dsDNA cleavage, after nicking a target DNA, after dual nicking a target DNA, and the like). The donor DNA can contain sufficient homology to a genomic sequence at the target site, e.g. 70%, 80%, 85%, 90%, 95%, or 100% homology with the nucleotide sequences flanking the target site, e.g., within about 200 bases or less of the target site, e.g., within about 190 bases or less of the target site, e.g., within about 180 bases or less of the target site, e.g., within about 170 bases or less of the target site, e.g., within about 160 bases or less of the target site, e.g., within about 150 bases or less of the target site, e.g., within about 140 bases or less of the target site, e.g., within about 130 bases or less of the target site, e.g., within about 120 bases or less of the target site, e.g., within about 110 bases or less of the target site, e.g., within about 100 bases or less of the target site, e.g., within about 90 bases or less of the target site, e.g., within about 80 bases or less of the target site, e.g., within about 70 bases or less of the target site, e.g., within about 60 bases or less of the target site, e.g., 50 bases or less of the target site, e.g., within about 30 bases, within about 15 bases, within about 10 bases, within about 5 bases, or immediately flanking the target site, to support integration into the cut site. In certain embodiments, integration of the donor template DNA occurs by way of homology-directed repair between the donor and the genomic sequence to which it bears homology.

### Encapsulate

The terms “encapsulation” and “entrapped,” as used herein, refer to the incorporation or association of the mRNA, DNA, siRNA or other nucleic acid pharmaceutical agent in or with a lipidic nanoparticle. As used herein, the term “encapsulated” refers to complete encapsulation or partial encapsulation. A siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. A siRNA may comprise a sequence that is complementary to an mRNA sequence that encodes a gene or protein of interest.

### Encapsulation Efficiency

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of a polynucleotide are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Throughout the disclosure, chemical substituents described in Markush structures are represented by variables. Where a variable is given multiple definitions as applied to different Markush formulas in different sections of the disclosure, it is to be understood that each definition should only apply to the applicable formula in the appropriate section of the disclosure.

### Encode

As used herein the term “encode” refers broadly to any process whereby the information in a polymeric macromolecule is used to direct the production of a second molecule that is different from the first. The second molecule may have a chemical structure that is different from the chemical nature of the first molecule.

### Exosome

As used herein, the term “exosomes” refer to small membrane bound vesicles with an endocytic origin. Without wishing to be bound by theory, exosomes are generally released into an extracellular environment from host/progenitor cells post fusion of multivesicular bodies the cellular plasma membrane. As such, exosomes can include components of the progenitor membrane in addition to designed components (e.g. engineered TnpB editing system). Exosome membranes are generally lamellar, composed of a bilayer of lipids, with an aqueous inter-nanoparticle space.

### Features

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini or any combination thereof.

### Fusion

The term “fusion” as used herein as applied to a nucleic acid or polypeptide refers to two components that are defined by structures derived from different sources. For example, where “fusion” is used in the context of a fusion polypeptide (e.g., a fusion Cas9-RT protein), the fusion polypeptide includes amino acid sequences that are derived from different polypeptides. A fusion polypeptide may comprise either modified or naturally-occurring polypeptide sequences (e.g., a first amino acid sequence from a modified or unmodified Cas9-RT protein; and a second amino acid sequence from a modified or unmodified protein other than a Cas9-RT protein, etc.). Similarly, “fusion” in the context of a polynucleotide encoding a fusion polypeptide includes nucleotide sequences derived from different coding regions (e.g., a first nucleotide sequence encoding a modified or unmodified Cas9-RT protein; and a second nucleotide sequence encoding a polypeptide other than a Cas9-RT protein).

### Fusion Polypeptide

The term “fusion polypeptide” refers to a polypeptide which is made by the combination (i.e., “fusion”) of two otherwise separated segments of amino acid sequence, usually through human intervention.

### Formulation

As used herein, a “formulation” includes at least one compound, substance, entity, moiety, cargo or payload and a delivery agent.

### Fragment

A “fragment,” as used herein, refers to a portion. For example, fragments of proteins may comprise polypeptides obtained by digesting full-length protein isolated from cultured cells.

### Halogen

As used herein, “halogen” means Br, Cl, F and I.

### Heteroalkyl

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

### Heteroatom

The term “heteroatom” as used herein means an atom of any element other than carbon or hydrogen. Examples of heteroatoms are boron, nitrogen, oxygen, phosphorus, sulfur and selenium. Other useful heteroatoms include silicon and arsenic.

### Heterocyclyl

As used herein, “heterocyclyl” or “heterocycle” means a 4- to 10-membered aromatic or nonaromatic heterocycle containing from 1 to 4 heteroatoms selected from the group consisting of O, N and S, and includes bicyclic groups. “Heterocyclyl” therefore includes, the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof all of which are optionally substituted with one to three substituents selected from R″.

### Heterocycle

“Heterocycle” or “heterocyclic,” as used herein, refers to a cyclic radical attached via a ring carbon or nitrogen of a monocyclic or bicyclic ring containing 3-10 ring atoms, for example, from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

### Homology

As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids. Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

### Homology-Directed Repair

As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the targeted polynucleotide sequence.

### Identity

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

### Immunogenic

As used herein, the term “immunogenic” refers to a potential to induce an immune response to a substance. An immune response may be induced when an immune system of an organism or a certain type of immune cell is exposed to an immunogenic substance. The term “non-immunogenic” refers to a lack of or absence of an immune response above a detectable threshold to a substance. No immune response is detected when an immune system of an organism or a certain type of immune cell is exposed to a non-immunogenic substance. In some embodiments, a non-immunogenic circular polyribonucleotide as provided herein, does not induce an immune response above a pre-determined threshold when measured by an immunogenicity assay. In some embodiments, no innate immune response is detected when an immune system of an organism or a certain type of immune cell is exposed to a non-immunogenic circular polyribonucleotide as provided herein. In some embodiments, no adaptive immune response is detected when an immune system of an organism or a certain type of immune cell is exposed to a non-immunogenic circular polyribonucleotide as provided herein.

### Ionizable Lipid

As used herein “ionizable lipid” refers to any of a number of lipid species that carry a net positive charge at a selected pH.

### IRES

As used herein, the term “internal ribosome entry site” or “IRES” refers to an RNA sequence or structural element ranging in size form 10 nucleotides to 1,000 nucleotides or more which is capable of initiating translation of a polypeptide in the absence of a normal RNA cap structure. In other words, IRES are sequences that can recruit ribosomes and allow cap-independent translation, which can link two coding sequences in one bicistronic vector and allow the translation of both proteins. See Kozak M, “A second look at cellular mRNA sequences said to function as internal ribosome entry sites,” Nucleic Acids Research, 2005, Vol. 33: pp. 6593-6602 (incorporated herein by reference).

### Linker

As used herein, the term“linker” refers to a molecule linking or joining two other molecules or moieties. The linker can be an amino acid sequence in the case of a linker joining two fusion proteins. For example, a TnpB protein can be fused to an accessory protein (e.g., a deaminase, nuclease, ligase, reverse transcriptase, recombinase, etc.) by an amino acid linker sequence. The linker can also be a nucleotide sequence in the case of joining two nucleotide sequences together. For example, in the instant case, a reRNA at its 5′ and/or 3′ ends may be linked by a nucleotide sequence linker to one or more other functional nucleic acid molecules, such as guide RNAs or HDR donor molecules. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Lipid Conjugate

The term “lipid conjugate” refers to a conjugated lipid that inhibits aggregation of lipid particles. Such lipid conjugates include, but are not limited to, polysarcosine (see e.g. WO2021191265A1 which is herein incorporated by reference in its entirety for all purposes), polyamide oligomers (e.g., ATTA-lipid conjugates), PEG-lipid conjugates, such as PEG coupled to di alkyl oxy propyls, PEG coupled to diacylglycerols, PEG coupled to cholesterol, PEG coupled to phosphatidylethanolamines, PEG conjugated to ceramides (see, e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

### Lipid Nanoparticle (LNP)

The term “lipid nanoparticle”, or “LNP”, refers to particles having a diameter of from about 5 to 500 nm. In some embodiments, lipid nanoparticle refers to any lipid composition that can be used to deliver a prophylactic product, preferably vaccine antigens, including, but not limited to, liposomes or vesicles, wherein an aqueous volume is encapsulated by amphipathic lipid bilayers (e.g., single; unilamellar or multiple; multilamellar), or, in other embodiments, wherein the lipids coat an interior comprising a prophylactic product, or lipid aggregates or micelles, wherein the lipid encapsulated therapeutic product is contained within a relatively disordered lipid mixture. Except where noted, the lipid nanoparticle does not need to have antigen incorporated therein and may be used to deliver a prophylactic product when in the same formulation.

In some embodiments, the active agent (e.g., RNA payload encoding a polypeptide, such as an antigen or therapeutic protein) is encapsulated into the LNP. In some embodiments, the active agent can be an anionic compound, for example, but not limited to DNA, RNA, natural and synthetic oligonucleotides (including antisense oligonucleotides, interfering RNA and small interfering RNA), nucleoprotein, peptide, nucleic acid, ribozyme, DNA-containing nucleoprotein, such as an intact or partially deproteinated viral particles (virions), oligomeric and polymeric anionic compounds other than DNA (for example, acid polysaccharides and glycoproteins)). In some embodiments, the active agent can be intermixed with an adjuvant.

In a LNP vaccine product described herein, the active agent is generally contained in the interior of the LNP. In some embodiments, the active agent comprises a nucleic acid (e.g., a circular or linear mRNA). Typically, water soluble nucleic acids are condensed with cationic lipids or polycationic polymers in the interior of the particle and the surface of the particle is enriched in neutral lipids or PEG-lipid derivatives. Additional ionizable cationic lipid may also be at the surface and respond to acidification in the environment by becoming positively charged, facilitating endosomal escape. Lipid components of the herein disclosed LNPs are described herein.

Release of nucleic acids from LNP formulations, among other characteristics such as liposomal clearance and circulation half-life, can be modified by the presence of polyethylene glycol and/or sterols (e.g., cholesterol) or other potential additives in the LNP, as well as the overall chemical structure, including pKa of any ionizable cationic lipid included as part of the formulation.

### Liposome

As used herein “liposome” generally refers to a vesicle composed of lipids (e.g., amphiphilic lipids) arranged in one or more spherical bilayers or bilayers.

### Modified

As used herein “modified” refers to a changed state or structure of a molecule. Molecules may be modified in many ways including chemically, structurally, and functionally.

### Modulating

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

### Naturally-Occurring

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism that can be isolated from a source in nature is naturally occurring.

### Non-Homologous End Joining

As used herein, “non-homologous end joining (NHEJ)” refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

### Nuclear Localization Sequence (NLS)

As used herein, the term“nuclear localization sequence” or “NLS” refers to an amino acid sequence that promotes import of a protein (e.g., a RNA-guided nuclease) into the cell nucleus, for example, by nuclear transport. Nuclear localization sequences are known in the art. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences.

### Nuclease

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

### Nucleic Acid

As used herein, the term “nucleic acid” or “nucleic acid molecule” or “nucleic acid sequence” or “polynucleotide” generally refer to deoxyribonucleic or ribonucleic oligonucleotides in either single- or double-stranded form. The term may (or may not) encompass oligonucleotides containing known analogues of natural nucleotides. The term also may (or may not) encompass nucleic acid-like structures with synthetic backbones, see, e.g., Eckstein, 1991; Baserga et ah, 1992; Milligan, 1993; WO 97/03211; WO 96/39154; Mata, 1997; Strauss-Soukup, 1997; and Samstag, 1996. The term encompasses both ribonucleic acid (RNA) and DNA, including cDNA, genomic DNA, synthetic, synthesized (e.g., chemically synthesized) DNA, and/or DNA (or RNA) containing nucleic acid analogs. The nucleotides Adenine (A), Thymine (T), Guanine (G) and Cytosine (C) also may (or may not) encompass nucleotide modifications, e.g., methylated and/or hydroxylated nucleotides, e.g., Cytosine (C) encompasses 5-methylcytosine and 5-hydroxymethylcytosine.

### Nucleic Acid Loop

As used herein, the term “loop” in the polynucleotide refers to a single stranded stretch of one or more nucleotides, such as 2, 3, 4, 5, 6, 7, 8, 9, or 10 nucleotides, wherein the most 5′ nucleotide and the most 3′ nucleotide of the loop are each linked to a base-paired nucleotide in a stem.

### Nucleic Acid Stem

As used herein, the term “stem” refers to two or more base pairs, such as 3, 4, 5, 6, 7, 8, 9, 10, 11, 12, 13, 14, 15, 16, 17, 18, 19, 20 or more base pairs, formed by inverted repeat sequences connected at a “tip,” where the more 5′ or “upstream” strand of the stem bends to allows the more 3′ or “downstream” strand to base-pair with the upstream strand. The number of base pairs in a stem is the “length” of the stem. The tip of the stem is typically at least 3 nucleotides, but can be 4, 5, 6, 7, 8, 9, 10, 11, 12, 13, 14, 15 or more nucleotides. Larger tips with more than 5 nucleotides are also referred to as a “loop.” An otherwise continuous stem may be interrupted by one or more bulges as defined herein. The number of unpaired nucleotides in the bulge(s) are not included in the length of the stem. The position of a bulge closest to the tip can be described by the number of base pairs between the bulge and the tip (e.g., the bulge is 4 bps from the tip). The position of the other bulges (if any) further away from the tip can be described by the number of base pairs in the stem between the bulge in question and the tip, excluding any unpaired bases of other bulges in between.

### Nitro

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

### Non-Cationic Lipid

As used herein “non-cationic lipid” refers to any neutral, zwitterionic or anionic lipid.

### Open Reading Frame

An “open reading frame” is a continuous stretch of DNA beginning with a start codon (e.g., methionine (ATG)), and ending with a stop codon (e.g., TAA, TAG or TGA) and encodes a polypeptide.

### Orthologs

As used herein, “orthologs” refers to genes in different species that evolved from a common ancestral gene by speciation. Normally, orthologs retain the same function in the course of evolution. Identification of orthologs is critical for reliable prediction of gene function in newly sequenced genomes.

pegRNA

As used herein, the terms “prime editing guide RNA” or “pegRNA” or “PEgRNA” or “extended guide RNA” refer to a specialized form of a guide RNA that has been modified to include one or more additional sequences for implementing the prime editing methods and compositions described herein. As described herein, the prime editing guide RNA comprise one or more “extended regions” of nucleic acid sequence. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin). As used herein the “primer binding site” comprises a sequence that hybridizes to a single-strand DNA sequence having a 3′ end generated from the nicked DNA of the R-loop.

### PEI

As used herein, “PEI” refers to a PE complex comprising a fusion protein comprising Cas9(H840A) and a wild type MMLV RT having the following structure: [NLS]-[Cas9(H840A)]-[linker]-[MMLV_RT(wt)]+a desired pegRNA, wherein the PE fusion has the amino acid sequence of SEQ ID NO: 33. PE2
As used herein, “PE2” refers to a PE complex comprising a fusion protein comprising Cas9(H840A) and a variant MMLV RT having the following structure: [NLS]-[Cas9(H840A)]-[linker]-[MMLV_RT(D200N)(T330P)(L603W)(T306K)(W³¹³F)]+a desired pegRNA, wherein the PE fusion has the amino acid sequence of SEQ ID NO: 34.Pharmaceutically acceptable salt

The term “pharmaceutically acceptable salt” refers to a relatively non-toxic, inorganic or organic acid addition salt of a compound of the present disclosure which salt possesses the desired pharmacological activity.

### Pharmaceutically Acceptable Carrier, Diluent, or Excipient

As used herein, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Various aspects and embodiments are described in further detail in the following subsections.

### Pharmaceutical Composition

As used herein the term “pharmaceutical composition” refers to compositions comprising at least one active ingredient (e.g., an LNP encapsulated with a mRNA payload) and optionally one or more pharmaceutically acceptable excipients.

### PEG

As used herein “PEG” means any polyethylene glycol or other polyalkylene ether polymer.

### Peptide

As used herein, “peptide” is less than or equal to 50 amino acids long, e.g., about 5, 10, 15, 20, 25, 30, 35, 40, 45, or 50 amino acids long.

### PolyA Tail

A “polyA tail” is a region of mRNA that is downstream, e.g., directly downstream (i.e., 3′), from the 3 UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

### Polyamine

As used herein, “polyamine” means compounds having two or more amino groups. Examples include putrescine, cadaverine, spermidine, and spermine.

### Polypeptide Variant

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90% identity with a native or reference sequence.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity.

### Protein Fragment, Function Protein Domains, Homologous Proteins

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein.

### RNA

The term “RNA” is a well-known term of art that refers to ribonucleic acid.

### RNA-Guided Nuclease

As used herein, an “RNA-guided nuclease” is a type of “programmable nuclease,” and a specific type of “nucleic acid-guided nuclease.” As used herein, the term “RNA-guided nuclease” or “RNA-guided endonuclease” refers to a nuclease that associates covalently or non-covalently with a guide RNA thereby forming a complex between the guide RNA and the RNA-guided nuclease. The guide RNA comprises a spacer sequence which comprises a nucleotide sequence having complementarity with a strand of a target DNA sequence. Thus, the RNA-guided nuclease is indirectly guided or programmed to localize to a specific site in a DNA molecule through its association with the guide RNA, which directly binds or anneals to a strand of the target DNA through its complementarity region via Watson-Crick base-pairing.

### Sequence Identity

As used herein, the term “sequence identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). For example, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna. CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990).

### Spacer

As used herein the term “spacer” refers to a region of a polynucleotide or polypeptide ranging from 1 residue to hundreds or thousands of residues separating two other elements in a sequence. The sequence of the spacer can be defined or random. A spacer sequence is typically non-coding but may be a coding sequence.

### Structural Lipid

As used herein “structural lipid” refers to sterols and lipids containing sterol moieties.

### Subject

As used herein, the term“subject” refers to an individual organism, for example, an individual mammal or plant. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. The terms “individual,” “subject,” “host,” and “patient,” used interchangeably herein.

### Substituted

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, for example, 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂₀R^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)₀₋₂R^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{∘}₂, —NO₂, —SiR^{•}₃, —OSiR^{•}₃, —C(O)SR′, —(C₁₋₄ straight or branched alkylene)C(O)OR′, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{•}₂, ═NNHC(O)R^{•}, ═NNHC(O)OR^{•}, ═NNHS(O)₂R^{•}, ═NR^{•}, ═NOR^{•}, —O(C(R^{•}₂))₂₋₃O—, or —S(C(R^{•}₂))₂₋₃S—, wherein each independent occurrence of R^{•}is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{•}₂)₂₋₃O—, wherein each independent occurrence of R^{•}is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{•} include halogen, —R^{•}, -(haloR^{∘}), —OH, —OR^{•}, —O(haloR^{•}), —CN, —C(O)OH, —C(O)OR′, —NH₂, —NHR^{•}, —NR′₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)ORT, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R′ are independently halogen, —R^{•}, -(haloR^{•}), —OH, —OR^{•}, —O(haloR^{∘}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation, for example, by rearrangement, cyclization, or elimination.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

### Substantially

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### Target or Target Nucleic Acid

A “target” or “target nucleic acid” as used herein is a polynucleotide (e.g., DNA such as genomic DNA) that includes a site (“target site” or “target sequence”) targeted by a nucleic acid programmable DNA binding protein of the present disclosure. The target sequence is the sequence to which the guide sequence of a subject nucleic acid programmable DNA binding protein will hybridize. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

### Terminus

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

### Treating

The terms “treat,” “treating,” and “treatment,” as used herein, refer to therapeutic or preventative measures such as those described herein.

### Unmodified

As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Upstream and Downstream

As used herein, the terms “upstream” and “downstream” are terms of relativity that define the linear position of at least two elements located in a nucleic acid molecule (whether single or double-stranded) that is orientated in a 5′-to-3′ direction. A first element is said to be upstream of a second element in a nucleic acid molecule where the first element is positioned somewhere that is 5′ to the second element. Conversely, a first element is downstream of a second element in a nucleic acid molecule where the first element is positioned somewhere that is 3′ to the second element.

### Vaccine

As used herein, the phrase “vaccine” refers to a biological preparation that improves immunity in the context of a particular disease, disorder or condition.

### Vector

As used herein, a “vector” is any molecule or moiety which transports, transduces or otherwise acts as a carrier of a heterologous molecule. Vectors of the present disclosure may be produced recombinantly and may be based on and/or may comprise viral parent or reference sequence. Such parent or reference viral sequences may serve as an original, second, third or subsequent sequence for engineering vectors. In non-limiting examples, such parent or reference viral sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These viral sequences may serve as either the “donor” sequence of one or more codons (at the nucleic acid level) or amino acids (at the polypeptide level) or “acceptor” sequences of one or more codons (at the nucleic acid level) or amino acids (at the polypeptide level). The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### 3′ Untranslated Region

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

### 5′ Untranslated Region

A “5′ untranslated region” (UTR) refers to a region of an mRNA that is directly upstream (i.e., 5′) from the start codon (i.e., the first codon of an mRNA transcript translated by a ribosome) that does not encode a polypeptide.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

As used herein, the following abbreviations and initialisms have the indicated meanings:

| | |
|---|---|
| MC3 | 4-(dimethylamino)-butanoic acid, (10Z,13Z)-1-(9Z,12Z)-9,12-octadecadien-1- |
| | y1-10,13-nonadecadien-1-yl ester |
| DSPC | 1,2-distearoyl-sn-glycero-3-phosphocholine |
| DMG | 1,2-Dimyristoyl-rac-glycero-3-methanol |
| DOMG-PEG | R-3-[(ω-methoxy-poly(ethyleneglycol))carbamoyl)]-1,2-dimyristyloxypropyl- |
| | 3-amine |
| DLPE | 1,2-Dilauroyl-sn-Glycero-3-Phosphoethanolamine |
| DMPE | 1,2-Dimyristoyl-sn-Glycero-3-Phosphoethanolamine |
| DPPC | 1,2-dipalmitoyl-sn-glycero-3-phosphocholine |
| DSPE | 1,2-distearoyl-sn-glycero-3-phosphoethanolamine |
| DDAB | Didodecyldimethylammonium bromide |
| EPC | 1,2-dioleoyl-sn-glycero-3-ethylphosphocholine |
| 14PA | 1,2-dimyristoyl-sn-glycero-3-phosphate |
| 18BMP | bis(monooleoylglycero)phosphate |
| DODAP | 1,2-dioleoyl-3-dimethylammonium-propane |
| DOTAP | 1,2-dioleoy1-3-trimethylammonium-propane |
| C12-200 | 1,1′-((2-(4-(2-((2-(bis(2-hydroxydodecyl)amino)ethyl)(2- |
| | hydroxydodecyl)amino)ethyl)piperazin-1-yl)ethyl)azanediyl)bis(dodecan-2-ol) |

### Enumerated Embodiments

The present disclosure includes embodiments 1-21:
1. A pharmaceutical composition comprising:
   a) at least one lipid nanoparticle comprising:
      i) at least one ionizable lipid selected from those listed in Tables (I), (11) and (III); or
      ii) at least one ionizable lipid of a Formula selected from Formulas VIJ-A, VIII-A, IX-A, VII-B, VII-C, I-A, II, III-B, III-C, III-D, III-E, III-F, VIII-B, IV, VI, X, X-A, CY, CY-I, CY-II, CY-III, CY-IV, CY-V, CY-VI, CY-VII, CY-VII, CY-IX, CY-IVa, CY-IVb, CY-IVc, CY-IVd, CY-IVe, CY-IVf, CY-I′, CY-II′, CY-II′, CY-IV′, CY-V′, CY-VI′, IA, IB, IC, ID, I, II, III, IV, V, VI, VI′, VI″, VII, VII′, VII″, XV, XV′, XV″, XV′, XVI, XVI′, XVI″, XVI′, XVII, XVIII, XVIII′, XVIX, XIX, XX, and XXI; and
   b) at least one nucleobase editing system.
2. The pharmaceutical composition of embodiment 1, wherein the nucleobase editing system comprises a CRISPR-Cas gene editing system.
3. The pharmaceutical composition of embodiment 1, wherein the nucleobase editing system comprises a prime editing system or components thereof.
4. The pharmaceutical composition of embodiment 1, wherein the nucleobase editing system comprises a retron editing system.
5. The pharmaceutical composition of embodiment 1, wherein the nucleobase editing system comprises a TnpB editing system.
6. The pharmaceutical composition of embodiment 1, wherein the nucleobase editing system comprises an integrase editing system.
7. The pharmaceutical composition of embodiment 1, wherein the nucleobase editing system comprises a base editing system.
8. The pharmaceutical composition of embodiment 1, wherein the nucleobase editing system comprises an epigenetic editing system.
9. The pharmaceutical composition of embodiment 1, wherein the nucleobase editing system comprises a gene writing system.
10. The pharmaceutical composition of embodiment 1, wherein the nucleobase editing system comprises a gene inactivating system.
11. The pharmaceutical composition of embodiment 1, wherein the nucleobase editing system comprises zinc finger nuclease.
12. The pharmaceutical composition of embodiment 1, wherein the nucleobase editing system comprises a TALE Nuclease, a TALE nickase, Zinc Finger (ZF) Nuclease, ZF Nickase, meganuclease, or a combination thereof.
13. The pharmaceutical composition of embodiment 1, wherein the nucleobase editing system comprises a meganuclease.
14. The pharmaceutical composition of any one of embodiments 1-13, wherein the at least one lipid nanoparticle further comprises:
   i) at least one structural lipid;
   ii) at least one phospholipid; and
   iii) at least one PEGylated lipid.
15. The pharmaceutical composition of any one of embodiments 1-14, wherein the at least one structural lipid is selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and any combinations thereof.
16. The pharmaceutical composition of any one of embodiments 1-15, wherein the at least one phospholipid is selected from 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium (S)-2-ammonio-3-((((R)-2-(oleoyloxy)-3-(stearoyloxy)propoxy)oxidophosphoryl)oxy)propanoate (L-a-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin.
17. The pharmaceutical composition of any one of embodiments 1-16, wherein the at least one PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.
18. The pharmaceutical composition of any one of embodiments 1-17, wherein the LNP further comprises at least one additional lipid component selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-rihydroxy-6-hydroxymethyl-letrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-a-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g.1,2-dioleoyl-sn-glycero-3-phosphoethanolamine-N-[4-(p-maleimidophenyl)but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol(PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.
19. A method of delivering a nucleobase editing system to a subject in need thereof, the method comprising administering to the subject the pharmaceutical composition of any one of embodiments 1-18.
20. The pharmaceutical composition of any of embodiments 1-19 for use as a medicament.
21. Use of a pharmaceutical composition of any of embodiments 1-20 for the manufacture of a medicament for delivery of a nucleobase editing system.

The present disclosure includes embodiments 1A-21A:
1A. A pharmaceutical composition comprising:
   a) at least one lipid nanoparticle comprising:
      i) at least one ionizable lipid selected from those listed in Tables (I), (II), (III); (IV), or (V)
      ii) at least one ionizable lipid of a Formula selected from Formulas VII-A, VIII-A, IX-A, VII-B, VII-C, I-A, II, III-B, III-C, III-D, III-E, III-F, VIII-B, IV, VI, X, X-A, CX-I, CX-i, CY, CY-I, CY-II, CY-III, CY-IV, CY-V, CY-VI, CY-VII, CY-VIII, CY-IX, CY-IVa, CY-IVb, CY-IVc, CY-IVd, CY-IVe, CY-IVf, CY-I′, CY-II′, CY-III′, CY-IV′, CY-V′, CY-VI′, CZ-I, IA, IB, IC, ID, I, II, III, IV, V, VI, VI′, VI″, VII, VII′, VII″, VII′″, VIII, VIII′, VIII″, VIII′″, IX, IX′, IX″, IX′″, X, X′, X″, X′″, XI, XI′, XI″, XI′″, XII, XII′, XII″, XII′″, XIII, XIII′, XIII″, XIII′″, XIV, XIV′, XIV″, XIV′″, XV, XV′, XV″, XV′″, XVI, XVI′, XVI″, XVI′″, XVII, XVIII, XVIII′, XVIX, XIX, XX, and XXI; and
   b) at least one nucleobase editing system.
2A. The pharmaceutical composition of embodiment 1 A, wherein the nucleobase editing system comprises a CRISPR-Cas gene editing system.
3A. The pharmaceutical composition of claim 2A, wherein the nucleobase editing system comprises a Type V CRISPR-Cas gene editing system.
4A. The pharmaceutical composition of embodiment 1A, wherein the nucleobase editing system comprises a prime editing system or components thereof.
5A. The pharmaceutical composition of embodiment IA, wherein the nucleobase editing system comprises a retron editing system.
6A. The pharmaceutical composition of embodiment TA, wherein the nucleobase editing system comprises a TnpB editing system.
7A. The pharmaceutical composition of embodiment TA, wherein the nucleobase editing system comprises an integrase editing system.
8A. The pharmaceutical composition of embodiment IA, wherein the nucleobase editing system comprises a base editing system.
9A. The pharmaceutical composition of embodiment TA, wherein the nucleobase editing system comprises an epigenetic editing system.
10A. The pharmaceutical composition of embodiment TA, wherein the nucleobase editing system comprises a gene writing system.
11A. The pharmaceutical composition of embodiment IA, wherein the nucleobase editing system comprises a gene inactivating system.
12A. The pharmaceutical composition of embodiment TA, wherein the nucleobase editing system comprises zinc finger nuclease.
13A. The pharmaceutical composition of embodiment TA, wherein the nucleobase editing system comprises a TALE Nuclease, a TALE nickase, Zinc Finger (ZF) Nuclease, ZF Nickase, meganuclease, or a combination thereof.
14A. The pharmaceutical composition of embodiment TA, wherein the nucleobase editing system comprises a meganuclease.
15A. The pharmaceutical composition of any one of embodiments 1A-14A, wherein the at least one lipid nanoparticle further comprises:
   i) at least one structural lipid;
   ii) at least one phospholipid; and
   iii) at least one PEGylated lipid.
16A. The pharmaceutical composition of any one of embodiments 1A-15A, wherein the at least one structural lipid is selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and any combinations thereof.
17A. The pharmaceutical composition of any one of embodiments 1A-16A, wherein the at least one phospholipid is selected from 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium (S)-2-ammonio-3-((((R)-2-(oleoyloxy)-3-(stearoyloxy)propoxy)oxidophosphoryl)oxy)propanoate (L-a-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin.
18A. The pharmaceutical composition of any one of embodiments 1A-17A, wherein the at least one PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.
19A. The pharmaceutical composition of any one of embodiments 1A-18A, wherein the LNP further comprises at least one additional lipid component selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4(Man), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-a-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide](MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g.1,2-dioleoyl-sn-glycero-3-phosphoethanolamine-N-[4-(p-maleimidophenyl)but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol(PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.
20A. A method of delivering a nucleobase editing system to a subject in need thereof, the method comprising administering to the subject the pharmaceutical composition of any one of embodiments 1A-19A.
21A. The pharmaceutical composition of any of embodiments 1A-19A for use as a medicament.
22A. Use of a pharmaceutical composition of any of embodiments 1A-19A for the manufacture of a medicament for delivery of a nucleobase editing system.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The following examples represent exemplary aspects of the present disclosure.

### Example 1: Synthesis of Exemplary Ionizable Lipids

Exemplary ionizable lipids of the present disclosure, as shown in Tables (I) and (II), can be made by methods described in PCT/US2022/076430 and PCT/US2022/076415, both of which are incorporated by reference herein, in their entirety.

### Synthesis of Compound 11 (Bis(3-pentyloctyl) 8,8′-((4-hydroxybutyl)azanediyl) dioctanoate)

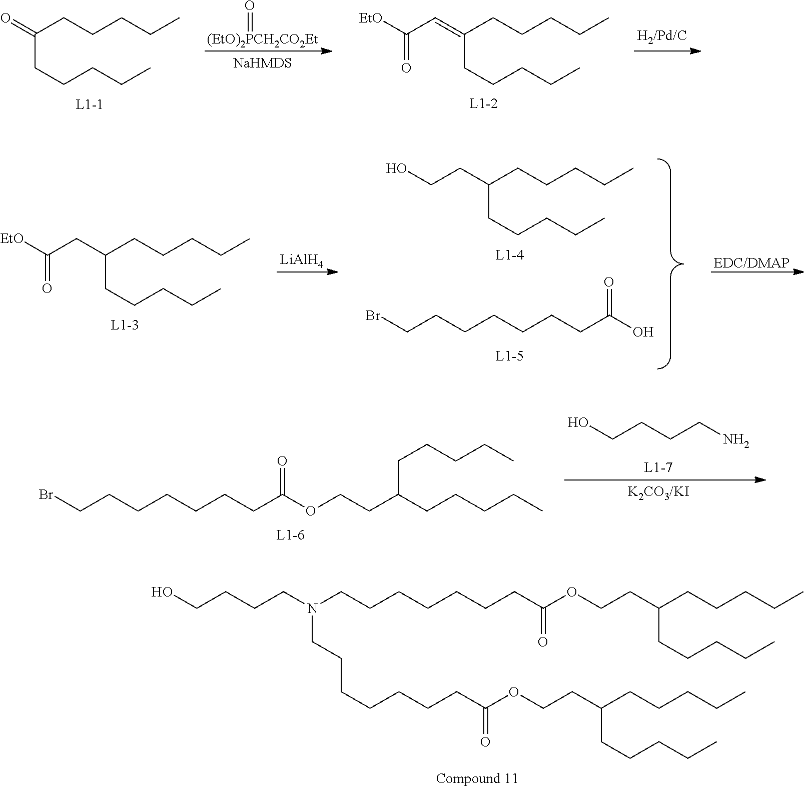

### Synthesis of ethyl 3-pentyloct-2-enoate (L1-2)

To a solution of triethyl phophonoacetate (26.3 g, 118 mmol) in anhydrous THF (33 mL) was added 1M NaHMDS in THF (118 mL, 118 mmol), dropwise, at −10 to −15° C. under nitrogen atmosphere. After completion of the addition, the mixture was stirred at the same temperature for 30 min and then at 0° C. for 1 h. To this mixture was dropped in 6-undecanone (10.0 g, 59 mmol) at 0° C. and the reaction mixture was allowed to warm to room temperature and stirred overnight. It was then warmed to 45° C. and stirred for an additional 24h. Aq. sat. NH₄Cl (8 mL) was added and the THF was evaporated. The residue was mixed with Et₂O (80 mL) and H₂O (100 mL) and the resulting phases were separated. The aqueous phase was extracted with Et₂O (80 mL). Combined organic phases were washed with H₂O (100 mL×2) and dried over anhydrous Na₂SO₄. Filtration and concentration provided crude material which was purified by flash column chromatography (SiO₂: 0 to 4% ethyl acetate in hexane gradient) to yield ethyl 3-pentyloct-2-enoate L¹-2 as colorless oil (11.6 g, 82%). ¹H-NMR (300 MHz, CDCl₃) δ 5.60 (s, 1H), 4.13 (q, J=7.1 Hz, 2H), 2.57 (t, J=7.6 Hz, 2H), 2.12 (t, J=7.4 Hz, 2H), 1.52-1.19 (m, 15H), 0.89 (t, J=7.2 Hz, 6H); CIMS m/z 241 [M+H]⁺.

### Synthesis of ethyl 3-pentyloctanoate (L1-3)

To a solution of L1-2 (11.0 g, 2.1 mmol) in EtOAc (90 mL) was added 10% Pd/C (0.5 g). The resulting mixture was stirred under a hydrogen balloon for one day. It was then filtered through Celite. The Celite was rinsed with EtOAc (25 mL×3). The combined filtrate was evaporated to give ethyl 3-pentyloctanoate L1-3 as a light-yellow oil (9.0 g, 83%). ¹H-NMR (300 MHz, CDCl₃) δ 4.10 (q, J=7.1 Hz, 2H), 2.20 (d, J=6.8 Hz, 2H), 1.82 (s, 1H), 1.40-1.12 (m, 19H), 0.88 (t, J=7.0 Hz, 6H).

### Synthesis of 3-pentyloctan-1-ol (L1-4)

To a 2.0 M THF solution of lithium aluminum hydride (28 mL, 56 mmol) was added slowly a solution of L1-3 (7.0 g, 29 mmol) in THF (33 mL) at 0° C. under nitrogen atmosphere. The resulting mixture was stirred at 0° C. for 1 h then at room temperature overnight. With ice-water bath cooling, the reaction was quenched by adding saturated aqueous Na₂SO₄ solution to give a milky solution. The organic phase was separated, and the aqueous phase was extracted with Et₂O (50 mL×2). The combined organic phases were dried over Na₂SO₄.Filtration and concentration provided crude material which was purified by flash column chromatography (SiO₂: 0 to 15% ethyl acetate in hexane gradient) to yield 3-pentyloctan-1-ol L1-4 as slightly yellow oil (4.0 g, 70%). ¹H-NMR (300 MHz, CDCl₃) 3.65 (t, J=4.4 Hz, 2H), 1.51 (dd, J=13.7 Hz, 6.8 Hz, 2H), 1.46-1.12 (m, 17H), 0.88 (t, J=7.1 Hz, 6H).

### Synthesis of 3-pentyloctyl 8-bromooctanoate (L1-6)

To a solution of L1-4 (0.8 g, 4 mmol) in DCM (10 mL) was added 8-bromooctanoic acid L1-5 (1.0 g, 4.4 mmol) followed by DMAP (0.3 g, 2 mmol) and EDC (1.9 g, 8 mmol). The resulting mixture was stirred at room temperature under nitrogen atmosphere overnight. The reaction mixture was diluted with DCM (10 mL) and washed with saturated NaHCO₃ aqueous solution (10 mL), water (10 mL) and brine (10 mL). The organic phase was dried over anhydrous Na₂SO₄. Filtration and concentration provided crude material which was purified by flash column chromatography (SiO₂: 0 to 10% ethyl acetate in hexane gradient) to yield 3-pentyloctyl 8-bromooctanoate L1-6 as colorless oil (1.0 g, 78%). ¹H-NMR (300 MHz, CDCl₃) δ 4.07 (t, J=7.3 Hz, 2H), 3.39 (t, J=6.9 Hz, 2H), 2.28 (t, J=7.7 Hz, 2H), 1.90-1.15 (m, 29H), 0.88 (t, J=6.9 Hz, 6H).

### Synthesis of bis(3-pentyloctyl) 8,8′-((4-hydroxybutyl)azanediyl) dioctanoate (Compound 11)

A solution of L1-6 (650 mg, 1.6 mmol), 4-amino-1-butanol L1-7 (64 mg, 0.7 mmol) in cyclopentyl methyl ether (10 mL) and acetonitrile (10 mL) containing potassium carbonate (400 mg, 2.8 mmol) and potassium iodide (120 mg, 0.7 mmol) was heated at 85° C. for 24 hours. After cooling to room temperature, the reaction mixture was filtered through Celite, washed with ethyl acetate, and then the solvent was removed under vacuum to give the crude product which was purified by flash chromatography (SiO₂: ethyl acetate/hexane 0-100% with 1% triethylamine in the eluent) to get Compound 11 (337 mg, 63%) as slightly yellow oil. ¹H NMR (300 MHz, CDCl₃): δ ppm 4.06 (t, J=7.0 Hz, 4H), 3.52 (m, 2H), 2.40 (m, 6H), 2.26 (t, J=7.7 Hz, 4H), 1.70-1.15 (m, 68H), 0.86 (t, J=7.1 Hz, 12H); MS (CI): m/z [M+H]⁺ 738.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: 99.61%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=13.1 min, purity: 97.30%.

### Synthesis of heptadecan-9-yl 8-((2-((2-hydroxyethyl)(methyl)amino)ethyl)(6-oxo-6-(undecyloxy)hexyl)amino)octanoate (Compound 46)

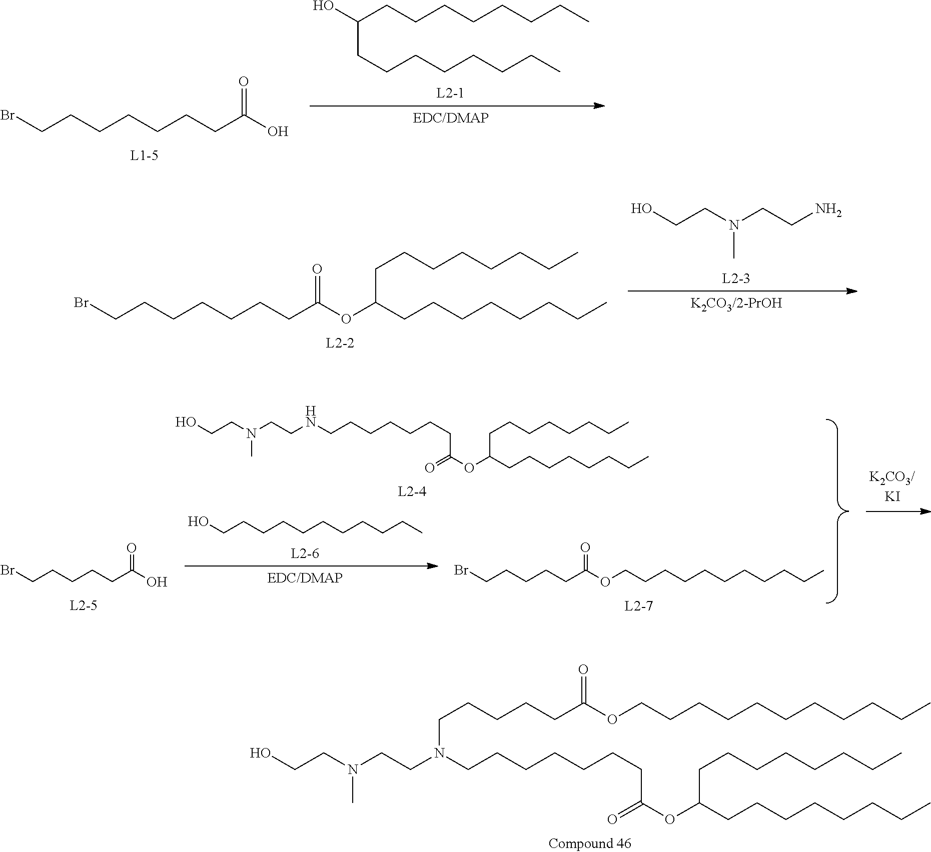

### Synthesis of heptadecan-9-yl 8-bromooctanoate (L2-2)

To a solution of L2-1 (2.56 g, 1 mmol) in DCM (60 mL) was added 8-bromooctanoic acid L1-5 (2.22 g, 1 mmol) followed by DMAP (0.61 g, 0.5 mmol) and EDC (3.9 g, 2 mmol). The resulting mixture was stirred at room temperature under nitrogen atmosphere overnight. The reaction mixture was diluted with DCM (50 mL) and washed with saturated NaHCO₃ aqueous solution (50 mL), water (30 mL) and brine (30 mL). The organic phase was dried over anhydrous Na₂SO₄. Filtration and concentration provided crude material which was purified by flash column chromatography (SiO₂: 0 to 10% ethyl acetate in hexane gradient) to yield 3-pentyloctyl 8-bromooctanoate L²-2 as colorless oil (3.2 g, 70%). ¹H-NMR (300 MHz, CDCl₃) δ 4.85 (quintet, J=6.1 Hz, 1H), 3.40 (t, J=6.9 Hz, 2H), 2.28 (t, J=7.7 Hz, 2H), 1.86 (quintet, J=6.0 Hz, 2H), 1.70-1.15 (m, 36H), 0.87 (t, J=6.9 Hz, 6H).

### Synthesis of heptadecan-9-yl 8-((2-((2-hydroxyethyl)(methyl)amino)ethyl)amino)octanoate (L2-4) and di(heptadecan-9-yl) 8,8′-((2-((2-hydroxyethyl)(methyl)amino)ethyl)azanediyl)dioctanoate (Compound 47)

A mixture of L2-2 (1.0 g, 2.2 mmol), and 2-[(2-aminoethyl)(methyl)amino]ethanol L²-3 (1.28 g, 11 mmol) in 2-propanol (10 mL) containing potassium carbonate (0.28 g, 2.0 mmol) was heated at 55-60° C. for 3.5 days. After cooling to room temperature, the reaction mixture was filtered through Celite. Concentration gave an oil residue which was purified by flash chromatography (SiO₂: ethyl acetate/hexane 0-100% with 1-30% triethylamine in the eluent) to give L²-4 (0.79 g, 72%) as slightly yellow oil. ¹H NMR (300 MHz, CDCl₃): δ ppm 4.85 (quintet, J=6.1 Hz, 1H), 3.59 (t, J=5.2 Hz, 2H), 2.70 (t, J=6.1 Hz, 2H), 2.62-2.48 (m, 6H), 2.29 (s, 3H), 2.27 (t, J=7.7 Hz, 2H), 1.73-1.16 (m, 40H), 0.87 (t, J=6.6 Hz, 6H); MS (CI): m/z [M+H]⁺ 499; The bis-addition compound product (Compound 47) (69 mg) was also isolated as slightly yellow oil. ¹H NMR (300 MHz, CDCl₃): δ ppm 4.86 (quintet, J=6.3 Hz, 1H), 3.55 (t, J=4.9 Hz, 2H), 2.54 (t, J=5.4 Hz, 2H), 2.49 (s, 4H), 2.40 (t, J=7.4 Hz, 4H), 2.30 (s, 3H), 2.27 (t, J=7.6 Hz, 4H), 1.73-1.16 (m, 84H), 0.87 (t, J=6.6 Hz, 6H); MS (CI): m/z [M+H]⁺ 879.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=12.0 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=14.8 min, purity: 99.42%.

### Synthesis of undecyl 6-bromohexanoate (L2-7)

To a solution of L2-6 (5.16 g, 30 mmol) in DCM (160 mL) was added 6-bromohexanoic acid L²-5 (5.85 g, 30 mmol) followed by DMAP (1.8 g, 15 mmol) and EDC (11.9 g, 60 mmol). The resulting mixture was stirred at room temperature under nitrogen atmosphere for 20 h. The reaction mixture was diluted with DCM (150 mL) and washed with saturated NaHCO₃ aqueous solution (150 mL), water (100 mL) and brine (100 mL). The organic phase was dried over anhydrous Na₂SO₄. Filtration and concentration provided crude material which was purified by flash column chromatography (SiO₂: 0 to 10% ethyl acetate in hexane gradient) to yield undecyl 6-bromohexanoate L²-7 as slightly yellow oil (8.5 g, 81%). ¹H-NMR (300 MHz, CDCl₃) δ 4.06 (t, J=6.9 Hz, 2H), 3.40 (t, J=6.6 Hz, 2H), 2.31 (t, J=7.4 Hz, 2H), 1.88 (quintet, J=6.1 Hz, 2H), 1.70-1.15 (m, 19H), 0.88 (t, J=6.9 Hz, 6H).

### Synthesis of Compound 46 ((heptadecan-9-yl 8-((2-((2-hydroxyethyl)(methyl)amino)ethyl)(6-oxo-6-(undecyloxy)hexyl)amino)octanoate)

A mixture of L2-4 (0.4 g, 0.8 mmol) and L2-7 (0.4 g, 1.2 mmol) in cyclopentyl methyl ether (5 mL) and acetonitrile (5 mL) containing potassium carbonate (0.33 g, 2.4 mmol) and potassium iodide (0.134 g, 0.8 mmol) was heated at 80° C. under nitrogen with stirring for one day. After cooling to room temperature, the reaction mixture was filtered through Celite. The filtrate was concentrated to give an oil residue which was purified by flash chromatography (SiO₂: ethyl acetate/hexane 0-100% with 1% triethylamine in the eluent) to yield Compound 46 (0.319 g, 52%) as slightly yellow oil. ¹H NMR (300 MHz, CDCl₃): δ ppm 4.85 (quintet, J=6.3 Hz, 1H), 4.04 (t, J=6.6 Hz, 2H), 3.55 (t, J=5.2 Hz, 2H), 2.53 (t, J=5.0 Hz, 2H), 2.49 (s, 4H), 2.40 (m, 4H), 2.30 (s, 3H), 2.27 (t, J=7.4 Hz, 4H), 1.73-1.16 (m, 66H), 0.87 (t, J=6.6 Hz, 6H); MS (CI): m/z [M+H]⁺ 767.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.1 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, A in B 60% to 100% in 15 min, flow rate: 0.5 mL/min, column temperature: 20±2° C., detector: CAD, t_{R}=12.4 min, purity: 98.95%.

### Synthesis of Compound 9 (2-Isopropyl-5-methylhexyl 8-((4-hydroxybutyl)(6-((2-isopropyl-5-methylhexyl)oxy)-6-oxohexyl)amino)octanoate)

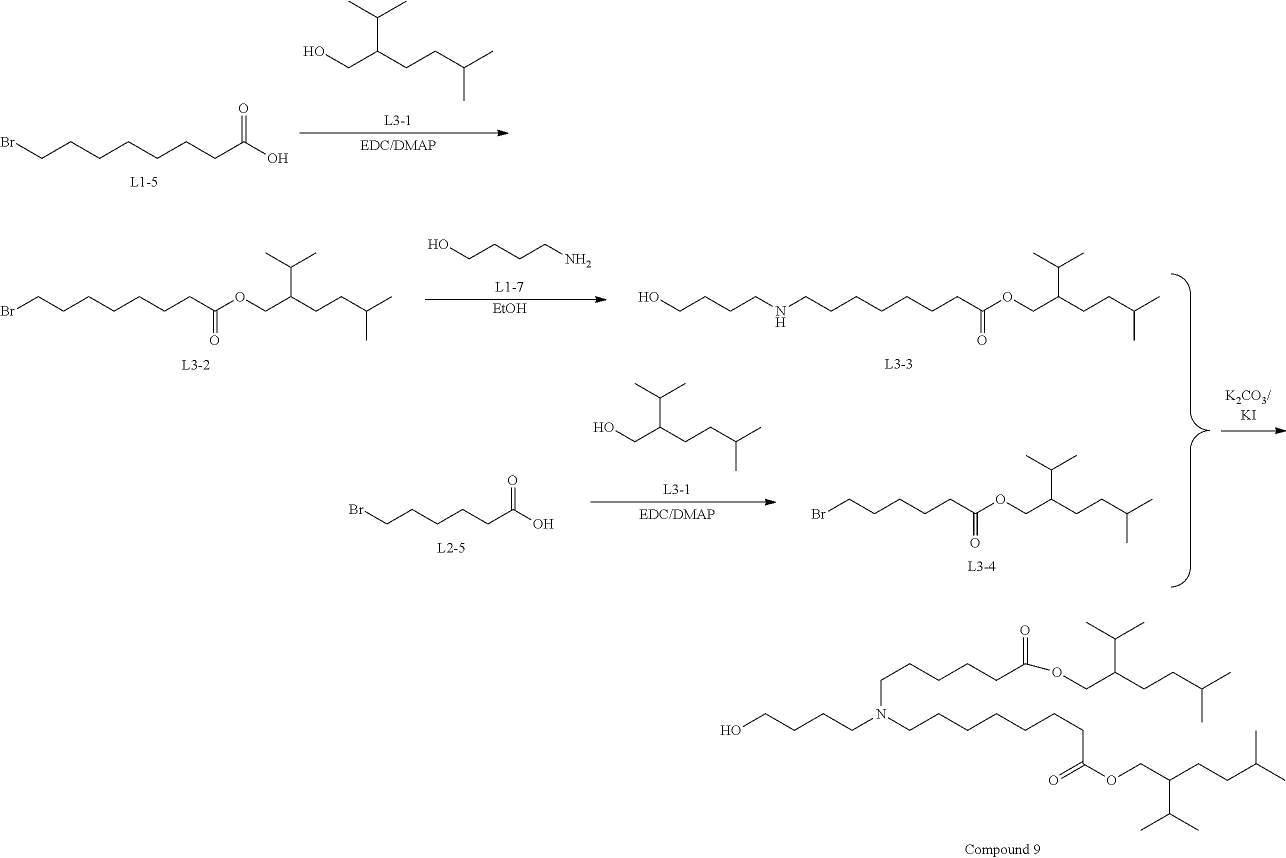

### Synthesis of 2-isopropyl-5-methylhexyl 8-bromooctanoate (L3-2)

To a solution of tetrahydrolavandulol L3-1 (1.2 g, 7.6 mmol) in DCM (40 mL) was added 8-bromooctanoic acid L1-5 (1.69 g, 7.6 mmol) followed by DMAP (0.46 g, 3.8 mmol) and EDC (2.9 g, 15.2 mmol). The resulting mixture was stirred at room temperature under nitrogen atmosphere for 20 h. The reaction mixture was diluted with DCM (40 mL) and washed with saturated NaHCO₃ aqueous solution (40 mL), water (30 mL) and brine (30 mL). The organic phase was dried over anhydrous Na₂SO₄. Filtration and concentration provided crude material which was purified by flash column chromatography (SiO₂: 0 to 10% ethyl acetate in hexane gradient) to yield 2-isopropyl-5-methylhexyl 8-bromooctanoate L3-2 as colorless oil (1.8 g, 65%). ¹H-NMR (300 MHz, CDCl₃) δ 4.10-3.91 (m, 2H), 3.40 (t, J=6.6 Hz, 2H), 2.29 (t, J=7.6 Hz, 2H), 1.90-1.10 (m, 17H), 0.93-0.76 (m, 12H).

### Synthesis of Compound 48 (2-isopropyl-5-methylhexyl 8-((4-hydroxybutyl)amino)octanoate (L3-3) and bis(2-isopropyl-5-methylhexyl) 8,8′-((4-hydroxybutyl)azanediyl)dioctanoate)

A mixture of L3-2 (1.2 g, 3.3 mmol), 4-amino-1-butanol L1-7 (1.47 g, 16.5 mmol) in abs. EtOH (10 mL) containing potassium carbonate (0.38 g, 3.3 mmol) was heated at 55° C. for 3.5 days. After cooling to room temperature, the reaction mixture was filtered through Celite. The filtrate was concentrated to give an oil residue which was purified by flash chromatography (SiO₂: ethyl acetate/hexane 0-100% with 1-30% triethylamine in the eluent) to yield L3-3 (0.67 g, 54%) as slightly yellow oil. ¹H NMR (300 MHz, CDCl₃): δ ppm 4.86 (quintet, J=6.3 Hz, 1H), 3.55 (t, J=4.9 Hz, 2H), 2.54 (t, J=5.4 Hz, 2H), 2.49 (s, 4H), 2.40 (t, J=7.4 Hz, 4H), 2.30 (s, 3H), 2.27 (t, J=7.6 Hz, 4H), 1.73-1.16 (m, 84H), 0.87 (t, J=6.6 Hz, 6H); MS (CI): m/z [M+H]⁺ 373. The bis-addition product (Compound 48) (62 mg) was also isolated as slightly yellow oil. ¹H NMR (300 MHz, CDCl₃): δ ppm 4.86 (quintet, J=6.3 Hz, 1H), 3.55 (t, J=4.9 Hz, 2H), 2.54 (t, J=5.4 Hz, 2H), 2.49 (s, 4H), 2.40 (t, J=7.4 Hz, 4H), 2.30 (s, 3H), 2.27 (t, J=7.6 Hz, 4H), 1.73-1.16 (m, 84H), 0.87 (t, J=6.6 Hz, 6H); MS (CI): m/z [M+H]⁺ 654.5; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=5.42 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=9.8 min, purity: 97.20%.

### Synthesis of 2-isopropyl-5-methylhexyl 6-bromohexanoate (L3-4)

To a solution of tetrahydrolavandulol L3-1 (0.8 g, 5 mmol) in DCM (25 mL) was added 6-bromohexanoic acid L2-5 (0.98 g, 5 mmol) followed by DMAP (0.3 g, 2.5 mmol) and EDC (1.7 g, 9 mmol). The resulting mixture was stirred at room temperature under nitrogen atmosphere for 20 h. The reaction mixture was diluted with DCM (25 mL) and washed with saturated NaHCO₃ aqueous solution (25 mL), water (15 mL) and brine (15 mL). The organic phase was dried over anhydrous Na₂SO₄. Filtration and concentration provided crude material which was purified by flash column chromatography (SiO₂: 0 to 10% ethyl acetate in hexane gradient) to yield L3-4 as colorless oil (0.76 g, 45%). ¹H-NMR (300 MHz, CDCl₃) δ 4.10-3.92 (m, 2H), 3.40 (t, J=6.6 Hz, 2H), 2.32 (t, J=7.4 Hz, 2H), 1.92-1.10 (m, 13H), 0.93-0.76 (m, 12H).

### Synthesis of Compound 9 (2-isopropyl-5-methylhexyl 8-((4-hydroxybutyl)(6-((2-isopropyl-5-methylhexyl)oxy)-6-oxohexyl)amino)octanoate)

A mixture of L3-3 (0.3 g, 0.8 mmol) and L3-4 (0.3 g, 0.9 mmol) in cyclopentyl methyl ether (5 mL) and acetonitrile (5 mL) containing potassium carbonate (0.19 g, 2.4 mmol) and potassium iodide (0.134 g, 0.8 mmol) was heated at 80° C. under nitrogen with stirring for one day. More of L3-4 (0.2 g, 0.6 mmol) was added and the reaction continued for another two days. After cooling to room temperature, the reaction mixture was filtered through Celite. The filtrate was concentrated to give an oil residue which was purified by flash chromatography (SiO₂: ethyl acetate/hexane 0-100% with 1% triethylamine in the eluent) to yield Compound 9 (0.276 g, 55%) as slightly yellow oil. ¹H NMR (300 MHz, CDCl₃): δ ppm 4.10-3.91 (m, 4H), 3.53 (t, J=5.2 Hz, 2H), 2.49-2.22 (m, 10H), 1.82-1.12 (m, 35H), 0.93-0.79 (m, 24H); MS (CI): m/z [M+H]⁺ 626.5; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=5.8 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=8.8 min, purity: 98.29%.

### Synthesis of Compound 14 ((4-hydroxybutyl)azanediyl)bis(hexane-6,1-diyl) bis(4,4-bis(octyloxy)butanoate)

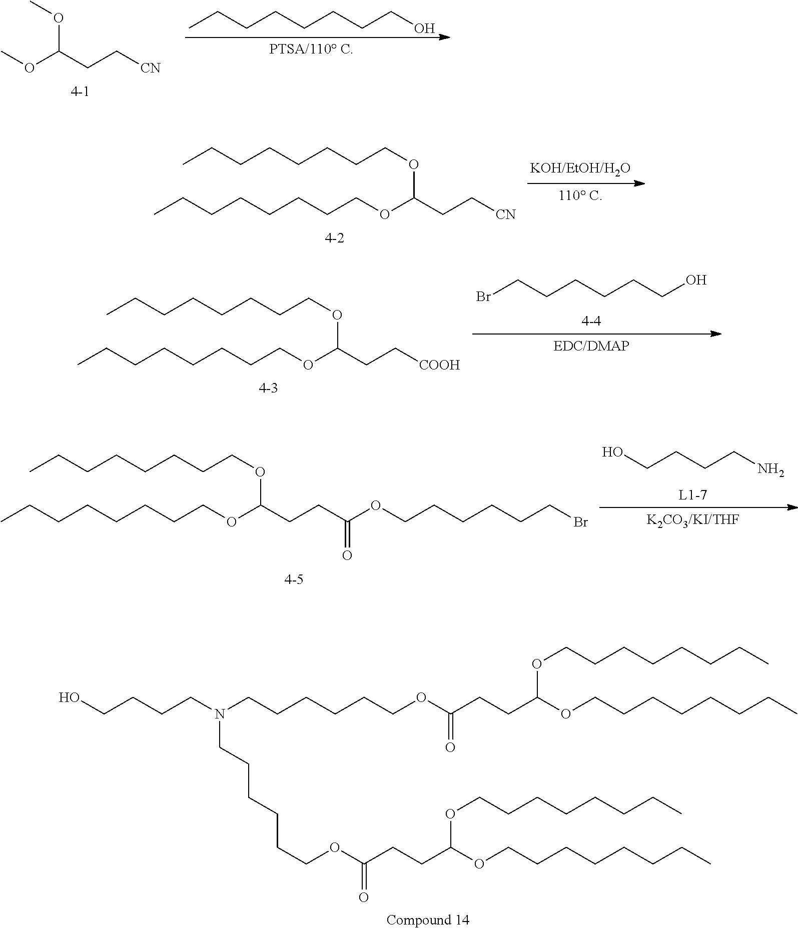

### Synthesis of 4,4-bis(octyloxy)butanenitrile (L4-2)

To an oven dried 100 mL round bottom flask, 4,4-dimethoxybutanenitrile (10.0 g, 77.4 mmol) and 1-octanol (24.9 g, 232 mmol) were added. The resulting mixture was stirred at 120° C. for 18 h and cooled to room temperature. EtOAc (150 mL) and H₂O (60 mL) were added in, and the resulting phases were separated. The aqueous phase was extracted with EtOAc (50 mL). Combined organic extracts were washed with H₂O (60 mL×2) and dried over anhydrous MgSO₄. Filtration and concentration provided crude material which was purified by flash column chromatography (SiO₂: 0 to 10% ethyl acetate in hexanes gradient) to yield 4,4-bis(octyloxy)butanenitrile L4-2 as colorless oil (6.9 g, 27%). ¹H-NMR (300 MHz, CDCl₃) δ 4.56-4.53 (t, 1H), 3.61-3.58 (2H, m), 3.44-3.41 (m, 2H), 2.44-2.39 (t, 2H), 1.97-1.90 (q, 2H), 1.58-1.54 (m, 5H), 1.32-1.27 (m, 23H), 0.90-0.85 (m, 6H).

### Synthesis of 4,4-bis(octyloxy)butanoic acid (L4-3)

To an oven dried 100 mL round bottom flask containing a solution of 4,4-bis(octyloxy)butanenitrile L4-2 (6.9 g, 21 mmol) in ethanol (25 mL) was added a solution of KOH (3.6 g, 64 mmol) in water (25 mL). After completion of the addition, the mixture was stirred at 110° C. for 18 h. The volatiles were removed, and the reaction pH was adjusted to 5. EtOAc (150 mL) and H₂O (60 mL) were added, and the resulting phases were separated. The aqueous phase was extracted with EtOAc (50 mL). Combined organic extracts were washed with H₂O (60 mL×2) and dried over anhydrous MgSO₄. Filtration and concentration provided 4,4-bis(octyloxy)butanoic acid L4-3 (6.75 g, 92%) which was used for the next step without further purification. ¹H-NMR (300 MHz, CDCl₃) δ 4.53-4.49 (t, 1H), 3.59-3.42 (m, 2H), 3.42-3.39 (m, 2H), 2.43-2.41 (t, 2H), 1.96-1.92 (m, 2H), 1.58-1.54 (m, 5H), 1.32-1.27 (m, 23H), 0.90-0.85 (m, 6H).

### Synthesis of 6-bromohexyl 4,4-bis(octyloxy)butanoate (L4-5)

To an oven dried 100 mL round bottom flask containing a solution of 4,4-bis(octyloxy)butanoic acid L4-3 (1.5 g, 4.35 mmol) in dichloromethane (20 mL) under nitrogen was added EDC (1.25 g, 6.53 mmol), DMAP (0.106 g, 0.871 mmol) and DIPEA (2.25 g, 17.41 mmol). After completion of the addition, the mixture was stirred at room temperature for 10 min. 6-bromo-1-hexanol L4-4 (1.02 g, 5.66 mmol) was then added and the reaction mixture was stirred for 16 h at room temperature. The reaction mixture was diluted with dichloromethane and water and the resulting phases were separated. The aqueous phase was extracted again with EtOAc (50 mL). Combined organic extracts were washed with H₂O (60 mL×2) and dried over anhydrous MgSO₄. Filtration and concentration provided crude product which was purified by flash column chromatography (SiO₂: 0 to 10% ethyl acetate in hexanes gradient) to yield 6-bromohexyl 4,4-bis(octyloxy)butanoate L4-5 as colorless oil (0.74 g, 33%). ¹H-NMR (300 MHz, CDCl₃) δ 4.49 (t, 1H), 4.06 (t, 2H), 3.60-3.50 (m, 2H), 3.45-3.34 (m, 4H), 2.38 (t, 2H), 1.96-1.81 (m, 4H), 1.66-1.18 (m, 30H), 0.90-0.85 (m, 6H).

### Synthesis of ((4-hydroxybutyl)azanediyl)bis(hexane-6,1-diyl) bis(4,4-bis(octyloxy)butanoate) (Compound 14)

To a solution of compound L4-5 (0.74 g, 1.46 mmol) in CH₃CN/CPME (1:1, 10 mL) under nitrogen was treated with compound 4-amino-1-butanol (65.1 mg, 0.731 mmol) and followed by the addition of K₂CO₃ (0.708 g, 5.12 mmol) and KI (0.726 g, 4.38 mmol). The reaction mixture was heated at 80° C. for 18 h. After cooling to room temperature, the reaction mixture was filtered through Celite, washed with ethyl acetate, and the solvent was removed under vacuum to give the crude product which was purified by flash chromatography (SiO₂: DCM/MeOH 0-10%) to get final Compound 14 as colorless oil (0.3 g, 44%). ¹H-NMR (300 MHz, CDCl₃) δ 4.48 (t, 2H), 4.04 (t, 4H), 3.60-3.50 (m, 6H), 3.42-3.36 (m, 4H), 2.56-2.32 (m, 8H), 1.95-1.88 (q, 4H), 1.68-1.46 (m, 20H), 1.41-1.18 (m, 51H), 0.89-0.85 (t, 12H); CIMS m/z 942.7 [M+H]⁺; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.81 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=12.9 min, purity: 98.63%.

### Synthesis of Compound 49 (4-(Bis(9,9-bis(octyloxy)nonyl)amino)butan-1-ol)

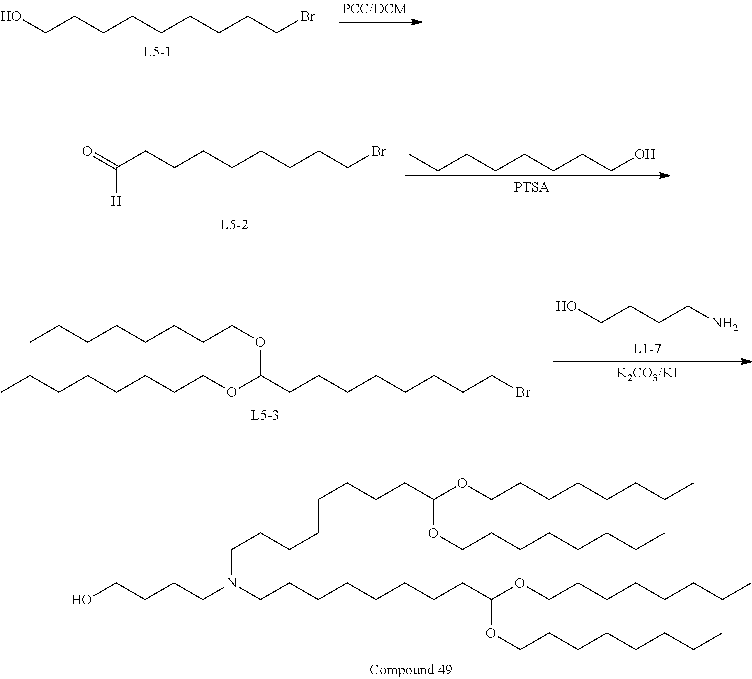

### Synthesis of 9-bromononanal (L5-2)

To an oven dried 1000 mL round bottom flask containing a solution of 9-bromononanol (5.0 g, 22.52 mmol) in dichloromethane (400 mL) was added Pyridinium chlorochromate (PCC) (7.74 g, 33.77 mmol) and the reaction mixture was stirred for 3 h at room temperature. The reaction mixture was then passed through a plug of Florisil. Water was added to the filtrate and the resulting phases were separated. The aqueous phase was extracted again with DCM (150 mL). Combined organic extracts were washed with aqueous sodium bicarbonate solution (150 mL) and H₂O (60 mL×2) and dried over anhydrous MgSO₄. Filtration and concentration provided crude L5-2 as colorless oil (4.95 g, 100%) which was used for the next step without further purification; ¹H-NMR (300 MHz, CDCl₃) δ 9.76 (s, 1H), 3.40 (m, 2H), 2.43 (m, 2H), 1.85-1.78 (m, 2H), 1.70-1.55 (m, 2H), 1.48-1.19 (m, 8H).

### Synthesis of 9-bromo-1,1-bis(octyloxy)nonane(L5-3)

To an oven dried 100 mL round bottom flask containing a solution of L5-2 (5.0 g, 22.61 mmol) in dichloromethane (150 mL) was added 1-octanol (8.83 g, 67.83 mmol), PTSA (0.584 g, 3.39 mmol) and sodium sulfate (9.63 g, 67.83 mmol). After completion of the addition, the mixture was stirred at room temperature for 18 h. Dichloromethane and water were then added to the reaction mixture and the resulting phases were separated. The aqueous phase was extracted again with DCM (150 mL). Combined organic extracts were washed with aqueous sodium bicarbonate solution (150 mL) and H₂O (60 mL×2) and dried over anhydrous MgSO₄. Filtration and concentration provided crude material which was purified by flash column chromatography (SiO₂: 0 to 20% EtOAc/Hexanes) to yield L5-3 (1.4 g, 14%); ¹H-NMR (300 MHz, CDCl₃) δ 4.45 (t, 1H), 3.57-3.50 (m, 2H), 3.43-3.34 (m, 4H), 1.87-1.80 (m, 2H), 1.62-1.51 (m, 6H), 1.41-1.20 (m, 30H), 0.92-0.85 (m, 6H).

### Synthesis of 4-(bis(9,9-bis(octyloxy)nonyl)amino)butan-1-ol (Compound 49)

To a solution of compound L5-3 (0.75 g, 1.62 mmol) in CH₃CN/CPME (1:1, 10 mL) under nitrogen was added 4-amino-1-butanol L1-7 (72 mg, 0.811 mmol) followed by the addition of K₂CO₃ (0.783 g, 5.67 mmol) and KI (0.807 g, 4.86 mmol). The reaction mixture was heated at 85° C. under nitrogen for 24 h. After cooling to room temperature, the reaction mixture was filtered through Celite, washed with ethyl acetate, and the solvent was removed under vacuum to give the crude product which was purified by flash chromatography (SiO₂: DCM/MeOH 0-10%) to provide Compound 49 (0.250 g, 36%) as slightly yellow oil. ¹H-NMR (300 MHz, CDCl₃) δ 4.44 (t, 2H), 3.56-3.53 (m, 6H), 3.41-3.35 (m, 4H), 2.44-2.39 (m, 6H), 1.64-1.20 (m, 81H), 0.89-0.85 (m, 12H). CIMS m/z 854.8 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20+2° C., detector: ELSD, t_{R}=11.99 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=15.45 min, purity: 91.24%.

### Synthesis of Compound 1 (undecyl 6-((7-((dioctylcarbamoyl)oxy) heptyl) (2-hydroxyethyl) amino) hexanoate)

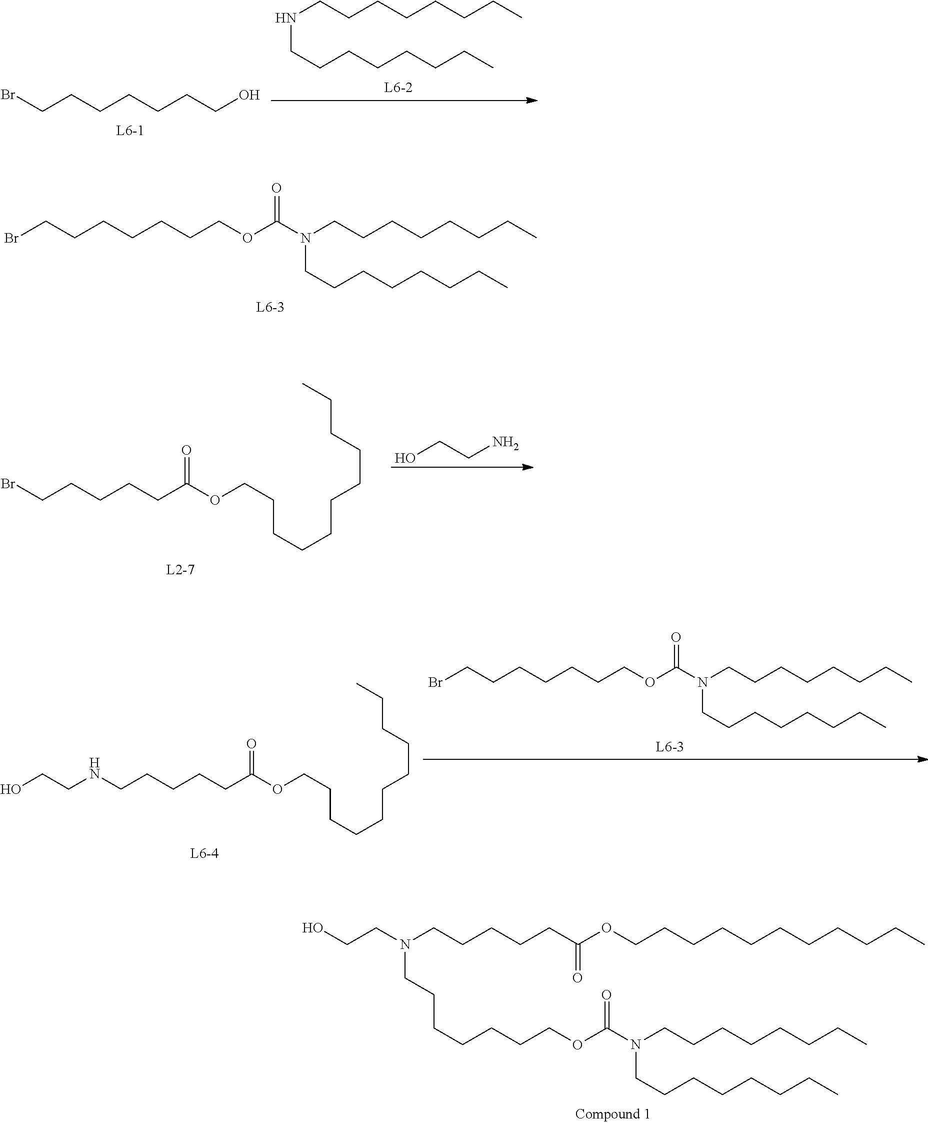

### Synthesis of 2,3,3a,4,7,7a-hexahydro-1H-isoindole (L6-3)

To a solution of compound L6-1 (1.0 g, 5.1 mmol) in DCM (20 mL), cooled in an ice-water bath under nitrogen, was added triphosgene (1.5 g, 11.2 mmol) over 5 min followed by the addition of DIPEA (1.98 g, 15.3 mmol) and stirred for 5 min. The reaction was allowed to warm to room temperature and stirred for 1 h and then compound L6-2 (1.2 g, 5.1 mmol) was added in one portion. The resulting mixture was allowed to warm to room temperature and stirred for 12 h. The solvent was removed in vacuo, and the residue was dissolved in ethyl acetate and washed with saturated NaHCO₃ and brine and dried (Na₂SO₄). The mixture was concentrated in vacuo to give crude product which was purified by silica gel column chromatography eluted with a gradient of hexane/ethyl acetate to obtain L6-3 as colorless oil (1.2 g, 50%). ¹H-NMR (300 MHz, CDCl₃) δ 4.32-4.25 (m, 2H), 4.02 (t, 2H), 3.41-3.36 (m, 3H), 3.15 (s, 3H), 1.86-1.70 (m, 5H), 1.60-1.25 (m, 27H), 0.86 (t, 6H); CIMS m/z 462.56 [M+H]⁺.

### Synthesis of undecyl 6-((2-hydroxyethyl) amino) hexanoate (L6-4)

A solution of compound L2-7 (6.0 g, 17.1 mmol) in ethanol (80 mL) was added dropwise to a solution of ethanolamine (31.4 g, 515 mmol) in EtOH (940 mL) at ambient temperature over 25 minutes. The reaction solution was heated at 60° C.-70° C. for 5 h. The reaction solution was allowed to cool to 35° C. by turning the heater off, and the reaction solution was concentrated in vacuo at 40° C. to give crude residue. The crude residue was diluted in TBME and then the TBME layer was separated from the ethanolamine layer. The ethanolamine layer was back extracted with TBME (200 mL). The combined TBME layers were washed with 5% NaHCO₃ solution and then concentrated in vacuo at 40° C. to give pale yellow oil. The crude product was purified by flash chromatography (SiO2: DCM/MeOH 0-100%, 1% NH₄OH) to give product L6-4 as colorless oil (2.3 g, 40%). ¹H-NMR (300 MHz, CDCl₃) δ 4.01 (t, 2H), 3.63 (t, 2H), 2.90 (s, 3H), 2.74 (t, 2H), 2.60 (t, 2H), 2.27 (t, 2H), 1.63-1.40 (m, 7H), 1.37-1.19 (m, 15H), 0.84 (t, 3H); CIMS m/z 330.3 [M+H]⁺.

### Synthesis of undecyl 6-((7-((dioctylcarbamoyl)oxy) heptyl) (2-hydroxyethyl) amino) hexanoate (Compound 1)

To a solution of compound L6-3 (500 mg, 1.08 mmol, 1 eq) in CPME (5 mL) and (ACN 5 mL), under nitrogen, was added compound L-2-8 (427 mg, 1.29 mmol, 1.2 eq), followed by the addition of K₂CO₃ (597 mg, 4.3 mmol, 4 eq) and KI (179 mg, 1.08 mmol, 1 eq). The reaction mixture was heated at 60° C. for 18 h. After cooling to room temperature, the reaction mixture was filtered through Celite, washed with ethyl acetate, and the solvent was removed under vacuum to give the crude product which was purified by flash chromatography (80 g SiO₂: 0 to 100% ethyl acetate in hexane with 0 to 30% triethylamine gradient) to obtain Compound 1 as colorless oil (0.15 g, 20%). ¹H-NMR (300 MHz, CDCl₃) δ 4.06-4.0 (m, 4H), 3.50-3.49 (m, 3H), 3.17 (s, 3H), 2.55 (t, 2H), 2.45-2.39 (m, 5H), 2.28 (t, 3H), 1.64-1.55 (m, 7H), 1.49-1.39 (m, 7H), 1.34-1.24 (m, 43H), 0.84 (t, 9H)); CIMS m/z 711.3 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.2 min, purity: 92.0%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=12.6 min, purity: 91.05%.

### Synthesis of Compound 3 ((4-hydroxybutyl) azanediyl) bis(hexane-6,1-diyl) bis(dioctylcarbamate)

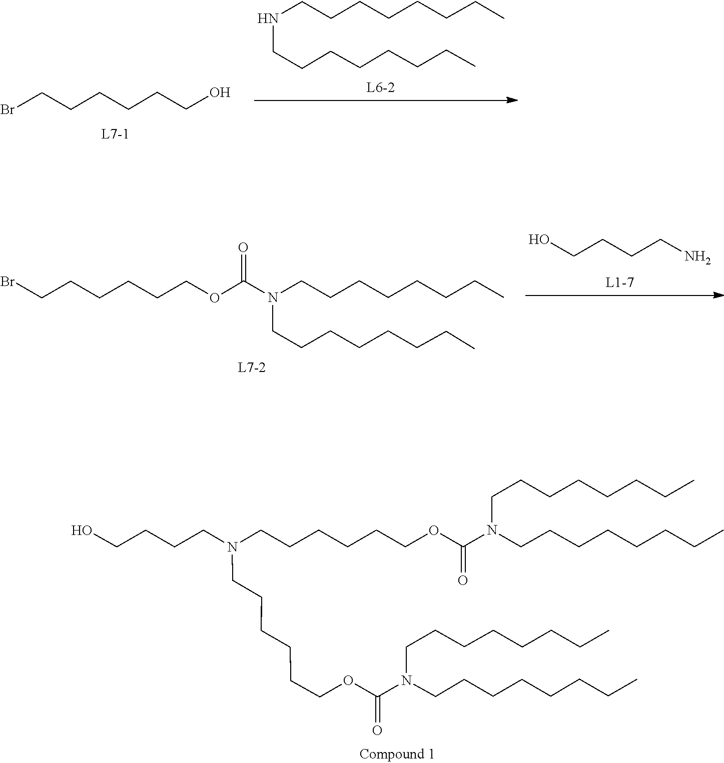 [SMILES: CCCCCCCCNCCCCCCCC ; OCCCCCCBr ; CCCCCCCCN(CCCCCCCC)C(=O)OCCCCCCBr ; NCCCCO ; CCCCCCCCN(CCCCCCCC)C(=O)OCCCCCCN(CCCCO)CCCCCCOC(=O)N(CCCCCCCC)CCCCCCCC]

### Synthesis of 6-bromohexyl dioctylcarbamate (L7-2)

To a solution of compound L7-1 (1.0 g, 5.52 mmol) in DCM (20 mL), cooled in an ice-water bath and under nitrogen, was treated with triphosgene (1.63 g, 5.52 mmol) over 5 min followed by the addition of DIPEA (2.8 mL, 16.5 mmol) and stirred for 5 min, the mixture was allowed to warm to temperature and stirred for 1 h and then compound-L6-2 was added in one portion. The resulting mixture was allowed to warm to room temperature and stir for 12 h. The solvent was removed in vacuo, and the residue was dissolved in ethyl acetate and washed with saturated NaHCO₃ and brine and dried (Na₂SO₄). Concentration in vacuo gave crude product which was purified by silica gel column chromatography eluted with a gradient of hexane/ethyl acetate to obtain final Compound L7-2 (1.48 g, 60%); ¹H-NMR (300 MHz, CDCl₃) δ 4.04 (t, 2H), 3.39 (t, 2H), 3.16 (s, 4H), 1.90-1.74 (m, 2H), 1.64-1.58 (m, 2H), 1.51-1.37 (m, 7H), 1.36-1.12 (m, 21H), 0.87 (t, 6H). CIMS m/z 449.2 [M+H]⁺.

### Synthesis of ((4-hydroxybutyl) azanediyl) bis(hexane-6,1-diyl) bis(dioctylcarbamate) (Compound 3)

To a solution of compound L1-7 (0.05 g, 0.5 mmol) in CPME (5 mL) and (ACN 5 mL), under nitrogen, was added compound L7-2 (0.503 g, 1.1 mmol) and followed by the addition of K₂CO₃ (0.310 g, 2.2 mmol, 4 eq) and KI (0.093 g, 0.56 mmol). The reaction mixture was heated at 80° C. for 18 h. After cooling to room temperature, the reaction mixture was filtered through Celite, washed with ethyl acetate, and the solvent was removed under vacuum to give the crude product which was purified by flash chromatography (SiO₂: DCM/MeOH 0-10%) to give Compound 3 (0.2 g, 45%) as colorless oil; ¹H-NMR (300 MHz, CDCl₃) δ 4.03 (t, 4H), 3.61 (t, 2H), 3.15-3.14 (m, 7H), 2.71-2.69 (m, 5H), 1.78-1.75 (m, 10H), 1.71-1.58 (m, 15H), 1.41-1.26 (m, 46H), 0.84 (t, 12H); CIMS m/z 824.7 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20+2° C., detector: ELSD, t_{R}=11.4 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=13.8 min, purity: 99.38%.

### Synthesis of Compound 20 (undecyl 6-((8-(2,2-dioctylhydrazineyl)-8-oxooctyl)(2-hydroxyethyl)amino)hexanoate)

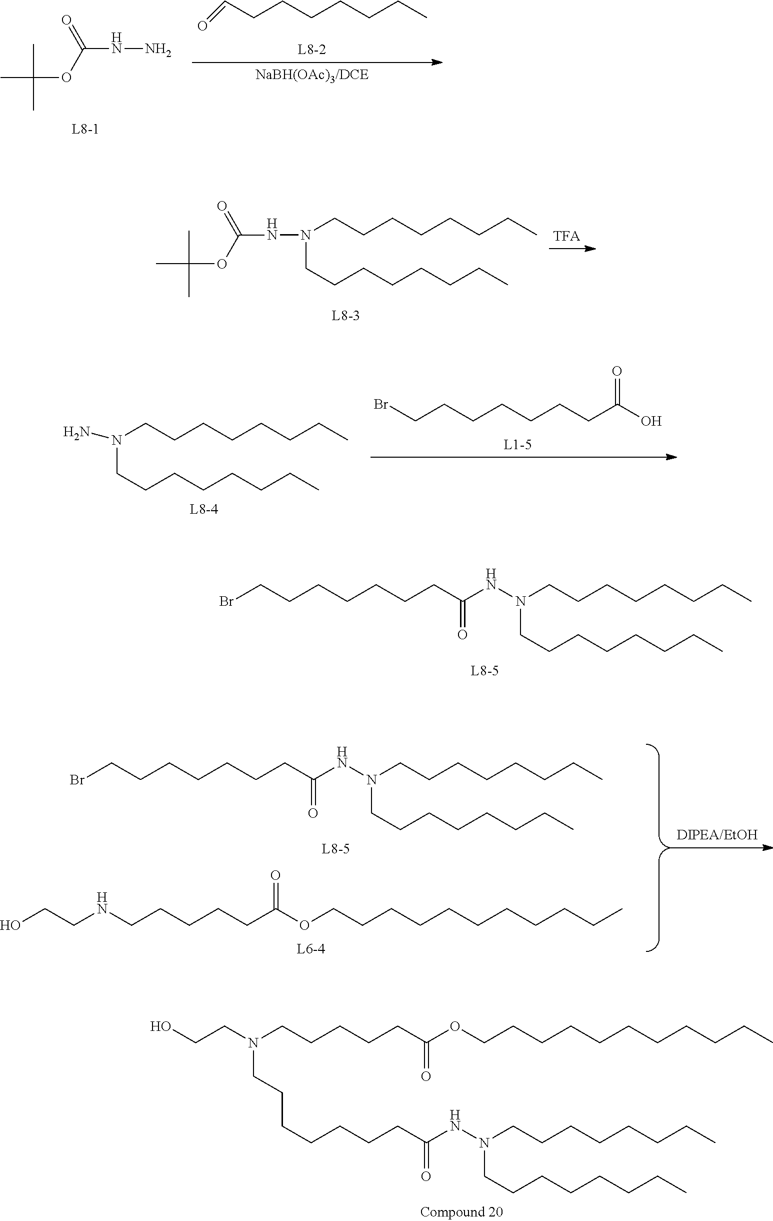

### Synthesis of tert-butyl 2,2-dioctylhydrazine-1-carboxylate (L8-3)

To an oven dried 500 mL round bottom flask, to a solution of tert-butyl carbazate (3.0 g, 22.70 mmol), 1-octanal (3.64 g, 28.37 mmol) in dichloroethane (100 mL), was added NaBH(OAc)₃ (24.05 g, 113.5 mmol) and the reaction mixture was stirred for 24 h at room temperature. The reaction mixture was then diluted with DCM (300 mL). Water was added and the resulting two phases were separated. The aqueous phase was extracted again with DCM (150 mL). Combined organic extracts were washed with bicarbonate solution (150 mL), and H₂O (60 mL×2) and dried over anhydrous MgSO₄. Filtration and concentration provided crude material which was purified by flash column chromatography (SiO₂: 0 to 35% EtOAc/Hexanes) to yield L8-3 (3.6 g, 44%); ¹H-NMR (300 MHz, CDCl₃) Rotamers Observed δ 5.16 (bs, 0.68H), 4.71 (bs, 0.23H), 2.70-2.52 (m, 4H), 1.55-1.38 (m, 13H), 1.38-1.15 (m, 20H), 0.92-0.84 (t, 6H). CIMS m/z 357.3 [M+H]⁺.

### Synthesis of 1,1-dioctylhydrazine (L8-4)

To a solution of L8-3 (3.6 g, 10.08 mmol) in dichloromethane (40 mL) was added TFA (20 mL) at 0° C. and the reaction mixture was stirred at room temperature for 16 h. The volatile components were removed under reduced pressure and the crude L8-4 (2.7 g) was used for the next step without further purification. ¹H-NMR (300 MHz, CDCl₃) δ 6.50 (b, 1H), 3.30 (b, 1H), 2.74 (t, 2H), 2.51-2.38 (m, 2H), 1.58-1.46 (m, 3H), 1.32-1.12 (m, 20H), 0.89-0.84 (m, 6H). CIMS m/z 257.2 [M+H]⁺.

### Synthesis of 8-bromo-N′,N′-dioctyloctanehydrazide (L8-5)

To an oven dried 100 mL round bottom flask containing a solution of L8-4 (0.400 g, 1.561 mmol) and bromo-octanoic acid (0.485 g, 2.185 mmol) in dichloromethane (20 mL) was added EDC (0.449 g, 2.341 mmol) and DMAP (0.038 g, 0.312 mmol). After completion of the addition, the mixture was stirred at room temperature for 16 h. The reaction mixture was diluted with water and the resulting phases were separated. The aqueous phase was extracted with DCM (50 mL). Combined organic extracts were washed with H₂O (60 mL×2) and dried over anhydrous MgSO₄. Filtration and concentration provided crude product which was purified by flash column chromatography (SiO₂: 0 to 50% ethyl acetate in hexanes gradient) to yield L8-5 as a white solid (0.24 g, 33%). ¹H-NMR (300 MHz, CDCl₃) δ 5.78 (s, 0.19H), 5.64 (s, 0.61H), 3.39 (t, 2H), 2.70-2.64 (m, 2H), 2.43-2.37 (m, 2H), 2.10 (t, 0.65H), 1.87-1.80 (m, 2H), 1.63-1.56 (m, 2H), 1.49-1.20 (m, 30H), 1.03 (t, 1H), 0.88-0.85 (m, 6H). CIMS m/z 461.2, 463.2 [M+H]⁺.

### Synthesis of undecyl 6-((8-(2,2-dioctylhydrazineyl)-8-oxooctyl)(2-hydroxyethyl)amino)hexanoate (Compound 20)

To a solution of compound L8-5 (0.240 g, 0.52 mmol) in EtOH (5 mL) under nitrogen was added compound L6-4 (223 mg, 0.676 mmol) and followed by the addition of DIPEA (0.235 g, 1.82 mmol). The reaction mixture was heated at 80° C. for 24 h. After cooling to room temperature, the reaction mixture was evaporated under vacuum to give the crude product which was purified by flash chromatography (SiO₂: DCM/MeOH 0-10%) to give Compound 20 (0.078 g, 30%); ¹H-NMR (300 MHz, CDCl₃) Rotamers observed δ 6.02 (s, 0.23H), 5.71 (s, 0.5H), 4.05 (t, 2H), 3.98-3.86 (m, 2H), 3.15-2.90 (m, 5H), 2.73-2.64 (m, 2H), 2.45-2.29 (m, 4H), 2.11 (t, 0.46H), 1.89-1.56 (m, 10H), 1.46-1.15 (m, 46H), 0.90-0.85 (m, 9H). CIMS m/z 710.6 [M+H]⁺. Analytical HPLC column: Agilent Zorbax SB-C18, 5 m, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.4 min, purity: 99.90%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=9.6 min, purity: 93.12%.

### Synthesis of Compound 18 (7,7′-((4-hydroxybutyl)azanediyl)bis(N′,N′-dioctylheptanehydrazide)

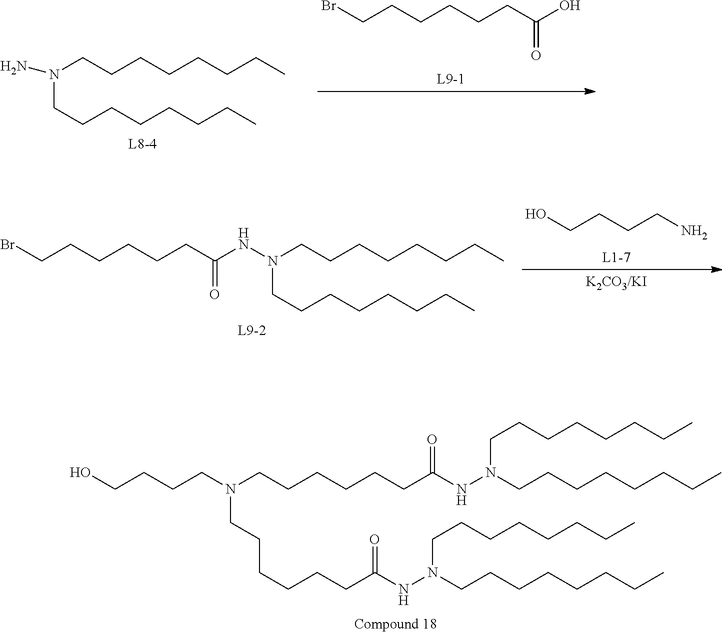 [SMILES: O=C(O)CCCCCCBr ; CCCCCCCCN(N)CCCCCCCC ; NCCCCO ; CCCCCCCCN(CCCCCCCC)NC(=O)CCCCCCBr ; CCCCCCCCN(CCCCCCCC)NC(=O)CCCCCCN(CCCCO)CCCCCCC(=O)NN(CCCCCCCC)CCCCCCCC]

### Synthesis of 7-bromo-N,N-dioctylheptanehydrazide (L9-2)

To an oven dried 100 mL round bottom flask containing a solution of L8-4 (1.25 g, 4.88 mmol) and bromo-heptanoic acid (1.53 g, 7.32 mmol) in dichloromethane (20 mL) was added DCC (2.01 g, 9.76 mmol) and DMAP (0.06 g, 0.488 mmol). After completion of the addition, the mixture was stirred at room temperature for 16 h. The reaction mixture was then diluted with water and the organic phase was separated. The aqueous phase was extracted with DCM (50 mL). Combined organic extracts were washed with H₂O (60 mL×2) and dried over anhydrous MgSO₄. Filtration and concentration provided crude material which was purified by flash column chromatography (SiO₂: 0 to 30% ethyl acetate in hexanes gradient) to yield L9-2 as colorless oil (0.59 g, 27%); ¹H-NMR (300 MHz, CDCl₃) Rotamers Observed δ 5.78 (s, 0.3H), 5.66 (s, 1.2H), 3.52 (t, 4H), 3.40 (q, 2H), 2.74-2.60 (m, 5H), 2.48-2.35 (m, 7H), 2.14-2.06 (t, 1H), 1.82-1.78 (m, 3H), 1.68-1.54 (m, 7H), 1.52-1.36 (m, 12H), 1.32-1.15 (m, 55H), 0.88-0.85 (m, 16H). CIMS m/z 447.2, 449.2 [M+H]⁺.

### Synthesis of 7,7′-((4-hydroxybutyl)azanediyl)bis(N,N-dioctylheptanehydrazide) (Compound 18)

To a solution of compound L9-2 (0.500 g, 1.12 mmol) in CPME (5 mL) and (ACN 5 mL), under nitrogen, was treated with compound 4-amino-1-butanol (40 mg, 0.811 mmol) and followed by the addition of K₂CO₃ (0.464 g, 3.36 mmol) and KI (0.557 g, 3.36 mmol). The reaction mixture was heated at 90° C. for 18 h. After cooling to room temperature, the reaction mixture was filtered through Celite, washed with ethyl acetate, and the solvent was removed under vacuum to give the crude product which was purified by flash chromatography (SiO₂: DCM/MeOH 0-10%) to provide Compound 18 (0.054 g, 12%) as yellow oil; ¹H-NMR (300 MHz, CDCl₃) δ 6.40 (s, 0.3H), 6.25 (s, 0.15H), 5.70 (s, 1.13H), 3.68-3.58 (m, 2H), 2.80-2.60 (m, 9H), 2.48-2.36 (m, 5H), 2.12 (t, 1H), 1.80-1.15 (m, 73H), 0.90-0.84 (m, 12H). CIMS m/z 822.7 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=5.26 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20+2° C., detector: CAD, t_{R}=8.86 min, purity: 96.15%.

### Synthesis of Compound 6 ((4-((2-(methylamino)-3,4-dioxocyclobut-1-en-1-yl)amino)butyl)azanediyl)bis(hexane-6,1-diyl) bis(2-hexyldecanoate)

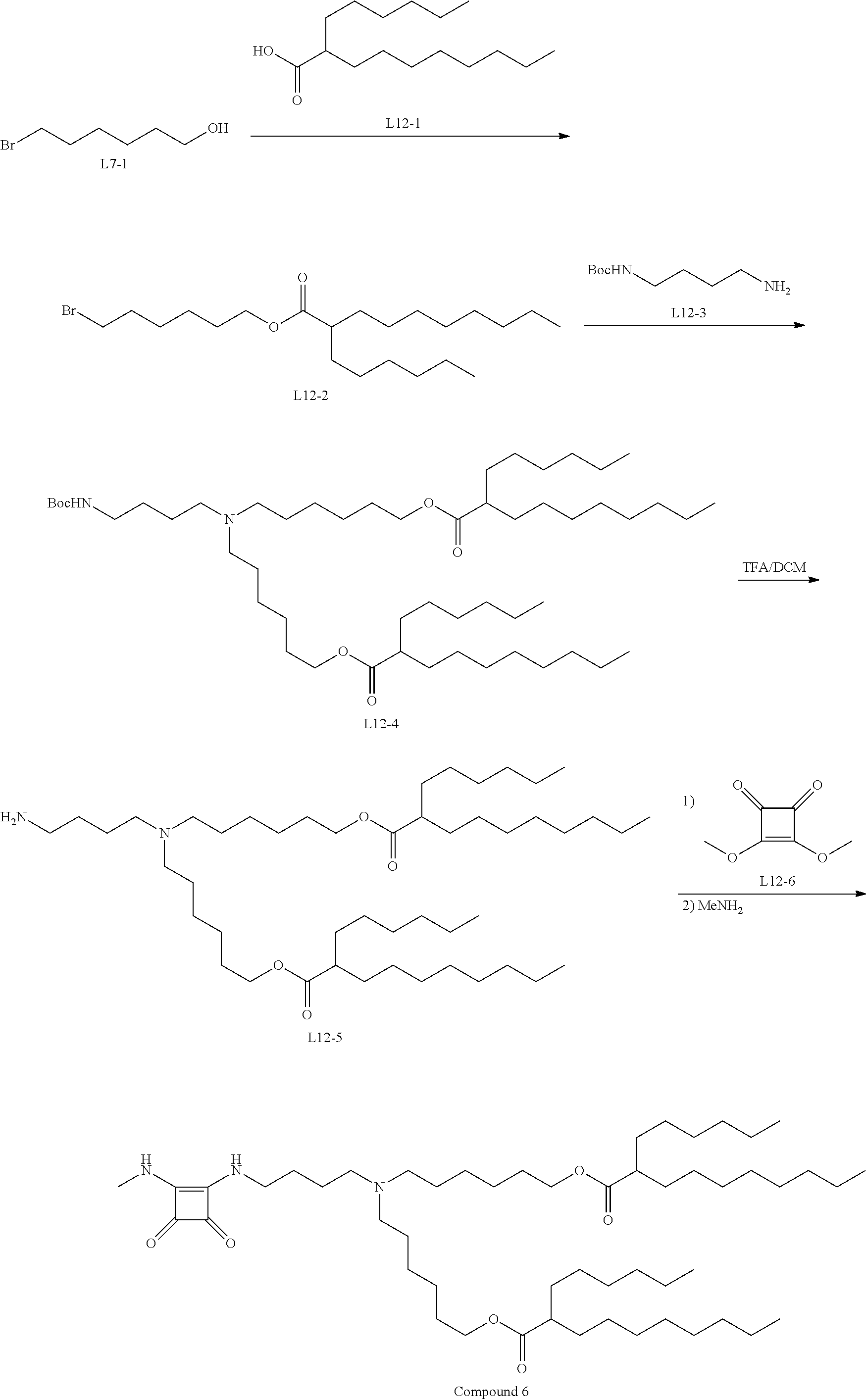

### Synthesis of 6-bromohexyl 2-hexyldecanoate (L12-2)

To an oven dried 100 mL round bottom flask containing a solution of 2-hexyldecanoic acid L12-1 (5.0 g, 19.50 mmol) in dichloromethane (50 mL) was added EDC (5.61 g, 29.25 mmol) and DMAP (0.476 g, 3.90 mmol). After completion of the addition, the mixture was stirred at room temperature for 10 min. 6-bromo-1-hexanol L7-1 (3.5 g, 19.50 mmol) was then added and the reaction mixture was stirred at room temperature for 16 h. The reaction was diluted with water and the resulting phases were separated. The aqueous phase was extracted with EtOAc (50 mL×2). Combined organic extracts were washed with H₂O (60 mL×2) and dried over anhydrous MgSO₄. Filtration and concentration provided crude material which was purified by flash column chromatography (SiO₂: 0 to 30% ethyl acetate in hexanes gradient) to yield 6-bromohexyl 2-hexyldecanoate L12-2 as colorless oil (3.8 g, 47%); CIMS m/z 419.2, 421.2 [M+H]⁺.

### Synthesis of ((4-((tert-butoxycarbonyl)amino)butyl)azanediyl)bis(hexane-6,1-diyl) bis(2-hexyldecanoate) (L12-4)

To a solution of compound L12-2 (2.0 g, 4.77 mmol) in CPME (5 mL) and (ACN 5 mL), under nitrogen, was treated with compound tert-butyl (4-aminobutyl)-carbamate (404 mg, 2.15 mmol) and followed by the addition of K₂CO₃ (2.31 g, 16.69 mmol) and KI (2.37 g, 14.31 mmol). The reaction mixture was heated at 85° C. for 18 h. After cooling to room temperature, the reaction mixture was filtered through Celite, washed with ethyl acetate, and the solvent was removed under vacuum to give the crude (2.6 g, 100%) product which was used for the next step without further purification; ¹H-NMR (300 MHz, CDCl₃) δ 5.05 (m, 1H), 4.05 (t, 4H), 3.19-3.03 (m, 2H), 2.48-2.25 (m, 6H), 1.66-1.17 (m, 79H), 0.89-0.84 (m, 12H); CIMS m/z 865.7 [M+H]⁺.

### Synthesis of ((4-aminobutyl)azanediyl)bis(hexane-6,1-diyl) bis(2-hexyldecanoate) (L12-5)

To a solution of L12-4 (2.06 g, 2.38 mmol) in dichloromethane (20 mL) was added TFA (5 mL) at 0° C. and allowed the reaction to room temperature and stirred for 4 h. The Solvent was removed on rotavapor, crude purified by flash column chromatography (SiO₂: 0 to 10% MeOH/DCM with 1% NH₄OH) to yield L12-5 as sticky oil (1.5 g, 82%); ¹H-NMR (300 MHz, CDCl₃) δ 4.05 (t, 4H), 2.75-2.71 (m, 2H), 2.45-2.27 (m, 8H), 1.66-1.15 (m, 74H), 0.89-0.84 (m, 12H). CIMS m/z 765.7 [M+H]⁺.

### Synthesis of ((4-((2-(methylamino)-3,4-dioxocyclobut-1-en-1-yl)amino)butyl)azanediyl)bis(hexane-6,1-diyl) bis(2-hexyldecanoate) (Compound 6)

To a solution of L12-5 (500 mg, 0.653 mmol) in diethyl ether (50 mL) was added 3,4-dimethoxy cyclobutene-1,2-dione (139 mg, 0.980 mmol) at 0° C. and allowed the reaction to room temperature and stirred for 3 h. TLC showed all starting material was consumed. To the reaction mixture was added methyl amine (2.0 M) in methanol (300 mg, 6.53 mmol) and continued stirring the reaction overnight at room temperature. Solvent was removed on rotavapor, and the crude was purified by flash column chromatography (SiO₂: 0 to 10% MeOH/DCM with 1% NH₄OH) to yield Compound 6 as sticky gum (300 mg, 53%); ¹H-NMR (300 MHz, CDCl₃) δ 8.20 (b, 1H), 7.45 (b, 1H), 4.06 (t, 4H), 3.72-3.68 (m, 2H), 3.31 (d, 3H), 3.20-2.94 (m, 6H), 2.33-2.27 (m, 2H), 1.95-1.80 (m, 2H), 1.73-1.56 (m, 18H), 1.53-1.41 (m, 12H), 1.40-1.24 (m, 42H), 0.89-0.84 (t, 12H). CIMS m/z 874.7 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.84 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=14.05 min, purity: 96.59%.

### Synthesis of Compound 37 (bis(3-pentyloctyl) 9-((2-((3-(dimethylamino)propyl)amino)-3,4-dioxocyclobut-1-en-1-yl)amino)heptadecanedioate)

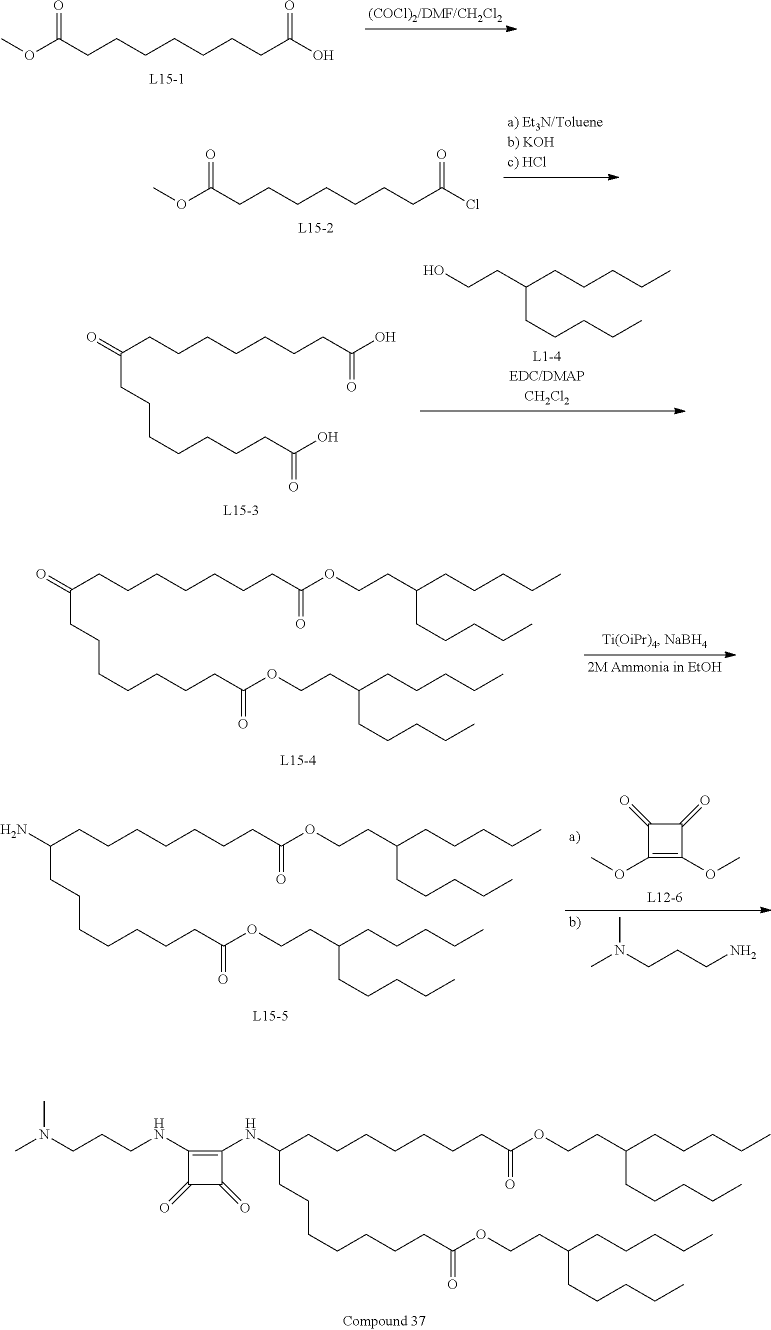

### Synthesis of methyl 9-chloro-9-oxononanoate (L15-2)

To a solution of L15-1 (12 g, 59 mmol) in anhydrous DCM (40 mL) was added anhydrous DMF (1 mL). With ice bath cooling, a solution of oxalyl chloride (8.2 g, 65 mmol) in anhydrous DCM (10 mL) was dropped in under nitrogen with stirring. The resulting mixture was then stirred at room temperature under nitrogen overnight. The reaction mixture was concentrated and co-evaporated with anhydrous toluene to give L15-2 as colorless oil (11.6 g, 97%). ¹H-NMR (300 MHz, CDCl₃) δ 3.63 (s, 3H), 2.85 (t, J=7.2 Hz, 2H), 2.27 (t, J=7.5 Hz, 2H), 1.77-1.48 (m, 4H), 1.41-1.19 (m, 6H).

### Synthesis of 9-oxoheptadecanedioic acid (L15-3)

To an ice bath cooled solution of L15-2 (11 g, 50 mmol) in anhydrous toluene (85 mL) was added triethylamine (5 g, 50 mmol) in 10 min with stirring while keeping the reaction temperature below 25° C. After addition finished, the reaction mixture temperature was brought to 35-40° C. during 15-20 min with a warm water bath. After the temperature reached to 40° C., the water bath was removed, and the reaction mixture was stirred for 1 h. It was then filtered through a short pad of Celite and the Celite was rinsed with toluene (25 mL). The combined filtrates were evaporated to give an oil residue which was mixed with 2N aq. KOH (42 mL). The mixture was refluxed for 6 h and then cooled in ice-bath. The aqueous layer was washed with ether (35 mL×3) and acidified with concentrated HCl to pH 4. After cooling the mixture in ice bath for 1 h, the precipitates were filtered, washed with ice cold water and dried to yield L15-3 as an off-white solid (6.0 g, 71%). ¹H-NMR (300 MHz, DMSO-d6) δ 2.38 (t, J=7.1 Hz, 4H), 2.18 (t, J=7.4 Hz, 4H), 1.53-1.35 (m, 8H), 1.33-1.02 (m, 12H).

### Synthesis of bis(3-pentyloctyl) 9-oxoheptadecanedioate (L15-4)

To a solution of L15-3 (1.8 g, 5.99 mmol) and L1-4 (2.52 g, 12.58 mmol) in DCM (20 mL) at room temperature was added DMAP (70 mg) and the reaction was stirred for 10 min followed by addition of EDC (2.45 g, 12.76 mmol). The reaction was stirred for 48 h at RT under nitrogen atmosphere. The reaction mixture was diluted with DCM (100 mL) and washed with saturated NaHCO₃ solution (2×25 mL), water (25 mL), and brine (25 mL). The organic layer was dried over anhydrous Na₂SO₄. Filtration and concentration yielded crude product which was purified by flash column chromatography (SiO₂: 5 to 8% ethyl acetate in hexane gradient) to yield L15-4 as colorless oil (2.1 g, 54%). ¹H-NMR (300 MHz, CDCl₃) δ 4.09-4.04 (t, 4H), 2.39-2.34 (t, 4H), 2.29-2.24 (t, 4H), 1.59-1.52 (m, 12H), 1.29-1.25 (m, 46H), 0.89-0.85 (t, 12H). CIMS m/z 680.6 [M+H]⁺.

### Synthesis of bis(3-pentyloctyl) 9-aminoheptadecanedioate (L15-5)

To a solution of L15-4 (0.5 g, 0.736 mmol) in 2M ammonia in EtOH (2.0 mL) solution was added titanium (IV) isopropoxide (0.46 mL, 1.509 mmol) dropwise at ambient temperature. The reaction was stirred for 6 h, progress of imine formation was monitored by TLC/mass spec. After completion of the imine formation, sodium borohydride (56 mg, 1.472 mmol) was added to the reaction portion wise. Addition of borohydride was exothermic, therefore the reaction mixture was cooled over the course of the reaction. The reaction was further stirred overnight at RT. The reaction mixture was cooled in an ice bath and quenched by adding NH₄OH solution (white precipitate formed). The reaction mixture was filtered through celite, and washed with EtOAc. Solvent was removed on in vacuo, and the crude material was purified by flash column chromatography (SiO₂: 5 to 7% MeOH/DCM (1% NH₄OH) to yield L15-5 as colorless oil (300 mg, 60%). ¹H-NMR (300 MHz, CDCl₃) δ 4.07 (t, 4H), 2.68 (s, 1H), 2.27 (t, 4H), 1.60-1.55 (m, 8H), 1.53-1.25 (m, 54H), 0.90-0.85 (t, 12H). CIMS m/z 681.6 [M+H]⁺.

### Synthesis of bis(3-pentyloctyl) 9-((2-((3-(dimethylamino)propyl)amino)-3,4-dioxocyclobut-1-en-I-yl)amino)heptadecanedioate (Compound 37)

To a solution of L15-5 (200 mg, 0.294 mmol) in diethyl ether (20 mL) was added 3,4-dimethoxy cyclobutene-1,2-dione (63 mg, 0.441 mmol) at 0° C. and the reaction was allowed to warm room temperature and stirred for 3 h. Additional 3,4-dimethoxy cyclobutene-1,2-dione (63 mg, 0.441 mmol) was added to the reaction mixture and stirred for 2 h. To the reaction mixture was added N,N-dimethyl-1,3-propanediamine (300 mg, 2.94 mmol) and stirring was continued overnight at room temperature. Solvent was removed in vacuo, and crude material was purified by flash column chromatography (SiO₂: 6 to 8% MeOH/DCM (1% NH₄OH) to yield Compound 37 as sticky gum (132 mg, 52%). ^{J}H-NMR (300 MHz, CDCl₃) δ 4.07 (t, 4H), 3.70 (s, 1H), 2.60 (s, 2H), 2.25-2.38 (s, 8H), 2.27 (t, 4H), 1.88-1.72 (m, 6H), 1.55 (m, 8H), 1.28-1.24 (m, 52H), 0.90-0.85 (t, 12H). CIMS m/z 860.7 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.85 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=14.63 min, purity: 95.25%.

### Synthesis of Compound 50 ((3-hydroxypropyl)azanediyl)bis(hexane-6,1-diyl) bis(4,4-bis(octyloxy)butanoate)

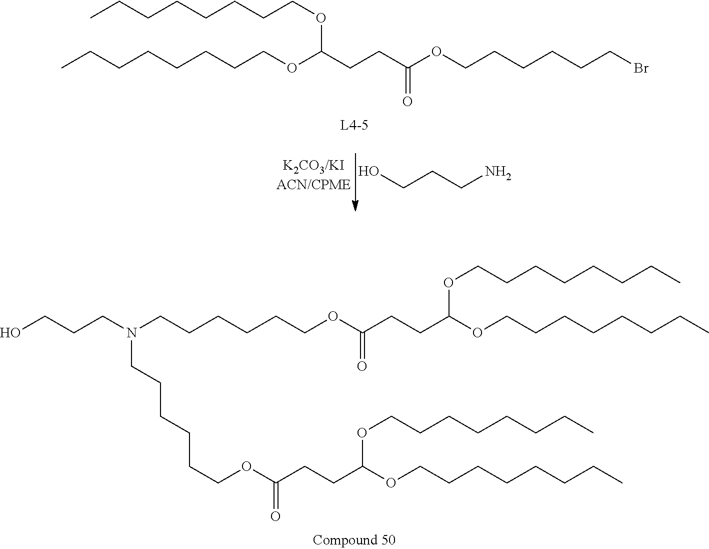 [SMILES: CCCCCCCCOC(CCC(=O)OCCCCCCBr)OCCCCCCCC ; NCCCO ; CCCCCCCCOC(CCC(=O)OCCCCCCN(CCCO)CCCCCCOC(=O)CCC(OCCCCCCCC)OCCCCCCCC)OCCCCCCCC]

### Synthesis of ((3-hydroxypropyl)azanediyl)bis(hexane-6,1-diyl) bis(4,4-bis(octyloxy)butanoate) (Compound 50)

To a solution of L4-5 (1.5 g, 2.96 mmol) and 3-amino-1-propanol (111 mg, 1.48 mmol) in CH₃CN (5 mL) and CPME (5 mL) at room temperature under nitrogen was added K₂CO₃ (1.23 g, 8.87 mmol) and KI (1.47 g, 8.87 mmol). The reaction mixture was then heated at 85° C. for 18 h. After cooling to room temperature, the reaction mixture was filtered through Celite, and washed with ethyl acetate, and the solvent was removed under vacuum to give the crude product which was purified by flash chromatography (SiO2:MeOH in DCM 0-10% gradient) to yield Compound 50 as colorless oil (0.685 g, 50%). ¹H-NMR (300 MHz, CDCl₃) δ; 4.49 (t, 2H), 4.05 (t, 4H), 3.78 (t, 2H), 3.60-3.52 (m, 4H), 3.43-3.36 (m, 4H), 2.67-2.60 (m, 2H), 2.42-2.32 (m, 8H), 1.92 (q, 4H), 1.67-1.39 (m, 20H), 1.38-1.10 (m, 47H), 0.87 (t, 12H); CIMS m/z [M+H]⁺ 928.9; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.60 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=14.23 min, purity: 97.97%.

### Synthesis of Compound 54 ((3-(((benzyloxy)carbonyl)amino)propyl)azanediyl)bis(hexane-6,1-diyl) bis(4,4-bis(octyloxy)butanoate) (Compound 62) and ((3-(3,3-dimethylthioureido)propyl)-azanediyl)bis(hexane-6,1-diyl) bis(4,4-bis(octyloxy)butanoate)

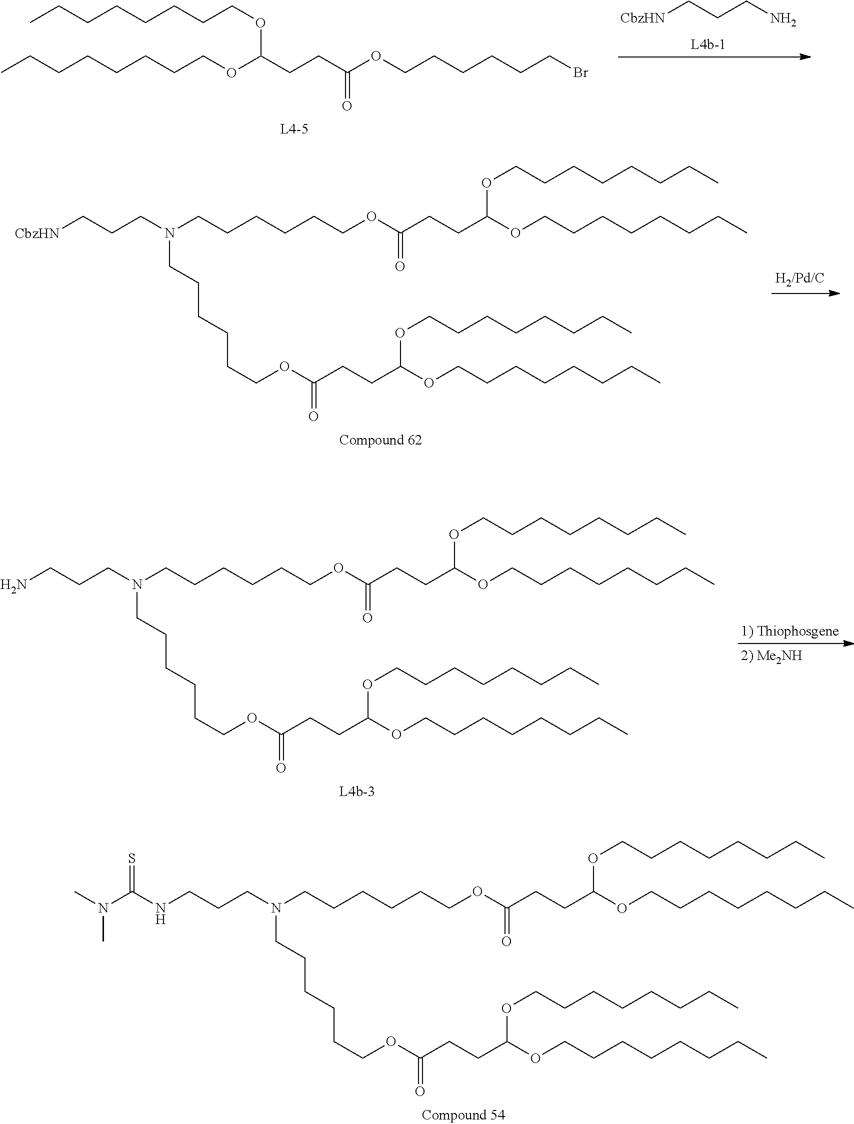

### Synthesis of ((3-(((benzyloxy)carbonyl)amino)propyl)azanediyl)bis(hexane-6,1-diyl) bis(4,4-bis(octyloxy)butanoate) (Compound 62)

To a solution of compound L4-5 (4.5 g, 8.87 mmol) in CH₃CN (10 mL) and CPME (10 mL) under nitrogen was added benzyl (3-aminopropyl)carbamate (994 mg, 4.43 mmol), followed by the addition of K₂CO₃ (3.68 g, 26.60 mmol) and KI (4.41 g, 26.60 mmol). The reaction mixture was heated at 85° C. for 18 h. After cooling to room temperature, the reaction mixture was filtered through Celite, washed with ethyl acetate, and the solvent was removed under vacuum to give the crude product which was purified by flash chromatography (SiO₂:MeOH in DCM 0-5% gradient) to yield compound Compound 62 as colorless oil (1.2 g, 25%). ¹H-NMR (300 MHz, CDCl₃) δ; 7.33-7.28 (m, 5H), 5.08 (s, 2H), 4.47 (t, 2H), 4.02 (t, 4H), 3.58-3.52 (m, 4H), 3.42-3.29 (m, 4H), 3.29-3.25 (m, 2H), 2.36 (t, 4H), 1.93-1.88 (m, 4H), 1.62-1.45 (m, 16H), 1.90-1.40 (m, 47H), 0.87 (t, 12H); CIMS m/z [M+H]⁺ 1061.9; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.81 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.62 min, purity: 96.66%.

### Synthesis of ((3-aminopropyl)azanediyl)bis(hexane-6,1-diyl) bis(4,4-bis(octyloxy)butanoate) (L4b-3)

A mixture of Compound 62 (0.85 g, 0.80 mmol), Pd/C (0.25 g) and acetic acid (0.144 g, 2.40 mmol) in ethyl acetate (15 mL) was stirred for 4 h under H2 balloon. The reaction was filtered through a celite pad, and the solvent was removed in vacuo. Crude material was dissolved in DCM (25 mL) and washed with bicarbonate solution. The organic phase was separated, dried and evaporated to give L4b-3 as a sticky oil (330 mg, 45%) which was used for the next step without further purification.

### Synthesis of ((3-(3,3-dimethylthioureido)propyl)azanediyl)bis(hexane-6,1-diyl) bis(4,4-bis(octyloxy)butanoate) (Compound 54)

To a solution of L4b-3 (400 mg, 0.431 mmol) in diethyl ether (30 mL) at 0° C. was added triethylamine (218 mg, 2.156 mmol) and thiophosgene (146 mg, 1.293 mmol). The reaction mixture was warmed to room temperature and stirred for 3 h. The volatile components were removed under reduced pressure and to the residue was added methyl amine (2.0 M) in THF (2.16 mL, 4.31 mmol). Stirring continued at room temperature for 16h. Solvent was removed in vacuo, and crude material was purified by flash column chromatography (SiO₂: 0 to 10% MeOH/DCM with 1% NH₄OH) to yield Compound 54 as sticky gum (152 mg, 35%). ¹H-NMR (300 MHz, CDCl₃) δ. 4.49 (t, 2H), 4.05 (t, 4H), 3.80 (b, 2H), 3.62-3.52 (m, 4H), 3.43-3.18 (m, 8H), 3.00 (b, 2H), 2.38 (t, 4H), 1.96-1.89 (m, 4H), 1.68-1.10 (m, 73H), 0.87 (t, 12H); CIMS m/z [M+H]⁺ 1014.8. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=7.96 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.77 min, purity: 98.33%.

### Synthesis of Compound 56 ((3-((2-(methylamino)-3,4-dioxocyclobut-1-en-1-yl)amino)propyl)azanediyl) bis(hexane-6,1-diyl) bis(4,4-bis(octyloxy)butanoate)

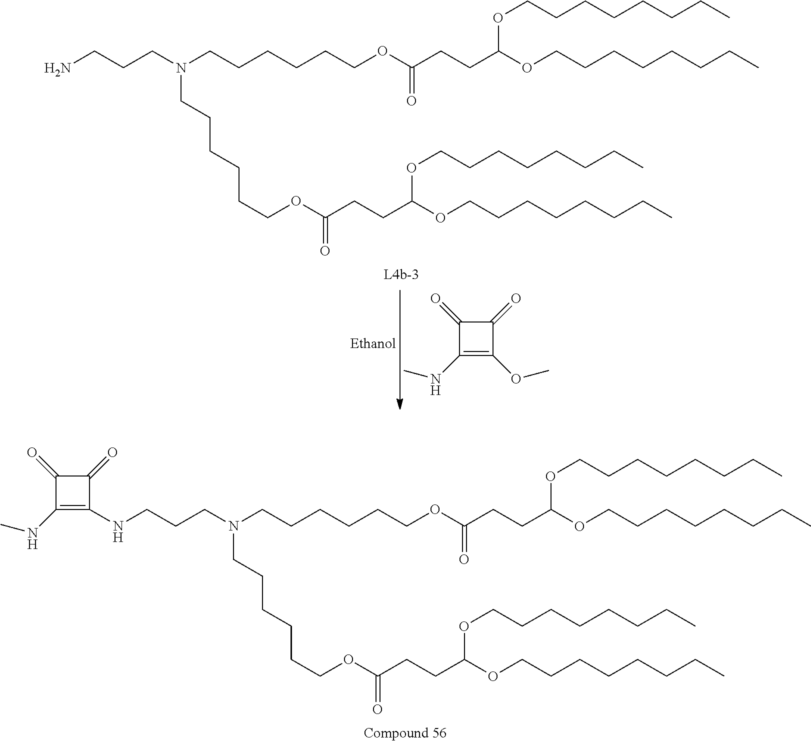

### Synthesis of ((3-((2-(methylamino)-3,4-dioxocyclobut-1-en-1-yl)amino)propyl)azanediyl) bis(hexane-6,1-diyl) bis(4,4-bis(octyloxy)butanoate) (Compound 56)

To a solution of L4b-3 (750 mg, 0.809 mmol) in ethanol (5 mL) was added 3-methoxy-4-(methylamino)cyclobut-3-ene-1,2-dione (171 mg, 1.214 mmol) at 0° C. and the reaction was allowed to room temperature and stirred for 24 h. Solvent was removed in vacuo, and crude material was purified by flash column chromatography (SiO₂: 0 to 10% MeOH/DCM (1% NH₄OH)) to yield Compound 56 as light yellow color sticky oil (110 mg, 13%). ¹H-NMR (300 MHz, CDCl₃) δ 4.48 (t, 2H), 4.05 (t, 4H), 3.72-3.68 (m, 1H), 3.60-3.52 (m, 4H), 3.43-3.36 (m, 4H), 3.29 (d, 3H), 2.92-2.60 (m, 3H), 2.38 (t, 4H), 1.95-1.86 (m, 6H), 1.68-1.46 (m, 18H), 1.40-1.24 (m, 53H), 0.89-0.85 (t, 12H). CIMS m/z 1036.9 [M+H]⁺ 1036.9. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.52 min, purity: 95.32%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=3.26 min, purity: 87.12%.

### Synthesis of ((3-((tert-butoxycarbonyl)amino)propyl)azanediyl)bis(hexane-6,1-diyl) bis(4,4-bis(octyloxy) butanoate) (Compound 60)

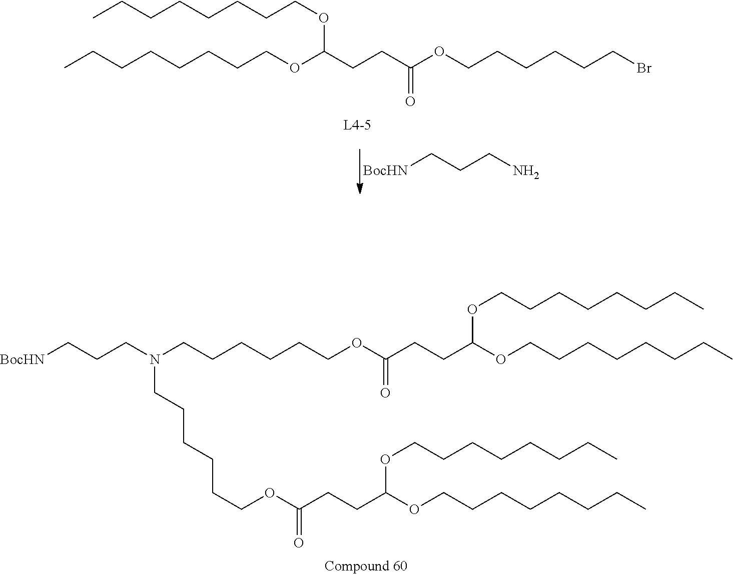 [SMILES: CCCCCCCCOC(CCC(=O)OCCCCCCBr)OCCCCCCCC ; CC(C)(C)OC(=O)NCCCN ; CCCCCCCCOC(CCC(=O)OCCCCCCN(CCCCCCOC(=O)CCC(OCCCCCCCC)OCCCCCCCC)CCCNC(=O)OC(C)(C)C)OCCCCCCCC]

### Synthesis of ((3-((tert-butoxycarbonyl)amino)propyl)azanediyl)bis(hexane-6,1-diyl) bis(4,4-bis(octyloxy) butanoate) (Compound 60)

To a solution of compound L4-5 (5.0 g, 9.88 mmol) in CH₃CN (5 mL) and CPME (5 mL) under nitrogen was added tert-butyl (3-aminopropyl)carbamate (939 mg, 4.94 mmol) followed by the addition of K₂CO₃ (4.09 g, 29.63 mmol) and KI (4.92 g, 29.63 mmol). The reaction mixture was heated at 85° C. for 18 h. After cooling to room temperature, the reaction mixture was filtered through Celite, washed with ethyl acetate, and the solvent was removed under vacuum to give the crude product which was purified by flash chromatography (SiO2:MeOH in DCM 0-10% gradient) to yield Compound 60 as colorless oil (4.0 g, 79%). ¹H-NMR (300 MHz, CDCl₃) δ; 4.49 (t, 2H), 4.05 (t, 4H), 3.60-3.52 (m, 4H), 3.43-3.36 (m, 4H), 3.15 (b, 2H), 2.50-2.25 (m, 10H), 1.95-1.88 (q, 4H), 1.67-1.16 (m, 76H), 0.87 (m, 12H); CIMS m/z [M+H]⁺ 1027.9; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.75 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.07 min, purity: >99%.

### Synthesis of Compound 51 ((4-hydroxybutyl)azanediyl)bis(pentane-5,1-diyl) bis(4,4-bis(octyloxy)butanoate)

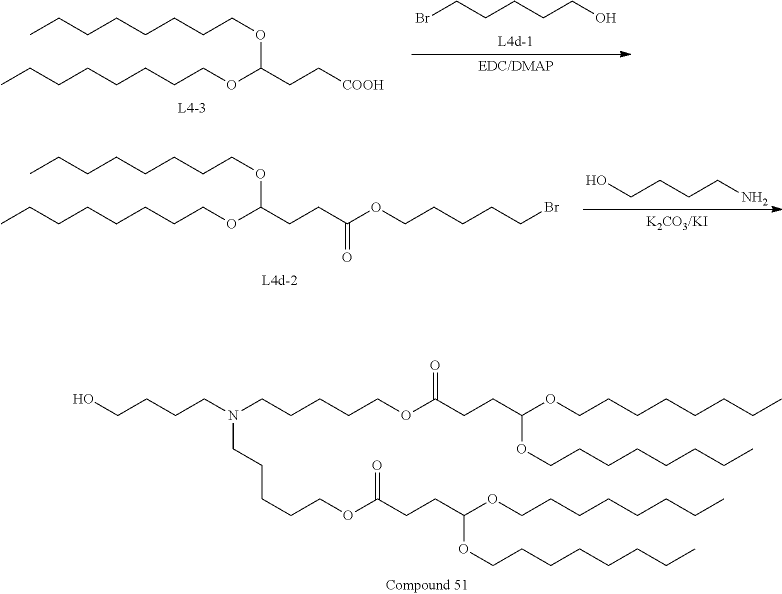 [SMILES: OCCCCCBr ; CCCCCCCCOC(CCC(=O)O)OCCCCCCCC ; CCCCCCCCOC(CCC(=O)OCCCCCBr)OCCCCCCCC ; NCCCCO ; CCCCCCCCOC(CCC(=O)OCCCCCN(CCCCO)CCCCCOC(=O)CCC(OCCCCCCCC)OCCCCCCCC)OCCCCCCCC]

### Synthesis of 5-bromopentyl 4,4-bis(octyloxy)butanoate (L4d-2)

To a solution of compound L4-3 (3.0 g, 8.70 mmol) in dichloromethane (60 ml) was added DMAP (1.06 mg, 8.70 mmol), EDC (6.67 g, 34.8 mmol) and L4d-1 (3.2 g, 19.1 mmol). The reaction mixture was stirred at room temperature for 12 h. The reaction mixture was evaporated under vacuum. The residue was dissolved in dichloromethane (100 mL) and washed with brine (80 mL×3). After drying over anhydrous Na₂SO₄, the solvent was evaporated, and the crude material was purified by column chromatography (330 g SiO₂: 0 to 30% Ethyl acetate in Hexane gradient) to obtain L4d-2 as colorless oil (2.6 g, 60%). ¹H-NMR (300 MHz, CDCl₃) δ 4.48 (t, 1H), 4.06 (t, 2H), 3.59-3.31 (m, 6H), 2.37 (t, 2H), 1.95-1.85 (m, 4H), 1.67-1.47 (m, 8H), 1.38-1.18 (m, 20H), 0.86 (t, 6H).

### Synthesis of ((4-hydroxybutyl)azanediyl)bis(pentane-5,1-diyl) bis(4,4-bis(octyloxy)butanoate) (Compound 51)

To a solution of compound L4d-2 (2.5 g, 5.1 mmol) in CPME (15 mL) and ACN (15 mL) under nitrogen was added 4-amino-1-butanol (200 mg, 2.24 mmol) followed by the addition of K₂CO₃ (1.2 g, 8.9 mmol) and KI (372 mg, 2.5 mmol). The reaction mixture was heated at 80° C. for 18 h. After cooling to room temperature, the reaction mixture was filtered through Celite, washed with ethyl acetate, and the solvent was removed under vacuum to give the crude product which was purified by flash chromatography. (80 g SiO₂: 0 to 30% Ethyl acetate in 5% triethylamine in hexane gradient) to obtain Compound 51 as colorless oil (0.8 g, 40%). ¹H-NMR (300 MHz, CDCl₃) δ 4.48 (t, 2H), 4.04 (t, 4H), 3.59-3.35 (m, 10H), 2.44-2.34 (m, 10H), 1.94-1.87 (m, 4H), 1.67-1.44 (m, 20H), 1.38-1.13 (m, 45H), 0.86 (t, 12H)); CIMS m/z [M+H]⁺ 914.7. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.4 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.9 min, purity: >97%.

### Synthesis of Compound 52 ((4-hydroxybutyl)azanediyl)bis(octane-8,1-diyl) bis(4,4-bis(octyloxy)butanoate)

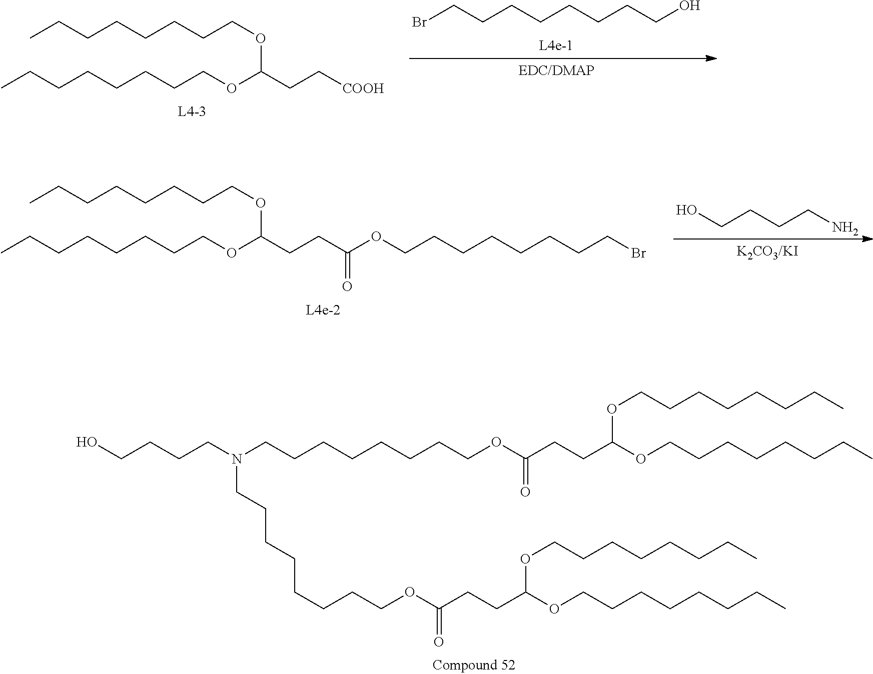 [SMILES: CCCCCCCCOC(CCC(=O)O)OCCCCCCCC ; OCCCCCCCCBr ; CCCCCCCCOC(CCC(=O)OCCCCCCCCBr)OCCCCCCCC ; NCCCCO ; CCCCCCCCOC(CCC(=O)OCCCCCCCCN(CCCCO)CCCCCCCCOC(=O)CCC(OCCCCCCCC)OCCCCCCCC)OCCCCCCCC]

### Synthesis of 8-bromooctyl 4,4-bis(octyloxy)butanoate (L4e-2)

To a solution of compound L4-3 (3.0 g, 8.70 mmol) in dichloromethane (60 mL) was added DMAP (1.06 mg, 8.70 mmol), EDCI (6.67 g, 34.8 mmol) and L4e-1 (4.0 g, 19.1 mmol). The reaction mixture was stirred at room temperature for 12 h. The reaction mixture was evaporated under vacuum. The residue was dissolved in dichloromethane (100 mL) and washed with brine (80 mL×3). After drying over anhydrous Na₂SO₄, the solvent was evaporated, and the crude was purified by column chromatography (330 g SiO₂: 0 to 30% Ethyl acetate in Hexane gradient) to obtain compound L4e-2 (3.3 g, 70%). ¹H-NMR (300 MHz, CDCl₃) δ 4.48 (t, 1H), 4.04 (t, 2H), 3.59-3.35 (m, 6H), 2.37 (t, 2H), 1.95-1.86 (m, 3H), 1.67-1.08 (m, 35H), 0.86 (t, 6H). CIMS m/z [M+H]⁺ 536.4.

### Synthesis of ((4-hydroxybutyl)azanediyl)bis(octane-8,I-diyl) bis(4,4-bis(octyloxy)butanoate) (Compound 52)

To a solution of compound L4e-2 (3.17 g, 5.9 mmol) in CPME (15 mL) and ACN (15 ml) under nitrogen was added 4-amino-1-butanol (230 mg, 2.5 mmol) followed by the addition of K₂CO₃ (1.4 g, 10.3 mmol) and KI (428 mg, 2.5 mmol). The reaction mixture was heated at 80° C. for 18 h. After cooling to room temperature, the reaction mixture was filtered through Celite, washed with ethyl acetate, and the solvent was removed under vacuum to give the crude product which was purified by flash chromatography. (80 g SiO₂: 0 to 30% Ethyl acetate in 5% triethylamine in hexane gradient) to give Compound 52 (1.2 g, 47%) as colorless oil. ¹H-NMR (300 MHz, CDCl₃) δ 4.49 (t, 2H), 4.03 (t, 4H), 3.59-3.35 (m, 10H), 2.43-2.34 (m, 10H), 1.94-1.87 (m, 4H), 1.63-1.42 (m, 18H), 1.37-1.18 (m, 58H), 0.86 (t, 12H); CIMS m/z [M+H]⁺ 998.8. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.8 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=14.8 min, purity: >87%.

### Synthesis of Compound 55 ((4-hydroxybutyl) azanediyl)bis(hexane-6,1-diyl) bis(6,6-bis(octyloxy) hexanoate)

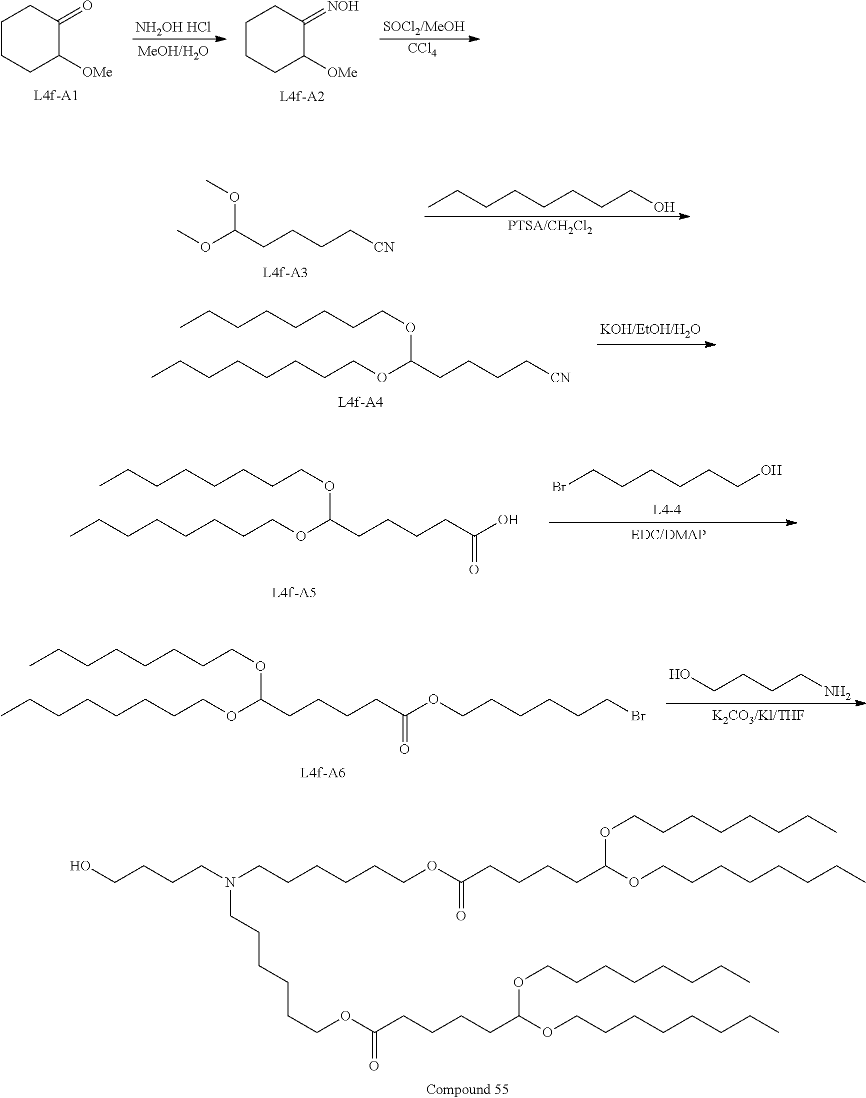

### Synthesis of (E)-2-methoxycyclohexan-1-one oxime (L4f-A2)

To a 5 mL microwave reactor tube L4f-A1 (1.0 g, 7.80 mmol), hydroxylamine hydrochloride (1.139 g, 16.38 mmol), and sodium acetate (1.34 g, 16.38 mmol) were added. To this was added a mixture of MeOH and Water (2:1, 2 mL) and the reaction mixture was sealed with the vial cap and heated in a CEM microwave reactor at 100° C. for 5 min. After completion of the reaction the solvent was removed under vacuum. The residue was partitioned between ethyl acetate/brine (3 times). The ethyl acetate layers were combined and dried over anhydrous magnesium sulphate. The ethyl acetate was removed under vacuum to give 940 mg (84%) crude product L4f-A2. This crude product was used for the next step reaction without further purification. ¹H NMR (300 MHz, CDCl₃): δ ppm 8.32 (bs, 1H), 3.72 (t, J=5.0 Hz, 1H), 3.27 (s, 3H), 3.10-3.25 (m, 1H), 2.10-2.01 (m, 2H), 1.81-1.30 (m, 6H) MS (CI): m/z [M+H]⁺ 143.2.

### Synthesis of 6,6-dimethoxyhexanenitrile (L4f-A3)

To a 100 mL of three neck flask L4f-A2 (5.7 g, 44.47 mmol) was added carbon tetrachloride (30 mL). The flask was flushed with nitrogen and cooled to 0° C. To this flask was added thionyl chloride (4.64 g, 2.89 ml, 66.70 mmol) dissolved in carbon tetrachloride (15 mL) dropwise over 10 min. The reaction was maintained at same temperature and stirred for 5 min. Anhydrous methanol (20 mL) was added dropwise to the reaction. The reaction was allowed to stir at 0-5° C. for 1 h. After completion of reaction, the reaction contents were poured into a 1000 mL round bottom flask containing 30 g of sodium bicarbonate and brine (5 mL). The reaction contents were allowed to neutralize for 5 min. Solvents were evaporated under vacuum. The residue was partitioned between ethyl acetate/brine (3 times), the ethyl acetate layers were combined, and passed through celite. The ethyl acetate layer was dried over anhydrous magnesium sulphate. The ethyl acetate was removed under vacuum and residue was kept in high vacuum to give crude product L4f-A3 (4.07 g, 58%). This crude was used for the next step without further purification. ¹H NMR (300 MHz, CDCl₃): δ ppm 4.35 (t, J=5.0 Hz, 1H), 3.32 (s, 6H), 2.31 (t, J=6.0 Hz, 2H), 1.70-1.51 (m, 6H); MS (CI): m/z [M+H]⁺ 157.3.

### Synthesis of 6,6-bis(octyloxy)hexanenitrile (L4f-A4)

To a 100 mL round bottom flask was added L4f-A3 (2.0 g, 12.72 mmol) and octanol (9.94 g, 76.33 mmol). To this was added Pyridinium p-toluene sulfonate (480 mg, 1.91 mmol) and the reaction mixture was stirred at 120° C. for 2 h. After completion of the reaction the contents were loaded on to a 120 g flash silica column and purified by flash chromatography (SiO₂: ethyl acetate/hexane 0-20%) to get Compound L4f-A4 (2.99 g, 67%) as slightly yellow oil. ¹H NMR (300 MHz, CDCl₃): δ ppm 4.45 (t, J=6.0 Hz, 1H), 3.57-3.53 (m, 2H), 3.42-3.37 (m, 2H), 2.33 (t, J=7.0 Hz, 2H), 1.62-1.50 (m, 6H), 1.41-1.16 (m, 24H), 0.89-0.84 (m, 6H); MS (CI): m/z [M+H]⁺ 354.5.

### Synthesis of 6,6-bis(octyloxy)hexanoic acid (L4f-A5)

To a 250 mL round bottom flask L4f-A4 (2.94 g, 8.31 mmol) and KOH (3.86 g, 68.815 mmol) were added. To this was added a mixture of ethanol and water (1:1, 80 mL) and the reaction mixture was refluxed at 110° C. for 30 h. After completion of the reaction the solvent was removed under vacuum. Residual paste was mixed with 100 g ice and acidified to pH 5 using 1M HCl. This mixture was partitioned in ethyl acetate: water. To this was added brine (100 mL). This mixture was filtered to remove any emulsion formed. The ethyl acetate layer separated well after filtration. The product was extracted with ethyl acetate three times. The combined ethyl acetate layers were dried over anhydrous sodium sulphate. The ethyl acetate was removed under vacuum to give crude compound L4f-A5 (2.45 g, 79%) as slightly yellow oil. ¹H NMR (300 MHz, CDCl₃): (5 ppm 4.44 (t, J=3.0 Hz, 1H), 3.55-3.50 (m, 2H), 3.42-3.36 (m, 2H), 2.33 (m, 2H), 1.59-1.16 (m, 30H), 0.89-0.84 (m, 6H); MS (CI): m/z [M+H]⁺ 373.6.

### Synthesis of 6-bromohexyl 6,6-bis(octyloxy)hexanoate (L4f-A6)

To a 250 mL round bottom flask L4f-A5 (2.0 g, 5.37 mmol), EDC (4.12 g, 21.47 mmol), DMAP (0.656 g, 5.37 mmol) were added. To this was added 6-bromohexanol (1.94 g, 10.73 mmol) along with dichloromethane (60 mL) and the reaction mixture was stirred at room temperature for 2 h. After completion of the reaction about 30 g of flash silica was added and the contents were stirred to yield a uniform mixture. Solvent was removed from this mixture under vacuum. The residue was loaded on to an empty flash cartridge, which was then attached to flash purification system loaded with 80 g flash silica column and was purified by flash chromatography (SiO₂: ethyl acetate/hexane 0-20%) to get Compound L4f-A6 (1.935 g, 67%) as slightly yellow oil. ¹H NMR (300 MHz, CDCl₃): o ppm 4.49 (t, J=6.0 Hz, 1H), 4.05 (t, J=6.0 Hz, 2H), 3.56-3.54 (m, 2H), 3.40-3.37 (m, 4H), 2.36 (t, J=7.0 Hz, 2H), 1.94-1.85 (m, 4H), 1.58-1.23 (m, 36H), 0.89-0.87 (m, 6H); MS (CI): m/z [M+H]⁺ 535.4.

### Synthesis of ((4-hydroxybutyl) azanediyl)bis(hexane-6,1-diyl) bis(6,6-bis(octyloxy) hexanoate) (Compound 55)

To a 40 mL microwave reactor tube L4f-A6 (1.56 g, 2.912 mmol), 4-amino-1-butanol (116.8 mg, 1.31 mmol), potassium carbonate (1.207 g, 8.74 mmol) and potassium iodide (725.1 mg, 4.368 mmol) were added. To this was added a mixture of CPME and ACN (1:1, 5 mL) and the reaction mixture was sealed and heated in a CEM microwave reactor at 100° C. for 6 h. After completion of the reaction, the contents were transferred to a 500 mL round bottom flask containing about 40 g of Flash silica (this silica was stirred in 300 mL 5% triethylamine in hexane for 10 min before use) and the contents were stirred well to get a uniform mixture. Solvent was removed from this mixture under vacuum. The residue was loaded on to an empty flash cartridge, which was then attached to flash purification system loaded with a 80 g size prepacked flash silica column (the flash column was equilibrated with 10% triethylamine in hexane for 15 min at a flow rate of 80 mL per min before use) and was purified by flash chromatography (SiO₂: ethyl acetate/hexane 0-50%) to get Compound 55 (520 mg, 18%). ¹H NMR (300 MHz, CDCl₃): δ ppm 6.56 (bs, 1H), 4.44 (t, J=6.0 Hz, 2H), 4.03 (t, J=7.0 Hz, 4H), 3.56-3.36 (m, 10H), 2.42-2.28 (m, 10H), 1.95-1.88 (m, 4H), 1.64-1.20 (m, 70H), 0.89-0.86 (m, 12H); MS (CI): m/z [M+H]⁺ 998.6. Analytical HPLC column: Agilent Zorbax SB-C18, 5 μm, 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: 96.78%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.1 min, purity: >99%.

### Synthesis of Compound 58 ((4-hydroxybutyl) azanediyl)bis(hexane-6,1-diyl) bis(4,4-bis(hexyloxy) butanoate)

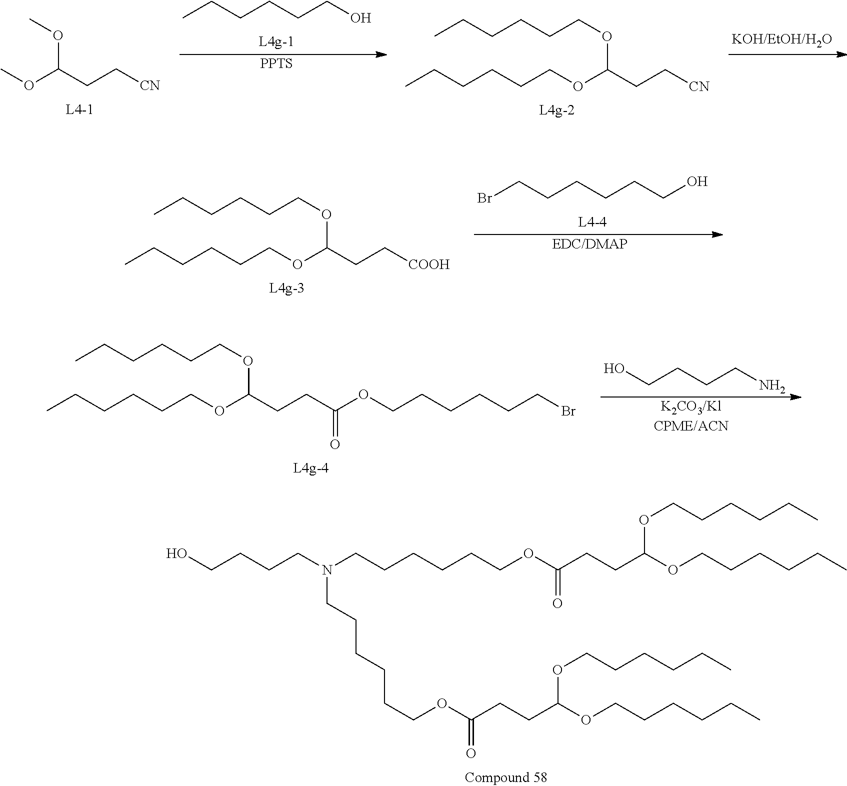

### Synthesis of 4,4-bis(hexyloxy)butanenitrile (L4g-2)

To a 250 mL round bottom flask L4-1 (10.0 g, 77.42 mmol) and L4g-1 (30.11 g, 294.6 mmol) were added. To this was added Pyridinium p-toluenesulfonate (0.5 g, 1.989 mmol) and the reaction mixture was stirred at 125° C. for 26 h. After completion of the reaction the contents were loaded on to a 220 g flash silica column and was purified by flash chromatography (SiO₂: ethyl acetate/hexane 0-10%) to get Compound L4g-2 (16.4 g, 79%) as slightly yellow oil. ¹H NMR (300 MHz, CDCl₃): δ ppm 4.54 (t, J=6.0 Hz, 1H), 3.63-3.55 (m, 2H), 3.46-3.38 (m, 2H), 2.43 (t, J=6.0 Hz, 2H), 1.95-1.92 (m, 2H), 1.56-1.32 (m, 4H), 1.32-1.29 (m, 12H), 0.87-0.85 (m, 6H); MS (CI): m/z [M+H]⁺ 270.3.

### Synthesis of 4,4-bis(hexyloxy)butanoic acid (L4g-3)

To a 250 mL round bottom flask L4g-2 (6.8 g, 25.24 mmol) and KOH (4.25 g, 75.71 mmol) were added. To this was added ethanol (50 mL) and water (50 mL) and the reaction mixture was refluxed at 110° C. for 18 h. After completion of the reaction the solvent was removed under vacuum. Residual paste was mixed with 100 g ice and acidified to pH 5 using 4M HCl. This mixture was partitioned in ethyl acetate and water. To this was added brine (100 mL). The ethyl acetate layer was separated. The product was extracted with ethyl acetate three times. The combined ethyl acetate layers were dried over anhydrous sodium sulphate. The ethyl acetate was removed under vacuum to give crude product. This crude was loaded on to a 220 g flash silica column and was purified by flash chromatography (SiO₂: ethyl acetate/hexane 0-10%) to get Compound L4g-3 (6.9 g, 94%) as slightly yellow oil. ¹H NMR (300 MHz, CDCl₃): δ ppm 4.50 (t, J=6.0 Hz, 1H), 3.59-3.56 (m, 2H), 3.42-3.39 (m, 2H), 2.44-2.42 (m, 2H), 1.95-1.93 (m, 2H), 1.59-1.28 (m, 24H), 0.87-0.85 (m, 6H); MS (CI): m/z [M+H]⁺ 289.4.

### Synthesis of 6-bromohexyl 4,4-bis(hexyloxy)butanoate Compound (L4g-4)

To a 250 mL round bottom flask L4g-3 (3.0 g, 10.40 mmol), EDC (2.99 g, 15.60 mmol), DMAP (0.25 g, 2.08 mmol), and N, N-diisopropylethylamine (5.37 g, 41.65 mmol) were added in DCM (40 mL). To this was added 6-bromohexanol (2.45 g, 13.52 mmol) and the reaction mixture was stirred at room temperature for 3 days. After completion of the reaction the solvent was removed under vacuum. Residual paste was partitioned in ethyl acetate and water. It gave a milky emulsion. To this was added brine (100 mL). The milky emulsion was acidified to pH4 using 4M HCl. The ethyl acetate layer separated. The product was extracted with ethyl acetate three times. The combined ethyl acetate layers were washed with bicarbonate and dried over anhydrous sodium sulphate. The ethyl acetate was removed under vacuum and residue was loaded on to a 220 g flash silica column and was purified by flash chromatography (SiO₂: ethyl acetate/hexane 0-20%) to get Compound L4g-4 (1.0 g, 22%) as slightly yellow oil. ¹H NMR (300 MHz, CDCl₃): δ ppm 4.50 (t, J=6.0 Hz, 1H), 4.05 (t, J=7.0 Hz, 2H), 3.58-3.35 (m, 6H), 2.37-2.34 (m, 2H), 1.93-1.90 (m, 2H), 1.59-1.28 (m, 24H), 0.87-0.85 (m, 6H); MS (CI): m/z [M+H]⁺ 452.4.

### Synthesis of ((4-hydroxybutyl) azanediyl)bis(hexane-6,1-diyl) bis(4,4-bis(hexyloxy) butanoate) (Compound 58)

To a 250 mL round bottom flask L4g-4 (2.2 g, 4.87 mmol), 4-amino-1-butanol (0.195 g, 2.19 mmol), KI (1.213 g, 7.31 mmol) and potassium carbonate (2.02 g, 14.618 mmol) were added. To this was added anhydrous acetonitrile (10 mL) along with cyclopentylmethyl ether (CPME) (10 mL) and the reaction mixture was stirred under reflux at 90° C. for 36h. After completion of the reaction about 40 g of Flash silica (this silica neutralized by stirring in 300 mL of 5% triethylamine in hexane for 10 min before use) was added and the contents were stirred well to get a uniform mixture. Solvent was removed from this mixture under vacuum. The residue was loaded on to an empty flash cartridge, which was then attached to flash purification system loaded with 80 g size prepacked flash silica column (The flash column was equilibrated with 10% triethylamine in hexane for 15 min at a flow rate of 80 mL per min before use) and was purified by flash chromatography (SiO₂: ethyl acetate/hexane 0-50%) to get Compound 58 (1.1 g, 28%). ¹H NMR (300 MHz, CDCl₃): δ ppm 4.50 (t, J=6.0 Hz, 2H), 4.05 (t, J=7.0 Hz, 4H), 3.58-3.34 (m, 10H), 2.42-2.34 (m, 10H), 1.95-1.88 (m, 4H), 1.64-1.28 (m, 54H), 0.89-0.85 (m, 12H); MS (CI): m/z [M+H]⁺ 831.3. Analytical HPLC column: Agilent Zorbax SB-C18, 5 μm, 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20+2° C., detector: CAD, t_{R}=13.1 min, purity: >99%.

### Synthesis of Compound 57 ((4-hydroxybutyl)azanediyl)bis(hexane-6,1-diyl) bis(4,4-bis(decyloxy) butanoate)

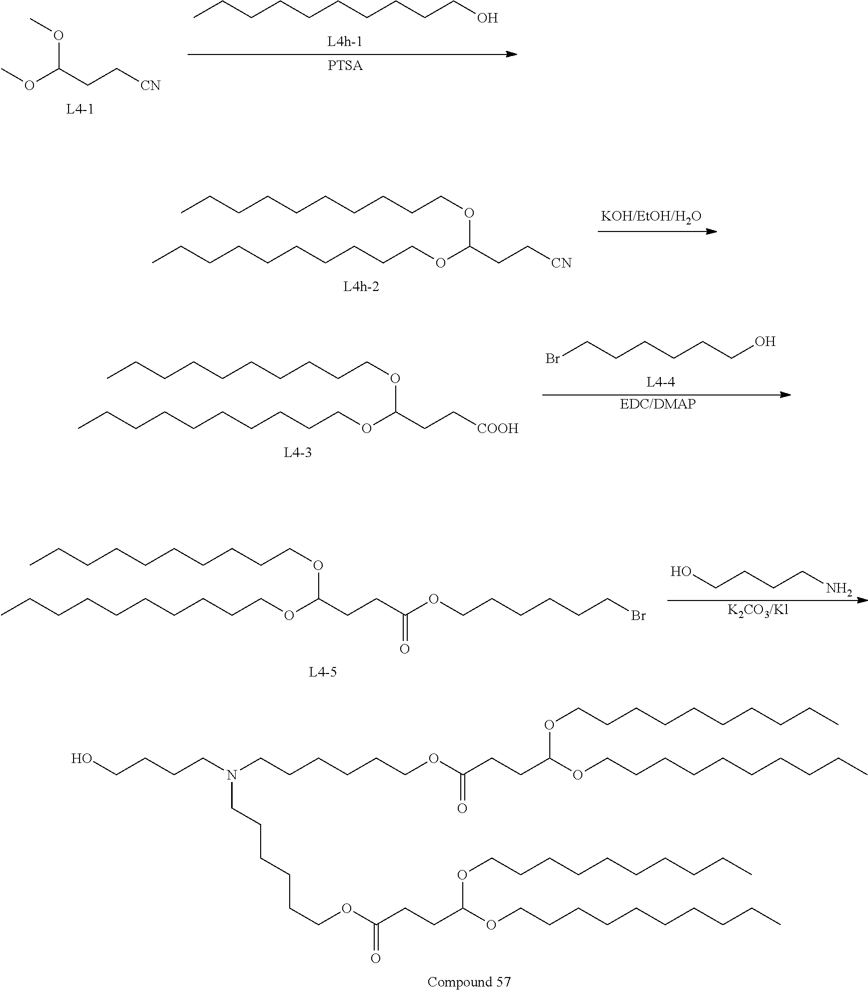

### Synthesis of 4,4-bis(decyloxy)butanenitrile (L4h-2)

To a 100 mL round bottom flask L4-1 (5.0 g, 31.58 mmol) and L4h-1 (15.05 g, 95.11 mmol) were added. To this was added Pyridinium p-toluenesulfonate (0.26 g, 1.015 mmol) and the reaction mixture was stirred at 125° C. for 26 h. After completion of the reaction the contents were loaded on to a 220 g flash silica column and purified by flash chromatography (SiO₂: ethyl acetate/hexane 0-10%) to get Compound L4h-2 (8.96 g, 61%) as slightly yellow oil. ¹H NMR (300 MHz, CDCl₃): δ ppm 4.54 (t, J=6.0 Hz, 1H), 3.63-3.55 (m, 2H), 3.46-3.38 (m, 2H), 2.43 (t, J=6.0 Hz, 2H), 1.95-1.92 (m, 2H), 1.56-1.32 (m, 4H), 1.32-1.29 (m, 28H), 0.87-0.85 (m, 6H); MS (CI): m/z [M+H]⁺ 381.4.

### Synthesis of 4,4-bis(decyloxy)butanoic acid (L4-3)

To a 500 mL round bottom flask L4h-2 (8.9 g, 23.47 mmol) and KOH (3.95 g, 70.43 mmol) were added. To this was added Ethanol (50 mL) and Water (50 mL). The reaction mixture was refluxed at 110° C. for 18 h. After completion of the reaction the solvent was removed under vacuum. Residual paste was mixed with 100 g ice and acidified to pH 5 using 4M HCl. This mixture was partitioned in ethyl acetate: water. To this was added brine (100 mL). The ethyl acetate layer was separated. The product was extracted with ethyl acetate three times. The combined ethyl acetate layers were dried over anhydrous sodium sulphate. The ethyl acetate was removed under vacuum to give crude product. This crude was loaded on to a 220 g flash silica column and was purified by flash chromatography (SiO₂: ethyl acetate/hexane 0-10%) to get Compound L4-3 (6.8 g, 73%) as slightly yellow oil. ¹H NMR (300 MHz, CDCl₃): δ ppm 4.52 (t, J=6.0 Hz, 1H), 3.59-3.53 (m, 2H), 3.42-3.38 (m, 2H), 2.46-2.42 (m, 2H), 1.95-1.93 (m, 2H), 1.59-1.28 (m, 28H), 0.89-0.84 (m, 6H); MS (CI): m/z [M+H]⁺ 401.3.

### Synthesis of 6-bromohexyl 4,4-bis(decyloxy)butanoate (L4-5)

To a 250 mL round bottom flask L4-3 (2.0 g, 4.992 mmol), EDC (3.83 g, 19.96 mmol), DMAP (0.61 g, 5.0 mmol) were added. To this was added 6-bromohexanol (1.81 g, 9.98 mmol) along with dichloromethane (80 mL) and the reaction mixture was stirred at room temperature for 2 h. After completion of the reaction about 40 g of Flash silica was added and the contents were stirred well to get a uniform mixture. Solvent was removed from this mixture under vacuum. The residue was loaded on to an empty flash cartridge, which was then attached to a flash purification system loaded with 80 g flash silica column and was purified by flash chromatography (SiO₂: ethyl acetate/hexane 0-10%) to get Compound L4-5 (2.18 g, 78%) as slightly yellow oil. ¹H NMR (300 MHz, CDCl₃): δ ppm 4.49 (t, J=6.0 Hz, 1H), 4.07 (t, J=6.0 Hz, 2H), 3.57-3.53 (m, 2H), 3.40-3.37 (m, 2H), 2.37 (t, J=7.0 Hz, 2H), 1.93-1.83 (m, 4H), 1.56-1.24 (m, 38H), 0.89-0.84 (m, 6H); MS (CI): m/z [M+H]⁺ 563.7.

### Synthesis of ((4-hydroxybutyl)azanediyl)bis(hexane-6,1-diyl) bis(4,4-bis(decyloxy) butanoate) (Compound 57)

To a 250 mL round bottom flask L4-5 (2.1 g, 3.725 mmol), 4-amino-1-butanol (0.149 g, 1.676 mmol), KI (0.93 g, 5.588 mmol) and potassium carbonate (1.54 g, 11.176 mmol) were added. To this was added anhydrous acetonitrile (15 mL) along with cyclopentylmethyl ether (CPME) (15 mL) and the reaction mixture was stirred under reflux at 90° C. for 36 h. After completion of the reaction about 40 g of Flash silica (this silica neutralized by stirring in 5% triethylamine in hexane (300 mL) for 10 min before use) was added and the contents were stirred well to get a uniform mixture. Solvent was removed from this mixture under vacuum. The residue was loaded on to an empty flash cartridge, which was then attached to flash purification system loaded with 80 g size prepacked flash silica column (The flash column was equilibrated with 10% triethylamine in hexane for 15 min at a flow rate of 80 mL per min before use) and was purified by flash chromatography (SiO₂: ethyl acetate/hexane 0-30%) to get Compound 57 (1.lg, 28%). ¹H NMR (300 MHz, CDCl₃): δ ppm 4.48 (t, J=6.0 Hz, 2H), 4.05 (t, J=7.0 Hz, 4H), 3.58-3.36 (m, 10H), 2.44-2.34 (m, 10H), 1.92-1.90 (m, 2H), 1.64-1.20 (m, 86H), 0.89-0.84 (m, 12H); MS (CI): m/z [M+H]⁺ 1054.7. Analytical HPLC column: Agilent Zorbax SB-C18, 5 μm, 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.1 min, purity: >99%.

### Synthesis of Compound 59 ((4-hydroxybutyl)azanediyl)bis(hexane-6,1-diyl) bis(6,6-bis(hexyloxy) hexanoate)

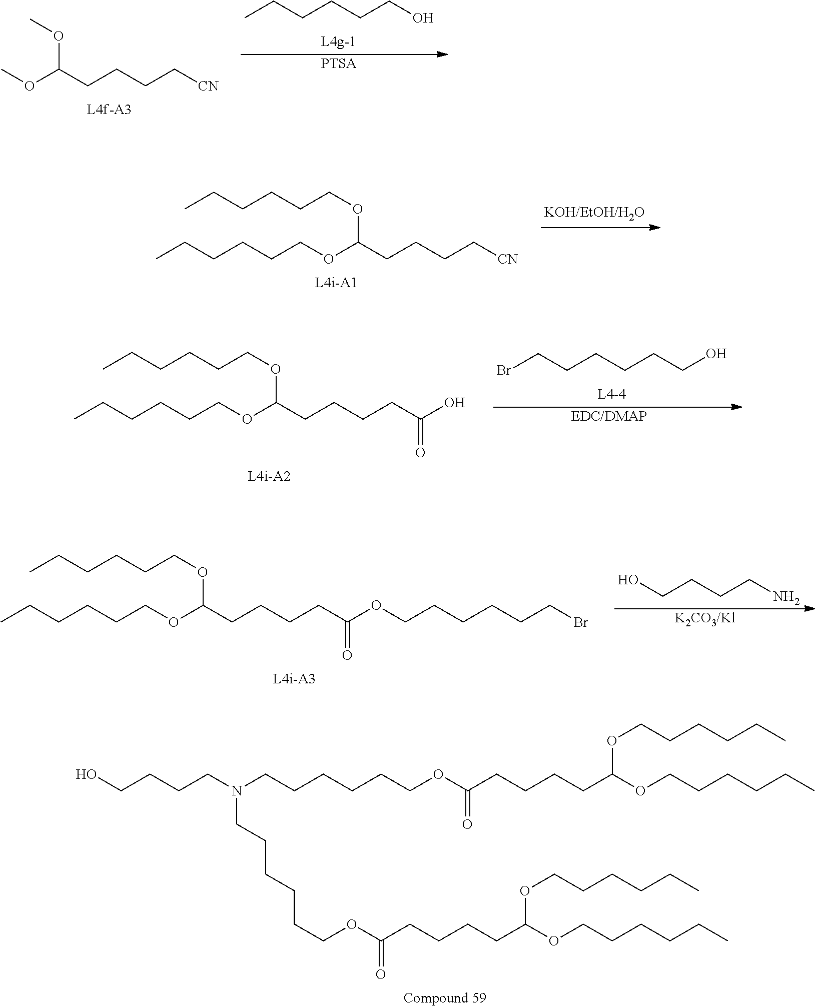

### Synthesis of 6,6-bis(hexyloxy)hexanenitrile (L4i-A1)

To a 100 mL round bottom flask L4f-A3 (2.0 g, 12.72 mmol) and 1-hexanol (7.798 g, 76.33 mmol) were added. To this was added Pyridinium p-toluene sulfonate (480 mg, 1.91 mmol) and the reaction mixture was stirred at 120° C. for 2 h. After completion of the reaction the contents were loaded on to a 120 g flash silica column and was purified by flash chromatography (SiO₂: ethyl acetate/hexane 0-20%) to get Compound L4i-A1 (2.45 g, 65%) as slightly yellow oil. ¹H NMR (300 MHz, CDCl₃): δ ppm 4.45 (t, J=6.0 Hz, 1H), 3.57-3.53 (m, 2H), 3.42-3.37 (m, 2H), 2.33 (t, J=7.0 Hz, 2H), 1.62-1.50 (m, 6H), 1.41-1.16 (m, 16H), 0.89-0.84 (m, 6H); MS (CI): m/z [M+H]⁺ 298.5.

### Synthesis of 6,6-bis(hexyloxy)hexanoic acid (L4i-A2)

To a 250 mL round bottom flask L4i-A1 (2.45 g, 8.24 mmol) and KOH (1.386 g, 24.70 mmol) were added. To this was added mixture of 1:1 Ethanol: Water (80 mL) and the reaction mixture was refluxed at 110° C. for 30 h. After completion of the reaction the solvent was removed under vacuum. Residual paste was mixed with 100 g ice and acidified to pH 5 using 1M HCl. This mixture was partitioned in ethyl acetate: water. To this was added brine (100 mL). This mixture was filtered to remove any emulsion formed. The ethyl acetate layer separated well after filtration. The product was extracted with ethyl acetate three times.

The combined ethyl acetate layers were dried over anhydrous sodium sulphate. The ethyl acetate was removed under vacuum to give L4i-A2 (2.07 g, 79%). as slightly yellow oil. ¹H NMR (300 MHz, CDCl₃): δ ppm 4.45 (t, J=7.0 Hz, 1H), 3.56-3.50 (m, 2H), 3.42-3.37 (m, 2H), 2.33 (t, J=7.0 Hz, 2H), 1.69-1.20 (m, 22H), 0.89-0.84 (m, 6H); MS (CI): m/z [M+H]⁺ 316.4.

### Synthesis of 6-bromohexyl 6,6-bis(hexyloxy)hexanoate (L4i-A3)

To a 250 mL round bottom flask L4i-A2 (2.07 g, 6.54 mmol), EDC (5.015 g, 26.16 mmol), DMAP (0.799 g, 6.54 mmol) were added. To this was added 6-bromohexanol (2.368 g, 13.08 mmol) along with dichloromethane (60 mL) and the reaction mixture was stirred at room temperature for 120 min. The reaction was monitored by TLC and using PMA staining. After completion of the reaction about 30g of flash silica was added and the contents were stirred well to get a uniform mixture. Solvent was removed from this mixture under vacuum. The residue was loaded on to an empty flash cartridge, which was then attached to flash purification system loaded with 80 g flash silica column and was purified by flash chromatography (SiO₂: ethyl acetate/hexane 0-20%) to get Compound L4i-A3 (2.35 g, 75%) as slightly yellow oil. ¹H NMR (300 MHz, CDCl₃): δ ppm 4.44 (t, J=6.0 Hz, 1H), 4.05 (t, J=6.0 Hz, 2H), 3.56-3.52 (m, 2H), 3.42-3.36 (m, 4H), 2.29 (t, J=7.0 Hz, 2H), 1.88-1.80 (m, 4H), 1.62-1.27 (m, 28H), 0.89-0.85 (m, 6H); MS (CI): m/z [M+H]⁺ 479.5.

### Synthesis of ((4-hydroxybutyl)azanediyl)bis(hexane-6,1-diyl) bis(6,6-bis(hexyloxy) hexanoate) (Compound 59)

To a 40 mL microwave reactor tube L4f-A3 (2.3 g, 4.29 mmol), 4-amino-1-butanol (172.2 mg, 1.93 mmol), potassium carbonate (1.78 g, 12.88 mmol) and potassium iodide (1.07 g, 6.44 mmol) were added. To this was added a mixture of CPME and ACN (1:1, 5 mL) and the reaction mixture was sealed with cap and heated in CEM microwave reactor at 100° C. for 6 h. After completion of the reaction, the contents were transferred to a 500 mL round bottom flask containing about 40 g of Flash silica (this silica was stirred in of 10% triethylamine in hexane (300 mL) for 10 min before use) was added and the contents were stirred well to get a uniform mixture. Solvent was removed from this mixture under vacuum. The residue was loaded on to an empty flash cartridge, which was then attached to flash purification system loaded with 80 g size prepacked flash silica column (the flash column was equilibrated with 10% triethylamine in hexane for 15 min at a flow rate of 80 mL per min before use) and was purified by flash chromatography (SiO₂: ethyl acetate/hexane 0-50%) to get Compound 59 (1.1 g, 29%). ¹H NMR (300 MHz, CDCl₃): δ ppm 6.67 (bs, 1H), 4.44 (t, J=6.0 Hz, 2H), 4.03 (t, J=7.0 Hz, 4H), 3.56-3.36 (m, 10H), 2.44-2.28 (m, 10H), 1.63-1.28 (m, 64H), 0.89-0.84 (m, 12H); MS (CI): m/z [M+H]⁺ 886.4. Analytical HPLC column: Agilent Zorbax SB-C18, 5 μm, 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >96.11%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.1 min, purity: >99%.

### Synthesis of Compound 53 ((4-hydroxybutyl)azanediyl)bis(octane-8,1-diyl) bis(4,4-bis(hexyloxy) butanoate)

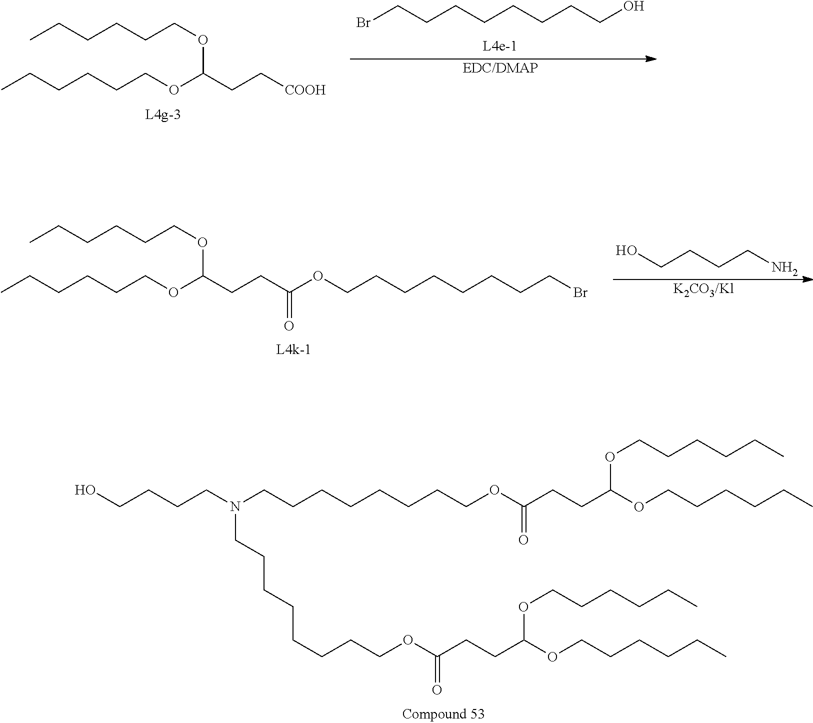

### Synthesis of 8-bromooctyl 4,4-bis(hexyloxy)butanoate (L4k-1)

To a 250 mL round bottom flask L4g-3 (1.6 g, 5.547 mmol), EDC (4.25 g, 22.189 mmol), DMAP (0.68 g, 5.547 mmol) were added. To this was added 6-bromooctanol (2.32 g, 11.095 mmol) along with dichloromethane (80 mL) and the reaction mixture was stirred at room temperature for 120 min. After completion of the reaction about 40g of flash silica was added and the contents were stirred well to get a uniform mixture. Solvent was removed from this mixture under vacuum. The residue was loaded on to an empty flash cartridge, which was then attached to flash purification system loaded with 80 g flash silica column and was purified by flash chromatography (SiO₂: ethyl acetate/hexane 0-10%) to get Compound L4k-1 (2.0 g, 75%) as slightly yellow oil. ¹H NMR (300 MHz, CDCl₃): δ ppm 4.49 (t, J=6.0 Hz, 1H), 4.05 (t, J=6.0 Hz, 2H), 3.55-3.54 (m, 2H), 3.40-3.37 (m, 4H), 2.37 (t, J=7.0 Hz, 2H), 1.93-1.84 (m, 4H), 1.57-1.24 (m, 28H), 0.89-0.87 (m, 6H); MS (CI): m/z [M+H]⁺ 479.5.

### Synthesis of ((4-hydroxybutyl)azanediyl)bis(octane-8,1-diyl) bis(4,4-bis(hexyloxy) butanoate) (Compound 53)

To a 250 mL round bottom flask L4k-1 (2.0 g, 4.17 mmol), 4-amino-1-butanol (0.167 g, 1.87 mmol), KI (1.04 g, 6.25 mmol) and potassium carbonate (1.73 g, 12.51 mmol) were added. To this was added anhydrous ACN (10 mL) along with cyclopentylmethyl ether (CPME) (10 mL) and the reaction mixture was stirred under reflux at 90° C. for 36 h. After completion of the reaction about 40 g of Flash silica (this silica was stirred in of 5% triethylamine in hexane (300 mL) for 10 min before use) was added and the contents were stirred well to get a uniform mixture. Solvent was removed from this mixture under vacuum. The residue was loaded on to an empty flash cartridge, which was then attached to flash purification system loaded with 80 g size prepacked flash silica column (the flash column was equilibrated with 10% triethylamine in hexane for 15 min at a flow rate of 80 mL per min before use) and was purified by flash chromatography (SiO₂: ethyl acetate/hexane 0-50%) to get Compound 53 (1.0 g, 27%). ¹H NMR (300 MHz, CDCl₃): δ ppm 4.48 (t, J=6.0 Hz, 2H), 4.03 (t, J=7.0 Hz, 4H), 3.58-3.37 (m, 10H), 2.44-2.34 (m, 10H), 1.95-1.88 (m, 4H), 1.64-1.20 (m, 60H), 0.89-0.85 (m, 12H); MS (CI): m/z [M+H]⁺ 886.3. Analytical HPLC column: Agilent Zorbax SB-C18, 5 μm, 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20+2° C., detector: CAD, t_{R}=13.1 min, purity: >99%.

### Synthesis of Compound 19 (4-(bis(7-((dioctylamino)oxy)-7-oxoheptyl)amino)butan-1-ol)

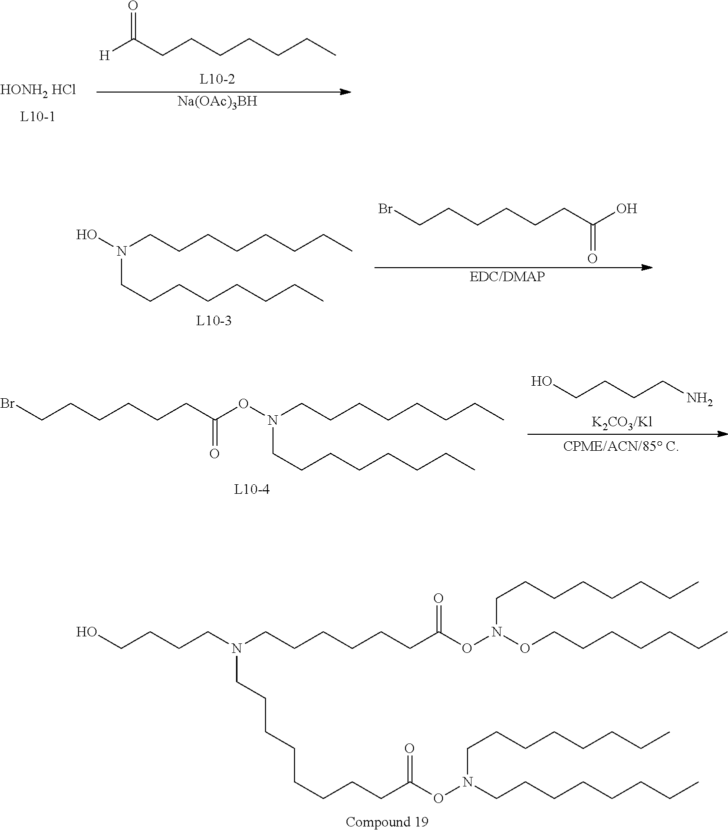

### Synthesis of N,N-dioctylhydroxylamine (L10-3)

A mixture of hydroxylamine hydrochloride L10-1 (1.75 g, 25 mmol), 1-octanal L10-2 (6.4 g, 50 mmol) and Et₃N (3.5 mL, 25 mmol) in dichloroethane (75 mL) was stirred at room temperature under nitrogen atmosphere to form a clear solution. NaBH(OAc)₃ (15.5 g, 75 mmol) was added in portions and the resulting mixture was stirred for 20 h at room temperature. Sat. aq. sodium bicarbonate solution was slowly added to the reaction mixture until no bubbles were produced. The resulting phases were separated. The aqueous phase was extracted again with DCM (150 mL). Combined organic extracts were dried over anhydrous Na₂SO₄. Filtration and concentration provided crude material which was purified by flash column chromatography (SiO₂: 0 to 100% EtOAc/Hexanes) to yield L10-3 (2.9 g, 53%) as white solid; ¹H-NMR (300 MHz, CDCl₃) δ 6.31 (s, bs, 1H), 2.63 (t, J=4.7 Hz, 4H), 1.70-1.49 (m, 4H), 1.43-1.15 (m, 20H), 0.87 (t, J=6.6 Hz, 6H). CIMS m/z [M+H]⁺ 258.2.

### Synthesis of O-(7-bromoheptanoyl)-N, N-dioctylhydroxylamine (L10-4)

To a solution of L10-3 (2.8 g, 11 mmol) in DCM (70 mL) was added 7-bromoheptanoic acid (2.8 g, 13 mmol) followed by DIPEA (4 mL, 22 mmol), DMAP (1.4 g, 11 mmol) and EDC (4.3 g, 22 mmol). The resulting mixture was stirred at room temperature under nitrogen atmosphere for 22 h. The reaction mixture was diluted with DCM (70 mL) and washed with saturated NaHCO₃ aqueous solution (50 mL), water (50 mL) and brine (50 mL). The organic phase was dried over anhydrous Na₂SO₄. Filtration and concentration provided crude material which was purified by flash column chromatography (SiO₂: 0 to 100% ethyl acetate in hexane gradient) to yield L10-4 as colorless oil (1.9 g, 38%). ¹H-NMR (300 MHz, CDCl₃) δ 3.40 (t, J=7.0 Hz, 2H), 2.79 (t, J=7.7 Hz, 4H), 2.28 (t, J=7.7 Hz, 2H), 1.91-1.15 (m, 34H), 0.86 (t, J=6.9 Hz, 6H). CIMS m/z [M+H]⁺ 448/450.

### Synthesis of 4-(bis(7-((dioctylamino)oxy)-7-oxoheptyl)amino)butan-1-ol (Compound 19)

A solution of L10-4 (650 mg, 1.4 mmol), 4-amino-1-butanol (56 mg, 0.6 mmol) in cyclopentyl methyl ether (1.5 mL) and acetonitrile (1.5 mL) containing potassium carbonate (386 mg, 2.8 mmol) and potassium iodide (232 mg, 1.4 mmol) was heated at 85° C. for 48 hours. After cooling to room temperature, the reaction mixture was filtered through Celite, washed with ethyl acetate, and then the solvent was removed under vacuum to give the crude product which was purified by flash chromatography (SiO₂: ethyl acetate/hexane 0-100% with 1% triethylamine in the eluent) to get Compound 19 (93 mg, 18%) as slightly yellow oil. ¹H NMR (300 MHz, CDCl₃): δ ppm 3.54 (m, 2H), 2.78 (t, J=7.7 Hz, 8H), 2.42 (m, 6H), 2.27 (t, J=7.7 Hz, 4H), 1.70-1.16 (m, 68H), 0.86 (t, J=7.1 Hz, 12H); MS (CI): m/z [M+H]⁺ 824; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.1 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=11.6 min, purity: 97.26%.

### Synthesis of Compound 21 (undecyl 6-((8-((dioctylamino)oxy)-8-oxooctyl)(2-hydroxyethyl)amino) hexanoate)

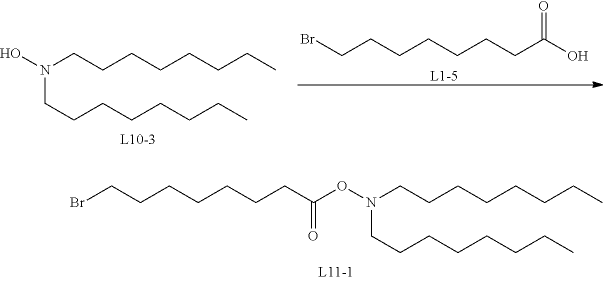

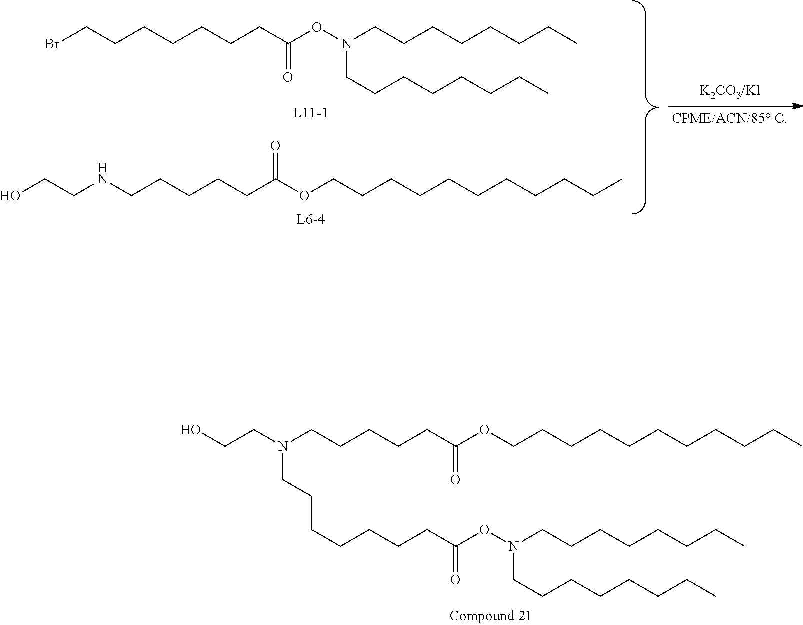 [SMILES: CCCCCCCCN(CCCCCCCC)OC(=O)CCCCCCCBr ; CCCCCCCCCCCOC(=O)CCCCCNCCO ; CCCCCCCCCCCOC(=O)CCCCCN(CCO)CCCCCCCC(=O)ON(CCCCCCCC)CCCCCCCC]

### Synthesis of O-(8-bromooctanoyl)-N,N-dioctylhydroxylamine (L11-1)

To a solution of L10-3 (0.5 g, 1.9 mmol) in DCM (12 mL) was added 7-bromooctanoic acid (0.52 g, 2.3 mmol) followed by DIPEA (0.7 mL, 3.8 mmol), DMAP (0.25 g, 1.9 mmol) and EDC (0.78 g, 3.8 mmol). The resulting mixture was stirred at room temperature under nitrogen atmosphere for 22 h. The reaction mixture was diluted with DCM (10 mL) and washed with saturated NaHCO₃ aqueous solution (10 mL), water (10 mL) and brine (10 mL). The organic phase was dried over anhydrous Na₂SO₄. Filtration and concentration provided crude material which was purified by flash column chromatography (SiO₂: 0 to 100% ethyl acetate in hexane gradient) to yield L11-1 as colorless oil (0.4 g, 38%). ¹H-NMR (300 MHz, CDCl₃) δ 3.39 (t, J=6.5 Hz, 2H), 2.79 (t, J=7.4 Hz, 4H), 2.28 (t, J=7.7 Hz, 2H), 1.90-1.15 (m, 36H), 0.86 (t, J=6.9 Hz, 6H). CIMS m/z [M+H]⁺ 462/464.

### Synthesis of undecyl 6-((8-((dioctylamino)oxy)-8-oxooctyl)(2-hydroxyethyl)amino) hexanoate (Compound 21)

A solution of L11-1 (460 mg, 0.86 mmol), L6-4 (234 mg, 0.72 mmol) in cyclopentyl methyl ether (5 mL) and acetonitrile (5 mL) containing potassium carbonate (300 mg, 2.2 mmol) and potassium iodide (143 mg, 0.86 mmol) was heated at 85° C. for 48 hours. After cooling to room temperature, the reaction mixture was filtered through Celite, washed with ethyl acetate, and then the solvent was removed under vacuum to give the crude product which was purified by flash chromatography (SiO₂: ethyl acetate/hexane 0-100% with 1% triethylamine in the eluent) to get Compound 21 (132 mg, 26%) as slightly yellow oil. ¹H NMR (300 MHz, CDCl₃): δ ppm 4.05 (t, J=6.6 Hz, 2H), 3.53 (m, 2H), 2.79 (t, J=7.1 Hz, 4H), 2.40 (m, 6H), 2.58 (s, bs, 2H), 2.45 (s, bs, 4H), 2.29 (t, J=7.4 Hz, 2H), 2.27 (t, J=7.7 Hz, 2H), 1.70-1.15 (m, 68H), 0.87 (t, J=6.8 Hz, 3H), 0.86 (t, J=6.9 Hz, 6H); MS (CI): m/z [M+H]⁺ 711.6; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.3 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, A in B 60% to 100% in 15 min, flow rate: 0.5 mL/min, column temperature: 20±2° C., detector: CAD, t_{R}=10.2 min, purity: 97.65%.

### Synthesis of Compound 5 (4-(3-methylthioureido)butyl)azanediyl)bis(hexane-6,1-diyl) bis(2-hexyldecanoate)

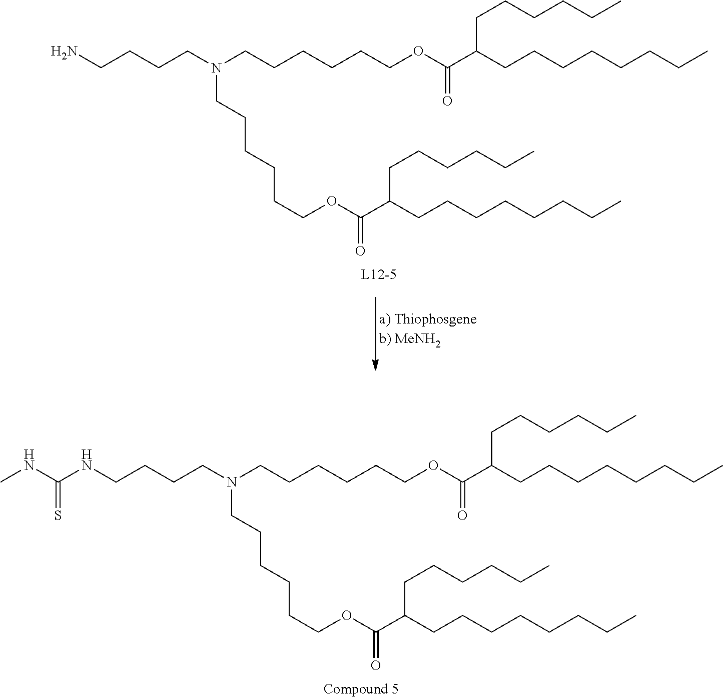

### Synthesis of ((4-(3-methylthioureido)butyl)azanediyl)bis(hexane-6,1-diyl) bis(2-hexyldecanoate) (Compound 5)

To a solution of L12-5 (300 mg, 0.392 mmol) in diethyl ether (30 mL) was added thiophosgene (136 mg, 1.176 mmol) at 0° C. under nitrogen atmosphere. The reaction mixture was then warmed to room temperature and stirred for 3 h. The volatile components were removed under reduced pressure and to the residue was added 2.0 M methyl amine in methanol (1.96 mL, 3.92 mmol). The resulting mixture was stirred at room temperature for 16 h. Solvent was removed under vacuum and the crude was purified by flash column chromatography (SiO₂: 0 to 10% MeOH/DCM with 1% NH₄OH) to yield Compound 5 as sticky gum (158 mg, 48%). ¹H-NMR (300 MHz, CDCl₃) δ. 4.05 (t, 4H), 3.65 (b, 2H), 3.04 (d, 3H), 2.90 (b, 4H), 2.34-2.24 (m, 2H), 1.98-1.48 (m, 20H), 1.48-1.15 (m, 52H), 0.87 (t, 12H); CIMS m/z [M+H]⁺ 838.8. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.27 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.83 min, purity: 92.71%.

### Synthesis of Compound 61 ((2-((2-hydroxyethyl) (methyl)amino)ethyl)azanediyl)bis(hexane-6,1-diyl) bis(4,4-bis(octyloxy)butanoate)

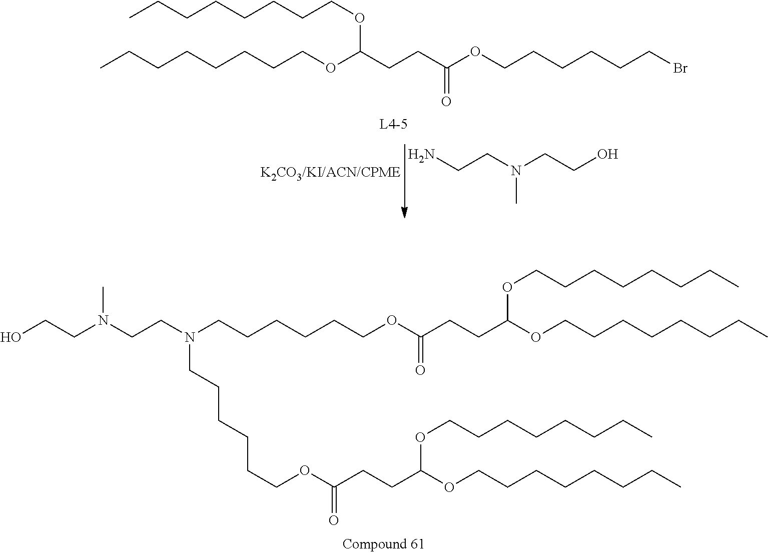

### Synthesis of ((2-((2-hydroxyethyl) (methyl)amino)ethyl)azanediyl)bis(hexane-6,1-diyl) bis(4,4-bis(octyloxy)butanoate) (Compound 61)

To a solution of 6-bromohexyl 4,4-bis(octyloxy)butanoate L4-5 (2.13 g, 4.2 mmol) in acetonitrile (10 mL) and cyclopentylmethylether (10 mL) under nitrogen was added 2-((2-aminoethyl)-(methyl)amino)ethan-1-ol (224 mg, 1.9 mmol), K₂CO₃ (1.57 g, 11.4 mmol) and KI (315 mg, 1.9 mmol). The reaction mixture was heated at 80° C. overnight. After cooling to room temperature, the reaction mixture was filtered through Celite, and the filter cake was washed with ethyl acetate. The combined filtrates were concentrated to give crude product, which was purified by flash chromatography (SiO₂: 0 to 5% MeOH in DCM gradient) to yield Compound 61 as colorless oil (652 mg, 35%). ¹H-NMR (300 MHz, CDCl₃) δ 4.48 (t, J=5.7, 2H), 4.04 (t, J=6.5, 4H), 3.56 (m, 6H), 3.38 (m, 4H), 2.6-2.8 (m, 6H), 2.37 (m, 7H), 1.9 (m, 4H), 1.52-1.6 (m, 16H), 1.26-1.35 (m, 52H), 0.87 (m, 12H); CIMS m/z [M+H]⁺ 971.8; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.45 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=11.19 min, purity: 98.46%.

### Synthetic Scheme for Compound 77

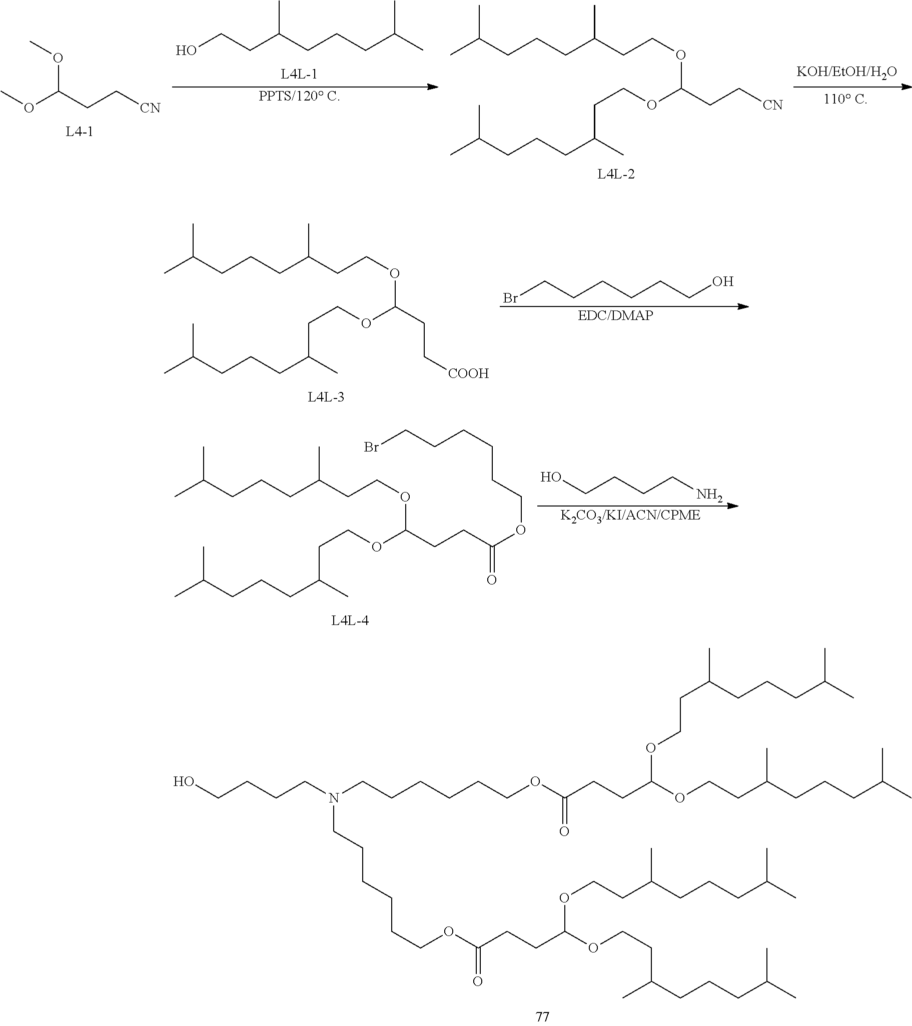

### Synthesis of 4,4-bis(3,7-dimethyloctyl)oxy)butane nitrile (L4L-2) [Procedure A]

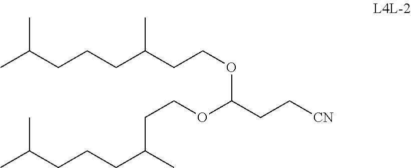 [SMILES: CC(C)CCCC(C)CCOC(CCC#N)OCCC(C)CCCC(C)C]

To a 100 mL round bottom flask, 4,4-dimethoxybutanenitrile (3.0 g, 23.2 mmol), alcohol (11.0 g, 69.7 mmol) and pyridinium p-toluenesulfonate (0.29 g 1.2 mmol) were added. The resulting mixture was stirred at 120° C. for 4 h and cooled to room temperature. EtOAc (50 mL) and H₂O (20 mL) were added in, and the resulting phases were separated. The aqueous phase was extracted with EtOAc (50 mL). Combined organic extracts were washed with H₂O (20 mL) and dried over anhydrous MgSO₄. Filtration and concentration provided crude material which was purified by flash column chromatography (SiO₂: 0 to 10% ethyl acetate in hexanes gradient) to yield L4L-2 as colorless oil (6.6 g, 74%); ¹HNMR (CDCl₃) δ 4.50-4.53 (t, 1H), 3.58-3.60 (m, 2H), 3.41-3.49 (m, 2H), 2.39-2.44 (t, 2H), 1.92-1.94 (q, 2H), 1.50-1.55 (m, 6H), 1.38-1.42 (m, 2H), 1.11-1.14 (m, 14H) 0.88-0.84 (t, 18H); CIMS m/z [M+H]⁺ 381.

### Synthesis of 4,4-bis((3,7-dimethyloctyl) oxy) butanoic acid (L4L-3) [Procedure B]

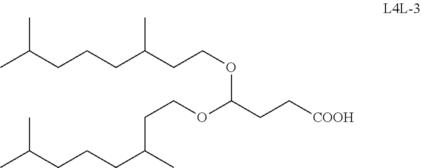 [SMILES: CC(C)CCCC(C)CCOC(CCC(=O)O)OCCC(C)CCCC(C)C]

To a 100 mL round bottom flask containing a solution of L4L-2 (8.2 g, 21 mmol) in ethanol (50 mL) was added a solution of KOH (3.6 g, 64 mmol) in water (50 mL). The mixture was stirred at 120° C. for 20 h. The volatiles were removed, and the reaction pH was adjusted to 5. EtOAc (150 mL) and H₂O (60 mL) were added, and the resulting phases were separated. The aqueous phase was extracted with EtOAc (50 mL). Combined organic extracts were washed with H₂O (60 mL×2) and dried over anhydrous MgSO₄. Filtration and concentration provided L4L-3 (6.4 g, 74%) which was used for the next step without further purification. ¹HNMR (CDCl₃) δ 4.54 (t, 1H), 3.60-3.65 (m, 2H), 3.45-3.49 (m, 2H), 2.39-2.44 (t, 2H), 1.92-1.94 (m, 2H), 1.50-1.95 (m, 6H), 1.26-1.55 (m, 8H), 1.11-1.14 (m, 6H). 0.84-0.88 (d, 18H); CIMS m/z [M−H]⁻ 399.

### Synthesis of 6-bromohexyl 4,4-bis((3,7-dimethyloctyl)oxy)butanoate (L4L-4) [Procedure C]

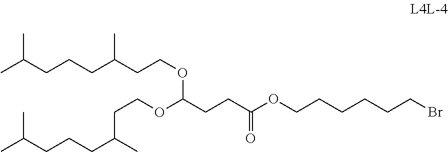 [SMILES: CC(C)CCCC(C)CCOC(CCC(=O)OCCCCCCBr)OCCC(C)CCCC(C)C]

To an oven dried 100 mL round bottom flask containing a solution of acid L4L-3 (1.5 g, 3.8 mmol) in dichloromethane (10 mL) under nitrogen was added EDC (1.44 g, 7.6 mmol), DMAP (92 mg, 0.76 mmol). The mixture was stirred at room temperature for 15 min. The alcohol (0.61 g, 3.38 mmol) was then added and the reaction mixture was stirred for 20 h at room temperature. The reaction mixture was diluted with dichloromethane and water and the resulting phases were separated. The aqueous phase was extracted again with EtOAc (50 mL). Combined organic extracts were washed with H₂O (60 mL×2) and dried over anhydrous MgSO₄. Filtration and concentration provided crude product which was purified by flash column chromatography (SiO₂: 0 to 10% ethyl acetate in hexanes gradient) to yield L4L-4 as colorless oil (1.1 g, 58%); ¹H-NMR (300 MHz, CDCl₃) δ 4.49 (t, J=5.4 Hz, 1H), 4.05 (t, J=6.7 Hz, 2H), 3.70-3.33 (m, 6H), 2.36 (t, J=7.7 Hz, 2H), 1.99-1.77 (m, 2H), 1.70-1.00 (m, 28H), 0.87 (t, J=6.6 Hz, 18H).

### Synthesis of ((4-hydroxybutyl)azanediyl)bis(hexane-6,1-diyl) bis(4,4-bis((3,7-dimethyloctyl)oxy)butanoate) (77) [Procedure D]

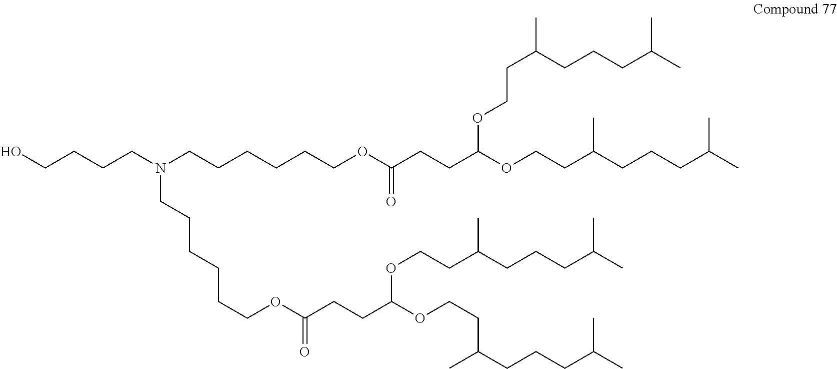 [SMILES: CC(C)CCCC(C)CCOC(CCC(=O)OCCCCCCN(CCCCO)CCCCCCOC(=O)CCC(OCCC(C)CCCC(C)C)OCCC(C)CCCC(C)C)OCCC(C)CCCC(C)C]

To a solution of compound L4L-4 (1.1 g, 1.9 mmol) in CH₃CN/CPME (1:1, 10 mL) under nitrogen was added 4-amino-1-butanol (79 mg, 0.88 mmol), followed by the addition of K₂CO₃ (0.37 g, 2.6 mmol) and KI (0.15 g, 0.88 mmol). The reaction mixture was heated at 80-90° C. for 18 h. After cooling to room temperature, the reaction mixture was filtered through Celite, washed with ethyl acetate, and the solvent was removed under vacuum to give the crude product which was purified by flash chromatography (SiO₂: DCM/MeOH 0-10%) to get final Compound 77 as slightly yellow oil (619 mg, 68%); ¹H-NMR (300 MHz, CDCl₃) δ 4.48 (t, J=5.4 Hz, 2H), 4.04 (t, J=6.8 Hz, 4H), 3.70-3.35 (m, 10H), 2.51-2.32 (m, IOH), 1.96-1.87 (m, 4H), 1.72-1.03 (m, 60H), 0.91-0.83 (m, 36H); MS (CI): m/z [M+H]⁺ 1054.9; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.8 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=15.4 min, purity: >99%.

### Synthetic Scheme for Compound 78

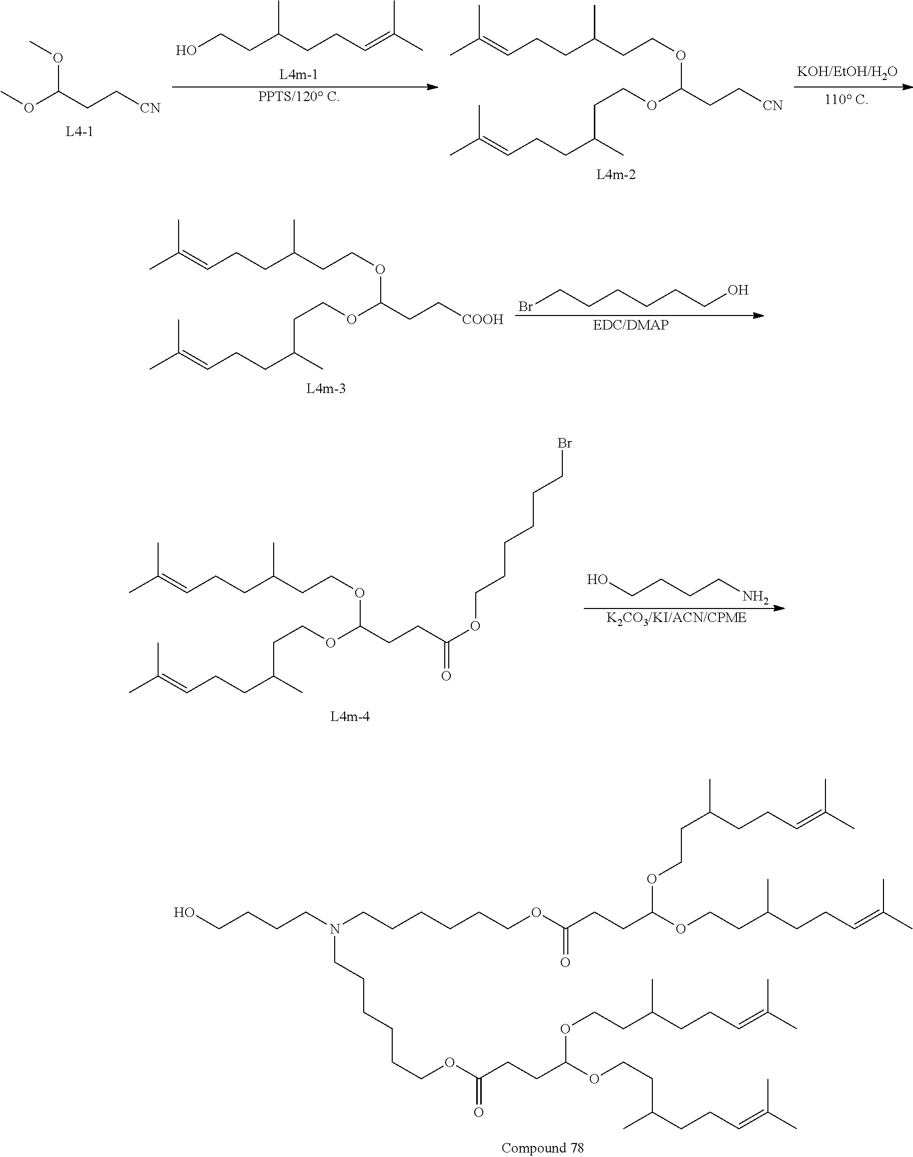

### Synthesis of 4,4-bis((3,7-dimethyloct-6-en-1-yl)oxy)butanenitrile (L4m-2)

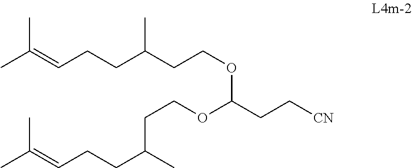 [SMILES: CC(C)=CCCC(C)CCOC(CCC#N)OCCC(C)CCC=C(C)C]

Prepared following Procedure A described in Compound 77 synthesis. Compound L4m-2 was isolated as colorless oil (6.2 g, 73%). ¹H-NMR (300 MHz, CDCl₃) δ 5.08 (t, J=7.2 Hz, 2H), 4.54 (t, J=5.3 Hz, 1H), 3.72-3.36 (m, 4H), 2.41 (t, J=7.3 Hz, 2H), 2.10-1.84 (m, 6H), 1.80-1.05 (m, 22H), 0.89 (d, J=6.6 Hz, 6H).

### Synthesis of 4,4-bis((3,7-dimethyloct-6-en-1-yl)oxy)butanoic acid (L4m-3)

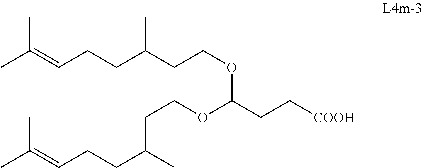 [SMILES: CC(C)=CCCC(C)CCOC(CCC(=O)O)OCCC(C)CCC=C(C)C]

Prepared following Procedure B described in Compound 77 synthesis. Compound L4m-3 was isolated as colorless oil (6.2 g, 73%). ¹H-NMR (300 MHz, CDCl₃) δ: 5.08 (t, J=6.84 Hz, 2H), 4.5 (t, J=5.22 Hz, 11H), 3.59 (m, 2H), 3.44 (m, 2H), 2.44 (t, J=7.44 Hz, 2H), 1.94 (m, 6H), 1.54-1.67 (m, 16H), 1.35 (m, 4H), 1.13 (m, 2H), 0.87 (d, J=6.33 Hz, 6H); CIMS m/z [M+H]⁺ 396.2.

### Synthesis of 6-bromohexyl 4,4-bis((3,7-dimethyloct-6-en-1-yl)oxy)butanoate (L4m-4)

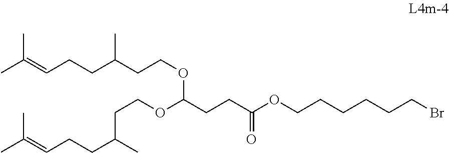 [SMILES: CC(C)=CCCC(C)CCOC(CCC(=O)OCCCCCCBr)OCCC(C)CCC=C(C)C]

Prepared following Procedure C described in Compound 77 synthesis. Compound L4m-4 was isolated as colorless oil (880 mg, 88%). ¹H-NMR (300 MHz, CDCl₃) δ: 5.08 (t, J=7.14 Hz, 2H), 4.48 (t, J=5.49 Hz, 1H), 4.06 (t, J=6.57 Hz, 2H), 3.57 (m, 2H), 3.43 (m, 2H), 3.4 (t, J=6.87 Hz, 2H), 2.37 (t, J=7.41 Hz, 2H) 1.93 (m, 8H), 1.55-1.67 (m, 16H), 1.36 (m, 10H), 1.15 (m, 2H), 0.87 (d, J=6.33 Hz, 6H).

### Synthesis of Compound 78 ((4-hydroxybutyl)azanediyl)bis(hexane-6,1-diyl) bis(4,4-bis((3,7-dimethyloct-6-en-1-yl)oxy)butanoate)

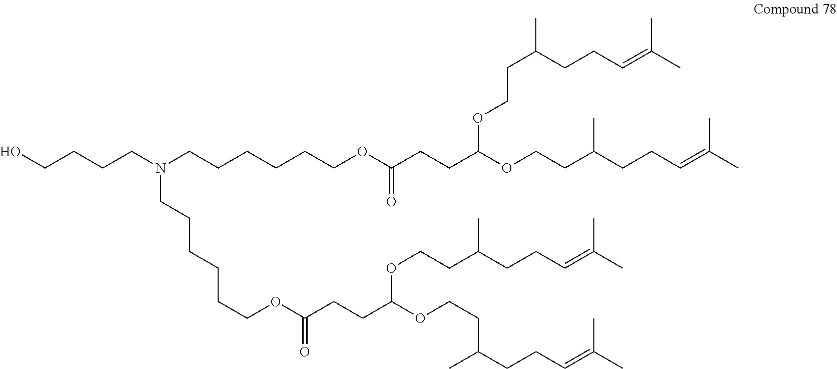 [SMILES: CC(C)=CCCC(C)CCOC(CCC(=O)OCCCCCCN(CCCCO)CCCCCCOC(=O)CCC(OCCC(C)CCC=C(C)C)OCCC(C)CCC=C(C)C)OCCC(C)CCC=C(C)C]

Prepared following Procedure D described in Compound 77 synthesis. Compound 78 was isolated as colorless oil (415 mg, 53%). ¹H-NMR (300 MHz, CDCl₃) δ: 5.08 (t, J=7.14 Hz, 4H), 4.46 (t, J=5.4 Hz, 2H), 4.04 (t, J=6.5 Hz, 4H), 3.54-3.6 (m, 4H), 3.41-3.44 (m, 4H), 2.37 (m, 6H), 1.95 (m, 12H), 1.56-1.67 (m, 50H), 1.35 (m, 16H), 1.16 (m, 4H), 0.87 (m, 12H); CIMS m/z [M+H]⁺ 1046.6. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.4 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=14.1 min, purity: 96.7%.

### Synthetic Scheme for Compound 79

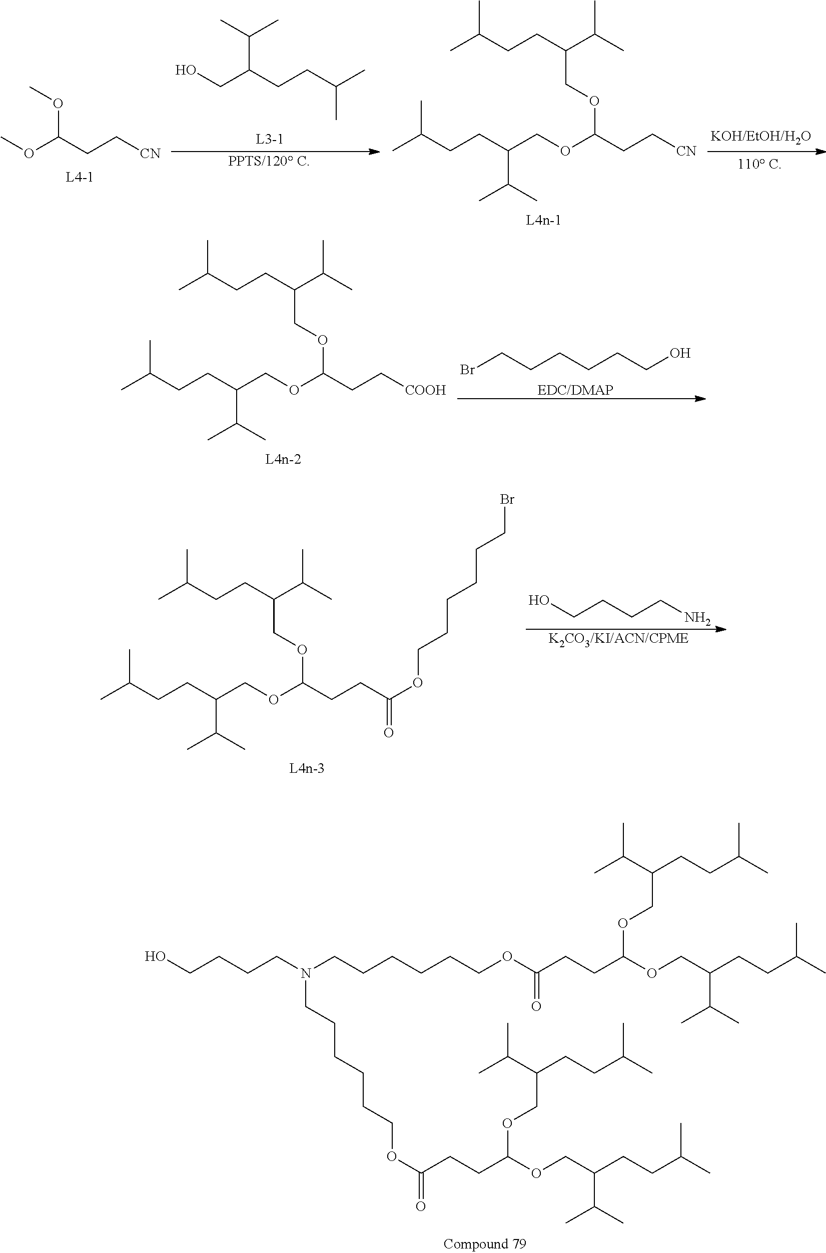

### Synthesis of 4,4-bis((2-isopropyl-5-methylhexyl)oxy)butanenitrile (L4n-1)

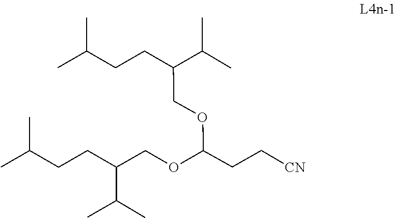 [SMILES: CC(C)CCC(COC(CCC#N)OCC(CCC(C)C)C(C)C)C(C)C]

Prepared following Procedure A described in Compound 77 synthesis. Compound (L4n-1) was isolated as light-yellow oil in a yield of 3.66 g (92%). ¹H-NMR (300 MHz, CDCl₃) δ 4.48 (dd, 1H), 3.62-3.49 (m, 2H), 3.42-3.31 (m, 2H), 2.42 (t, 2H), 1.97-1.78 (m, 2H), 1.58-1.42 (m, 2H), 1.41-1.08 (m, 10H), 0.87 (m, 24H).

### Synthesis of 4,4-bis((2-isopropyl-5-methylhexyl)oxy)butanoic acid (L4n-2)

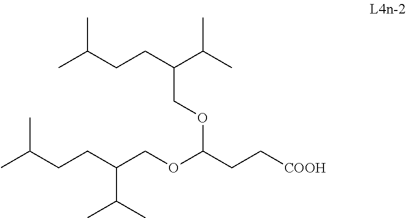 [SMILES: CC(C)CCC(COC(CCC(=O)O)OCC(CCC(C)C)C(C)C)C(C)C]

Prepared following Procedure B described in Compound 77 synthesis. Compound (L4n-2) was isolated as light-yellow oil in a yield of 3.7 g (98%). ¹H-NMR (300 MHz, CDCl₃) δ 4.48 (dd, 1H), 3.62-3.49 (m, 2H), 3.42-3.31 (m, 4H), 2.38 (t, 2H), 1.94 (q, 2H), 1.83-1.67 (m, 2H), 1.54-1.39 (m, 2H), 1.38-1.01 (m, 10H), 0.87 (m, 24H).

### Synthesis of 6-bromohexyl 4,4-bis((2-isopropyl-5-methylhexyl)oxy)butanoate (L4n-3)

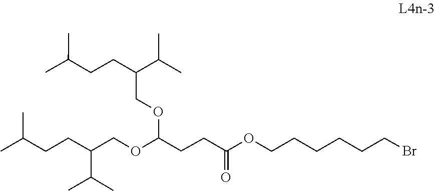 [SMILES: CC(C)CCC(COC(CCC(=O)OCCCCCCBr)OCC(CCC(C)C)C(C)C)C(C)C]

Prepared following Procedure C described in Compound 77 synthesis. Compound (L4n-3) was isolated as light-yellow oil in a yield of 1.8 g (85%). ¹H-NMR (300 MHz, CDCl₃) δ 4.44 (dd, 1H), 4.06 (t, 2H), 3.55-3.43 (m, 2H), 3.40 (t, 2H), 3.31-3.18 (m, 2H), 2.38 (t, 2H), 1.94-1.71 (m, 6H), 1.68-1.54 (m, 2H), 1.54-1.01 (m, 16H), 0.87 (m, 24H).

### Synthesis of ((4-hydroxybutyl)azanediyl)bis(hexane-6,1-diyl) bis(4,4-bis((2-isopropyl-5-methylhexyl)oxy)butanoate) (Compound 79)

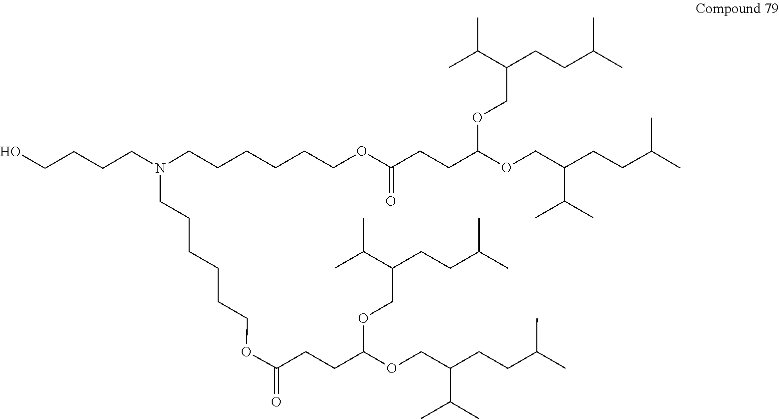 [SMILES: CC(C)CCC(COC(CCC(=O)OCCCCCCN(CCCCO)CCCCCCOC(=O)CCC(OCC(CCC(C)C)C(C)C)OCC(CCC(C)C)C(C)C)OCC(CCC(C)C)C(C)C)C(C)C]

Prepared following Procedure D described in Compound 77 synthesis. Compound 79 was isolated as light yellow oil in a yield of 1.21 g (81%). ¹H-NMR (300 MHz, CDCl₃) δ 4.43 (dd, 2H), 4.05 (t, 4H), 3.66-3.56 (m, 2H), 3.55-3.44 (m, 4H), 3.35-3.23 (m, 4H), 2.90-2.46 (bs, 6H), 2.37 (t, 4H), 1.92 (q, 4H), 1.86-1.72 (m, 6H), 1.70-1.53 (m, 12H), 1.54-1.43 (m, 6H), 1.42-1.24 (m, 16H), 1.24-1.04 (m, 14H), 0.87 (m, 48H); CIMS m/z [M+H]⁺ 1055.1. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.61 min, purity: >99.9%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=7.67 min, purity: 98.2%.

### Synthetic Scheme for Compound 84

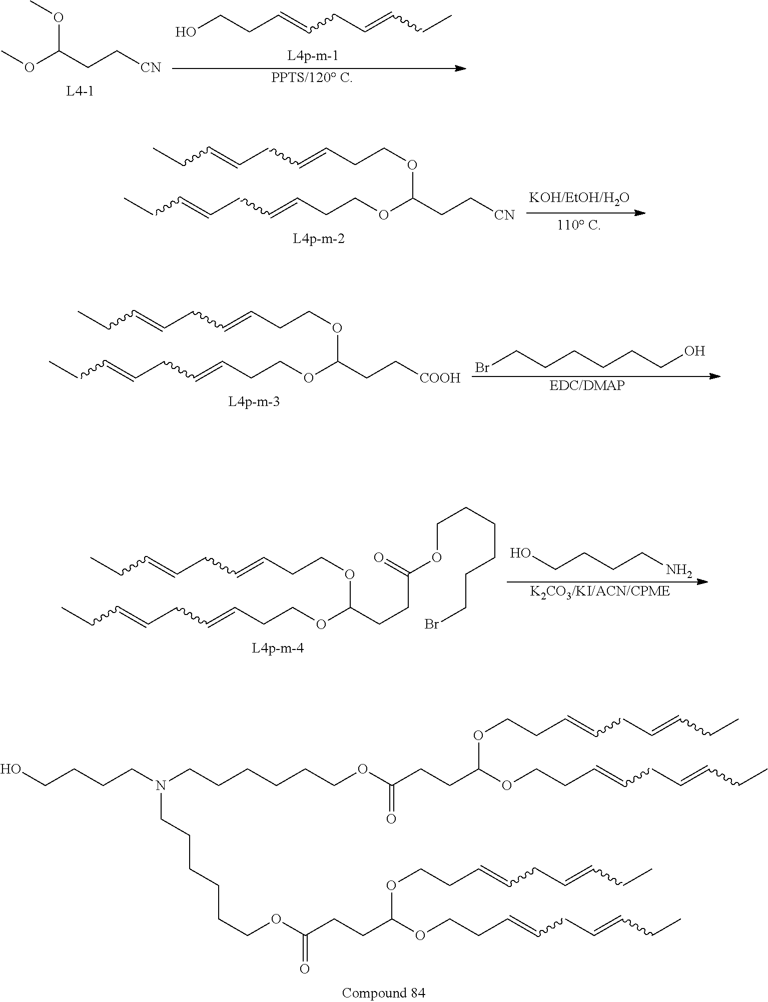

### Synthesis of 4,4-bis(nona-3,6-dien-1-yloxy)butanenitrile L4p-m-2

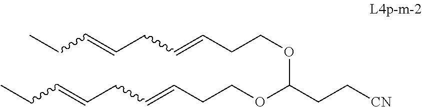 [SMILES: CCC=CCC=CCCOC(CCC#N)OCCC=CCC=CCC]

Prepared following Procedure A described in Compound 77 synthesis. L4p-m-2 was isolated as colorless oil (1.45 g, 54%). ¹H-NMR (300 MHz, CDCl₃) δ 5.47-5.33 (m, 8H), 4.56 (t, 1H), 3.63-3.42 (m, 4H), 2.73 (t, 4H), 2.40 (t, 2H), 2.28-2.24 (m, 4H), 2.06-1.90 (m, 6H), 0.95 (t, 6H).

### Synthesis of 4,4-bis(nona-3,6-dien-1-yloxy)butanoic acid L4p-m-3

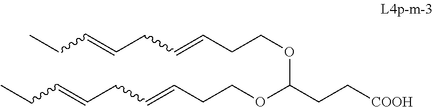 [SMILES: CCC=CCC=CCCOC(CCC(=O)O)OCCC=CCC=CCC]

Prepared following Procedure B described in Compound 77 synthesis. Compound L4p-m-3 (1.35 g, 88%); ¹H-NMR (300 MHz, CDCl₃) δ 5.46-5.28 (m, 8H), 4.53 (t, 1H), 3.61-3.41 (m, 4H), 2.73 (t, 4H), 2.44 (t, 2H), 2.29-2.23 (m, 4H), 2.06-1.90 (m, 6H), 0.95 (t, 6H).

### Synthesis of 6-bromohexyl 4,4-bis(nona-3,6-dien-1-yloxy)butanoate L4p-m-4

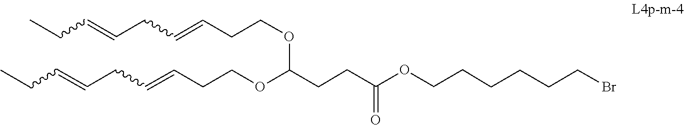 [SMILES: CCC=CCC=CCCOC(CCC(=O)OCCCCCCBr)OCCC=CCC=CCC]

Prepared following Procedure C described in Compound 77 synthesis. Compound L4p-m-4 was isolated as colorless oil (1.08 g, 51%).

### Synthesis of ((4-hydroxybutyl)azanediyl)bis(hexane-6,1-diyl) bis(4,4-bis(nona-3,6-dien-1-yloxy)butanoate) (Compound 84)

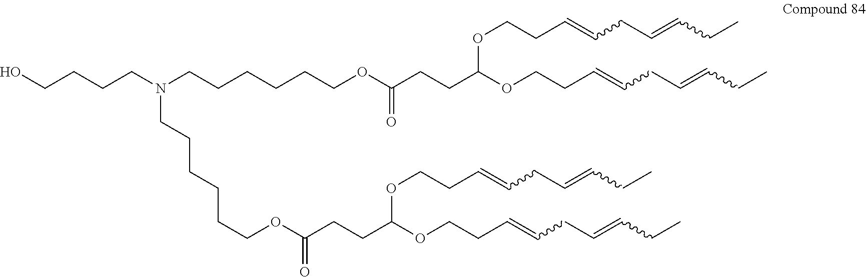 [SMILES: CCC=CCC=CCCOC(CCC(=O)OCCCCCCN(CCCCO)CCCCCCOC(=O)CCC(OCCC=CCC=CCC)OCCC=CCC=CCC)OCCC=CCC=CCC]

Prepared following Procedure D described in Compound 77 synthesis. Compound 84 was isolated as colorless oil (0.610 g, 61%); ¹H-NMR (300 MHz, CDCl₃) δ 5.51-5.29 (m, 16H), 4.51 (t, 2H), 4.04 (t, 4H), 3.62-3.37 (m, 10H), 2.73 (t, 8H), 2.55 (bs, 5H), 2.36 (t, 4H), 2.25 (q, 8H), 2.08-1.88 (m, 12H), 1.74-1.18 (m, 28H), 0.95 (t, 12H); CIMS m/z [M+H]⁺ 982.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=7.44 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=11.39 min, purity: 98.3%.

### Synthetic Scheme for Compound 81

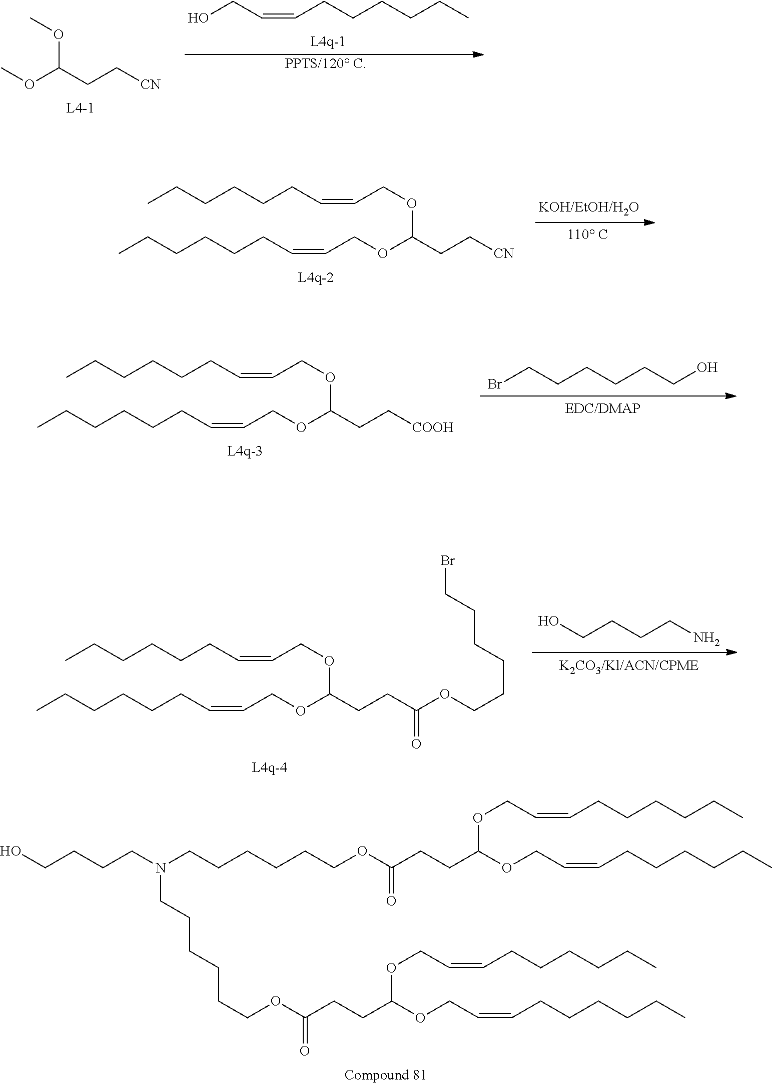

### Synthesis of 4,4-bis(((Z)-non-2-en-1-yl)oxy)butanenitrile (L4q-2)

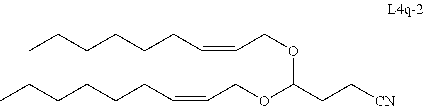 [SMILES: CCCCCC/C=C\COC(CCC#N)OC/C=C\CCCCCC]

Prepared following Procedure A described in Compound 77 synthesis. Compound L4q-2 as pale Brown oil (2.0 g, 74%); ¹H-NMR (300 MHz, CDCl₃) δ 5.58-5.47 (m, 4H), 4.65 (t, 1H), 4.19-4.06 (m, 4H), 2.43 (t, 2H), 2.07-1.92 (m, 6H), 1.37-1.18 (m, 16H), 0.88 (t, 6H).

### Synthesis of 4,4-bis(((Z)-non-2-en-1-yl)oxy)butanoic acid (L4q-3)

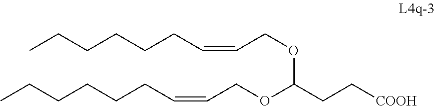 [SMILES: CCCCCC/C=C\COC(CCC(=O)O)OC/C=C\CCCCCC]

Prepared following Procedure B described in Compound 77 synthesis. Compound L4q-3 was isolated as light brown oil (1.93 g, 92%); ¹H-NMR (300 MHz, CDCl₃) δ 5.60-5.47 (m, 4H), 4.62 (t, 1H), 4.18-4.04 (m, 4H), 2.48-2.43 (t, 2H), 2.08-1.93 (m, 6H), 1.40-1.18 (m, 16H), 0.87 (t, 6H).

### Synthesis of 6-bromohexyl 4,4-bis(((Z)-non-2-en-1-yl)oxy)butanoate (L4q-4)

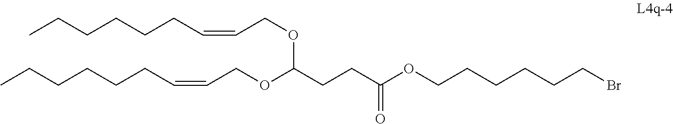 [SMILES: CCCCCC/C=C\COC(CCC(=O)OCCCCCCBr)OC/C=C\CCCCCC]

Prepared following Procedure C described in Compound 77 synthesis. Compound L4q-4 was isolated as light brown oil (1.08 g, 37%); ¹H-NMR (300 MHz, CDCl₃) δ 5.58-5.46 (m, 4H), 4.59 (t, 1H), 4.13-4.02 (m, 6H), 3.40 (t, 2H), 2.39 (t, 2H), 2.08-1.81 (m, 8H), 1.68-1.58 (m, 2H), 1.50-1.18 (m, 20H), 0.87 (t, 6H).

### Synthesis of ((4-hydroxybutyl)azanediyl)bis(hexane-6,1-diyl) bis(4,4-bis(((Z)-non-2-en-1-yl)oxy)butanoate) (Compound 81)

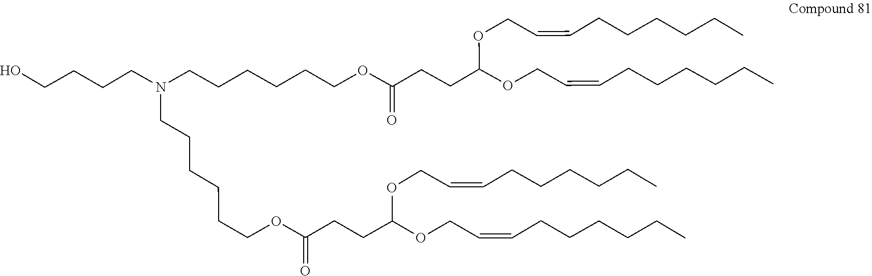 [SMILES: CCCCCC/C=C\COC(CCC(=O)OCCCCCCN(CCCCO)CCCCCCOC(=O)CCC(OC/C=C\CCCCCC)OC/C=C\CCCCCC)OC/C=C\CCCCCC]

Prepared following Procedure D described in Compound 77 synthesis. Compound 81 was isolated as light brown oil (0.62 g, 62%). ¹H-NMR (300 MHz, CDCl₃) δ 5.58-5.44 (m, 8H), 4.59 (t, 2H), 4.15-4.0 (m, 13H), 3.60 (bs, 2H), 2.70-2.48 (m, 4H), 2.39 (t, 4H), 2.08-1.92 (m, 12H), 1.78-1.44 (m, 14H), 1.42-1.18 (m, 43H), 0.87 (t, 12H); CIMS m/z [M+H]⁺ 990.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.19 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.94 min, purity: 94.66%.

### Synthetic Scheme for Compound 82:

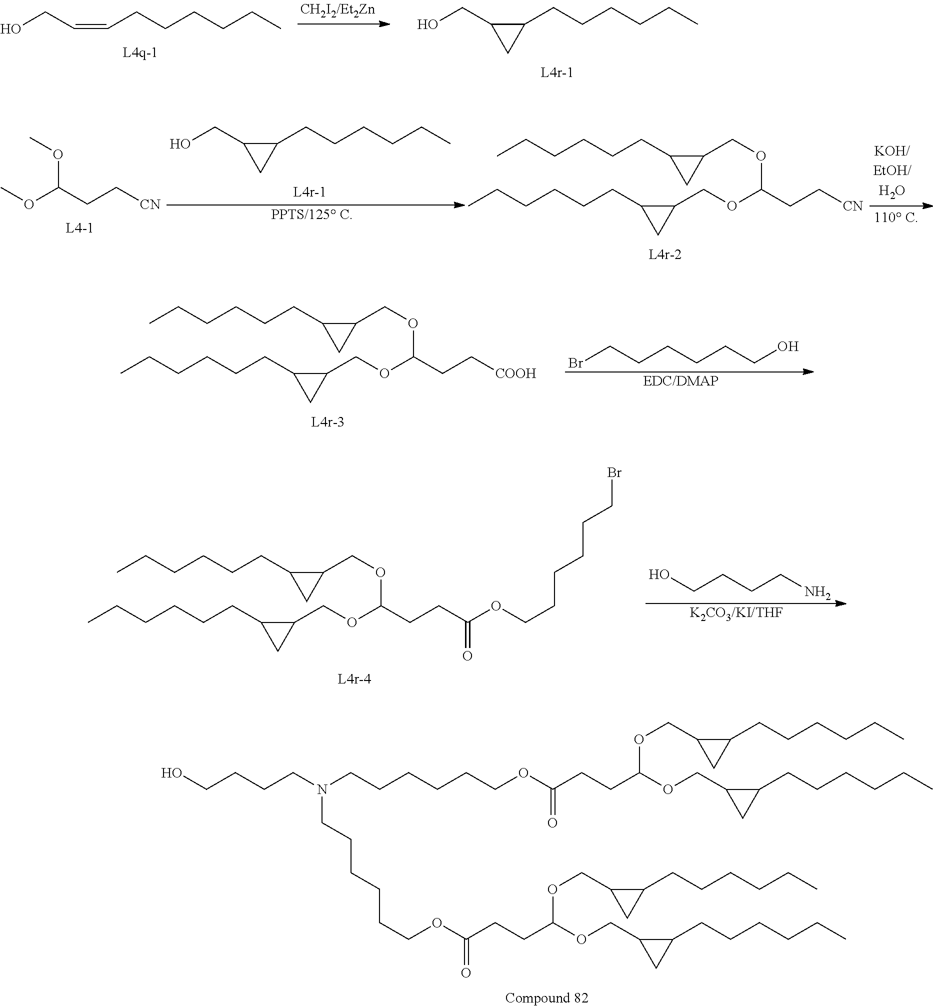

### Synthesis of (2-hexylcyclopropyl)methanol (L4r-1)

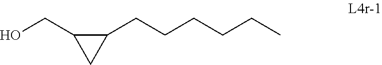

To an oven dry 500 mL three neck round bottom flask equipped with a 100 mL dropping funnel, a solution of diethyl zinc (IM in hexane, 50 mL) was added along with anhydrous dichloromethane (50 mL) and the contents were stirred at −40° C. under nitrogen atmosphere for 30 min. Diiodomethane (8.05 mL, 100 mmol) dissolved in anhydrous dichloromethane (30 mL) was added dropwise to the reaction. The resulting mixture was stirred at the same temperature for 60 min. To this mixture was added dropwise a solution of trichloroacetic acid (817.5 mg, 5 mmol) in anhydrous dichloromethane (20 mL) and anhydrous dimethoxy ethane (3 mL) and the reaction mixture was stirred for 1 h at −40° C. then allowed to warm to −15° C. in 1 h. A solution of L4q-1 in anhydrous dichloromethane (30 mL) was added dropwise to the reaction and the contents were stirred at −15° C. for 1 h then allowed to warm to room temperature and stirred overnight. After the completion of the reaction, the contents were added to a 2000 mL conical flask containing saturated NH₄Cl (300 mL) and stirred for 15 min. The crude product was extracted with dichloromethane (100 mL×3). Combined organic phases were washed with brine and dried over anhydrous magnesium sulphate. The solvent was removed under vacuum to give crude product (4.0 g). This crude product was subjected to next step reaction without further purification.)H NMR (300 MHz, CDCl₃): δ ppm 3.68-3.54 (m, 2H), 1.58-1.20 (m, 15H), 1.18-0.85 (m, 4H), 0.85-0.68 (m, 1H), 0.0-−0.04 (m, 1H); CIMS m/z [M+H]⁺ 157.32.

### Synthesis of 4,4-bis((2-hexylcyclopropyl)methoxy)butanenitrile (Compound L4r-2)

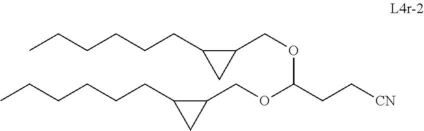 [SMILES: CCCCCCC1CC1COC(CCC#N)OCC1CC1CCCCCC]

Prepared following Procedure A described in Compound 77 synthesis. Compound L4r-2 (2.55 g, 87%); ¹H NMR (300 MHz, CDCl₃): δ 4.76-4.63 (m, 1H), 3.76-3.54 (m, 1H), 3.53-3.51 (m, 2H), 3.51 (t, J=6.0 Hz, 1H), 2.45 (t, J=6.0 Hz, 2H), 2.00 (t, J=6.0 Hz, 2H), 1.50-0.92 (m, 22H), 0.89-0.85 (m, 8H), 0.74-0.71 (m, 2H), 0.00-−0.5 (m, 2H); CIMS m/z [M+H]⁺ 378.71.

### Synthesis of 4,4-bis((2-hexylcyclopropyl)methoxy)butanoic acid (L4r-3)

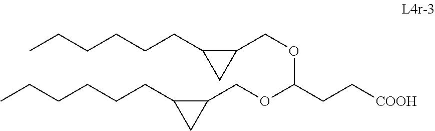 [SMILES: CCCCCCC1CC1COC(CCC(=O)O)OCC1CC1CCCCCC]

Prepared following Procedure B described in Compound 77 synthesis. Compound L4r-3 (2.0 g, 76%) was isolated as colorless oil. ¹H NMR (300 MHz, CDCl₃): δ 4.68-4.57 (m, 1H), 3.68-3.59 (m, 1H), 3.58-3.49 (m, 2H), 3.39 (t, J=7.0 Hz, 1H), 2.49 (t, J=7.0 Hz, 2H), 1.98 (t, J=7.0 Hz, 2H), 1.53-0.85 (m, 30H), 0.72-0.70 (m, 2H), −0.009-−0.06 (m, 2H); CIMS m/z [M+H]⁺ 397.61.

### Synthesis of 6-bromohexyl 4,4-bis((2-hexylcyclopropyl)methoxy)butanoate (L4r-4)

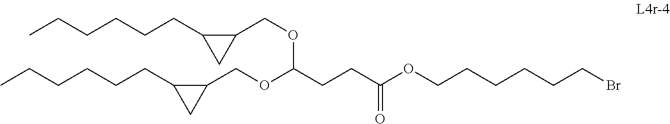 [SMILES: CCCCCCC1CC1COC(CCC(=O)OCCCCCCBr)OCC1CC1CCCCCC]

Prepared following Procedure C described in Compound 77 synthesis. Compound L4r-4 (1.33 g, 48%) as slightly yellow oil. ¹H NMR (300 MHz, CDCl₃): (4.68-4.57 (m, 1H), 4.22-4.16 (m, 1H), 4.09 (t, J=6.0 Hz, 1H), 3.98-3.91 (m, 1H), 3.51-3.49 (m, 2H), 3.51-3.48 (m, 1H), 2.49 (t, J=7.0 Hz, 2H), 1.98 (t, J=7.0 Hz, 2H), 1.53-0.85 (m, 30H), 0.90-0.85 (m, 10H), 0.72-0.69 (m, 2H), −0.008-−0.073 (m, 2H); CIMS m/z [M+H]⁺ 560.68.

### Synthesis of ((4-hydroxybutyl)azanediyl)bis(hexane-6,1-diyl)bis(4,4-bis((2-hexylcyclopropyl)methoxy)butanoate) (82)

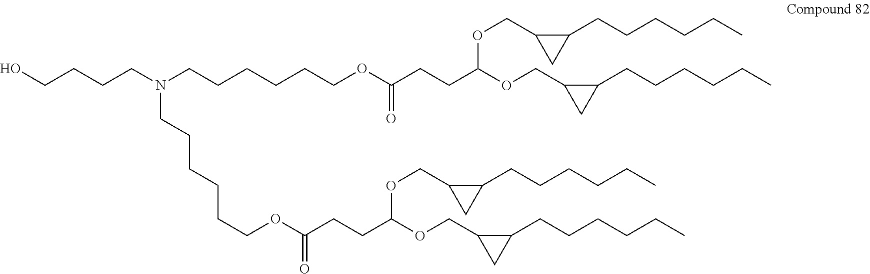 [SMILES: CCCCCCC1CC1COC(CCC(=O)OCCCCCCN(CCCCO)CCCCCCOC(=O)CCC(OCC1CC1CCCCCC)OCC1CC1CCCCCC)OCC1CC1CCCCCC]

Prepared following Procedure D described in Compound 77 synthesis. Compound 82 (810 mg, 77%). ¹H NMR (300 MHz, CDCl₃): δ 4.78-4.67 (m, 2H), 4.05 (t, J=6.0 Hz, 4H), 3.78-3.35 (m, 10H), 2.51-2.40 (m, 10H), 1.99-1.90 (m, 4H), 1.73-1.05 (m, 66H), 0.99-0.75 (m, 19H), −0.007-−0.071 (m, 4H); CIMS m/z [M+H]⁺ 1047.77. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >99%; UPLC column: Waters Aquity UPLC CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=14.8 min, purity: >99%.

### Synthetic Scheme for Compound 129:

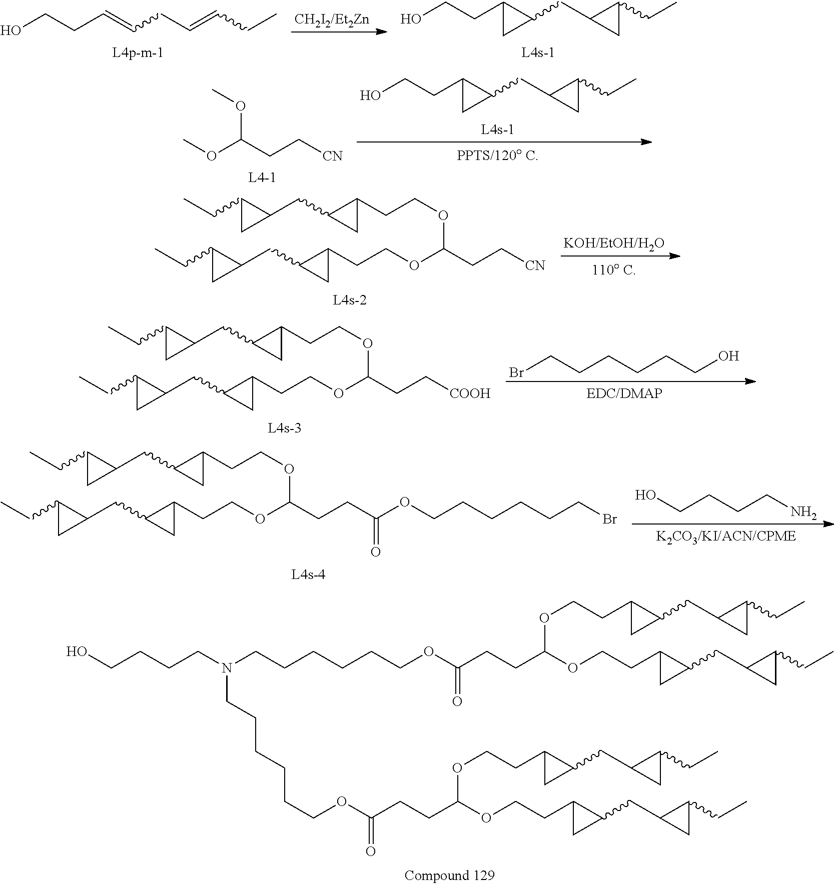

### Synthesis of 2-(2-((2-ethylcyclopropyl)methyl)cyclopropyl)ethan-1-ol (L4s-1)

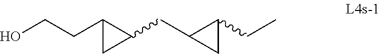

To an oven dry three neck 250 mL round bottom flask equipped with a 50 mL dropping funnel, a solution of diethyl zinc (0.9M in hexane, 72 mL) was added along with anhydrous dichloromethane (30 mL) and the contents were stirred at −40° C. under nitrogen atmosphere for 30 min. Diiodomethane (8.1 mL, 98.84 mmol) dissolved in anhydrous dichloromethane (20 mL) was added dropwise to the reaction. The resulting mixture was stirred at −40° C. for 60 min. To this mixture was added dropwise a solution of trichloroacetic acid (2.56 g, 15.68 mmol) in anhydrous dichloromethane (20 ml) and anhydrous dimethoxy ethane (6.7 mL) and the reaction mixture was stirred for 1 h at −40° C. then allowed to warm to −15° C. in 1 h. A solution of L4p-m-1 (2.2 g, 15.68 mmol) in anhydrous dichloromethane (10 mL) was added dropwise to the reaction and the contents were stirred at −15° C. for 1 h then allowed to warm to room temperature and stirred overnight. After the completion of the reaction, the contents were added to a 1000 mL conical flask containing saturated NH₄Cl (400 mL) and stirred for 15 min. The crude product was extracted using dichloromethane (200 mL×3) from this mixture. Combined organic phases were washed with brine and dried over anhydrous magnesium sulphate. The solvent was removed under vacuum to give crude product. The crude material was adsorbed on 30 g flash silica and loaded on to an empty flash cartridge, which was then attached to flash purification system loaded with 40 g flash silica column and was purified by flash chromatography (SiO₂: ethyl acetate/hexane 0-10%) to get Compound L4s-1 (2.38 g, 90%). ¹H NMR (300 MHz, CDCl₃): δ ppm 3.78-3.72 (m, 2H), 3.00 (s, 2H), 1.62-1.14 (m, 6H), 0.99 (t, J=7.0 Hz, 3H), 0.68-0.56 (m, 4H), 0.28-0.25 (m, 2H), −0.25-0.29 (m, 1H); CIMS m/z [M+H]⁺ 168.28.

### Synthesis of 4,4-bis(2-(2-((2-ethylcyclopropyl)methyl)cyclopropyl)ethoxy)butanenitrile (L4s-2)

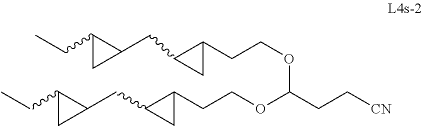 [SMILES: CCC1CC1CC1CC1CCOC(CCC#N)OCCC1CC1CC1CC1CC]

Prepared following Procedure A described in Compound 77 synthesis. Compound L4s-2 (1.88 g, 86%); ¹H NMR (300 MHz, CDCl₃): δ ppm 4.58 (t, J=5.0 Hz, 1H), 3.70-3.66 (m, 2H), 3.53-3.48 (m, 2H), 2.43 (t, J=7.0 Hz, 2H), 1.99-1.94 (m, 2H), 1.56-1.18 (m, 14H), 0.96 (t, J=7.0 Hz, 6H), 0.62-0.52 (m, 8H), 0.26-0.21 (m, 4H), −0.25-0.29 (m, 2H); CIMS m/z [M+H]⁺ 402.75.

### Synthesis of 4,4-bis(2-(2-((2-ethylcyclopropyl)methyl)cyclopropyl)ethoxy)butanoic acid (L4s-3)

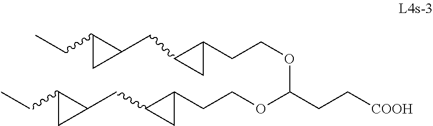 [SMILES: CCC1CC1CC1CC1CCOC(CCC(=O)O)OCCC1CC1CC1CC1CC]

Prepared following Procedure B described in Compound 77 synthesis. Compound L4s-3 (1.71 g, 91%) was isolated as colorless oil. ¹H NMR (300 MHz, CDCl₃): δ ppm ppm 4.56 (t, J=5.0 Hz, 1H), 3.70-3.66 (m, 2H), 3.52-3.48 (m, 2H), 2.47 (t, J=7.0 Hz, 2H), 1.99-1.94 (m, 2H), 1.50-1.17 (m, 14H), 0.96 (t, J=7.0 Hz, 6H), 0.62-0.52 (m, 8H), 0.25-0.20 (m, 4H), −0.28-0.31 (m, 2H); CIMS m/z [M+H]⁺ 421.72.

### Synthesis of 6-bromohexyl 4,4-bis(2-(2-((2-ethylcyclopropyl)methyl) cyclopropyl)ethoxy) butanoate (L4s-4)

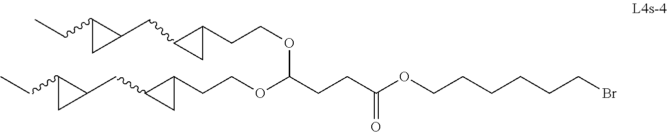 [SMILES: CCC1CC1CC1CC1CCOC(CCC(=O)OCCCCCCBr)OCCC1CC1CC1CC1CC]

Prepared following Procedure C described in Compound 77 synthesis. Compound L4s-4 (1.7 g, 67%) was isolated as a clear oil. ¹H NMR (300 MHz, CDCl₃): δ ppm 4.52 (t, J=5.0 Hz, 1H), 4.06 (t, J=7.0 Hz, 2H), 3.70-3.20 (m, 2H), 3.50-3.37 (m, 4H), 2.38 (t, J=7.0 Hz, 2H), 1.99-1.83 (m, 6H), 1.49-1.33 (m, 16H), 0.96 (t, J=7.0 Hz, 6H), 0.62-0.51 (m, 10H), 0.25-0.20 (m, 4H), −0.28-0.31 (m, 2H); CIMS m/z [M+H]⁺ 584.71.

### Synthesis of ((4-hydroxybutyl)azanediyl)bis(hexane-6,1-diyl) bis(4,4-bis(2-(2-((2-ethylcyclopropyl)methyl)cyclopropyl)ethoxy)butanoate) (Compound 129)

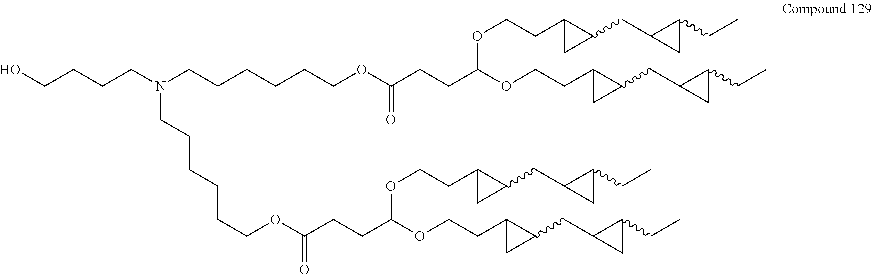 [SMILES: CCC1CC1CC1CC1CCOC(CCC(=O)OCCCCCCN(CCCCO)CCCCCCOC(=O)CCC(OCCC1CC1CC1CC1CC)OCCC1CC1CC1CC1CC)OCCC1CC1CC1CC1CC]

Prepared following Procedure D described in Compound 77 synthesis. Compound 129 (895 mg, 66%). ¹H NMR (300 MHz, CDCl₃): δ ppm 4.5 (t, J=5.0 Hz, 2H), 4.06 (t, J=5.0 Hz, 4H), 3.78-3.61 (m, 4H), 3.60-3.51 (m, 6H), 2.39 (m, 101H), 1.94 (m, 4H), 1.71-1.02 (m, 46H), 0.96 (t, J=7.0 Hz, 12H), 0.75-0.51 (m, 20H), 0.24-0.22 (m, 8H), −3.9-0.28 (m, 4H). CIMS m/z [M+H]⁺ 1095.82. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity:>99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=14.3 min, purity: >99%.

### Synthetic Scheme for Compound 66

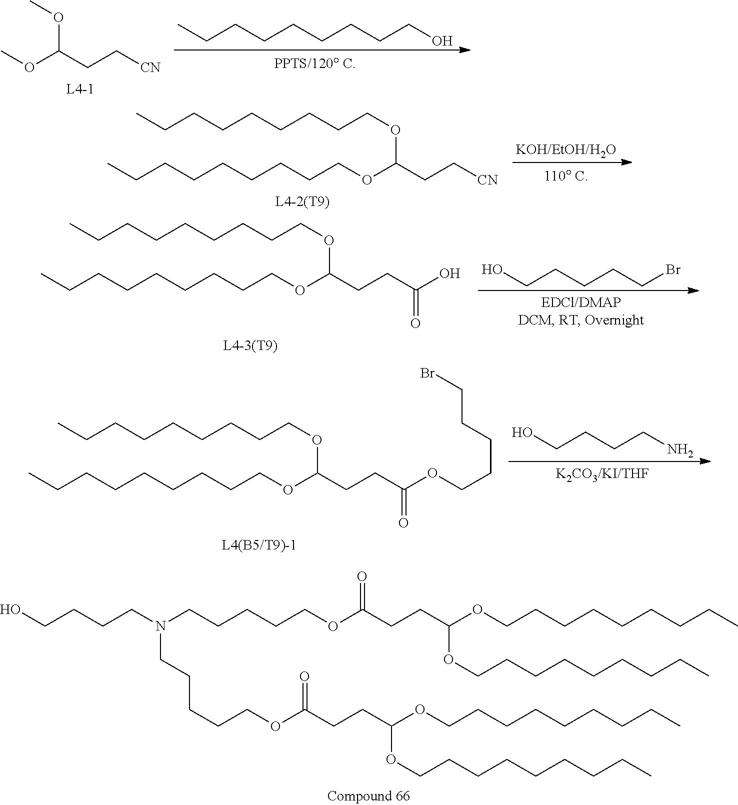

### Synthesis of 4,4-bis(nonyloxy)butanenitrile (L4-2(T9))

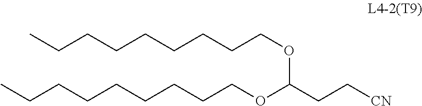 [SMILES: CCCCCCCCCOC(CCC#N)OCCCCCCCCC]

Prepared following Procedure A described in Compound 77 synthesis. Compound L4-2(T9) was isolated as light-yellow oil in a yield of 11.5 g (91%). ¹HNMR (CDCl₃) δ: 4.5-4.56 (t, 1H), 3.57-3.56 (m, 2H), 3.40-3.43 (m, 2H), 2.39-2.41 (t, 2H), 1.92-1.95 (m, 2H), 1.54-1.56 (m, 2H), 1.26 (bs, 26H), 0.85-0.87 (t, 6H); CIMS m/z [M+H]⁺ 354.

### Synthesis of 4,4-bis(nonyloxy)butanoic acid (L4-3(T9))

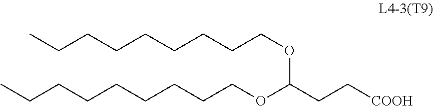 [SMILES: CCCCCCCCCOC(CCC(=O)O)OCCCCCCCCC]

Prepared following Procedure B described in Compound 77 synthesis. Compound L4-3(T9) was isolated as light-yellow oil in a yield of 11.8 g (98%). ¹HNMR (CDCl₃) δ: 4.53-4.56 (t, 1H), 3.57-3.60 (m, 2H), 3.40-3.43 (m, 2H), 2.39-2.41 (t, 2H), 1.90-1.95 (m, 2H), 1.54-1.56 (M, 4H), 1.26 (bs, 28H), 0.85-0.87 (t, 6H); CIMS m/z [M−H]⁻ 371.

### Synthesis of 5-bromopentyl 4,4-bis(nonyloxy)butanoate (L4(B5/T9)-1

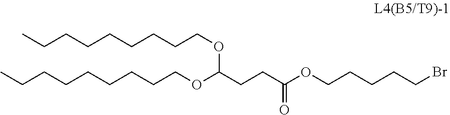 [SMILES: CCCCCCCCCOC(CCC(=O)OCCCCCBr)OCCCCCCCCC]

Prepared following Procedure C described in Compound 77 synthesis. Compound (L4(B5/T9)-1 was isolated as colorless oil (970 mg, 69%). ¹H-NMR (300 MHz, CDCl₃) δ: 4.48 (t, J=5.52 Hz, 1H), 4.07 (t, J=6.6 Hz, 2H), 3.55 (m, 2H), 3.4 (m, 4H), 2.38 (t, J=7.44 Hz, 2H), 1.83-1.95 (m, 4H), 1.65 (m, 2H), 1.47-1.55 (m, 6H), 1.25 (m, 24H), 0.87 (m, 6H).

### Synthesis of ((4-Hydroxybutyl)azanediyl)bis(pentane-5,1-diyl) bis(4,4-bis(nonyloxy) butanoate) (Compound 66)

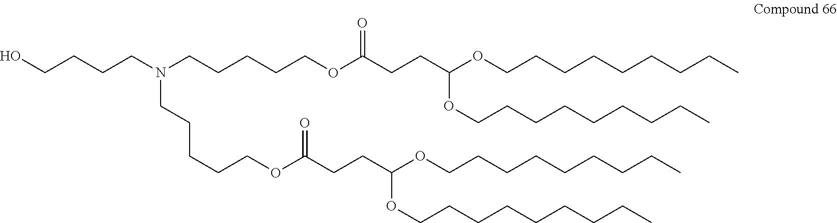 [SMILES: CCCCCCCCCOC(CCC(=O)OCCCCCN(CCCCO)CCCCCOC(=O)CCC(OCCCCCCCCC)OCCCCCCCCC)OCCCCCCCCC]

Prepared following Procedure D described in Compound 77 synthesis. Compound 66 was isolated as colorless oil (418 mg, 51%). H-NMR (300 MHz, CDCl₃) δ: 4.48 (t, J=5.49 Hz, 2H), 4.05 (t, J=6.6 Hz, 4H), 3.56 (m, 6H), 3.38 (m, 4H), 2.52 (brs, 2H), 2.37 (t, J=7.41 Hz, 4H), 1.9 (m, 4H), 1.52-1.66 (m, 20H), 1.25 (m, 56H), 0.87 (m, 12H); CIMS m/z [M+H]⁺ 970.7. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=9.4 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=15.2 min, purity: 98.6%.

### Synthetic Scheme for Compound 71

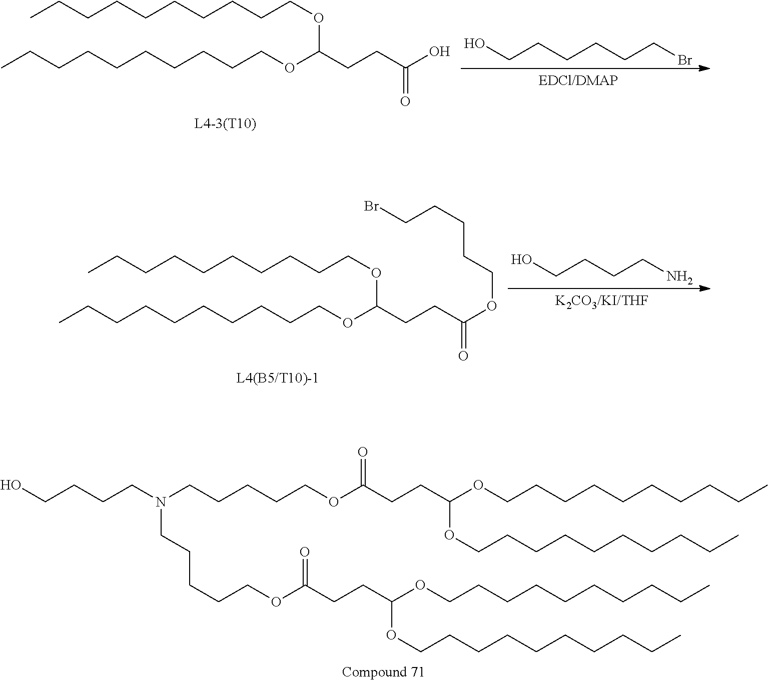

### Synthesis of 5-bromopentyl 4,4-bis(decyloxy)butanoate L4(B5/T10)-1

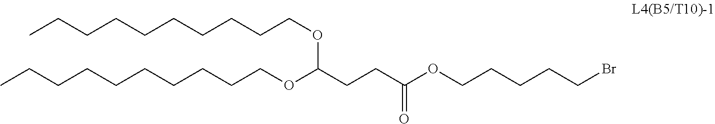 [SMILES: CCCCCCCCCCOC(CCC(=O)OCCCCCBr)OCCCCCCCCCC]

Prepared following Procedure C described in Compound 77 synthesis. Compound L4(B5/T10)-1 was isolated as colorless oil (1.0 g, 70%). ¹H-NMR (300 MHz, CDCl₃) δ: 4.48 (t, J=5.49 Hz, 1H), 4.07 (t, J=6.57 Hz, 2H), 3.54 (m, 2H), 3.4 (m, 4H), 2.38 (t, J=7.41 Hz, 2H), 1.85-1.95 (m, 4H), 1.63 (m, 2H), 1.47-1.55 (m, 8H), 1.25 (m, 26H), 0.87 (m, 6H).

### Synthesis of ((4-hydroxybutyl)azanediyl)bis(pentane-5,1-diyl) bis(4,4-bis(decyloxy) butanoate) (Compound 71)

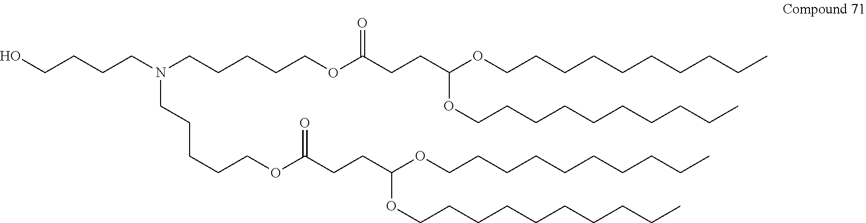 [SMILES: CCCCCCCCCCOC(CCC(=O)OCCCCCN(CCCCO)CCCCCOC(=O)CCC(OCCCCCCCCCC)OCCCCCCCCCC)OCCCCCCCCCC]

Prepared following Procedure D described in Compound 77 synthesis. Compound 71 was isolated as colorless oil (383 mg, 45%). ¹H-NMR (300 MHz, CDCl₃) δ: 4.48 (t, J=5.49 Hz, 2H), 4.05 (t, J=6.6 Hz, 4H), 3.56 (m, 6H), 3.4 (m, 4H), 2.6 (br, 2H), 2.37 (t, J=7.9 Hz, 4H), 1.9 (m, 4H), 1.52-1.67 (m, 20H), 1.25 (m, 64H), 0.87 (m, 12H); CIMS m/z [M+H]⁺ 1026.8. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=9.6 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=15.7 min, purity: >99%.

### Synthetic Scheme for Compound 74

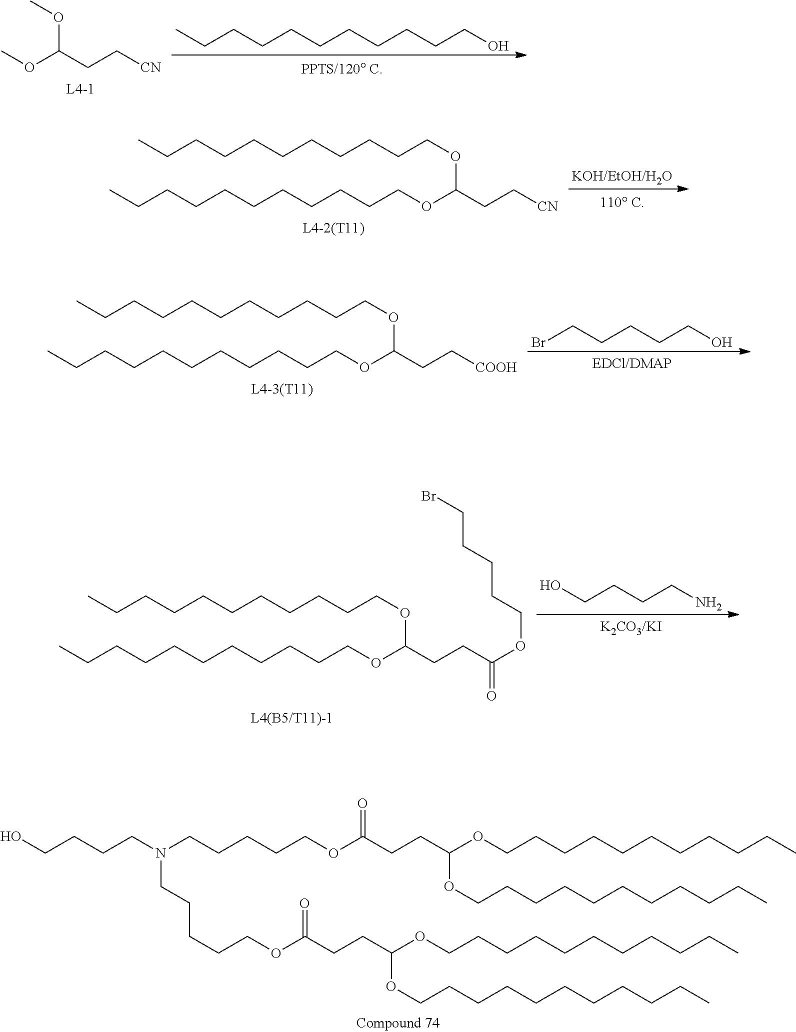

### Synthesis of 4,4-bis(nonyloxy)butanenitrile (L4-2(T11))

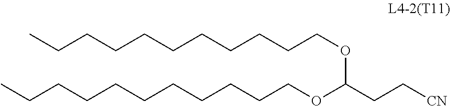 [SMILES: CCCCCCCCCCCOC(CCC#N)OCCCCCCCCCCC]

Prepared following Procedure A described in Compound 77 synthesis. Compound L4-2(T11) was isolated as colorless oil (4.5 g, 90%). ¹H-NMR (300 MHz, CDCl₃) δ 4.54 (t, 1H), 3.60-3.55 (m, 2H), 3.45-3.40 (m, 2H), 2.41 (t, 2H), 1.96-1.90 (m, 2H), 1.58-1.53 (m, 4H), 1.39-1.15 (m, 32H), 0.87 (t, 6H); CIMS m/z [M+H]⁺ 410.

### Synthesis of 4,4-bis(nonyloxy)butanoic acid (L4-3(T11))

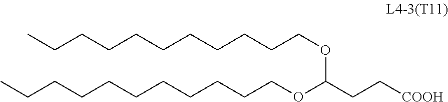 [SMILES: CCCCCCCCCCCOC(CCC(=O)O)OCCCCCCCCCCC]

Prepared following Procedure B described in Compound 77 synthesis. Compound L4-3(T11) was isolated as white solid (11.8 g, 98%). ¹H-NMR (300 MHz, CDCl₃) δ 4.50 (t, 1H), 3.82-3.52 (m, 2H), 3.41-3.38 (m, 2H), 2.41 (t, 2H), 1.95-1.88 (m, 2H), 1.57-1.50 (m, 4H), 1.39-1.18 (m, 32H), 0.87 (t, 6H); CIMS m/z [M−H]-426.7.

### Synthesis of (5-bromopentyl 4,4-bis(undecyloxy)butanoate) (L4(B5/T11)-1)

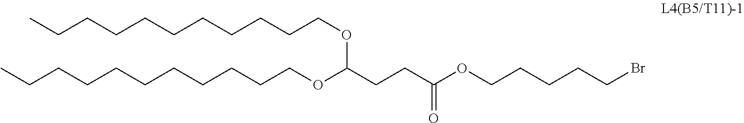 [SMILES: CCCCCCCCCCCOC(CCC(=O)OCCCCCBr)OCCCCCCCCCCC]

Prepared following Procedure C described in Compound 77 synthesis. L4-4(B5/T11)-1 was isolated as colorless oil (1.1 g, 82%). ¹H-NMR (300 MHz, CDCl₃) δ 4.48 (t, 1H), 4.05 (t, 2H), 3.54-3.37 (m, 6H), 2.37 (t, 2H), 1.92-1.86 (m, 3H), 1.59-1.36 (m, 8H), 1.35-1.15 (m, 33H), 0.87 (t, 6H); CIMS m/z [M+H]⁺ 578.6.

### Synthesis of ((4-hydroxybutyl)azanediyl)bis(pentane-5,1-diyl) bis(4,4-bis(undecyloxy) butanoate)) (Compound 74)

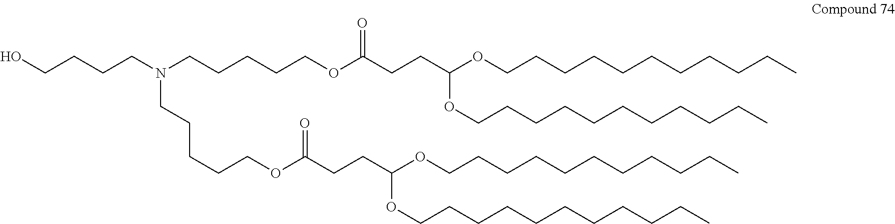 [SMILES: CCCCCCCCCCCOC(CCC(=O)OCCCCCN(CCCCO)CCCCCOC(=O)CCC(OCCCCCCCCCCC)OCCCCCCCCCCC)OCCCCCCCCCCC]

Prepared following Procedure D described in Compound 77 synthesis. Compound 74 was isolated as colorless oil (0.35 g, 41%). H-NMR (300 MHz, CDCl₃) δ 4.47 (t, 2H), 4.04 (t, 4H), 3.56-3.37 (m, 9H), 2.42-2.34 (m, 9H), 1.94-1.90 (m, 3H), 1.63-1.49 (m, 18H), 1.40-1.10 (m, 73H), 0.86 (t, 12H); CIMS m/z 1082.9 [M+H]. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=13.1 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=16.5 min, purity: 92.2%.

### Synthetic Scheme for Compound 67

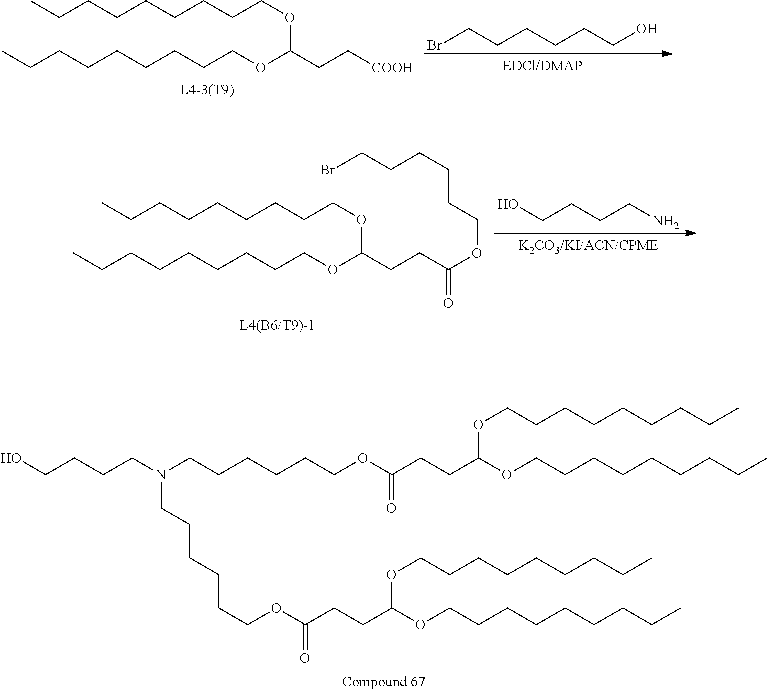

### Synthesis of 6-bromohexyl 4,4-bis(nonyloxy)butanoate (L4(B6/T9)-1)

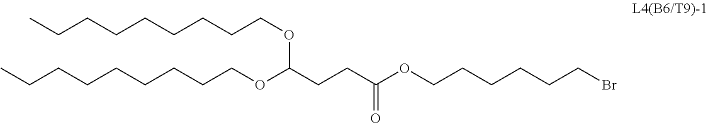 [SMILES: CCCCCCCCCOC(CCC(=O)OCCCCCCBr)OCCCCCCCCC]

Prepared following Procedure C described in Compound 77 synthesis. L4(B6/T9)-1 was isolated as colorless oil (1.4 g, 49%). ¹H-NMR (300 MHz, CDCl₃) δ 4.49 (t, 1H), 4.06 (t, 2H), 3.58-3.50 (m, 2H), 3.42-3.38 (m, 4H), 2.38 (t, 2H), 1.93-1.80 (m, 4H), 1.68-1.10 (m, 34H), 0.87 (t, 6H).

### Synthesis of ((4-hydroxybutyl)azanediyl)bis(hexane-6,1-diyl) bis(4,4-bis(nonyloxy)butanoate) (Compound 67)

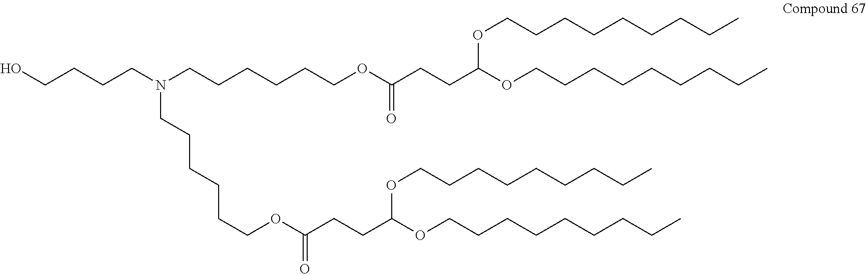 [SMILES: CCCCCCCCCOC(CCC(=O)OCCCCCCN(CCCCO)CCCCCCOC(=O)CCC(OCCCCCCCCC)OCCCCCCCCC)OCCCCCCCCC]

Prepared following Procedure D described in Compound 77 synthesis. Compound 67 was isolated as colorless oil (0.8 g, 61%). ¹H-NMR (300 MHz, CDCl₃) δ 4.49 (t, 2H), 4.04 (t, 4H), 3.60-3.50 (m, 6H), 3.43-3.36 (m, 4H), 2.45-2.35 (m, 10H), 1.95-1.88 (m, 4H), 1.68-1.42 (m, 26H), 1.40-1.10 (m, 62H), 0.87 (t, 12H); CIMS m/z [M+H]⁺ 999.2; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.95 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: mL/min, column temperature: 20±2° C., detector: CAD, t_{R}=15.27 min, purity: 97.54%.

### Synthetic Scheme for Compound 75

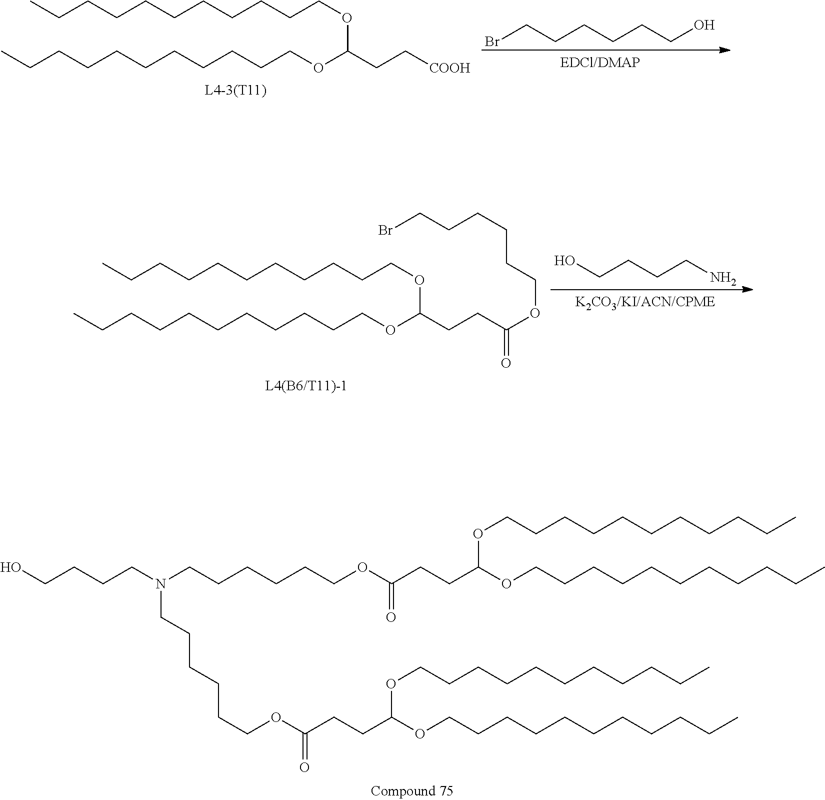

### Synthesis of (6-bromohexyl 4,4-bis(undecyloxy)butanoate) (L4(B6/T 11)-1

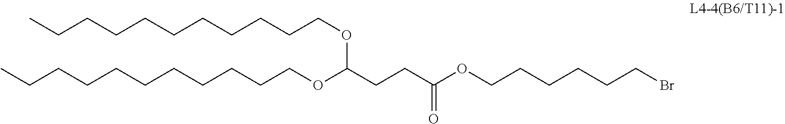 [SMILES: CCCCCCCCCCCOC(CCC(=O)OCCCCCCBr)OCCCCCCCCCCC]

Prepared following Procedure C described in Compound 77 synthesis. L4-4(B6/T11)-1 was isolated as colorless oil (1.08 g, 78%). H-NMR (300 MHz, CDCl₃) δ 4.47 (t, 1H), 4.07 (t, 2H), 3.53-3.36 (m, 6H), 2.35 (t, 2H), 1.91-1.84 (m, 3H), 1.57-1.35 (m, 9H), 1.33-1.17 (m, 34H), 0.87 (t, 6H); CIMS m/z 591.6 [M+H].

### Synthesis of ((4-hydroxybutyl)azanediyl)bis(hexane-6,1-diyl) bis(4,4-bis(undecyloxy)butanoate)) (75)

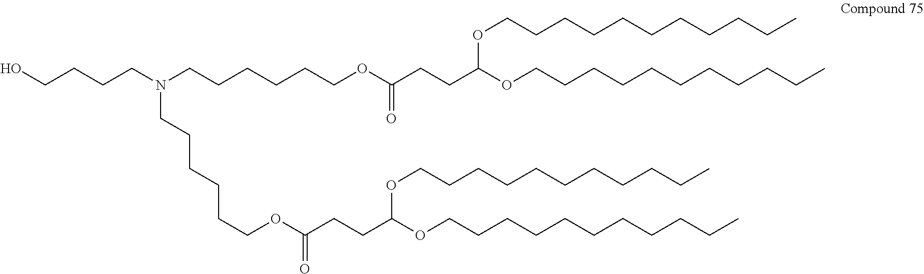 [SMILES: CCCCCCCCCCCOC(CCC(=O)OCCCCCCN(CCCCO)CCCCCCOC(=O)CCC(OCCCCCCCCCCC)OCCCCCCCCCCC)OCCCCCCCCCCC]

Prepared following Procedure D described in Compound 77 synthesis. Compound 75 was isolated as colorless oil (0.5 g, 57%). ¹H-NMR (300 MHz, CDCl₃) δ 4.48 (t, 2H), 4.03 (t, 4H), 3.57-3.37 (m, 9H), 2.41-2.34 (m, 9H), 1.94-1.90 (m, 3H), 1.63-1.52 (m, 18H), 1.37-1.15 (m, 77H), 0.86 (t, 12H); CIMS m/z 1110.9 [M+H]. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=13.0 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=16.7 min, purity: 93.5%.

### Synthetic Scheme for Compound 64

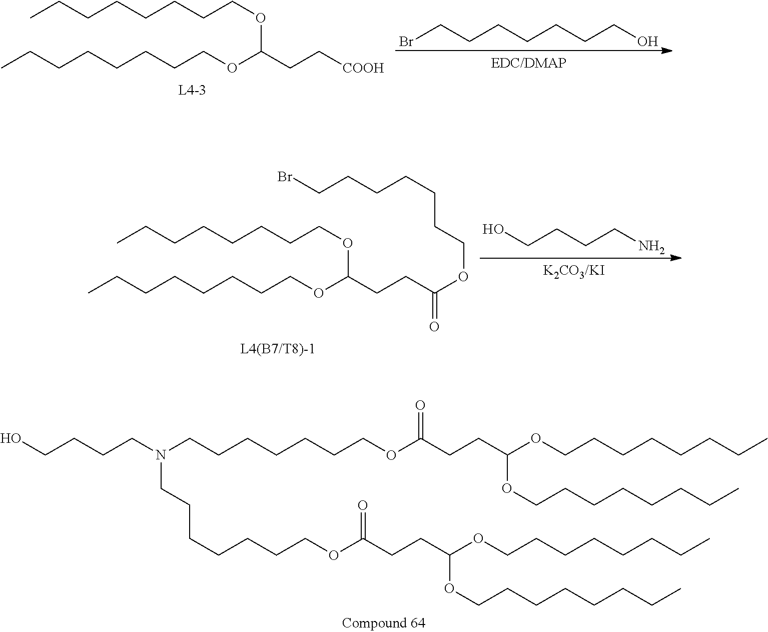

### Synthesis of 7-bromoheptyl 4,4-bis(octyloxy)butanoate [L4(B7/T8)-1]

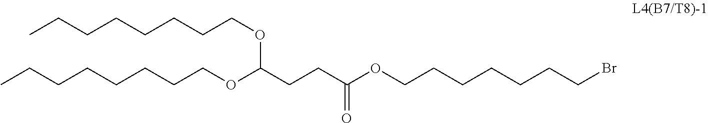 [SMILES: CCCCCCCCOC(CCC(=O)OCCCCCCCBr)OCCCCCCCC]

Prepared following Procedure C described in Compound 77 synthesis. Compound L4(B7/T8)-1 was isolated as light-yellow oil in a yield of 1.95 g (86%). ¹H-NMR (300 MHz, CDCl₃) δ 4.49 (t, 1H), 4.05 (t, 2H), 3.62-3.49 (m, 2H), 3.42-3.31 (m, 4H), 2.38 (t, 2H), 1.97-1.78 (m, 4H), 1.69-1.49 (m, 8H), 1.44-1.18 (m, 24H), 0.87 (t, 6H).

### Synthesis of ((4-hydroxybutyl)azanediyl)bis(heptane-7,1-diyl) bis(4,4-bis(octyloxy)butanoate) (64)

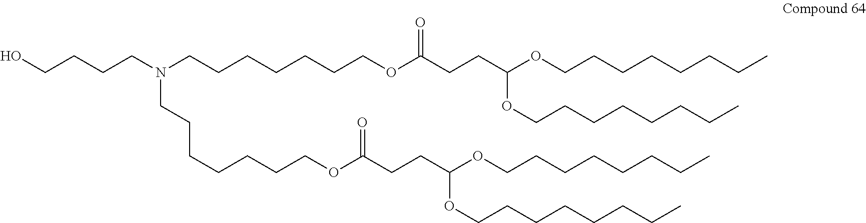 [SMILES: CCCCCCCCOC(CCC(=O)OCCCCCCCN(CCCCO)CCCCCCCOC(=O)CCC(OCCCCCCCC)OCCCCCCCC)OCCCCCCCC]

Prepared following Procedure D described in Compound 77 synthesis. Compound 64 was isolated as light-yellow oil in a yield of 940 mg (59%). ¹H-NMR (300 MHz, CDCl₃) δ 4.49 (t, 2H), 4.05 (t, 4H), 3.66-3.49 (m, 6H), 3.45-3.34 (m, 4H), 2.92-2.46 (m, 6H), 2.38 (t, 4H), 1.97-1.86 (m, 4H), 1.79-1.42 (m, 22H), 1.40-1.12 (m, 51H), 0.87 (t, 12H); CIMS m/z [M+H]⁺ 971.9. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.70 min, purity: >99.9%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=6.68 min, purity: 99.8%.

### Synthetic Scheme for Compound 68

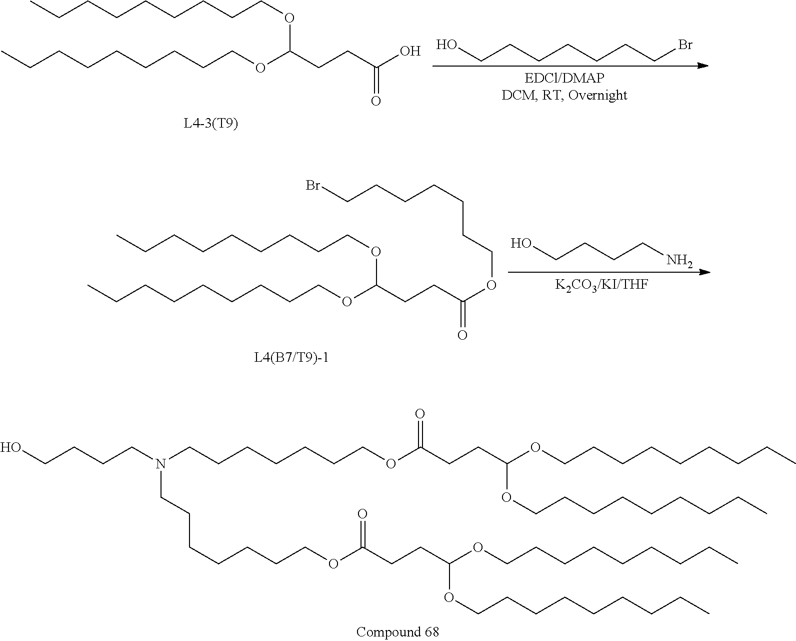

### Synthesis of 7-bromoheptyl 4,4-bis(nonyloxy)butanoate L4(B7/T9)-1

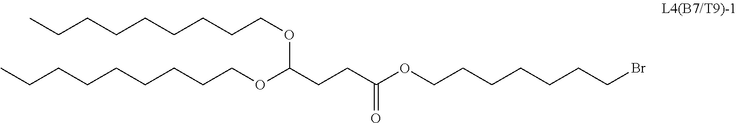 [SMILES: CCCCCCCCCOC(CCC(=O)OCCCCCCCBr)OCCCCCCCCC]

Prepared following Procedure C described in Compound 77 synthesis. L4(B7/T9)-1 was isolated as colorless oil (960 mg, 65%). ¹H-NMR (300 MHz, CDCl₃) δ: 4.48 (t, J=5.49 Hz, 1H), 4.05 (t, J=6.6 Hz, 2H), 3.56 (m, 2H), 3.4 (m, 4H), 2.37 (t, J=7.41 Hz, 2H), 1.82-1.95 (m, 4H), 1.52-1.62 (m, 6H), 1.43 (m, 2H), 1.25-1.35 (m, 28H), 0.87 (m, 6H).

### Synthesis of ((4-hydroxybutyl)azanediyl)bis(heptane-7,1-diyl) bis(4,4-bis(nonyloxy) butanoate) (68)

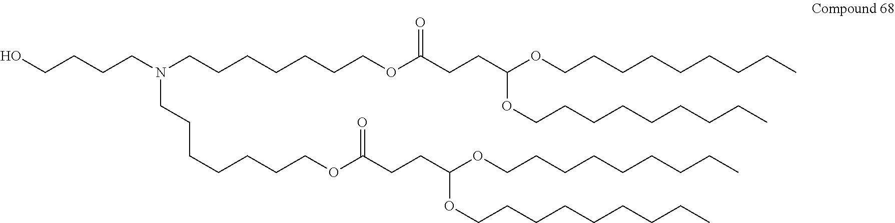 [SMILES: CCCCCCCCCOC(CCC(=O)OCCCCCCCN(CCCCO)CCCCCCCOC(=O)CCC(OCCCCCCCCC)OCCCCCCCCC)OCCCCCCCCC]

Prepared following Procedure D described in Compound 77 synthesis. Compound 68 was isolated as colorless oil (425 mg, 52%). ¹H-NMR (300 MHz, ¹H-NMR (300 MHz, CDCl₃) δ: 4.48 (t, J=5.76 Hz, 2H), 4.04 (t, J=6.5 Hz, 4H), 3.56 (m, 6H), 3.38 (m, 4H), 2.46 (br, 2H), 2.38 (t, J=7.4 Hz, 4H), 1.92 (m, 4H), 1.5-1.65 (m, 20H), 1.25 (m, 60H), 0.87 (m, 12H); CIMS m/z [M+H]⁺ 1026.9. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.7 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=15.5 min, purity: 97.9%.

### Synthetic Scheme for Compound 72:

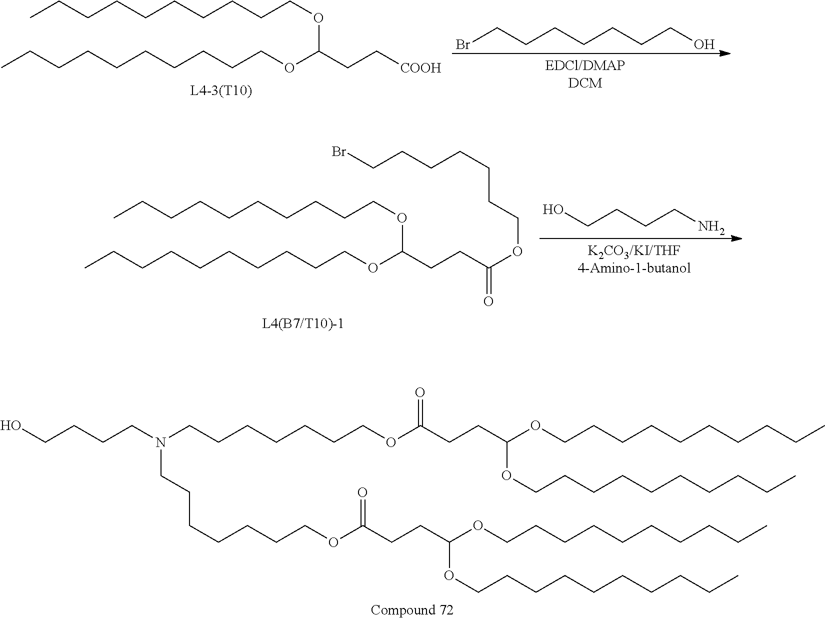

### Synthesis of 7-bromoheptyl 4,4-bis(decyloxy)butanoate (L4(B7/T10)-1)

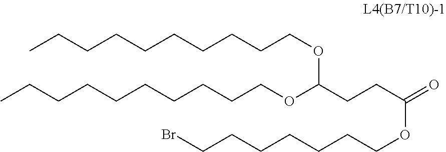 [SMILES: CCCCCCCCCCOC(CCC(=O)OCCCCCCCBr)OCCCCCCCCCC]

Prepared following Procedure C described in Compound 77 synthesis. Compound L4(B7/T10)-1 (1.37 g, 95%) was isolated as slightly yellow oil. ¹H NMR (300 MHz, CDCl₃): δ ppm 4.49 (t, J=7.0 Hz, 1H), 4.05 (t, J=7.0 Hz, 1H), 3.65-3.50 (m, 2H), 3.42-3.37 (m, 4H), 2.35 (t, J=7.0 Hz, 2H), 1.95-1.82 (m, 4H), 1.61-1.19 (m, 40H), 0.87 (s, 6H). CIMS m/z [M+H]⁺ 578.77.

### Synthesis of ((4-hydroxybutyl)azanediyl)bis(heptane-7,1-diyl) bis(4,4-bis(decyloxy) butanoate) (72)

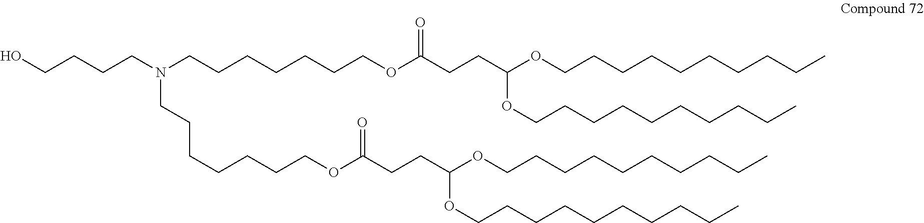 [SMILES: CCCCCCCCCCOC(CCC(=O)OCCCCCCCN(CCCCO)CCCCCCCOC(=O)CCC(OCCCCCCCCCC)OCCCCCCCCCC)OCCCCCCCCCC]

Prepared following Procedure D described in Compound 77 synthesis. Compound 72 (950 mg, 78%). ¹H NMR (300 MHz, CDCl₃): d ppm 4.49 (t, J=7.0 Hz, 2H), 4.04 (t, J=7.0 Hz, 4H), 3.75-3.350 (m, 10H), 2.42-2.30 (m, 10H), 1.95-1.82 (m, 4H), 1.61-1.35 (m, 20H), 1.26 (bs, 69H), 0.87 (t, J=6.0 Hz, 12H).; CIMS m/z [M+H]⁺ 1083.77. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=16.1 min, purity: >99%.

### Synthetic Scheme for Compound 69

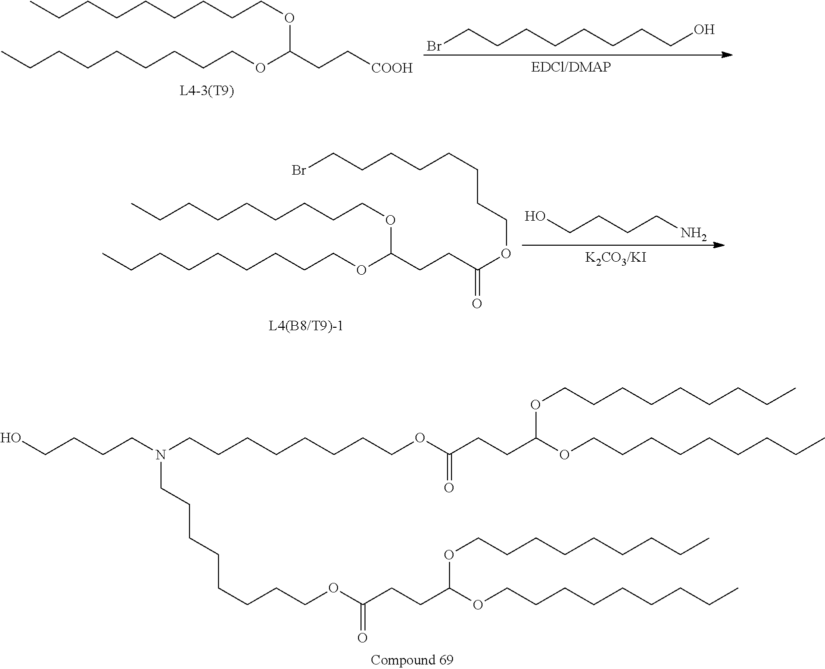

### Synthesis of (8-bromooctyl 4,4-bis(nonyloxy)butanoate) (L4(B8/T9-1)

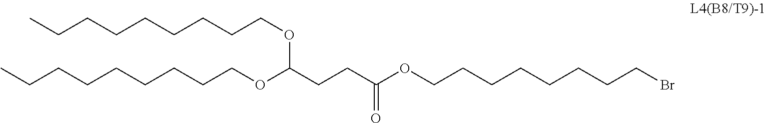 [SMILES: CCCCCCCCCOC(CCC(=O)OCCCCCCCCBr)OCCCCCCCCC]

Prepared following Procedure C described in Compound 77 synthesis. L4-4(B8/T9)-1 was isolated as colorless oil (1.3 g, 86%). ¹H-NMR (300 MHz, CDCl₃) δ 4.48 (t, 1H), 4.04 (t, 2H), 3.57-3.35 (m, 5H), 2.36 (t, 2H), 1.92-1.79 (m, 4H), 1.68-1.52 (m, 6H), 1.47-1.15 (m, 33H), 0.86 (t, 6H); CIMS m/z 563.56 [M+H].

### Synthesis of ((4-hydroxybutyl)azanediyl)bis(octane-8,1-diyl) bis(4,4-bis(nonyloxy)butanoate) (69)

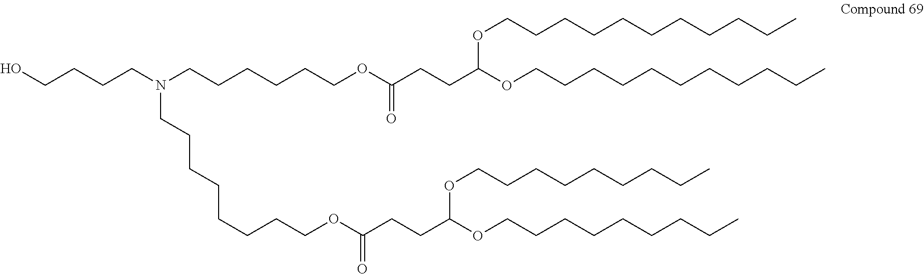 [SMILES: CCCCCCCCCCCOC(CCC(=O)OCCCCCCN(CCCCO)CCCCCCCCOC(=O)CCC(OCCCCCCCCC)OCCCCCCCCC)OCCCCCCCCCCC]

Prepared following Procedure D described in Compound 77 synthesis. Compound L4(B8/T9) was isolated as colorless oil (0.65 g, 73%). ¹H-NMR (300 MHz, CDCl₃) δ 6.70 (s, 1H), 4.48 (t, 2H), 4.03 (t, 4H), 3.57-3.37 (m, 10H), 2.40-2.36 (m, 9H), 1.92-1.89 (m, 3H), 1.56-1.52 (m, 19H), 1.41-1.12 (m, 67H), 0.86 (t, 12H); CIMS m/z 1054.8 [M+H]. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.8 min, purity: 94.9%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=15.6 min, purity: 82.9%.

### Synthetic Scheme for Compound 73

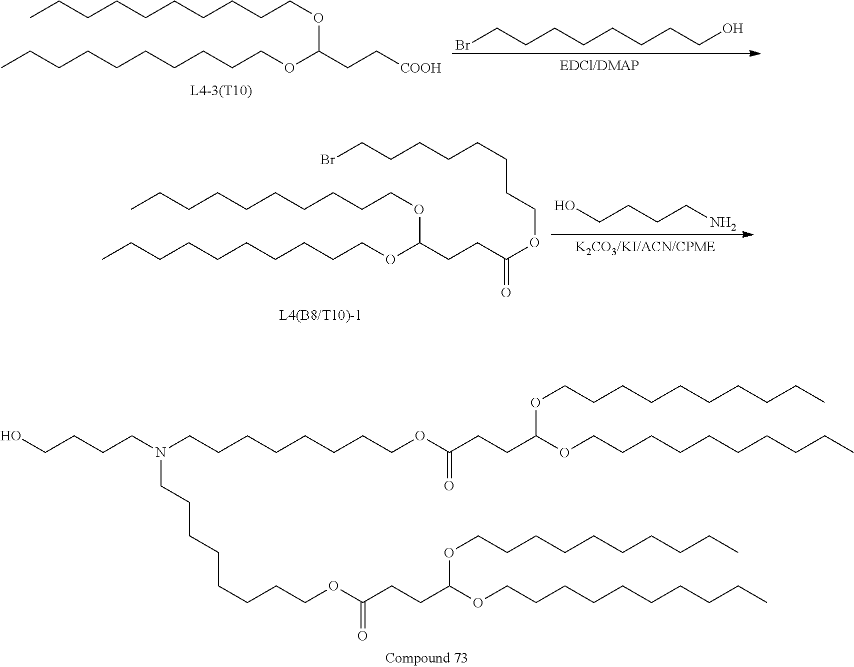

### Synthesis of 8-bromooctyl 4,4-bis(decyloxy)butanoate (L4(B8/T10)-1)

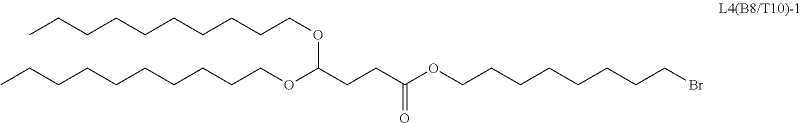 [SMILES: CCCCCCCCCCOC(CCC(=O)OCCCCCCCCBr)OCCCCCCCCCC]

Prepared following Procedure C described in Compound 77 synthesis. Compound L4(B8/T10)-1 was isolated as colorless oil in a yield of 980 mg (67%). ¹H-NMR (300 MHz, CDCl₃) δ 4.49 (t, 1H), 4.05 (t, 2H), 3.62-3.49 (m, 2H), 3.42-3.31 (m, 4H), 2.38 (t, 2H), 1.97-1.78 (m, 4H), 1.69-1.49 (m, 8H), 1.44-

### Synthesis of ((4-hydroxybutyl)azanediyl)bis(octane-8,1-diyl) bis(4,4-bis(decyloxy)butanoate) (73)

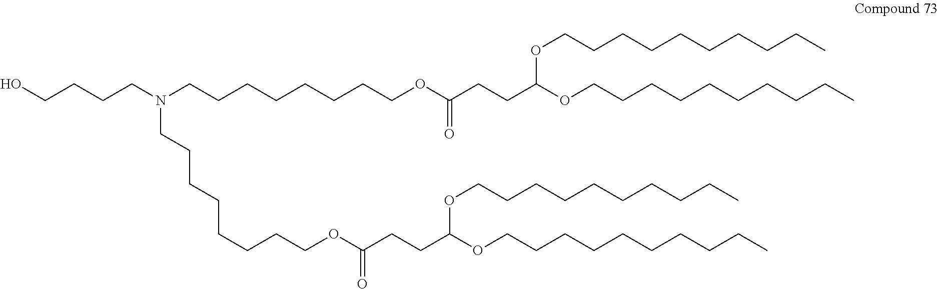 [SMILES: CCCCCCCCCCOC(CCC(=O)OCCCCCCCCN(CCCCO)CCCCCCCCOC(=O)CCC(OCCCCCCCCCC)OCCCCCCCCCC)OCCCCCCCCCC]

Prepared following Procedure D described in Compound 77 synthesis. Compound 73 was isolated as light-yellow oil in a yield of 442 mg (63%). ¹H-NMR (300 MHz, CDCl₃) δ 4.49 (t, 2H), 4.05 (t, 4H), 3.67 (m, 2H), 3.59-3.49 (m, 4H), 3.44-3.31 (m, 4H), 3.18-2.51 (m, 6H), 2.36 (t, 4H), 1.97-1.78 (m, 6H), 1.74-1.44 (m, 24H), 1.38-1.12 (m, 78H), 0.87 (t, 12H); CIMS m/z [M+H]⁺ 1111.9. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=7.48 min, purity: >99.9%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=7.72 min, purity: 99.6%.

### Synthetic Scheme for Compound 65

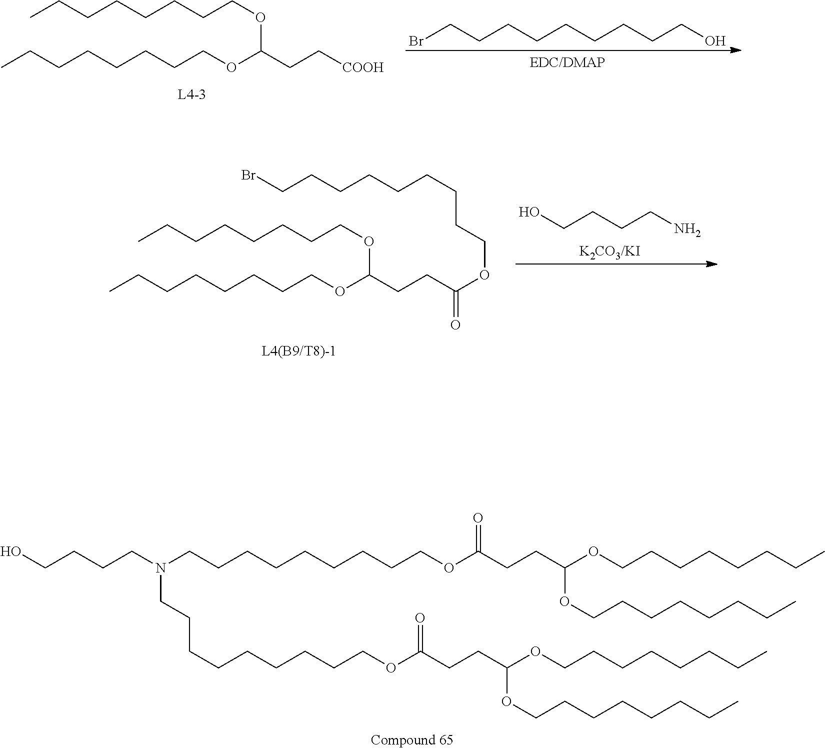 [SMILES: CCCCCCCCOC(CCC(=O)O)OCCCCCCCC ; OCCCCCCCCCBr ; CCCCCCCCOC(CCC(=O)OCCCCCCCCCBr)OCCCCCCCC ; NCCCCO ; CCCCCCCCOC(CCC(=O)OCCCCCCCCCN(CCCCO)CCCCCCCCCOC(=O)CCC(OCCCCCCCC)OCCCCCCCC)OCCCCCCCC]

### Synthesis of 9-bromononyl 4,4-bis(octyloxy)butanoate (L4(B9/T8)-1

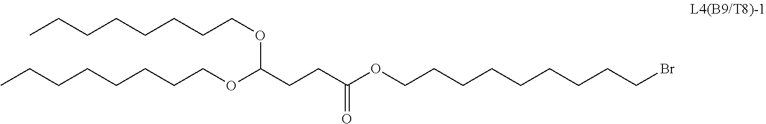 [SMILES: CCCCCCCCOC(CCC(=O)OCCCCCCCCCBr)OCCCCCCCC]

Prepared following Procedure C described in Compound 77 synthesis. Compound L4(B9/T8)-1 was isolated as light-yellow oil in a yield of 2.0 g (83%). ¹H-NMR (300 MHz, CDCl₃) δ 4.49 (t, 1H), 4.05 (t, 2H), 3.62-3.49 (m, 2H), 3.42-3.31 (m, 4H), 2.38 (t, 2H), 1.97-1.78 (m, 4H), 1.69-1.49 (m, 6H), 1.44-1.18 (m, 30H), 0.87 (t, 6H).

### Synthesis of ((4-hydroxybutyl)azanediyl)bis(nonane-9,1-diyl) bis(4,4-bis(octyloxy)butanoate) (65)

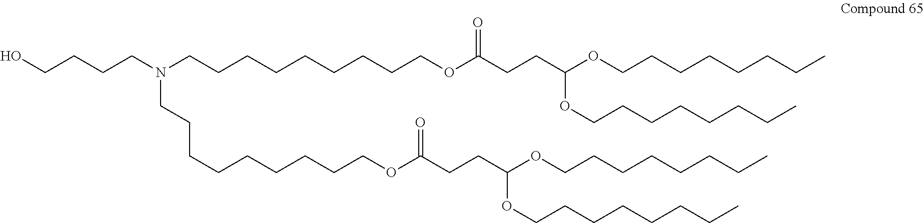 [SMILES: CCCCCCCCOC(CCC(=O)OCCCCCCCCCN(CCCCO)CCCCCCCCCOC(=O)CCC(OCCCCCCCC)OCCCCCCCC)OCCCCCCCC]

Prepared following Procedure D described in Compound 77 synthesis. Compound 65 was isolated as light-yellow oil in a yield of 880 mg (52%). ¹H-NMR (300 MHz, CDCl₃) δ 4.49 (t, 2H), 4.05 (t, 4H), 3.68-3.49 (m, 6H), 3.45-3.34 (m, 4H), 2.96-2.46 (m, 6H), 2.37 (t, 4H), 1.97-1.86 (m, 4H), 1.84-1.42 (m, 24H), 1.40-1.12 (m, 57H), 0.87 (t, 12H); CIMS m/z [M+H]⁺ 1027.9. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.78 min, purity: >99.9%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=7.17 min, purity: 99.1%.

### Synthetic Scheme for Compound 70:

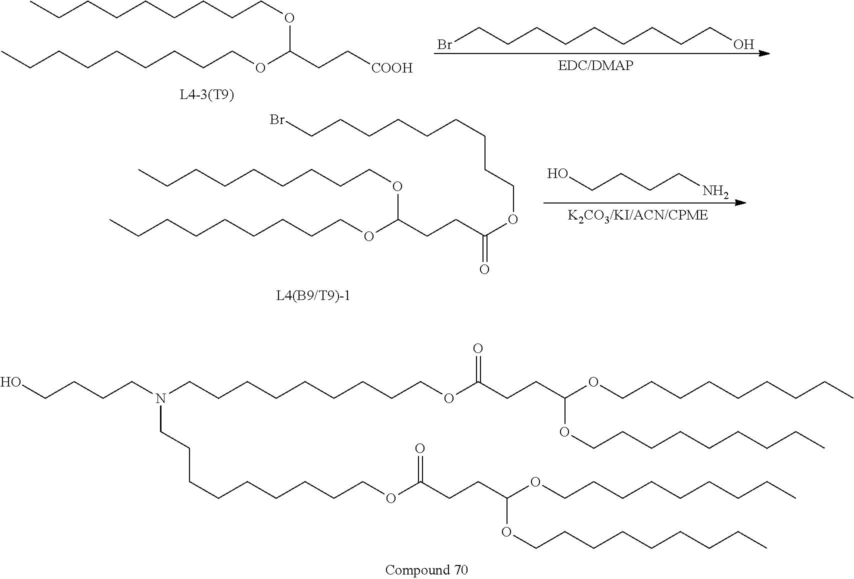 [SMILES: CCCCCCCCCOC(CCC(=O)O)OCCCCCCCCC ; OCCCCCCCCCBr ; CCCCCCCCCOC(CCC(=O)OCCCCCCCCCBr)OCCCCCCCCC ; NCCCCO ; CCCCCCCCCOC(CCC(=O)OCCCCCCCCCN(CCCCO)CCCCCCCCCOC(=O)CCC(OCCCCCCCCC)OCCCCCCCCC)OCCCCCCCCC]

### Synthesis of 9-bromononyl 4,4-bis(nonyloxy)butanoate (L4(B9/T9)-1)

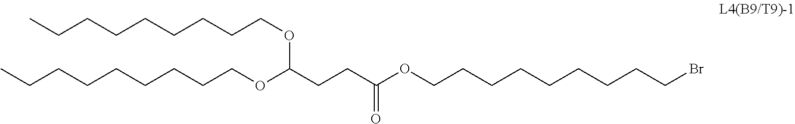 [SMILES: CCCCCCCCCOC(CCC(=O)OCCCCCCCCCBr)OCCCCCCCCC]

Prepared following Procedure C described in Compound 77 synthesis. Compound L4(B9/T9)-1 (810 mg, 52.25%) as a clear oil. ¹H NMR (300 MHz, CDCl₃): δ 4.48 (t, J=7.0 Hz, IH), 4.05 (t, J=7.0 Hz, 2H), 3.78-3.34 (m, 8H), 2.37 (t, J=7.0 Hz, 2H), 1.93-1.80 (m, 4H), 1.65-1.52 (m, 8H), 1.44-1.25 (m, 46H), 0.94-0.83 (m, 6H); CIMS m/z [M+H]⁺ 578.87.

### Synthesis of ((4-hydroxybutyl)azanediyl)bis(nonane-9,1-diyl) bis(4,4-bis(nonyloxy) butanoate) (70)

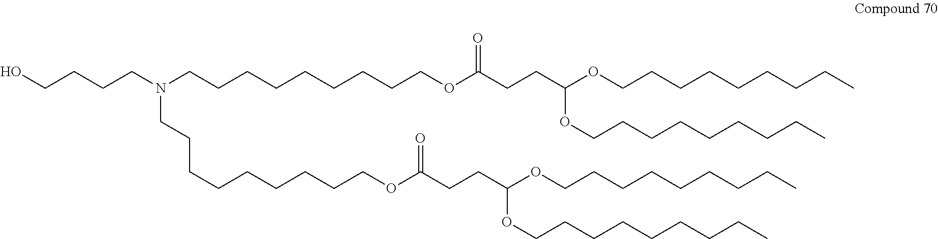 [SMILES: CCCCCCCCCOC(CCC(=O)OCCCCCCCCCN(CCCCO)CCCCCCCCCOC(=O)CCC(OCCCCCCCCC)OCCCCCCCCC)OCCCCCCCCC]

Prepared following Procedure D described in Compound 77 synthesis. Compound 70 (1.1 g, 89%). ¹H NMR (300 MHz, CDCl₃): δ 4.48 (t, J=7.0 Hz, 2H), 4.05 (t, J=7.0 Hz, 4H), 3.70-3.34 (m, 10H), 2.42-2.34 (m, 10H), 1.93-1.80 (m, 4H), 1.65-1.52 (m, 20H), 1.42-1.23 (m, 71H), 0.90-0.83 (m, 12H); CIMS m/z [M+H]⁺ 1083.77. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: I mL/min, column temperature: 20±2° C., detector: ELSD, t_{R}=10.7 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: I mL/min, column temperature: 20±2° C., detector: CAD, t_{R}=15.9 min, purity: 98.08%.

### Synthetic Scheme for Compound L4 (B10/T8) (Compound 76)

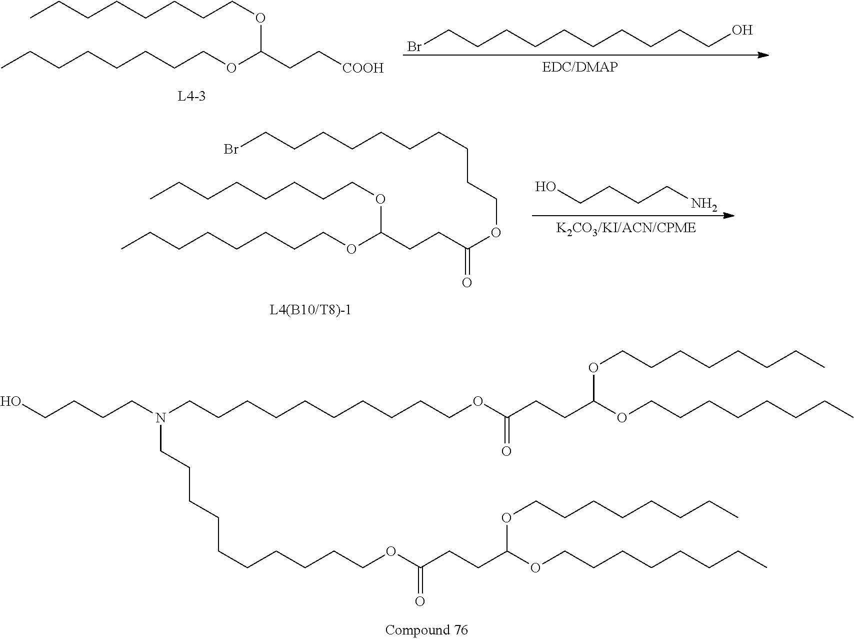 [SMILES: CCCCCCCCOC(CCC(=O)O)OCCCCCCCC ; OCCCCCCCCCCBr ; CCCCCCCCOC(CCC(=O)OCCCCCCCCCCBr)OCCCCCCCC ; NCCCCO ; CCCCCCCCOC(CCC(=O)OCCCCCCCCCCN(CCCCO)CCCCCCCCCCOC(=O)CCC(OCCCCCCCC)OCCCCCCCC)OCCCCCCCC]

### Synthesis of 6-bromohexyl 4,4-bis(hexyloxy)butanoate Compound (L4(B10/T8)-1)

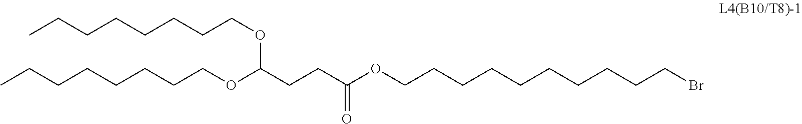 [SMILES: CCCCCCCCOC(CCC(=O)OCCCCCCCCCCBr)OCCCCCCCC]

Prepared following Procedure C described in Compound 77 synthesis. Compound L4(B10/T8)-1 (1.6 g, 68%) was isolated as light-yellow oil. ¹H NMR (300 MHz, CDCl₃): δ ppm 4.48 (t, 1H), 4.04 (t, 2H), 3.54-3.37 (m, 6H), 2.39-2.34 (m, 2H), 1.92-1.84 (m, 4H), 1.61-1.51 (m, 6H), 1.51-0.99 (m, 32H), 0.88-0.84 (m, 6H).

### Synthesis of ((4-hydroxybutyl)azanediyl)bis(decane-10,1-diyl) bis(4,4-bis(octyloxy) butanoate) (76)

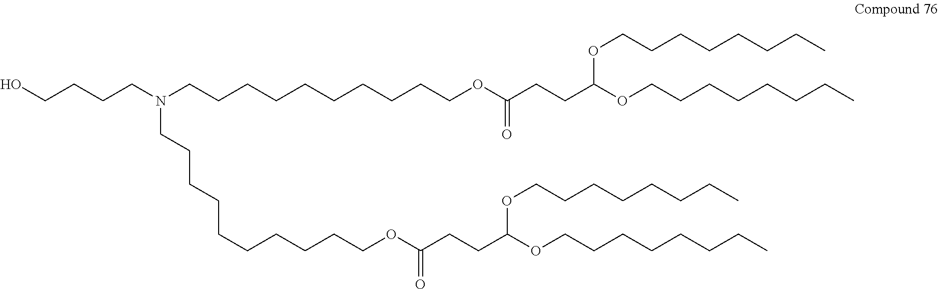 [SMILES: CCCCCCCCOC(CCC(=O)OCCCCCCCCCCN(CCCCO)CCCCCCCCCCOC(=O)CCC(OCCCCCCCC)OCCCCCCCC)OCCCCCCCC]

Prepared following Procedure D described in Compound 77 synthesis. Compound 76 (322 mg, 32%) was isolated as light-yellow oil. ¹H-NMR (300 MHz, CDCl₃) δ 4.48 (t, 2H), 4.01-4.06 (m, 4H), 3.57-3.40 (m, 10H), 2.39-2.34 (m, 10H), 1.92-1.90 (m, 4H), 1.65-1.51 (m, 18H), 1.30-0.99 (m, 66H), 0.86 (t, 12H); APCI-MS: m/z [M+H]⁺ 1055; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.6 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.8 min, purity: >99%.

### Synthetic Scheme for Compound 83

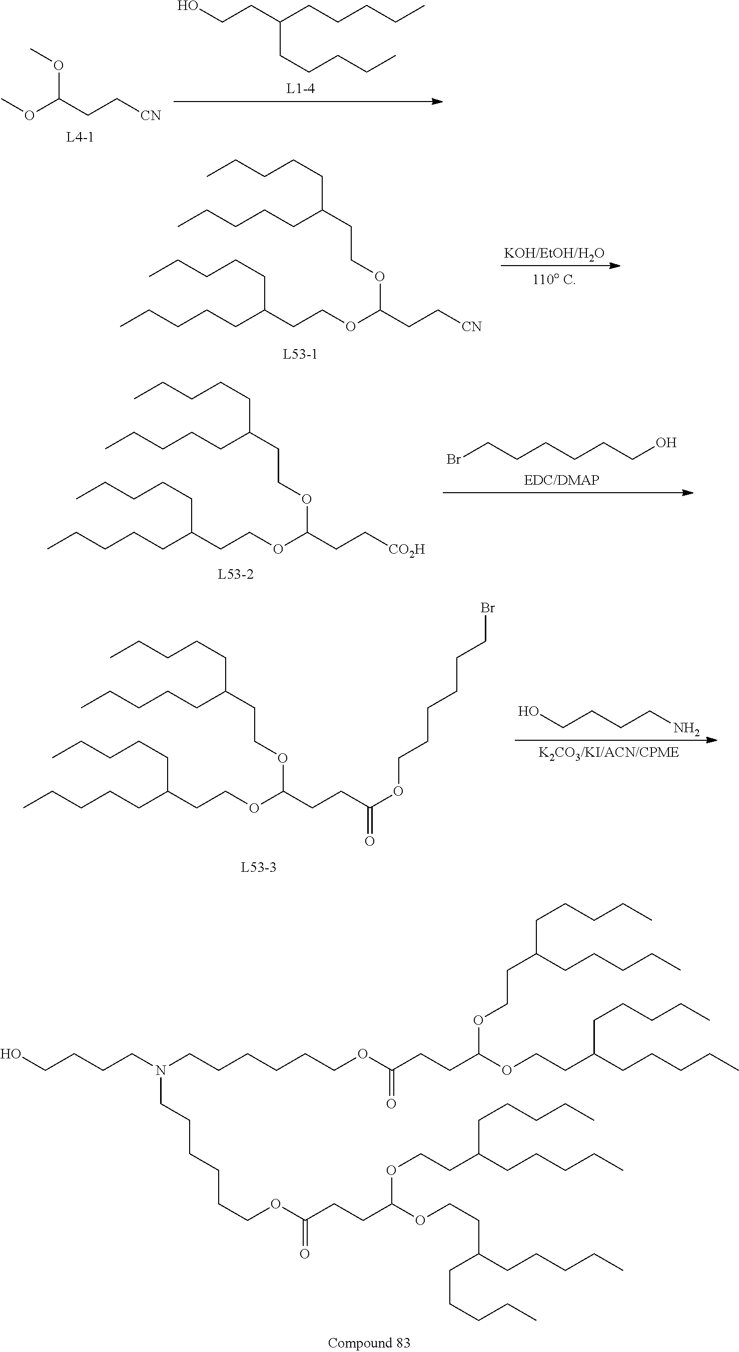

### Synthesis of 8-bromooctyl 4,4-bis((3-pentyloctyl)oxy)butanenitrile (L53-1)

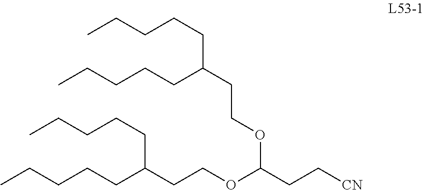 [SMILES: CCCCCC(CCCCC)CCOC(CCC#N)OCCC(CCCCC)CCCCC]

Prepared following Procedure A described in Compound 77 synthesis. L53-1 was isolated as light-yellow oil (3.66 g, 95%). ¹H-NMR (300 MHz, CDCl₃) δ 4.54 (t, 1H), 3.64-3.52 (m, 2H), 3.48-3.37 (m, 2H), 2.42 (t, 2H), 1.97-1.84 (m, 2H), 1.61-1.44 (m, 4H), 1.44-1.08 (m, 28H), 0.87 (m, 12H).

### Synthesis of 8-bromooctyl 4,4-bis((3-pentyloctyl)oxy)butanoic acid (L53-2)

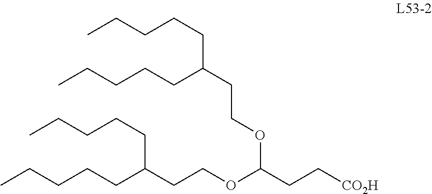 [SMILES: CCCCCC(CCCCC)CCOC(CCC(=O)O)OCCC(CCCCC)CCCCC]

Prepared following Procedure B described in Compound 77 synthesis. L53-2 was isolated as light-yellow oil (3.2 g, 84%). CIMS m/z [M−H]⁻ 683.

### Synthesis of 6-bromohexyl 4,4-bis((3-pentyloctyl)oxy)butanoate (L53-3)

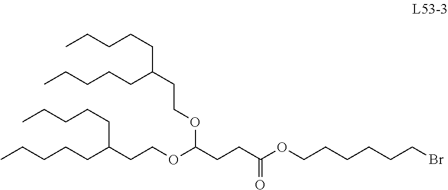 [SMILES: CCCCCC(CCCCC)CCOC(CCC(=O)OCCCCCCBr)OCCC(CCCCC)CCCCC]

Prepared following Procedure C described in Compound 77 synthesis. L53-3 was isolated as light-yellow oil (400 mg, 32%). ¹H-NMR (300 MHz, CDCl₃) δ 4.49 (t, 1H), 4.06 (t, 2H), 3.63-3.51 (m, 2H), 3.50-3.31 (m, 4H), 2.42 (t, 2H), 1.97-1.81 (m, 2H), 1.57-1.43 (m, 8H), 1.43-1.08 (m, 38H), 0.87 (m, 12H).

### Synthesis of ((4-hydroxybutyl)azanediyl)bis(hexane-6,1-diyl) bis(4,4-bis((3-pentyloctyl)oxy)butanoate) (83)

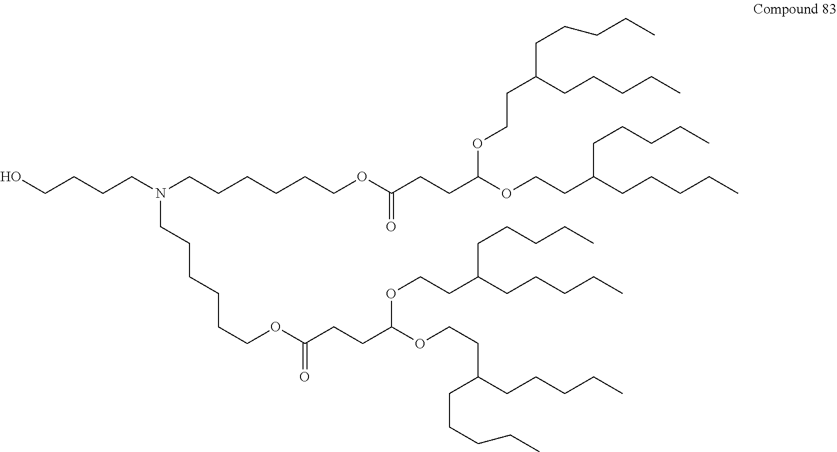 [SMILES: CCCCCC(CCCCC)CCOC(CCC(=O)OCCCCCCN(CCCCO)CCCCCCOC(=O)CCC(OCCC(CCCCC)CCCCC)OCCC(CCCCC)CCCCC)OCCC(CCCCC)CCCCC]

Prepared following Procedure D described in Compound 77 synthesis. Compound 83 was isolated as light-yellow oil (252 mg, 74%). ¹H-NMR (300 MHz, CDCl₃) δ 4.48 (t, 2H), 4.05 (t, 4H), 3.71-3.62 (m, 2H), 3.62-3.51 (m, 4H), 3.47-3.3.36 (m, 4H), 3.08-2.58 (m, 6H), 2.37 (t, 4H), 1.97-1.86 (m, 4H), 1.74-1.56 (m, 10H), 1.55-1.46 (m, 8H), 1.44-1.06 (m, 80H), 0.87 (m, 24H); CIMS m/z [M+H]⁺ 1223.2. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=8.33 min, purity: >99.9%; UPLC column: Waters Aquity UPLC® CSHTM, C18, 1.7 μm, 3.0×150 mum, (Part No. 186005302), mobile phase A: acetonitrile with 0.1% trifluoroacetic acid, mobile phase B: water with 0.1% trifluoroacetic acid, use gradient: A in B 80% to 100% in 15 min, then 100% for 5 min. Flow rate: 1 mL/min, column temperature: 20±2° C., detector: CAD, t_{R}=16.4 min, purity: 99.7%.

### Synthesis of Compound CY43 (1-(4-hydroxybutyl) pyrrolidine-3,4-diyl) bis(butane-4,1-diyl) bis(2-hexyldecanoate)

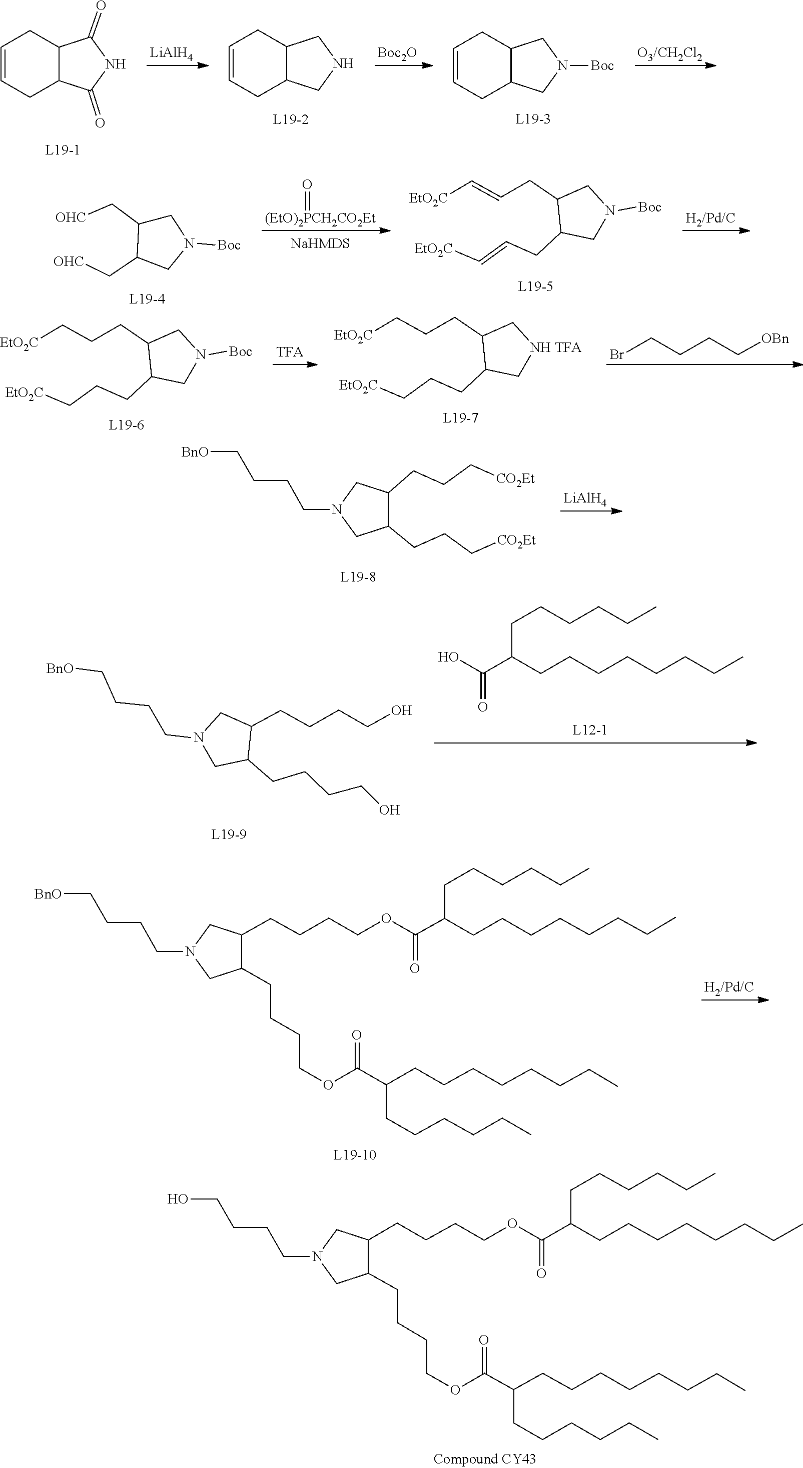

### Synthesis of 2,3,3a,4,7,7a-hexahydro-1H-isoindole (L19-2)

To a stirred solution of 3a,4,7,7a-tetrahydro-1H-isoindole-1,3(2H)-dione L19-1 (10 g, 66.1 mmol) in THF (200 mL) cooled to 0° C., 2 M lithium aluminum hydride in THF (82.5 mL, 165 mmol) was added dropwise. The reaction mixture was allowed to warm to room temperature and stirred for 12 h. After consumption of starting materials as observed by TLC, the reaction mixture was cooled to 0° C. and quenched with THF/water (40 mL, v/v 9:1) followed by 15% aq. solution of NaOH (40 mL) and water (100 mL) over 2 h. The resulting mixture was stirred at room temperature for 1 h and filtered through Celite followed by washing with DCM (3×100 mL). The collected filtrate was concentrated under reduced pressure to afford L19-2 (5.8 g, 71%) as brown liquid which was used for next step without further purification. CIMS m/z 124.2 [M+H]⁺.

### Synthesis of tert-butyl 1,3,3a,4,7,7a-hexahydro-2H-isoindole-2-carboxylate (L19-3)

A solution of crude L19-2 (5.8 g, 47.1 mmol) in THF (100 mL) was cooled to 0° C. under nitrogen. Triethylamine (9.8 mL, 70.6 mmol) and di-tert-butyl decarbonate (11.4 g, 52.2 mmol) were added, the reaction mixture was stirred at room temperature for 12 h. Water and DCM was added, and the aqueous phase was extracted with DCM. The organic extract was washed with saturated aqueous sodium bicarbonate and dried with Na₂SO₄. Filtration and concentration provided L19-3 as colorless oil (7.5 g, 71%). ¹H-NMR (300 MHz, CDCl₃) δ 5.62 (s, 2H), 3.42-3.33 (m, 2H), 3.17-3.03 (m, 2H), 2.30-2.16 (m, 4H), 1.91-1.85 (m, 2H), 1.44 (s, 9H).

### Synthesis of tert-butyl 3,4-bis(2-oxoethyl) pyrrolidine-1-carboxylate (L19-4)

L19-3 (3.0 g, 13.4 mmol, 1 eq) was dissolved in DCM (200 mL), and the solution was cooled to −78° C. Ozone was bubbled in until the color of the solution turned to blue. The reaction was then quenched with Dimethyl sulfide and stirred under nitrogen for 30 min. Removal of solvent under reduced pressure gave a crude material which was used for next step without further purification (2.31 g, 67%).

### Synthesis of diethyl 4,4′-(1-(tert-butoxycarbonyl) pyrrolidine-3,4-diyl) (2E,2′E)-bis(but-2-enoate) (L19-5)

To a solution of triethyl phosphonoacetate (11.2 g, 50.1 mmol) in THF (60 mL) cooled to −15° C. under nitrogen, was added dropwise of 1 M NaHMDS (10.1 mL, 50.1 mmol). After completion of addition, the mixture was stirred at the same temperature for 30 min then at 0° C. for 60 min. The resulted mixture was slowly added to crude L19-4 (3.2 g, 12.5 mmol) at 0° C. The reaction mixture was allowed to room temperature and stirred overnight. The reaction was quenched with aqueous ammonium chloride and extracted with ethyl acetate and dried over anhydrous Na₂SO₄. Filtration and concentration provided crude material which was purified by flash column chromatography (SiO₂: 0 to 35% ethyl acetate in hexane gradient) to yield L19-5 as colorless oil (1.01 g, 20%). ¹H-NMR (300 MHz, CDCl₃) δ 6.91-6.82 (m, 2H), 5.88-5.83 (m, 2H), 4.22-4.14 (m, 4H), 3.43-3.37 (m, 2H), 3.18-3.09 (m, 2H), 2.36-2.11 (m, 6H), 1.44 (s, 9H), 1.28 (t, 6H); CIMS m/z 296.1 [M-Boc+H]⁺.

### Synthesis of diethyl 4,4′-(1-(tert-butoxycarbonyl) pyrrolidine-3,4-diyl) dibutyrate (L19-6)

To a solution of compound L19-5 (0.58 g, 1.46 mmol) in ethyl acetate (20 mL), 10% P/C (0.2 g) was added. The mixture was stirred at room temperature under hydrogen balloon for 12 h and was filtered through a pad of Celite. After washed with ethyl acetate, the filtrates were concentrated, and crude was used for next step without further purification (0.57 g, 97%). ¹H-NMR (300 MHz, CDCl₃) δ 4.15-4.08 (m, 4H), 3.40-3.30 (m, 2H), 3.15-3.01 (m, 4H), 2.29 (t, 4H), 2.09-2.03 (m, 2H), 1.65-1.52 (m, 6H), 1.44 (s, 9H), 1.24 (t, 6H); CIMS m/z 300.2 [M-Boc+H]⁺.

### Synthesis of diethyl 4,4′-(pyrrolidine-3,4-diyl) dibutyrate TFA salt (L19-7)

To a solution of compound L19-6 (0.57 g, 1.42 mmol) in DCM (5 mL) was added TFA (5 mL) and the mixture was stirred at room temperature for 12 h. Volatile components were removed under reduced pressure and the crude product was used for next step without further purification (0.57 g, TFA salt). ¹H-NMR (300 MHz, CDCl₃) δ 4.15-4.08 (m, 4H), 3.37-3.10 (m, 4H), 2.35-2.30 (m, 7H), 1.61-1.43 (m, 7H), 1.24 (t, 6H); CIMS m/z 300.2 [M-Boc+H]⁺.

### Synthesis of diethyl 4,4′-(1-(4-(benzyloxy) butyl) pyrrolidine-3,4-diyl) dibutyrate (L19-8)

To a solution of compound L19-7 (460 mg, 1.5 mmol) and benzyl 4-bromobutyl ether (411 mg, 1.69 mmol) in CPME (5 mL) and ACN (5 mL) under nitrogen was added K₂CO₃ (850 mg, 6.1 mmol) and KI (255 mg, 1.53 mmol). The reaction mixture was heated at 60° C. for 18 h. After cooled to room temperature, the reaction mixture was filtered through Celite, washed with ethyl acetate, and the solvent removed under vacuum to give the crude product which was purified by flash chromatography. (40 g SiO₂: 0 to 10% methanol in dichloromethane gradient) to obtain compound L19-8 as colorless oil (0.41 g, 57%). ¹H-NMR (300 MHz, CDCl₃) δ 7.30-7.25 (m, 5H), 4.43 (s, 2H), 4.09-4.04 (m, 4H), 3.60-3.46 (m, 4H), 3.13-3.06 (m, 4H), 2.29 (t, 4H), 1.75-1.33 (m, 14H), 1.22 (t, 6H); CIMS m/z 462.2 [M+H]⁺.

### Synthesis of 4,4′-(1-(4-(benzyloxy) butyl) pyrrolidine-3,4-diyl) bis(butan-1-ol) (L199)

To a solution of compound L19-8 (0.4 g, 0.88 mmol) in THF (10 mL) cooled to 0° C. was added dropwise 1M lithium aluminum hydride in THF (1.1 mL, 1.1 mmol). The reaction mixture was allowed to room temperature and stirred for 12 h. After consumption of starting materials as observed by TLC, the reaction mixture was cooled to 0° C. and diluted with THF and quenched with 15% NaOH solution. The resulting mixture was stirred at room temperature for 1 h and filtered through Celite, followed by washing with ethyl acetate. The filtrates were concentrated to give crude product (0.21 g, 62%) which was used for next step without further purification. CIMS m/z 378.3 [M+H]⁺.

### Synthesis of (1-(4-(benzyloxy) butyl) pyrrolidine-3,4-diyl) bis(butane-4,1-diyl) bis(2-hexyldecanoate) (L19-10)

To a solution of compound L19-9 (200 mg, 0.53 mmol) in dichloromethane (6 mL) was added DMAP (65 mg, 0.53 mmol) and EDC (0.609 g, 3.18 mmol), followed by the addition of acid L12-1 (0.135 g, 0.53 mmol). The reaction mixture was stirred at room temperature for 24 h and evaporated under vacuum. The residue was dissolved in dichloromethane (100 mL) and washed with brine (80 mL×3). After dried over anhydrous Na₂SO₄, the solvent was evaporated, and the crude was purified by column chromatography (40 g SiO₂: 0 to 10% methanol in dichloromethane gradient) to obtain compound L19-10 as colorless oil. (0.41 g, 57%). ¹H-NMR (300 MHz, CDCl₃) δ 7.33-7.26 (m, 5H), 4.46 (s, 2H), 4.04 (t, 4H), 3.48 (t, 2H), 2.91 (s, 2H), 2.33-2.24 (m, 4H), 1.88-1.23 (m, 68H), 0.85 (t, 12H); CIMS m/z 854.7 [M+H]⁺.

### Synthesis of (1-(4-hydroxybutyl) pyrrolidine-3,4-diyl) bis(butane-4,1-diyl) bis(2-hexyldecanoate) (Compound CY43)

To a solution of compound L19-10 (125 mg, 0.14 mmol) in ethyl acetate (3 mL), was added 10% P(OH)₂/C (50 mg). The reaction mixture was stirred under hydrogen balloon at room temperature for 6 h. The mixture was filtered through a pad of Celite, the filtrates were concentrated, and the crude was purified by column chromatography (12 g SiO₂: 0 to 10% methanol in dichloromethane gradient) to obtain compound CY43 as colorless oil (43 mg, 38%). ¹H-NMR (300 MHz, CDCl₃) δ 4.05 (t, 4H), 3.64 (t, 2H), 3.30 (s, 1H), 2.91 (s, 2H), 2.33-2.24 (m, 4H), 1.87-1.23 (m, 68H), 0.85 (t, 12H); CIMS m/z 864.7 [M+H]⁺. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.7 min, purity: 97.66%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=14.0 min, purity: 88.54%.

### Synthesis of Compound CY61 (1-(4-Hydroxybutyl)piperidine-4,4-diyl)bis(ethane-2,1-diyl) bis(2-hexyldecanoate)

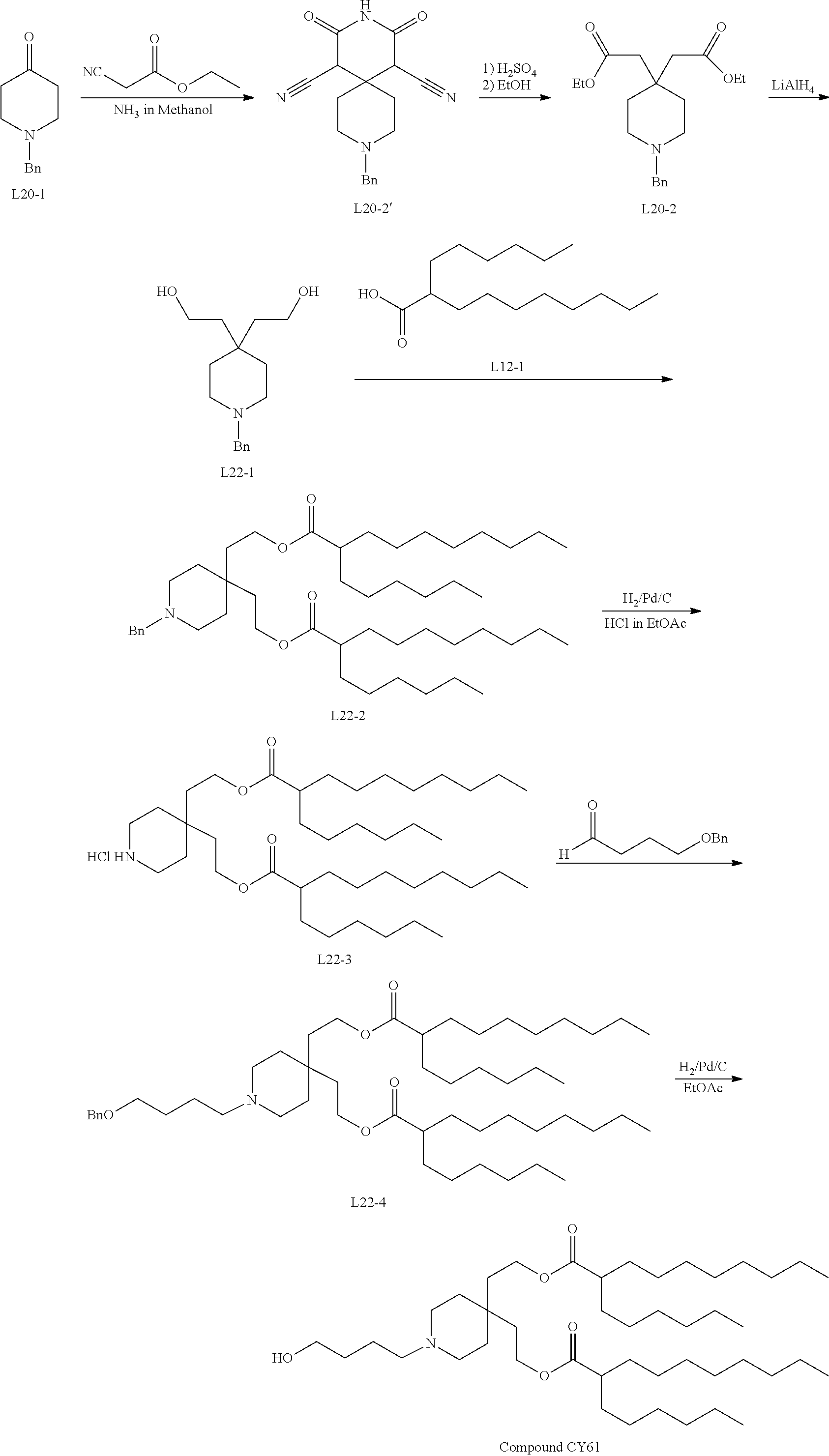

### Synthesis of 9-benzyl-2,4-dioxo-3,9-diazaspiro[5.5]undecane-1,5-dicarbonitrile (L202′)

To ice-cooled 7M ammonia in methanol (120 mL) was added 1-benzyl-4-piperidone (40 g, 212 mmol) followed by ethyl cyanoacetate (45 mL, 2 mmol). The resulted mixture was allowed to stand in refrigerator at −2° C. for five days. The precipitates were filtered and washed with cold methanol. Oven drying overnight provided L20-2′ as off-white solid (23 g, 30%); CIMS m/z [M+H]⁺ 323.

### Synthesis of diethyl 2,2′-(1-benzylpiperidine-4,4-diyl)diacetate (L20-2)

A mixture of L20-2′ (5.0 g, 1.6 mmol), water (5.1 mL) and conc. sulfuric acid (6 mL) was heated at 100° C. for 48 hours. After cooled to room temperature, ethanol (60 mL) was added to the mixture and it was concentrated. The procedure was repeated four times.

Ethanol (40 mL) was then added to the crude product and the solution was heated under reflux for 3 days. After ice-cooling, Na₂CO₃ (6 g) and water were added, and the mixture was concentrated. Ethyl acetate was added and the solution was washed with water and brine and dried over anhydrous Na₂SO₄. Filtration and concentration provided crude material which was purified by flash chromatography (SiO₂: ethyl acetate/hexane 0-100% with 1% triethylamine in the eluent) to yield L20-2 as light-yellow oil (1.85 g, 82%). ¹H-NMR (300 MHz, CDCl₃) δ 7.32-7.21 (m, 5H), 4.11 (q, J=6.5 Hz, 4H), 3.50 (s, 2H), 2.56 (s, 4H), 2.43 (t, J=5.5 Hz, 4H), 1.68 (t, J=6.8 Hz, 4H), 1.24 (t, J=7.1 Hz, 6H); CIMS m/z [M+H]⁺ 348.

### Synthesis of 2,2′-(1-benzylpiperidine-4,4-diyl)bis(ethan-1-ol) (L22-1)

To an ice-cooled solution of 2.0 M lithium aluminum hydride in THF (5.0 mL, 10 mmol) was added slowly a solution of L20-2 (1.85 g, 5.3 mmol) in anhydrous THF (25 mL) under nitrogen atmosphere. The resulting mixture was stirred at room temperature overnight. With ice-water bath cooling, water (0.38 mL), 15% aqueous sodium hydroxide solution (0.38 mL) and water (1.15 mL) were added successively. Filtration through Celite and concentration to yield L22-1 as an oil which slowly solidified to an off-white solid (1.32 g, 94%). ¹H-NMR (300 MHz, CDCl₃) δ 7.39-7.18 (m, 5H), 3.74 (t, J=6.5 Hz, 4H), 3.49 (s, 2H), 2.40 (t, J=5.2 Hz, 4H), 1.67 (t, J=6.8 Hz, 4H), 1.50 (t, J=7.1 Hz, 6H); CIMS m/z [MH⁺] 264.

### Synthesis of (1-benzylpiperidine-4,4-diyl)bis(ethane-2,1-diyl) bis(2-hexyldecanoate) (L22-2)

To a solution of L22-1 (1.32 g, 5 mmol) in DCM (50 mL) was added L12-1 (3.4 g, 13 mmol) followed by DMAP (0.61 g, 5 mmol) and EDC (3.7 g, 20 mmol). The resulting mixture was stirred at room temperature under nitrogen atmosphere for 48 h. The reaction mixture was diluted with DCM (50 mL) and washed with saturated NaHCO₃ aqueous solution (50 mL), water (25 mL) and brine (25 mL). The organic phase was dried over anhydrous Na₂SO₄. Filtration and concentration provided crude material which was purified by flash column chromatography (SiO₂: ethyl acetate/hexane 0-100% with 1% triethylamine in the eluent) to yield L22-2 as an oil which slowly solidified to a white solid (2.6 g, 70%). ¹H-NMR (300 MHz, CDCl₃) δ 7.31-7.19 (m, 5H), 4.12 (q, J=7.1 Hz, 4H), 3.49 (s, 2H), 2.49-2.22 (m, 6H), 1.73-1.12 (m, 56H), 0.87 (t, J=6.3 Hz, 12H); CIMS m/z [M+H]⁺ 740.

### Synthesis of piperidine-4,4-diylbis(ethane-2,1-diyl) bis(2-hexyldecanoate) (L22-3)

To a solution of L22-2 (2.6 g, 3.5 mmol) in 2-propanol (60 mL) was added 10% Pd/C (1.5 g) and 1M HCl in EtOAc (10 mL). The resulting mixture was stirred under a hydrogen balloon and heated in oil bath at 80° C. for 20 h. The reaction mixture was filtered through Celite. The Celite was rinsed with 2-propanol, dichloromethane and EtOAc. The combined filtrate was evaporated to give L22-3 as a light-yellow oil (2.2 g, 95%); 4.20-4.00 (m, 4H), 3.49-2.90 (m, 4H), 2.35-2.15 (m, 2H), 1.95-0.90 (m, 56H), 0.87 (t, J=6.6 Hz, 12H); CIMS m/z [M+H]⁺ 650.

### Synthesis of (1-(4-(benzyloxy)butyl)piperidine-4,4-diyl)bis(ethane-2,1-diyl) bis(2-hexyldecanoate) (L22-4)

To a solution of L22-3 (1.5 g, 2.3 mmol) and 4-benzyloxybutanal (0.8 g, 4.6 mmol) in dichloroethane (60 mL) was added sodium triacetoxyborohydride (1.5 g, 6.9 mmol) followed by acetic acid (0.16 mL, 2.3 mmol). The resulting mixture was stirred at room temperature under nitrogen atmosphere for two days. The reaction mixture was diluted with DCM (40 mL) and washed with saturated NaHCO₃ aqueous solution (50 mL), water (25 mL) and brine (25 mL). The organic phase was dried over anhydrous Na₂SO₄. Filtration and concentration provided crude material which was purified by flash column chromatography (SiO₂: 0 to 100% ethyl acetate in hexane gradient) to yield 122-4 as slightly yellow oil (0.9 g, 48%). ¹H-NMR (300 MHz, CDCl₃) δ 7.35-7.21 (m, 5H), 4.49 (s, 2H), 4.13 (q, J=7.1 Hz, 4H), 3.47 (t, J=5.7 Hz, 211), 2.49-2.20 (m, 811), 1.75-1.12 (m, 60H), 0.87 (t, J=6.0 Hz, 12H); CIMS m/z [M+H]⁺ 812.

### Synthesis of (1-(4-hydroxybutyl)piperidine-4,4-diyl)bis(ethane-2,1-diyl) bis(2-hexyldecanoate) (Compound CY61)

To a solution of L22-4 (0.9 g, 2.1 mmol) in EtOAc (40 mL) was added 10% Pd/C (0.5 g) and 1M HCl in EtOAc (8 mL). The resulting mixture was stirred under a hydrogen balloon overnight. It was then filtered through Celite. The Celite was rinsed with EtOAc (25 mL×3). Concentration provided crude material which was purified by flash column chromatography (SiO₂: ethyl acetate/hexane 0-100% with 1% triethylamine in the eluent) to yield Compound CY61 as a light-yellow oil (130 mg, 16%). ¹H-NMR (300 MHz, CDCl₃) δ 4.12 (q, J=7.1 Hz, 4H), 3.55 (m, 2H), 2.55-2.20 (m, 8H), 1.75-1.12 (m, 60H), 0.87 (t, J=6.3 Hz, 12H); MS (CI): m/z [M+H]⁺ 722.6; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.2 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=12.1 min, purity: 99.21%. The acetylated product CY62 (550 mg) was also isolated as slightly yellow oil. ¹H NMR (300 MHz, CDCl₃): d ppm 4.13 (q, J=7.1 Hz, 4H), 4.06 (q, J=6.3 Hz, 2H), 2.46-2.21 (m, 8H), 2.03 (s, 3H), 1.72-1.15 (m, 60H), 0.87 (t, J=6.3 Hz, 12H); MS (CI): m/z [M+H]⁺ 764.6; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.3 min, purity: 99.83%; UPLC column: Thermo Scientific Hypersil GOLD C4, mobile phase A: acetonitrile with 0.1% trifluoroacetic acid, mobile phase B: water with 0.1% trifluoroacetic acid, use gradient A in B 60% to 100% in 15 min, flow rate: 0.5 mL/min, column temperature: 20±2° C., detector: CAD, t_{R}=13.7 min, purity: 97.57%.

### Synthesis of Compound CY57 (1-(4-hydroxybutyl)piperidine-4,4-diyl)bis(ethane-2,1-diyl) bis(2-hexyldecanoate)

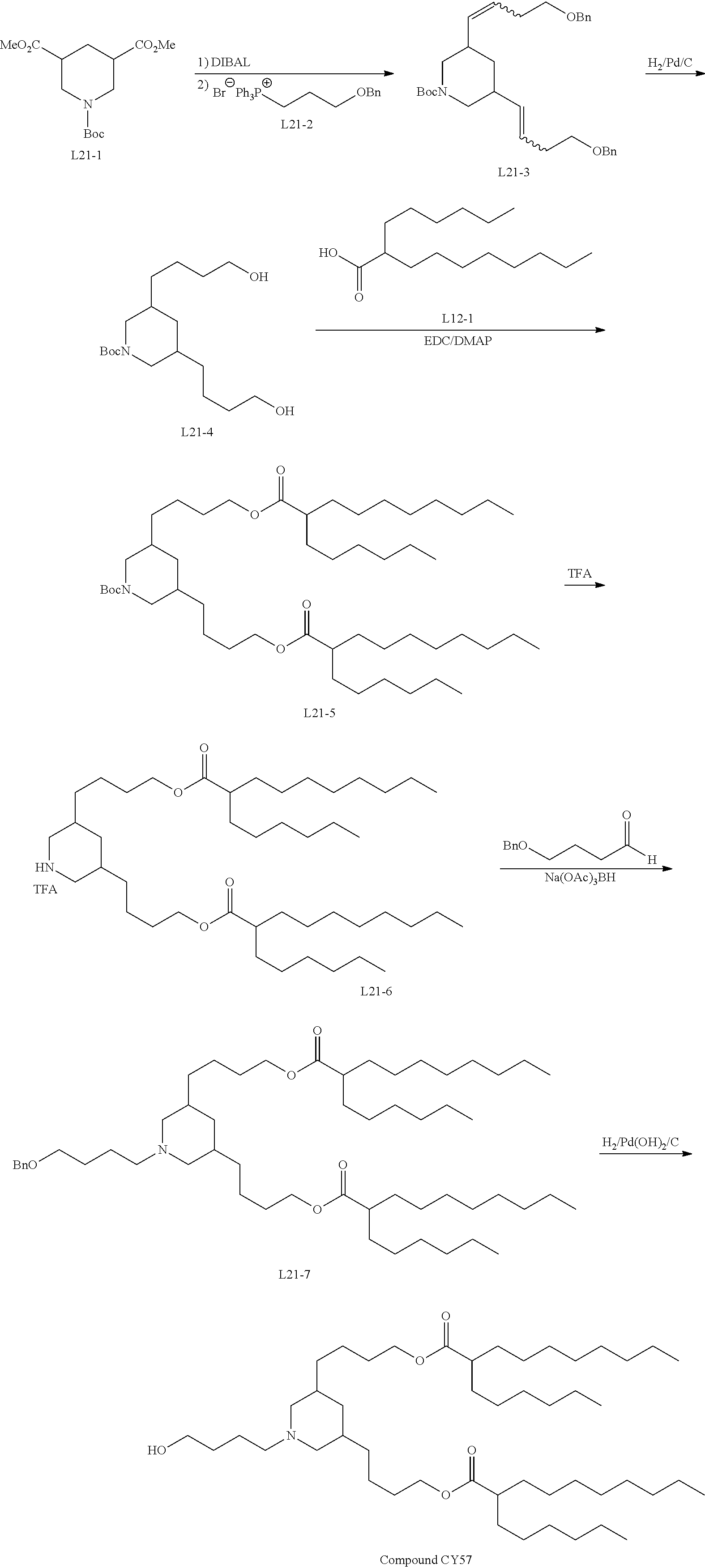

### Synthesis of tert-butyl 3,5-bis(4-(benzyloxy)but-1-en-1-yl)piperidine-1-carboxylate (L21-3)

To a dry ice-acetone bath cooled solution of L21-1 (500 mg, 1.6 mmol) in anhydrous toluene (8 mL) was added 1.0 M diisobutylaluminum hydride in toluene (3.4 mL, 3.4 mmol) under nitrogen atmosphere. The resulted mixture was stirred at −72° C. for 2h. About half of a pre-cooled (−72° C.) solution of benzyloxypropylidene triphenylphosphorane (“Wittig Reagent”, obtained by adding potassium tert-butoxide (1.1 g, 9.3 mmol) to a solution of (3-benzyloxypropyl)triphenyl phosphonium bromide L21-2 (4.86 g, 9.6 mmol) in anhydrous toluene (8 mL) at 0° C.) was stirred at room temperature for 2 h. The reaction mixture was warmed to room temperature and stirred for 16 h. The rest of the solution of Wittig reagent was added, and the reaction was stirred at room temperature for another 16 h. The reaction was then quenched by adding water (15 mL) and extracted with ethyl acetate (25 mL×3). Combined organic extracts were washed with water (25 mL×3) and dried over anhydrous Na₂SO₄. Filtration and concentration provided crude material which was purified by flash column chromatography (SiO₂: ethyl acetate/hexane 0-100) to yield L21-3 as colorless oil (250 mg, 30%). ¹H-NMR (300 MHz, CDCl₃) δ 7.39-7.18 (m, 10H), 5.55-5.40 (m, 2H), 5.17 (t, J=9.1 Hz, 2H), 4.51 (s, 4H), 3.99 (s, br, 4H), 3.49 (t, J=6.9 Hz, 4H), 2.58-2.23 (m, 6H), 1.77-1.68 (m, 1H), 1.45 (s, 9H), 1.09-0.96 (m, 1H); CIMS m/z [M-Boc+H]+405.7.

### Synthesis of tert-butyl 3,5-bis(4-hydroxybutyl)piperidine-1-carboxylate (L21-4)

A mixture of L21-3 (470 mg, 0.9 mmol) and 10% Pd/C (100 mg) in methanol (12 mL) was stirred under a hydrogen balloon at room temperature for 20 h. The reaction mixture was filtered through Celite. The Celite was washed with methanol. The combined filtrate was evaporated to give L21-4 as a light yellow oil (300 mg, 98%); 4.20-3.95 (m, 4H), 3.63 (t, J=6.3 Hz, 4H), 2.25-2.05 (m, 2H), 1.93-1.82 (m, 1H), 1.70-1.05 (m, 23H), 0.69-0.53 (m, 1H); CIMS m/z [M-Boc+H]⁺ 230.

### Synthesis of (1-(tert-butoxycarbonyl)piperidine-3,5-diyl)bis(butane-4,1-diyl) bis(2-hexyldecanoate) (L21-5)

To a solution of L21-4 (300 mg, 0.9 mmol) in DCM (10 mL) was added L12-1 (580 mg, 2.3 mmol) followed by DMAP (110 mg, 0.9 mmol) and EDC (700 mg, 3.6 mmol). The resulting mixture was stirred at room temperature under nitrogen atmosphere for 20 h. The reaction mixture was diluted with DCM (15 mL) and washed with brine (10 mL). The organic phase was dried over anhydrous Na₂SO₄. Filtration and concentration provided crude material which was purified by flash column chromatography (SiO₂: ethyl acetate/hexane 0-100%) to yield L21-5 as colorless oil (600 mg, 82%). ¹H-NMR (300 MHz, CDCl₃) δ 4.20-3.95 (m, 4H), 4.05 (t, J=6.6 Hz, 4H), 2.39-2.22 (m, 2H), 2.21-2.05 (m, 2H), 1.91-1.80 (m, 1H), 1.68-1.11 (m, 69H), 0.86 (t, J=6.3 Hz, 12H) 0.69-0.53 (m, 1H); CIMS m/z [M-Boc+H]⁺ 706.7.

### Synthesis of piperidine-3,5-diylbis(butane-4,1-diyl) bis(2-hexyldecanoate) (L21-6)

To a solution of L21-5 (450 mg, 0.56 mmol) in dichloromethane (3 mL) was added TFA (3 mL) at 0° C. and the reaction mixture was stirred at room temperature for 4 h. The volatile components were removed under reduced pressure and the crude L21-6 (450 mg) was used for the next step without further purification. ¹H-NMR (300 MHz, CDCl₃) δ 4.05 (t, J=6.3 Hz, 4H), 3.49-2.80 (m, 4H), 2.51-2.22 (m, 4H), 2.02-1.01 (m, 61H), 0.69-0.53 (m, 13H); CIMS m/z [M+H]⁺ 706.7.

### Synthesis of (1-(4-(benzyloxy)butyl)piperidine-4,4-diyl)bis(ethane-2,I-diyl) bis(2-hexyldecanoate) (L21-7)

To a solution of L21-6 (450 mg, 0.55 mmol) and 4-benzyloxybutanal (198 mg, 1.1 mmol) in 1,2-dichloroethane (15 mL) was added sodium triacetoxyborohydride (354 mg, 1.6 mmol) followed by acetic acid (36 μL, 0.55 mmol). The resulted mixture was stirred at room temperature under nitrogen atmosphere for 20 h. The reaction mixture was diluted with DCM (20 mL) and sat. aq. sodium bicarbonate solution was slowly added until no bubbles produced. The resulted two phases were separated and the aqueous phase was extracted with DCM (20×2 mL). Combined organic extracts were dried over anhydrous Na₂SO₄. Filtration and concentration provided crude material which was purified by flash column chromatography (SiO₂: ethyl acetate/hexane 0-100% with 1% triethylamine in the eluent) to yield L21-7 as slightly yellow oil (340 mg, 71%). ¹H-NMR (300 MHz, CDCl₃) δ 7.35-7.21 (m, 5H), 4.49 (s, 2H), 4.05 (t, J=6.3 Hz, 4H), 3.47 (m, 2H), 2.95-2.92 (m, 2H), 2.50-2.10 (m, 6H), 1.81-1.70 (m, 1H), 1.65-1.15 (m, 66H), 0.86 (t, J=6.9 Hz, 12H), 0.55-0.42 (m, IH); CIMS m/z [M+H]⁺ 868.

### Synthesis of (1-(4-hydroxybutyl)piperidine-4,4-diyl)bis(ethane-2,1-diyl) bis(2-hexyldecanoate) (Compound CY57)

A mixture of L21-7 (340 mg, 0.4 mmol) and 10% Pd(OH)₂/C (120 mg) in EtOAc (12 mL) was stirred under a hydrogen balloon for 70h. It was then filtered through Celite. The Celite was rinsed with EtOAc (10 mL×3). Concentration provided crude material which was purified by flash column chromatography (SiO₂: ethyl acetate/hexane 0-100% with 1% triethylamine in the eluent) to yield Compound CY57 as a light-yellow oil (171 mg, 56%). ¹H-NMR (300 MHz, CDCl₃) δ 4.04 (t, J=6.3 Hz, 4H), 3.54 (m, 2H), 2.95-2.92 (m, 2H), 2.61-2.22 (m, 6H), 1.85-1.75 (m, IH), 1.76-1.12 (m, 66H), 0.87 (t, J=6.9 Hz, 12H), 0.55-0.42 (m, 1H); MS (CI): m/z [M+H]⁺ 778.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.6 min, purity: >99%; UPLC column: Thermo Scientific Hypersil GOLD C4, mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=13.3 min, purity: 97.05%.

### Synthetic Scheme for CY63

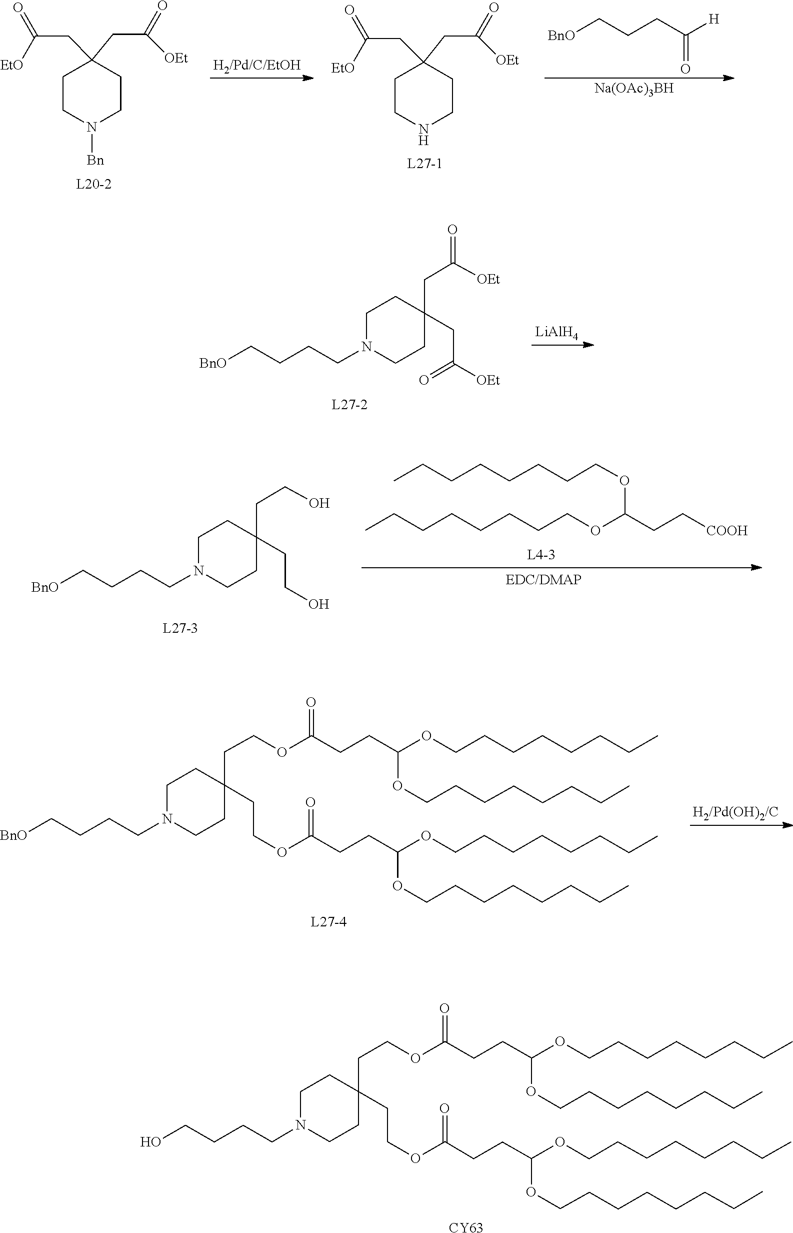

### Synthesis of diethyl 2,2′-(piperidine-4,4-diyl)diacetate (L27-1)

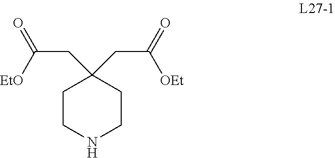 [SMILES: CCOC(=O)CC1(CC(=O)OCC)CCNCC1]

A solution of L20-2 (5.4 g, 15.4 mmol) in ethanol (107 ml) at room temperature was treated with 10% Pd/C (1.1 g) under nitrogen atmosphere. The reaction mixture was evacuated and flushed with H2 gas (3×) and then stirred vigorously under an atmosphere of H₂ (1 atm, H₂-balloon) at room temperature. After 24 h, the reaction mixture was filtered through Celite and the filtrate was concentrated in vacuo to give the crude product, L27-1 (4 g) which was used for the next step without further purification. APCI MS m/z [M+H]⁺ 257.16.

### Synthesis of diethyl 2,2′-(1-(4-(benzyloxy)butyl)piperidine-4,4-diyl)diacetate (L27-2

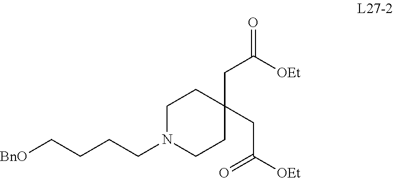 [SMILES: CCOC(=O)CC1(CC(=O)OCC)CCN(CCCCOCc2ccccc2)CC1]

To a mixture of L27-1 (4 g, 15.5 mmol) and 4-(benzyloxy)butanal (5.5 g, 31.1 mmol) in 1,2-dichloroethane (180 mL) was added Na(OAc)₃BH (9.9 g, 46.6 mmol) and acetic acid (1 mL). The reaction mixture was subjected to vacuum/N₂ cycle (3×) and stirred at room temperature for 18 h. The reaction was quenched by slow addition of saturated NaHCO₃ (100 mL) at 0° C. The aqueous phase was extracted using ethyl acetate (100 mL, 3×) and the combined organic phases were dried over anhydrous Na₂SO₄. Filtration followed by concentration provided crude material, which was dissolved in DCM. Silica gel (40 g) and triethyl amine (40 mL) were added to the crude material and shaken for 10-15 min and the solvent was removed under vacuum. The residue was loaded on to an empty flash cartridge, which was then attached to flash purification system loaded with 80 g flash silica column and was purified by flash chromatography (SiO₂: 0 to 10% ethyl acetate in hexane (10% triethylamine)) to yield ethyl L27-2 as slightly yellow oil (3.7 g, 57%). ¹H-NMR (300 MHz, CDCl₃) δ 7.31-7.30 (m, 5H), 4.46 (s, 2H), 4.09-4.04 (m, 4H), 3.47-3.43 (m, 2H), 2.52-2.31 (m, 10H), 1.68-1.57 (m, 8H), 1.22 (t, 6H); APCI MS m/z [M+H]⁺ 420.3.

### Synthesis of 2,2′-(1-(4-(benzyloxy)butyl)piperidine-4,4-diyl)bis(ethan-1-ol) (L27-3)

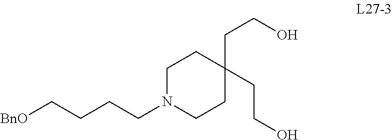 [SMILES: OCCC1(CCO)CCN(CCCCOCc2ccccc2)CC1]

A solution of L27-2 (0.75 g, 1.78 mmol) in THF (14 mL) was cooled in an ice bath (0° C.) and to this was added 2M LiAlH₄ in THF (3.56 mL, 7.14 mmol), dropwise. The ice bath was removed, and the reaction mixture was stirred for 18 h at room temperature. The mixture was diluted with Et₂O (50 mL), cooled in an ice bath, and carefully quenched with water (10 mL), 20% NaOH (10 mL) and water (30 mL). After stirring for 30 min, the aqueous phase was extracted with 20 mL DCM (3×), then the combined organic phase was dried (Na₂SO₄), filtered and concentrated to give L27-3 (0.54 g, 91% yield) as a white solid. APCI MS m/z [M+H]⁺ 336.3.

### Synthesis of 4,4-bis(nonyloxy)butanoic acid (L4-3(T9))

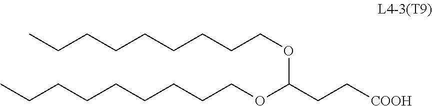 [SMILES: CCCCCCCCCOC(CCC(=O)O)OCCCCCCCCC]

Prepared following Procedure B described in Compound L4L synthesis. Compound L4-3(T9) was isolated as light-yellow oil in a yield of 11.8 g (98%). ¹HNMR (CDCl₃) δ: 4.53-4.56 (t, 1H), 3.57-3.60 (m, 2H), 3.40-3.43 (m, 2H), 2.39-2.41 (t, 2H), 1.90-1.95 (m, 2H), 1.54-1.56 (M, 4H), 1.26 (bs, 28H), 0.85-0.87 (t, 6H); CIMS m/z [M−H]⁻ 371.

### Synthesis of (1-(4-hydroxybutyl)piperidine-4,4-diyl)bis(ethane-2,1-diyl) bis(4,4-bis(octyloxy) butanoate) (L27-4) [Procedure E]

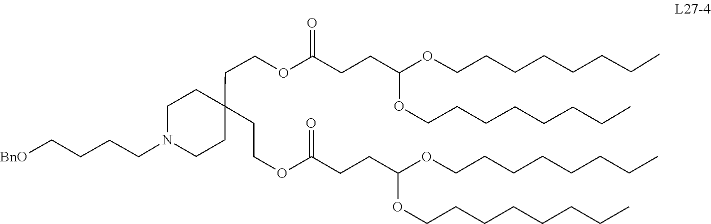 [SMILES: CCCCCCCCOC(CCC(=O)OCCC1(CCOC(=O)CCC(OCCCCCCCC)OCCCCCCCC)CCN(CCCCOCc2ccccc2)CC1)OCCCCCCCC]

To a 250 mL round bottom flask containing L4-3(T9) (1 g, 2.9 mmol, 2.5 eq), EDC (1.01 g, 5.28 mmol, 4 eq), DMAP (161 mg, 1.32 mmol, 1 eq) and L27-3 (440 mg, 1.32 mmol, 1 eq) was added anhydrous dichloromethane (20 mL) and the reaction mixture was stirred at room temperature overnight. After completion of the reaction about 30g of flash silica was added and the contents were stirred well to get a uniform mixture. Solvent was removed from this mixture under vacuum. The residue was loaded on to an empty flash cartridge, which was then attached to a flash purification system loaded with flash silica column and was purified by flash chromatography (SiO₂: hexane (10% triethyl amine)/ethyl acetate 0-20%) to get Compound L27-4 (0.94 g, 73%) as slightly yellow oil. ¹H-NMR (300 MHz, CDCl₃) δ 7.33-7.31 (m, 5H), 4.48-4.47 (m, 4H), 4.10-4.08 (m, 4H), 3.56-3.37 (m, 10H), 2.37-2.32 (m, 10H), 1.90-1.80 (m, 20H), 1.31-1.10 (m, 40H), 0.84 (t, 12H); APCI MS m/z [M+H]⁺ 988.8.

### Synthesis of (1-(4-hydroxybutyl)piperidine-4,4-diyl)bis(ethane-2,1-diyl) bis(4,4-bis(octyloxy) butanoate) (CY63) [Procedure F]

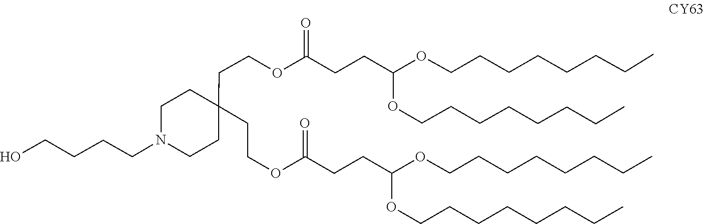 [SMILES: CCCCCCCCOC(CCC(=O)OCCC1(CCOC(=O)CCC(OCCCCCCCC)OCCCCCCCC)CCN(CCCCO)CC1)OCCCCCCCC]

To a 250 mL round bottom flask containing L27-4 (560 mg, 0.56 mmol) and 10% Pd/C (186 mg) was added ethyl acetate (20 mL) and then the reaction mixture was subjected to vacuum/N₂ cycle (3×) followed by another cycle of vacuum/H2 (3×). The reaction mixture was placed under 1 atm H2 (hydrogen balloon) and left to stir overnight. The reaction mixture was diluted with ethyl acetate (100 mL) and then filtered through Celite, washed with ethyl acetate, and then the solvent was removed under vacuum to dryness to give the crude product as a light brown oil CY63 (132 mg, 26%). ¹H-NMR (300 MHz, CDCl₃) δ 4.48 (t, 4H), 4.12-4.07 (m, 4H), 3.55-3.39 (m, 10H), 2.46-2.31 (m, 10H), 1.90-1.88 (m, 4H), 1.66-1.51 (m, 20H), 1.30-1.00 (m, 40H), 0.86 (t, 12H); APCI MS m/z [M+H]⁺ 898.8; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.6 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.8 min, purity: >99%.

### Synthetic Scheme for CY69

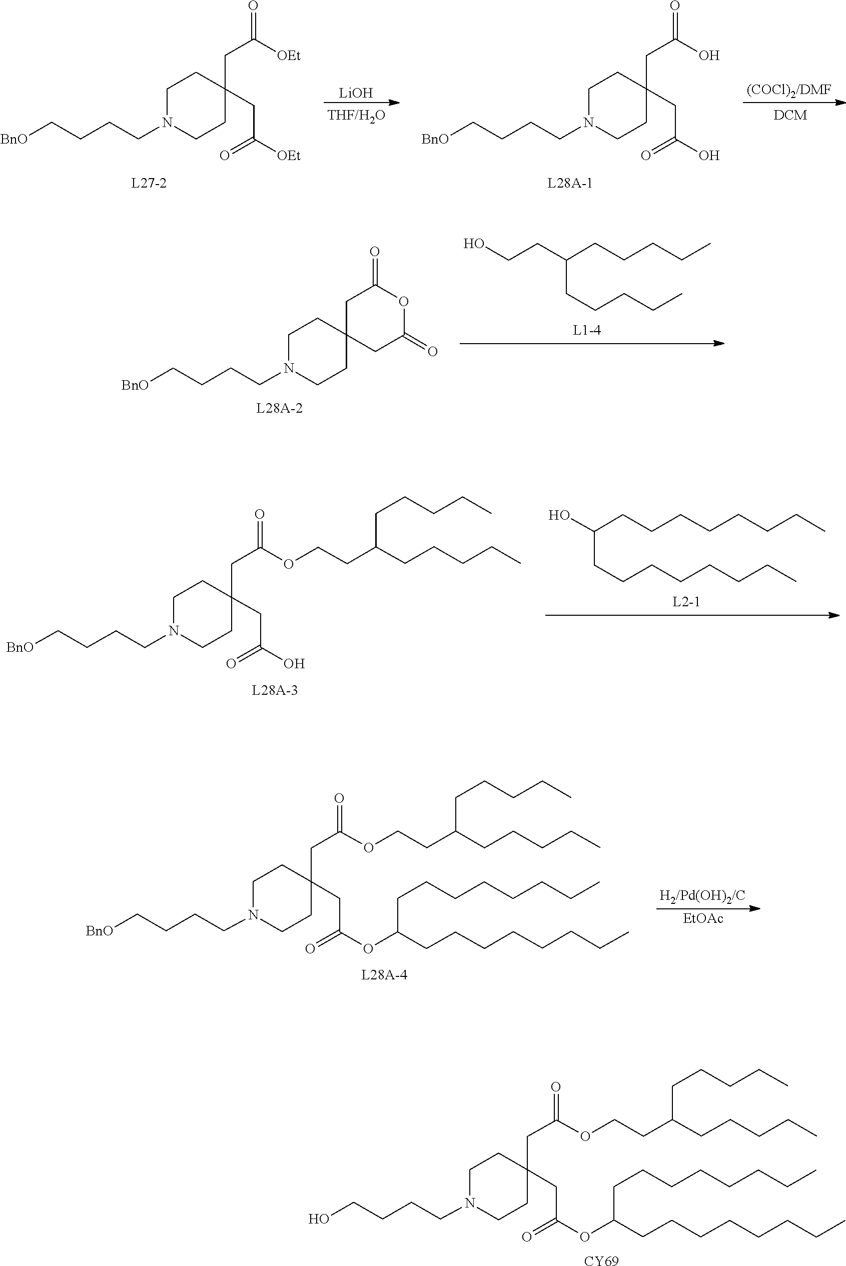

### Synthesis of 2,2′-(1-(4-(benzyloxy)butyl)piperidine-4,4-diyl)diacetic acid (L28A-1)

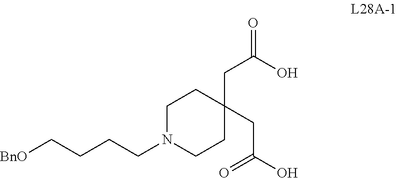 [SMILES: O=C(O)CC1(CC(=O)O)CCN(CCCCOCc2ccccc2)CC1]

To a solution of diester L27-2 (0.9 g, 2.1 mmol) in THF (15 mL) and methanol (2.5 mL) was added a solution of LiOH (0.36 g, 6.4 mmol) in water (5 mL). The mixture was stirred at room temperature for 20 h. While cooling in ice-water bath, the reaction mixture pH was adjusted to 4. Volatile components were removed under reduced pressure and the residue was lyophilized to give an off-white solid which was purified by reverse column chromatography (acetonitrile/water 0-100) to yield L28A-1 as off-white foam solid (0.62 g, 80%). ¹H-NMR (300 MHz, CDCl₃) δ 7.39-7.18 (m, 5H), 4.47 (s, 2H), 3.48 (t, J=6.3 Hz, 2H), 2.90-2.56 (m, 6H), 2.43 (m, 4H), 2.05-1.56 (m, 8H); CIMS m/z [M−H]⁻ 361.5.

### Synthesis of 9-(4-(benzyloxy)butyl)-3-oxa-9-azaspiro[5.5]undecane-2,4-dione (L28A-2)

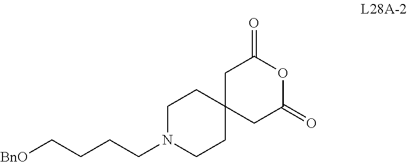 [SMILES: O=C1CC2(CCN(CCCCOCc3ccccc3)CC2)CC(=O)O1]

To a solution of L28A-1 (0.5 g, 1.4 mmol) in anhydrous DCM (15 mL) and pyridine (2 mL) at 0° C. under nitrogen atmosphere was added anhydrous DMF (1 drop) and oxalyl chloride (0.15 mL, 4.2 mmol). After completion of the addition, the mixture was stirred at room temperature for 18 h. More oxalyl chloride (0.15 mL, 4.2 mmol) was added and the mixture was stirred at room temperature for 20 h. The reaction mixture was concentrated and co-evaporated with anhydrous toluene to give L28A-2 as a light yellow oil (0.48 g, 99%); CIMS m/z [M+H]⁺ 346.2.

### Synthesis of 2-(1-(4-(benzyloxy)butyl)-4-(2-oxo-2-((3-pentyloctyl)oxy)ethyl)piperidin-4-yl)acetic acid (L28A-3)

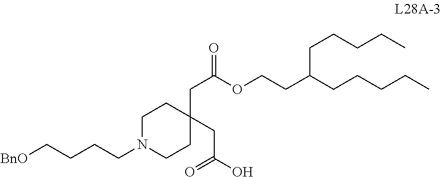 [SMILES: CCCCCC(CCCCC)CCOC(=O)CC1(CC(=O)O)CCN(CCCCOCc2ccccc2)CC1]

To a solution of L28A-2 (480 mg, 1.4 mmol) in DCM (15 mL) and pyridine (2 mL) at 0° C. was added L1-4 (800 mg, 4.0 mmol). The resulting mixture was stirred at room temperature under nitrogen atmosphere for 18h. More L1-4 (160 mg, 1.4 mmol) was added, and the mixture was stirred at 50° C. for 20 h. The reaction mixture was concentrated, and the crude material was purified by flash column chromatography (SiO₂: Methanol/DCM 0-30% with 5% triethylamine) to yield L28A-3 as light-yellow solid (330 mg, 44%). ¹H-NMR (300 MHz, CDCl₃) δ 7.41-7.15 (m, 5H), 4.46 (s, 2H), 4.06 (t, J=6.5 Hz, 2H), 2.89-2.36 (m, 10H), 1.97-1.15 (m, 29H), 0.87 (t, J=6.8 Hz, 6H); CIMS m/z [M+H]⁺ 546.4.

### Synthesis of heptadecan-9-yl 2-(1-(4-(benzyloxy)butyl)-4-(2-oxo-2-((3-pentyloctyl)oxy)ethyl) piperidin-4-yl)acetate (L28A-4)

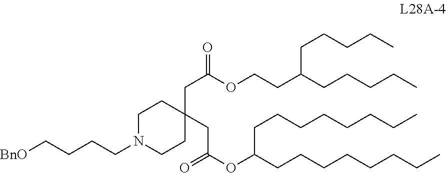 [SMILES: CCCCCCCCC(CCCCCCCC)OC(=O)CC1(CC(=O)OCCC(CCCCC)CCCCC)CCN(CCCCOCc2ccccc2)CC1]

To a solution of L28A-3 (320 mg, 0.58 mmol) in DCM (10 mL) was added heptadecan-9-ol (L2-1) (225 mg, 0.88 mmol) followed by DMAP (38 mg, 0.3 mmol) and EDC (225 mg, 1.2 mmol). The resulting mixture was stirred at room temperature under nitrogen atmosphere for 18 h. The reaction mixture was diluted with DCM (15 mL) and washed with brine (10 mL). The organic phase was dried over anhydrous Na₂SO₄. Filtration and concentration provided crude material which was purified by flash column chromatography (SiO₂: ethyl acetate/hexane 0-100%) to yield L28A-4 as colorless oil (305 mg, 66%). ¹H-NMR (300 MHz, CDCl₃) δ 7.38-7.21 (m, 5H), 4.84 (m, 1H), 4.49 (s, 2H), 4.05 (t, J=7.1 Hz, 2H), 3.47 (t, J=5.9 Hz, 2H), 2.60-2.28 (m, 10H), 1.76-1.15 (m, 55H), 0.87 (t, J=6.0 Hz, 12H); CIMS m/z [M+H]⁺ 784.8.

### Synthesis of heptadecan-9-yl 2-(1-(4-hydroxybutyl)-4-(2-oxo-2-((3-pentyloctyl)oxy)ethyl) piperidin-4-yl)acetate (Compound CY69)

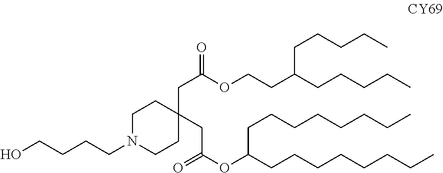 [SMILES: CCCCCCCCC(CCCCCCCC)OC(=O)CC1(CC(=O)OCCC(CCCCC)CCCCC)CCN(CCCCO)CC1]

A mixture of L28A-4 (300 mg, 0.38 mmol) and 10% Pd(OH)₂/C (150 mg) in EtOAc (15 mL) was stirred under a hydrogen balloon for 80 h. The mixture was then filtered through Celite. The Celite was rinsed with EtOAc (10 mL×3). Concentration of the filtrate provided crude material which was purified by flash column chromatography (SiO₂: ethyl acetate/hexane 0-100% with 1% triethylamine in the eluent) to yield Compound CY69 as a light-yellow oil (241 mg, 91%). ¹H-NMR (300 MHz, CDCl₃) δ 4.84 (m, 1H), 4.05 (t, J=7.4 Hz, 2H), 3.56 (m, 2H), 2.71-2.35 (m, 10H), 1.82-1.43 (m, 15H), 1.36-1.15 (m, 40H), 0.87 (t, J=5.2 Hz, 12H); MS (CI): m/z [M+H]⁺ 694.6; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.6 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=13.4 min, purity: >99%.

### Synthetic Scheme for CY65

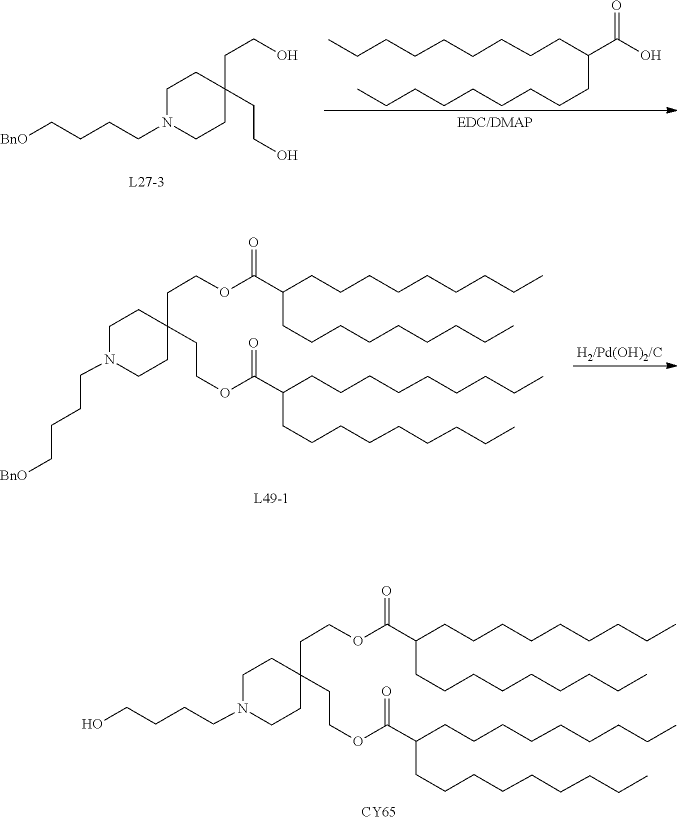 [SMILES: OCCC1(CCO)CCN(CCCCOCc2ccccc2)CC1 ; CCCCCCCCCC(CCCCCCCCC)C(=O)O ; CCCCCCCCCC(CCCCCCCCC)C(=O)OCCC1(CCOC(=O)C(CCCCCCCCC)CCCCCCCCC)CCN(CCCCOCc2ccccc2)CC1 ; "L49-1" ; "CY65" ; CCCCCCCCCC(CCCCCCCCC)C(=O)OCCC1(CCOC(=O)C(CCCCCCCCC)CCCCCCCCC)CCN(CCCCO)CC1]

### Synthesis of (1-(4-(benzyloxy)butyl)piperidine-4,4-diyl)bis(ethane-2,I-diyl) bis(2-nonylundecanoate) (L49-4)

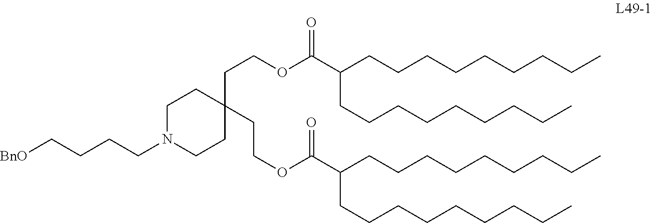 [SMILES: CCCCCCCCCC(CCCCCCCCC)C(=O)OCCC1(CCOC(=O)C(CCCCCCCCC)CCCCCCCCC)CCN(CCCCOCc2ccccc2)CC1]

Prepared following Procedure E described in Compound L27 synthesis. Compound L49-1 was isolated as colorless oil (580 mg, 43%). ¹H-NMR (300 MHz, CDCl₃) δ 7.33-7.26 (m, 5H), 4.48 (s, 2H), 4.12 (t, 4H), 3.46 (t, 2H), 2.45-2.24 (m, 7H), 1.70-1.37 (m, 19H), 1.29-1.15 (m, 58H), 0.86 (t, 12H); CIMS m/z [M+H]⁺ 925.56.

### Synthesis of (1-(4-hydroxybutyl)piperidine-4,4-diyl)bis(ethane-2,1-diyl) bis(2-nonylundecanoate) (CY65)

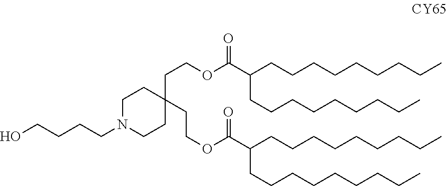 [SMILES: CCCCCCCCCC(CCCCCCCCC)C(=O)OCCC1(CCOC(=O)C(CCCCCCCCC)CCCCCCCCC)CCN(CCCCO)CC1]

Prepared following Procedure F described in Compound CY63 synthesis. Compound CY65 was isolated as colorless oil (0.52 g, 97%). ¹H-NMR (300 MHz, CDCl₃) δ 4.11 (m, 4H), 3.56 (t, 2H), 2.55-241 (m, 4H), 2.27-2.21 (m, 2H), 1.68-1.57 (m, 16H), 1.28-1.15 (m, 54H), 0.86 (t, 12H); CIMS m/z [M+H]⁺ 834.1. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=12.1 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=15.4 min, purity: >98.1%.

### Synthetic Scheme for CY66

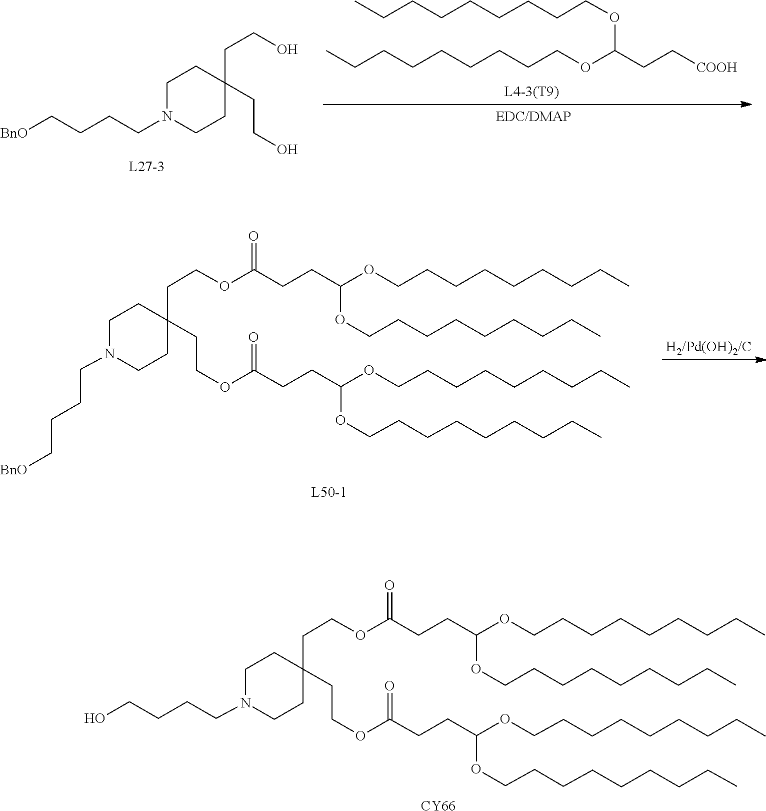

### Synthesis of (1-(4-(benzyloxy)butyl)piperidine-3,5-diyl)bis(ethane-2,1-diyl) bis(4,4-bis (nonyl oxy)butanoate) (L50-1)

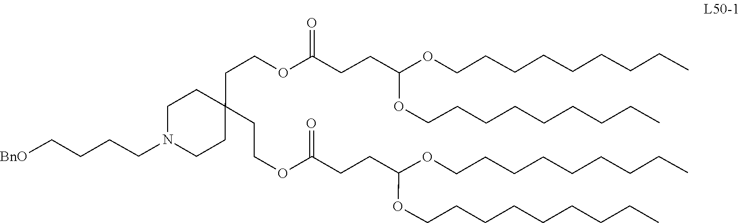 [SMILES: CCCCCCCCCOC(CCC(=O)OCCC1(CCOC(=O)CCC(OCCCCCCCCC)OCCCCCCCCC)CCN(CCCCOCc2ccccc2)CC1)OCCCCCCCCC]

Prepared following Procedure E described in Compound CY63 synthesis. Compound L50-1 (0.55 g, 44%) was isolated as colorless oil. ¹H-NMR (300 MHz, CDCl₃) δ 7.33-7.32 (m, 5H), 4.49-4.47 (m, 4H), 4.12-4.10 (m, 4H), 3.56-3.37 (m, 10H), 2.38-2.33 (m, 10H), 2.04-1.89 (m, 4H), 1.66-1.50 (m, 20H), 1.40-0.99 (m, 48H), 0.87 (t, 12H); APCI-MS: m/z [M+H]⁺ 1045.0.

### Synthesis of (1-(4-hydroxybutyl)piperidine-3,5-diyl)bis(ethane-2,1-diyl) bis(4,4-bis(nonyloxy) butanoate) (CY66)

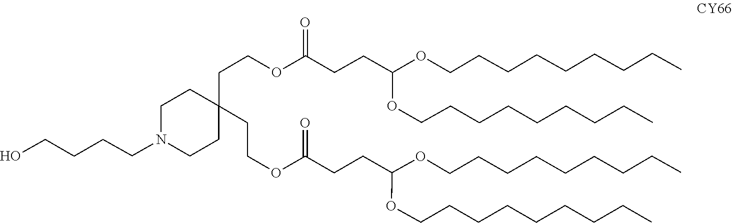 [SMILES: CCCCCCCCCOC(CCC(=O)OCCC1(CCOC(=O)CCC(OCCCCCCCCC)OCCCCCCCCC)CCN(CCCCO)CC1)OCCCCCCCCC]

Prepared following Procedure F described in Compound CY63 synthesis. Compound CY66 (0.2 g, 44%) was isolated as colorless oil. ¹H-NMR (300 MHz, CDCl₃) δ 4.47 (t, 4H), 4.13-4.10 (m, 4H), 3.56-3.40 (m, 10H), 2.57-2.39 (m, 10H), 1.91-1.89 (m, 4H), 1.67-1.52 (m, 26H), 1.37-1.00 (m, 48H), 0.87 (t, 12H); APCI-MS: m/z [M+H]⁺ 954.7; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.6 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.8 min, purity: >99%.

### Synthetic Scheme for CY67

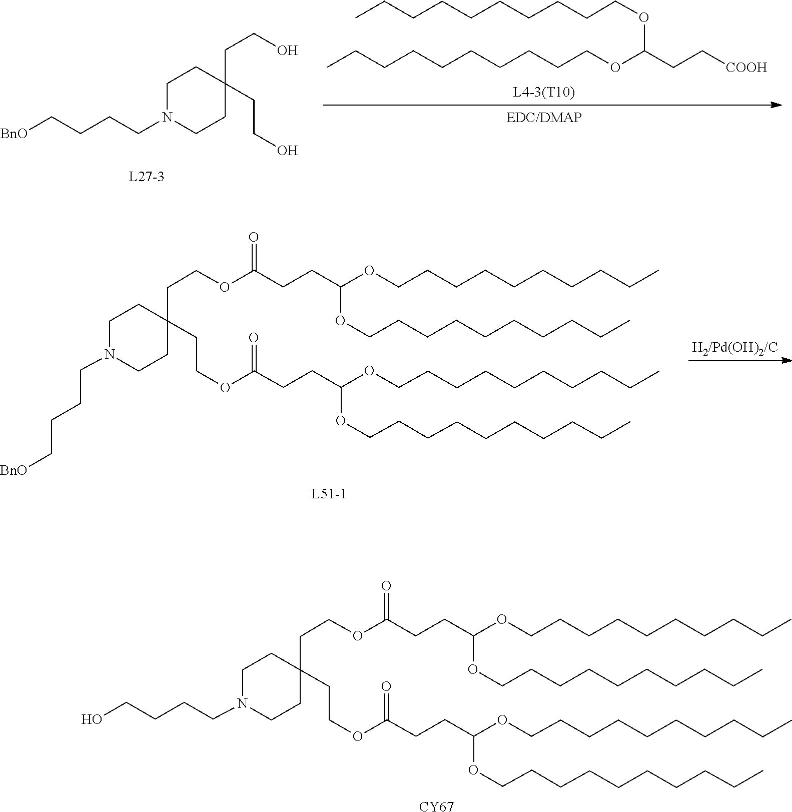 [SMILES: OCCC1(CCO)CCN(CCCCOCc2ccccc2)CC1 ; CCCCCCCCCCOC(CCC(=O)O)OCCCCCCCCCC ; CCCCCCCCCCOC(CCC(=O)OCCC1(CCOC(=O)CCC(OCCCCCCCCCC)OCCCCCCCCCC)CCN(CCCCOCc2ccccc2)CC1)OCCCCCCCCCC ; "L51-1" ; "CY67" ; CCCCCCCCCCOC(CCC(=O)OCCC1(CCOC(=O)CCC(OCCCCCCCCCC)OCCCCCCCCCC)CCN(CCCCO)CC1)OCCCCCCCCCC]

### Synthesis of (1-(4-(benzyloxy)butyl)piperidine-4,4-diyl)bis(ethane-2,1-diyl) bis(4,4-bis(decyloxy)butanoate) (L51-1)

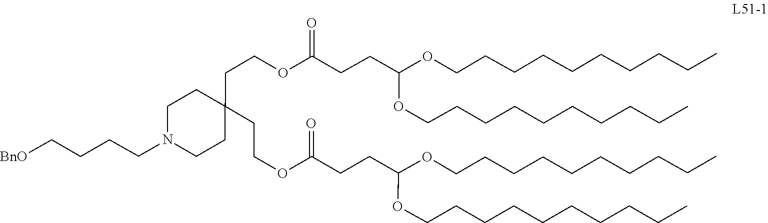 [SMILES: CCCCCCCCCCOC(CCC(=O)OCCC1(CCOC(=O)CCC(OCCCCCCCCCC)OCCCCCCCCCC)CCN(CCCCOCc2ccccc2)CC1)OCCCCCCCCCC]

Prepared following Procedure E described in Compound CY63 synthesis. L51-1 (1.16 g, 88%), colorless oil, H-NMR (300 MHz, CDCl₃) δ 7.33-7.25 (m, 5H), 4.51-4.46 (m, 4H), 4.11 (t, J=7.5 Hz, 4H), 3.62-3.33 (m, 10H), 2.45-2.26 (m, 10H), 1.97-1.85 (m, 4H), 1.73-1.41 (m, 18H), 1.40-1.15 (m, 58H), 0.87 (t, J=6.3 Hz, 12H); MS (CI): m/z [M+H]⁺ 1100.8.

### Synthesis of (1-(4-hydroxybutyl)piperidine-4,4-diyl)bis(ethane-2,I-diyl) bis(4,4-bis(decyloxy)butanoate) (CY67)

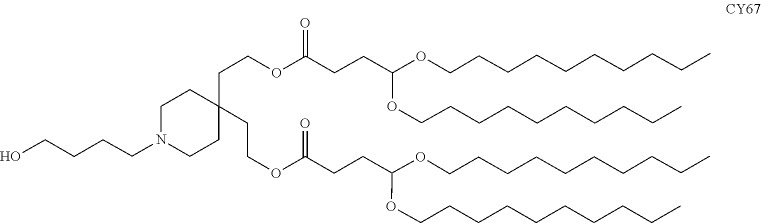 [SMILES: CCCCCCCCCCOC(CCC(=O)OCCC1(CCOC(=O)CCC(OCCCCCCCCCC)OCCCCCCCCCC)CCN(CCCCO)CC1)OCCCCCCCCCC]

Prepared following Procedure F described in Compound CY63 synthesis. Compound CY67 (615 mg, 58%), colorless oil, ¹H-NMR (300 MHz, CDCl₃) δ 4.48 (t, J=5.6 Hz, 2H), 4.11 (t, J=7.4 Hz, 4H), 3.61-3.32 (m, 10H), 2.65-2.30 (m, 10H), 1.98-1.85 (m, 4H), 1.76-1.15 (m, 76H), 0.91-0.80 (m, 12H); MS (CI): m/z [M+H]⁺ 1010.8; Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: mL/min, column temperature: 20±2° C., detector: ELSD, t_{R}=12.4 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=16.0 min, purity: 98%.

### Synthetic Scheme for CY71

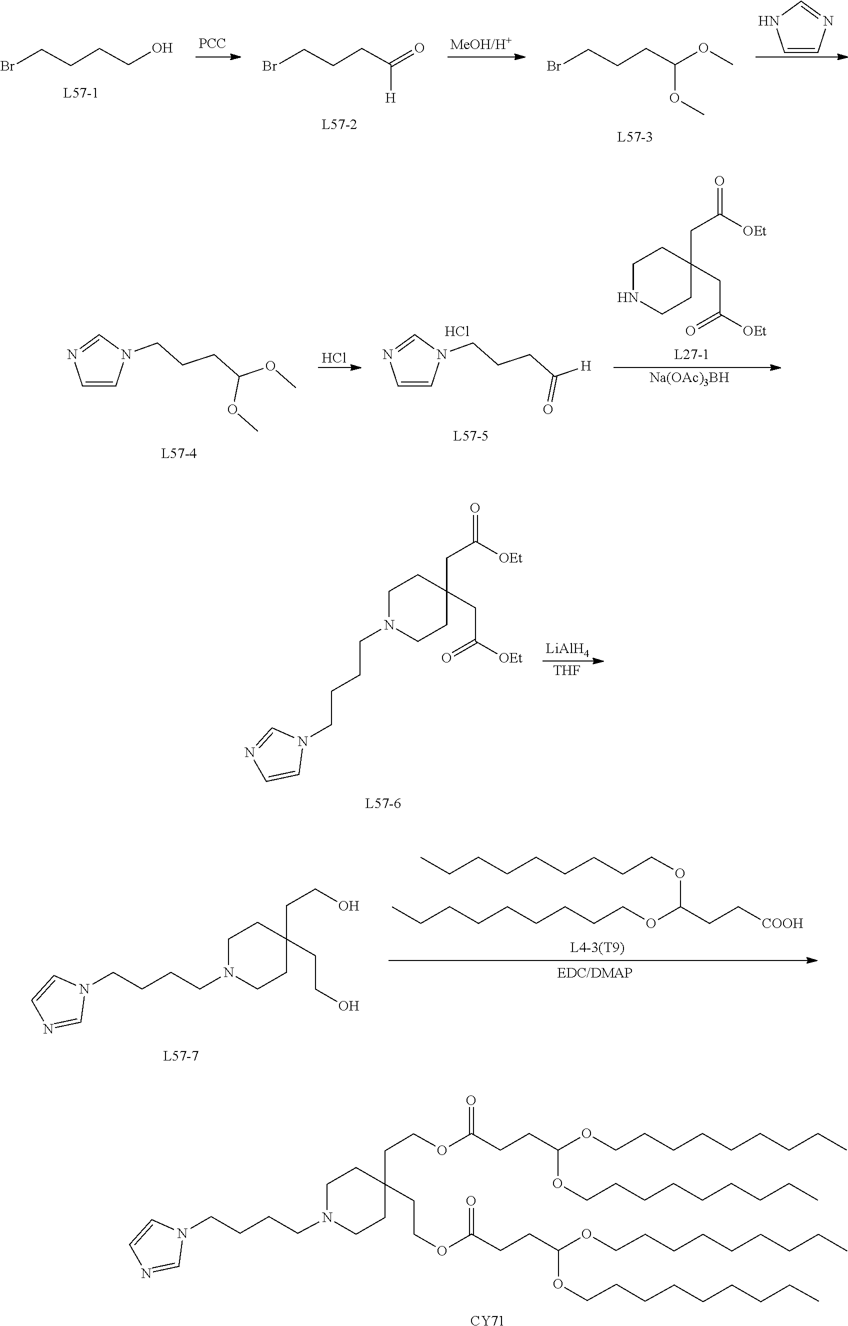

### Synthesis of 4-bromobutanal (L57-2)

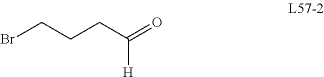

To a solution of pyridinium chlorochromate (PCC) (12.14 g, 56.55 mmol) in DCM (75 mL) was added 4-bromobutan-1-ol (5.77 g, 37.7 mmol) in DCM (25 mL) over 10 min (intermittent cooling was required to prevent solvent reflux). The reaction mixture was stirred at room temperature for 2 h and then diluted with diethyl ether. The upper ether phase was decanted from the flask and filtered through celite and the celite cake was washed with ether. Combined ether phases were evaporated under reduced pressure to get crude 4-bromobutanal L57-2, which was used for the next step without further purification (4.5 g, crude); ¹H-NMR (300 MHz, CDCl₃) δ 9.81 (s, 1H), 3.74 (m, 2H), 2.18 (m, 2H), 1.84 (s, 2H).

### Synthesis of 4-bromo-1,1-dimethoxybutane (L57-3)

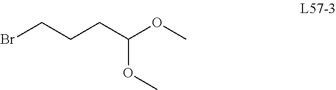

4-bromobutanal L57-2 (4.5 g, crude) was dissolved in methanol (10 mL), then 2N HCl in ether (10 mL) was then added in. The reaction mixture was stirred at room temperature overnight. The volatile components were evaporated under reduced pressure to yield 4-bromo-1,1-dimethoxybutane L57-3 as light-yellow oil (3.9 g, crude); ¹H-NMR (300 MHz, CDCl₃) δ 4.38 (m, 1H), 3.4 (m, 2H), 3.32 (s, 6H), 1.91 (m, 2H), 1.75 (m, 2H).

### Synthesis of 1-(4,4-dimethoxybutyl)-1H-imidazole (L57-4)

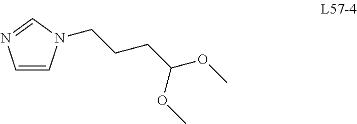

To a solution of imidazole (1.48 g, 21.76 mmol) in anhydrous THF (40 mL) at 5-10° C. was added NaH (948 mg, 23.74 mmol, 60% in mineral oil) portionwise with stirring. The resulting mixture was then stirred at room temperature for 2h. To the suspension was added dropwise 4-bromo-1,1-dimethoxybutane L57-3 (3.9 g, 19.79 mmol) in THF (10 mL) over a period of 15 min and the reaction was further stirred for 3 h at room temperature to achieve a uniform mixture. The reaction mixture was heated at 60° C. overnight, cooled to room temperature and filtered. THF was removed under reduced pressure and the residue was purified by flash chromatography (SiO₂: 0-5% MeOH in DCM gradient) to yield 1-(4,4-dimethoxybutyl)-1H-imidazole L57-4 (850 mg, 12% over 3 steps). ¹H-NMR (300 MHz, CDCl₃) δ 7.45 (s, 1H), 7.04 (s, 1H), 6.9 (s, 1H), 4.32 (t, J=5.49 Hz, 1H), 3.95 (t, J=7.14 Hz, 2H), 3.29 (s, 6H), 1.84 (m, 2H), 1.58 (m, 2H); CIMS m/z [M+H]⁺ 185.

### Synthesis of 1-(4-oxobutyl)-1H-imidazol-1-ium chloride (L57-5)

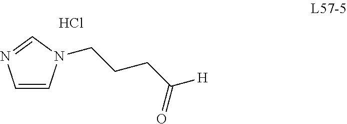

To a solution of 1-(4,4-dimethoxybutyl)-1H-imidazole L57-4 (1.05 g, 5.7 mmol) in THF (5.0 mL), was added 1.5N HCl (5.0 mL). The reaction mixture was stirred at room temperature overnight. THF was evaporated and water layer was washed with DCM (10 mL) and EtOAc (10 mL) to remove impurities. the aqueous layer was evaporated under reduced pressure followed by co-evaporation with acetonitrile (2×10 mL) and toluene (2×10 mL) and dried under high vacuum for 24 h to yield 1-(4-oxobutyl)-1H-imidazol-1-ium chloride L57-5 as a light-yellow gummy solid which was used for the next step without further purification (1.0 g, crude). ¹H-NMR (300 MHz, DMSO-D6) 6 9.63 (s, 1H), 9.21 (s, 1H), 7.81 (s, 1H), 7.7 (s, 1H), 4.19 (m, 2H), 3.34-3.62 (m, 2H) 2.05 (m, 2H); CIMS m/z [M+H]⁺ 139.

### Synthesis of diethyl 2,2′-(1-(4-(1H-imidazol-1-yl)butyl)piperidine-4,4-diyl)diacetate (L57-6)

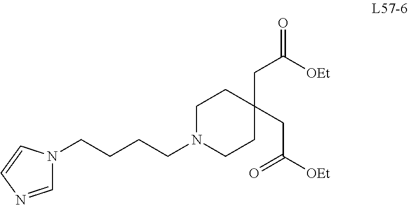 [SMILES: CCOC(=O)CC1(CC(=O)OCC)CCN(CCCCn2ccnc2)CC1]

To a solution of diethyl 2,2′-(piperidine-4,4-diyl)diacetate (850 mg, 3.5 mmol) in a mixture of DMF (5 mL) and DCE (5 mL) was added 1-(4-oxobutyl)-1H-imidazol-1-ium chloride L57-5 (1.0 g, 5.74 mmol) in DMF (5 mL), followed by addition of Na(OAc)₃BH (2.22 g, 10.5 mmol) and AcOH (240 μL, 4.2 mmol). The reaction mixture was stirred at room temperature under nitrogen for 18 hours. LC-MS confirms completion of the reaction. The reaction mixture was diluted with DCM and washed with Sat. NaHCO₃. Aqueous layer was extracted with DCM (3×50 mL). Combined organic layers were dried over Na₂SO₄ and concentrated under reduced pressure to get crude product which was purified by flash chromatography (SiO₂: 0-6% MeOH in DCM gradient) to yield diethyl 2,2′-(1-(4-(1H-imidazol-1-yl)butyl)piperidine-4,4-diyl)diacetate L57-6 (600 mg, 45%). ¹H-NMR (300 MHz, CDCl₃) δ 7.45 (s, 1H), 7.04 (s, 1H), 6.89 (s, 1H), 4.08 (q, J=7.14 Hz, 4H), 3.93 (t, J=7.14 Hz, 2H), 2.53 (s, 4H), 2.36 (m, 6H), 1.78 (m, 2H), 1.67 (m, 4H), 1.5 (m, 2H), 1.23 (t, J=7.14 Hz, 6H); CIMS m/z [M+H]⁺ 380.

### Synthesis of 2,2′-(1-(4-(1H-imidazol-1-yl)butyl)piperidine-4,4-diyl)bis(ethan-1-ol) (L57-7)

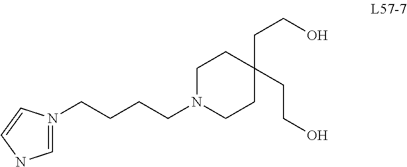

To a solution of 2,2′-(1-(4-(1H-imidazol-1-yl)butyl)piperidine-4,4-diyl)diacetate L57-6 (600 mg, 1.58 mmol) in anhydrous THF (10 mL) and 0° C. was added dropwise a solution of LiAlH₄ in anhydrous THF (2.0 M, 1.6 mL, 3.16 mmol) under nitrogen. The resulting reaction mixture was stirred at room temperature overnight. The reaction mixture was cooled to 0° C. and Na₂SO₄·10H₂O was added slowly until all gas evolution stopped. After filtration through celite, the celite cake was washed with THF. Combined filtrates were concentrated under reduced pressure to give 2,2′-(1-(4-(1H-imidazol-1-yl)butyl)piperidine-4,4-diyl)bis(ethan-1-ol) L57-7 as colorless viscous liquid, which was used for the next step without further purification (440 mg, crude). ¹H-NMR (300 MHz, CDCl₃) δ 7.45 (s, 1H), 7.02 (s, 1H), 6.89 (s, 1H), 3.93 (t, J=7.14 Hz, 2H), 3.7 (t, J=6.6 Hz, 4H), 2.33 (m, 6H), 1.77 (m, 2H), 1.65 (t, J=6.75 Hz, 4H), 1.55 (m, 2H), 1.47 (m, 4H); CIMS m/z [M+H]⁺ 296.

### Synthesis of (1-(4-(1H-imidazol-1-yl)butyl)piperidine-4,4-diyl)bis(ethane-2,1-diyl) bis(4,4-bis(nonyloxy)butanoate) (CY71)

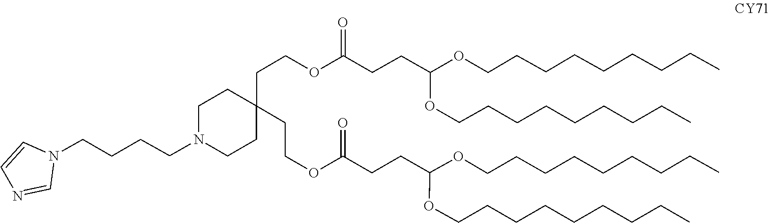 [SMILES: CCCCCCCCCOC(CCC(=O)OCCC1(CCOC(=O)CCC(OCCCCCCCCC)OCCCCCCCCC)CCN(CCCCn2ccnc2)CC1)OCCCCCCCCC]

To a solution of 4,4-bis(nonyloxy)butanoic acid (1.21 g, 3.27 mmol) in DCM (15 mL) was added DMAP (363 mg, 2.98 mmol) and EDC (1.25 g, 6.55 mmol). The reaction mixture was stirred at room temperature for 15 min, 2,2′-(1-(4-(1H-imidazol-1-yl)butyl)piperidine-4,4-diyl)bis(ethan-1-ol) L57-7 (440 mg, 1.49 mmol) in DCM (5 mL) was added to the reaction mixture. The reaction mixture was stirred at room temperature overnight. Formation of product was confirmed by LCMS. The reaction mixture was diluted with DCM, then washed with water and brine. The DCM layer was dried over Na₂SO₄ and concentrated under reduced pressure to give crude product. Crude product was purified by flash chromatography (SiO₂: 0-5% MeOH in DCM and 1% NH₄OH gradient) to yield Compound CY71 as colorless oil (404 mg, 26% after two steps). ¹H-NMR (300 MHz, CDCl₃) δ: 7.45 (s, 1H), 7.04 (s, 1H), 6.89 (s, 1H), 4.47 (t, J=5.3 Hz, 2H), 4.1 (t, J=7.4 Hz, 4H), 3.93 (t, J=6.75 Hz, 2H), 3.53 (m, 4H), 3.4 (m, 4H), 2.35 (m, 10H), 1.89 (m, 4H), 1.75 (m, 2H), 1.66 (m, 2H), 1.54 (m, 12H), 1.25 (m, 48H), 0.87 (m, 12H); CIMS m/z [M+H]⁺: 1004.1. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.5 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.7 min, purity: >99%.

### Synthetic Scheme for CY70

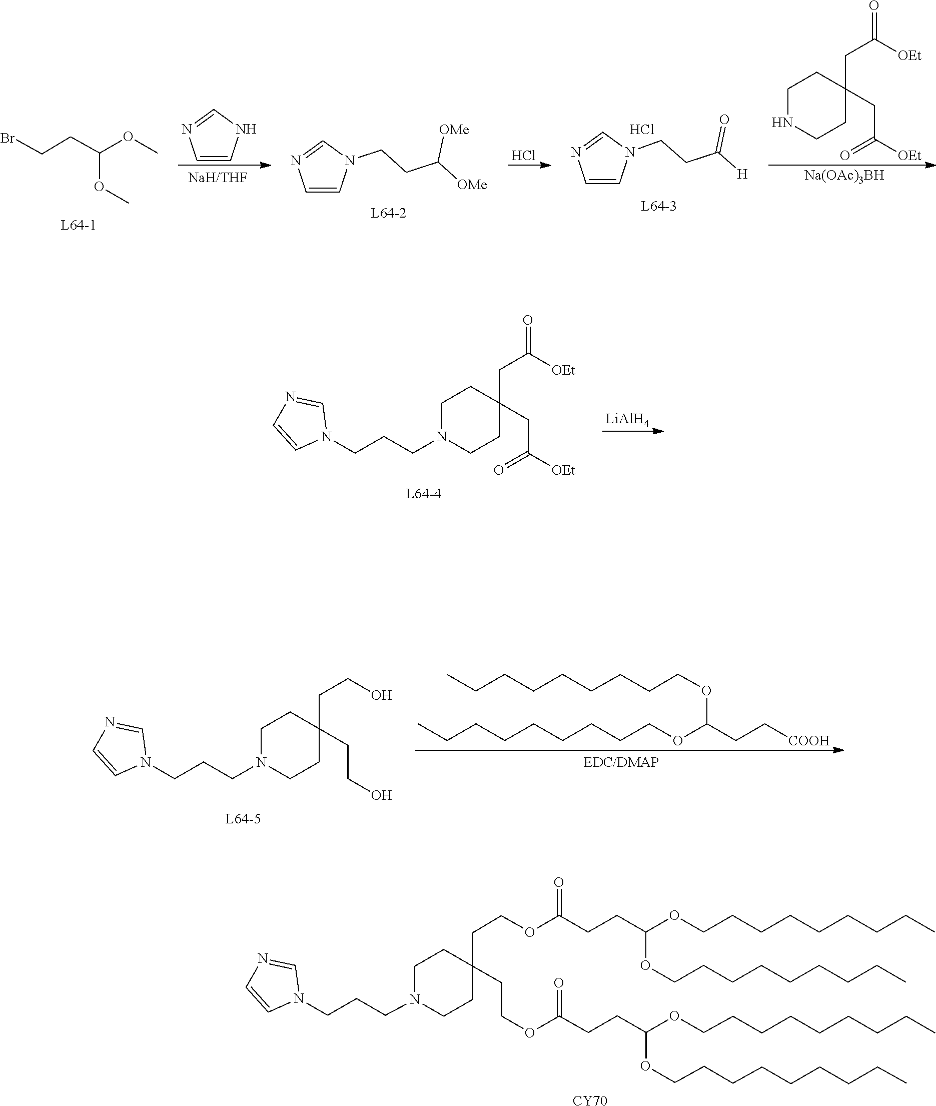

### Synthesis of 1-(3,3-dimethoxypropyl)-1H-imidazole (L64-2)

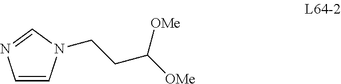 [SMILES: COC(CCn1ccnc1)OC]

To a solution of imidazole (2.0 g, 30 mmol) in anhydrous THF (60 mL) was added NaH (1.31 g, 32.78 mmol, 60% in mineral oil) portionwise with stirring. The resulting mixture was stirred at room temperature for 2h. To the suspension formed was added 3-bromo-1,1-dimethoxypropane (5.0 g, 27.32 mmol) in THF (15 mL) dropwise over a period of 10 min and further stirred for 3 h to achieve uniform mixture. The reaction mixture was heated at 60° C. overnight and then cooled to room temperature and filtered. THF was removed under reduced pressure. DCM was added followed by addition of activated charcoal and anhydrous Na₂SO₄, stirred for 2 h and filtered over celite. DCM was removed under reduced pressure to get crude product as light yellowish liquid, which was purified by flash chromatography (SiO₂: 0-5% in MeOH in DCM gradient) to yield 1-(3,3-dimethoxypropyl)-1H-imidazole L64-2 (3.64 g, 78%). ¹H-NMR (300 MHz, CDCl₃) δ: 7.45 (s, 1H), 7.04 (s, 1H), 6.9 (s, IH), 4.24 (t, J=5.49 Hz, 2H), 4.01 (t, J=7.14 Hz, 4H), 2.05 (q, J=6.84 Hz, 4H); CIMS m/z [M+H]⁺ 171.1.

### Synthesis of 3-(1H-imidazol-1-yl)propanal hydrochloride (L64-3)

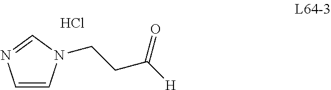

To a solution of compound 1-(3,3-dimethoxypropyl)-1H-imidazole L64-2 (3.0 g 17.64 mmol) in THF (15.0 mL), was added 1.5N HCl (15.0 mL). The reaction mixture was stirred at room temperature overnight. THF was evaporated and water layer was washed with DCM and EtOAc to remove the impurities. The aqueous layer was evaporated under reduced pressure followed by co-evaporation with acetonitrile (2×10 mL) and toluene (2×10 mL) and dried under high vacuum for 24 h to yield 3-(1H-imidazol-1-yl)propanal hydrochloride L64-3 as light-yellow gummy solid (2.7 g) which was used for the next step without further purification. ¹H-NMR (300 MHz, DMSO-D6) 6: 9.67 (s, 1H), 9.14 (s, 1H), 7.75 (s, 1H), 7.66 (s, 1H), 4.42 (t, J=6.45 Hz, 2H), 3.18 (t, J=6.6 Hz, 2H); CIMS m/z [M+H]⁺ 125.2.

### Synthesis of diethyl 2,2′-(1-(3-(1H-imidazol-1-yl)propyl)piperidine-4,4-diyl)diacetate (L64-4)

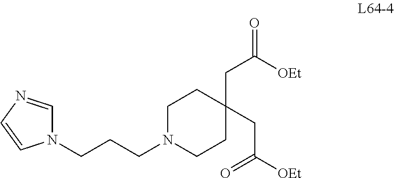 [SMILES: CCOC(=O)CC1(CC(=O)OCC)CCN(CCCn2ccnc2)CC1]

To a solution of diethyl 2,2′-(piperidine-4,4-diyl)diacetate (555 mg, 2.28 mmol) in DCE (10 mL) was added 3-(1H-imidazol-1-yl)propanal hydrochloride L64-3 (730 mg, 4.56 mmol), followed by addition of Na(OAc)₃BH (1.45 g, 6.84 mmol) and AcOH (156 mL, 2.73 mmol). The reaction mixture was stirred at room temperature under nitrogen for 18 hours and then was heated at 50° C. and stirred for 2h. Completion of the reaction was confirmed by LCMS. The reaction mixture was diluted with DCM and washed with Sat. NaHCO₃. the aqueous layer was extracted with DCM. Combined organic layers were dried over Na₂SO₄ and concentrated under reduced pressure to get crude product. Crude product was purified by flash chromatography (SiO₂: 0-6% MeOH in DCM gradient) to yield diethyl 2,2′-(1-(3-(1H-imidazol-1-yl)propyl)piperidine-4,4-diyl)diacetate L64-4 (440 mg, 52%). 1H-NMR (300 MHz, CDCl₃) δ: 7.48 (s, 1H), 7.04 (s, 1H), 6.89 (s, 1H), 4.09 (q, J=7.1 Hz, 2H), 3.99 (t, J=6.84 Hz, 4H), 2.54 (s, 4H) 2.4 (m, 4H), 2.28 (t, J=7.14 Hz, 2H), 1.92 (m, 2H), 1.69 (m, 4H), 1.26 (t, J=7.14 Hz, 6H); CIMS m/z [M+H]⁺ 366.2.

### Synthesis of 2,2′-(1-(3-(1H-imidazol-1-yl)propyl)piperidine-4,4-diyl)bis(ethan-1-ol) (L64-5)

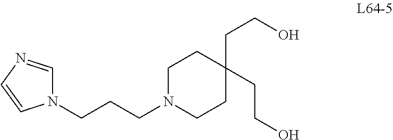 [SMILES: OCCC1(CCO)CCN(CCCn2ccnc2)CC1]

To a solution of diethyl 2,2′-(1-(3-(1H-imidazol-1-yl)propyl)piperidine-4,4-diyl)diacetate L64-4 (430 mg, 1.17 mmol) in anhydrous THF (10 mL) was added dropwise a solution of 2.OM LiAlH₄ in THF (1.2 mL, 2.35 mmol) at 0° C. The resulting mixture was stirred at room temperature overnight. The reaction mixture was cooled to 0° C. and Na₂SO₄·10H₂O was added slowly until all gas evolution stopped. After filtration through celite, the celite cake was washed with THF. All filtrates were concentrated under reduced pressure to give 2,2′-(1-(3-(1H-imidazol-1-yl)propyl)piperidine-4,4-diyl)bis(ethan-1-ol) L64-5 as colorless viscous liquid (375 mg, crude), which was used for the next step without further purification. ¹H-NMR (300 MHz, CDCl₃) δ 7.64 (s, 1H), 7.12 (s, IH), 6.91 (s, 1H), 4.05 (t, J=6.6 Hz, 2H), 3.61 (t, J=7.6 Hz, 4H), 2.45 (m, 4H), 2.32 (t, J=7.14 Hz, 2H), 1.9 (m, 2H), 1.56 (m, 8H); CIMS m/z [M+H]⁺ 282.2.

### Synthesis of (1-(3-(1H-imidazol-1-yl)propyl)piperidine-4,4-diyl)bis(ethane-2,1-diyl) bis(4,4-bis(nonyloxy)butanoate) (CY70)

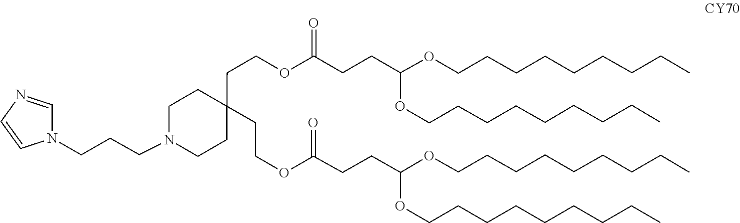 [SMILES: CCCCCCCCCOC(CCC(=O)OCCC1(CCOC(=O)CCC(OCCCCCCCCC)OCCCCCCCCC)CCN(CCCn2ccnc2)CC1)OCCCCCCCCC]

To a solution of 4,4-bis(nonyloxy)butanoic acid (L4-3(T9)) (375 mg, 1.31 mmol) in DCM (15 mL) was added DMAP (320 mg, 2.62 mmol) and EDC (1.2 g, 6.29 mmol). The reaction mixture was stirred at room temperature for 15 min, 2,2′-(1-(3-(1H-imidazol-1-yl)propyl)piperidine-4,4-diyl)bis(ethan-1-ol) L64-5 in DCM (5 mL) was added in. The reaction mixture was stirred at room temperature overnight and then diluted with DCM, washed with water and brine. DCM layer was dried over Na₂SO₄ and concentrated under reduced pressure. Crude product was purified by flash chromatography (SiO₂: 0-5% MeOH in DCM and 1% NH₄OH gradient) to yield Compound CY70 as colorless oil (445 mg, 25% after two steps). ¹H-NMR (300 MHz, CDCl₃) δ 7.45 (s, 1H), 7.04 (s, IH), 6.89 (s, 1H), 4.48 (t, J=5.4 Hz, 2H), 4.11 (t, J=7.4 Hz, 4H), 3.99 (t, J=7.17 Hz, 2H), 3.54 (m, 4H), 3.4 (m, 4H), 2.36 (m, 8H), 2.25 (m, 2H), 1.92 (m, 6H), 1.67 (m, 2H), 1.52 (m, 10H), 1.25 (m, 50H), 0.87 (m, 12H); CIMS m/z [M+H]⁺ 990.1. Analytical HPLC column: Agilent Zorbax SB-C18, 5 μm, 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.3 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.4 min, purity: >99%.

### Synthesis of Compound C5

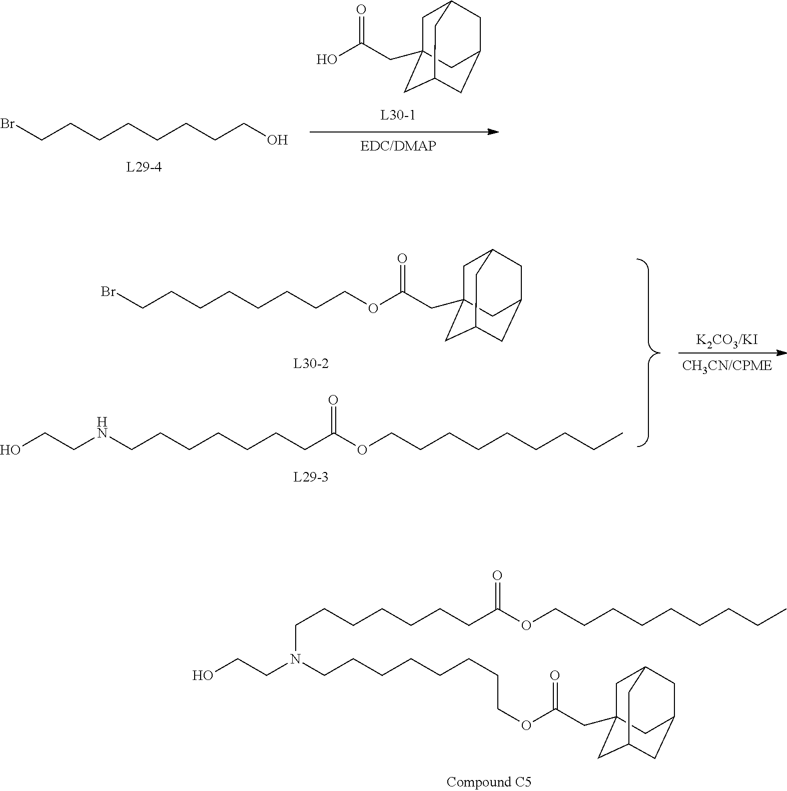 [SMILES: O=C(O)CC12CC3CC(CC(C3)C1)C2 ; OCCCCCCCCBr ; O=C(CC12CC3CC(CC(C3)C1)C2)OCCCCCCCCBr ; CCCCCCCCCOC(=O)CCCCCCCNCCO ; CCCCCCCCCOC(=O)CCCCCCCN(CCO)CCCCCCCCOC(=O)CC12CC3CC(CC(C3)C1)C2]

### Synthesis of 8-bromooctyl 2-((3r,5r,7r)-adamantan-1-yl)acetate (L30-2)

To a solution of compound L30-1 (3.0 g, 15.4 mmol) in dichloromethane (100 mL) was added DMAP (1.89 g, 15.4 mmol), EDC (17.7 g, 92.6 mmol) and compound L29-4 (8.0 g, 38.6 mmol). The reaction mixture was stirred at room temperature for 12 h and reduced under vacuum. The residue was dissolved in dichloromethane (300 mL) and washed with brine (80 mL×3). The organic phase was dried over anhydrous Na₂SO₄, the solvent was reduced under vacuum, and the crude was purified by column chromatography (330 g SiO₂: 0 to 30% Ethyl acetate in Hexane gradient) to obtain compound L30-2 as colorless oil (4.5 g, 83%). ¹H-NMR (300 MHz, CDCl₃) δ 4.03 (t, 2H), 3.40 (t, 2H), 2.05 (s, 2H), 1.96-1.92 (m, 2H), 1.88-1.81 (m, 2H), 1.71-1.56 (m, 15H), 1.43-1.35 (m, 8H); CIMS m/z [M+H]⁺ 385.3.

### Synthesis of nonyl 8-((8-(2-((3r,5r,7r)-adamantan-1-yl)acetoxy)octyl)(2-hydroxyethyl) amino)octanoate (Compound C5)

To a solution of compound L29-3 (0.35 g, 1.06 mmol) in CPME (10 mL) and CH₃CN 10 mL), under nitrogen, was added compound L30-2 (0.37 g, 1.16 mmol), K₂CO₃ (0.587 g, 4.2 mmol) and KI (0.176 g, 1.09 mmol). The reaction mixture was heated at 60° C. for 18 h. After cooling to room temperature, the reaction mixture was filtered through celite, washed with ethyl acetate, and the solvent removed under vacuum to give the crude product which was purified by flash chromatography (SiO₂: 5% triethylamine in hexane/ethyl acetate 0-25%) to obtain Compound 5 as colorless oil (0.33 g, 52%). ¹H-NMR (300 MHz, CDCl₃) δ 4.06-4.0 (m, 4H), 3.51 (t, 2H), 2.55 (t, 2H), 2.42 (t, 3H), 2.28 (t, 2H), 2.05 (s, 2H), 1.98-1.94 (m, 3H), 1.71-1.57 (m, 18H), 1.43-1.25 (m, 32H), 0.87 (t, 3H)); CIMS m/z [M+H]⁺ 634.1. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=4.33 min, purity: >97%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=10.16 min, purity: 90.2%.

### Synthesis of Compound C8

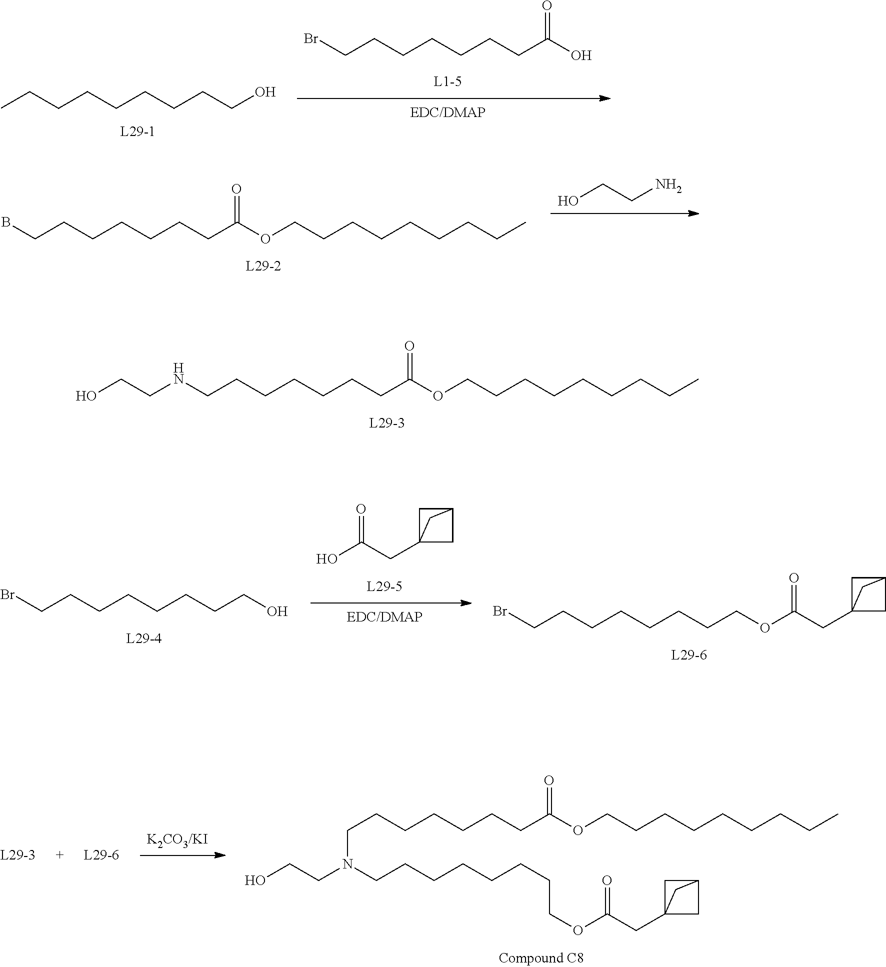

### Synthesis of nonyl 8-bromooctanoate (L29-2)

To a solution of compound L1-5 (3.0 g, 12.8 mmol) in dichloromethane (100 mL) was added DMAP (1.5 g, 12.8 mmol), EDC (9.86 g, 51.4 mmol) and L29-1 (4.08 g, 28.3 mmol). The reaction mixture was stirred at room temperature for 12 h and reduced under vacuum. The residue was dissolved in dichloromethane (300 mL) and washed with brine (80 mL×3). The organic phase was dried over anhydrous Na₂SO₄, the solvent was reduced under vacuum, and the crude was purified by column chromatography (SiO₂: 0 to 30% Ethyl acetate in Hexane gradient) to give compound L29-2 as colorless oil (2.5 g, 55%). ¹H-NMR (300 MHz, CDCl₃) δ 4.09 (t, 2H), 3.59 (t, 2H), 2.69 (t, 2H), 1.82-1.59 (m, 6H), 1.45-1.27 (m, 18H), 0.85 (t, 3H); CIMS m/z [M+H]⁺ 349.3.

### Synthesis of nonyl 8-((2-hydroxyethyl)amino)octanoate (L29-3)

To a solution of compound L29-2 (2.5 g, 7.1 mmol) in ethanol (40 mL) was added dropwise a solution of ethanolamine (6.55 g, 107 mmol) in EtOH (20 mL) at ambient temperature. The reaction solution was heated at 60° C.-70° C. for 12 h and concentrated in vacuo to give crude residue which was diluted with methyl tert-butyl ether (TBME). The TBME layer was separated from ethanolamine layer and the ethanolamine layer was back extracted with TBME (200 mL). The combined TBME layers were washed with 5% NaHCO₃ solution and then concentrated in vacuo at 40° C. to give the crude product which was purified by flash chromatography (SiO₂: DCM/MeOH 0-100%, 1% NH₄OH) to afford L29-3 as colorless oil (1.3 g, 55%). ¹H-NMR (300 MHz, CDCl₃) δ 4.02 (t, 2H), 3.61 (t, 2H), 2.72 (t, 2H), 2.57 (t, 2H), 2.46 (s, 2H), 2.25 (t, 2H), 1.66-1.40 (m, 6H), 1.35-1.13 (m, 18H), 0.84 (t, 3H); CIMS m/z [M+H]⁺ 330.3.

### Synthesis of 8-bromooctyl 2-(bicyclo[1.1.1]pentan-1-yl)acetate (L29-6)

To a solution of compound L29-5 (1.0 g, 7.9 mmol) in dichloromethane (30 mL) was added DMAP (0.97 g, 7.9 mmol, 1 eq) and EDC (6.0 g, 31.7 mmol) and compound L29-4 (3.6 g, 17.4 mmol). The reaction mixture was stirred at room temperature for 12 h and then concentrated under vacuum. The residue was dissolved in dichloromethane (300 mL) and washed with brine (80 mL×3). The organic phase was dried over anhydrous Na₂SO₄, the solvent was reduced under vacuum, and the crude was purified by column chromatography (SiO₂: 0 to 30% Ethyl acetate in Hexane gradient) to give compound L29-6 as colorless oil (0.75 g, 30%). ¹H-NMR (300 MHz, CDCl₃) δ 4.04 (t, 2H), 3.39 (t, 2H), 2.48-2.40 (m, 3H), 1.91-1.72 (m, 8H), 1.68-1.53 (m, 2H), 1-48-1.24 (m, 8H); CIMS m/z 317.5 [M+H]⁺ 317.5.

### Synthesis of nonyl 8-((8-(2-(bicyclo[1.1.1]pentan-1-yl)acetoxy)octyl)(2-hydroxyethyl)amino)octanoate (C8)

To a solution of compound L29-3 (0.35 g, 1.06 mmol) in CPME (10 mL) and ACN (10 mL), under nitrogen, was added compound L29-6 (0.37 g, 1.16 mmol), K₂CO₃ (0.587 g, 4.2 mmol) and KI (0.176 g, 1.09 mmol). The reaction mixture was heated at 80° C. for 18 h. After cooling to room temperature, the reaction mixture was filtered through celite, and washed with ethyl acetate. The combined filtrates were concentrated under vacuum to give the crude product which was purified by flash chromatography (SiO₂: 5% triethylamine in hexane/ethyl acetate 0-25%) to obtain compound C₈ as colorless oil (0.31 g, 51%). ¹H-NMR (300 MHz, CDCl₃) δ 4.04 (t, 4H), 3.50 (t, 2H), 2.55 (t, 2H), 2.48-2.40 (m, 7H), 2.27 (t, 2H), 1.77 (s, 6H), 1.65-1.18 (m, 37H), 0.86 (t, 3H)); CIMS m/z [M+H]⁺ 711.3. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=7.62 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=9.37 min, purity: 94.4%.

### Synthesis of Compound C13

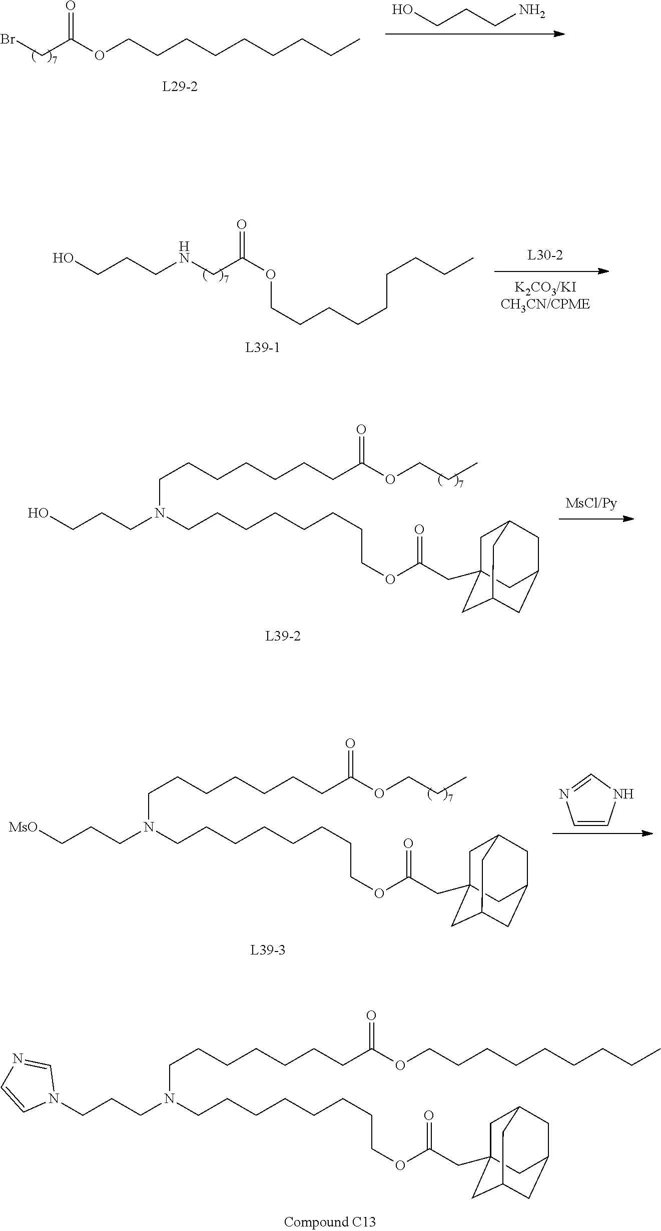

### Synthesis of nonyl 8-((3-hydroxypropyl)amino)octanoate (L39-1)

To a solution of compound L29-2 (1.5 g, 4.2 mmol) in ethanol (20 mL) was added a solution of propanolamine (4.8 g, 64.0 mmol) in EtOH (10 mL) at ambient temperature. The reaction solution was heated at 70° C. for 12 h and concentrated in vacuo at 40° C. to give crude residue. The crude residue was diluted with TBME and then the TBME layer was separated from the ethanolamine layer. The ethanolamine layer was extracted again with TBME (200 mL). The combined TBME layers were washed with 5% NaHCO₃ solution and then concentrated in vacuo at 40° C. to give pale yellow oil. The crude product was purified by flash chromatography (SiO₂: DCM/MeOH 0-100%, 1% NH₄OH) to obtain compound L39-1 as colorless oil (1.1 g, 74%). ¹H-NMR (300 MHz, CDCl₃) δ 4.03 (t, 2H), 3.63 (t, 2H), 2.70 (t, 2H), 2.58 (t, 2H), 2.46 (t, 2H), 2.24 (t, 2H), 1.62-1.41 (m, 6H), 1.36-1.14 (m, 20H), 0.85 (t, 3H); CIMS m/z 343.5 [M+H]⁺.

### Synthesis of nonyl 8-((8-(2-((3r,5r,7r)-adamantan-1-yl)acetoxy)octyl)(3-hydroxypropyl) amino)octanoate (L39-2)

To a solution of compound L39-1 (1.0 g, 2.9 mmol) in CH₃CN/CPME (1:1, 30 mL) under nitrogen, was added L30-2 (1.3 g, 3.5 mmol) and followed by the addition of K₂CO₃ (1.6 g, 11.6 mmol) and KI (0.483 g, 2.9 mmol). The reaction mixture was heated at 100° C. for 18 h. After cooling to room temperature, the reaction mixture was filtered through celite, washed with ethyl acetate, and the solvent removed under vacuum to give the crude product which was purified by flash chromatography (SiO2: 5% triethylamine in hexane/ethyl acetate 0-25%) to give L39-2 as colorless oil (1.2 g, 64%). ¹H-NMR (300 MHz, CDCl₃) δ 5.75 (s, 1H), 4.05-3.95 (m, 6H), 3.77 (t, 2H), 3.36 (t, 2H), 2.61 (t, 2H), 2.37 (t, 2H), 2.27 (t, 2H), 2.04 (s, 2H), 1.95 (s, 2H), 1.74-1.09 (m, 48H), 0.86 (t, 3H); CIMS m/z 698.1 [M+H]⁺.

### Synthesis of nonyl 8-((8-(2-((3r,5r,7r)-adamantan-l-yl)acetoxy)octyl)(3-hydroxypropyl) amino)octanoate (L39-3)

To a solution of compound L39-2 (1.2 g, 1.85 mmol) in DCM (30 mL) under nitrogen was added MsCl (0.254 g, 2.2 mmol) and triethylamine (0.468 g, 4.62 mmol). The reaction mixture was stirred at room temperature for 2 h and reduced under vacuum. The residue was dissolved in dichloromethane (300 mL) and washed with brine (80 mL×3). The organic phase was dried over anhydrous Na₂SO₄, the solvent was reduced under vacuum, and the crude L39-3 was obtained as colorless oil (1.0 g, 74%) which was used for next step without further purification.

### Synthesis of (nonyl 8-((3-(IH-imidazol-1-yl)propyl)(8-(2-((3r,5r,7r)-adamantan-1-yl)acetoxy)octyl)amino)octanoate) (Compound C13)

To a solution of compound L39-3 (1.0 g, 1.37 mmol) in 2-propanol (30 mL) under nitrogen, imidazole (1.87 g, 27.5 mmol) was added. The reaction mixture was stirred at room temperature for 2 h and then heated at 90° C. for 12 h. After cooling to room temperature, the reaction mixture was dissolved in dichloromethane (300 mL) and washed with brine (80 mL×3). The organic phase was dried over anhydrous Na₂SO₄, the solvent was removed under vacuum to give the crude product which was purified by flash chromatography (SiO₂: DCM/MeOH 0-5%) to get Compound C13 as colorless oil (0.2 g, 20%). ¹H-NMR (300 MHz, CDCl₃) δ 7.46 (s, 1H), 7.04 (s, 11H), 6.91 (s, IH), 4.06-3.97 (m, 6H), 2.59-2.35 (m, 6H), 2.27 (t, 2H), 2.04 (s, 2H), 1.98-1.89 (m, 5H), 1.74-1.59 (m, 18H), 1.40-1.12 (m, 30H), 0.86 (t, 3H); CIMS m/z [M+H]⁺ 698.51. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.33 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=8.01 min, purity: 86.4%.

### Synthesis of Compound C21

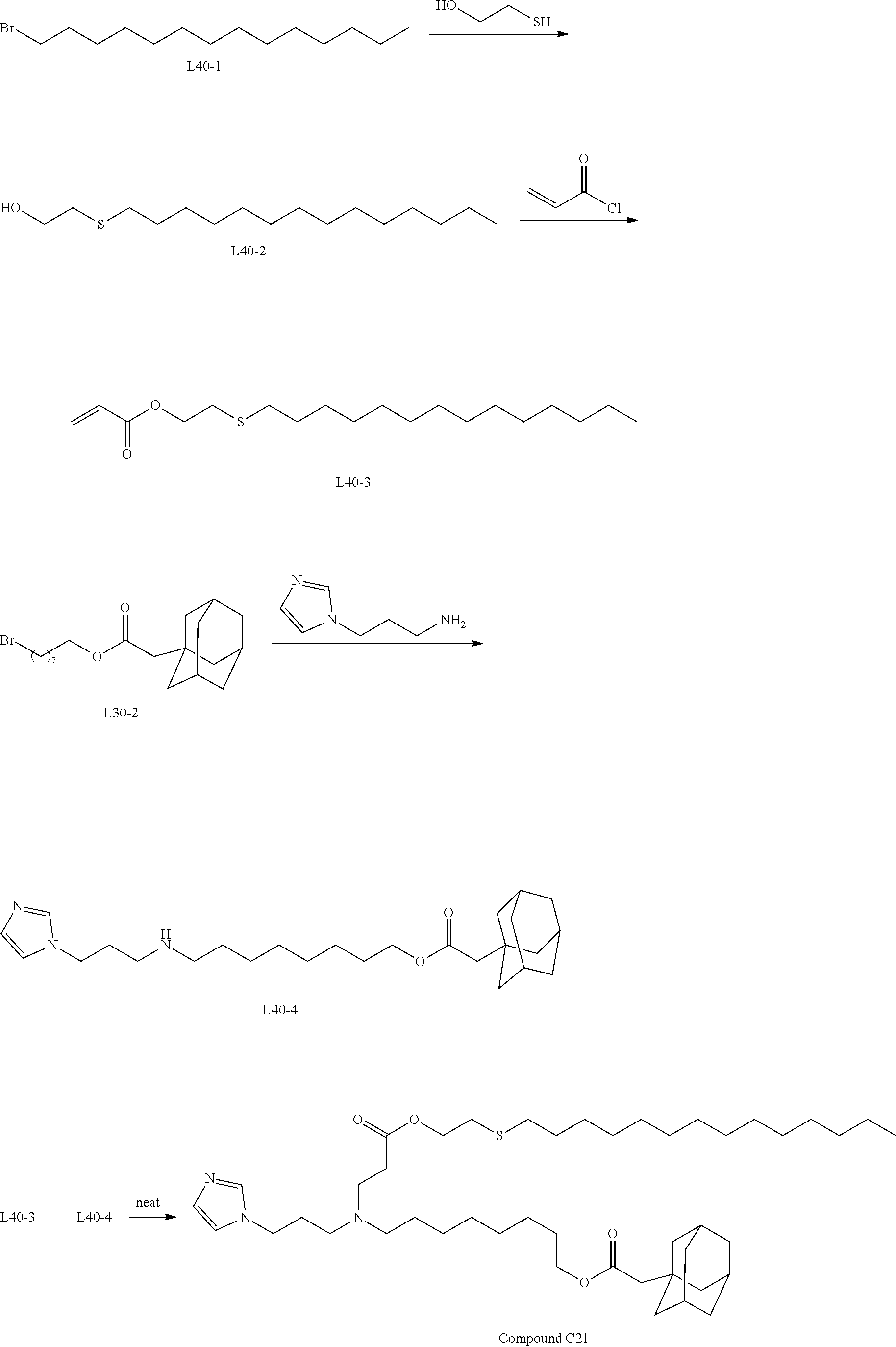

### Synthesis of 2-(tetradecylthio)ethan-1-ol (L40-2)

To a solution of L40-1 (7 g, 1 eq) in DMF (50 mL) was added K₂CO₃ (7 g, 2 eq) followed by the addition of 2-mercaptoethan-1-ol (2 mL, 1.2 eq). The resulting mixture was stirred for 16 hours at room temperature. The reaction mixture was then filtered through filter paper and the filter cake was rinsed with DCM (30 mL). The organic fraction was reduced under vacuum to obtain crude product which was subjected to silica gel column using 0-20% EA in hexane as eluent to afford L40-2 as white solid (5.7 g, 85%). ¹H-NMR (300 MHz, CDCl₃) δ 3.70 (q, 2H), 2.72 (t, 2H), 2.50 (t, 2H), 2.14 (m, 1H), 1.54-1.59 (m, 2H), 1.34-1.25 (m, 22H), 0.87 (t, 3H).

### Synthesis of 2-(tetradecylthio)ethyl acrylate (L40-3)

The starting material L40-2 (2 g, 1 eq) and triethylamine (1.6 mL, 1.5 eq) were dissolved in DCM (40 mL). The solution was cooled to 0° C. and acryloyl chloride (800 μL, 1.2 eq) was added in dropwise. The reaction mixture was then allowed to warm to room temperature and stirred overnight. The reaction mixture was washed with H₂O (100 mL) and brine (100 mL). The organic layer was dried over Na₂SO₄ and reduced under vacuum to obtain crude product which was subjected to silica gel column using 0-10% EA in hexane as eluent to afford L40-3 as colorless oil (1.6 g, 70%). ¹H-NMR (300 MHz, CDCl₃) δ 6.40 (dd, 1H), 6.07-6.16 (qd, 1H), 5.82 (dd, 1H), 4.29 (t, 2H), 2.76 (t, 2H), 2.55 (t, 2H), 1.54-1.59 (m, 2H), 1.34-1.25 (m, 22H), 0.87 (t, 3H).

### Synthesis of 8-((3-(1H-imidazol-1-yl)propyl)amino)octyl 2-((3r,5r,7r)-adamantan-1-yl)acetate (L40-4)

1-(3-Aminopropyl)imidazole (1.74 g, 10 eq) was dissolved in ethanol, followed by adding the starting material L30-2 (500 mg, 1 eq). The reaction mixture was stirred at 80° C. for 16 hours. When TLC showed completion of the reaction, the solvent was reduced under vacuum and the crude product was dissolved in a small amount of DCM and subjected to silica gel column using 0-40% MeOH in DCM with 2% NH₃·H₂O as eluent to afford L40-4 as colorless oil (360 mg, 60%). ¹H-NMR (300 MHz, CDCl₃) δ 7.58 (s, 1H), 7.14 (s, 1H), 6.86 (s, 1H), 4.00-3.96 (m, 4H), 2.50-2.37 (m, 4H), 2.02 (s, 2H), 1.91 (bs, 3H), 1.82-1.77 (m, 2H), 1.76-1.48 (m, 12H), 1.40-1.20 (m, 9H); CIMS m/z [M+H]⁺ 430.3.

### Synthesis of 2-(tetradecylthio)ethyl 3-((3-(1H-imidazol-1-yl)propyl)(8-(2-((3r,5r,7r)-adamantan-1-yl)acetoxy)octyl)amino)propanoate (Compound C21)

The starting materials L40-4 (360 mg, 1.2 eq) and L40-3 (229 mg, 1 eq) obtained from previous steps were mixed in a round bottom flask. The reaction was stirred at 90° C. for 24 hours. The reaction mixture was then subjected to silica gel column using 0-6% MeOH in DCM as eluent to afford Compound C21 (254 mg, 48%) as light-yellow oil. ¹H-NMR (300 MHz, DMSO-d₆) δ, 7.58 (s, 1H), 7.14 (s, 1H), 6.87 (s, 1H), 4.13 (t, 2H), 4.04-3.84 (m, 4H), 2.69 (t, 2H), 2.63 (t, 2H), 2.37 (t, 2H), 2.32-2.22 (m, 4H), 2.02 (s, 2H), 1.91 (bs, 3H), 1.84-1.72 (m, 2H), 1.71-1.42 (m, 18H), 1.38-1.12 (m, 36H), 0.85 (t, 3H); CIMS m/z [M+H]⁺ 758.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 15 min, flow rate: 1 mL/min, column temperature: 20±2° C., detector: ELSD, t_{R}=5.94 min, purity: 99.2%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 20 min, flow rate: 1 mL/min, column temperature: 20±2° C., detector: CAD, t_{R}=6.98 min, purity: 98.0%.

### Synthesis of Compound C51

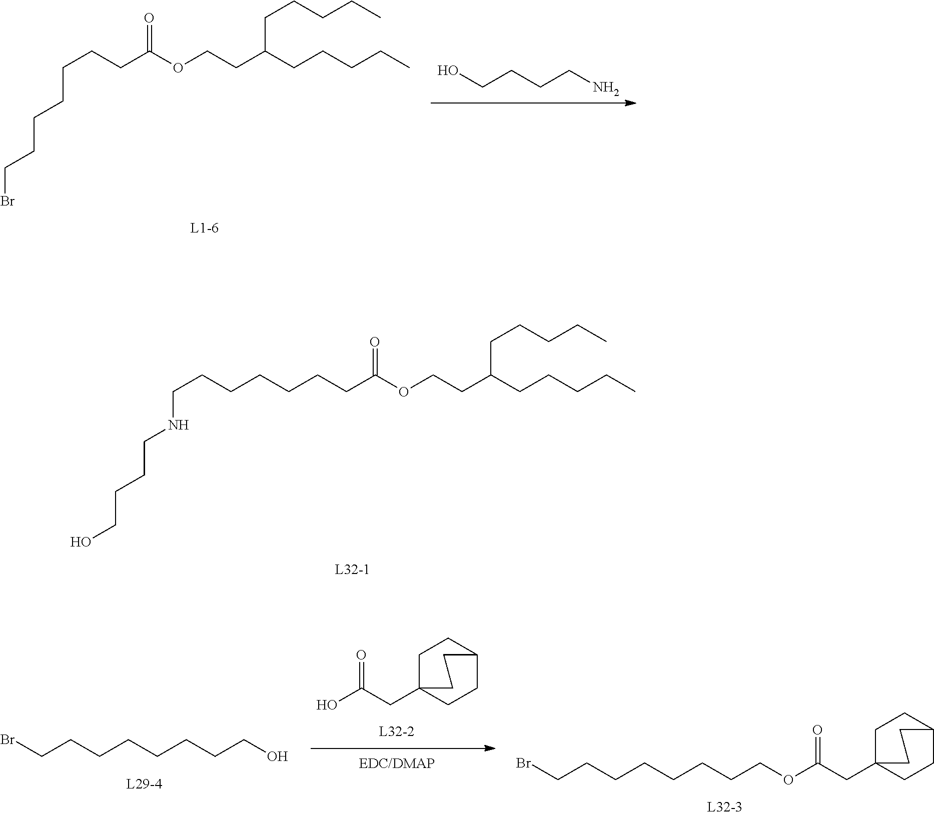

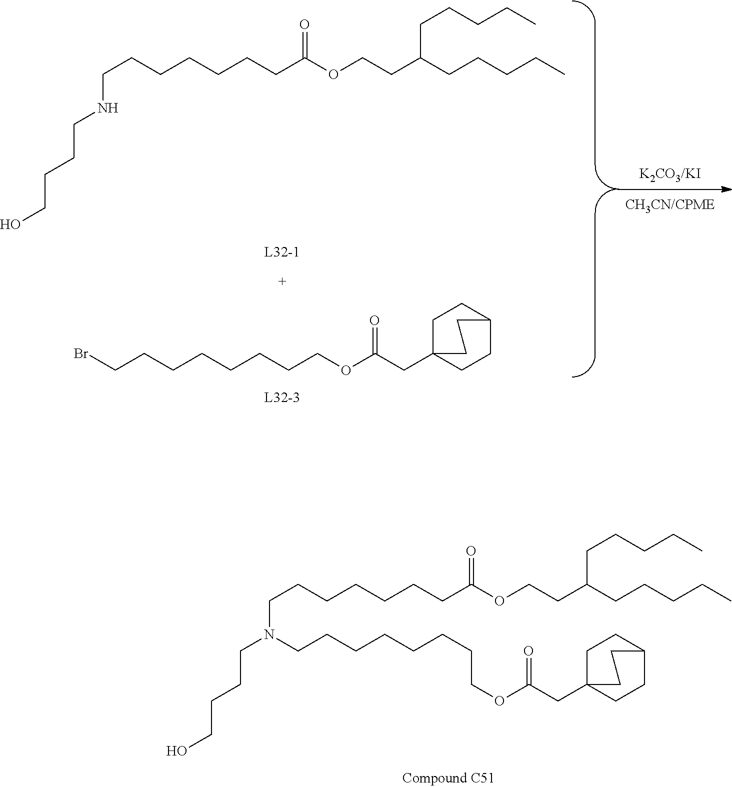 [SMILES: CCCCCC(CCCCC)CCOC(=O)CCCCCCCNCCCCO ; O=C(CC12CCC(CC1)CC2)OCCCCCCCCBr ; CCCCCC(CCCCC)CCOC(=O)CCCCCCCN(CCCCO)CCCCCCCCOC(=O)CC12CCC(CC1)CC2]

### Synthesis of 3-pentyloctyl 8-((4-hydroxybutyl)amino)octanoate (L32-1)

To a solution of 3-pentyloctyl 8-bromooctanoate L1-6 (6.0 g, 14.8 mmol) in ethanol (120 mL) was added 4-aminobutanol (13.1 g, 148.1 mmol). The resulting mixture was heated to 65-70° C. and stirred overnight. The reaction mixture was then concentrated. DCM (250 mL) was added to the residue. The solution was washed with water (100 mL), brine and dried over Na₂SO₄. Concentration under reduced pressure gave crude product, which was purified flash chromatography (SiO₂: 0 to 10% MeOH in DCM (1% NH₄OH) gradient) to yield L32-1 as colorless oil (4.44 g, 72%). ¹H-NMR (300 MHz, CDCl₃) δ 4.06 (t, 2H), 3.56 (t, 2H), 2.57-2.64 (m, 4H), 2.26 (t, 2H), 1.49-1.67 (m, 11H), 1.24-1.30 (m, 24H), 0.87 (t, 6H); CIMS m/z [M+H]⁺ 414.4.

### Synthesis of 8-bromooctyl 2-(bicyclo[2.2.2]octan-1-yl)acetate (L32-3)

To a solution of compound L32-2 (1.0 g, 5.9 mmol) in dichloromethane (20 mL) was added DMAP (0.729 g, 5.9 mmol), EDC (4.55 g, 23.7 mmol) and L29-4 (2.73 g, 13.0 mmol). The reaction mixture was stirred at room temperature for 12 h and reduced under vacuum. The residue was dissolved in dichloromethane (300 mL) and washed with brine (80 mL×3). The organic phase was dried over anhydrous Na₂SO₄, the solvent was removed under vacuum and the crude product was purified by flash chromatography (SiO₂: 0 to 30% Ethyl acetate in Hexane gradient) to get product L32-3 as colorless oil (0.72 g, 33%). ¹H-NMR (300 MHz, CDCl₃) δ 4.08 (t, 2H), 3.55 (t, 2H), 2.09 (s, 2H), 1.80 (m, 2H), 1.61-1.24 (m, 23H); CIMS m/z [M+H]⁺ 359.3.

### Synthesis of 3-pentyloctyl 8-((8-(2-(bicyclo[2.2.2]octan-1-yl)acetoxy)octyl)(4-hydroxybutyl)amino)octanoate (Compound C51)

To a solution of compound L32-1 (0.33 g, 0.79 mmol) in CH₃CN/CPME (1:1, 10 mL) under nitrogen, was added compound L32-3 (0.315 g, 0.87 mmol), followed by the addition of K₂CO₃ (0.44 g, 3.1 mmol) and KI (0.13 g, 0.79 mmol). The reaction mixture was heated at 60° C. for 18 h. After cooling to room temperature, the reaction mixture was filtered through celite, washed with ethyl acetate, and the solvent removed under vacuum to give the crude product, which was purified by flash chromatography (SiO₂: 5% triethylamine in hexane/ethyl acetate 0-25%) to obtain Compound C51 as colorless oil (0.25 g, 45%). ¹H-NMR (300 MHz, CDCl₃) δ 4.09-3.98 (m, 4H), 3.59-3.38 (m, 2H), 2.51-2.39 (m, 6H), 2.27 (t, 2H), 2.03 (s, 2H), 1.72-1.15 (m, 61H), 0.87 (t, 6H)); CIMS m/z [M+H]⁺ 692.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.36 min, purity: >96%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=12.95 min, purity: >85.4%.

### Synthesis of Compound C52

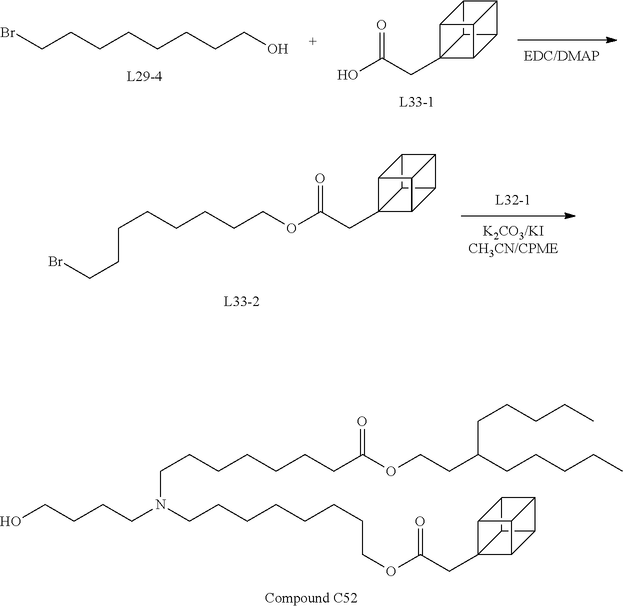 [SMILES: OCCCCCCCCBr ; O=C(O)CC12C3C4C5C3C1C5C42 ; O=C(CC12C3C4C5C3C1C5C42)OCCCCCCCCBr ; CCCCCC(CCCCC)CCOC(=O)CCCCCCCN(CCCCO)CCCCCCCCOC(=O)CC12C3C4C5C3C1C5C42]

### Synthesis of 8-bromooctyl 2-((2r,3r,5r,6r,7r,8r)-cuban-1-yl)acetate (L33-2)

A mixture of L33-1 (990 mg, 6.165 mmol), EDC (4.68 g, 24.41 mmol) and DMAP (820.3 mg, 6.71 mmol) in dichloromethane (20 mL) was stirred at room temperature for 10 min. L29-4 (1.91 g, 9.15 mmol) was added and the resulting mixture was stirred at room temperature for 4 h. The reaction mixture was concentrated under reduced pressure and the residue was purified by flash chromatography (SiO₂: ethyl acetate/hexane 0-10%) to give Compound L33-2 (1.81 g, 84%) as slightly yellow oil. ¹H NMR (300 MHz, CDCl₃): δ ppm 4.07-4.02 (m, 3H), 3.98-3.81 (m, 6H), 3.39 (t, J=7.0 Hz, 2H), 2.63 (s, 2H), 1.87-1.79 (m, 2H), 1.60-1.54 (m, 2H), 1.43-1.31 (m, 8H). MS (CI): m/z [M+H]⁺ 354.42

### Synthesis of 3-pentyloctyl 8-((8-(2-((2r,3r,5r,6r,7r,8r)-cuban-1-yl)acetoxy)octyl)(4-hydroxy butyl)amino)octanoate (C52)

A mixture of L33-2 (600 mg, 1.69 mmol), L32-1 (351.3 mg, 0.849 mmol), KI (423 mg, 2.55 mmol) and potassium carbonate (704 mg, 5.09 mmol) in anhydrous ACN (5 mL) and cyclopentylmethyl ether (CPME) (5 mL) was stirred at 90° C. for 36 h. After cooling to room temperature, the reaction mixture was filtered through celite. The celite cake was washed with ACN (15 ml). The combined filtrates were concentrated. The obtained crude was purified by flash chromatography (SiO₂: ethyl acetate/hexane 0-50%, the flash column was equilibrated with 10% triethylamine in hexane for 15 min before use) to give Compound C₅₂ (770 mg, 66%). ¹H NMR (300 MHz, CDCl₃): δ ppm 6.7 (bs, 1H), 4.07-4.0 (m, 6H), 3.98-3.81 (m, 6H), 3.61 (t, J=2.0 Hz, 2H), 2.63 (s, 2H), 2.43-2.2.39 (m, 6H), 2.31 (t, J=5.0 Hz, 2H), 1.65-1.19 (m, 44H), 0.85 (t, J=7.0 Hz, 6H). MS (CI): m/z [M+H]⁺ 687.09. Analytical HPLC column: Agela Durashell C18, 3 μm (Catalog No. DC930505-0), 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >90.72%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.1 min, purity: 97.3%.

### Synthesis of Compound C53

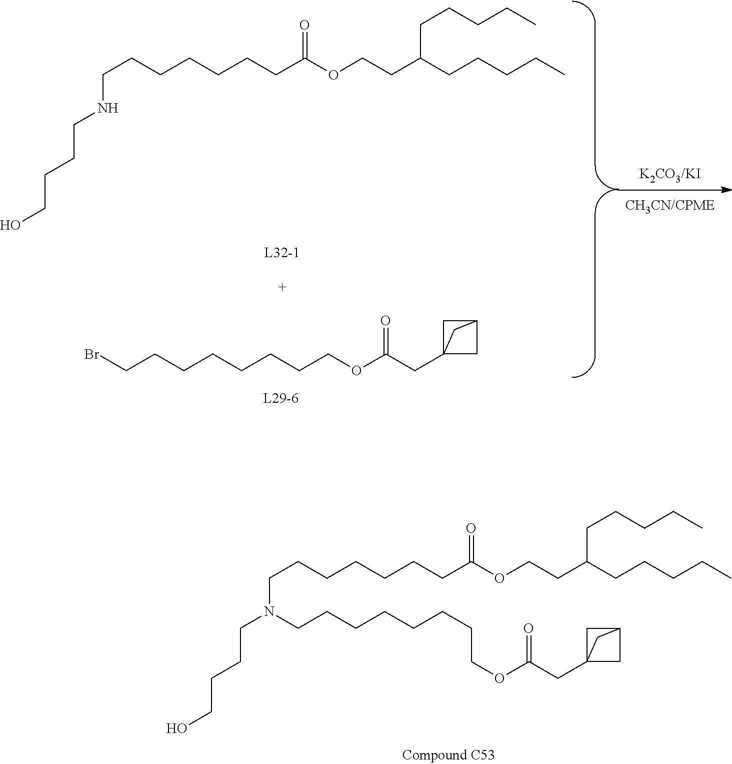 [SMILES: CCCCCC(CCCCC)CCOC(=O)CCCCCCCNCCCCO ; O=C(CC12CC(C1)C2)OCCCCCCCCBr ; CCCCCC(CCCCC)CCOC(=O)CCCCCCCN(CCCCO)CCCCCCCCOC(=O)CC12CC(C1)C2]

### Synthesis of ((3-pentyloctyl 8-((8-(2-(bicyclo[1.1.1]pentan-1-yl)acetoxy)octyl)(4-hydroxybutyl)amino)octanoate) (Compound 53)

To a solution of compound L32-1 (0.35 g, 0.84 mmol) in CH₃CN/CPME (1:1, 10 mL) under nitrogen was added L29-6 (0.322 g, 1.01 mmol), followed by the addition of K₂CO₃ (0.467 g, 3.3 mmol) and KI (0.14 g, 0.84 mmol). The reaction mixture was heated at 100° C. for 18 h. After cooling to room temperature, the reaction mixture was filtered through Celite, washed with ethyl acetate, and the solvent removed under vacuum to give the crude product which was purified by flash chromatography (SiO₂: 5% triethylamine in hexane/ethyl acetate 0-25%) to obtain Compound C53 as colorless oil (0.315 g, 57%). ¹H-NMR (300 MHz, CDCl₃) δ 4.06 (t, 4H), 3.59-348 (m, 2H), 2.46-2.38 (m, 8H), 2.26 (t, 2H), 1.77 (s, 6H), 1.63-1.08 (m, 47H), 0.86 (t, 6H)); CIMS m/z [M+H]⁺ 650.5. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=6.66 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=7.52 min, purity: >94.1%.

### Synthesis of Compound C55

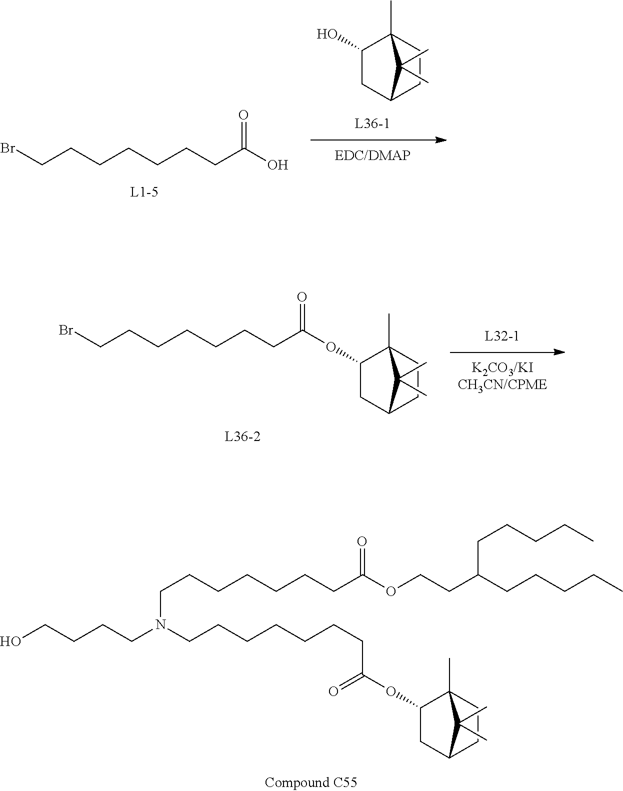 [SMILES: CC1(C)[C@@H]2CC[C@@]1(C)[C@@H](O)C2 ; O=C(O)CCCCCCCBr ; CC1(C)[C@@H]2CC[C@@]1(C)[C@@H](OC(=O)CCCCCCCBr)C2 ; CCCCCC(CCCCC)CCOC(=O)CCCCCCCN(CCCCO)CCCCCCCC(=O)O[C@H]1C[C@H]2CC[C@]1(C)C2(C)C]

### Synthesis of (1R,2S,4R)-1,7,7-trimethylbicyclo[2.2.1]heptan-2-yl 8-bromooctanoate (L36-2)

To a solution of 1R-(+)-Borneol L36-1 (1.0 g, 6.49 mmol), 8-bromo octanoic acid L1-5 (1.73 g, 7.78 mmol) and DMAP (395 mg, 3.24 mmol) in DCM (20 mL) was added EDC (2.97 g, 15.5 mmol) at room temperature and the resulting mixture was stirred under nitrogen atmosphere overnight. The reaction mixture was concentrated to half the volume and purified by flash chromatography (SiO₂: 0 to 5% ethyl acetate in hexane gradient) to yield L36-2 as colorless oil (1.7 g, 73%). ¹H-NMR (300 MHz, CDCl₃) δ 4.86 (m, 1H), 3.37-3.52 (m, 2H), 2.31 (m, 3H), 1.6-1.87 (m, 8H), 1.25-1.43 (m, 8H), 0.8-0.89 (m, 9H).

### Synthesis of 3-pentyloctyl 8-((4-hydroxybutyl)(8-oxo-8-(((1R,2S,4R)-1,7,7-trimethylbicyclo [2.2.1]heptan-2-yl)oxy)octyl)amino)octanoate (Compound C55)

To a solution of L32-1 (500 mg, 1.21 mmol) and L36-2 (520 mg, 1.45 mmol) in a mixture of AcCN/CPME (1:1, 10 mL) under nitrogen was added K₂CO₃ (500 mg, 3.63 mmol) and KI (100 mg, 0.605 mmol). The reaction mixture was heated at 75° C. overnight under nitrogen atmosphere. After cooling to room temperature, the reaction mixture was filtered through celite. The filtrate was concentrated to give crude product, which was purified by flash chromatography (SiO₂: 0 to 5% MeOH in DCM gradient) to yield Compound C55 as colorless oil (397 mg, 47%). ¹H-NMR (300 MHz, CDCl₃) δ 4.87 (m, 1H), 4.06 (t, J=7.14 Hz, 2H), 3.55 (m, 2H), 2.48 (m, 6H), 2.27 (m, 4H), 1.92 (m, 1H), 1.52-1.73 (m, 16H), 1.18-1.3 (m, 34H) 0.81-0.89 (m, 15H); CIMS m/z [M+H]⁺ 692.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=6.67 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=9.16 min, purity: >99%.

### Synthesis of Compound C56

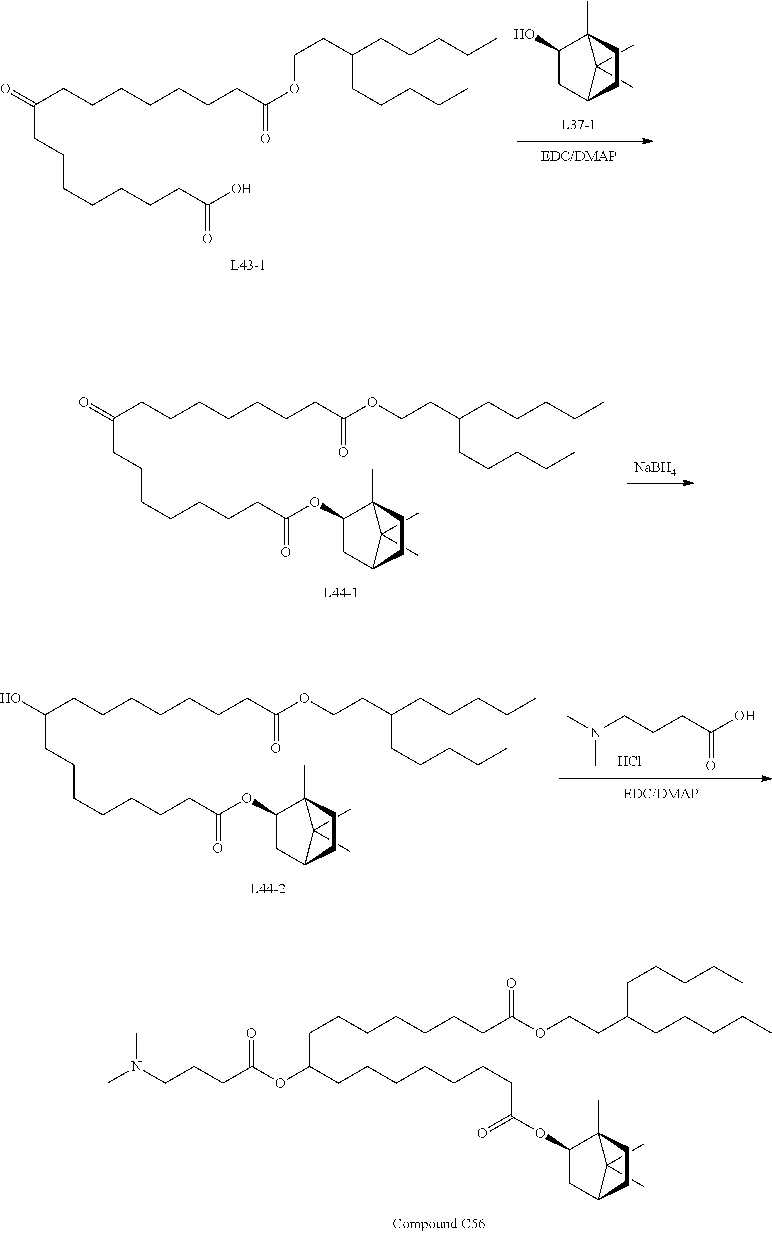

### Synthesis of 1-(3-pentyloctyl) 17-((1S,2R,4S)-1,7,7-trimethylbicyclo[2.2.1]heptan-2-yl) 9-oxoheptadecanedioate (L44-1)

A mixture of L43-1 (780 mg, 1.57 mmol), EDC (1.2 g, 6.28 mmol), DMAP (192 mg, 1.57 mmol) and (S)-borneol (484.38 mg, 3.14 mmol) in anhydrous dichloromethane (30 mL) was stirred at room temperature for 4 h. The reaction mixture was concentrated under reduced pressure and the residue was purified by flash chromatography (SiO₂: ethyl acetate/hexane 0-20%) to afford L44-1 (542 mg, 55%) as slightly yellow oil. ¹H NMR (300 MHz, CDCl₃): o ppm 4.86 (d, J=8.0 Hz, 1H), 4.07 (t, J=7.0 Hz, 2H), 2.40-2.1 (m, 8H), 1.96-1.87 (m, 1H), 1.79-1.46 (m, 12H), 1.39-1.12 (m, 33H), 1.05-0.83 (m, 12H), 0.81 (s, 3H); MS (CI): m/z [M+H]⁺ 634.12.

### Synthesis of 1-(3-pentyloctyl) 17-((1S,2R,4S)-1,7,7-trimethylbicyclo[2.2.1]heptan-2-yl) 9-hydroxyheptadecanedioate (L44-2)

To a mixture of L44-1 (540 mg, 0.853 mmol) in anhydrous THF (10 mL) and anhydrous MeOH (10 mL) was added sodium borohydride (42 mg, 1.1 mmol) at 0° C. The resulting mixture was then stirred at room temperature for 2 h. The reaction was quenched with HCl (1 M), and all the volatile components were reduced under vacuum. The residue was dissolved in diethyl ether and washed with H₂O and brine. The organic phase was dried over Na₂SO₄ and reduced under vacuum to obtain crude material which was purified by silica gel column chromatography (ethyl acetate/hexane 0-20%) to get Compound L44-2 (490 mg, 90.5%) as slightly yellow oil. ¹H NMR (300 MHz, CDCl₃): δ ppm 4.88 (d, J=8.0 Hz, 1H), 4.07 (t, J=7.0 Hz, 2H), 3.56 (s, 1H), 2.34-2.21 (m, 6H), 1.98-1.89 (m, 1H), 1.79-1.51 (m, 9H), 1.49-1.18 (m, 36H), 0.96-0.86 (m, 12H), 0.82 (s, 3H); MS (CI): m/z [M+H]⁺ 636.12.

### Synthesis of 1-(3-pentyloctyl) 17-((1S,2R,4S)-1,7,7-trimethylbicyclo[2.2.1]heptan-2-yl) 9-((4-(dimethylamino)butanoyl)oxy)heptadecanedioate (Compound C56)

A mixture of dimethylamino butanoic acid (155.2 mg, 0.93 mmol), EDC (592 mg, 3.09 mmol) and DMAP (94.3 mg, 0.77 mmol) in anhydrous dichloromethane (10 mL) was stirred at room temperature for 10 min. A solution of L44-2 (490 mg, 0.77 mmol) in anhydrous dichloromethane (5 mL) was added in and the resultant mixture was stirred at room temperature for 4 h. The reaction mixture was concentrated under reduced pressure and the residue was purified by flash chromatography (the flash column was equilibrated with 10% triethylamine in hexane before use, eluent: ethyl acetate/hexane 0-50%) to give Compound C56 (551 mg, 96%). ¹H NMR (300 MHz, CDCl₃): δ ppm 4.88 (d, J=8.0 Hz, 1H), 4.07 (t, J=7.0 Hz, 2H), 2.44-2.20 (m, 16H), 1.98-1.23 (m, 52H), 0.90-0.80 (m, 15H); MS (CI): m/z [M+H]⁺ 749.21 Analytical HPLC column: Agela Durashell C18, 3 μm (Catalog No. DC930505-0), 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: 96.93%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.1 min, purity: >99%.

### Synthesis of Compound C57

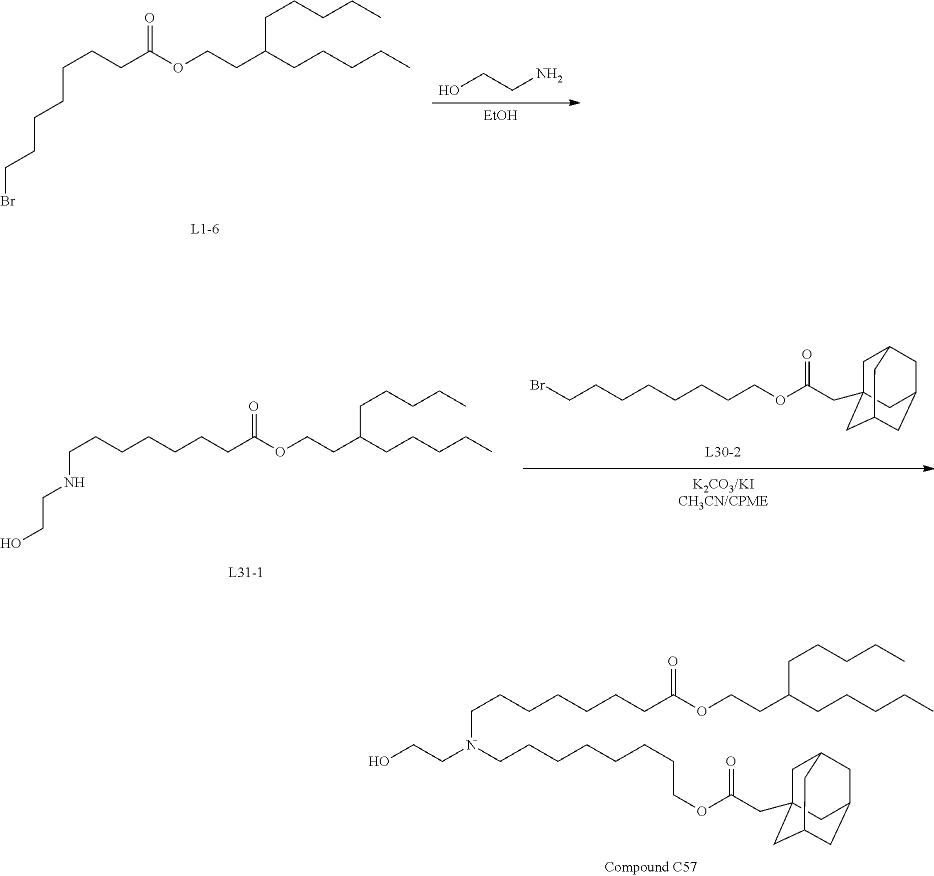

### Synthesis of 3-pentyloctyl 8-((2-hydroxyethyl)amino)octanoate (L31-1)

To a mixture of 3-pentyloctyl 8-bromooctanoate L1-6 (2.55 g, 6.29 mmol) in ethanol (40 mL) was added 2-aminoethanol (3.83 g, 62.9 mmol) and the reaction mixture was heated to 65-70° C. and stirred overnight. The reaction mixture was concentrated, and DCM (150 mL) was added to the residue. After washed with water and brine, the organic phase was dried over Na₂SO₄ and concentrated under reduced pressure to give crude product, which was purified by flash chromatography (SiO₂: 0 to 10% MeOH in DCM (1% NH₄OH) gradient) to yield L31-1 as colorless oil (1.55 g, 64%). ¹H-NMR (300 MHz, CDCl₃) δ 4.07 (t, J=7.14 Hz, 2H), 3.65 (t, J=5.22 Hz, 2H), 2.79 (t, J=5.22 Hz, 2H), 2.62 (t, J=7.14 Hz, 2H), 2.48 (m, 2H), 2.27 (t, J=7.44 Hz, 2H), 1.48-1.6 (m, 5H), 1.17-1.31 (m, 24H) 0.87 (t, J=6.33 Hz, 6H); CIMS m/z [M+H]⁺ 386.3.

### Synthesis of 3-pentyloctyl 8-((8-(2-((3r,5r,7r)-adamantan-1-yl)acetoxy)octyl)(2-hydroxyethyl) amino)octanoate (Compound C57)

To a solution of L31-1 (446 Mg, 1.08 mmol) and L30-2 in AcCN/CPME (1:1, 10 mL) under nitrogen was added K₂CO₃ (447 mg, 3.24 mmol) and KI (90 mg, 0.54 mmol). The reaction mixture was heated at 80° C. overnight under nitrogen atmosphere. After cooling to room temperature, the reaction mixture was filtered through celite and the celite cake was washed with acetonitrile. The filtrate was concentrated to give crude product, which was purified by flash chromatography (SiO₂: 0 to 10% MeOH in DCM (1% NH₄OH) gradient) to yield C₅₇ as colorless oil (505 mg, 68%). ¹H-NMR (300 MHz, CDCl₃) δ 4.05 (m, 4H), 3.62 (m, 2H), 2.69 (m, 2H), 2.56 (m, 4H), 2.27 (t, J=7.4 Hz, 2H), 2.05 (s, 2H), 1.96 (m, 3H), 1.55-1.63 (m, 22H), 1.24-1.38 (m, 32H), 0.87 (m, J=7.14 Hz, 6H); CIMS m/z [M+H]⁺ 690.5. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=9.28 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=9.37 min, purity: 94.69%.

### Synthesis of Compound C58

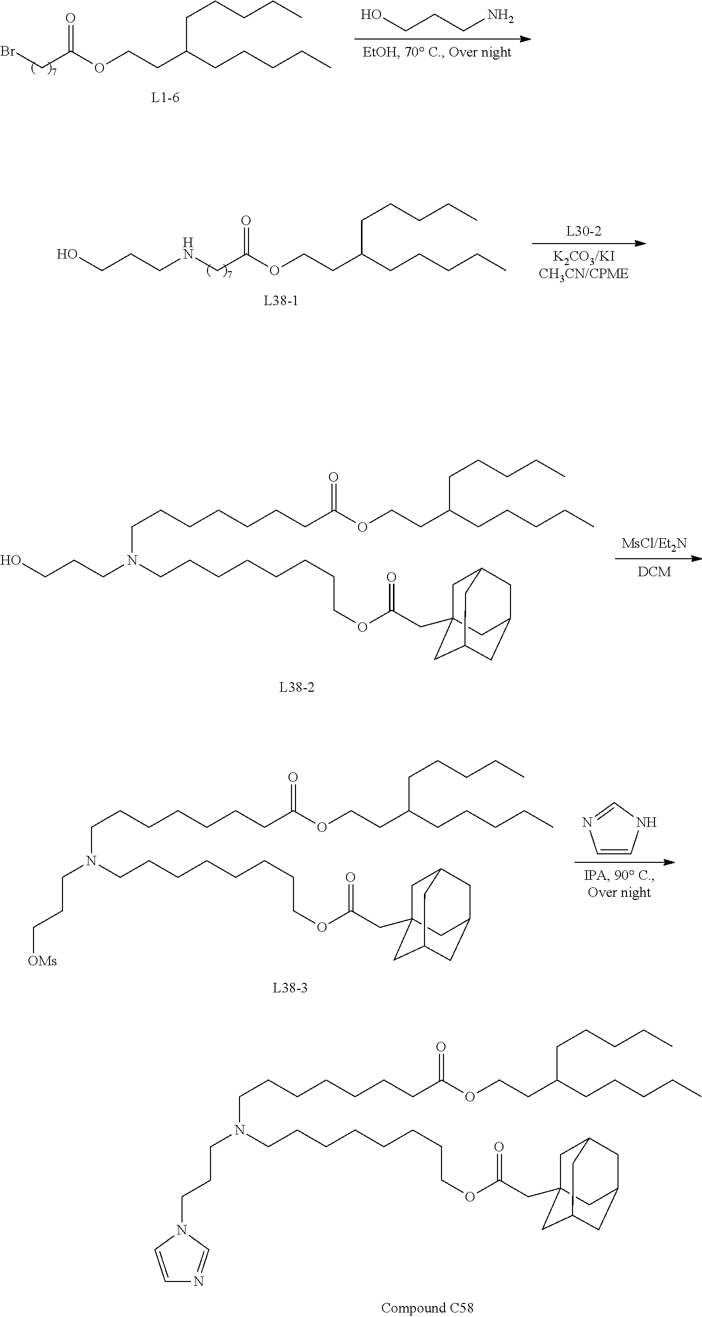

### Synthesis of 3-pentyloctyl 8-((3-hydroxpropyl)amino)octanoate (L38-1)

To a solution of L1-6 (1.35 g, 3.33 mmol) in ethanol (25 mL) was added 3-aminopropanol (2.49 g, 33.3 mmol). The reaction mixture was heated to 70° C. and stirred overnight. The reaction mixture was concentrated, and DCM (100 mL) was added to the residue. After washed with water (50 mL), brine (50 mL) and dried over Na₂SO₄, the organic phase was concentrated under reduced pressure to give crude product, which was purified by flash chromatography (SiO₂: 0 to 10% MeOH in DCM (1% NH₄OH) gradient) to yield L38-1 as colorless oil (870 mg, 65%). ¹H-NMR (300 MHz, CDCl₃) δ 4.06 (t, J=6.87 Hz, 2H), 3.8 (t, J=5.4 Hz, 2H), 2.87 (t, J=5.79 Hz, 2H), 2.58 (t, J=7.14 Hz, 2H), 2.27 (t, J=7.41 Hz, 2H), 1.54-1.69 (m, 7H), 1.24-1.29 (m, 26H) 0.87 (t, J=6.6 Hz, 6H); CIMS m/z [M+H]⁺ 400.4.

### Synthesis of 3-pentyloctyl 8-((8-(2-((3r,5r,7r)-adamantan-1-yl)acetoxy)octyl)(3-hydroxy propyl)amino) octanoate (L38-2)

To a solution of L38-1 (870 Mg, 2.18 mmol) and L30-2 (1.0 g, 2.61 mmol) in ACN/CPME (1:1, 20 mL) under nitrogen was added K₂CO₃ (900 mg, 6.54 mmol) and KI (180 mg, 1.09 mmol). The reaction mixture was heated at 80° C. overnight under nitrogen atmosphere. After cooling to room temperature, the reaction mixture was filtered through celite and the celite cake was washed with acetonitrile. The filtrate was concentrated to give crude product, which was purified by flash chromatography (SiO₂: 0 to 6% MeOH in DCM gradient) to yield L38-2 as colorless oil (1.08 g, 70%). ¹H-NMR (300 MHz, CDCl₃) δ 4.05 (m, 4H), 3.79 (m, 2H), 2.67 (m, 2H), 2.43 (m, 4H), 2.27 (t, J=7.4 Hz, 2H), 2.05 (s, 2H), 1.96 (m, 2H), 1.55-1.68 (m, 20H), 1.5 (m, 3H), 1.24 (m, 34H), 0.87 (t, J=6.6 Hz, 6H); CIMS m/z [M+H]⁺ 704.6.

### Synthesis of 3-pentyloctyl 8-((8-(2-((3r,5r,7r)-adamantan-1-yl)acetoxy)octyl)(3-((methylsulfonyl)oxy) propyl)amino)octanoate (L38-3)

To a solution of L38-2 (1.08 g, 1.53 mmol) in anhydrous DCM (15 mL) was added methanesulfonyl chloride (141 μL, 1.83 mmol) and triethylamine (385 mL, 3.82 mmol)) at room temperature. The solution was stirred at room temperature for 1 hour. Reaction mixture was diluted with DCM (75 mL) and washed with water (2×50 mL) and brine (50 mL). The organic layer was dried over Na₂SO₄, and solvent was reduced under vacuum. The crude product L38-3 was dried under high vacuum for 1 hour and used for next step without further purification and characterization.

### Synthesis of 3-pentyloctyl 8-((3-(1H-imidazol-1-yl)propyl)(8-(2-((3r,5r,7r)-adamantan-1-yl)acetoxy) octyl)amino)octanoate (Compound C58)

To a solution of crude L38-3 in isopropanol (15 mL) was added imidazole (2.0 g, 30.6 mmol). The reaction mixture was stirred at 90° C. under nitrogen overnight. The solvent was reduced under vacuum, and the residue was dissolved in DCM (75 mL) and washed with water (2×50 mL). Organic layer was dried over Na₂SO₄, and solvent was reduced under vacuum. Crude product was purified by flash chromatography (SiO₂: 0 to 5% MeOH in DCM gradient) to yield Compound C58 as light-yellow oil (870 mg, 75%). ¹H-NMR (300 MHz, CDCl₃): 7.45 (s, 1H), 7.04 (s, 1H), 6.9 (s, 1H), 6 4.03 (m, 6H), 2.32 (m, 8H), 2.05 (s, 2H), 1.96 (m, 3H), 1.85 (m, 1H), 1.6 (m, 20H), 1.28 (m, 34H), 0.87 (m, 6H); CIMS m/z [M+H]⁺ 754.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=5.78 min, purity: >99.59%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=6.52 min, purity: 95.69%.

### Synthesis of Compound C59 PGP-865 C₃

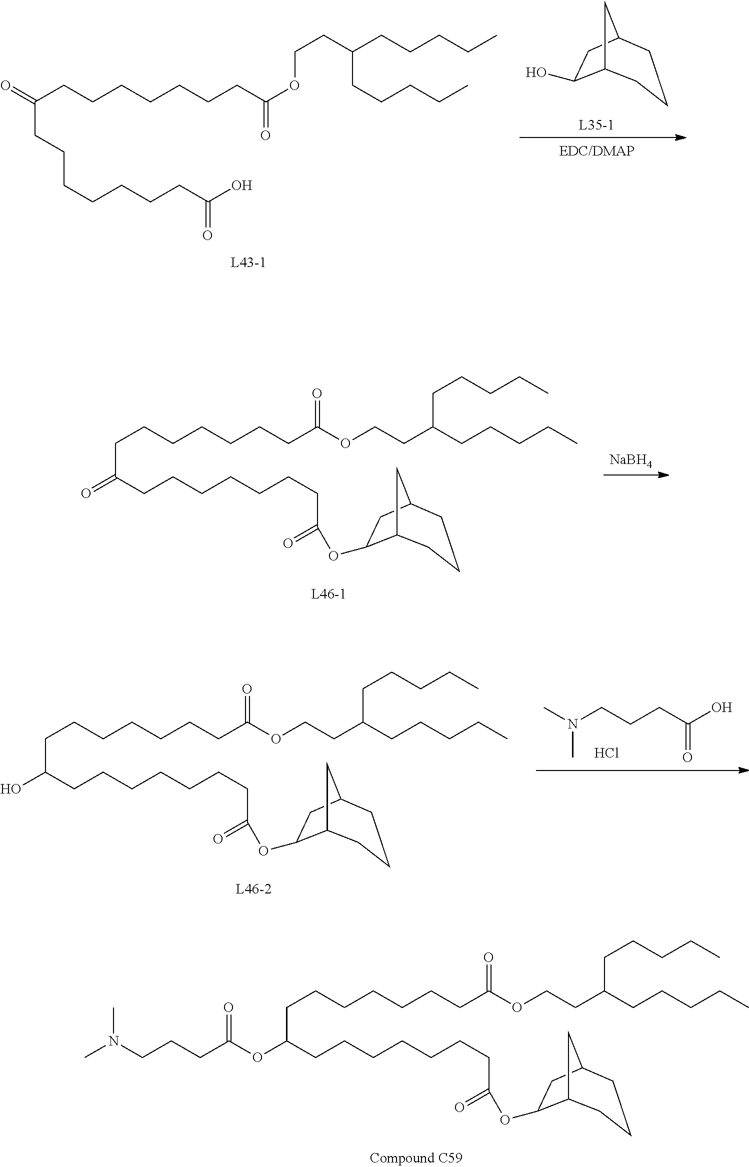

### Synthesis of 1-((1R,5S)-bicyclo[3.2.1]octan-6-yl) 17-(3-pentyloctyl) 9-oxoheptadecanedioate (L46-1)

A mixture of L43-1 (700 mg, 1.41 mmol), EDC (1.08 g, 5.65 mmol), DMAP (173 mg, 1.41 mmol) and L35-1 (436 mg, 2.82 mmol) in anhydrous dichloromethane (25 mL) was stirred at room temperature for 4 h. The reaction mixture was concentrated under reduced pressure and the residue was purified by flash chromatography (SiO₂: ethyl acetate/hexane 0-20%) to afford L46-1 (510 mg, 60%) as colorless oil. ¹H-NMR (300 MHz, CDCl₃) δ 4.95 (m, 1H), 4.07 (t, 2H), 2.39-2.23 (m, 9H), 2.08-2.01 (m, 2H), 1.80-1.18 (m, 48H), 0.88 (t, 6H).

### Synthesis of 1-((1R,5S)-bicyclo[3.2.1]octan-6-yl) 17-(3-pentyloctyl) 9-hydroxyhepta decanedioate (L46-2)

To a mixture of L46-1 (510 mg, 0.805 mmol) in anhydrous THF (10 mL) and anhydrous MeOH (10 mL) was added sodium borohydride (37 mg, 1.0 mmol) at 0° C. The resulting mixture was then stirred at room temperature for 2 h. The reaction was quenched with HCl (1 M), and all the volatile components were reduced under vacuum. The residue was dissolved in diethyl ether and washed with H₂O and brine. The organic phase was dried over Na₂SO₄ and reduced under vacuum to obtain crude material which was purified by silica gel column chromatography (ethyl acetate/hexane 0-20%) to get Compound L46-2 (320 mg, 63%) as light brown oil. ¹H-NMR (300 MHz, CDCl₃) δ 4.95 (m, 1H), 4.07 (t, 2H), 3.57 (bs, 1H), 2.30-2.20 (m, 5H), 2.08-1.99 (m, 2H), 1.80-1.18 (m, 48H), 0.88 (t, 6H).

### Synthesis of 1-((1R,5S)-bicyclo[3.2.1]octan-6-yl) 17-(3-pentyloctyl) 9-((4-(dimethylamino) butanoyl) oxy)heptadecanedioate (Compound C59)

A mixture of dimethylamino butanoic acid (81 mg, 0.5 mmol), EDC (319 mg, 1.66 mmol) and DMAP (51.2 mg, 0.42 mmol) in anhydrous dichloromethane (8 mL) was stirred at room temperature for 10 min. A solution of L46-2 (300 mg, 0.42 mmol) in anhydrous dichloromethane (5 mL) was added and the resultant mixture was stirred at room temperature for 4 h. The reaction mixture was concentrated under reduced pressure and the residue was purified by flash chromatography (the flash column was equilibrated with 10% triethylamine in hexane before use, eluent: ethyl acetate/hexane 0-50%) to give Compound C₅₉ (620 mg, 62%) as light brown oil. ¹H-NMR (300 MHz, CDCl₃) δ 4.96-4.82 (m, 2H), 4.07 (t, 2H), 2.32-2.22 (m, 15H), 2.08-1.99 (m, 2H), 1.82-1.10 (m, 62H), 0.87 (t, 6H); CIMS m/z 720.6 [M+1]; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.75 min, purity: >99%. UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=11.57 min, purity: 90.45%.

### Synthesis of Compound C60

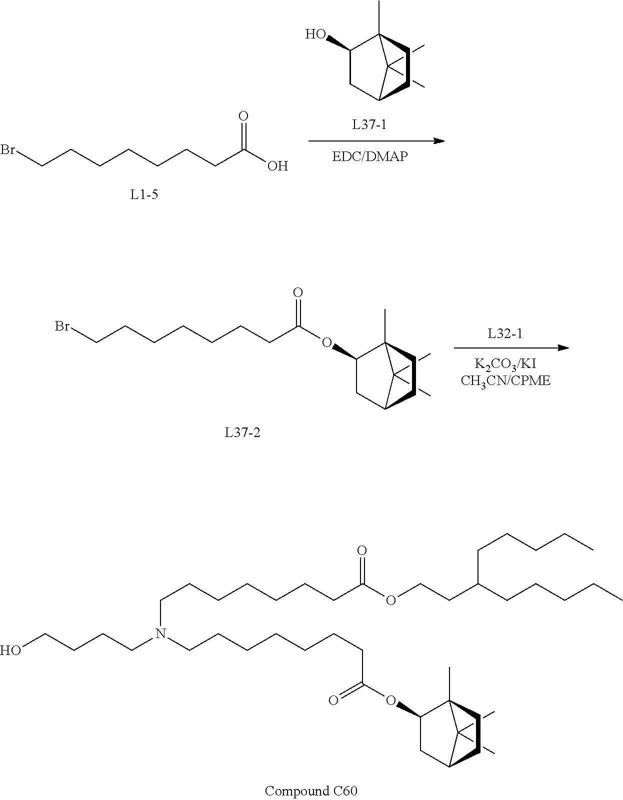

### Synthesis of 2-(9-bromononyl)-1,7,7-trimethylbicyclo[2.2.1]heptane (L37-2)

To a DCM (30 ml) solution of L37-1 (2.0 g, 12.98 mmol, 1.0 eq) and bromo-acid L1-5 (3.74 g, 16.88 mmol, 1.3 eq) was added EDC (7.44 g, 38.96 mmol, 3.0 eq) and DMAP (792 mg, 6.44 mmol). The solution was stirred for 3 h at room temperature and then concentrated under reduced pressure. The crude was purified by flash chromatography (solvent: 0-10% EA in Hexane) to give L37-2 as color less oil (3.21 g, 68.7%). ¹H-NMR (300 MHz, CHCl₃) δ: 4.85-4.90 (dd, 1H), 3.37-3.39 (t, 1H), 2.28-2.30 (t, 2H), 1.35 to 1.84 (m, 6H), 1.24-1.35 (m, 7H), 0.81 to 0.89 (3S, 9H).

### Synthesis of 3-pentyloctyl 8-((4-hydroxybutyl) (8-oxo-8-(((1S,2R,4S)-1,7,7-trimethylbicyclo [2.2.1]heptan-2-yl) oxy) octyl) amino) octanoate (Compound 60)

A mixture of L32-1 (750 mg, 1.82 mmol, 1.0 eq), L37-2 (750 mg, 2.3 mmol), potassium carbonate (150 mg, 0.9 mmol) and KI (150 mg, 0.9 mmol) with CPME (15 ml) and ACN (15 ml) was heated to 80° C. for 18 h. After cooling to room temperature, the reaction mixture was filtered through celite. The celite plug was washed with ACN (150 ml). The filtrate was concentrated. The crude material was purified by column chromatography (Solvent: 0-10% MeOH in DCM) to give Compound C60 as a colorless oil (390 mg, 32.6%). ¹HNMR (CDCl₃) δ: 0.8091-0.8896 (m, 15H), 1.239-1.299 (m, 22H), 1.543-1.567 (m, 1H), 1.540-1.550 (m, 9H), 2.241-2.320 (q, 4H), 2.500-2.650 (bs, 4H), 3.567 (s, 1H), 4.038-4.086 (t, IH), 4.89-4.990 (d, 1H); CIMS m/z [M+H]⁺ 692.7; CIMS m/z [M+H]⁺ 634.1. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=7.10 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=8.92 min, purity: 97.6%.

### Synthesis of Compound C62

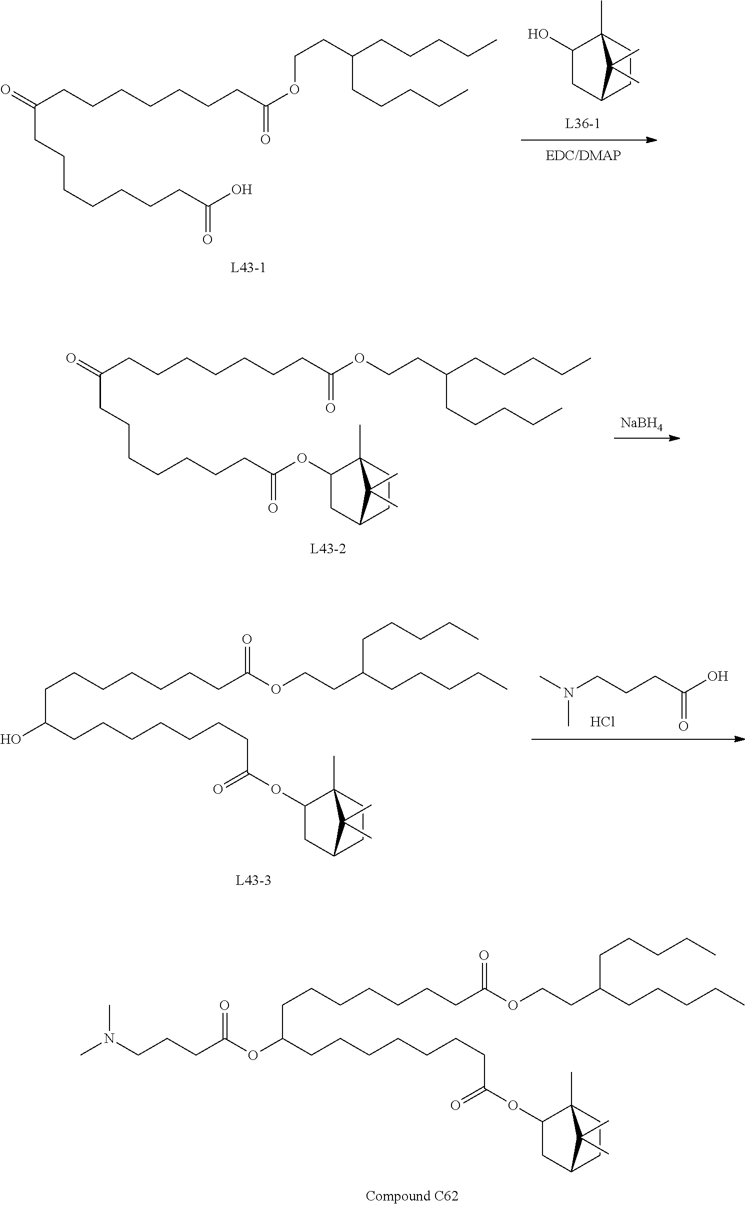

### Synthesis of 1-(3-pentyloctyl) 17-(1,7,7-trimethylbicyclo[2.2.1]heptan-2-yl) 9-oxohepta decanedioate (L43-2)

A mixture of L43-1 (230 mg, 0.46 mmol), EDC (360 mg, 1.88 mmol), DMAP (58 mg, 0.46 mmol) and (R)-borneol L36-1 (146 mg, 0.94 mmol) in anhydrous dichloromethane (12 mL) was stirred at room temperature for 4 h. The reaction mixture was concentrated under reduced pressure and the residue was purified by flash chromatography (SiO₂: ethyl acetate/hexane 0-20%) to afford L43-2 (220 mg, 75%) as colorless oil. ¹H-NMR (300 MHz, CDCl₃) δ 4.89-4.85 (m, 1H), 4.07 (t, 2H), 2.33-2.25 (q, 5H), 2.20-2.05 (m, 2H), 1.68-1.57 (m, 9H), 1.40-1.18 (m, 38H), 0.88 (m, 12H), 0.81 (s, 3H).

### Synthesis of 1-(3-pentyloctyl) 17-(1,7,7-trimethylbicyclo[2.2.1]heptan-2-yl) 9-hydroxyheptadecanedioate (L43-3)

To a mixture of L43-2 (220 mg, 0.34 mmol) in anhydrous THF (4 mL) and anhydrous MeOH (4 mL) was added sodium borohydride (17 mg, 0.44 mmol) at 0° C. The resulting mixture was then stirred at room temperature for 2 h. The reaction was quenched with HCl (1 M), and all the volatile components were reduced under vacuum. The residue was dissolved in diethyl ether and washed with H₂O and brine. The organic phase was dried over Na₂SO₄ and reduced under vacuum to obtain crude material which was purified by silica gel column chromatography (ethyl acetate/hexane 0-20%) to get Compound L43-3 (135 mg, 61%) as colorless oil. ¹H-NMR (300 MHz, CDCl₃) δ 4.89-4.85 (m, 1H), 4.07 (t, 2H), 3.55 (s, 1H), 2.33-2.25 (m, 4H), 1.65-1.52 (m, 4H), 1.41-1.18 (m, 38H), 0.88 (m, 11H), 0.82 (s, 3H).

### Synthesis of 1-(3-pentyloctyl) 17-(1,7,7-trimethylbicyclo[2.2.1]heptan-2-yl) 9-((4-(dimethylamino)butanoyl)oxy)heptadecanedioate (Compound C62)

A mixture of dimethylamino butanoic acid (36 mg, 0.22 mmol), EDC (138 mg, 0.73 mmol) and DMAP (22 mg, 0.18 mmol) in anhydrous dichloromethane (5 mL) was stirred at room temperature for 10 min. A solution of L43-3 (135 mg, 0.18 mmol) in anhydrous dichloromethane (5 mL) was added and the resultant mixture was stirred at room temperature for 4 h. The reaction mixture was concentrated under reduced pressure and the residue was purified by flash chromatography (the flash column was equilibrated with 10% triethylamine in hexane before use, eluent: ethyl acetate/hexane 0-50%) to give Compound C62 (120 mg, 75%) as colorless oil. ¹H-NMR (300 MHz, CDCl₃) δ 4.89-4.85 (m, 2H), 4.07 (t, 2H), 2.32-2.24 (m, 16H), 1.95-1.48 (m, 15H), 1.27-1.08 (m, 35H), 0.88 (m, 12H), 0.82 (s, 3H); CIMS m/z 748.7 [M+1]; Analytical HPLC column: Agela Durashell C18, 3 m (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.01 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=11.64 min, purity: 98.14%.

### Synthesis of Compound C63

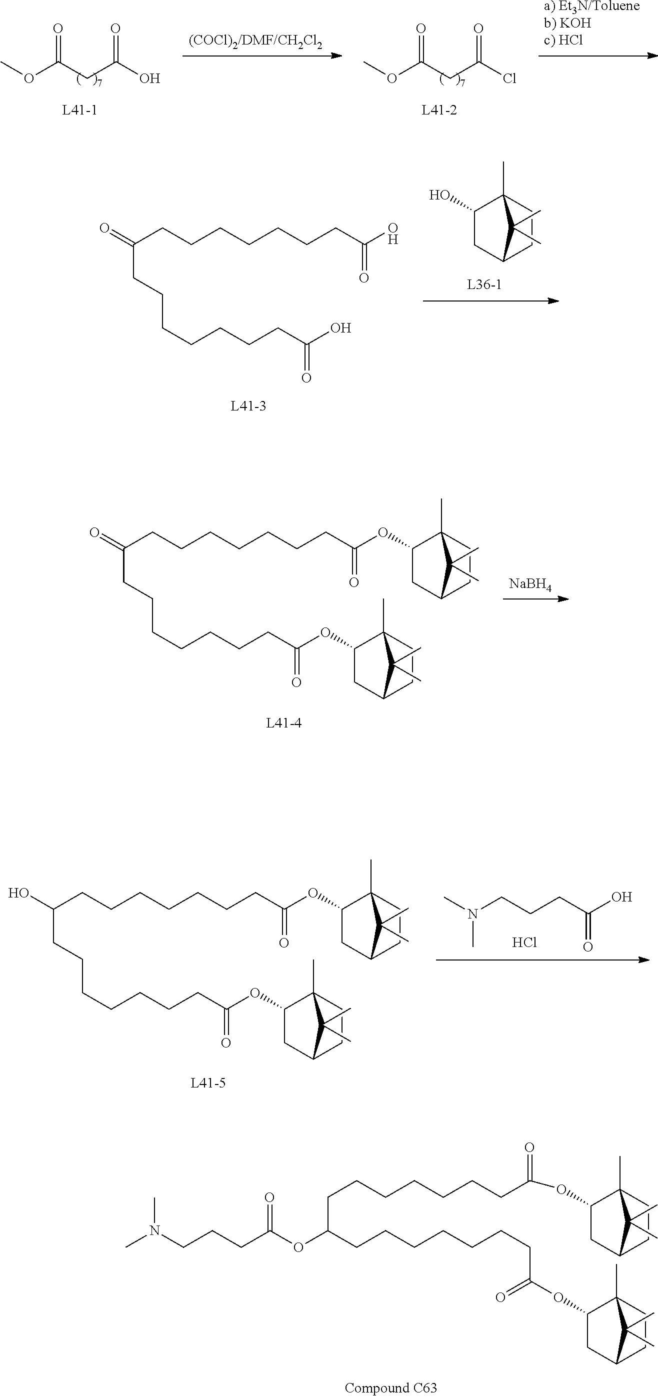

### Synthesis of methyl 9-chloro-9-oxononanoate (L41-2)

To a solution of L41-1 (12 g, 59 mmol) in anhydrous DCM (40 mL) was added anhydrous DMF (1 mL). With ice bath cooling, a solution of oxalyl chloride (8.2 g, 65 mmol) in anhydrous DCM (10 mL) was dropped in under nitrogen with stirring. The resulting mixture was then stirred at room temperature under nitrogen overnight. The reaction mixture was concentrated and co-evaporated with anhydrous toluene to give L41-2 as colorless oil (11.6 g, 97%). ¹H-NMR (300 MHz, CDCl₃) δ 3.63 (s, 3H), 2.85 (t, J=7.2 Hz, 2H), 2.27 (t, J=7.5 Hz, 2H), 1.77-1.48 (m, 4H), 1.41-1.19 (m, 6H).

### Synthesis of 9-oxoheptadecanedioic acid (L41-3)

To an ice bath cooled solution of L41-2 (11 g, 50 mmol) in anhydrous toluene (85 mL) was added triethylamine (5 g, 50 mmol) in 10 min with stirring while keeping the reaction temperature below 25° C. After addition finished, the reaction mixture temperature was brought to 35-40° C. during 15-20 min with a warm water bath. After the temperature reached to 40° C., the water bath was removed, and the reaction mixture was stirred for 1 h. It was then filtered through a short pad of celite and the celite cake was rinsed with toluene (25 mL). The combined filtrates were reduced under vacuum to give an oil residue which was mixed with 2N aq. KOH (42 mL). The mixture was refluxed for 6 h and then cooled in ice-bath. The aqueous layer was washed with ether (35 mL×3) and acidified with concentrated HCl to pH 4. After cooling the mixture in ice bath for 1 h, the precipitates were filtered, washed with ice cold water and dried to yield L41-3 as an off-white solid (6.0 g, 71%). ¹H-NMR (300 MHz, DMSO-d6) δ 2.38 (t, J=7.1 Hz, 4H), 2.18 (t, J=7.4 Hz, 4H), 1.53-1.35 (m, 8H), 1.33-1.02 (m, 12H).

### Synthesis of bis((1R,2S,4R)-1,7,7-trimethylbicyclo[2.2.1]heptan-2-yl) 9-oxohepta decanedioate (L41-4)

To a solution of L41-3 (950 mg, I eq) and L36-1 (978 mg, 2.1 eq) in dichloromethane (19 mL) was added EDC (2.3 g, 4 eq) and DMAP (369 mg, 1 eq). The reaction mixture was stirred under nitrogen for 31 hours until TLC (20% EA in hexane, R_{f}=0.9) showed completion of the reaction. Solvent was removed under vacuum to obtain crude material which was purified by silica gel column chromatography using 0-40% EA in hexane as eluent to afford L41-4 (1.03 g, 57%). ¹H-NMR (300 MHz, CDCl₃) δ 4.86 (tt, 2H), 2.42-2.24 (m, 10H), 1.98-1.84 (m, 2H), 1.82-1.46 (m, 12H), 1.38-1.14 (m, 16H), 0.89 (s, 6H), 0.87 (s, 6H), 0.81 (s, 6H); CIMS m/z [M+H]⁺ 587.5.

### Synthesis of ethyl bis((1R,2S,4R)-1,7,7-trimethylbicyclo[2.2.1]heptan-2-yl) 9-hydroxyhepta decanedioate (L41-5)

To a solution of L41-4 (1.03 g, 1 eq) in THF (6 mL) and methanol (3 mL) was added sodium borohydride (70.8 mg, 1 eq) at room temperature. The reaction mixture was stirred under nitrogen atmosphere until TLC (20% EA in hexane, R_{f}=0.5) showed completion of the reaction. The reaction was quenched with HCl (1 M), and all the volatile components were reduced under vacuum. The residue was dissolved in diethyl ether and washed with H2O and brine. The organic phase was dried over Na₂SO₄ and reduced under vacuum to obtain crude material which was purified by silica gel column using 0-50% EA in hexane as eluent to yield L41-5 (825 mg, 80%). ¹H-NMR (300 MHz, CDCl₃) δ 4.86 (tt, 2H), 3.55 (bs, 1H), 2.42-2.24 (m, 10H), 1.98-1.84 (m, 2H), 1.82-1.46 (m, 12H), 1.38-1.14 (m, 16H), 0.89 (s, 6H), 0.87 (s, 6H), 0.81 (s, 6H); CIMS m/z [M+H]⁺ 589.5.

### Synthesis of bis((1R,2S,4R)-1,7,7-trimethylbicyclo[2.2.1]heptan-2-yl) 9-((4-(dimethylamino)butanoyl)oxy)heptadecanedioate (Compound C63)

To a solution of L41-2 (825 mg, I eq), 4-(dimethylamino)butanoic acid (304 mg, 1.2 eq), 4-dimethylaminopyridine (34 mg, 0.2 eq) and N, N-diisopropylethylamine (0.5 mL, 2 eq) in DCM (7.5 mL) was added EDC (358 mg, 1.33 eq). The mixture was purged with nitrogen and stirred for 16 hours. The reaction was diluted with DCM, washed with aq. NaHCO₃ and brine. The organic phase was dried over Na₂SO₄ and reduced under vacuum to obtain crude product which was purified by silica column chromatography using 0-10% MeOH in DCM as eluent to afford Compound C63 (0.87 g, 89%). ¹H-NMR (300 MHz, CDCl₃) δ 4.86 (m, 3H), 2.38-2.22 (m, 10H), 2.21 (s, 6H), 1.96-1.89 (m, 2H), 1.80-1.70 (m, 4H), 1.67 (t, 2H), 1.63-1.58 (m, 4H), 1.50-1.48 (m, 4H), 1.32-1.18 (m, 20H), 0.96 (d, 1H), 0.93 (d, 1H), 0.90 (s, 6H), 0.87 (s, 6H), 0.81 (s, 6H); CIMS m/z [M+H]⁺ 702.5. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 15 min, flow rate: 1 mL/min, column temperature: 20±2° C., detector: ELSD, t_{R}=5.82 min, purity: >99.9%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 70% to 100% in 20 min, flow rate: 1 mL/min, column temperature: 20±2° C., detector: CAD, t_{R}=8.75 min, purity: >99.9%.

### Synthesis of Compound C64

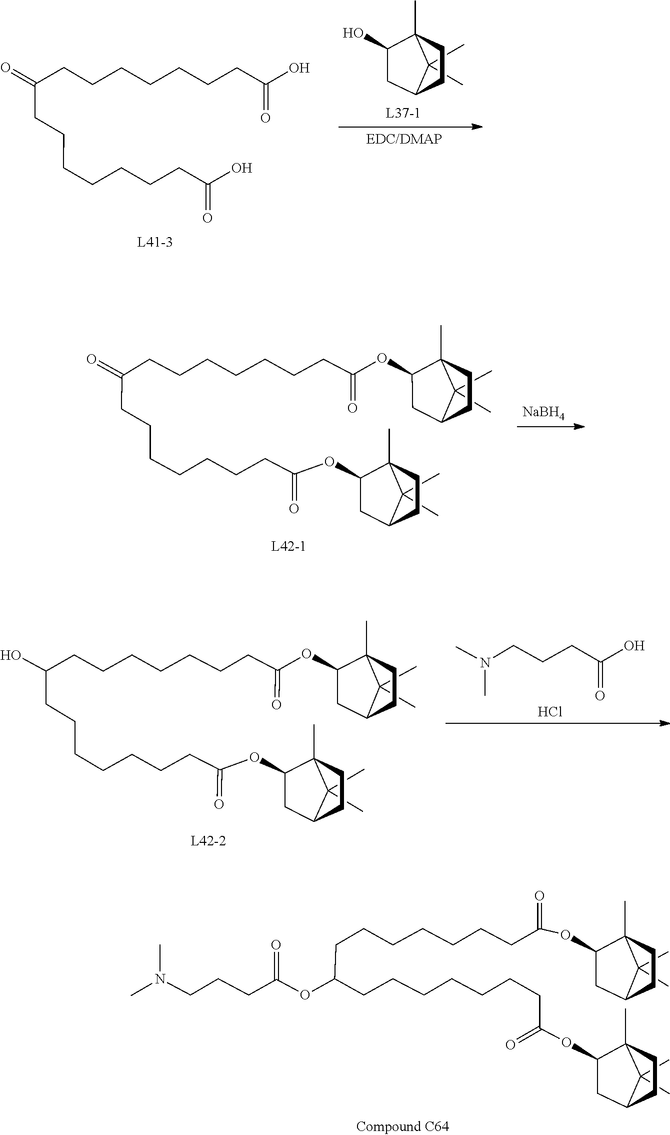

### Synthesis of bis((1S,2R,4S)-1,7,7-trimethylbicyclo[2.2.1]heptan-2-yl) 9-oxoheptadecanedioate (L42-1)

To a solution of L41-3 (1.0 g, 1 eq) and L37-1 (1.03 g, 2.1 eq) in dichloromethane (20 mL) was added EDC (2.44 g, 4 eq) and 4-dimethylaminopyridine (3.88 g, 1 eq). The reaction mixture was stirred under N₂ for 31 hours until TLC (20% EA in hexane, R_{f}=0.9) showed completion of the reaction. Solvent was removed under vacuum to obtain crude material which was then purified by silica gel column using 0-70% EA in hexane as eluent to afford L42-1 (1.51 g, 80%). ¹H-NMR (300 MHz, CDCl₃) δ 4.86 (tt, 2H), 2.42-2.24 (m, 10H), 1.98-1.84 (m, 2H), 1.82-1.46 (m, 12H), 1.38-1.14 (m, 16H), 0.89 (s, 6H), 0.87 (s, 6H), 0.81 (s, 6H); CIMS m/z [M+H]⁺ 587.5.

### Synthesis of bis((1S,2R,4S)-1,7,7-trimethylbicyclo[2.2.1]heptan-2-yl) 9-hydroxyheptadecanedioate (L42-2)

To a solution of L42-1 (1.51 g, 1 eq) in THF (9 mL) and methanol (4.5 mL) was added sodium borohydride (103 mg, 1 eq). The reaction mixture was stirred under nitrogen atmosphere for 16 hours. The reaction was quenched with HCl (1 M), and all the volatile components were reduced under vacuum. The residue was dissolved in diethyl ether and washed with H₂O and brine. The organic phase was dried over Na₂SO₄ and reduced under vacuum to obtain crude material which was purified by silica gel column using 0-50% EA in hexane as eluent to yield L42-2 (1.09 g, 72%). ¹H-NMR (300 MHz, CDCl₃) δ 4.86 (tt, 2H), 3.55 (bs, 1H), 2.42-2.24 (m, 10H), 1.98-1.84 (m, 2H), 1.82-1.46 (m, 12H), 1.38-1.14 (m, 16H), 0.89 (s, 6H), 0.87 (s, 6H), 0.81 (s, 6H); CIMS m/z [M+H]⁺ 589.5.

### Synthesis of bis((1S,2R,4S)-1,7,7-trimethylbicyclo[2.2.1]heptan-2-yl) 9-((4-(dimethylamino)butanoyl)oxy)heptadecanedioate (Compound C64)

To a mixture of L42-2 (1090 mg, 1 eq), 4-(dimethylamino)butanoic acid (402 mg, 1.3 eq), 4-dimethylaminopyridine (45 mg, 0.2 eq), N, N-diisopropylethylamine (0.64 mL, 2 eq) in DCM (10 mL) was added EDC (473 mg, 1.33 eq). The mixture was purged with nitrogen and stirred for 16 hours. The reaction mixture was then diluted with DCM and washed with NaHCO₃ and brine. The organic phase was dried over Na₂SO₄ and reduced under vacuum to obtain crude product which was purified by silica gel using 0-90% EA with 1% triethylamine in hexane as eluent to yield Compound C64 (0.773 g, 59%). ¹H-NMR (300 MHz, CDCl₃) δ 4.86 (m, 3H), 2.38-2.22 (m, 10H), 2.21 (s, 6H), 1.96-1.89 (m, 2H), 1.80-1.70 (m, 4H), 1.67 (t, 2H), 1.63-1.58 (m, 4H), 1.50-1.48 (m, 4H), 1.32-1.18 (m, 20H), 0.96 (d, 1H), 0.93 (d, 1H), 0.90 (s, 6H), 0.87 (s, 6H), 0.81 (s, 6H); CIMS m/z [M+H]⁺ 702.5. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 15 min, flow rate: 1 mL/min, column temperature: 20±2° C., detector: ELSD, t_{R}=5.94 min, purity: >99.9%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 70% to 100% in 20 min, flow rate: 1 mL/min, column temperature: 20±2° C., detector: CAD, t_{R}=8.71 min, purity: >99.9%.

### Synthesis of Compound C65

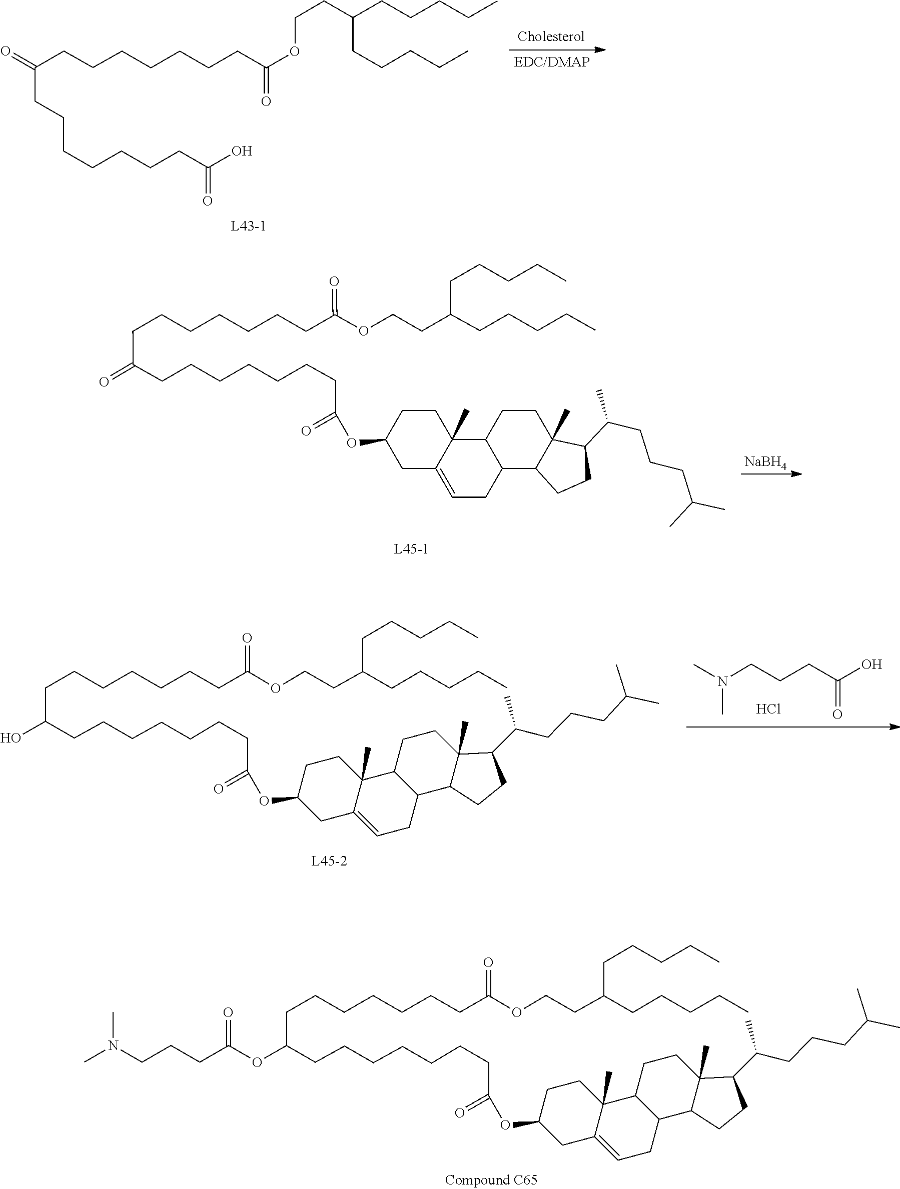

### Synthesis of 1-((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 17-(3-pentyloctyl)-9-oxoheptadecanedioate (L45-1)

A mixture of L43-1 (300 mg, 0.604 mmol), EDC (467.07 mg, 2.415 mmol), DMAP (74 mg, 0.604 mmol) and cholesterol (466.99 mg, 1.207 mmol) in anhydrous dichloromethane (30 mL) was stirred at room temperature for 2 h. The reaction mixture was concentrated under reduced pressure and the residue was purified by flash chromatography (SiO₂: ethyl acetate/hexane 0-20%) to afford Compound L45-1 (301 mg, 29%) as slightly yellow oil. ¹H NMR (300 MHz, CDCl₃): δ ppm 5.35 (d, J=5.0 Hz, 1H), 4.70-4.55 (m, 1H), 4.07 (t, J=6.0 Hz, 2H), 2.43-2.20 (m, 12H), 2.10-1.79 (m, 6H), 1.73-0.81 (m, 75H), 0.67 (s, 3H); MS (CI): m/z [M+H]⁺ 866.44.

### Synthesis of 1-((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 17-(3-pentyloctyl) 9-hydroxyheptadecanedioate (L45-2)

To a mixture of L45-1 (300 mg, 0.604 mmol) in anhydrous THF (10 mL) and anhydrous MeOH (10 mL) was added sodium borohydride (30 mg, 0.785 mmol) at 0° C. The resulting mixture was then stirred at room temperature for 2 h. The reaction was quenched with HCl (1 M), and the volatile components were reduced under vacuum. The residue was dissolved in diethyl ether and washed with H₂O and brine. The organic phase was dried over Na₂SO₄ and reduced under vacuum to obtain crude material which was purified by silica gel column chromatography (ethyl acetate/hexane 0-20%) to get Compound L45-2 (290 mg, 96%) as slightly yellow oil. ¹H NMR (300 MHz, CDCl₃): δ ppm 5.35 (d, J=5.0 Hz, 1H), 4.68-4.54 (m, 1H), 4.07 (t, J=6.0 Hz, 2H), 3.56 (s, 1H), 2.35-2.20 (m, 6H), 2.06-1.76 (m, 6H), 1.71-0.81 (m, 82H), 0.66 (s, 3H); MS (CI): m/z [M+H]⁺ 868.44.

### Synthesis of 1-((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 17-(3-pentyloctyl) 9-((4-(dimethylamino)butanoyl)oxy)heptadecanedioate (Compound C65)

A mixture of dimethylamino butanoic acid (65 mg, 0.387 mmol), EDC (2.475 g, 1.29 mmol), and DMAP (39.5 mg, 0.322 mmol) in anhydrous dichloromethane (30 mL) was stirred at room temperature for 10 min. A solution of L45-2 (280 mg, 0.322 mmol) in anhydrous dichloromethane (5 mL) was added and the resultant mixture was stirred at room temperature for 4 h. The reaction mixture was concentrated under reduced pressure and the residue was purified by flash chromatography (the flash column was equilibrated with 10% triethylamine in hexane before use, eluent: ethyl acetate/hexane 0-50%) to give Compound C₆₅ (200 mg, 53%). ¹H NMR (300 MHz, CDCl₃): δ 5.45 (d, J=5.0 Hz, 1H), 4.88-4.68 (m, 1H), 4.67-4.58 (m, 1H), 4.08 (t, J=6.0 Hz, 2H), 2.33-2.1 (m, 20H), 2.10-1.92 (m, 2H), 1.96-1.76 (m, 7H), 1.62-1.12 (m, 37H), 1.10-0.87 (m, 5H), 0.86-0.83 (m, 14H), 0.66 (s, 3H); MS (CI): m/z [M+H]⁺ 980.60. Analytical HPLC column: Agela Durashell C18, 3 μm (Catalog No. DC930505-0), 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: >98.3%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.1 min, purity: >99%.

### Synthesis of Compound C66

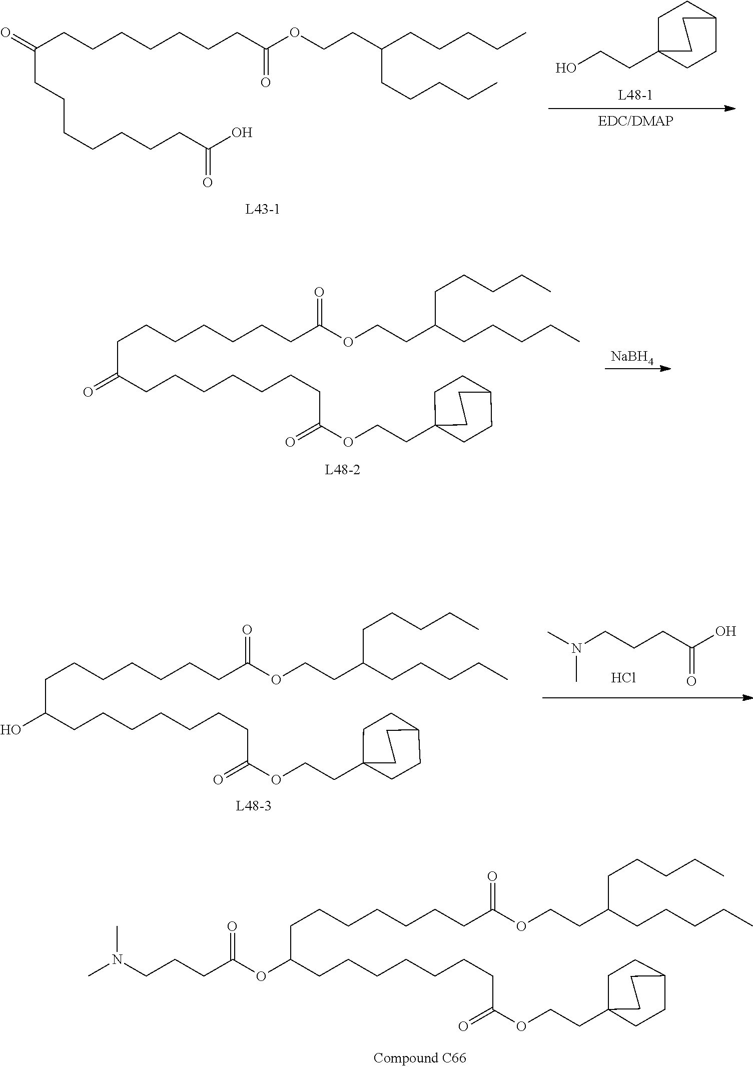

### Synthesis of 1-(2-(bicyclo[2.2.2]octan-1-yl)ethyl) 17-(3-pentyloctyl) 9-oxohepta-decanedioate (L48-2)

A mixture of L43-1 (532 mg, 1.07 mmol), EDC (746 mg, 3.88 mmol), DMAP (119 mg, 0.97 mmol) and L48-1 (150 mg, 0.97 mmol) in anhydrous dichloromethane (10 mL) was stirred at room temperature for 4 h. The reaction mixture was concentrated under reduced pressure and the residue was purified by flash chromatography (SiO₂: ethyl acetate/hexane 0-20%) to afford Compound L48-2 (530 mg, 73%) as slightly yellow oil. ¹H NMR (300 MHz, CDCl₃): δ ppm 4.19-4.02 (m, 4H), 2.41-2.21 (m, 8H), 1.82-1.20 (m, 54H), 0.87 (t, J=7.0 Hz, 6H). MS (CI): m/z [M+H]⁺ 634.2

### Synthesis of 1-(2-(bicyclo[2.2.2]octan-1-yl)ethyl) 17-(3-pentyloctyl) 9-hydroxyhepta decanedioate (L48-3)

To a mixture of L48-2 (520 mg, 0.821 mmol) in anhydrous THF (10 mL) and anhydrous MeOH (10 mL) was added sodium borohydride (41 mg, 1.1 mmol) at 0° C. The resulting mixture was then stirred at room temperature for 2 h. The reaction was quenched with HCl (1 M), and all the volatile components were reduced under vacuum. The residue was dissolved in diethyl ether and washed with H₂O and brine. The organic phase was dried over Na₂SO₄ and reduced under vacuum to obtain crude material which was purified by silica gel column (ethyl acetate/hexane 0-20%) to get Compound 148-3 (450 mg, 86%) as slightly yellow oil. ¹H NMR (300 MHz, CDCl₃): δ ppm 4.19-4.03 (m, 4H), 3.71-3.60 (m, 1H), 2.36-2.21 (m, 4H), 1.82-1.39 (m, 14H), 1.38-1.22 (m, 44H), 0.87 (t, J=7.0 Hz, 6H). MS (CI): m/z [M+H]⁺ 636.28.

### Synthesis of 1-(2-(bicyclo[2.2.2]octan-1-yl)ethyl) 17-(3-pentyloctyl) 9-((4-(dimethylamino) butanoyl)oxy)heptadecanedioate (C66)

A mixture of dimethylamino butanoic acid (164.05 mg, 0.98 mmol), EDC (536.13 mg, 2.79 mmol), and DMAP (93.96 mg, 0.77 mmol) in anhydrous dichloromethane (10 mL) was stirred at room temperature for 10 min. A solution of L48-3 (444 mg, 0.69 mmol) in anhydrous dichloromethane (5 mL) was added and the resultant mixture was stirred at room temperature for 4 h. The reaction mixture was concentrated under reduced pressure and the residue was purified by flash chromatography (the flash column was equilibrated with 10% triethylamine in hexane before use, eluent: ethyl acetate/hexane 0-50%) to give Compound C66 (450 mg, 86%). ¹H NMR (300 MHz, CDCl₃): δ ppm 4.86 (t, J=7.0 Hz, 1H), 4.07-4.0 (m, 4H), 2.32-2.20 (m, 14H), 1.86-1.73 (m, 1H), 1.63-1.44 (m, 18H), 1.43-1.19 (m, 41H), 0.87 (t, J=7.0 Hz, 6H); MS (CI): m/z [M+H]⁺ 749.20. Analytical HPLC column: Agela Durashell C18, 3 μm (Catalog No. DC930505-0), 4.6×150 mmol, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=10.7 min, purity: 87.94%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.1 min, purity: >99%.

### Synthesis of Compound C67

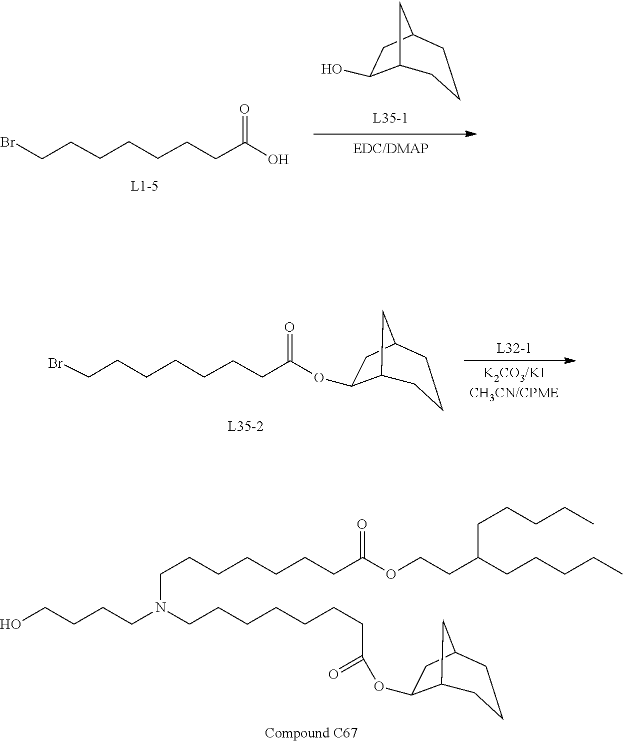 [SMILES: OC1CC2CCCC1C2 ; O=C(O)CCCCCCCBr ; O=C(CCCCCCCBr)OC1CC2CCCC1C2 ; CCCCCC(CCCCC)CCOC(=O)CCCCCCCN(CCCCO)CCCCCCCC(=O)OC1CC2CCCC1C2]

### Synthesis of (1R,5S,6S)-bicyclo[3.2.1]octane-6-yl 8 Bromo octanoate (L35-2)

To a solution of L1-5 (2.28 g, 10.30 mmol, 1.3 eq) and L35-1 (1.0 g, 7.93 mmol, 1.0 eq) in DCM (15 ml) was added EDC (4.5 g, 23.79 mmol, 3.0 eq) and DMAP (0.484 g, 4.0 mmol, 0.5 eq). The reaction was stirred at room temperature for 3 h and reduced under vacuum to dryness. The crude was purified by column (10% Ethyl acetate/hexane) to give L35-2 as colorless oil (1.4 g, 53%). ¹HNMR (CDCl₃) δ 4.94-4.96 (dd, 1H), 3.36-3.51 (t, 2H), 2.21-2.26 (m, 3H), 1.95-2.15 (m, 2H), 1.45-1.60 (m, 4H), 1.28-1.36 (m, 7H); CIMS m/z [M+H]⁺ 331.

### Synthesis of (1R,5S,6S)-bicyclo [3.2.1]octan-6-yl 8-((4-hydroxybutyl) (8-oxo-8-((3-pentyloctyl) oxy) octyl) amino) octanoate (Compound C67)

To a solution of L32-1 (480 mg, 1.2 mmol, 1.0 eq) and L35-2 (500 mg, 1.5 mmol, 1.3 eq) in a 1:1 mixture of ACN (12 ml) and CPME (12 ml) was added K₂CO₃ (oven dried: 497 mg, 3.6 mmol, 3.0 eq) and KI (100 mg, 0.6 mmol, 0.5 eq). The reaction mixture was heated at 80° C. for 22 h. After cooling to room temperature, the reaction mixture was filtered through celite. The celite cake was washed with ACN (50 ml), The combined filtrates were concentrated and purified by column (1 to 100% EA in Hexane) to give Compound C67 as colorless oil (370 mg, 47%). ¹HNMR (CDCl₃) δ: 6.70 (bs, 1H), 4.9 (dd, 1H), 4.04-4.09 (t, 2H), 5.01-5.03 (bs, 2H), 2.38-2.43 (m, 5H), 2.23-2.27 (m, 5H), 1.95-2.15 (m, 5H), 1.57-1.64 (m, 16H), 1.30-1.40 (m, 41H) 0.85-0.87 (t, 6H); CIMS m/z [M+H]⁺664.1; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=7.78 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=8.42 min, purity: 97.5%.

### Synthesis of Compound C68

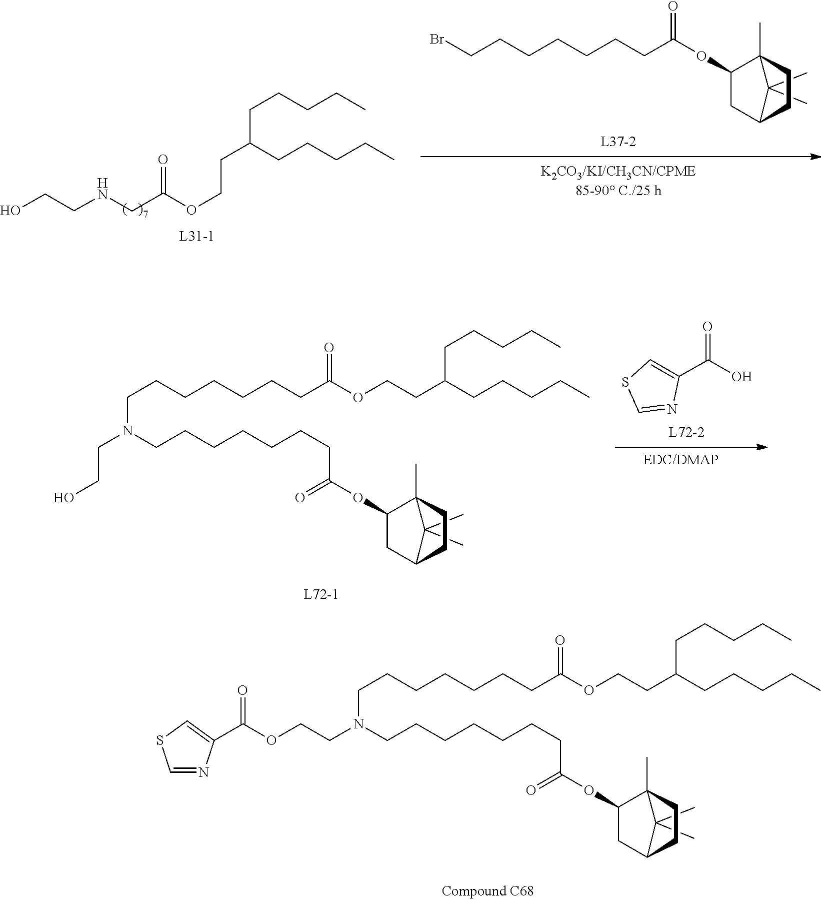

### Synthesis of 3-pentyloctyl 8-((2-hydroxyethyl)(8-oxo-8-(((1S,2R,4S)-1,7,7-trimethyl-bicyclo[2.2.1]heptan-2-yl)oxy)octyl)amino)octanoate (L72-1)

To a mixture of 3-pentyloctyl 8-((2-hydroxyethyl)amino)octanoate L31-1 (446 mg, 1.08 mmol) and L37-2 (446 mg, 1.08 mmol) in a mixture of AcCN/CPME (1:1, 10 mL) under nitrogen was added K₂CO₃ (447 mg, 3.24 mmol) followed by KI (90 mg, 0.54 mmol). The reaction mixture was heated at 80° C. for overnight under nitrogen atmosphere. After cooling to room temperature, the reaction mixture was filtered through celite, celite cake was washed with acetonitrile. The filtrate was concentrated to give crude product, which was purified by flash chromatography (SiO₂: 0 to 10% MeOH in DCM (1% NH₄OH) gradient) to yield L72-1 as colorless oil (505 mg, 68%). ¹H-NMR (300 MHz, CDCl₃) δ 4.89-4.85 (m, 1H), 4.07 (t, J=7.0 Hz, 2H), 3.51 (t, J=5.3 Hz, 2H), 2.62-2.21 (m, 10H), 1.99-1.15 (m, 44H), 1.03-0.86 (m, 18H); CIMS m/z [M+H]⁺ 664.3.

### Synthesis of heptadecan-9-yl 2-(1-(4-(benzyloxy)butyl)-4-(2-oxo-2-((3-pentyloctyl)oxy)ethyl)piperidin-4-yl)acetate (C₆₈)

To a solution of L72-1 (320 mg, 0.58 mmol) in DCM (10 mL) was added thiazole-4-carboxylic acid L72-2 (225 mg, 0.88 mmol) followed by DMAP (38 mg, 0.3 mmol) and EDC (225 mg, 1.2 mmol). The resulting mixture was stirred at room temperature under nitrogen atmosphere for 18 h. The reaction mixture was diluted with DCM (15 mL) and washed with brine (10 mL). The organic phase was dried over anhydrous Na₂SO₄.

Filtration and concentration provided crude material which was purified by flash column chromatography (SiO₂: ethyl acetate/hexane 0-100%) to yield Compound C68 as colorless oil (305 mg, 66%). ¹H-NMR (300 MHz, CDCl₃) δ 8.85 (d, J=2.2 Hz, 1H), 8.24 (s, 1H), 4.89-4.85 (m, 1H), 4.09 (t, J=6.6 Hz, 2H), 4.07 (t, J=7.1 Hz, 2H), 2.84 (t, J=5.8 Hz, 2H), 2.48 (t, J=6.3 Hz, 4H), 2.39-2.21 (m, 4H), 1.99-1.15 (m, 43H), 1.03-0.86 (m, 18H); CIMS m/z [M+H]⁺ 775.1; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=6.7 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=10.7 min, purity: >99%.

### Synthesis of Compound C69

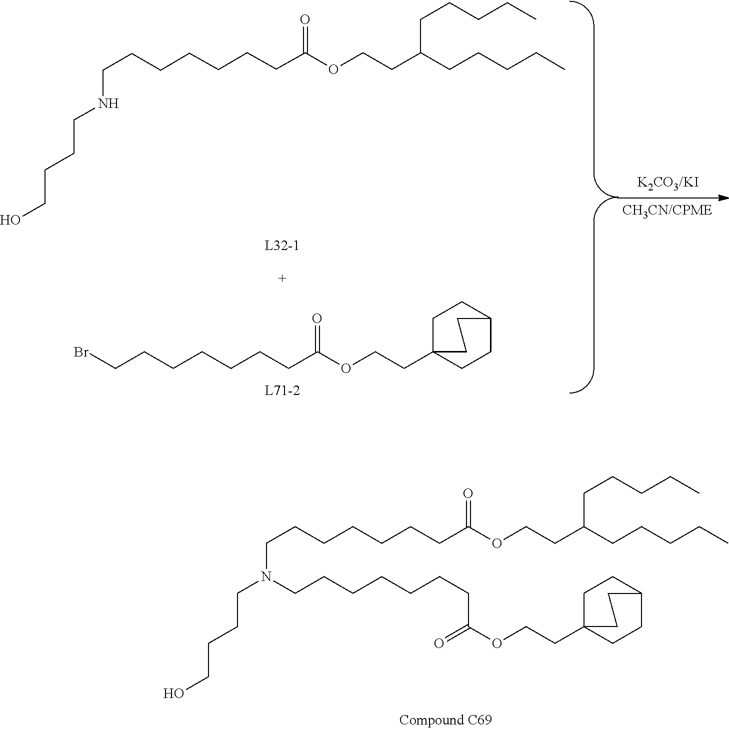 [SMILES: CCCCCC(CCCCC)CCOC(=O)CCCCCCCNCCCCO ; O=C(CCCCCCCBr)OCCC12CCC(CC1)CC2 ; CCCCCC(CCCCC)CCOC(=O)CCCCCCCN(CCCCO)CCCCCCCC(=O)OCCC12CCC(CC1)CC2]

### Synthesis of 2-(bicyclo[2.2.2]octan-1-yl)ethyl 8-((4-hydroxybutyl)(8-oxo-8-((3-pentyloctyl)oxy)octyl)amino)octanoate (Compound 69)

The starting material L32-1 (383 mg, 1 eq), K₂CO₃ (512 mg, 4 eq), and KI (154 mg, 1 eq) were mixed with CH₃CN/CPME (2/2 mL). After adding L71-2 (400 mg, 1.2 eq) to the above solution, the reaction mixture was stirred for 68 hours at 95° C. The reaction mixture was then filtered through filter paper The organic fraction was reduced under vacuum to obtain crude product which was then subjected to silica gel column using 0-10% MeOH in DCM with 1% NH₃·H₂O as eluent to afford Compound C69 as light-yellow oil (403 mg, 63%). ¹H-NMR (300 MHz, CDCl₃) δ 4.05 (tt, 4H), 3.61 (m, 2H), 2.67 (bs, 6H), 2.26 (tt, 4H), 1.85-1.42 (m, 26H), 1.41-1.10 (m, 32H), 0.87 (t, 6H). CIMS m/z [M+H]⁺ 692.1. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=5.72 min, purity: 99.8%; UPLC column: Thermo Scientific Hypersil GOLD C18, mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=10.82 min, purity: 95.0%.

### Synthesis of Compound C70

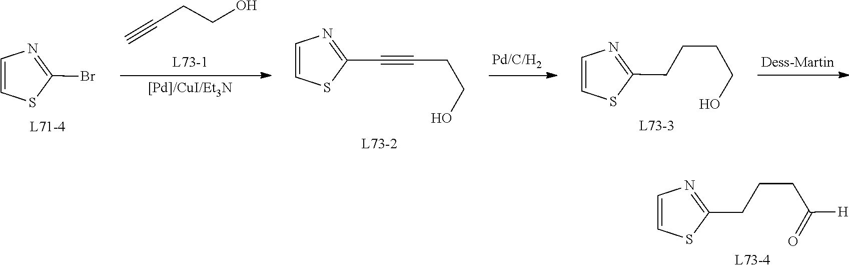

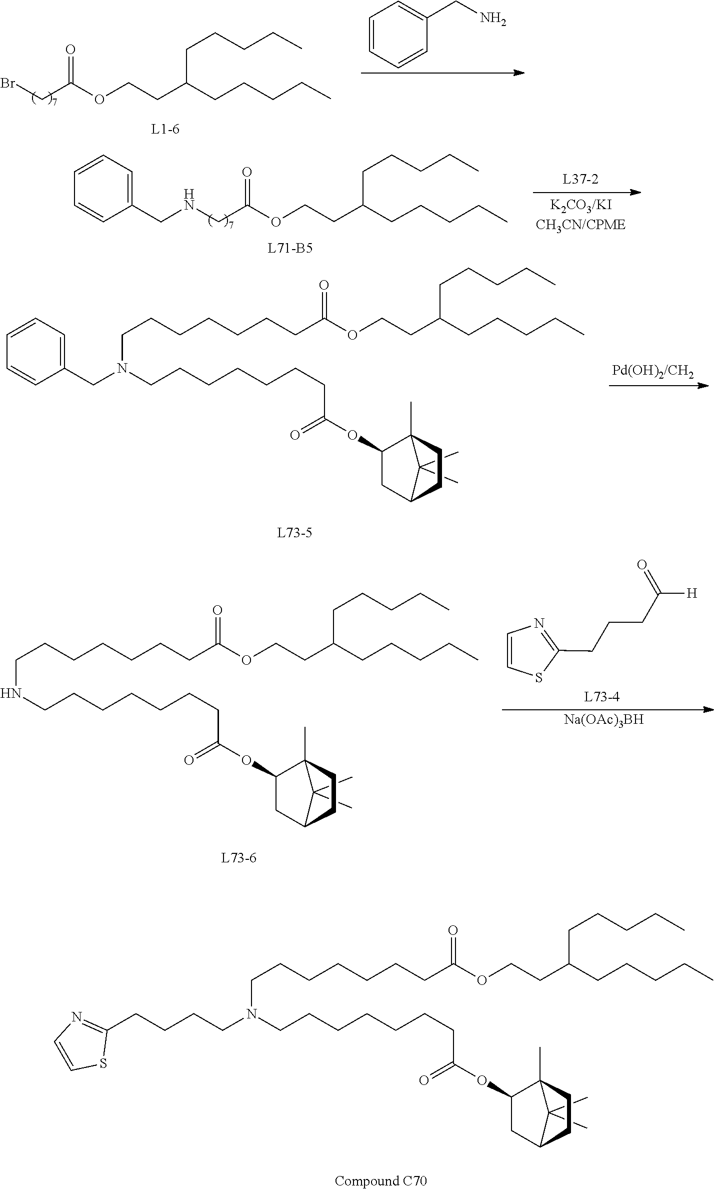

### Synthesis of 4-(thiazol-2-yl)but-3-yn-1-ol (L73-2)

2-Bromothiazole (2 g, 12.18 mmol) was dissolved in triethylamine (50 mL) under nitrogen atmosphere. CuI (70 mg, 0.36 mmol), Pd(PPh₃)₄ (281.5 mg, 0.24 mmol) and but-3-yn-1-ol (1.28 g, 18.2 mmol) were added and the solution was heated to reflux for 18 h. The solvent was removed under reduced pressure and the residue was purified by flash column chromatography (SiO₂: Hexane/EtOAc′ 0-100%) to get Compound L73-2 (1.44 g, 77%) as orange oil. ¹H-NMR (300 MHz, CDCl₃) δ 7.76 (d, 1H), 7.28 (d, 1H), 3.87 (t, 2H), 2.76 (t, 2H); APCI-MS: m/z [M+H]⁺ 154.

### Synthesis of 4-(thiazol-2-yl)butan-1-ol (L73-3)

To a solution of L73-2 (1.44 g, 9.35 mmol) in methanol (10 ml) was added 10% Pd/C (100 mg) at room temperature and the mixture was subjected to parr-shaker hydrogenator under hydrogen atmosphere (40 psi) for 18 h. Upon completion, the mixture was filtered through a celite pad and concentrated under reduced pressure and the crude was purified by column chromatography (SiO₂: DCM/DCM:MeOH (9:1)) to get Compound L73-3 (1.22 g, 76%) as orange oil. ¹H-NMR (300 MHz, CDCl₃) δ 7.66 (d, 1H), 7.19 (d, 1H), 3.68-3.64 (m, 2H), 3.09-3.04 (m, 2H), 1.90-1.81 (m, 2H), 1.69-1.66 (m, 2H); APCI-MS: m/z [M+H]⁺ 158.

### Synthesis of 4-(thiazol-2-yl)butanal (L73-4)

To an ice bath cooled solution of L73-3 (0.61 g, 3.88 mmol) in anhydrous DCM (15 mL) under nitrogen were added Dess-Martin periodinane (1.97 g, 4.65 mmol) in portion. The reaction mixture was warmed to room temperature and stirred for 2 h. The reaction was quenched by slow addition of saturated Na₂SO₃ (2 mL) and NaHCO₃ (2 mL) and stirred for a few minutes then acetic acid (1 ml) were added. The reaction mixture were concentrated under reduced pressure to dryness and the crude was purified by column chromatography (SiO₂: DCM/DCM:MeOH (9:1)) to get Compound L73-4 (0.3 g, 50%) as light yellow oil. ¹H-NMR (300 MHz, CDCl₃) δ 9.76 (s, 1H), 7.66 (d, 1H), 7.19 (d, 1H), 3.06 (t, 2H), 2.56 (t, 2H), 2.15 (t, 2H); APCI-MS: m/z [M+H]⁺ 156.

### Synthesis of 3-pentyloctyl 8-(benzylamino)octanoate (L71-5)

To 100 mL round bottom flask containing L1-6 (1.0 g, 2.46 mmol) and benzylamine (3.61 g, 33.68 mmol) was added ethanol (10 mL). The reaction mixture was subjected to vacuum/N₂ cycle (3×) and stirred under nitrogen at 70° C. for 24 h. The solvent was removed under vacuum to dryness and purified by flash chromatography (SiO₂: hexane/ethyl acetate 0-55%) to get Compound L71-5 (890 mg, 84%) as yellow oil. ¹H-NMR (300 MHz, CDCl₃) δ 7.32-7.25 (m, 5H), 4.09 (t, 2H), 3.77 (s, 2H), 2.63 (t, 2H), 2.27 (t, 2H), 1.57-1.24 (m, 29H), 0.87 (t, 6H); APCI-MS: m/z [M+H]⁺ 432.2.

### Synthesis of 3-pentyloctyl 8-(benzyl(8-oxo-8-(((1S,2S,4S)-1,7,7-trimethylbicyclo[2.2.1]heptan-2-yl)oxy)octyl)amino)octanoate (L73-5)

To a 100 mL round bottom flask containing L71-5 (0.88 g, 2.03 mmol), L37-2 (0.80 g, 2.24 mmol), K₂CO₃ (0.84 g, 6.09 mmol), KI (0.50 g, 3.04 mmol) was added anhydrous acetonitrile (5 mL) along with cyclopentylmethyl ether (CPME) (5 mL) and the reaction mixture was stirred under reflux at 90° C. for 48 h. After completion of the reaction the solvent was removed under vacuum and purified by flash chromatography (SiO₂: hexane (1% TEA)/ethyl acetate (0-100%)) to get Compound L73-5 (1.37 g, 95%) as colorless oil. ¹H-NMR (300 MHz, CDCl₃) δ 7.30-7.25 (m, 5H), 4.90 (dd, 1H), 4.09 (t, 2H), 3.51 (s, 2H), 2.36-2.26 (m, 8H), 2.0-1.85 (m, 1H), 1.85-1.10 (m, 46H), 0.89-0.81 (m, 15H); APCI-MS: m/z [M+H]⁺ 710.3.

### Synthesis of 3-pentyloctyl 8-((8-oxo-8-(((1S,2S,4S)-1,7,7-trimethylbicyclo[2.2.1]heptan-2-yl)oxy)octyl)amino)octanoate (L73-6)

To 100 mL round bottom flask containing L73-5 (1.37 g, 1.92 mmol) and 20% Pd(OH)₂/C (600 mg) was added methanol (10 mL). The reaction mixture was subjected to vacuum/N₂ cycle (3×) followed by another cycle of vacuum/H2 (3×). The hydrogen balloon was placed on the top of septum and left stirring for 4 days. Another 20% Pd(OH)₂/C (300 mg) was added and the reaction mixture was subjected to parr-shaker hydrogenator under hydrogen atmosphere (40 psi) for 18 h. The reaction mixture diluted with ethyl acetate (100 mL) and then filtered through Celite, washed with ethyl acetate and methanol. The solvent was removed under vacuum to dryness and used to the next step without further purification (SiO₂: hexane (10% triethyl amine)/ethyl acetate 0-38%) to get Compound L73-6 (1.15 g, 97%) as light-yellow oil. ¹H-NMR (300 MHz, CDCl₃) δ 4.90 (dd, 1H), 4.09 (t, 2H), 3.51 (s, 2H), 2.36-2.26 (m, 8H), 2.0-1.85 (m, 1H), 1.85-1.10 (m, 46H), 0.89-0.81 (m, 15H); APCI-MS: m/z [M+H]⁺ 620.3.

### Synthesis of 3-pentyloctyl 8-((8-oxo-8-(((1S,2S,4S)-1,7,7-trimethylbicyclo[2.2.1]heptan-2-yl)oxy)octyl)(4-(thiazol-2-yl)butyl)amino)octanoate (Compound C70)

To the mixture of L73-6 (599.16 mg, 0.96 mmol) and L73-4 (300 mg, 1.93 mmol) in 1,2-Dichloroethane (15 mL) was added Na(OAc)₃BH (610.38 mg, 2.88 mmol) and acetic acid (0.05 mL, 0.96 mmol). The reaction mixture was subjected to vacuum/N₂ cycle (3×) and stirred under nitrogen at room temperature for 18 h. The reaction was quenched by slow addition of saturated NaHCO₃ (100 mL) at 0° C. The aqueous phase was extracted using DCM (100 mL, 3×) and the combined organic phases were dried over anhydrous Na₂SO₄.

Filtration followed by concentration provided crude material which was loaded on 20 g flash silica column and was purified by flash chromatography (SiO₂: hexane/ethyl acetate (0 to 100%)) followed by another column using (DCM: MeOH: NH₄OH (9:1:0.1)) to yield Compound C70 (235 mg, 32%) as slightly yellow oil. ¹H-NMR (300 MHz, CDCl₃) δ 7.31-7.30 (m, 5H), 4.46 (s, 2H), 4.09-4.04 (m, 4H), 3.47-3.43 (m, 2H), 2.52-2.31 (m, 10H), 1.68-1.57 (m, 8H), 1.22 (t, 6H); APCI-MS: m/z [M+H]⁺ 420.3; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.6 min, purity: >99%; UPLC column: Waters Aquity UPLC® CSHTM, C18, 1.7 μm, 3.0×150 mm, (Part No. 186005302),mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.8 min, purity: >99%.

### Synthesis of Compound C71

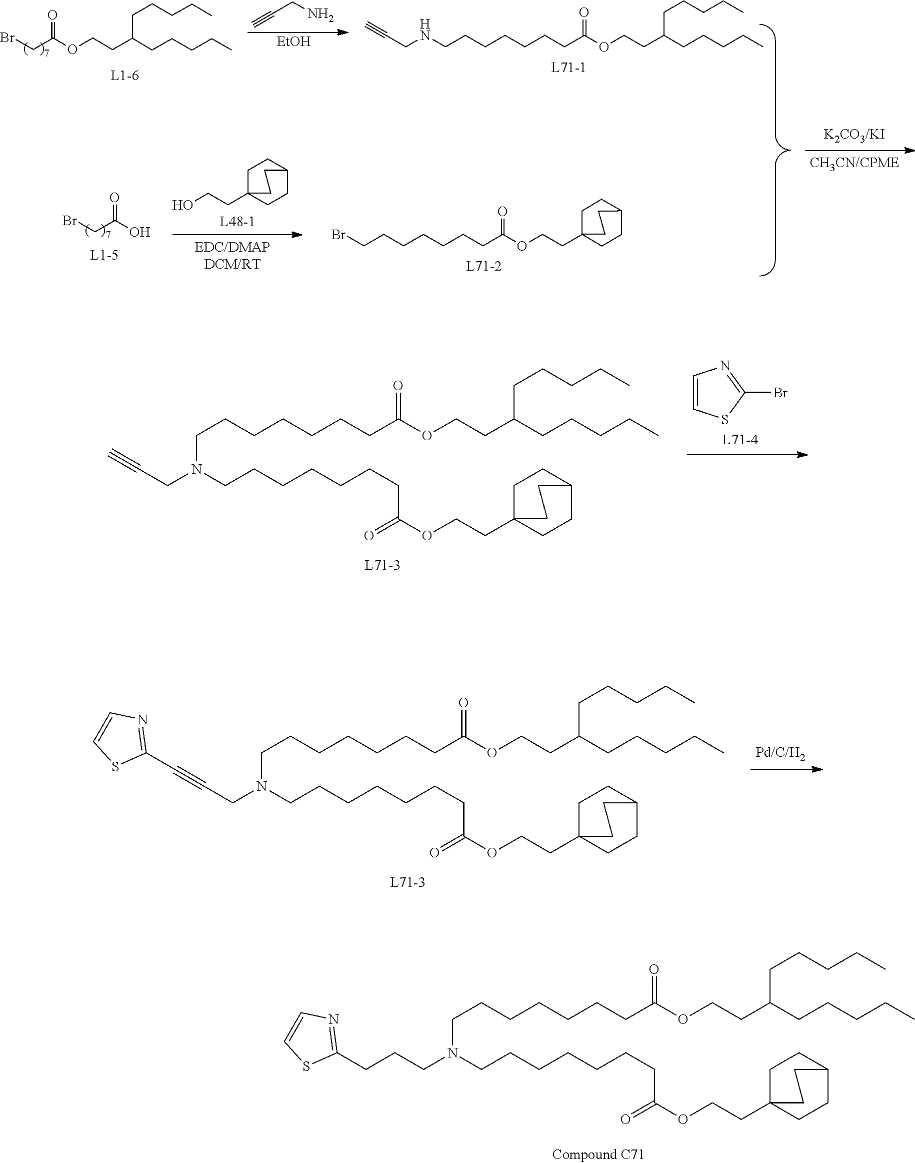

### Synthesis of 3-pentyloctyl 8-(prop-2-yn-1-ylamino)octanoate (L71-1)

A solution of L1-6 (1.0 g, 2.46 mM) and propargyl amine (1.36 g, 24.66 mM) in ethanol (40 mL) was refluxed for 18 h. After completion of the reaction, the solvent was removed under vacuum. Residual paste was purified by flash chromatography (SiO₂: ethyl acetate/hexane 0-100%) to get L71-1 (520 mg, 55%) as colorless oil. ¹H NMR (300 MHz, CDCl₃): δ ppm: 4.07 (t, J=7.0 Hz, 2H), 3.41 (d, J=2.0 Hz, 2H), 2.67 (t, J=7.0 Hz, 2H), 2.27 (t, J=7.0 Hz, 2H), 2.20 (t, J=2.0 Hz, 1H), 1.61-1.22 (m, 30H), 0.87 (t, J=7.5 Hz, 6H). MS (CI): m/z [M+H]⁺ 380.42.

### Synthesis of 2-(bicyclo[2.2.2]octan-1-yl)ethyl 8-bromooctanoate (L71-2)

To a 100 mL round bottom flask L1-5 (600 mg, 3.38 mM), EDC (1.49 g, 7.78 mM), were added in anhydrous dichloromethane (20 mL) and the reaction mixture was stirred for 15 min. To this was added DMAP (478.2 mg, 3.89 mM) and L48-1 (600 mg, 3.38 mM) in anhydrous dichloromethane (5 mL). The resulting mixture was stirred under nitrogen at room temperature for 16 h. The reaction mixture was concentrated under reduced pressure and the residue was purified by flash chromatography (SiO₂: ethyl acetate/hexane 0-20%) to get L71-2 (910 mg, 65%) as a clear oil. ¹H NMR (300 MHz, CDCl₃): δ ppm: 4.06 (t, J=7.0 Hz, 2H), 3.39 (d, J=2.0 Hz, 2H), 2.26 (t, J=7.0 Hz, 2H), 1.91-1.72 (m, 2H), 1.71-1.31 (m, 23H). MS (CI): m/z [M+H]⁺ 360.43.

### Synthesis of 2-(bicyclo[2.2.2]octan-1-yl)ethyl 8-((8-oxo-8-((3-pentyloctyl)oxy)octyl)(prop-2-yn-1-yl)amino)octanoate (L71-3)

A mixture of L71-1 (420 mg, 1.10 mM), L71-2 (477 mg, 1.33 mM), KI (275.5 mg, 1.65 mM) and potassium carbonate (458.7 mg, 3.32 mM) in anhydrous ACN (2.5 mL) and cyclopentylmethyl ether (CPME) (2.5 mL) was refluxed under nitrogen at 120° C. for 24 h. After cooling to room temperature, the reaction mixture was filtered through celite and the celite cake was washed with acetonitrile. The filtrate was concentrated to give crude product, which was purified by flash chromatography (the flash column was equilibrated with 10% triethylamine in hexane before use, eluent: ethyl acetate/hexane 0-50%) to get Compound L71-3 (410 mg, 56%). ¹H NMR (300 MHz, CDCl₃): δ ppm: 4.17-4.02 (m, 4H), 3.39 (d, J=2.0 Hz, 2H), 2.43 (t, J=7.0 Hz, 4H), 2.30 (t, J=7.0 Hz, 4H), 2.15 (s, 1H), 1.61-1.22 (m, 54H), 0.87 (t, J=7.5 Hz, 6H). MS (CI): m/z [M+H]⁺ 658.08.

### Synthesis of 2-(bicyclo[2.2.2]octan-1-yl)ethyl 8-((8-oxo-8-((3-pentyloctyl)oxy)octyl)(3-(thiazol-2-yl)prop-2-yn-1-yl)amino)octanoate (L71-5)

A mixture of L71-3 (235 mg, 0.357 mM), L71-4 (87.8 mg, 0.535 mM), Pd(PPh₃)₄ (41.2 mg, 0.03 mM) and copper iodide (13.6 mg, 0.07 mM) in triethylamine (5 mL) was refluxed under nitrogen for 18 h. Solvent was removed under vacuum and the residue was purified by flash chromatography (SiO₂: ethyl acetate/hexane 0-50%) to get L71-5 (146 mg, 55%). ¹H NMR (300 MHz, CDCl₃): δ ppm: 7.77 (d, J=7.0 Hz, 1H), 7.31 (d, J=7.0 Hz, 1H), 4.16-4.02 (m, 4H), 3.65 (s, 2H), 2.47 (t, J=8.0 Hz, 4H), 2.25 (t, J=8.0 Hz, 4H), 1.58-1.22 (m, 54H), 0.87 (t, J=7.5 Hz, 6H). MS (CI): m/z [M+H]⁺ 741.18.

### Synthesis of 2-(bicyclo[2.2.2]octan-1-yl)ethyl 8-((8-oxo-8-((3-pentyloctyl)oxy)octyl)(3-(thiazol-2-yl)propyl)amino)octanoate (Compound C71)

A mixture of L71-5 (182 mg, 0.245 mM), and Palladium (II) hydroxide (103.5 mg, 0.3 eq) in ethyl acetate (5 mL) was stirred under 1 atm hydrogen (balloon) at room temperature for 3 days. After completion of the reaction, the reaction flask was degassed, and the palladium was filtered using celite pad. The filtrate was concentrated under vacuum and the residue was purified by flash chromatography (SiO₂: ethyl acetate/hexane 0-50%) to get Compound C71 (40 mg, 22%). ¹H NMR (300 MHz, CDCl₃): δ ppm: 7.78 (d, J=7.0 Hz, 1H), 7.25 (d, J=7.0 Hz, 1H), 4.16-4.03 (m, 4H), 3.15-3.01 (m, 2H), 2.42-2.25 (m, 10H), 2.05-1.90 (m, 2H), 1.61-1.24 (m, 54H), 0.87 (t, J=7.5 Hz, 6H). MS (CI): m/z [M+H]⁺ 745.28. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: mL/min, column temperature: 20±2° C., detector: ELSD, t_{R}=8.2 min, purity: 95.95%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=11.56 min, purity: 87.14%.

### Synthesis of Compound C72

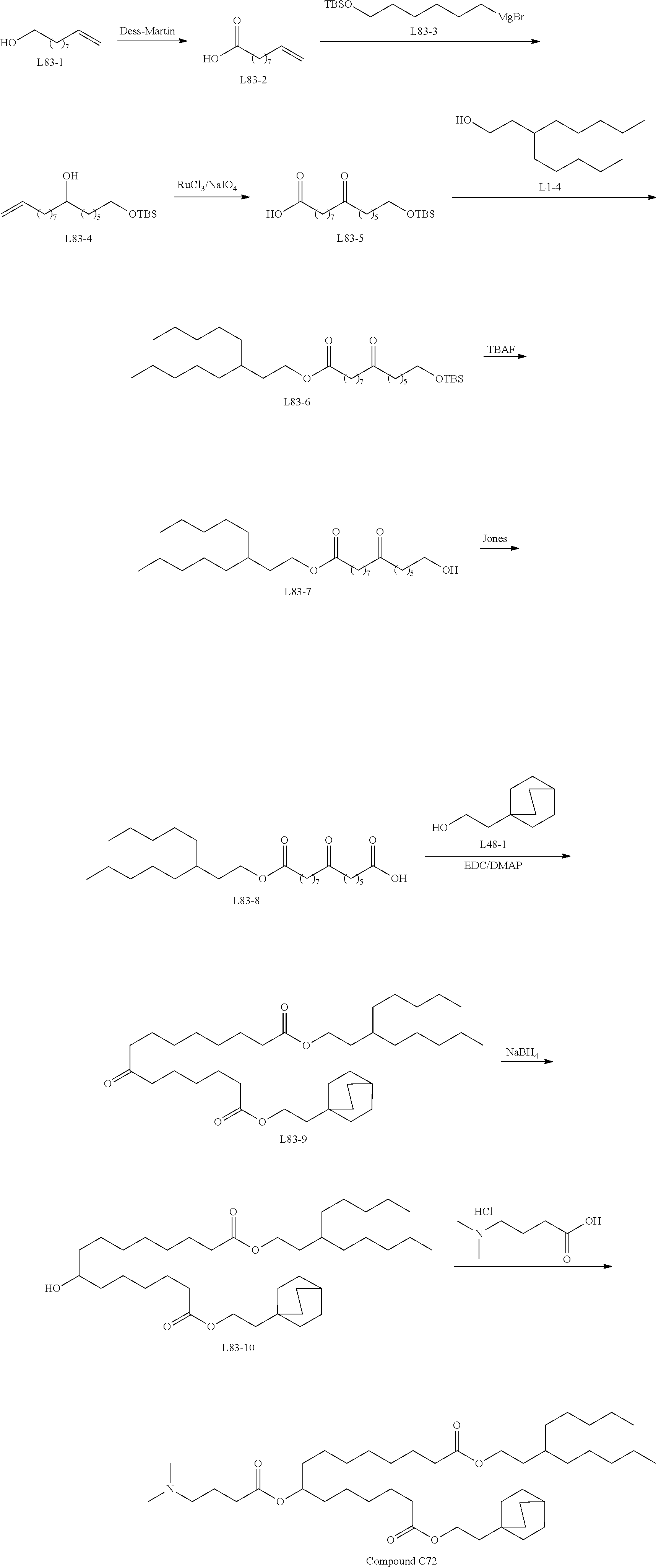

### Synthesis of dec-9-enal (L83-2)

Starting material L83-1 (5 g, 1 eq) was dissolved in DCM (130 mL) and cooled to 0° C. The Dess Martin periodinane (15.1 g, 1 eq) was added slowly. The reaction mixture was stirred at 0° C. for 10 minutes then brought to room temperature and stirred for 2 hours. To the reaction mixture was added 1 M NaOH (100 mL) and the organic layer was separated. Aqueous phase was extracted by DCM (100 mL×2). Combined organic phases were reduced under vacuum to give slightly cloudy crude product which was dissolved in a small amount of DCM and passed through a silica gel column using 0-5% EA in hexane as eluent to afford L83-2 as colorless liquid (2.8 g, 57%). ¹H-NMR (300 MHz, CDCl₃) δ 9.76 (s, 1H), 5.88-5.62 (m, 1H), 5.05-4.89 (m, 2H), 2.51-2.32 (m, 2H), 2.12-1.90 (m, 2H), 1.71-1.50 (2, 1H), 1.48-1.13 (m, 8H).

### Synthesis of 1-((tert-butyldimethylsilyl)oxy)hexadec-15-en-7-ol (L83-4)

Preparation of Grignard reagent: Magnesium turnings (329 mg, 4 eq) were put in a 50 mL round bottom flask and purged with nitrogen for 15 min. THF (3 mL), and 1,2-dibromoethane (58 μL, 0.2 eq) were added in sequence under nitrogen. The reaction was sealed and stirred for 3 min. ((6-bromohexyl)oxy)(tert-butyl)dimethylsilane (1.0 g, 1 eq) was dissolved in THF (10 mL) and dropped into the reaction mixture. The mixture was stirred at room temperature for 1 h and then at 60° C. for another hour. The dark grey solution was then cooled down to room temperature and used immediately. L83-2 (174 g, 1 eq) was added to the above Grignard reagent (~1.08 g in 13 mL THF, 3 eq) dropwise over 10 min. The reaction was stirred at room temperature for 2 hours, at which time, TLC (EA/hexane=1/4, R_{f}=0.7) showed completion of the reaction. Water (10 mL) was added slowly, and the mixture was extracted with ethyl acetate (EA) (10 mL×2). The organic layer was dried over Na₂SO₄ and reduced under vacuum to obtain crude product. The crude product was dissolved in a small amount of DCM and subjected to silica gel column using 0-25% EA in hexane as eluent to afford pure product L83-4 as colorless oil (300 mg, 72%). ¹H-NMR (300 MHz, CDCl₃) δ 5.88-5.62 (m, 1H), 5.05-4.89 (m, 2H), 3.67-3.50 (m, 3H), 2.11-1.94 (m, 2H), 1.72-1.13 (m, 27H), 0.90 (s, 9H), 0.04 (s, 6H). CIMS m/z [M+H]⁺ 371.1.

### Synthesis of 15-((tert-butyldimethylsilyl)oxy)-9-oxopentadecanoic acid (L83-5)

The starting material L83-4 (1.4 g, 1 eq), NaIO₄(6.4 g, 8 eq), and RuCl₃ (140 mg, 0.1 eq) were dissolved in ACN/CCl₄/H₂O (40/40/60 mL) respectively. The three components were combined and the resulting mixture was stirred at room temperature for 20 hours. When TLC (15% MeOH in DCM, R_{f}=0.4) showed completion of the reaction, H₂O (100 mL) was added to dilute the reaction and DCM (100 mL×2) was used to extract crude product. The organic solvent was reduced under vacuum to obtain crude product which was then subjected to silica gel column using 0-15% MeOH in DCM as eluent to afford L83-5 as light yellow oil (663 mg, 46%). ¹H-NMR (300 MHz, CDCl₃) δ 3.58 (t, 2H), 2.46-2.30 (m, 6H), 1.68-1.38 (m, 8H), 1.38-1.14 (m, 10H), 0.90 (s, 9H), 0.04 (s, 6H). CIMS m/z [M+H]⁺ 387.0.

### Synthesis of 3-pentyloctyl 15-((tert-butyldimethylsilyl)oxy)-9-oxopentadecanoate (L83-6)

To a solution of starting material L83-5 (600 mg, 1 eq) in DCM (18 mL) was added EDC/DMAP (1.19 g, 4 eq/209 mg, 1.1 eq). After stirring until the solution was clear, L1-4 (278 mg, 0.9 eq) was added in slowly. The reaction mixture was stirred at room temperature. When TLC (10% EA in Hexane, R_{f}=0.8) showed completion of the reaction, the solvent was reduced under vacuum to obtain crude product which was subjected to silica gel column using 0-10% EA in hexane as eluent to afford L83-6 as colorless oil (710 mg, 81%). ¹H-NMR (300 MHz, CDCl₃) δ 4.07 (t, 2H), 3.58 (t, 2H), 2.37 (t, 4H), 2.27 (t, 2H), 1.65-1.40 (m, 11H), 1.40-1.13 (m, 29H), 0.86-0.91 (m, 15H), 0.04 (s, 6H). CIMS m/z [M+H]⁺ 569.1.

### Synthesis of 3-pentyloctyl 15-hydroxy-9-oxopentadecanoate (L83-7)

The starting material L83-6 (650 mg, 1 eq) was added to TBAF (1 M in THF, 10 mL). The resulting mixture was stirred at room temperature for 20 hours. When TLC (40% EA in Hexane, R_{f}=0.6) showed completion of the reaction, the solvent was reduced under vacuum to obtain crude product which was subjected to silica gel column using 0-40% EA in Hexane as eluent to afford L83-7 as colorless oil in (462 mg, 89%). ¹H-NMR (300 MHz, CDCl₃) δ 4.07 (t, 2H), 3.63 (t, 2H), 2.47-2.32 (m, 4H), 2.27 (t, 2H), 1.65-1.40 (m, 11H), 1.40-1.13 (m, 29H), 0.88 (t, 6H). CIMS m/z [M+H]⁺ 455.1.

### Synthesis of 7,15-dioxo-15-((3-pentyloctyl)oxy)pentadecanoic acid (L83-8)

The starting material L83-7 (450 mg, 1 eq) was dissolved in THF (0.5 mL) and Jones reagent was added dropwise into the reaction. The Jones reagent was dropped into the reaction mixture until the resulting dark green color did not change any more. The reaction mixture was then stirred at room temperature for 30 minutes. When TLC (EA/hexane=1/1, R_{f}=0.7) showed completion of the reaction, the reaction was quenched with 2-propanol (0.5 mL), diluted with H₂O (5 mL), and extracted by DCM (5 mL×2). The organic fraction was reduced under vacuum to obtain crude product which was subjected to silica gel column using 0-50% EA in hexane as eluent to afford L83-8 as colorless oil (420 mg, 91%). ¹H-NMR (300 MHz, CDCl₃) δ 4.07 (t, 2H), 2.42-2.18 (m, 8H), 2.27 (t, 2H), 1.65-1.40 (m, 11H), 1.40-1.13 (m, 29H), 0.88 (t, 6H). CIMS m/z [M+H]⁺ 469.4.

### Synthesis of 1-(2-(bicyclo[2.2.2]octan-1-yl)ethyl) 15-(3-pentyloctyl) 7-oxopentadecanedioate (L83-9)

The starting material L83-8 (400 mg, 1 eq) and EDC/DMAP (655 mg, 4 eq/115 mg, 1.1 eq) were dissolved in DCM (15 mL). After stirring until the solution was clear, L48-1 (157 mg, 1.2 eq) was added to the above solution. The reaction mixture was monitored by TLC and stirred for 2 hours at room temperature. When TLC (EA/hexane=1/4, R_{f}=0.3) showed completion of the reaction, the volatile components were reduced under vacuum to obtain crude product which was subjected to silica gel column using 0-20% EA in Hexane as eluent to afford L83-9 as light-yellow oil (440 mg, 86%). ¹H-NMR (300 MHz, CDCl₃) δ 4.16-3.98 (m, 4H), 2.48-2.33 (m, 4H), 2.31-2.21 (m, 4H), 1.69-1.47 (m, 16H), 1.47-1.06 (m, 34H), 0.88 (t, 6H). CIMS m/z [M+H]⁺ 605.6.

### Synthesis of 1-(2-(bicyclo[2.2.2]octan-1-yl)ethyl) 15-(3-pentyloctyl) 7-hydroxypentadecanedioate (L83-10)

A solution of starting material L83-9 (440 mg, 1 eq) in THF/MeOH (8 mL/2 mL) was cooled to 0° C. NaBH₄ (30 mg, 1.1 eq) was then added. The resulting mixture was brought to room temperature and stirred for 3 hours. When TLC (EA/Hexane=1/4, R_{f}=0.6) showed completion of the reaction, the solvent was quenched by a few drops of H₂O, and reduced under vacuum to obtain crude product which was then subjected to silica gel column chromatography using 0-20% EA in Hexane as eluent to afford L83-10 as light-yellow oil (360 mg, 82%). ¹H-NMR (300 MHz, CDCl₃) δ 4.16-3.98 (m, 4H), 3.54 (bs, 1H), 2.31-2.21 (m, 4H), 1.69-1.47 (m, 14H), 1.47-1.06 (m, 40H), 0.88 (t, 6H). CIMS m/z [M+H]⁺ 607.5.

### Synthesis of 1-(2-(bicyclo[2.2.2]octan-1-yl)ethyl) 15-(3-pentyloctyl) 7-((4-(dimethylamino)butanoyl)oxy)pentadecanedioate (Compound C72)

Hydrochloric 4-(dimethylamino) butanoic acid (66 mg, 1.2 eq), DIPEA (0.1 mL) and DMAP (57 mg, 1.1 eq) were added to DCM (10 mL). The mixture was stirred until the solution was clear, then EDC (253 mg, 4 eq) was added. The reaction was stirred at room temperature for 10 minutes, and the starting material L83-10 (200 mg, 1 eq), dissolved in DCM, was added to the above solution. The reaction mixture was stirred for 5 hours at room temperature and concentrated under vacuum. The obtained crude product was then subjected to silica gel column using 0-10% MeOH in DCM with 1% NH₃·H₂O as eluent to afford Compound C72 as light-yellow oil (210 mg, 88%). ¹H-NMR (300 MHz, CDCl₃) δ 4.85 (quint, 1H), 4.05 (q, 4H), 2.38-2.19 (m, 12H), 1.81 (quint, 2H), 1.65-1.44 (m, 16H), 1.42-1.10 (m, 38H), 0.88 (t, 6H). CIMS m/z [M+H]⁺ 720.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.10 min, purity: >99%; UPLC column: Thermo Scientific Hypersil GOLD C18, mobile phase A: acetonitrile with 0.1% trifluoroacetic acid, mobile phase B: water with 0.1% trifluoroacetic acid, use gradient: A in B 40% to 100% in 5 min, then 100% for 15 min. Flow rate: mL/min, column temperature: 20±2° C., detector: CAD, t_{R}=12.67 min, purity: >99%.

### Synthesis of Compound C73

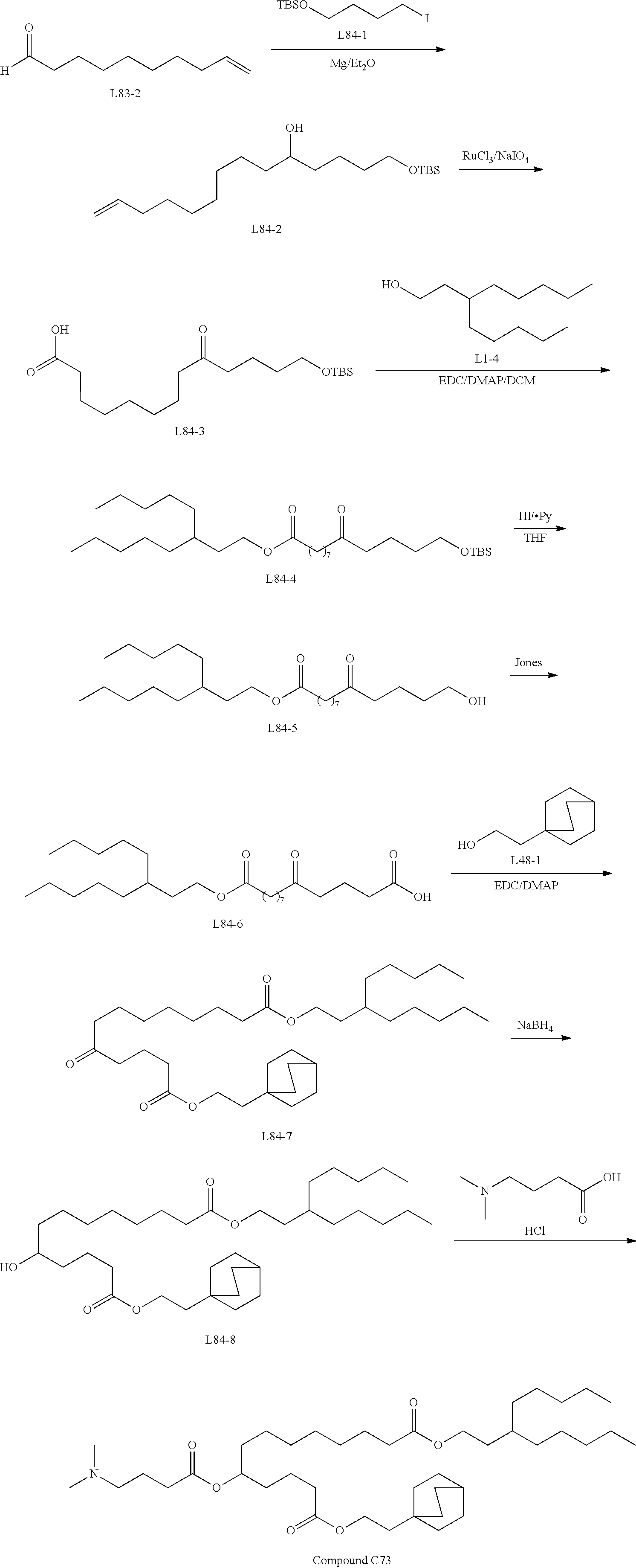

### Synthesis of 1-((tert-butyldimethylsilyl)oxy)tetradec-13-en-5-ol (L84-2)

Preparation of Grignard reagent: Magnesium turnings (1.4 g, 58.44 mmol) was put in a 250 mL round bottom flask and purged with nitrogen for 15 min. THF (3 mL), I₂ (20 mg) and 1,2-dibromoethane (100 μL) were added in sequence under nitrogen. The reaction was sealed and stirred for 3 min. L84-1 (9.16 g, 29.22 mmol) in ether solution was dropped into the reaction mixture. The mixture was stirred at room temperature for 1 h and refluxed for another hour. The dark grey solution was first cooled down to room temperature and then in ice-water bath. L83-2 (1.5 g, 9.74 mmol) in ether was added dropwise and the resulting mixture was stirred at room temperature overnight under nitrogen. Reaction mixture was cooled in ice-water bath again and quenched with sat. NH₄Cl solution (50 mL). Reaction mixture was extracted twice with ethyl acetate (150 mL, 100 mL). Organic layer was dried over Na₂SO₄ and concentrated under reduced pressure to obtain crude product, which was purified by flash chromatography (SiO₂: 0-10% EtOAc in hexane gradient) to yield L84-2 as colorless oil (2.2 g, 66% yield). ¹H-NMR (300 MHz, CDCl₃) δ 5.79 (m, 1H), 4.94 (m, 2H), 3.61 (m, 3H), 2.02 (m, 4H), 1.28-2.57 (m, 20H), 0.88 (s, 9H), 0.03 (s, 6H). CIMS m/z [M+H]⁺ 343.0, 325.1 (—H₂O).

### Synthesis of 13-((tert-butyldimethylsilyl)oxy)-9-oxotridecanoic acid (L84-3)

To a solution of A84-2 (2.0 g, 5.8 mmol) in a mixture of 1,2-dichloroethane (30 mL) and water (30 mL), RuCl₃ (38% in water, 163 mg, 5 mol %) and NaIO₄ (3.7 g, 17.4 mmol) were added to the reaction mixture successively and the resulting mixture was stirred at room temperature overnight. More of RuCl₃ (100 mg) NaIO₄ (2.47 g) were added and stirring continued for 24 hours. Ethyl acetate (150 mL) was added to the reaction mixture, filtered through celite and organic layer was washed with Na₂S203 solution (100 mL) and brine (100 mL). Organic layer was dried over Na₂SO₄ and concentrated under reduced pressure to obtain crude product which was purified by flash chromatography (SiO₂: 0-30% EtOAc in hexane gradient) to yield L84-3 as colorless oil (1.12 g, 54% yield). ¹H-NMR (300 MHz, CDCl₃) δ 3.59 (t, J=6.33 Hz, 2H), 2.34 (m, 6H), 1.6 (m, 8H), 1.28 (m, 6H), 0.86 (s, 9H), 0.03 (s, 6H), CIMS m/z [M+H]⁺ 359.0, 341.0 (—H₂O), [M−H]⁺ 357.0, 392.9 (+2H₂O).

### Synthesis of 3-pentyloctyl 13-((tert-butyldimethylsilyl)oxy)-9-oxotridecanoate (L84-4)

To a solution of L84-3 (1.0 g, 2.79 mmol) in DCM (20 mL) was added DMAP (170 mg, 1.39 mmol) and EDC (1.06 g, 5.58 mmol). Reaction mixture was stirred at room temperature for 15 min. L1-4 (668 mg, 1 3.34 mmol) in DCM was added and the reaction mixture was stirred at room temperature for 3h. Reaction mixture was diluted with DCM (50 mL), washed with water (25 mL) and brine (25 mL). DCM layer was dried over anhydrous Na₂SO₄. DCM layer was dried over anhydrous Na₂SO₄ and concentrated under reduced pressure to obtain crude residue which was purified by flash chromatography (SiO₂: 0-5% ethyl acetated in hexane gradient) to yield L84-4 as colorless oil (1.18 g, 78%). ¹H-NMR (300 MHz, CDCl₃) δ 4.06 (t, J=7.14 Hz, 2H), 3.59 (t, J=6.33 Hz, 2H), 2.38 (m, 4H), 2.27 (t, J=7.68 Hz, 2H), 1.52-1.59 (m, 10H), 1.24-1.29 (m, 23H), 0.87 (m, 15H), 0.03 (s, 6H), CIMS m/z [M+H]⁺ 541.1.

### Synthesis of 3-pentyloctyl 13-hydroxy-9-oxotridecanoate (L84-5)

To a solution of L84-4 (1.18 g, 2.18 mmol) in anhydrous THF (10.0 mL) in a teflon r.b. flask was added HF.Py (5.0 mL) at 0-5° C. (ice-water bath). Reaction mixture was warmed to room temperature and stirred for 1 h. The reaction mixture was diluted with DCM (100 mL) and washed with sat. NaHCO₃ solution until water layer pH shows basic (>7.0) with pH paper. Washed with Sat. brine (15 mL). Organic layer was dried over anhydrous Na₂SO₄ and concentrated under reduced pressure to obtain crude product. Crude product was purified by flash chromatography (SiO₂: 0-20% EtOAc in hexane gradient) to yield 800 mg (86% yield) of L84-5 as colorless oil. ¹H-NMR (300 MHz, CDCl₃) δ 4.06 (t, J=7.14 Hz, 2H), 3.61-3.65 (m, 2H), 2.42 (m, 4H), 2.27 (t, J=7.41 Hz, 2H), 1.52-1.59 (m, 11H), 1.24-1.29 (m, 23H), 0.87 (m, 6H).

### Synthesis of 5,13-dioxo-13-((3-pentyloctyl)oxy)tridecanoic acid (L84-6)

Jones reagent (Aldrich) was added dropwise to a stirred solution of L84-5 (0.8 g, 1.87 mmol) in acetone (10 mL) at ice-water bath, the addition is continued until characteristic orange color of the reagent persist for about 20 min. Reaction mixture was stirred at r.t. for 2 hr. The excess oxidant was destroyed with isopropanol (1.0 mL) and the solvents was removed under reduced pressure. The residue was partitioned between ethyl acetate (50 mL) and water (50 mL). Ethyl acetate was separated, and water layer was extracted with ethyl acetate (50 mL). the combined ethyl acetate layers were dried over anhydrous Na₂SO₄ and concentrated under reduced pressure to obtain crude product. Crude product was combined with another 70 mg crude compound and purified by flash chromatography (SiO₂: 0-50% EtOAc in hexane gradient) to yield 700 mg (85% yield) of L84-6 as colorless oil. ¹H-NMR (300 MHz, CDCl₃) δ 4.07 (t, J=7.14 Hz, 2H), 2.49 (t, J=7.41 Hz, 2H), 2.35-2.41 (m, 4H), 2.27 (t, J=7.41 Hz, 2H), 1.84-1.94 (m, 2H), 1.50-1.59 (m, 6H), 1.24-1.29 (m, 24H), 0.87 (m, 6H). CIMS m/z [M+H]⁺ 441.3.0, 323.3 (—H₂O).

### Synthesis of 1-(2-(bicyclo[2.2.2]octan-1-yl)ethyl) 13-(3-pentyloctyl) 5-oxotridecanedioate (L84-7)

To a solution of L84-6 (700 mg, 1.59 mmol) in DCM (20 mL) was added DMAP (193 mg, 1.59 mmol) and EDC (1.21 g, 6.36 mmol) at room temperature under nitrogen atm. Reaction mixture was stirred at room temperature for 10 min. L48-1 (292 mg, 1.9 mmol) in DCM (2.0 mL) was added and reaction mixture was stirred at room temperature for 3h. Reaction mixture was diluted with DCM (50 mL), washed with water (25 mL) and brine (25 mL). DCM layer was dried over anhydrous Na₂SO₄ and concentrated under reduced pressure to obtain crude residue, which was purified by flash chromatography (SiO₂: 0-10% ethyl acetated in hexane gradient) to yield L84-7 as colorless oil (750 mg, 82% yield). ¹H-NMR (300 MHz, CDCl₃) δ 4.05 (m, 4H), 2.42 (t, J=7.14 Hz, 2H), 2.34 (t, J=7.71 Hz, 2H), 2.24-2.29 (m, 4H), 1.84-1.91 (m, 2H), 1.50-1.57 (m, 12H), 1.24-1.37 (m, 32H), 0.87 (m, 6H). CIMS m/z [M+H]⁺ 577.5.

### Synthesis of 1-(2-(bicyclo[2.2.2]octan-1-yl)ethyl) 13-(3-pentyloctyl) 5-hydroxytridecanedioate (L84-8)

To a solution of L84-7 (750 mg, 1.3 mmol) in a mixture of THF:MeOH (10 mL, 8:1) at 0° C. was added NaBH₄ (98 mg, 2.6 mmol). Reaction mixture was stirred at room temperature for 2 h and quenched with water (2.0 mL). Solvent was reduced under vacuum, ethyl acetate (50 mL) was added to reaction mixture and washed with water (25 mL), brine (25 mL), dried over anhydrous Na₂SO₄ and concentrated under reduced pressure to obtain crude product. Crude product was purified by flash chromatography (SiO₂: 0-20% EtOAc in hexane gradient) to yield L84-8 as colorless oil (600 mg, 79% yield). ¹H-NMR (300 MHz, CDCl₃) δ 4.07 (m, 4H), 3.59 (m, 1H), 2.25-2.3 (m, 4H), 1.46-1.55 (m, 18H), 2.24-2.39 (m, 52H), 0.87 (m, 6H). CIMS m/z [M+H]⁺ 579.5.

### Synthesis of 1-(2-(bicyclo[2.2.2]octan-1-yl)ethyl) 13-(3-pentyloctyl) 5-((4-(dimethylamino)butanoyl) oxy)tridecanedioate (Compound 73)

To a solution of N, N-dimethyl butyric acid (207 mg, 1.24 mmol) in DCM (15 mL) was added DMAP (126 mg, 1.03 mmol) and EDC (793 mg, 4.14 mmol) at room temperature under nitrogen. Reaction mixture was stirred at room temperature for 10 min. L84-8 (600 mg, 1.03 mmol) in DCM (5.0 mL) was added to the reaction mixture and the reaction mixture was stirred at room temperature for 3h. Reaction mixture was diluted with DCM (75 mL), washed with water (50 mL) and brine (50 mL). DCM layer was dried over anhydrous Na₂SO₄ and concentrated under reduced pressure to yield crude product. Crude product was purified by flash chromatography (SiO₂: 0-20% EtOAc in Hexane (5% Et₃N) gradient) to yield C₇₃ as colorless oil (408 mg, 57%). ¹H-NMR (300 MHz, CDCl₃) δ 4.86 (m, 1H), 4.04 (m, 4H), 2.24-2.29 (m, 8H), 2.2 (s, 6H), 1.75 (m, 2H), 1.5-1.59 (m, 18H), 1.24-1.36 (m, 32H), 0.87 (m, 6H). CIMS m/z [M+H]⁺ 692.5. Analytical HPLC column: Agela Durashell C18, 4.6×50 mm, 3 μm (Catalog No. DC930505-0), mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=7.37 min, purity: >99.9%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=12.1 min, purity: >99.9%.

### Synthesis of Compound C74

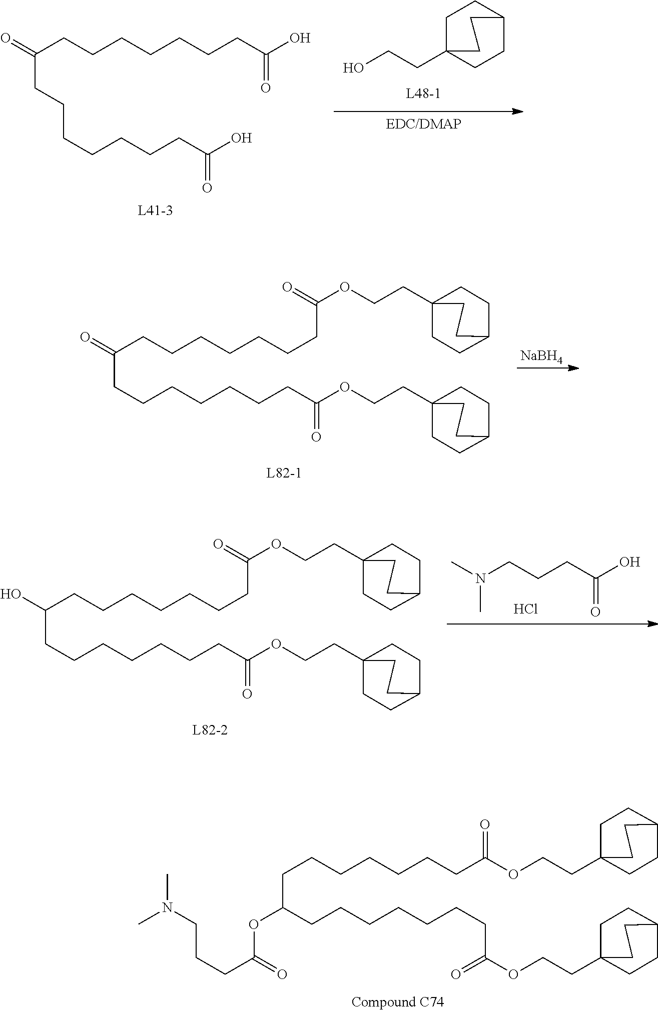

### Synthesis of bis(2-(bicyclo[2.2.2]octan-1-yl)ethyl) 9-oxoheptadecanedioate (L82-1)

A mixture of compound L41-3 (0.65 g, 2.0 mmol), DMAP (253 mg, 2.0 mmol), EDC (1.58 g, 8.2 mmol) and 2-(bicyclo[2.2.2]octan-1-yl)ethan-1-ol (L48-1) (0.44 g, 3.1 mmol) in dichloromethane (20 mL) was stirred at room temperature for 48 h and then reduced under vacuum. The residue was dissolved in dichloromethane (300 mL) and washed with saturated NaHCO₃, water and brine (80 mL×3). The combined organic layers were dried over anhydrous Na₂SO₄, and the solvent was reduced under vacuum. The crude was purified by column chromatography (40 g SiO₂: hexane/ethyl acetate 0-25%) to obtain compound L82-1 (0.71 g, 58%). ¹H-NMR (300 MHz, CDCl₃) δ 4.06-4.01 (m, 4H), 2.36 (t, 4H), 2.24 (t, 4H), 1.65-1.52 (m, 20H), 1.43-1.18 (m, 30H); CIMS m/z [M+H]⁺ 586.6.

### Synthesis of bis(2-(bicyclo[2.2.2]octan-1-yl)ethyl) 9-hydroxyheptadecanedioate (L82-2)

To a solution of compound L82-1 (0.23 g, 0.4 mmol) in methanol cooled to 5° C. in an ice bath was added sodium borohydride (16 mg, 0.43 mmol). After addition, cooling bath was removed, and the reaction mixture was stirred at room temperature for 2 h. The reaction mixture was adjusted with 1N aqueous HCl to pH 5 and the reaction mixture was concentrated in rotary evaporator. The residue was dissolved in DCM and washed with water. Organic layer dried over anhydrous Na₂SO₄, the solvent was reduced under vacuum, and the crude was purified by column chromatography (40 g SiO₂: 0 to 10% Methanol in DCM gradient) to obtain compound L82-2 as colorless oil (0.69 g, 93%). ¹H-NMR (300 MHz, CDCl₃) 4.05 (t, 4H), 3.56 (s, 1H), 2.25 (t, 4H), 1.64-1.29 (m, 55H); CIMS m/z [M+H]⁺ 588.6.

### Synthesis of bis(2-(bicyclo[2.2.2]octan-I-yl)ethyl) 9-((4-(dimethylamino)butanoyl)oxy) heptadecanedioate (Compound C74)

To a solution of compound 4-dimethylaminobutaric acid (0.404 g, 2.4 mmol) in dichloromethane (10 mL) were added DMAP (296 mg, 2.4 mmol) and EDCI (0.92 g, 4.8 mmol), followed by compound L82-2 (0.71 g, 1.2 mmol). The reaction mixture was stirred at room temperature for 4 h and reduced under vacuum. The residue was dissolved in dichloromethane (200 mL) and washed with brine (80 mL×3). The organic phase was dried over anhydrous Na₂SO₄, the solvent was reduced under vacuum, and the crude was purified by column chromatography (40 g SiO₂: 5% triethylamine in hexane/ethyl acetate 0-25%) to get final Compound C74 as colorless oil (0.65 g, 76%). ¹H-NMR (300 MHz, CDCl₃) δ 4.88-4.80 (m, 1H), 4.04 (t, 4H), 2.42-2.20 (m, 9H), 2.16 (s, 6H), 1.82-1.47 (m, 25H), 1.41-1.25 (m, 30H); CIMS m/z [M+H]⁺ 702.6. Analytical HPLC column: Agela Durashell C18, 3 m (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=7.38 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=11.2 min, purity: >94.3%.

### Synthesis of Compound C75

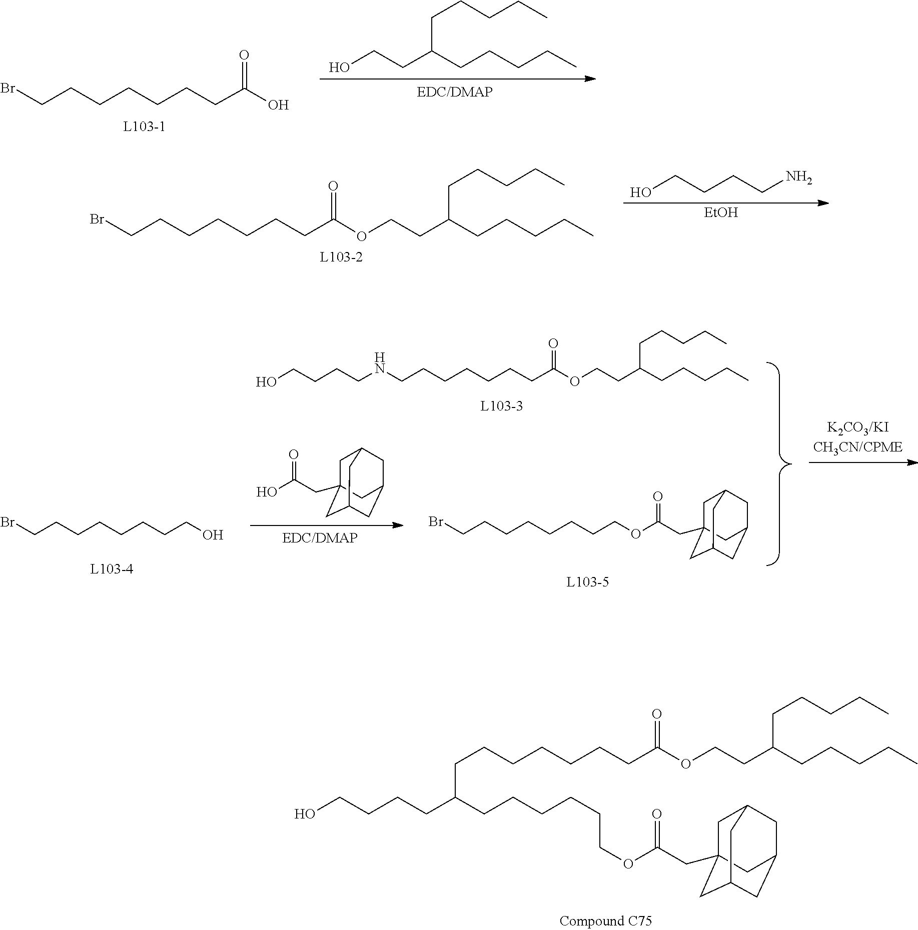

### Synthesis of 3-pentyloctyl 8-bromooctanoate (L103-2)

A solution of L103-1 (3.58 g, 16.04 mmol) in DCM (50 mL) EDC (7.08 g, 37.02 mmol) and DMAP (1.52 g, 12.34 mmol) was left to stir for 20 min before 3-pentyloctan-1-ol (2.48 g, 12.34 mmol) was added in a DCM solution (10 mL). After 3 h the reaction mixture was concentrated under reduced pressure and purified using column chromatography (SiO₂: 40 g, 0-2% ethyl acetate in hexane gradient) to afford L103-2 as clear colorless oil (4.01 g, 80%). ¹H-NMR (300 MHz, CDCl₃) δ 4.08 (t, J=7.2 Hz, 2H), 3.40 (t, J=6.7 Hz, 2H), 2.28 (t, J=7.4 Hz, 2H), 1.85 (t, J=7.3 Hz, 2H), 1.64-1.53 (m, 4H), 1.45-1.21 (m, 23H), 0.88 (t, J=6.9 Hz, 6H).

### Synthesis of 3-pentyloctyl 8-((4-hydroxybutyl)amino)octanoate (L103-3)

To a solution of L103-2 (3.00 g, 7.39 mmol) in ethanol (30 mL), was added 4-amino-1-butanol (6.58 g, 73.9 mmol) as a solution in ethanol (10 mL). The reaction mixture was heated to 70° C. and stirred for 20 h. The reaction mixture was concentrated under reduced pressure and the remaining residue was dissolved in DCM (100 mL). The organic layer was washed with water (50 mL×3), then brine (50 mL×3), then dried using anhydrous sodium sulfate, and concentrated under reduced pressure. The crude product was purified using column chromatography (SiO_{2:40} g, 0-10% methanol, 0-1% NH₄OH in dichloromethane gradient) to afford L103-3 as a clear colorless oil (1.75 g, 57%). ¹H-NMR (300 MHz, CDCl₃) δ 4.07 (t, J=7.2 Hz, 2H), 3.56 (t, J=4.8 Hz, 2H), 2.66-2.57 (m, 4H), 2.27 (t, J=7.4 Hz, 2H), 1.68-1.46 (m, 10H), 1.27 (d, J=16.8 Hz, 23H), 0.87 (t, J=6.7 Hz, 6H). CIMS m/z [M+H]⁺ 414.0.

### Synthesis of 8-bromooctyl 2-((3r,5r,7r)-adamantan-1-yl)acetate (L103-5)

To a solution of adamantane-1-acetic acid (1.76 g, 9.08 mmol) in DCM (50 mL), was added EDC (5.20 g, 27.24 mmol) and DMAP (1.11 g, 9.08 mmol). The reaction was stirred for 20 min before L103-4 (2.47 g, 11.80 mmol) was added as a DCM solution (10 mL). After 20 h, the reaction mixture was concentrated under reduced pressure and purified using column chromatography (SiO₂: 40 g, 0-2% ethyl acetate in hexane gradient) to afford L103-5 as a clear colorless oil (2.33 g, 65%). ¹H-NMR (300 MHz, CDCl₃) δ 4.04 (t, J=6.6 Hz, 2H), 3.41 (t, J=6.9 Hz, 2H), 2.06 (s, 2H), 1.97 (s, 3H), 1.84 (q, J=7.2 Hz, 2H), 1.73-1.57 (m, 14H), 1.45-1.31 (m, 8H).

### Synthesis of 3-pentyloctyl 8-((8-(2-((3r,5r,7r)-adamantan-1-yl)acetoxy)octyl)(4-hydroxybutyl)amino)octanoate (Compound C75)

To a solution of L103-3 (1.75 g, 4.24 mmol) and L103-5 (2.29 g, 5.94 mmol) in CPME and ACN (1:1, 30 mL) was added KI (0.70 g, 4.24 mmol) and K₂CO₃ (1.76 g, 12.72 mmol). The reaction mixture was heated to 90° C. and stirred for 90 h. The reaction mixture was concentrated under reduced pressure and the product was purified using column chromatography (SiO₂: 80 g, 0-10% methanol, 1% NH₄OH in dichloromethane gradient) to afford Compound C75 as colorless oil (1.95 g, 64%). ¹H-NMR (300 MHz, CDCl₃) δ 4.07-4.01 (m, 4H), 3.48-3.58 (2H), 2.41 (t, J=7.8 Hz, 6H), 2.27 (s, 2H), 2.06 (s, 2H), 1.96 (s, 3H), 1.68-1.55 (m, 28H), 1.31-1.25 (m, 29H), 0.88 (t, J=6.9 Hz, 6H). CIMS m/z [M+H]⁺ 719.0. Analytical HPLC column: Agela Durashell C18, 4.6×50 mm, 3 μm (Catalog No. DC930505-0), mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: mL/min, column temperature: 20±2° C., detector: ELSD, t_{R}=6.78 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=11.22 min, purity: >99%.

### Synthesis of Compound C79

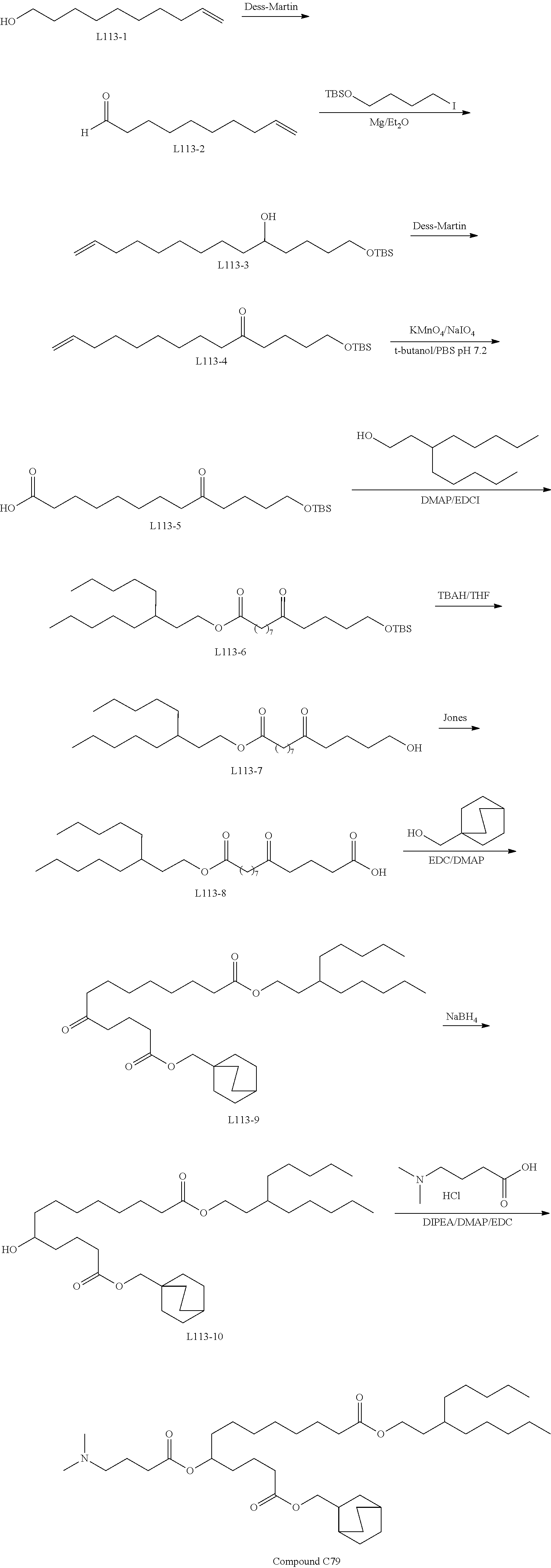

### Synthesis of dec-9-enal (L113-2)

To a stirring solution of L113-1 in DCM (200 mL) under nitrogen, was added Dess-Martin periodinane (39.08 g, 92.15 mmol), and the reaction mixture was stirred at 0° C. for 1 h and at RT for another 2 h. The reaction was quenched with a sat. aq. solution of sodium thiosulfate (100 mL) and sat. aq. solution of NaHCO₃until the pH was neutral (250 mL). The organic layer was separated, and the aqueous layer was further extracted with DCM (2×150 mL). The organic layers were combined and washed with water (150 mL×3) and brine (150 mL×3), then it was dried over anhydrous sodium sulfate. The organic layer was then concentrated under reduced pressure, and the residue was purified using column chromatography (SiO₂: 320g using a gradient of 0-10% EtOAC in hexanes). The fractions containing the product were combined and concentrated to afford L113-2 as colorless oil (9.02 g, 76%). ¹H-NMR (300 MHz, CDCl₃) δ 9.76 (t, J=1.8 Hz, 1H), 5.84-5.73 (m, 1H), 5.02-4.90 (m, 2H), 2.42 (td, J=7.3, 1.8 Hz, 2H), 2.03 (q, J=7.0 Hz, 2H), 1.61 (q, J=7.2 Hz, 2H), 1.37-1.31 (m, 8H).

### Synthesis of 1-((tert-butyldimethylsilyl)oxy)tetradec-13-en-5-ol (L113-3)

Magnesium turnings (4.07 g, 167.65 mmol) was put in a 250 mL round bottom flask and purged with nitrogen for 15 min. Diethyl ether (40 mL), iodine (20 mg) and 1,2-dibromoethane (100 μL) were added in sequence under nitrogen. The reaction was sealed and stirred for 15 min. Tert-butyl(4-iodobutoxy)dimethylsilane (26.34 g, 83.82 mmol) in ether solution (20 mL) was dropped into the reaction mixture. The mixture was stirred at room temperature for 1 h and refluxed for another hour. The dark grey solution was first cooled down to room temperature and then in an ice-water bath. L113-2 (4.31 g, 27.94 mmol) in ether was added dropwise and the resulting mixture was stirred at room temperature overnight under nitrogen. Reaction mixture was cooled in ice-water bath again and quenched with sat. aq. NH₄Cl solution (200 mL). Reaction mixture was extracted with ethyl acetate (200 mL×3). The organic layer was dried over anhydrous Na₂SO₄ and concentrated under reduced pressure to obtain crude product, which was purified by flash chromatography (SiO₂: 220 g, 0-10% EtOAc in hexane gradient) to yield L113-3 as colorless oil (5.5 g, 57%). ¹H-NMR (300 MHz, CDCl₃) δ 5.58-5.93 (IH), 4.76-5.10 (2H), 3.39-3.81 (3H), 1.90-2.14 (2H), 1.36 (dd, J=23.0, 18.3 Hz, 18H), 0.89 (t, J=2.8 Hz, 9H), 0.04 (s, 6H). CIMS m/z [M+H]⁺ 343, 325 (—H₂O).

### Synthesis of 1-((tert-butyldimethylsilyl)oxy)tetradec-13-en-5-one (L113-4)

To a solution of L113-3 (2.50 g, 7.30 mmol) in DCM (20 mL) in an ice bath, was added Dess-Martin reagent (1.20 g, 8.76 mmol). The reaction mixture was taken out of the ice bath and left to stir at room temperature for 4 h. The reaction mixture was quenched with a sat. aq. solution of sodium thiosulfate (30 mL) and sat. aq. solution of sodium bicarbonate (30 mL). The reaction mixture was then extracted with ethyl acetate (50 mL×3) and the organic layer was washed with water (50 mL×3), then brine (50 mL×3) then it was dried with anhydrous sodium sulfate and concentrated under reduced pressure leaving behind 4.01 g of L113-4 as a crude material which was used in the next step without any further purification. CIMS m/z [M+H]⁺ 341.3.

### Synthesis of 13-((tert-butyldimethylsilyl)oxy)-9-oxotridecanoic acid (L113-5)

In a 50 mL RBF, sodium periodate (6.23 g, 19.12 mmol) was added followed by phosphate buffer (pH 7.2, 30 mL) and the mixture was left to stir until a clear solution was observed. After that, KMnO₄ (1.15 g, 7.28 mmol) was added, and the mixture was left to stir for 15 min. In another 50 mL RBF containing the crude of L113-4 (4.01 g), tert-butanol (30 mL) was added followed by the permanganate solution and the reaction mixture was left to stir for 3 h. The reaction was quenched with saturated aqueous solution of sodium thiosulfate (100 mL) and the product was extracted using ethyl acetate (200 mL×3). The organic layer was washed with saturated aqueous solution of sodium sulfate (100 mL×1), saturated aqueous solution of ammonium chloride (100 mL×3), and brine (100 mL×3). The organic layer was dried with anhydrous sodium sulfate and concentrated under reduced pressure then purified with flash chromatography (SiO₂: 40 g, 0-10% methanol in dichloromethane gradient) to afford L113-5 as a colorless oil (2.44 g, 93%). ¹H-NMR (300 MHz, CDCl₃) δ 3.60 (s, 2H), 2.41-2.33 (m, 6H), 1.61-1.51 (m, 8H), 1.27 (d, J=16.0 Hz, 6H), 0.87 (s, 9H), 0.03 (s, 6H). CIMS m/z [M+H]⁺ 359.3.

### Synthesis of 3-pentyloctyl 13-((tert-butyldimethylsilyl)oxy)-9-oxotridecanoate (L113-6)

To a solution of L113-5 (2.44 g, 6.80 mmol) in DCM (20 mL), was added DMAP (831 mg, 6.80 mmol) and EDC (3.26 g, 17.01 mmol). The reaction mixture was stirred at room temperature for 15 min. 3-Pentyloctan-1-ol (1.36 g, 6.80 mmol) in DCM (5 mL) was added and the reaction mixture was stirred at room temperature overnight. The reaction mixture was concentrated under reduced pressure to obtain crude residue which was purified by flash chromatography (SiO₂: 40 g, 0-5% ethyl acetated in hexane gradient) to yield L113-6 as colorless oil (1.65 g, 45%). ¹H-NMR (300 MHz, CDCl₃) 4.07 (s, 2H), 3.60 (s, 2H), 2.39 (d, J=10.2 Hz, 4H), 2.27 (s, 2H), 1.57-1.55 (m, 10H), 1.27 (d, J=13.5 Hz, 23H), 0.90-0.85 (m, 15H), 0.04 (s, 6H). CIMS m/z [M+H]⁺ 541.5.

### Synthesis of 3-pentyloctyl 13-hydroxy-9-oxotridecanoate (L113-7)

To a 250 mL RBF containing a solution of L113-6 (1.65 g, 3.05 mmol) in THF (10 mL), was added a solution of TBAF in THF (1M, 10 mL). The reaction was left to stir at RT for 1 h. The reaction mixture was concentrated under reduced pressure and purified using column chromatography (SiO₂: 40 g, 0-50% ethyl acetate in hexane gradient) to yield L113-7 as a colorless oil (1.28 g, 98%). ¹H-NMR (300 MHz, CDCl₃) δ 4.07 (t, J=7.0 Hz, 2H), 3.63 (t, J=5.9 Hz, 2H), 2.42 (dt, J=16.4, 7.2 Hz, 4H), 2.27 (t, J=7.4 Hz, 2H), 1.69-1.53 (m, 10H), 1.27 (d, J=14.9 Hz, 23H), 0.91-0.85 (m, 6H); CIMS m/z [M+H]⁺ 409.4 (—H₂O).

### Synthesis of 5,13-dioxo-13-((3-pentyloctyl)oxy)tridecanoic acid (L113-8)

Jones reagent (chromium trioxide in diluted sulfuric acid) was added dropwise to a stirred solution of L113-7 (1.28 g, 3.00 mmol) in acetone (20 mL) at ice-water bath. The addition was continued until the characteristic orange color of the reagent persisted for about 20 min. Reaction mixture was stirred at room temperature for 2 h. The reaction was quenched with isopropanol (20 mL) and water (50 mL). The reaction mixture was concentrated under reduced pressure. The concentrated solution was further diluted with water (50 mL), then the product was extracted with dichloromethane (100 mL×4). The organic layer was dried over anhydrous Na₂SO₄ and concentrated under reduced pressure to obtain L113-8 as colorless oil (1.26 g, 95%). ¹H-NMR (300 MHz, CDCl₃) δ 7.26 (s, 2H), 4.07 (t, J=7.0 Hz, 2H), 2.49 (t, J=7.0 Hz, 2H), 2.38 (td, J=7.2, 2.1 Hz, 4H), 2.27 (t, J=7.4 Hz, 2H), 1.89 (t, J=7.2 Hz, 2H), 1.56 (q, J=6.8 Hz, 6H), 1.27 (d, J=14.3 Hz, 23H), 0.87 (t, J=6.9 Hz, 6H). CIMS m/z [M+H]⁺ 441.4.

### Synthesis of 1-(bicyclo[2.2.2]octan-1-ylmethyl) 13-(3-pentyloctyl) 5-oxotridecanedioate (L113-9)

To a solution of L113-8 (1.22 g, 2.77 mmol) in DCM (25 mL) was added DMAP (307 mg, 2.52 mmol) and EDC (1.93 g, 10.07 mmol) at room temperature under nitrogen atm. The reaction mixture was stirred at room temperature for 20 min. Bicyclo[2.2.2]octan-1-ylmethanol (353 mg, 2.52 mmol) in DCM (5.0 mL) was added and the reaction mixture was stirred at room temperature overnight. The reaction mixture was concentrated under reduced pressure to obtain crude residue, which was purified by flash chromatography (SiO₂: 40 g, 0-5% ethyl acetated in hexane gradient) to yield L113-9 as colorless oil (1.12, 79% yield). ¹H-NMR (300 MHz, CDCl₃) δ 4.07 (t, J=7.2 Hz, 2H), 3.67 (s, 2H), 2.48-2.24 (m, 8H), 1.88 (t, J=7.2 Hz, 2H), 1.59-1.53 (m, 14H), 1.42-1.24 (m, 28H), 0.87 (t, J=6.7 Hz, 6H).

### Synthesis of 1-(bicyclo[2.2.2]octan-1-ylmethyl) 13-(3-pentyloctyl) 5-hydroxytridecanedioate (L113-10)

To a solution of L113-9 (1.12 g, 1.99 mmol) in a mixture of THF and MeOH (50 mL, 4:1) at 0° C. was added NaBH₄(151 mg, 3.98 mmol). The reaction mixture was stirred at room temperature for 1 h and quenched with ice cold HCl (1N) until the solution reached pH 6. The THF was evaporated, and the product was extracted with dichloromethane (30 mL×3). The organic layer was then washed with water (30 mL×3), brine (30 mL×3), then dried over anhydrous Na₂SO₄ and concentrated under reduced pressure to obtain crude product. Crude product was purified by flash chromatography (SiO₂: 0-20% EtOAc in hexane gradient) to yield L113-10 as colorless oil (850 mg, 76% yield). ¹H-NMR (300 MHz, CDCl₃) δ 4.07 (s, 2H), 3.68 (s, 2H), 3.51-3.65 (1H), 2.34-2.28 (m, 4H), 1.57-1.53 (m, 14H), 1.46-1.25 (m, 34H), 0.88 (t, J=6.9 Hz, 6H). CIMS m/z [M+H]⁺ 565.5.

### Synthesis of 1-(bicyclo[2.2.2]octan-1-ylmethyl) 13-(3-pentyloctyl) 5-((4-(dimethylamino) butanoyl)oxy)tridecanedioate (Compound C79)

To a solution of 4-(dimethylamino)butanoic acid hydrochloride (207 mg, 1.24 mmol) in DCM (22 mL), was added DIPEA (1.0 mL, 5.74 mmol) DMAP (184 mg, 2.11 mmol), and EDC (1.15, 6.02 mmol) at room temperature under nitrogen. The reaction mixture was stirred at room temperature for 20 min. L113-10 (850 mg, 1.50 mmol) in DCM (8.0 mL) was added to the reaction mixture and the reaction mixture was stirred at room temperature overnight. The reaction mixture was concentrated under reduced pressure to yield crude product which was purified by flash chromatography (SiO₂: 0-20% EtOAc in Hexane (1% Et₃N) gradient) to yield Compound C79 as colorless oil (906 mg, 89%). ¹H-NMR (300 MHz, CDCl₃) δ 4.81-4.92 (1H), 4.07 (s, 2H), 3.67 (s, 2H), 2.28 (q, J=7.8 Hz, 8H), 2.21 (s, 6H), 1.78 (s, 2H), 1.59-1.53 (m, 19H), 1.39-1.25 (m, 29H), 0.88 (t, J=6.9 Hz, 6H). CIMS m/z [M+H]⁺ 678.6. Analytical HPLC column: Agela Durashell C18, 4.6×50 mm, 3 μm (Catalog No. DC930505-0), mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=7.33 min, purity: 99.5%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=10.85 min, purity: 95.8%.

### Synthesis of Compound C80

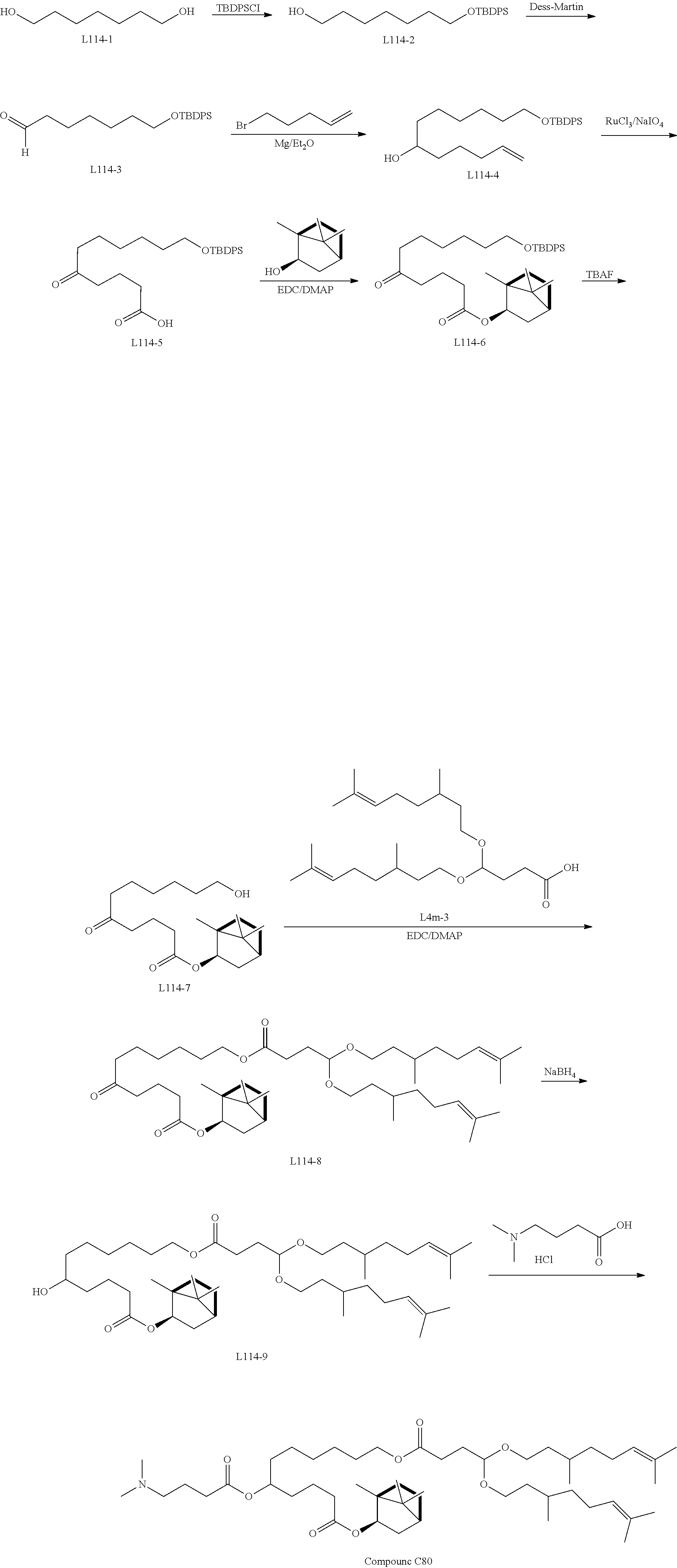

### Synthesis of 7-((tert-butyldiphenylsilyl)oxy)heptanal (L114-2)

Heptane-1,7-diol L114-1 (9.3 g, 70 mmol) was dissolved in ACN/hexane (60/180 mL), followed by addition of triethylamine (12 mL, 84 mmol) and TBDPSCl (18.3 mL, 70 mmol). The reaction mixture was then stirred at room temperature for 20 hours. The reaction mixture was then diluted by ethyl acetate (200 mL) and extracted by water (200 mL×2). The organic fraction was evaporated to obtain crude product which was then subjected to silica gel column using 0-20% EA in hexane as eluent to afford L114-2 as colorless liquid (16.3 g, 63%). ¹H-NMR (300 MHz, CDCl₃) δ 7.72-7.58 (m, 4H), 7.49-7.30 (m, 6H), 3.72-3.50 (m, 4H), 1.65-1.45 (m, 6H), 1.38-1.12 (m, 4H), 1.04 (s, 9H).

### Synthesis of 7-((tert-butyldiphenylsilyl)oxy)heptanal (L114-3)

7-((tert-butyldiphenylsilyl)oxy)heptan-1-ol L114-2 (16.3 g, 44 mmol) was dissolved in DCM (210 mL) and cooled to 0° C., followed by addition of Dess-Martin periodinane (20.5 g, 48 mmol). The reaction mixture was then stirred at room temperature for 2 h. When TLC showed completion of the reaction, Aq. NaOH (100 mL) was added to quench the reaction. The quenched reaction mixture was then diluted with DCM and washed with water twice. The organic fraction was evaporated to obtain crude product which was then subjected to silica gel column using 0-10% EA in hexane as eluent to afford L114-3 as colorless liquid (11.5 g, 71%). ¹H-NMR (300 MHz, CDCl₃) δ 9.75 (s, 1H), 7.72-7.58 (m, 4H), 7.49-7.30 (m, 6H), 3.64 (t, 2H), 2.47-2.30 (m, 2H), 1.65-1.45 (m, 6H), 1.38-1.12 (m, 4H), 1.04 (s, 9H).

### Synthesis of 12-((tert-butyldiphenylsilyl)oxy)dodec-1-en-6-ol (L114-4)

7-((tert-butyldiphenylsilyl)oxy)heptanal L114-3 (11.5 g, 31 mmol) was dissolved in THF (200 mL) and purged with N₂ for 15 min. Pent-4-en-1-ylmagnesium bromide (75 mL, 3.7 mmol, 0.5 M in THF) was then added dropwise to the aldehyde solution at room temperature. The resulting reaction mixture was stirred at room temperature for another hour. When TLC showed completion of the reaction, water (10 mL) was added. The solvent was evaporated, the mixture was dissolved in EA and extracted with sat. NH₄C₁. The organic fraction was evaporated to obtain crude product which was then subjected to silica gel column using 0-20% EA in hexane as eluent to afford L114-4 as colorless oil (13.5 g, 61%). ¹H-NMR (300 MHz, CDCl₃) δ 7.72-7.58 (m, 4H), 7.49-7.30 (m, 6H), 5.89-5.71 (m, 1H), 5.10-4.88 (m, 2H), 3.64 (t, 2H), 1.63-1.13 (m, 16H), 1.04 (s, 9H).

### Synthesis of 11-((tert-butyldiphenylsilyl)oxy)-5-oxoundecanoic acid (L114-5)

NaIO₄ (11.4 g, 8 eq) was dissolved in water (72 mL), followed by adding ACN (48 mL) and CCl₄ (48 mL). L114-4 (3 g, 6.8 mmol) was added to the above suspension and stirred for 5 min. RuCl₃ (138 mg, 0.68 mmol) was added in one portion. The resulting reaction mixture was stirred at room temperature for 20 h. The mixture was then diluted with water (100 mL) and extracted with DCM (100 mL×2). The organic fraction was collected and concentrated to dryness. The crude mixture was dissolved in acetone (20 mL). Jones reagent was then added slowly to the mixture until no color change can be observed. The reaction was then quenched with isopropanol (20 mL). The solvent was evaporated, and then the crude product was dissolved in DCM (50 mL) and extracted with water (50 mL×2). The organic fraction was evaporated to obtain crude product which was then subjected to silica gel column using 0-10% MeOH in DCM as eluent to afford L114-5 as colorless oil (1.7 g, 55%). %). ¹H-NMR (300 MHz, CDCl₃) δ 7.72-7.58 (m, 4H), 7.49-7.30 (m, 6H), 3.64 (t, 2H), 2.49 (t, 2H), 2.43-2.30 (m, 4H), 1.89 (p, 2H), 1.63-1.49 (m, 4H), 1.32-1.19 (m, 4H), 1.04 (s, 9H). CIMS m/z [M−H]⁺ 453.2.

### Synthesis of (1S,2R,4S)-1,7,7-trimethylbicyclo[2.2.1]heptan-2-yl 11-((tert-butyldiphenylsilyl) oxy)-5-oxoundecanoate (L114-6)

L114-5 (1.7 g, 3.7 mmol) was dissolved in DCM (60 mL), followed by addition of EDC/DMAP (2.87 g, 15 mmol/500 mg, 4.0 mmol). After stirring for 5 minutes, (S)-borneol (870 mg, 5.6 mmol) was added slowly to the above solution, and then the reaction mixture was stirred for 3 h at room temperature. When TLC showed completion of the reaction, the organic fraction was evaporated to obtain crude product which was subjected to silica gel column using 0-20% EA in hexane as eluent to afford L114-6 as colorless oil (1.6 g, 73%). ¹H-NMR (300 MHz, CDCl₃) δ 7.72-7.58 (m, 4H), 7.49-7.30 (m, 6H), 4.91-4.83 (m, 1H), 3.64 (t, 2H), 2.49 (t, 2H), 2.40-2.24 (m, 5H), 1.93-1.80 (m, 3H), 1.79-1.49 (m, 9H), 1.40-1.15 (m, 6H), 1.04 (s, 9H), 1.00-0.70 (m, 12H). CIMS m/z [M+H]⁺ 591.3.

### Synthesis (1S,2R,4S)-1,7,7-trimethylbicyclo[2.2.1]heptan-2-yl 11-hydroxy-5-oxoundecanoate (L114-7)

The starting material L114-6 (1.6 g, 2.7 mmol) was added to TBAF (1 M in THF, 5 mL). The resulting mixture was then stirred at room temperature for 2 h. When TLC showed the completion of reaction, the solvent was evaporated to obtain crude product which was subjected to silica gel column using 0-60% EA in hexane as eluent to afford L114-7 as light-yellow oil (830 mg, 87%). ¹H-NMR (300 MHz, CDCl₃) δ 4.91-4.83 (m, 1H), 3.64 (t, 2H), 2.49 (t, 2H), 2.40-2.24 (m, 5H), 1.93-1.80 (m, 3H), 1.79-1.49 (m, 8H), 1.40-1.15 (m, 8H), 1.00-0.70 (m, 10H).

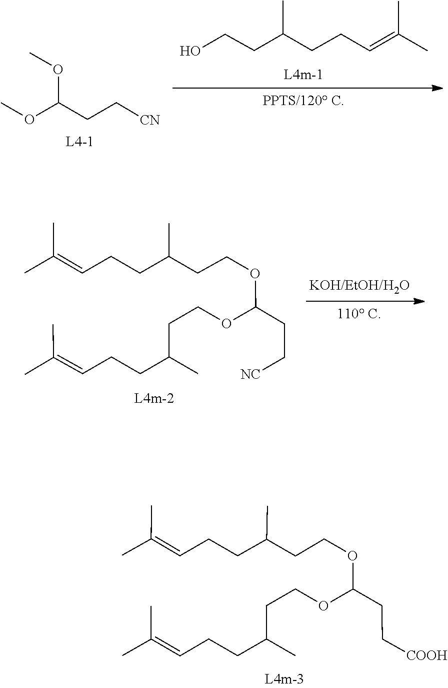

### Intermediate Step 1: Synthesis of 4,4-bis((3,7-dimethyloct-6-en-1-yl)oxy)butanenitrile (L4m-21

To a 100 mL round bottom flask, 4,4-dimethoxybutanenitrile L4-1 (1 equiv), alcohol L4m-1 (~2.5 equiv) and pyridinium p-toluenesulfonate (0.05 equiv) were added. The resulting mixture was stirred at 120° C. for 4 h and cooled to room temperature. EtOAc (50 mL) and H₂O (20 mL) were added in, and the resulting phases were separated. The aqueous phase was extracted with EtOAc (50 mL). Combined organic extracts were washed with H₂O (20 mL) and dried over anhydrous MgSO₄. Filtration and concentration provided crude material which was purified by flash column chromatography (SiO₂: 0 to 10% ethyl acetate in hexanes gradient) to yield L4m-2 as colorless oil (6.2 g, 73%). ¹H-NMR (300 MHz, CDCl₃) δ 5.08 (t, J=7.2 Hz, 2H), 4.54 (t, J=5.3 Hz, 1H), 3.72-3.36 (m, 4H), 2.41 (t, J=7.3 Hz, 2H), 2.10-1.84 (m, 6H), 1.80-1.05 (m, 22H), 0.89 (d, J=6.6 Hz, 6H).

### Intermediate Step 2: Synthesis of 4,4-bis((3,7-dimethyloct-6-en-1-yl)oxy)butanoic acid (L4m-31

To a 100 mL round bottom flask containing a solution of L4m-2 (1 equiv) in ethanol (50 mL) was added a solution of KOH in water (50 mL, 3 equiv KOH). The mixture was stirred at 120° C. for 20 h. The volatiles were removed, and the reaction pH was adjusted to 5. EtOAc (150 mL) and H₂O (60 mL) were added, and the resulting phases were separated. The aqueous phase was extracted with EtOAc (50 mL). Combined organic extracts were washed with H₂O (60 mL×2) and dried over anhydrous MgSO₄. Filtration and concentration provided L4m-3 as colorless oil (6.2 g, 73%). ¹H-NMR (300 MHz, CDCl₃) δ: 5.08 (t, J=6.84 Hz, 2H), 4.5 (t, J=5.22 Hz, 1H), 3.59 (m, 2H), 3.44 (m, 2H), 2.44 (t, J=7.44 Hz, 2H), 1.94 (m, 6H), 1.54-1.67 (m, 16H), 1.35 (m, 4H), 1.13 (m, 2H), 0.87 (d, J=6.33 Hz, 6H); CIMS m/z [M+H]⁺ 396.2.

### Synthesis of (1S,2R,4S)-1,7,7-trimethylbicyclo[2.2.1]heptan-2-yl 11-((4,4-bis((3,7-dimethyloct-6-en-1-yl)oxy)butanoyl)oxy)-5-oxoundecanoate (L114-8)

The starting acetal acid (1.11 g, 2.8 mmol) was dissolved in DCM (40 mL), followed by addition of EDC/DMAP (1.8 g, 9.2 mmol/313 mg, 2.5 mmol). After stirring 5 minutes, L114-7 (820 mg, 2.3 mmol) was added slowly to the above solution and the reaction mixture was stirred for 3 h at room temperature. When TLC showed completion of the reaction, the organic fraction was evaporated to obtain crude product which was subjected to silica gel column using 0-20% EA in hexane as eluent to afford L114-8 as colorless oil (1.6 g, 94%). ¹H-NMR (300 MHz, CDCl₃) δ 5.08 (t, 2H), 4.91-4.83 (m, 1H), 4.48 (t, 1H), 4.04 (t, 2H), 3.65-3.52 (m, 2H), 3.49-3.35 (m, 2H), 2.49 (t, 2H), 2.40-2.24 (m, 7H), 1.02-1.80 (m, 9H), 1.79-1.43 (m, 26H), 1.41-1.21 (m, 10H), 1.20-1.02 (m, 4H), 1.00-0.70 (m, 16H).

### Synthesis of (1S,2R,4S)-1,7,7-trimethylbicyclo[2.2.1]heptan-2-yl 11-((4,4-bis((3,7-dimethyloct-6-en-1-yl)oxy)butanoyl)oxy)-5-hydroxyundecanoate (L114-9)

L114-8 (1.6 g, 2.2 mmol) was added to THF/MeOH (24 mL/6 mL) and cooled to 0° C. NaBH₄ (95 mg, 2.4 mmol) was then added to the reaction. The resulting mixture was brought to room temperature and stirred for 3 h. When TLC showed completion of the reaction, the solvent was quenched by a few drops of H₂O, and evaporated to obtain crude product which was then subjected to silica gel column using 0-20% EA in hexane as eluent to afford L114-9 as light-yellow oil (1.45 g, 91%). ¹H-NMR (300 MHz, CDCl₃) δ 5.08 (t, 2H), 4.91-4.83 (m, 1H), 4.48 (t, 111), 4.04 (t, 2H), 3.65-3.52 (m, 3H), 3.49-3.35 (m, 2H), 2.49 (t, 2H), 2.40-2.24 (m, 5H), 1.02-1.80 (m, 8H), 1.83-1.07 (m, 48H), 1.00-0.70 (m, 18H).

### Synthesis of (1S,2R,4S)-1,7,7-trimethylbicyclo[2.2.1]heptan-2-yl 11-((4,4-bis((3,7-dimethyloct-6-en-1-yl)oxy)butanoyl)oxy)-5-hydroxyundecanoate (Compound C80)

To a 250 mL round bottom flask was added 4-(dimethylamino) butanoic acid hydrochloride (429 mg, 2.56 mmol), EDC (1.27 g, 6.63 mmol) and DMAP (116 mg, 0.95 mmol) in anhydrous dichloromethane (20 mL) and the reaction was stirred for 10 min. To this was added N, N-diisopropylethylamine (1.23 g, 1.65 mL, 9.48 mmol) along with L114-9 (695 mg, 0.95 mmol) in anhydrous dichloromethane (10 mL) and the reaction mixture was stirred under nitrogen at room temperature for 48 h. After completion of the reaction, about 20 g of flash silica (pre-neutralized with triethylamine) was added and the contents were stirred well to yield a uniform mixture. Solvent was removed from this mixture under vacuum. The residue was loaded on to an empty flash cartridge, which was then attached to a flash purification system loaded with 40 g flash silica column (equilibrated with 1% triethylamine in hexane) and was purified by flash chromatography (SiO₂: ethyl acetate/hexane (with 1% triethylamine) 0-20%) to get Compound C80 as a clear oil (634 mg, 79%). ¹H-NMR (300 MHz, CDCl₃) δ: 5.10 (t, J=7.0 Hz, 2H), 4.94-4.86 (m, 2H), 4.55 (t, J=7.0 Hz, 1H), 4.04 (t, J=7.0 Hz, 2H), 3.65-3.46 (m, 4H), 2.41-3.28 (m, 8H), 2.20 (s, 6H), 2.05-1.71 (m, 10H), 1.68-1.47 (m, 27H), 1.35-1.08 (m, 14H), 0.96-0.81 (m, 15H); CIMS m/z [M+H]⁺ 846.6. Analytical HPLC column: Agela Durashell C18, 4.6×50 mm, 3 m (Catalog No. DC930505-0), mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., ELSD, t_{R}=8.4 min, purity: >99%; UPLC column: Waters Aquity UPLC® CSHTM, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: mL/min, column temperature: 20±2° C., detector: CAD, t_{R}=12.13 min, purity: 94.13%.

### Example 2: LNP Formulations

Ionizable lipids, DSPC, cholesterol, and PEG-lipids were dissolved in pure ethanol in one of three formulation molar ratios (A, B, or C), with a total lipid concentration of 7.2 mM. A 0.067 mg polynucleotide/mL solution was prepared using acidic buffer (pH 4.0-5.0) containing Cas9 mRNA/sgRNA (1:1 ratio, SEQ ID NO: 35 and SEQ ID NO: 37 respectively, prepared as described in PCT Publication WO2020118041A1, which is incorporated by reference herein in its entirety). The nucleotide and lipid solutions were mixed at a 3:1 volume ratio using the NanoAssemblr microfluidic system at a 12 mL/min total flow rate resulting in rapid mixing and self-assembly of LNPs. Formulations were further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a Zetasizer Ultra (Malvern Panalytical). RNA encapsulation efficiency (EE %) was determined by Ribogreen assay.

### Formulation Molar Ratio A:

| Ionizable Lipid | DSPC | Cholesterol | DMG PEG-2k |
|---|---|---|---|
| 48.5 | 10 | 39 | 2.5 |

### Formulation Molar Ratio B:

| Ionizable Lipid | DSPC | Cholesterol | DMG PEG-2k |
|---|---|---|---|
| 55 | 10 | 32.5 | 2.5 |

### Formulation Molar Ratio C:

| Ionizable Lipid | DSPC | Cholesterol | DMPE PEG-2k |
|---|---|---|---|
| 55 | 10 | 32.5 | 2.5 |

Buffer X: 25 mM Sodium Acetate, pH 5.0; Buffer Y: 50 mM Citrate, pH 4.0

TABLE 2
LNP Formulations

| | Ionizable | Formulation | | | | |
|---|---|---|---|---|---|---|
| Form. | Lipid | Ratio | Buffer | Size (nm) | PDI | EE % |
| F-1 | 14 | B | Y | 78.8 | 0.043 | 87.2% |
| F-2 | CY61 | A | Y | 68.0 | 0.062 | 89.4% |
| F-3 | CY61 | B | Y | 79.9 | 0.002 | 88.7% |
| F-4 | 57 | C | X | 64.6 | 0.016 | 82.9% |
| F-5 | 52 | B | Y | 84.2 | 0.018 | 77.6% |
| F-6 | 55 | C | X | 66.5 | 0.032 | 83.6% |
| F-7 | 54 | C | X | 68.4 | 0.075 | 76.4% |
| F-8 | CY57 | A | Y | 89.3 | 0.143 | 77.5% |
| F-9 | 64 | B | Y | 85.8 | 0.034 | 84.9% |
| F-10 | 82 | A | Y | 69.7 | 0.066 | 89.4% |
| F-11 | 82 | B | X | 80.6 | 0.040 | 84.5% |
| F-12 | 75 | C | X | 62.7 | 0.018 | 86.6% |
| F-13 | 67 | A | Y | 71.9 | 0.041 | 83.6% |
| F-14 | 67 | B | Y | 82.7 | 0.030 | 85.7% |
| F-15 | 67 | C | X | 56.6 | 0.028 | 84.1% |
| F-16 | 66 | A | Y | 75.4 | 0.032 | 84.1% |
| F-19 | 66 | B | Y | 82.2 | 0.071 | 86.7% |
| F-20 | 66 | C | X | 62.0 | 0.047 | 86.9% |
| F-21 | 81 | B | Y | 85.5 | 0.011 | 74.9% |
| F-22 | CY69 | C | X | 60.2 | 0.074 | 73.4% |
| F-23 | 68 | A | Y | 70.6 | 0.006 | 78.5% |
| F-24 | 68 | B | Y | 83.9 | 0.008 | 82.5% |
| F-25 | 68 | C | X | 59.4 | 0.044 | 83.9% |
| F-26 | 69 | A | Y | 71.8 | 0.052 | 78.7% |
| F-27 | 69 | B | Y | 85.5 | 0.011 | 74.9% |
| F-28 | 77 | C | X | 61.5 | 0.057 | 77.5% |
| F-29 | 78 | A | Y | 75.7 | 0.113 | 89.2% |
| F-30 | 78 | B | Y | 76.4 | 0.024 | 80.8% |
| F-31 | 84 | A | Y | 103.1 | 0.131 | 86.2% |
| F-32 | CY65 | C | X | 57.0 | 0.072 | 79.9% |
| F-33 | 85 | B | Y | 82.0 | 0.019 | 90.7% |
| F-34 | C57 | A | Y | 91.0 | 0.060 | 78.5% |
| F-35 | C62 | A | Y | 90.2 | 0.044 | 85.8% |
| F-36 | C62 | B | Y | 116.6 | 0.078 | 77.5% |
| F-37 | C65 | C | X | 76.0 | 0.030 | 84.1% |
| F-38 | C56 | A | Y | 89.9 | 0.053 | 86.3% |
| F-39 | C56 | B | Y | 115.4 | 0.065 | 86.1% |
| F-40 | C59 | A | Y | 107.3 | 0.106 | 85.7% |
| F-41 | C59 | B | Y | 130.7 | 0.021 | 77.0% |
| F-42 | C66 | A | Y | 87.4 | 0.081 | 86.6% |
| F-43 | C66 | B | Y | 101.9 | 0.008 | 86.4% |
| F-44 | C66 | C | Y | 86.4 | 0.093 | 82.8% |

### Example 3: In Vivo Editing Delivery in Mice—TTR KD

### In Vivo Protocol

CD-I female mice, ranging from 6-10 weeks of age were used in each study. LNPs were dosed via the lateral tail vein in a volume of approximately 5 mL per kilogram body weight. The animals were periodically observed for adverse effects for at least 24 hours post dose. Mice were dosed at 0.2 mpk. Each formulation was dosed in 5 animals. Animals were euthanized at 7 days by exsanguination via cardiac puncture under isoflurane anesthesia. Liver tissue was collected from each animal for DNA extraction and analysis. Blood was collected into serum separator tubes or into tubes containing buffered sodium citrate for plasma as described herein. Cohorts of mice were measured for editing by Next-Generation Sequencing (NGS).

### Transthyretin (TTR) ELISA Analysis

Blood was collected and the serum was isolated as indicated. The total mouse TTR serum levels were determined using a Mouse Prealbumin (Transthyretin) ELISA Kit (Aviva Systems Biology, Cat. OKIAOOl 11). Briefly, sera were serial diluted with kit sample diluent, e.g. to a final dilution of 10,000-fold and/or 2,500-fold. The diluted sample was then added to the ELISA plates and the assay was then carried out according to directions. Serum TTR data from treatment groups are expressed as a percentage of day 0 TTR levels. Lower percentage values correlate with greater editing efficiency.

### Key: *<5%; 5%≤**<20%; 20%≤***<50%; 50%≤****<75%; 75%≤*****<100%

TABLE 3
Gene Editing Data - Mouse

| | Ionizable | |
|---|---|---|
| Form. | Lipid | TTR (% of pre-dose levels) |
| F-1 | 14 | *** |
| F-2 | CY61 | *** |
| F-3 | CY61 | **** |
| F-4 | 57 | **** |
| F-5 | 52 | * |
| F-6 | 55 | ***** |
| F-7 | 54 | ***** |
| F-8 | CY57 | *** |
| F-9 | 64 | ** |
| F-10 | 82 | ** |
| F-11 | 82 | *** |
| F-12 | 75 | **** |
| F-13 | 67 | * |
| F-14 | 67 | ** |
| F-15 | 67 | **** |
| F-16 | 66 | * |
| F-19 | 66 | *** |
| F-20 | 66 | **** |
| F-21 | 81 | *** |
| F-22 | CY69 | **** |
| F-23 | 68 | * |
| F-24 | 68 | * |
| F-25 | 68 | *** |
| F-26 | 69 | ** |
| F-27 | 69 | ** |
| F-28 | 77 | **** |
| F-29 | 78 | * |
| F-30 | 78 | * |
| F-31 | 84 | *** |
| F-32 | CY65 | **** |
| F-33 | 85 | ***** |
| F-34 | C57 | ** |
| F-35 | C62 | * |
| F-36 | C62 | *** |
| F-37 | C65 | **** |
| F-38 | C56 | * |
| F-39 | C56 | *** |
| F-40 | C59 | ** |
| F-41 | C59 | *** |
| F-42 | C66 | ** |
| F-43 | C66 | ** |
| F-44 | C66 | ** |

### NGS Sequencing

In brief, to quantitatively determine the efficiency of editing at the target location in the genome, genomic DNA was isolated and deep sequencing was utilized to identify the presence of insertions and deletions (“indels”) introduced by gene editing.

PCR primers were designed around the target site (e.g., B2M), and the genomic area of interest was amplified. Additional PCR was performed according to the manufacturer's protocols (Illumina) to add the necessary chemistry for sequencing. The amplicons were sequenced on an Illumina NextSeq 2000 instrument. The reads were aligned to the relevant reference genome (e.g., GRCm38) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing”) is defined as the total number of sequence reads with insertions or deletions over the total number of sequence reads, including wild type.

Editing percentage data (Indel %) collected from the NGS sequencing for select formulations amongst Formulations F-1 through F-44 strongly correlated with the TTR ELISA assay data reported in Table 3.

### Example 4: In Vivo Editing Delivery in Non-Human Primates—TTR KD

### In Vivo Protocol

LNP formulations were prepared as described in Example 2. Each formulation was administered to 3 non-naïve Cynomolgus monkeys on Day 1 via 60-min intravenous infusion into an appropriate peripheral vein using an infusion pump at a dose level of 2.0 mg/kg (dose volume of 5 mL/kg; concentration 0.4 mg/mL). Samples were collected throughout the study for clinical pathology parameters, pharmacokinetic/pharmacodynamic (PK/PD) analysis and immunostimulation. Blood samples were collected daily up to study day 29 and on day 29, the test subjects were terminated. Test subject livers and spleens were collected for NGS analysis.

### NGS Sequencing

NGS sequencing was carried out as described in Example 3.

### Transthyretin (TTR) Analysis

The TTR concentration in cynomolgus monkey plasma samples was measured using an LC/MS/MS method. A Sciex Triple Quad 7500 mass spectrometer (Framingham, MA) coupled with a Waters Acquity UPLC (Milford, MA) was used for samples analysis. A BEH C18 columns (Waters, Milford, MA) was used for chromatographic separation. A recombinant monkey TTR protein was used as reference material (ab239566, abcam, Waltham, MA). Plasma samples were digested by trypsin. A selected tryptic peptide was used to quantitate TTR. Stable isotopic labelled internal standards were used as internal standard.

### Data Summary

An LNP formulation selected from Formulations F-1 through F-33 containing 1:1 Cas9:gRNA(TTR), utilizing formulation ratio A, was dosed in 3 non-naïve male cynomolgus monkeys at 2.0 mg/kg bodyweight via single dose IV infusion over 60 minutes, as discussed above. Blood samples were collected on days 4, 7, 14, 21 and 29 and on day 29, the test subjects were terminated and their livers and spleens were collected. TTR ELISA analysis of serum samples collected at days 4, 7, 14, and 29 showed average TTR KD of 42%, 22%, 13%, and 16%, respectively, demonstrating effective knockdown of TTR expression by the test formulation. NGS sequencing analysis on day 29 of liver and spleen samples showed 63.1% and 8.7% indel editing efficiency, respectively.

SEQ ID LISTING

| SEQ ID | | |
|---|---|---|
| NO | Description | Sequence |
| SEQ ID | Cas9 | GGGUCCCGCAGUCGGCGUCCAGCGGCUCUGCUUGUUCGUG |
| NO: 35 | mRNA; | UGUGUGUCGUUGCAGGCCUUAUUCGGAUCCGCCACCAUGG |
| | All “U” = | ACAAGAAGUACAGCAUCGGACUGGACAUCGGAACAAACAG |
| | N-1- | CGUCGGAUGGGCAGUCAUCACAGACGAAUACAAGGUCCCG |
| | Methyl- | AGCAAGAAGUUCAAGGUCCUGGGAAACACAGACAGACACA |
| | psuedouridine | GCAUCAAGAAGAACCUGAUCGGAGCACUGCUGUUCGACAG |
| | | CGGAGAAACAGCAGAAGCAACAAGACUGAAGAGAACAGCA |
| | | AGAAGAAGAUACACAAGAAGAAAGAACAGAAUCUGCUACC |
| | | UGCAGGAAAUCUUCAGCAACGAAAUGGCAAAGGUCGACGA |
| | | CAGCUUCUUCCACAGACUGGAAGAAAGCUUCCUGGUCGAA |
| | | GAAGACAAGAAGCACGAAAGACACCCGAUCUUCGGAAACA |
| | | UCGUCGACGAAGUCGCAUACCACGAAAAGUACCCGACAAU |
| | | CUACCACCUGAGAAAGAAGCUGGUCGACAGCACAGACAAG |
| | | GCAGACCUGAGACUGAUCUACCUGGCACUGGCACACAUGA |
| | | UCAAGUUCAGAGGACACUUCCUGAUCGAAGGAGACCUGAA |
| | | CCCGGACAACAGCGACGUCGACAAGCUGUUCAUCCAGCUG |
| | | GUCCAGACAUACAACCAGCUGUUCGAAGAAAACCCGAUCA |
| | | ACGCAAGCGGAGUCGACGCAAAGGCAAUCCUGAGCGCAAG |
| | | ACUGAGCAAGAGCAGAAGACUGGAAAACCUGAUCGCACAG |
| | | CUGCCGGGAGAAAAGAAGAACGGACUGUUCGGAAACCUGA |
| | | UCGCACUGAGCCUGGGACUGACACCGAACUUCAAGAGCAA |
| | | CUUCGACCUGGCAGAAGACGCAAAGCUGCAGCUGAGCAAG |
| | | GACACAUACGACGACGACCUGGACAACCUGCUGGCACAGA |
| | | UCGGAGACCAGUACGCAGACCUGUUCCUGGCAGCAAAGAA |
| | | CCUGAGCGACGCAAUCCUGCUGAGCGACAUCCUGAGAGUC |
| | | AACACAGAAAUCACAAAGGCACCGCUGAGCGCAAGCAUGA |
| | | UCAAGAGAUACGACGAACACCACCAGGACCUGACACUGCU |
| | | GAAGGCACUGGUCAGACAGCAGCUGCCGGAAAAGUACAAG |
| | | GAAAUCUUCUUCGACCAGAGCAAGAACGGAUACGCAGGAU |
| | | ACAUCGACGGAGGAGCAAGCCAGGAAGAAUUCUACAAGUU |
| | | CAUCAAGCCGAUCCUGGAAAAGAUGGACGGAACAGAAGAA |
| | | CUGCUGGUCAAGCUGAACAGAGAAGACCUGCUGAGAAAGC |
| | | AGAGAACAUUCGACAACGGAAGCAUCCCGCACCAGAUCCA |
| | | CCUGGGAGAACUGCACGCAAUCCUGAGAAGACAGGAAGAC |
| | | UUCUACCCGUUCCUGAAGGACAACAGAGAAAAGAUCGAAA |
| | | AGAUCCUGACAUUCAGAAUCCCGUACUACGUCGGACCGCU |
| | | GGCAAGAGGAAACAGCAGAUUCGCAUGGAUGACAAGAAAG |
| | | AGCGAAGAAACAAUCACACCGUGGAACUUCGAAGAAGUCG |
| | | UCGACAAGGGAGCAAGCGCACAGAGCUUCAUCGAAAGAAU |
| | | GACAAACUUCGACAAGAACCUGCCGAACGAAAAGGUCCUG |
| | | CCGAAGCACAGCCUGCUGUACGAAUACUUCACAGUCUACA |
| | | ACGAACUGACAAAGGUCAAGUACGUCACAGAAGGAAUGAG |
| | | AAAGCCGGCAUUCCUGAGCGGAGAACAGAAGAAGGCAAUC |
| | | GUCGACCUGCUGUUCAAGACAAACAGAAAGGUCACAGUCA |
| | | AGCAGCUGAAGGAAGACUACUUCAAGAAGAUCGAAUGCUU |
| | | CGACAGCGUCGAAAUCAGCGGAGUCGAAGACAGAUUCAAC |
| | | GCAAGCCUGGGAACAUACCACGACCUGCUGAAGAUCAUCA |
| | | AGGACAAGGACUUCCUGGACAACGAAGAAAACGAAGACAU |
| | | CCUGGAAGACAUCGUCCUGACACUGACACUGUUCGAAGAC |
| | | AGAGAAAUGAUCGAAGAAAGACUGAAGACAUACGCACACC |
| | | UGUUCGACGACAAGGUCAUGAAGCAGCUGAAGAGAAGAAG |
| | | AUACACAGGAUGGGGAAGACUGAGCAGAAAGCUGAUCAAC |
| | | GGAAUCAGAGACAAGCAGAGCGGAAAGACAAUCCUGGACU |
| | | UCCUGAAGAGCGACGGAUUCGCAAACAGAAACUUCAUGCA |
| | | GCUGAUCCACGACGACAGCCUGACAUUCAAGGAAGACAUC |
| | | CAGAAGGCACAGGUCAGCGGACAGGGAGACAGCCUGCACG |
| | | AACACAUCGCAAACCUGGCAGGAAGCCCGGCAAUCAAGAA |
| | | GGGAAUCCUGCAGACAGUCAAGGUCGUCGACGAACUGGUC |
| | | AAGGUCAUGGGAAGACACAAGCCGGAAAACAUCGUCAUCG |
| | | AAAUGGCAAGAGAAAACCAGACAACACAGAAGGGACAGAA |
| | | GAACAGCAGAGAAAGAAUGAAGAGAAUCGAAGAAGGAAUC |
| | | AAGGAACUGGGAAGCCAGAUCCUGAAGGAACACCCGGUCG |
| | | AAAACACACAGCUGCAGAACGAAAAGCUGUACCUGUACUA |
| | | CCUGCAGAACGGAAGAGACAUGUACGUCGACCAGGAACUG |
| | | GACAUCAACAGACUGAGCGACUACGACGUCGACCACAUCG |
| | | UCCCGCAGAGCUUCCUGAAGGACGACAGCAUCGACAACAA |
| | | GGUCCUGACAAGAAGCGACAAGAACAGAGGAAAGAGCGAC |
| | | AACGUCCCGAGCGAAGAAGUCGUCAAGAAGAUGAAGAACU |
| | | ACUGGAGACAGCUGCUGAACGCAAAGCUGAUCACACAGAG |
| | | AAAGUUCGACAACCUGACAAAGGCAGAGAGAGGAGGACUG |
| | | AGCGAACUGGACAAGGCAGGAUUCAUCAAGAGACAGCUGG |
| | | UCGAAACAAGACAGAUCACAAAGCACGUCGCACAGAUCCU |
| | | GGACAGCAGAAUGAACACAAAGUACGACGAAAACGACAAG |
| | | CUGAUCAGAGAAGUCAAGGUCAUCACACUGAAGAGCAAGC |
| | | UGGUCAGCGACUUCAGAAAGGACUUCCAGUUCUACAAGGU |
| | | CAGAGAAAUCAACAACUACCACCACGCACACGACGCAUACC |
| | | UGAACGCAGUCGUCGGAACAGCACUGAUCAAGAAGUACCC |
| | | GAAGCUGGAAAGCGAAUUCGUCUACGGAGACUACAAGGUC |
| | | UACGACGUCAGAAAGAUGAUCGCAAAGAGCGAACAGGAAA |
| | | UCGGAAAGGCAACAGCAAAGUACUUCUUCUACAGCAACAU |
| | | CAUGAACUUCUUCAAGACAGAAAUCACACUGGCAAACGGA |
| | | GAAAUCAGAAAGAGACCGCUGAUCGAAACAAACGGAGAAA |
| | | CAGGAGAAAUCGUCUGGGACAAGGGAAGAGACUUCGCAAC |
| | | AGUCAGAAAGGUCCUGAGCAUGCCGCAGGUCAACAUCGUC |
| | | AAGAAGACAGAAGUCCAGACAGGAGGAUUCAGCAAGGAAA |
| | | GCAUCCUGCCGAAGAGAAACAGCGACAAGCUGAUCGCAAG |
| | | AAAGAAGGACUGGGACCCGAAGAAGUACGGAGGAUUCGAC |
| | | AGCCCGACAGUCGCAUACAGCGUCCUGGUCGUCGCAAAGG |
| | | UCGAAAAGGGAAAGAGCAAGAAGCUGAAGAGCGUCAAGGA |
| | | ACUGCUGGGAAUCACAAUCAUGGAAAGAAGCAGCUUCGAA |
| | | AAGAACCCGAUCGACUUCCUGGAAGCAAAGGGAUACAAGG |
| | | AAGUCAAGAAGGACCUGAUCAUCAAGCUGCCGAAGUACAG |
| | | CCUGUUCGAACUGGAAAACGGAAGAAAGAGAAUGCUGGCA |
| | | AGCGCAGGAGAACUGCAGAAGGGAAACGAACUGGCACUGC |
| | | CGAGCAAGUACGUCAACUUCCUGUACCUGGCAAGCCACUA |
| | | CGAAAAGCUGAAGGGAAGCCCGGAAGACAACGAACAGAAG |
| | | CAGCUGUUCGUCGAACAGCACAAGCACUACCUGGACGAAA |
| | | UCAUCGAACAGAUCAGCGAAUUCAGCAAGAGAGUCAUCCU |
| | | GGCAGACGCAAACCUGGACAAGGUCCUGAGCGCAUACAAC |
| | | AAGCACAGAGACAAGCCGAUCAGAGAACAGGCAGAAAACA |
| | | UCAUCCACCUGUUCACACUGACAAACCUGGGAGCACCGGCA |
| | | GCAUUCAAGUACUUCGACACAACAAUCGACAGAAAGAGAU |
| | | ACACAAGCACAAAGGAAGUCCUGGACGCAACACUGAUCCA |
| | | CCAGAGCAUCACAGGACUGUACGAAACAAGAAUCGACCUG |
| | | AGCCAGCUGGGAGGAGACGGAGGAGGAAGCCCGAAGAAGA |
| | | AGAGAAAGGUCUAGCUAGCCAUCACAUUUAAAAGCAUCUC |
| | | AGCCUACCAUGAGAAUAAGAGAAAGAAAAUGAAGAUCAAU |
| | | AGCUUAUUCAUCUCUUUUUCUUUUUCGUUGGUGUAAAGCC |
| | | AACACCCUGUCUAAAAAACAUAAAUUUCUUUAAUCAUUUU |
| | | GCCUCUUUUCUCUGUGCUUCAAUUAAUAAAAAAUGGAAAG |
| | | AACCUCGAGAAAAAAAAAAAAAAAAAAAAAAAAAAAAAAA |
| | | AAAAAAAAAAAAAAAAAAAAAAAAAAAAAAAAAAAAAAA |
| | | AAAAAAAAAAAAAAAAAAAAUCUAG |
| SEQ ID | Cas9 AA | MDKKYSIGLDIGTNSVGWAVITDEYKVPSKKFKVLGNTDRHSI |
| NO: 36 | | KKNLIGALLFDSGETAEATRLKRTARRRYTRRKNRICYLQEIFS |
| | | NEMAKVDDSFFHRLEESFLVEEDKKHIERHPIFGNIVDEVAYIIEK |
| | | YPTIYHLRKKLVDSTDKADLRLIYLALAHMIKFRGHFLIEGDLN |
| | | PDNSDVDKLFIQLVQTYNQLFEENPINASGVDAKAILSARLSKS |
| | | RRLENLIAQLPGEKKNGLFGNLIALSLGLTPNFKSNFDLAEDAK |
| | | LQLSKDTYDDDLDNLLAQIGDQYADLFLAAKNLSDAILLSDILR |
| | | VNTEITKAPLSASMIKRYDEHHQDLTLLKALVRQQLPEKYKEIF |
| | | FDQSKNGYAGYIDGGASQEEFYKFIKPILEKMDGTEELLVKLNR |
| | | EDLLRKQRTFDNGSIPHQIHLGELHAILRRQEDFYPFLKDNREKI |
| | | EKILTFRIPYYVGPLARGNSRFAWMTRKSEETITPWNFEEVVDK |
| | | GASAQSFIERMTNFDKNLPNEKVLPKHSLLYEYFTVYNELTKV |
| | | KYVTEGMRKPAFLSGEQKKAIVDLLFKTNRKVTVKQLKEDYFK |
| | | KIECFDSVEISGVEDRFNASLGTYHDLLKIIKDKDFLDNEENEDI |
| | | LEDIVLTLTLFEDREMIEERLKTYAHLFDDKVMKQLKRRRYTG |
| | | WGRLSRKLINGIRDKQSGKTILDFLKSDGFANRNFMQLIHDDSL |
| | | TFKEDIQKAQVSGQGDSLHEHIANLAGSPAIKKGILQTVKVVDE |
| | | LVKVMGRHKPENIVIEMARENQTTQKGQKNSRERMKRIEEGIK |
| | | ELGSQILKEHPVENTQLQNEKLYLYYLQNGRDMYVDQELDINR |
| | | LSDYDVDHIVPQSFLKDDSIDNKVLTRSDKNRGKSDNVPSEEVV |
| | | KKMKNYWRQLLNAKLITQRKFDNLTKAERGGLSELDKAGFIK |
| | | RQLVETRQITKHVAQILDSRMNTKYDENDKLIREVKVITLKSKL |
| | | VSDFRKDFQFYKVREINNYHHAHDAYLNAVVGTALIKKYPKLE |
| | | SEFVYGDYKVYDVRKMIAKSEQEIGKATAKYFFYSNIMNFFKT |
| | | EITLANGEIRKRPLIETNGETGEIVWDKGRDFATVRKVLSMPQV |
| | | NIVKKTEVQTGGFSKESILPKRNSDKLIARKKDWDPKKYGGFDS |
| | | PTVAYSVLVVAKVEKGKSKKLKSVKELLGITIMERSSFEKNPID |
| | | FLEAKGYKEVKKDLIIKLPKYSLFELENGRKRMLASAGELQKG |
| | | NELALPSKYVNFLYLASHYEKLKGSPEDNEQKQLFVEQHKHYL |
| | | DEIIEQISEFSKRVILADANLDKVLSAYNKHRDKPIREQAENIIHL |
| | | FTLTNLGAPAAFKYFDTTIDRKRYTSTKEVLDATLIHQSITGLYE |
| | | TRIDLSQLGGDGGGSPKKKRKV |
| SEQ ID | sgRNA | 5′-ascsasCAAAUACCAGUCCAGCGGUUUUAGAgcuagaaauag |
| NO: 37 | A, G, U, C: | cAAGUUAAAAUAAGGCUAGUCCGUUAUCaacuugaaaaagug |
| | unmodified | gcaccgagucggugcusususu-3′ |
| | RNA | |
| | nucleotide | |
| | a, g, u, c: | |
| | 2′-O- | |
| | Methyl- | |
| | nucleotide | |
| | s: | |
| | Phosphoro | |
| | thioate | |
| | linkage | |

1. A pharmaceutical composition comprising:
a) at least one lipid nanoparticle comprising:
i) at least one ionizable lipid selected from a compound of Formula (CY-VI″);
Formula (X) or Formula (IC); and
b) at least one nucleobase editing system.

2. The pharmaceutical composition of claim 1, wherein the nucleobase editing system comprises a CRISPR-Cas gene editing system.

3. The pharmaceutical composition of claim 2, wherein the nucleobase editing system comprises a Type V CRISPR-Cas gene editing system.

4. The pharmaceutical composition of claim 1, wherein the nucleobase editing system comprises a prime editing system or components thereof.

5. The pharmaceutical composition of claim 1, wherein the nucleobase editing system comprises a retron editing system.

6. The pharmaceutical composition of claim 1, wherein the nucleobase editing system comprises a TnpB editing system.

7. The pharmaceutical composition of claim 1, wherein the nucleobase editing system comprises an integrase editing system.

8. The pharmaceutical composition of claim 1, wherein the nucleobase editing system comprises a base editing system.

9. The pharmaceutical composition of claim 1, wherein the nucleobase editing system comprises an epigenetic editing system.

10. The pharmaceutical composition of claim 1, wherein the nucleobase editing system comprises a gene writing system.

11. The pharmaceutical composition of claim 1, wherein the nucleobase editing system comprises a gene inactivating system.

12. The pharmaceutical composition of claim 1, wherein the nucleobase editing system comprises zinc finger nuclease.

13. The pharmaceutical composition of claim 1, wherein the nucleobase editing system comprises a TALE Nuclease, a TALE nickase, Zinc Finger (ZF) Nuclease, ZF Nickase, meganuclease, or a combination thereof.

14. The pharmaceutical composition of claim 1, wherein the nucleobase editing system comprises a meganuclease.

15. The pharmaceutical composition of any one of claims 1-14, wherein the at least one lipid nanoparticle further comprises:
i) at least one structural lipid;
ii) at least one phospholipid; and
iii) at least one PEGylated lipid.

16. The pharmaceutical composition of any of one of claims 1-15, wherein the at least one structural lipid is selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and any combinations thereof.

17. The pharmaceutical composition of any one of claims 1-16, wherein the at least one phospholipid is selected from 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium (S)-2-ammonio-3-((((R)-2-(oleoyloxy)-3-(stearoyloxy)propoxy)oxidophosphoryl)oxy)propanoate (L-a-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin.

18. The pharmaceutical composition of any one of claims 1-17, wherein the at least one PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)₂₀₀₀)propylcarbamate, (PEG)-C-DOMG, PEG-C-DMA, and DSPE-PEG-X.

19. The pharmaceutical composition of any one of claims 1-18, wherein the LNP further comprises at least one additional lipid component selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Nα-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-α-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide](MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g.1,2-dioleoyl-sn-glycero-3-phosphoethanolamine-N-[4-(p-maleimidophenyl)but-yramid (MPB-PE), Nervonic Acid (NA), sodium cholate (NaChol), 1,2-dioleoyl-sn-glycero-3-[phosphoethanolamine-N-dodecanoyl (NC12-DOPE), I-oleoyl-2-cholesteryl hemisuccinoyl-sn-glycero-3-phosphocholine (OChemsPC), phosphatidylethanolamine lipid (PE), PE lipid conjugated with polyethylene glycol(PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.

20. A method of delivering a nucleobase editing system to a subject in need thereof, the method comprising administering to the subject the pharmaceutical composition of any one of claims 1-19.

21. The pharmaceutical composition of any of claims 1-19 for use as a medicament.

22. Use of a pharmaceutical composition of claims 1-19 for the manufacture of a medicament for delivery of a nucleobase editing system.


SEQUENCE LISTING (37 sequences)

SEQ ID NO: 1 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 2 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 3 - RNA, 83 nt, synthetic construct
  source 1..83: mol_type = other RNA
ggaaatcgcaaaatttttgctctttttcgcgttagatttcttttagttttctykcaactagcaagctttttgttctcgccrcc

SEQ ID NO: 4 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 5 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 6 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 7 - RNA, 59 nt, synthetic construct
  source 1..59: mol_type = other RNA
ggagaacttccgcttccgttggcgcaagcgctttcattttttctgctaccgtgactaag

SEQ ID NO: 8 - RNA, 46 nt, synthetic construct
  source 1..46: mol_type = other RNA
ggaaataagagagaaaagaagagtaagaagaaatataagagccacc

SEQ ID NO: 9 - RNA, 56 nt, synthetic construct
  source 1..56: mol_type = other RNA
ggaaataagagagaaaagaagagtaagaagaaatataagaccccggcgccgccacc

SEQ ID NO: 10 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 11 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 12 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 13 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 14 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 15 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 16 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 17 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 18 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 19 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 20 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 21 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 22 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 23 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 24 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 25 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 26 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 27 - RNA, 57 nt, synthetic construct
  source 1..57: mol_type = other RNA
ggaaataagagagaaaagaagagtaagaagaaatataagagccaaaaaaaaaaaacc

SEQ ID NO: 28 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 29 - RNA, 11 nt, synthetic construct
  source 1..11: mol_type = other RNA
gggatcctacc

SEQ ID NO: 30 - RNA, 47 nt, synthetic construct
  source 1..47: mol_type = other RNA
gggaaataagagagaaaagaagagtaagaagaaatataagagccacc

SEQ ID NO: 31 - RNA, 15 nt, synthetic construct
  source 1..15: mol_type = other RNA
taagcccctccgggg

SEQ ID NO: 32 - RNA, 15 nt, synthetic construct
  source 1..15: mol_type = other RNA
taaagctccccgggg

SEQ ID NO: 33 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 34 - protein, 2080 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAI

SEQ ID NO: 35 - RNA, 4506 nt, synthetic construct
  source 1..4506: mol_type = other RNA
gggtcccgcagtcggcgtccagcggctctgcttgttcgtgtgtgtgtcgttgcaggccttattcggatccgccaccatggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 36 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 37 - RNA, 100 nt, synthetic construct
  source 1..100: mol_type = other RNA
acacaaataccagtccagcggttttagagctagaaatagcaagttaaaataaggctagtccgttatcaacttgaaaaagtggcaccgagtcggtgctttt